(19) United States
(12) Patent Application Publication
(10) Pub. No.: US 2025/0129103 A1
(43) Pub. Date: Apr. 24, 2025

(54) METHODS FOR TREATMENT OF CANCER

(71) Applicant: Frontier Medicines Corporation, South San Francisco, CA (US)

(72) Inventors: Snahel Patel, Foster City, CA (US); Philip A. Gerken, San Francisco, CA (US); Monika Jane Williams, Woodside, CA (US)

(21) Appl. No.: 18/836,614

(22) PCT Filed: Feb. 7, 2023
(86) PCT No.: PCT/US2023/012544

§ 371 Date: Aug. 7, 2024

Related U.S. Application Data
(60) Provisional application No. 63/267,663, filed on Feb. 7, 2022.

(51) Int. Cl. C07D 519/00; A61K 31/517; A61K 31/519; A61K 31/5377; A61K 31/5386; A61K 31/541; A61K 31/553; A61K 45/06; C07D 487/04

(52) U.S. Cl. CPC C07D 519/00; A61K 31/517; A61K 31/519; A61K 31/5377; A61K 31/5386; A61K 31/541; A61K 31/553; A61K 45/06; C07D 487/04

(57) ABSTRACT

The present disclosure provides compounds and methods useful in the treatment and suppression of cancer, for example, useful for treating or suppressing cancers characterized by KRAS G12C. Also provided are pharmaceutical compositions containing such compounds and processes for preparing such compounds.

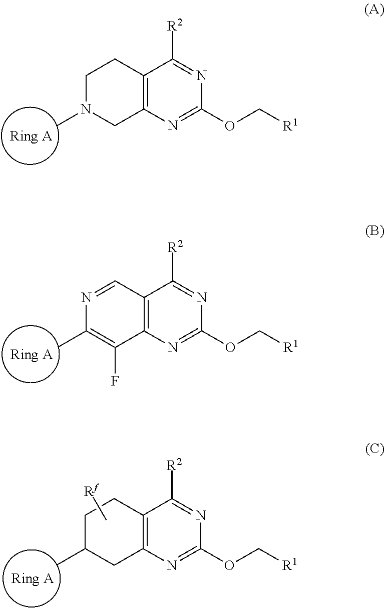

### CROSS-REFERENCE TO RELATED APPLICATION

This application claims priority to U.S. Provisional Application No. 63/267,663, filed on Feb. 7, 2022, which is incorporated by reference herein in its entirety and for all purposes.

### FIELD OF THE DISCLOSURE

The present disclosure provides compounds useful in treating or suppressing cancer, and in particular, useful in treating or suppressing cancers characterized by the KRAS G12C mutant.

Also provided are pharmaceutical formulations containing such compounds, processes for preparing such compounds, and methods of using such compounds in the treatment or suppression of cancers.

### BACKGROUND

KRAS is a molecular switch. Under normal physiological conditions, the protein is bound to guanosine diphosphate (GDP) in the “off state.” In response to signaling through receptor tyrosine kinases (RTKs) such as EGFR, the GDP is exchanged to guanosine triphosphate (GTP) in a process facilitated by guanine nucleotide exchange factors (GEFs) such as SOS. The GTP-bound form of KRAS is in the “on state,” and interacts with proteins such as RAF and PI3K to promote downstream signaling that leads to cell proliferation and survival. KRAS can slowly hydrolyze GTP back to GDP, thus returning to the off-state, in a process facilitated by GAPs (GTPase-activating Proteins).

KRAS mutations are found in approximately 30% of all human cancers, and are highly prevalent among three of the deadliest forms of cancer: pancreatic (95%), colorectal (45%), and lung (35%). Together, these cancers occur in more than 200,000 patients annually in the US alone. One particular mutation, a glycine to cysteine substitution at position 12 (G12C), occurs in more than 40,000 patients per year. The KRAS G12C mutation impairs hydrolysis of GTP to GDP, thus trapping KRAS in the on-state and promoting cancer cell proliferation.

The cysteine residue of G12C provides an opportunity to develop targeted covalent drugs for this mutant KRAS. Early clinical trial results for KRAS G12C inhibitors AMG 510 and MRTX849 have shown encouraging results for non-small cell lung cancer (NSCLC), but the data are less compelling for colorectal cancer (CRC). Moreover, even in cases where patients respond to initial treatment, there are signs that the response may be limited in duration and that resistance could arise rapidly.

Most inhibitors of KRAS mutants bind preferentially to the GDP-bound form of the protein. For example, Amgen KRAS inhibitor AMG 510 and Mirati KRAS inhibitor MRTX849 react with the GDP-bound form of KRAS G12C at least 1000-fold more rapidly than with the GTP-bound form of the protein. One form of resistance that has been observed is for cancer cells to increase signaling through RTKs, thus increasing the amount of GTP-bound KRAS, which is less affected by current inhibitors. Thus, creating a molecule that could bind to and inhibit both the GDP- and GTP-bound forms of KRAS could have substantial utility.

What is needed are compounds useful in the treatment of cancer, such as cancers characterized by KRAS G12C. What is further needed are compounds useful in the treatment of cancers characterized by KRAS G12C, wherein the compounds bind to and inhibit both the inactive GDP- and activated GTP-bound forms of KRAS. What is further needed are compounds useful in the treatment of cancers characterized by KRAS G12C, wherein the compound has improved inhibition of the GTP-bound form of KRAS G12C.

### SUMMARY

In one aspect, the invention provides a compound of Formula (A), Formula (B) or Formula (C):

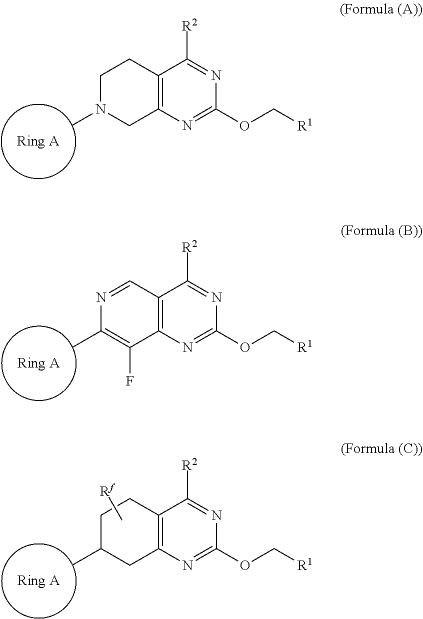

or a salt thereof, and/or an isotopologue thereof, wherein:
Ring A is a 6-10 membered aryl or a 5-10 membered heteroaryl, each substituted with 0, 1, 2 or 3 substituents independently selected from halo, —OH, —NH₂, C₁-C₄ alkyl, C₁-C₄ alkenyl, C₁-C₄ alkoxy, C₁-C₄ haloalkyl, C₁-C₄ haloalkoxy and C₂-C₃ alkynyl;
R¹ is

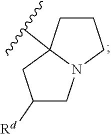

R^{d} is H, —F or —OH;
R² is R^{2c};
R^{2c} is

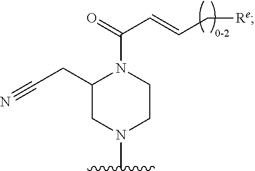

R^{e} is R^{e1}, R^{e2} or R^{e3};
R^{e1} is a 4-10 membered heterocycle which is substituted with 0, 1, 2, 3 or 4 substituents independently selected from halo, hydroxy, C₁-C₄ alkyl, C₁-C₆ aminoalkyl, 4-6 membered heterocycle, —C(O)C₁-C₆ alkyl, C₁-C₆ alkoxy, C₁-C₆ alkoxyalkoxy, C₁-C₄ haloalkyl, C₁-C₄ haloalkoxy and C₂-C₃ alkynyl;
R^{e2} is a 5-6 membered heteroaryl wherein the attachment point is a carbon atom group and wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl;
R^{e3} is —NR⁵R⁶, wherein each R⁵ and R⁶ is independently selected from C₁-C₄ alkyl, C₁-C₆ alkoxy and —CH₂-(4-6 membered heterocycle); and
R^{f} is selected from the group consisting of H, halo, C₁-C₄ alkyl, C₁-C₄ alkoxy, C₁-C₄ haloalkyl and C₁-C₄ haloalkoxy.

In one embodiment, the compound is selected from the group consisting of:

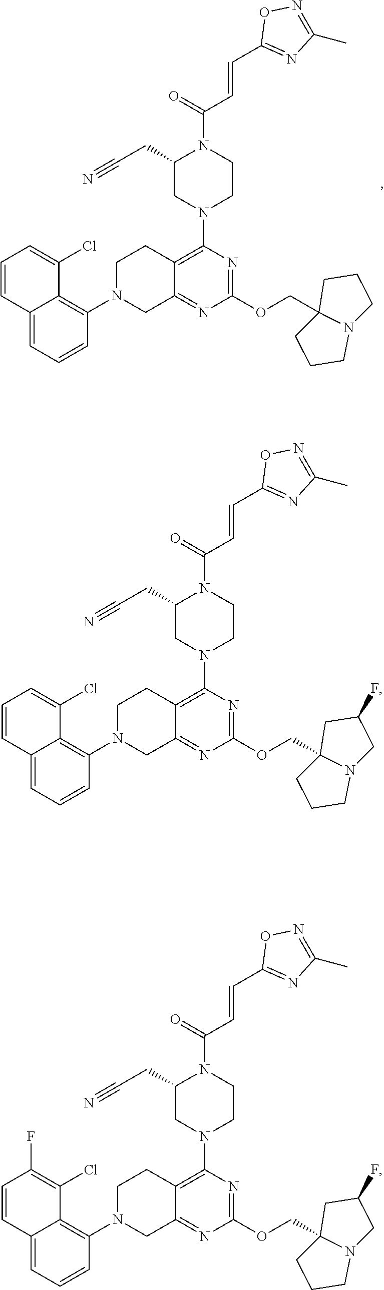 [SMILES: Cc1noc(/C=C/C(=O)N2CCN(c3nc(OCC45CCCN4CCC5)nc4c3CCN(c3cccc5cccc(Cl)c35)C4)C[C@@H]2CC#N)n1 ; Cc1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1 ; Cc1noc(/C=C/C(=O)N2CCN(c3nc(OC[C@@]45CCCN4C[C@H](F)C5)nc4c3CCN(c3cccc5ccc(F)c(Cl)c35)C4)C[C@@H]2CC#N)n1]

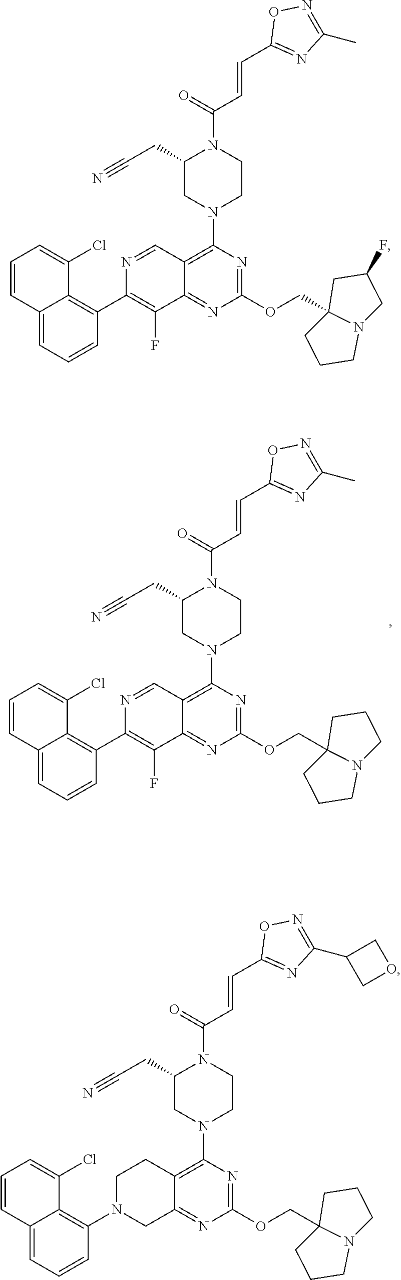 [SMILES: Cc1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; Cc1noc(/C=C/C(=O)N2CCN(c3nc(OCC45CCCN4CCC5)nc4c(F)c(-c5cccc6cccc(Cl)c56)ncc34)C[C@@H]2CC#N)n1 ; N#CC[C@H]1CN(c2nc(OCC34CCCN3CCC4)nc3c2CCN(c2cccc4cccc(Cl)c24)C3)CCN1C(=O)/C=C/c1nc(C2COC2)no1]

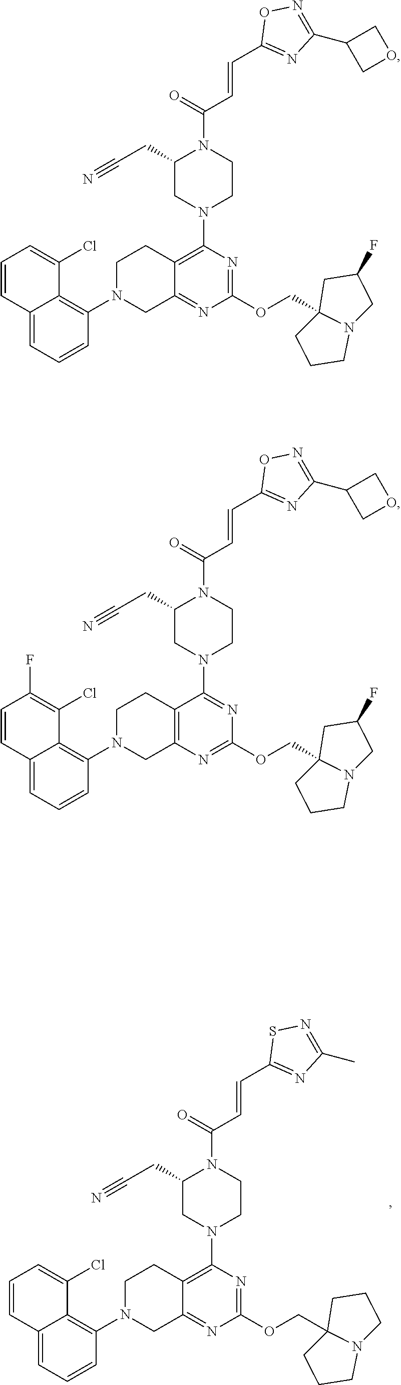 [SMILES: N#CC[C@H]1CN(c2nc(OC[C@@]34CCCN3C[C@H](F)C4)nc3c2CCN(c2cccc4cccc(Cl)c24)C3)CCN1C(=O)/C=C/c1nc(C2COC2)no1 ; N#CC[C@H]1CN(c2nc(OC[C@@]34CCCN3C[C@H](F)C4)nc3c2CCN(c2cccc4ccc(F)c(Cl)c24)C3)CCN1C(=O)/C=C/c1nc(C2COC2)no1 ; Cc1nsc(/C=C/C(=O)N2CCN(c3nc(OCC45CCCN4CCC5)nc4c3CCN(c3cccc5cccc(Cl)c35)C4)C[C@@H]2CC#N)n1]

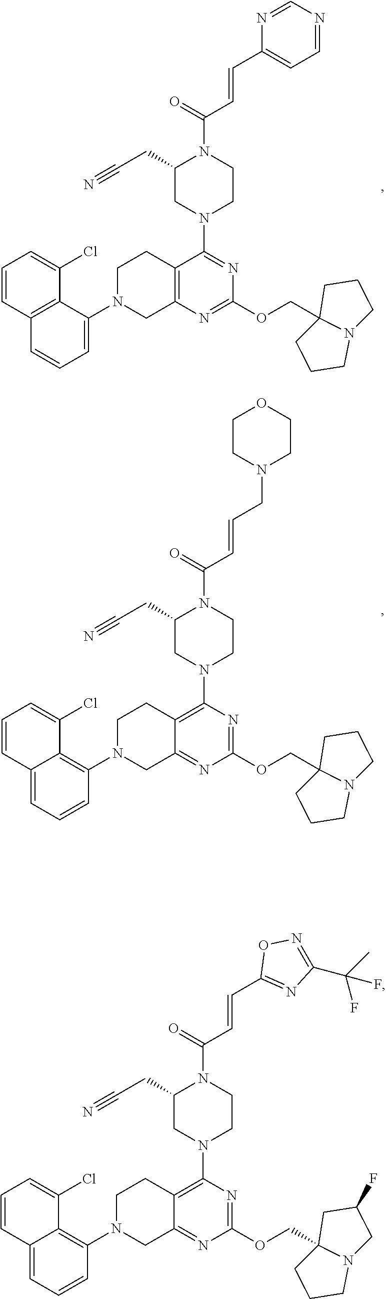 [SMILES: N#CC[C@H]1CN(c2nc(OCC34CCCN3CCC4)nc3c2CCN(c2cccc4cccc(Cl)c24)C3)CCN1C(=O)/C=C/c1ccncn1 ; N#CC[C@H]1CN(c2nc(OCC34CCCN3CCC4)nc3c2CCN(c2cccc4cccc(Cl)c24)C3)CCN1C(=O)/C=C/CN1CCOCC1 ; CC(F)(F)c1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1]

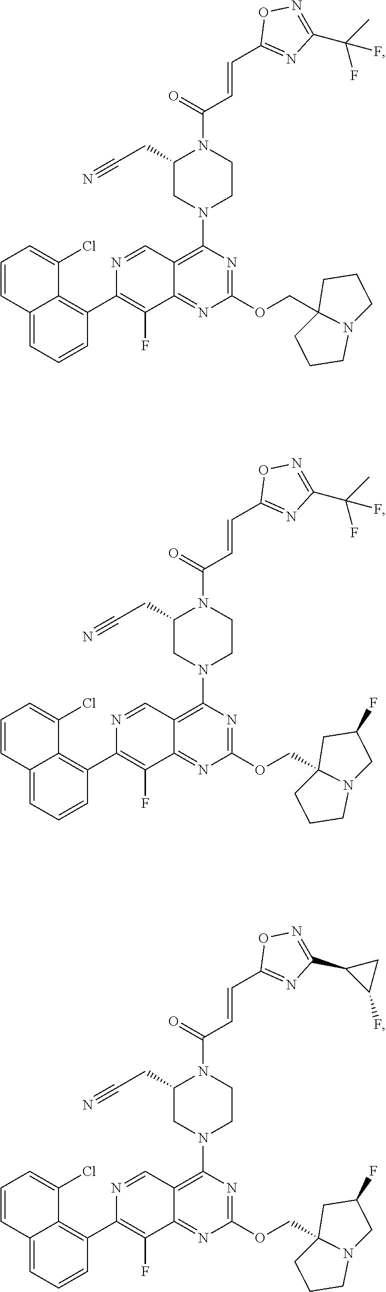 [SMILES: CC(F)(F)c1noc(/C=C/C(=O)N2CCN(c3nc(OCC45CCCN4CCC5)nc4c(F)c(-c5cccc6cccc(Cl)c56)ncc34)C[C@@H]2CC#N)n1 ; CC(F)(F)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/c1nc([C@H]2C[C@@H]2F)no1]

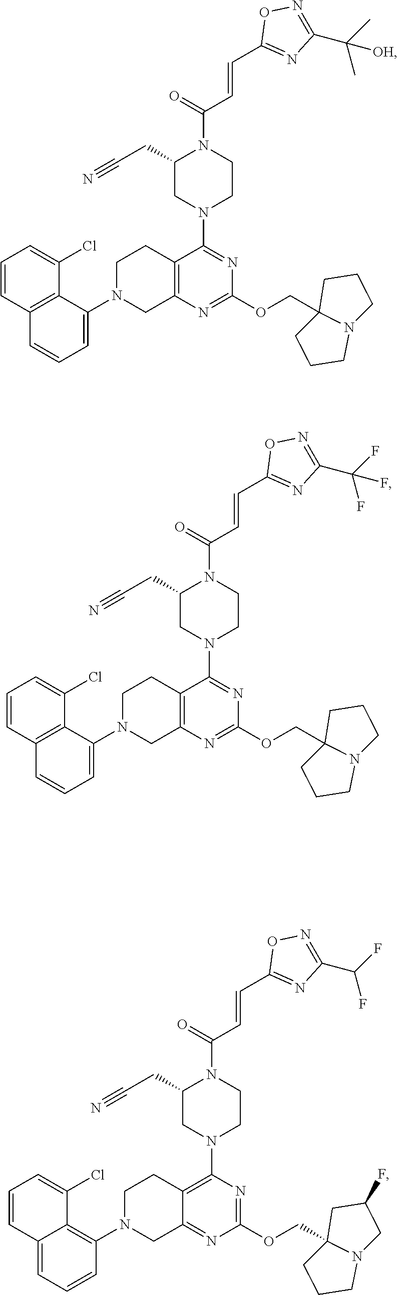 [SMILES: CC(C)(O)c1noc(/C=C/C(=O)N2CCN(c3nc(OCC45CCCN4CCC5)nc4c3CCN(c3cccc5cccc(Cl)c35)C4)C[C@@H]2CC#N)n1 ; N#CC[C@H]1CN(c2nc(OCC34CCCN3CCC4)nc3c2CCN(c2cccc4cccc(Cl)c24)C3)CCN1C(=O)/C=C/c1nc(C(F)(F)F)no1 ; N#CC[C@H]1CN(c2nc(OC[C@@]34CCCN3C[C@H](F)C4)nc3c2CCN(c2cccc4cccc(Cl)c24)C3)CCN1C(=O)/C=C/c1nc(C(F)F)no1]

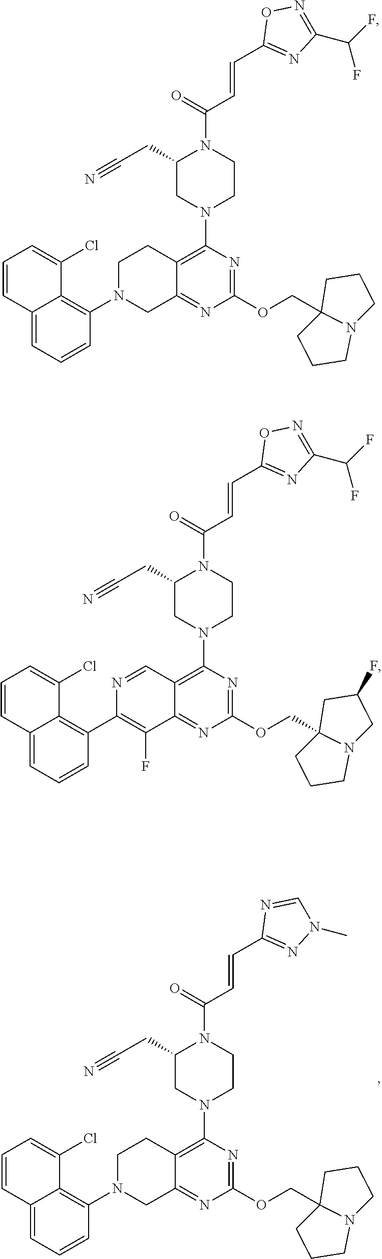 [SMILES: N#CC[C@H]1CN(c2nc(OCC34CCCN3CCC4)nc3c2CCN(c2cccc4cccc(Cl)c24)C3)CCN1C(=O)/C=C/c1nc(C(F)F)no1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/c1nc(C(F)F)no1 ; Cn1cnc(/C=C/C(=O)N2CCN(c3nc(OCC45CCCN4CCC5)nc4c3CCN(c3cccc5cccc(Cl)c35)C4)C[C@@H]2CC#N)n1]

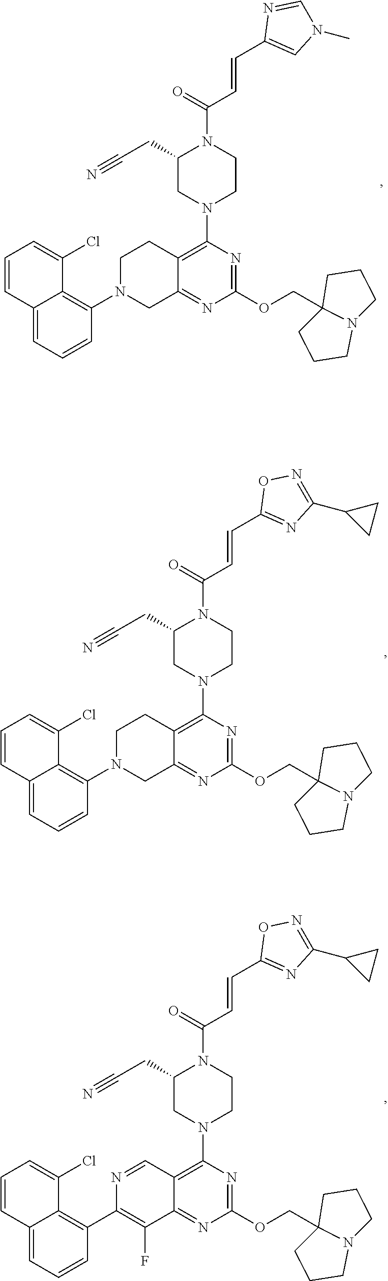 [SMILES: Cn1cnc(/C=C/C(=O)N2CCN(c3nc(OCC45CCCN4CCC5)nc4c3CCN(c3cccc5cccc(Cl)c35)C4)C[C@@H]2CC#N)c1 ; N#CC[C@H]1CN(c2nc(OCC34CCCN3CCC4)nc3c2CCN(c2cccc4cccc(Cl)c24)C3)CCN1C(=O)/C=C/c1nc(C2CC2)no1 ; N#CC[C@H]1CN(c2nc(OCC34CCCN3CCC4)nc3c(F)c(-c4cccc5cccc(Cl)c45)ncc23)CCN1C(=O)/C=C/c1nc(C2CC2)no1]

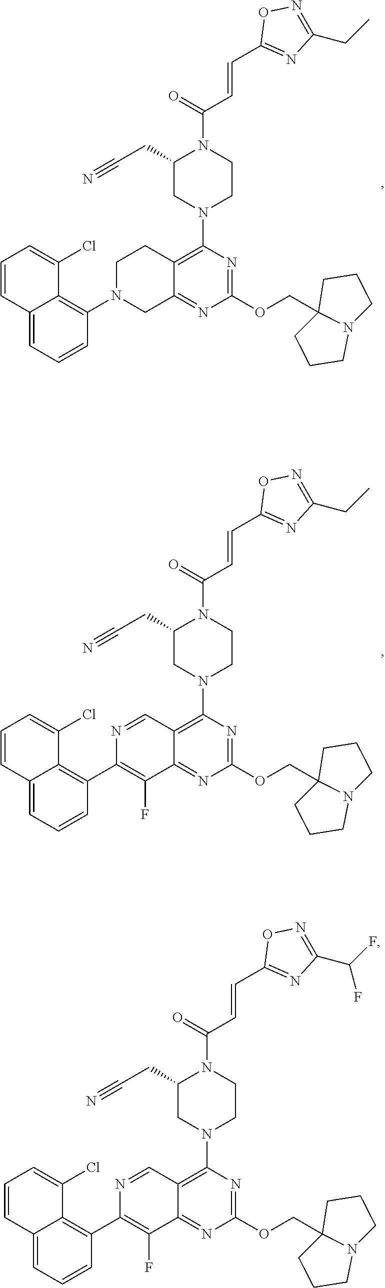 [SMILES: CCc1noc(/C=C/C(=O)N2CCN(c3nc(OCC45CCCN4CCC5)nc4c3CCN(c3cccc5cccc(Cl)c35)C4)C[C@@H]2CC#N)n1 ; CCc1noc(/C=C/C(=O)N2CCN(c3nc(OCC45CCCN4CCC5)nc4c(F)c(-c5cccc6cccc(Cl)c56)ncc34)C[C@@H]2CC#N)n1 ; N#CC[C@H]1CN(c2nc(OCC34CCCN3CCC4)nc3c(F)c(-c4cccc5cccc(Cl)c45)ncc23)CCN1C(=O)/C=C/c1nc(C(F)F)no1]

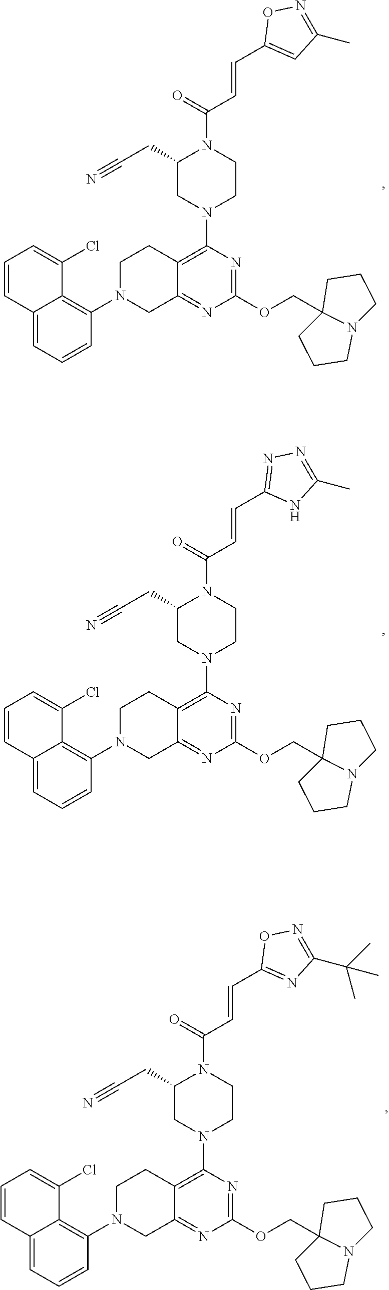 [SMILES: Cc1cc(/C=C/C(=O)N2CCN(c3nc(OCC45CCCN4CCC5)nc4c3CCN(c3cccc5cccc(Cl)c35)C4)C[C@@H]2CC#N)on1 ; Cc1nnc(/C=C/C(=O)N2CCN(c3nc(OCC45CCCN4CCC5)nc4c3CCN(c3cccc5cccc(Cl)c35)C4)C[C@@H]2CC#N)[nH]1 ; CC(C)(C)c1noc(/C=C/C(=O)N2CCN(c3nc(OCC45CCCN4CCC5)nc4c3CCN(c3cccc5cccc(Cl)c35)C4)C[C@@H]2CC#N)n1]

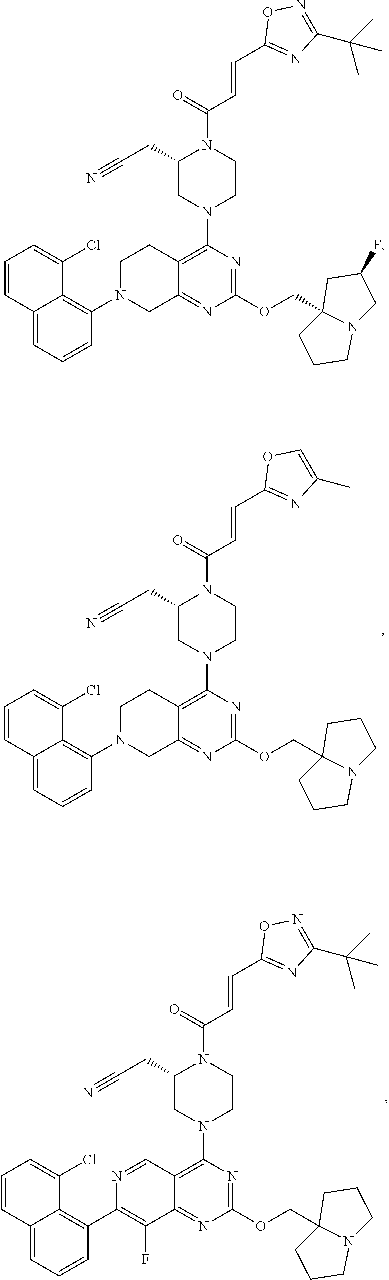 [SMILES: CC(C)(C)c1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1 ; Cc1coc(/C=C/C(=O)N2CCN(c3nc(OCC45CCCN4CCC5)nc4c3CCN(c3cccc5cccc(Cl)c35)C4)C[C@@H]2CC#N)n1 ; CC(C)(C)c1noc(/C=C/C(=O)N2CCN(c3nc(OCC45CCCN4CCC5)nc4c(F)c(-c5cccc6cccc(Cl)c56)ncc34)C[C@@H]2CC#N)n1]

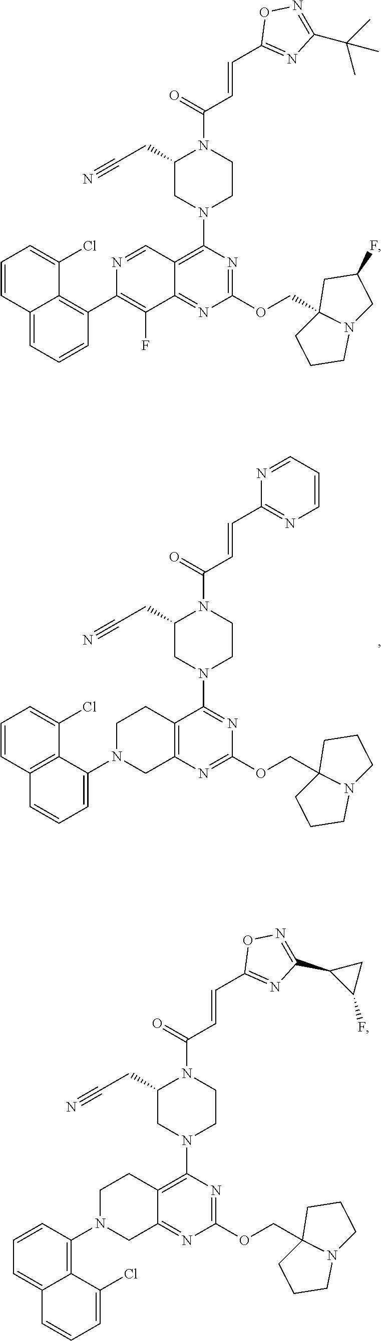 [SMILES: CC(C)(C)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; N#CC[C@H]1CN(c2nc(OCC34CCCN3CCC4)nc3c2CCN(c2cccc4cccc(Cl)c24)C3)CCN1C(=O)/C=C/c1ncccn1 ; N#CC[C@H]1CN(c2nc(OCC34CCCN3CCC4)nc3c2CCN(c2cccc4cccc(Cl)c24)C3)CCN1C(=O)/C=C/c1nc([C@H]2C[C@@H]2F)no1]

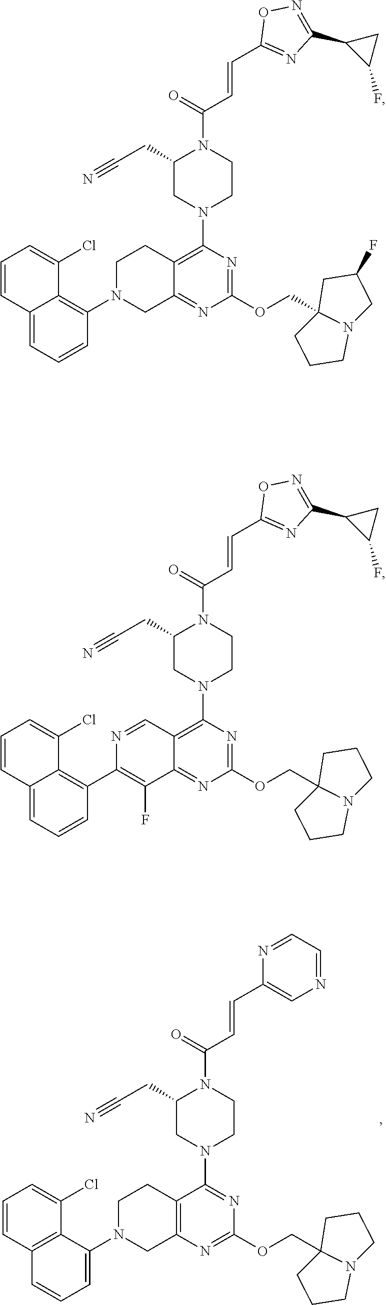 [SMILES: N#CC[C@H]1CN(c2nc(OC[C@@]34CCCN3C[C@H](F)C4)nc3c2CCN(c2cccc4cccc(Cl)c24)C3)CCN1C(=O)/C=C/c1nc([C@H]2C[C@@H]2F)no1 ; N#CC[C@H]1CN(c2nc(OCC34CCCN3CCC4)nc3c(F)c(-c4cccc5cccc(Cl)c45)ncc23)CCN1C(=O)/C=C/c1nc([C@H]2C[C@@H]2F)no1 ; N#CC[C@H]1CN(c2nc(OCC34CCCN3CCC4)nc3c2CCN(c2cccc4cccc(Cl)c24)C3)CCN1C(=O)/C=C/c1cnccn1]

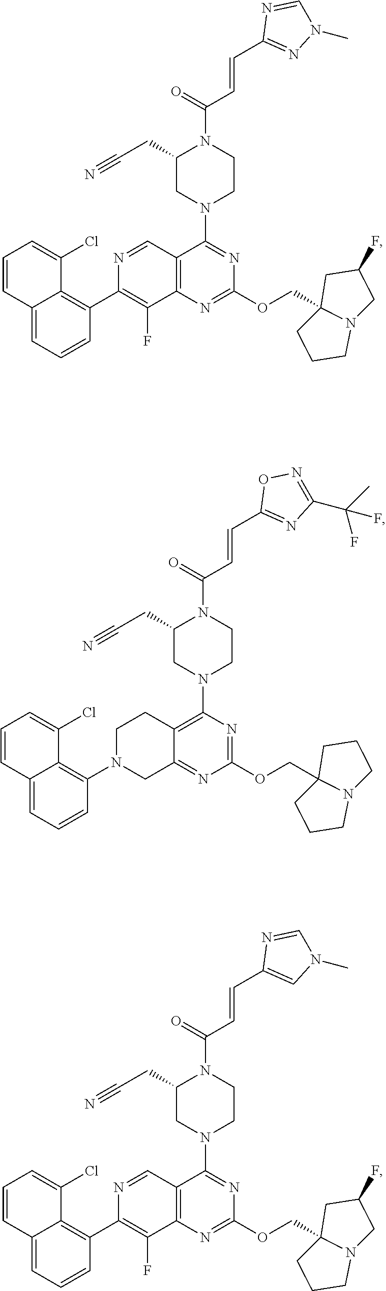 [SMILES: Cn1cnc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; CC(F)(F)c1noc(/C=C/C(=O)N2CCN(c3nc(OCC45CCCN4CCC5)nc4c3CCN(c3cccc5cccc(Cl)c35)C4)C[C@@H]2CC#N)n1 ; Cn1cnc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)c1]

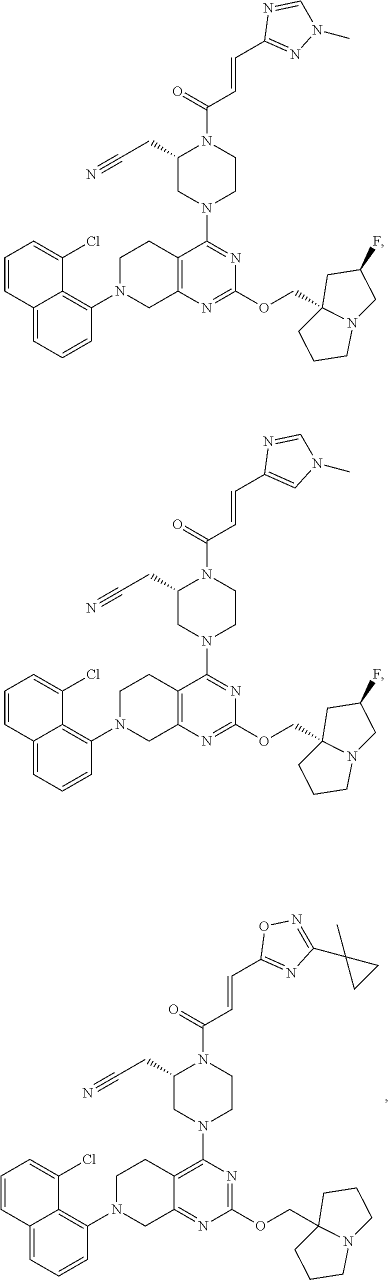 [SMILES: Cn1cnc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1 ; Cn1cnc(/C=C/C(=O)N2CCN(c3nc(OC[C@@]45CCCN4C[C@H](F)C5)nc4c3CCN(c3cccc5cccc(Cl)c35)C4)C[C@@H]2CC#N)c1 ; CC1(c2noc(/C=C/C(=O)N3CCN(c4nc(OCC56CCCN5CCC6)nc5c4CCN(c4cccc6cccc(Cl)c46)C5)C[C@@H]3CC#N)n2)CC1]

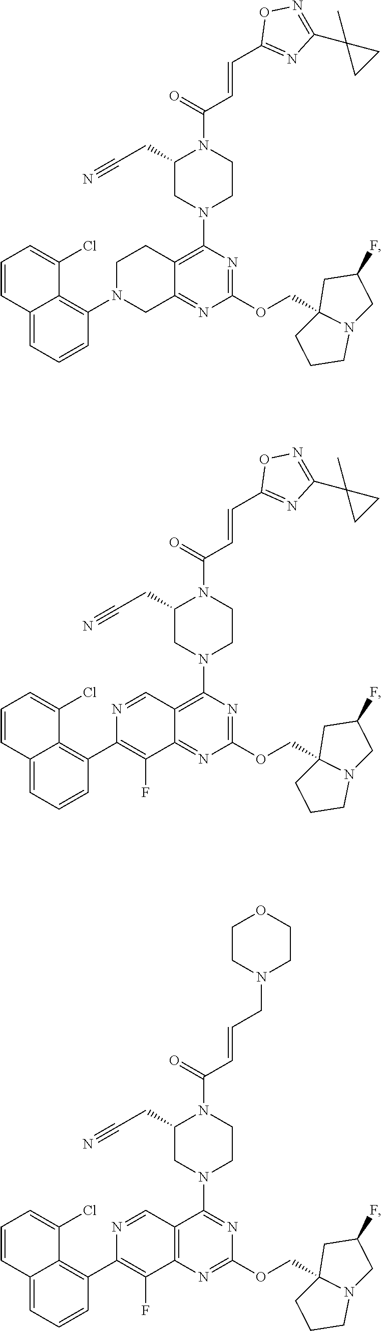 [SMILES: CC1(c2noc(/C=C/C(=O)N3CCN(c4nc(OC[C@@]56CCCN5C[C@H](F)C6)nc5c4CCN(c4cccc6cccc(Cl)c46)C5)C[C@@H]3CC#N)n2)CC1 ; CC1(c2noc(/C=C/C(=O)N3CCN(c4nc(OC[C@@]56CCCN5C[C@H](F)C6)nc5c(F)c(-c6cccc7cccc(Cl)c67)ncc45)C[C@@H]3CC#N)n2)CC1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCOCC1]

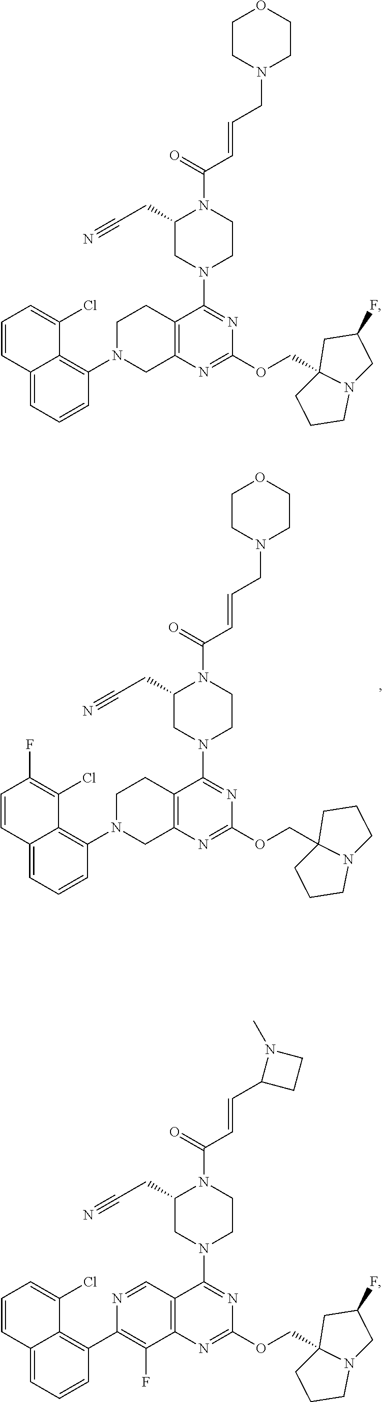 [SMILES: N#CC[C@H]1CN(c2nc(OC[C@@]34CCCN3C[C@H](F)C4)nc3c2CCN(c2cccc4cccc(Cl)c24)C3)CCN1C(=O)/C=C/CN1CCOCC1 ; N#CC[C@H]1CN(c2nc(OCC34CCCN3CCC4)nc3c2CCN(c2cccc4ccc(F)c(Cl)c24)C3)CCN1C(=O)/C=C/CN1CCOCC1 ; CN1CCC1/C=C/C(=O)N1CCN(c2nc(OC[C@@]34CCCN3C[C@H](F)C4)nc3c(F)c(-c4cccc5cccc(Cl)c45)ncc23)C[C@@H]1CC#N]

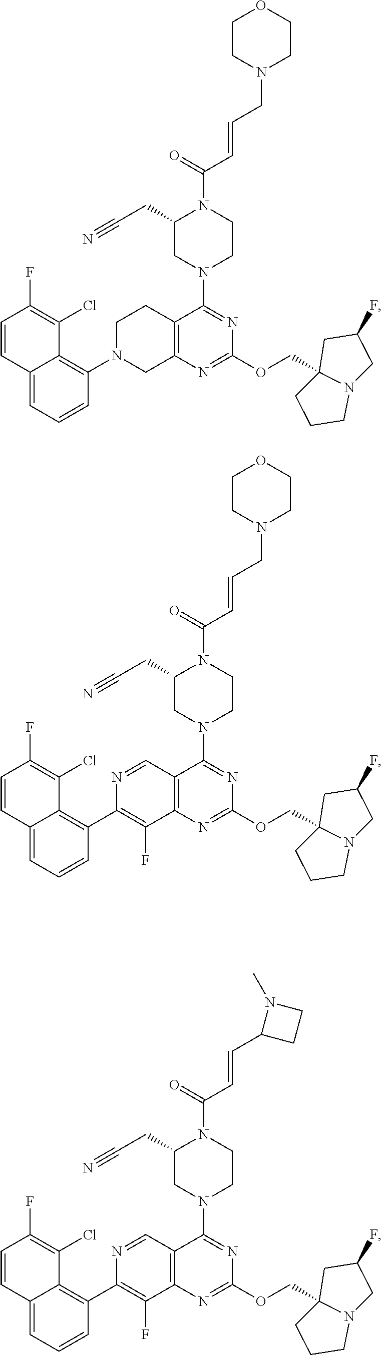 [SMILES: N#CC[C@H]1CN(c2nc(OC[C@@]34CCCN3C[C@H](F)C4)nc3c2CCN(c2cccc4ccc(F)c(Cl)c24)C3)CCN1C(=O)/C=C/CN1CCOCC1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCOCC1 ; CN1CCC1/C=C/C(=O)N1CCN(c2nc(OC[C@@]34CCCN3C[C@H](F)C4)nc3c(F)c(-c4cccc5ccc(F)c(Cl)c45)ncc23)C[C@@H]1CC#N]

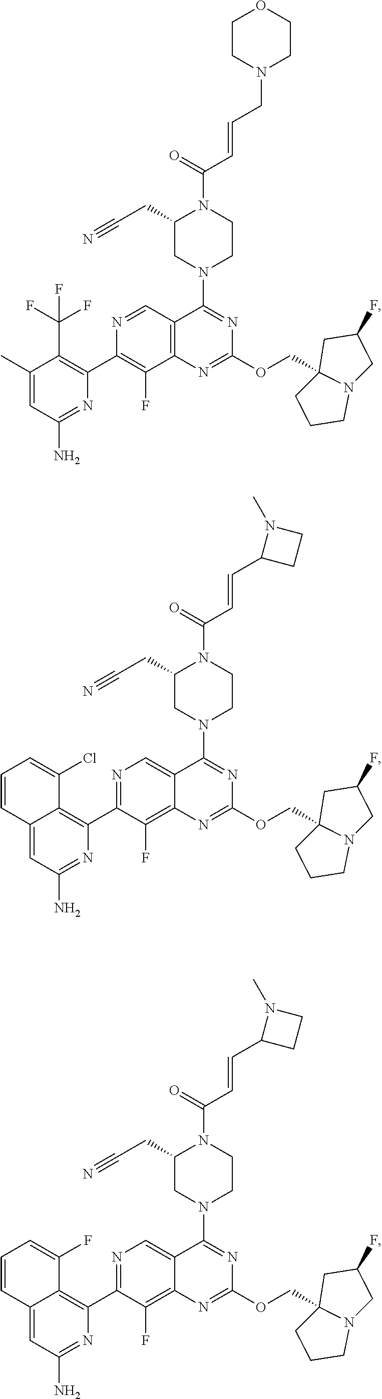 [SMILES: Cc1cc(N)nc(-c2ncc3c(N4CCN(C(=O)/C=C/CN5CCOCC5)[C@@H](CC#N)C4)nc(OC[C@@]45CCCN4C[C@H](F)C5)nc3c2F)c1C(F)(F)F ; CN1CCC1/C=C/C(=O)N1CCN(c2nc(OC[C@@]34CCCN3C[C@H](F)C4)nc3c(F)c(-c4nc(N)cc5cccc(Cl)c45)ncc23)C[C@@H]1CC#N ; CN1CCC1/C=C/C(=O)N1CCN(c2nc(OC[C@@]34CCCN3C[C@H](F)C4)nc3c(F)c(-c4nc(N)cc5cccc(F)c45)ncc23)C[C@@H]1CC#N]

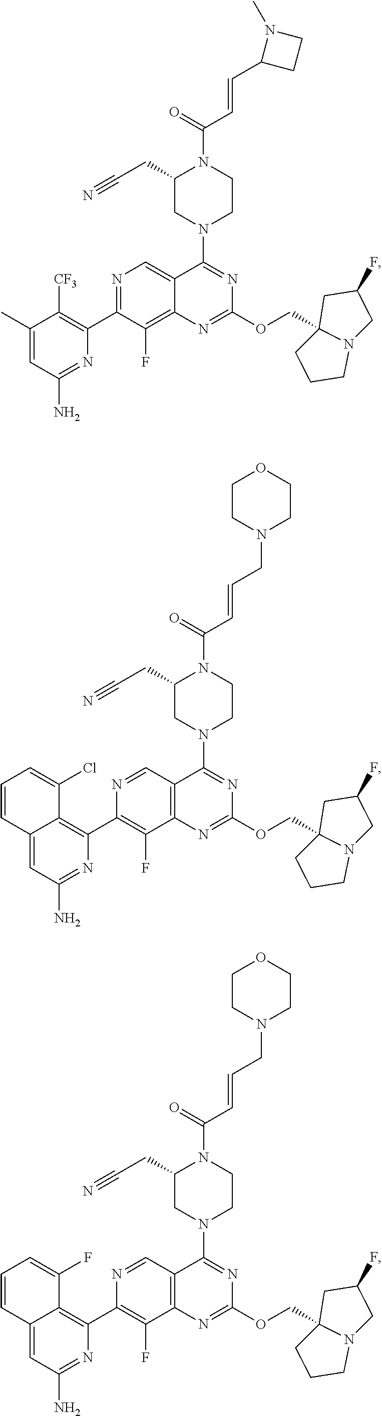 [SMILES: Cc1cc(N)nc(-c2ncc3c(N4CCN(C(=O)/C=C/C5CCN5C)[C@@H](CC#N)C4)nc(OC[C@@]45CCCN4C[C@H](F)C5)nc3c2F)c1C(F)(F)F ; N#CC[C@H]1CN(c2nc(OC[C@@]34CCCN3C[C@H](F)C4)nc3c(F)c(-c4nc(N)cc5cccc(Cl)c45)ncc23)CCN1C(=O)/C=C/CN1CCOCC1 ; N#CC[C@H]1CN(c2nc(OC[C@@]34CCCN3C[C@H](F)C4)nc3c(F)c(-c4nc(N)cc5cccc(F)c45)ncc23)CCN1C(=O)/C=C/CN1CCOCC1]

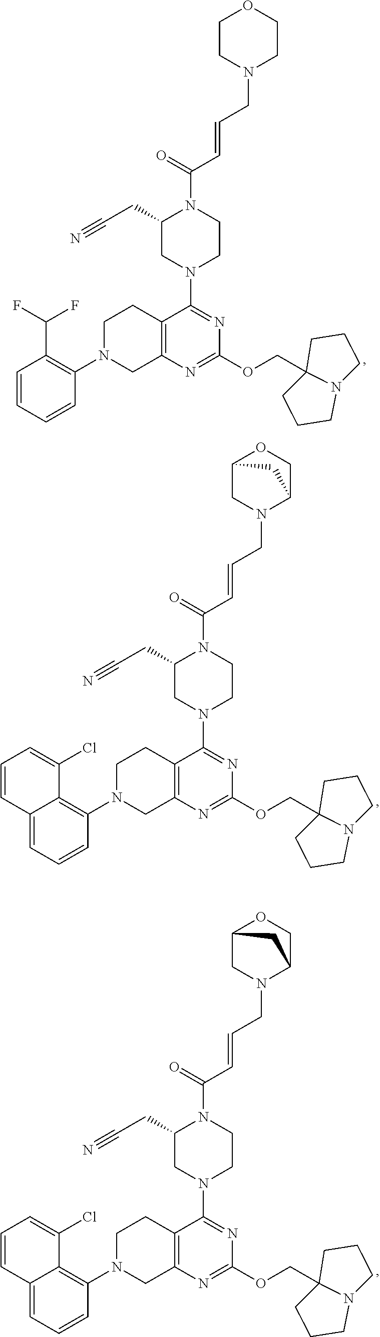

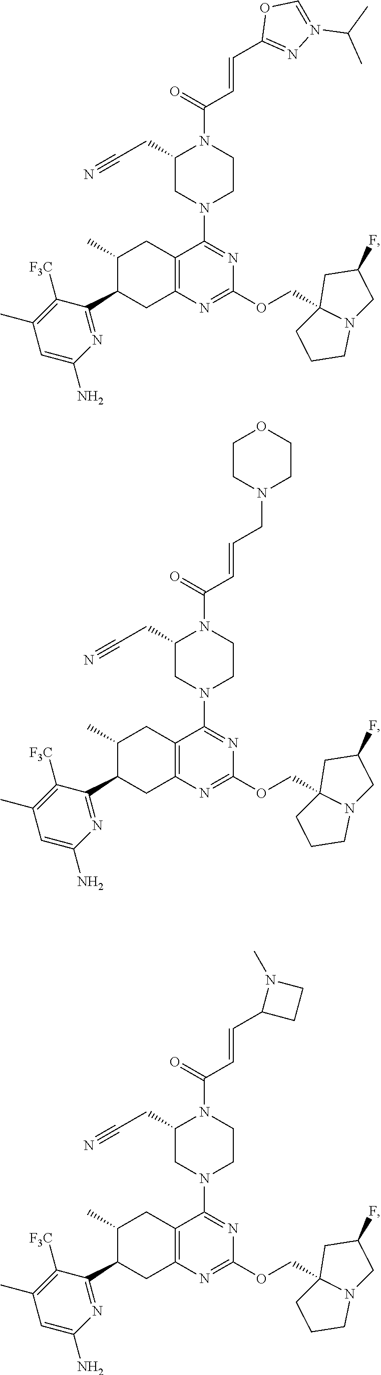

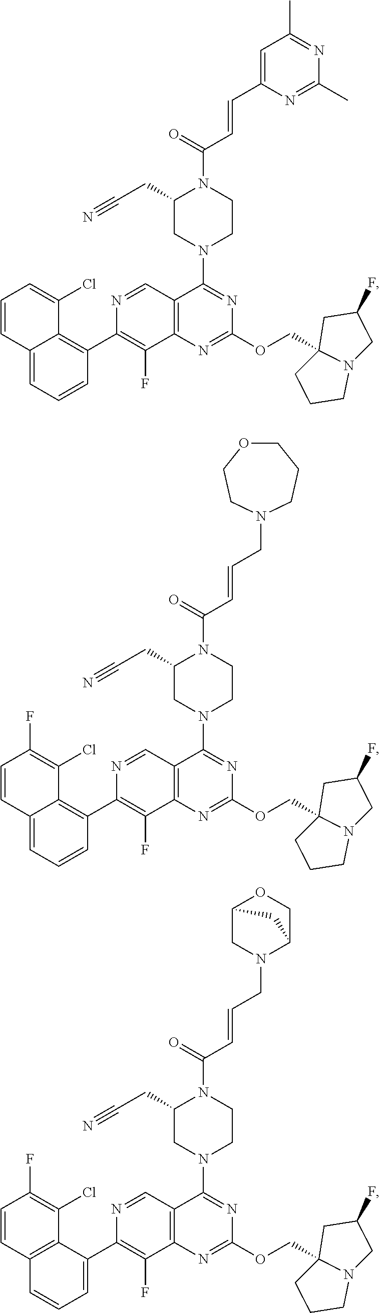

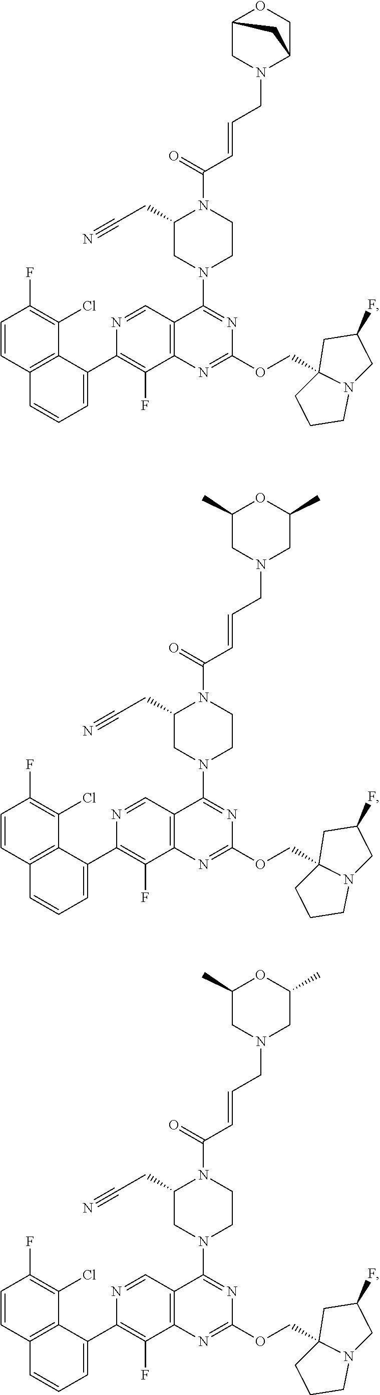 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@H]2C[C@@H]1CO2 ; C[C@@H]1CN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C[C@H](C)O1 ; C[C@@H]1CN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C[C@@H](C)O1]

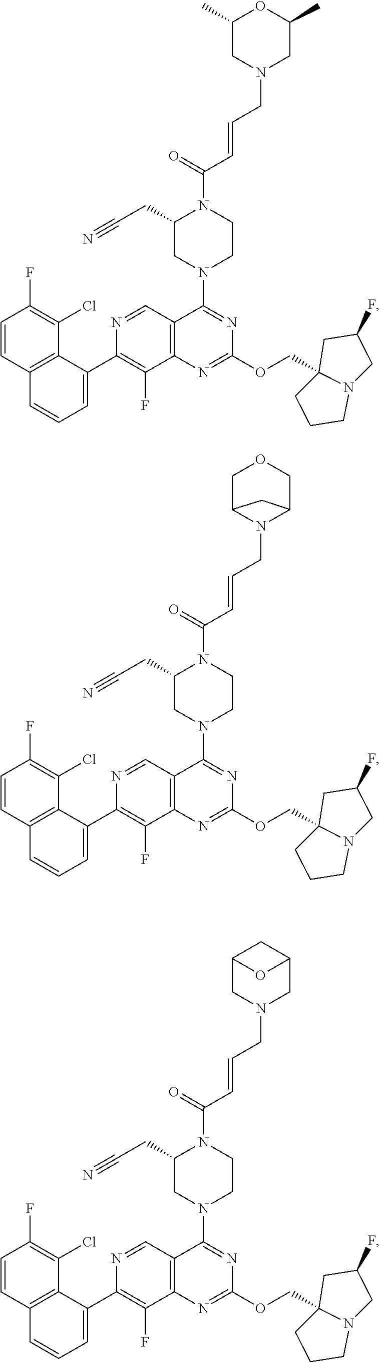 [SMILES: C[C@H]1CN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C[C@H](C)O1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C2COCC1C2 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2CC(C1)O2]

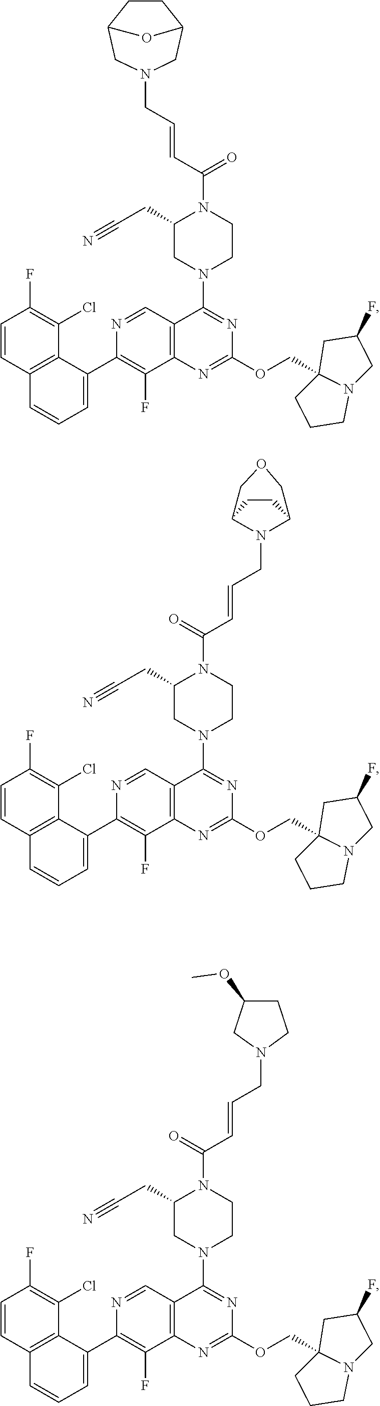 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2CCC(C1)O2 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1[C@@H]2CC[C@H]1COC2 ; CO[C@H]1CCN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C1]

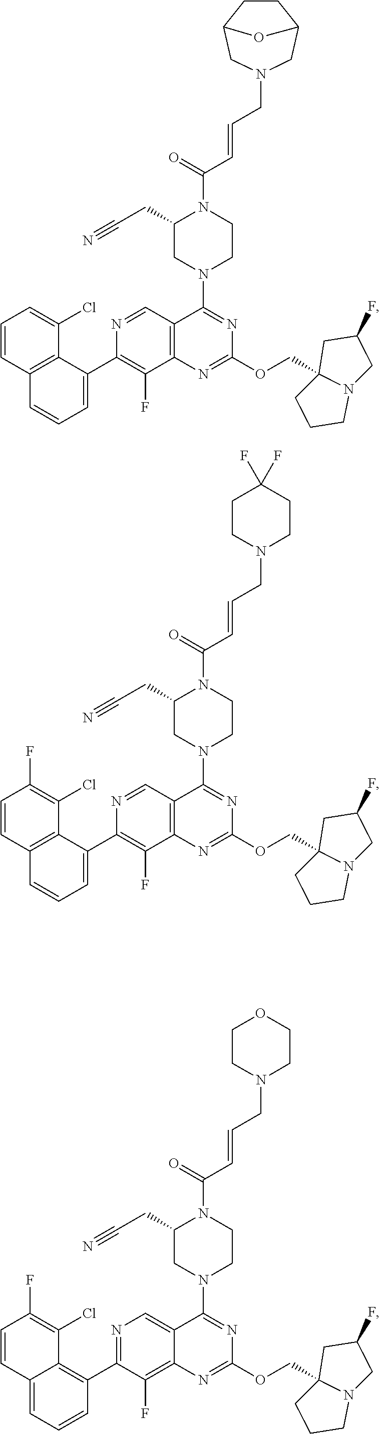

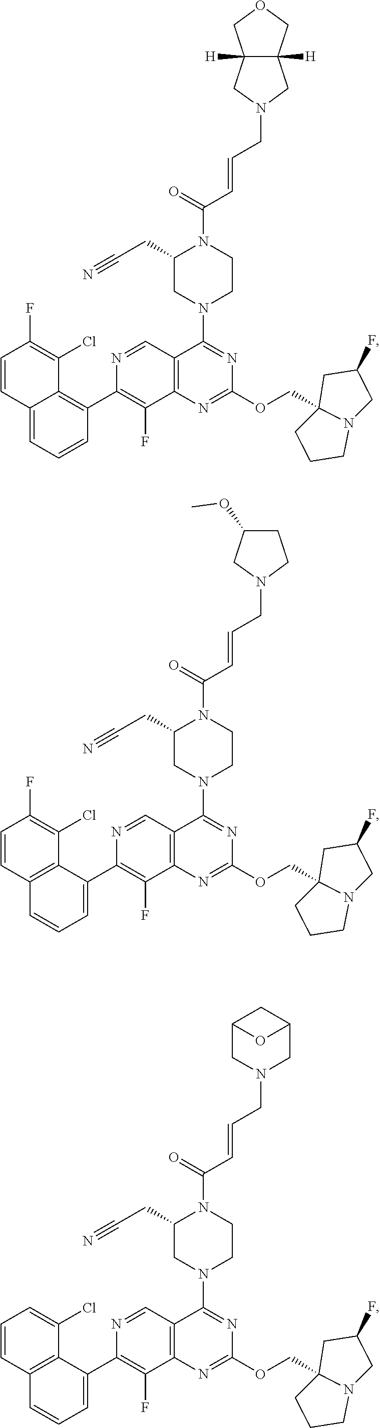 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@H]2COC[C@H]2C1 ; CO[C@@H]1CCN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CC(C1)O2]

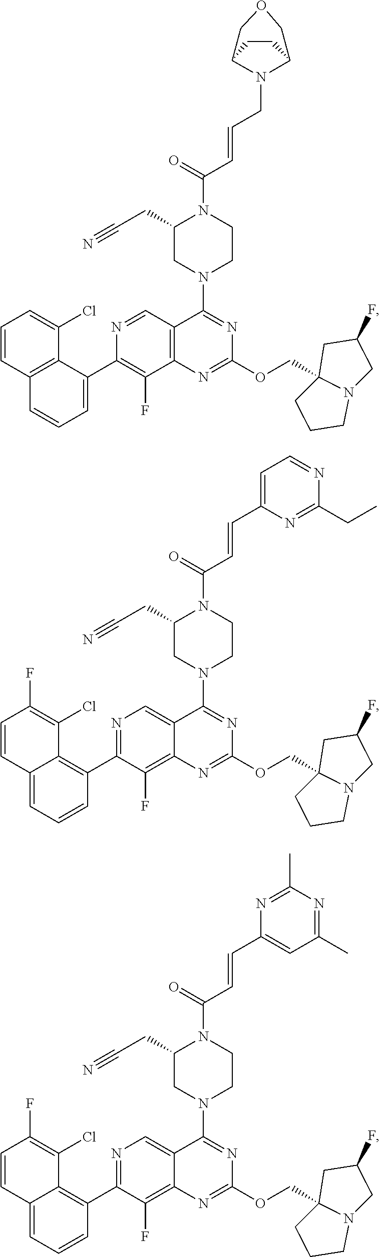

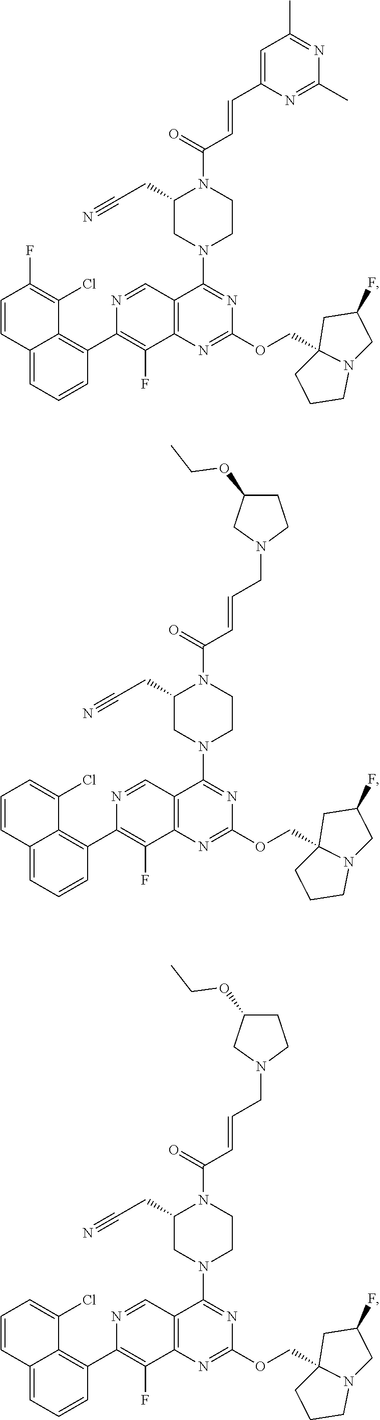 [SMILES: Cc1cc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)nc(C)n1 ; CCO[C@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1 ; CCO[C@@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1]

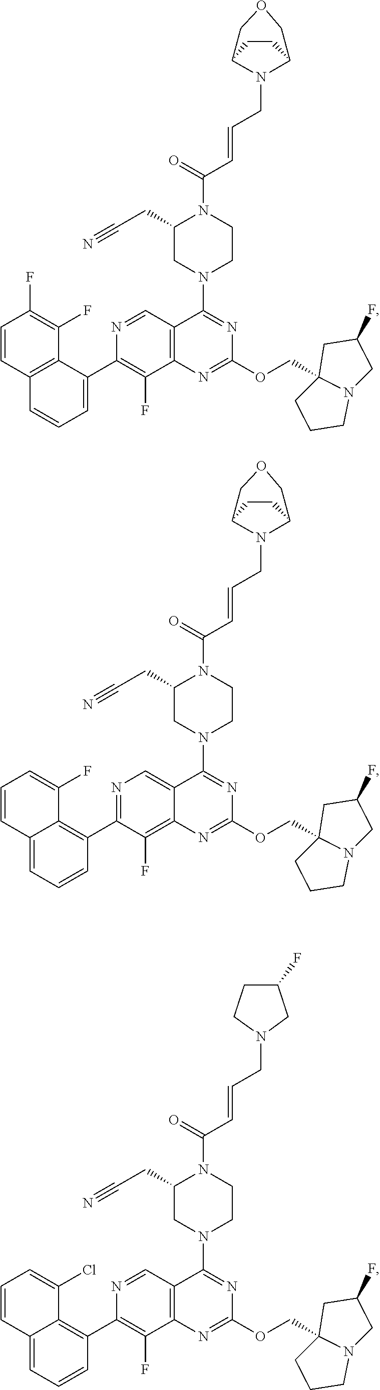 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1[C@@H]2CC[C@H]1COC2 ; N#CC[C@H]1CN(c2nc(OC[C@@]34CCCN3C[C@H](F)C4)nc3c(F)c(-c4cccc5cccc(F)c45)ncc23)CCN1C(=O)/C=C/CN1[C@@H]2CC[C@H]1COC2 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@H](F)C1]

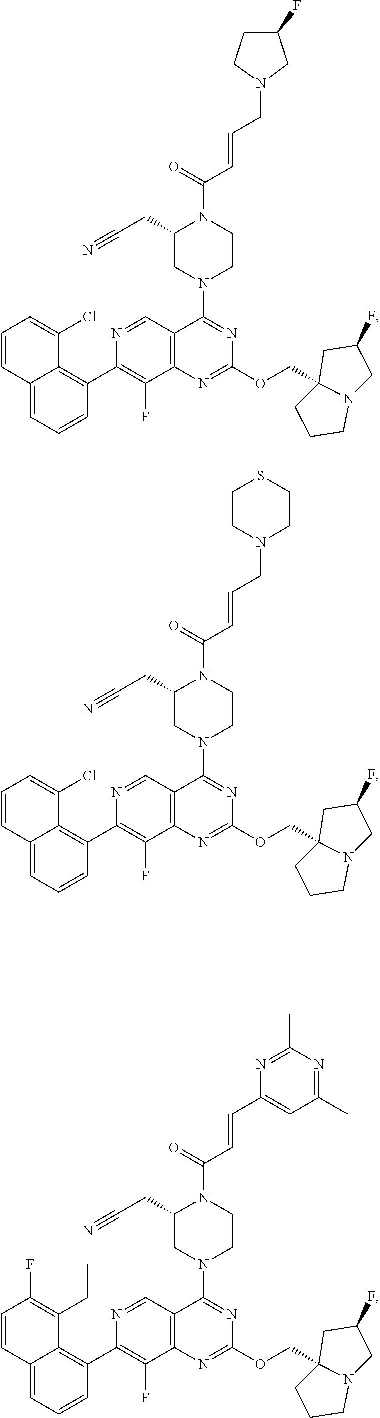 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@@H](F)C1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCSCC1 ; CCc1c(F)ccc2cccc(-c3ncc4c(N5CCN(C(=O)/C=C/c6cc(C)nc(C)n6)[C@@H](CC#N)C5)nc(OC[C@@]56CCCN5C[C@H](F)C6)nc4c3F)c12]

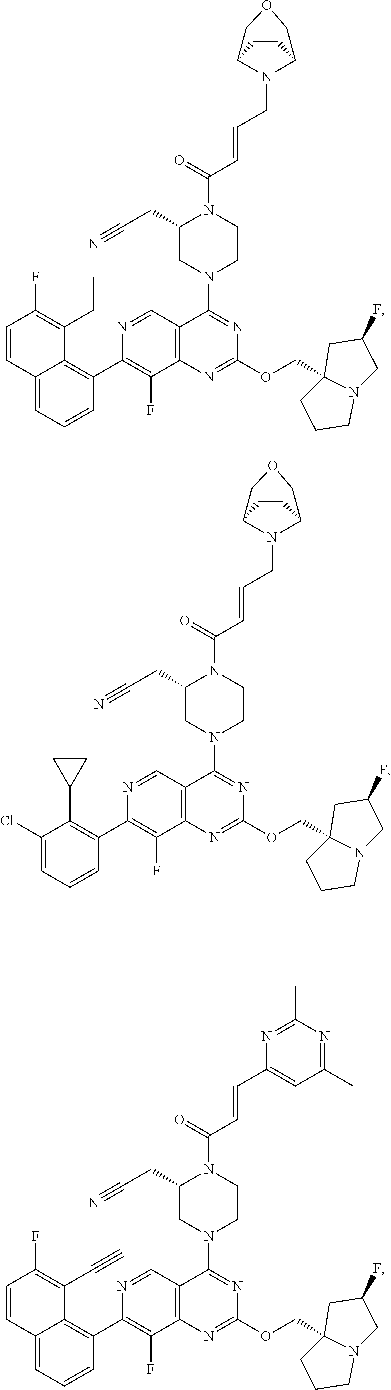 [SMILES: CCc1c(F)ccc2cccc(-c3ncc4c(N5CCN(C(=O)/C=C/CN6[C@@H]7CC[C@H]6COC7)[C@@H](CC#N)C5)nc(OC[C@@]56CCCN5C[C@H](F)C6)nc4c3F)c12 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1[C@@H]2CC[C@H]1COC2 ; C#Cc1c(F)ccc2cccc(-c3ncc4c(N5CCN(C(=O)/C=C/c6cc(C)nc(C)n6)[C@@H](CC#N)C5)nc(OC[C@@]56CCCN5C[C@H](F)C6)nc4c3F)c12]

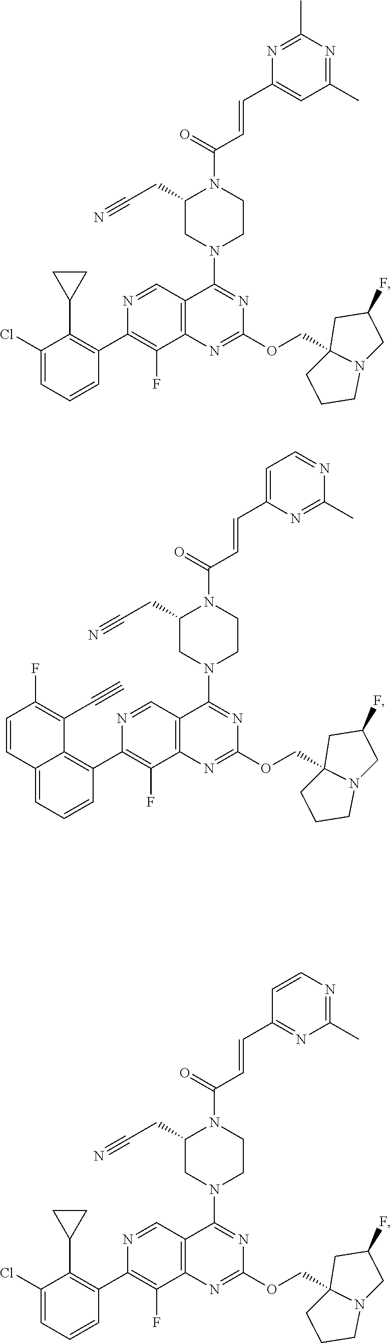 [SMILES: Cc1cc(/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)nc(C)n1 ; C#Cc1c(F)ccc2cccc(-c3ncc4c(N5CCN(C(=O)/C=C/c6ccnc(C)n6)[C@@H](CC#N)C5)nc(OC[C@@]56CCCN5C[C@H](F)C6)nc4c3F)c12 ; Cc1nccc(/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)n1]

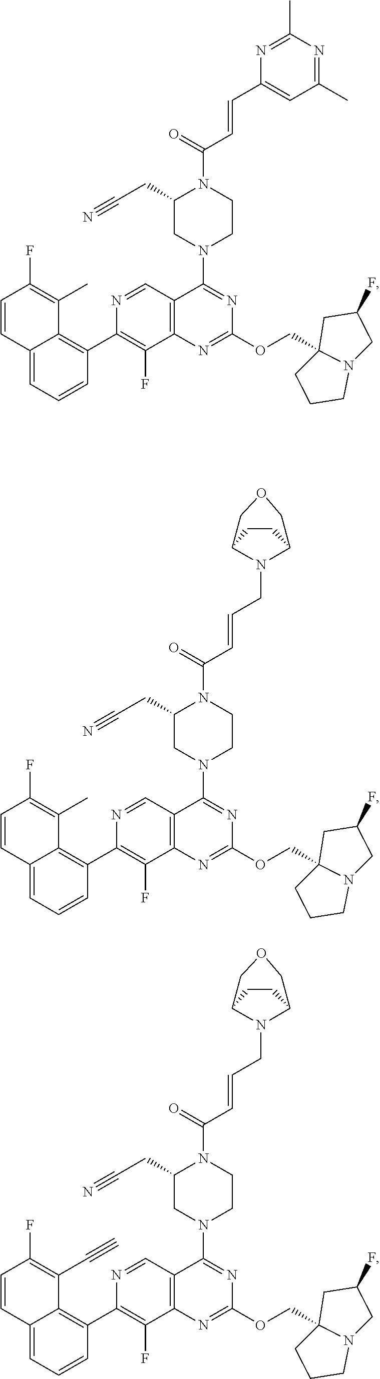 [SMILES: Cc1cc(/C=C/C(=O)N2CCN(c3nc(OC[C@@]45CCCN4C[C@H](F)C5)nc4c(F)c(-c5cccc6ccc(F)c(C)c56)ncc34)C[C@@H]2CC#N)nc(C)n1 ; Cc1c(F)ccc2cccc(-c3ncc4c(N5CCN(C(=O)/C=C/CN6[C@@H]7CC[C@H]6COC7)[C@@H](CC#N)C5)nc(OC[C@@]56CCCN5C[C@H](F)C6)nc4c3F)c12 ; C#Cc1c(F)ccc2cccc(-c3ncc4c(N5CCN(C(=O)/C=C/CN6[C@@H]7CC[C@H]6COC7)[C@@H](CC#N)C5)nc(OC[C@@]56CCCN5C[C@H](F)C6)nc4c3F)c12]

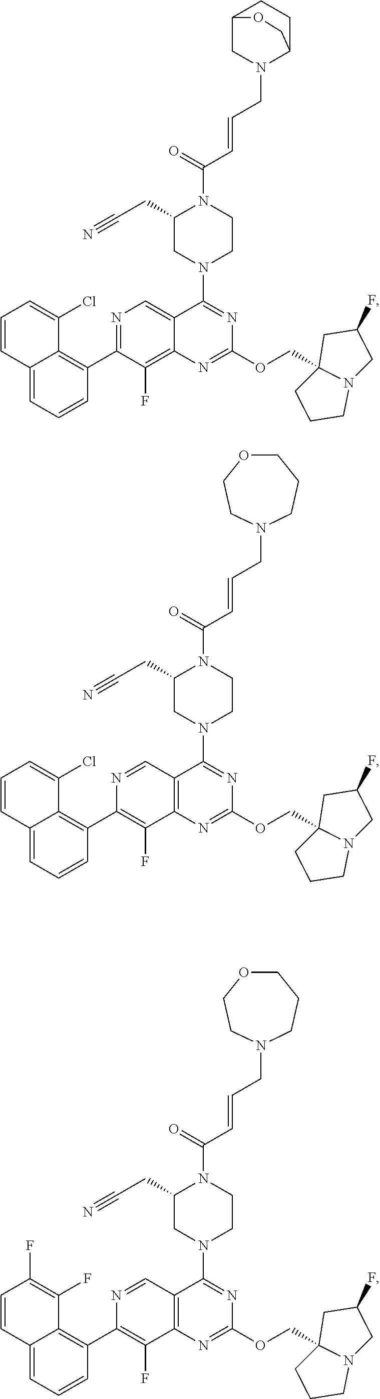 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CCC1CO2 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCCOCC1 ; N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCCOCC1]

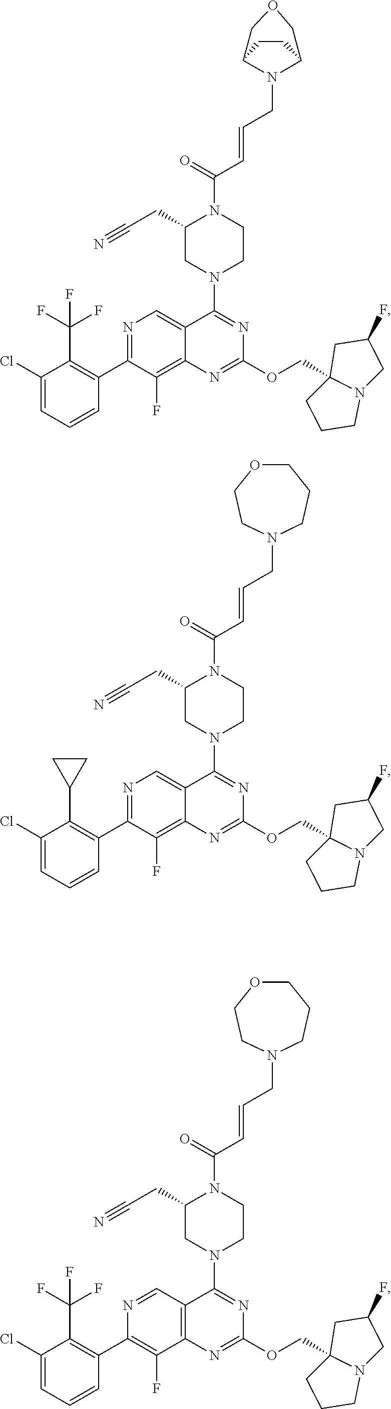 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1[C@@H]2CC[C@H]1COC2 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CCCOCC1 ; N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CCCOCC1]

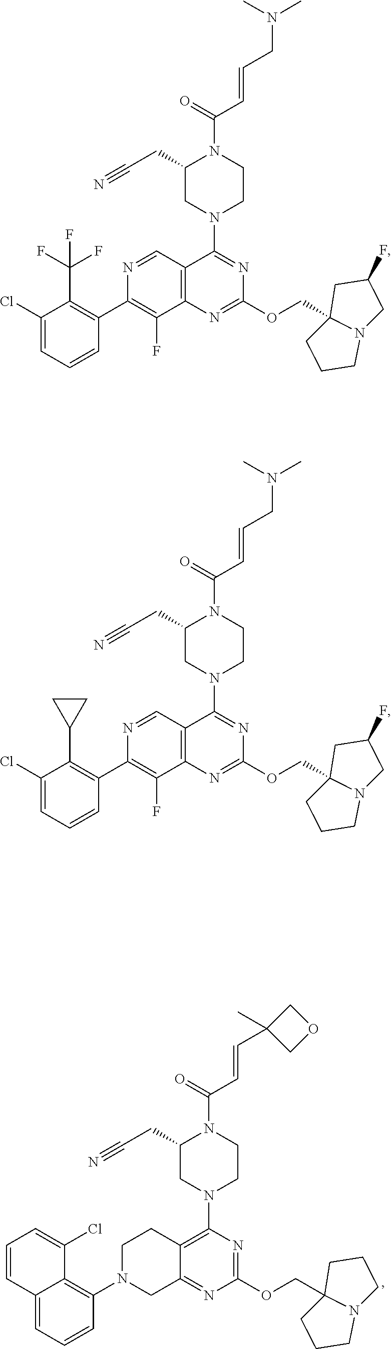 [SMILES: CN(C)C/C=C/C(=O)N1CCN(c2nc(OC[C@@]34CCCN3C[C@H](F)C4)nc3c(F)c(-c4cccc(Cl)c4C(F)(F)F)ncc23)C[C@@H]1CC#N ; CN(C)C/C=C/C(=O)N1CCN(c2nc(OC[C@@]34CCCN3C[C@H](F)C4)nc3c(F)c(-c4cccc(Cl)c4C4CC4)ncc23)C[C@@H]1CC#N ; CC1(/C=C/C(=O)N2CCN(c3nc(OCC45CCCN4CCC5)nc4c3CCN(c3cccc5cccc(Cl)c35)C4)C[C@@H]2CC#N)COC1]

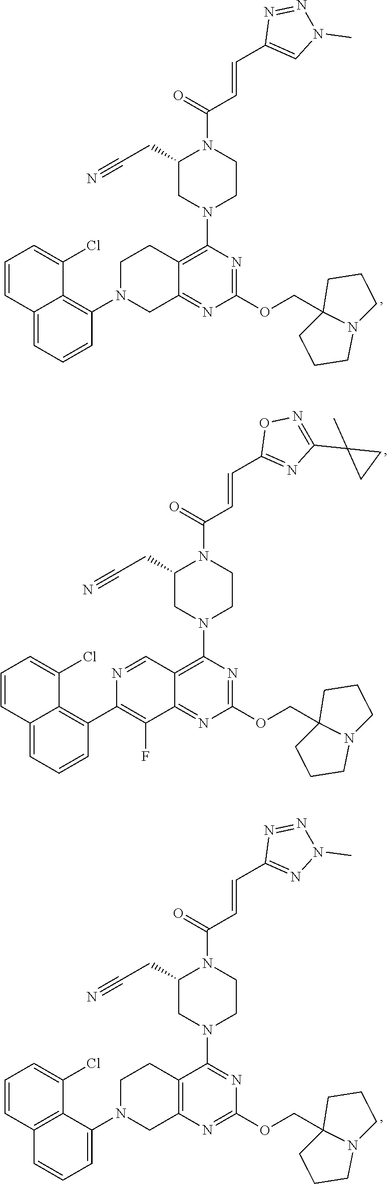 [SMILES: Cn1cc(/C=C/C(=O)N2CCN(c3nc(OCC45CCCN4CCC5)nc4c3CCN(c3cccc5cccc(Cl)c35)C4)C[C@@H]2CC#N)nn1 ; CC1(c2noc(/C=C/C(=O)N3CCN(c4nc(OCC56CCCN5CCC6)nc5c(F)c(-c6cccc7cccc(Cl)c67)ncc45)C[C@@H]3CC#N)n2)CC1 ; Cn1nnc(/C=C/C(=O)N2CCN(c3nc(OCC45CCCN4CCC5)nc4c3CCN(c3cccc5cccc(Cl)c35)C4)C[C@@H]2CC#N)n1]

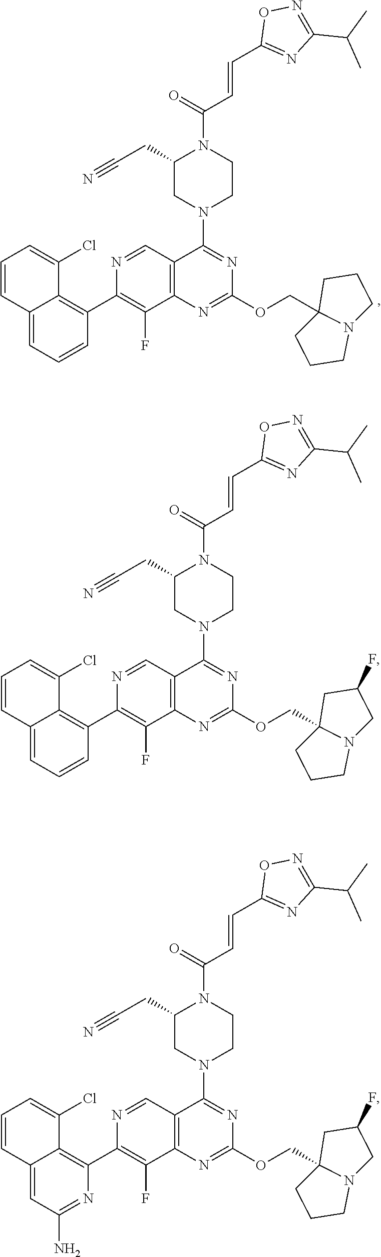 [SMILES: CC(C)c1noc(/C=C/C(=O)N2CCN(c3nc(OCC45CCCN4CCC5)nc4c(F)c(-c5cccc6cccc(Cl)c56)ncc34)C[C@@H]2CC#N)n1 ; CC(C)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; CC(C)c1noc(/C=C/C(=O)N2CCN(c3nc(OC[C@@]45CCCN4C[C@H](F)C5)nc4c(F)c(-c5nc(N)cc6cccc(Cl)c56)ncc34)C[C@@H]2CC#N)n1]

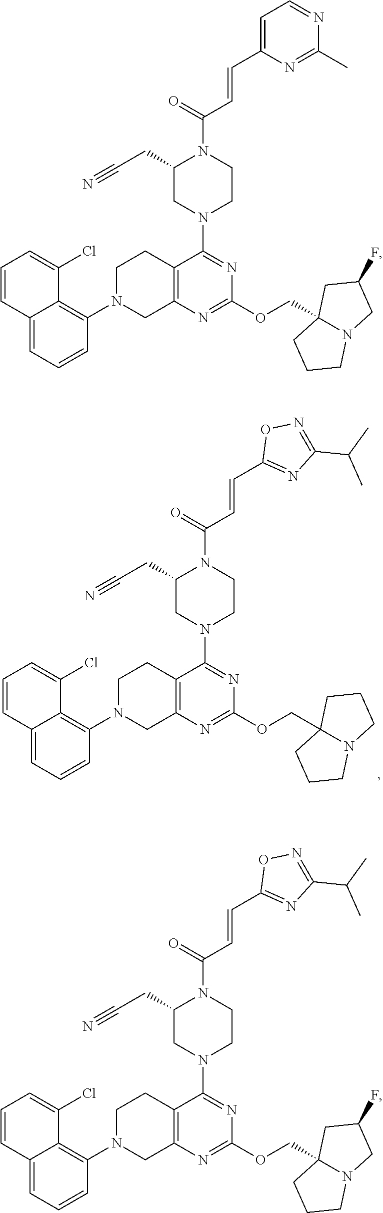 [SMILES: Cc1nccc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1 ; CC(C)c1noc(/C=C/C(=O)N2CCN(c3nc(OCC45CCCN4CCC5)nc4c3CCN(c3cccc5cccc(Cl)c35)C4)C[C@@H]2CC#N)n1 ; CC(C)c1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1]

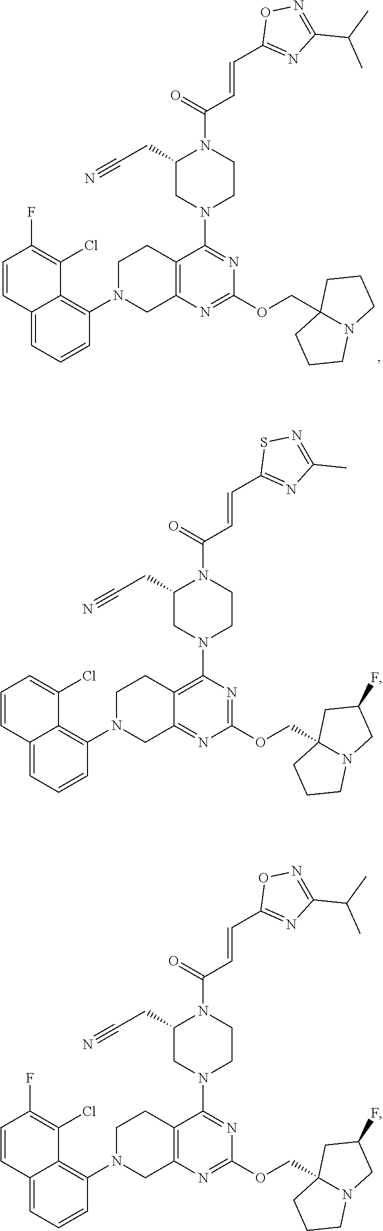 [SMILES: CC(C)c1noc(/C=C/C(=O)N2CCN(c3nc(OCC45CCCN4CCC5)nc4c3CCN(c3cccc5ccc(F)c(Cl)c35)C4)C[C@@H]2CC#N)n1 ; Cc1nsc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1 ; CC(C)c1noc(/C=C/C(=O)N2CCN(c3nc(OC[C@@]45CCCN4C[C@H](F)C5)nc4c3CCN(c3cccc5ccc(F)c(Cl)c35)C4)C[C@@H]2CC#N)n1]

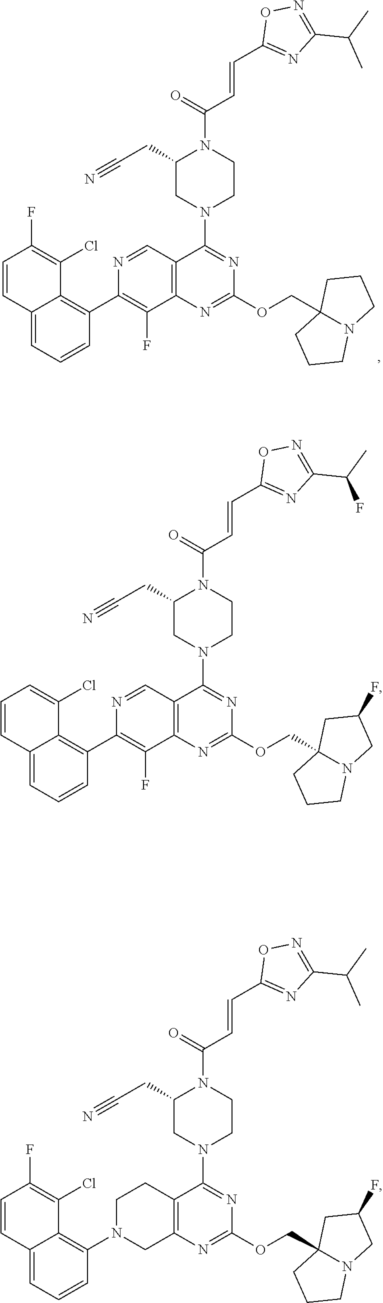 [SMILES: CC(C)c1noc(/C=C/C(=O)N2CCN(c3nc(OCC45CCCN4CCC5)nc4c(F)c(-c5cccc6ccc(F)c(Cl)c56)ncc34)C[C@@H]2CC#N)n1 ; C[C@@H](F)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; CC(C)c1noc(/C=C/C(=O)N2CCN(c3nc(OC[C@]45CCCN4C[C@H](F)C5)nc4c3CCN(c3cccc5ccc(F)c(Cl)c35)C4)C[C@@H]2CC#N)n1]

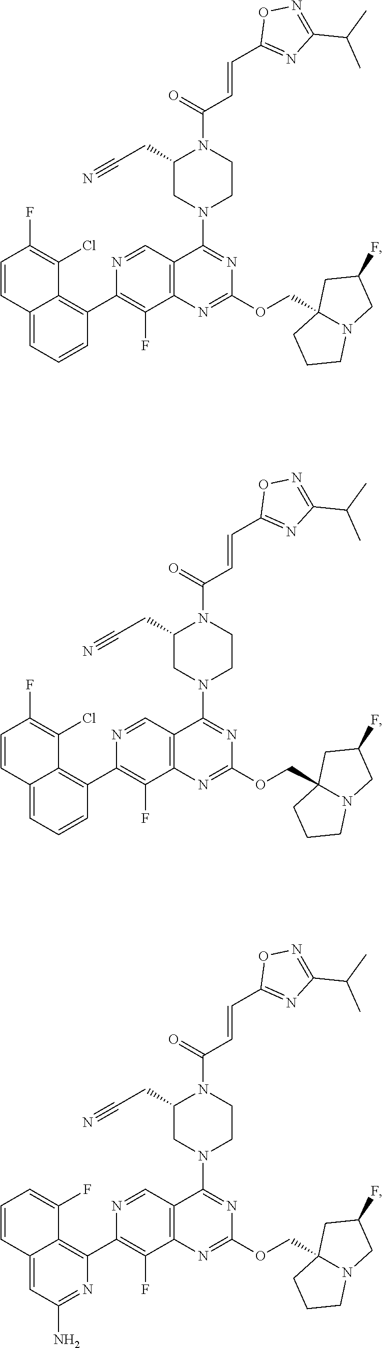 [SMILES: CC(C)c1noc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1 ; CC(C)c1noc(/C=C/C(=O)N2CCN(c3nc(OC[C@]45CCCN4C[C@H](F)C5)nc4c(F)c(-c5cccc6ccc(F)c(Cl)c56)ncc34)C[C@@H]2CC#N)n1 ; CC(C)c1noc(/C=C/C(=O)N2CCN(c3nc(OC[C@@]45CCCN4C[C@H](F)C5)nc4c(F)c(-c5nc(N)cc6cccc(F)c56)ncc34)C[C@@H]2CC#N)n1]

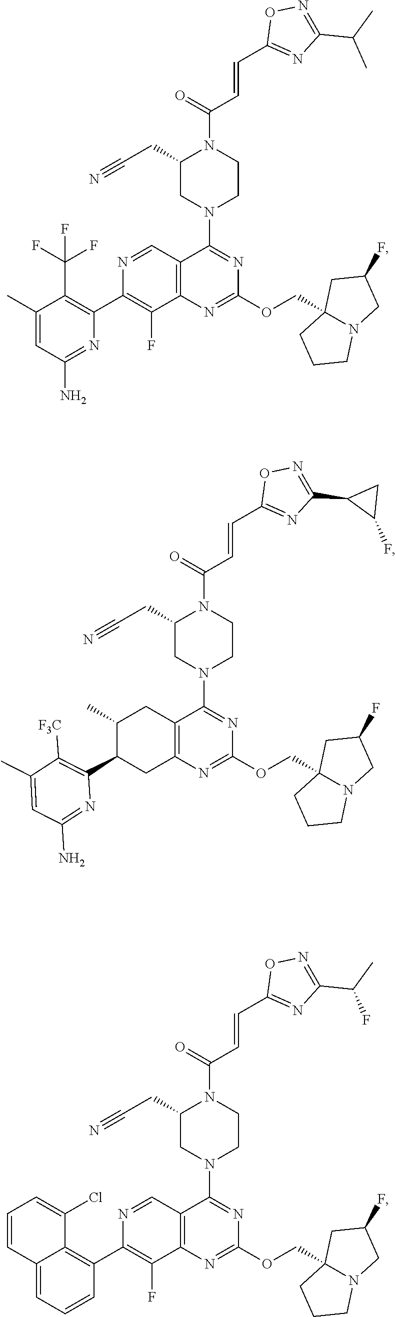 [SMILES: Cc1cc(N)nc(-c2ncc3c(N4CCN(C(=O)/C=C/c5nc(C(C)C)no5)[C@@H](CC#N)C4)nc(OC[C@@]45CCCN4C[C@H](F)C5)nc3c2F)c1C(F)(F)F ; Cc1cc(N)nc([C@@H]2Cc3nc(OC[C@@]45CCCN4C[C@H](F)C5)nc(N4CCN(C(=O)/C=C/c5nc([C@H]6C[C@@H]6F)no5)[C@@H](CC#N)C4)c3C[C@H]2C)c1C(F)(F)F ; C[C@H](F)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1]

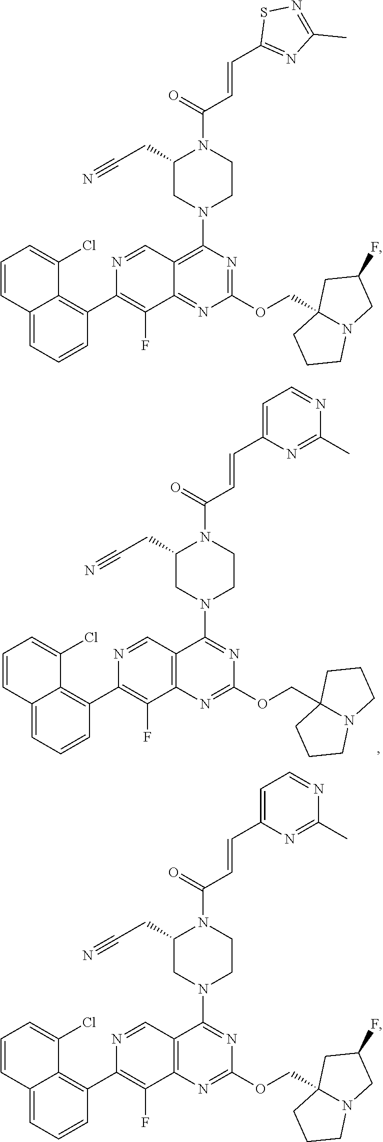

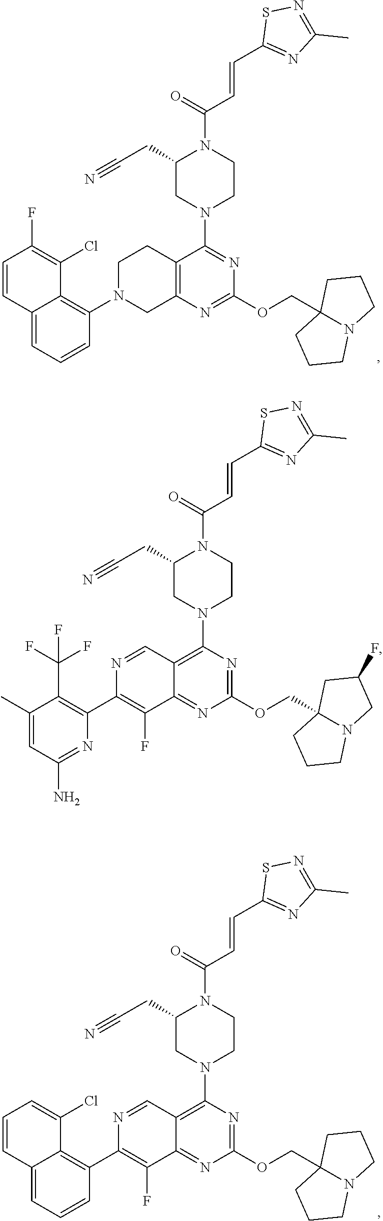

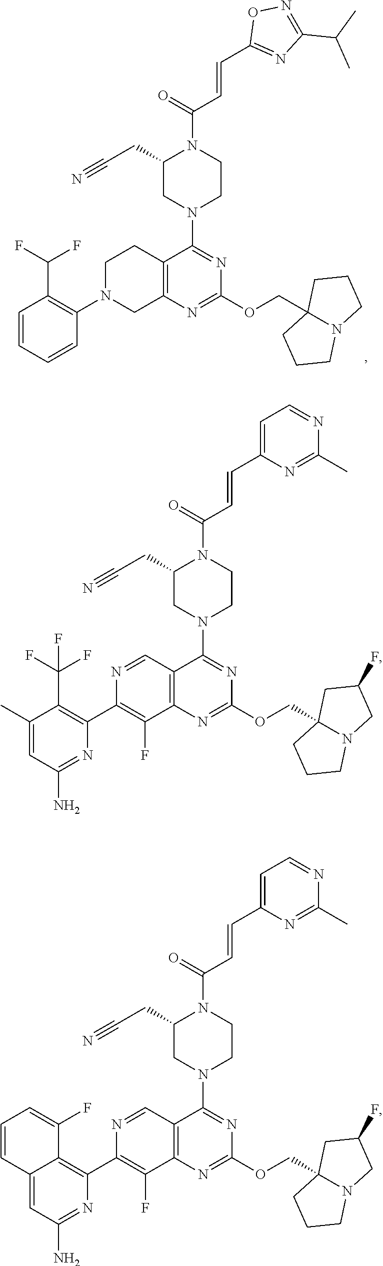

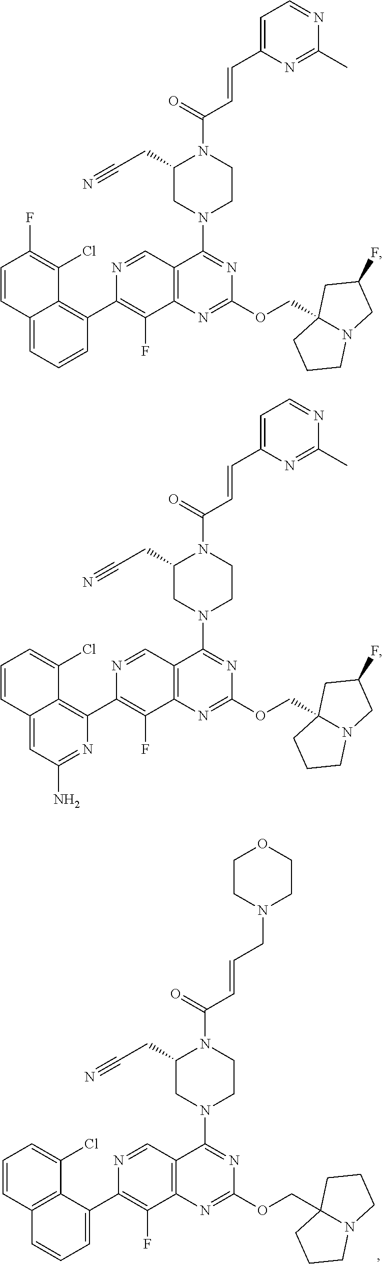

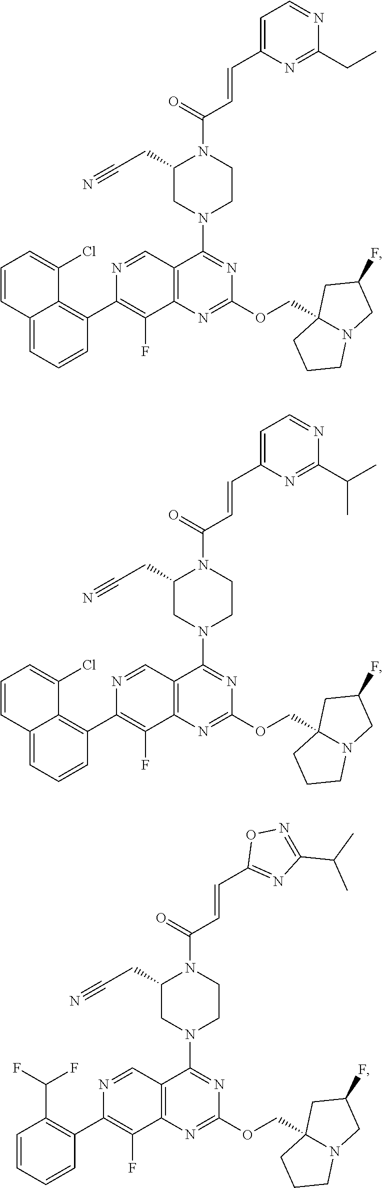 [SMILES: CCc1nccc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; CC(C)c1nccc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; CC(C)c1noc(/C=C/C(=O)N2CCN(c3nc(OC[C@@]45CCCN4C[C@H](F)C5)nc4c(F)c(-c5ccccc5C(F)F)ncc34)C[C@@H]2CC#N)n1]

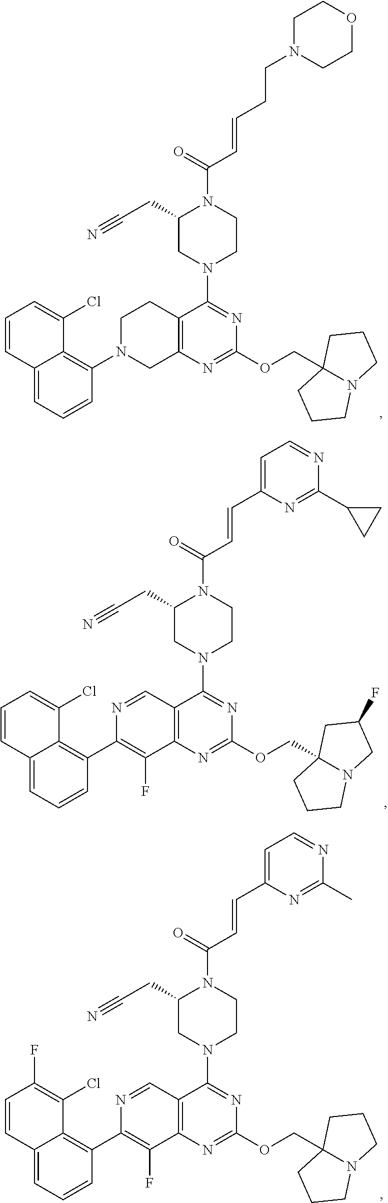

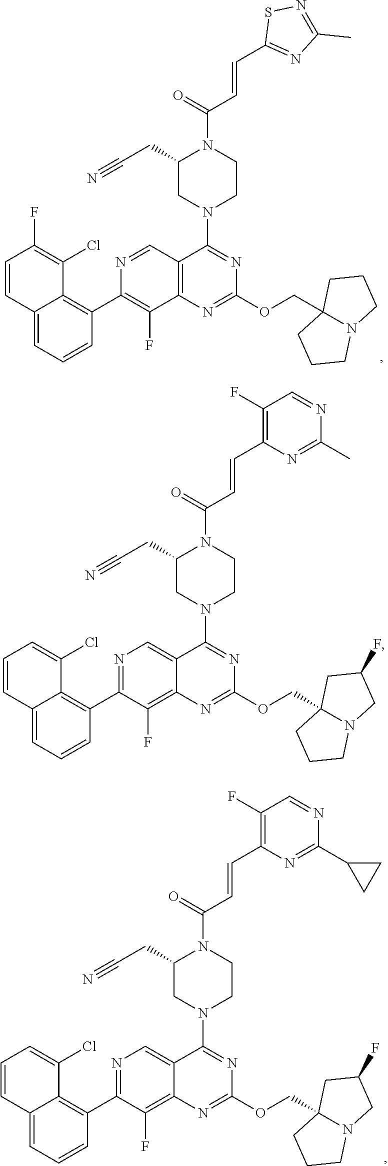

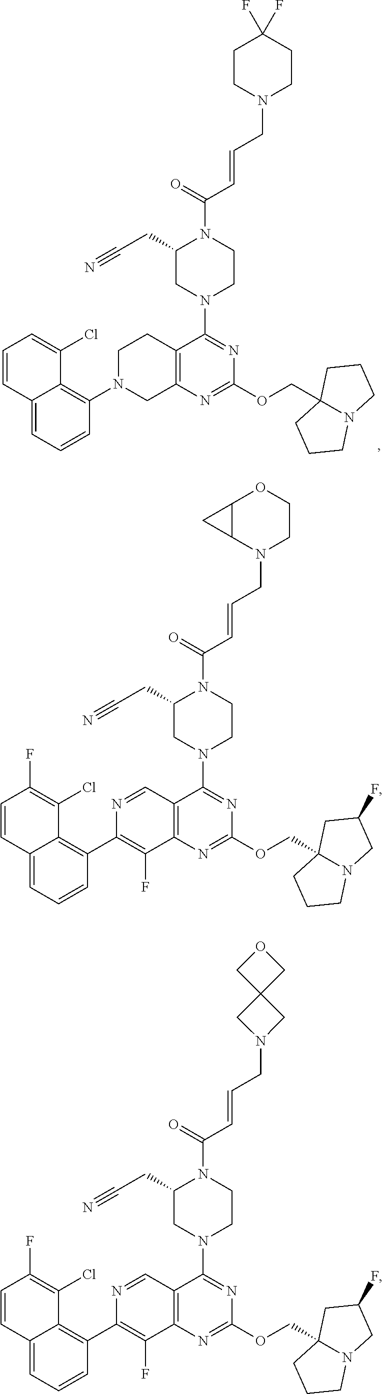 [SMILES: N#CC[C@H]1CN(c2nc(OCC34CCCN3CCC4)nc3c2CCN(c2cccc4cccc(Cl)c24)C3)CCN1C(=O)/C=C/CN1CCC(F)(F)CC1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCOC2CC21 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2(COC2)C1]

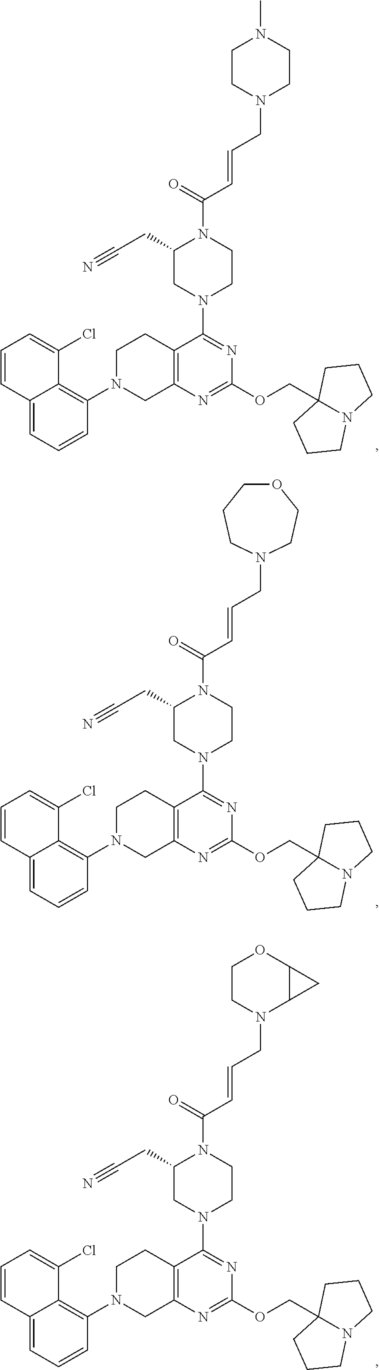

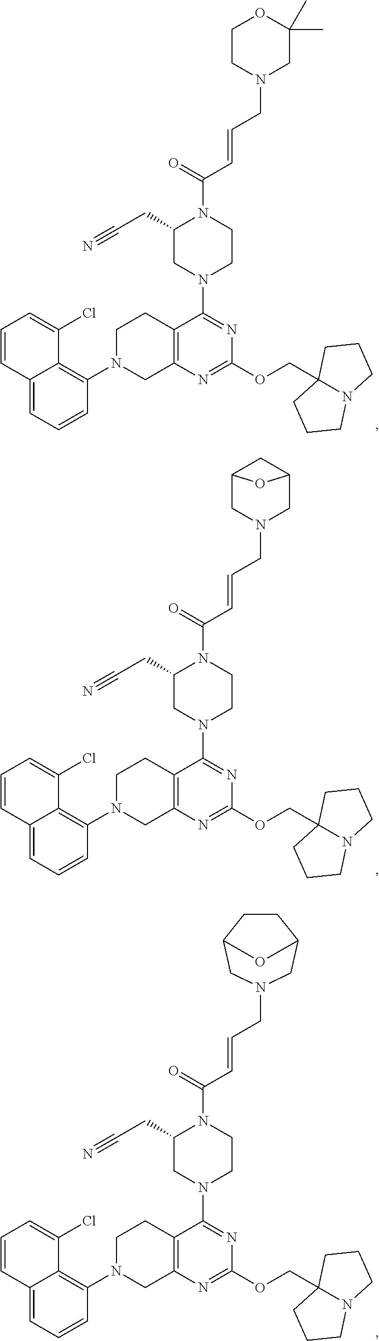

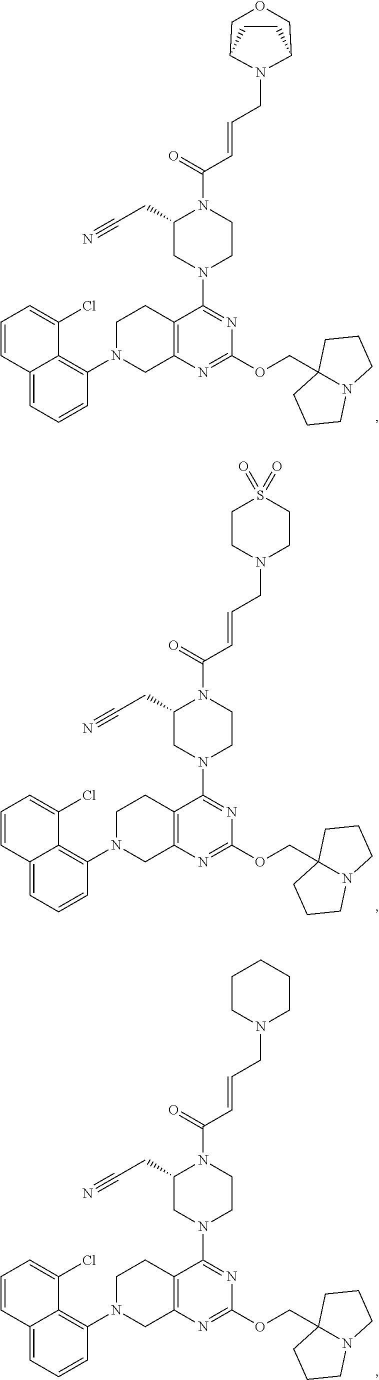 [SMILES: N#CC[C@H]1CN(c2nc(OCC34CCCN3CCC4)nc3c2CCN(c2cccc4cccc(Cl)c24)C3)CCN1C(=O)/C=C/CN1[C@@H]2CC[C@H]1COC2 ; N#CC[C@H]1CN(c2nc(OCC34CCCN3CCC4)nc3c2CCN(c2cccc4cccc(Cl)c24)C3)CCN1C(=O)/C=C/CN1CCS(=O)(=O)CC1 ; N#CC[C@H]1CN(c2nc(OCC34CCCN3CCC4)nc3c2CCN(c2cccc4cccc(Cl)c24)C3)CCN1C(=O)/C=C/CN1CCCCC1]

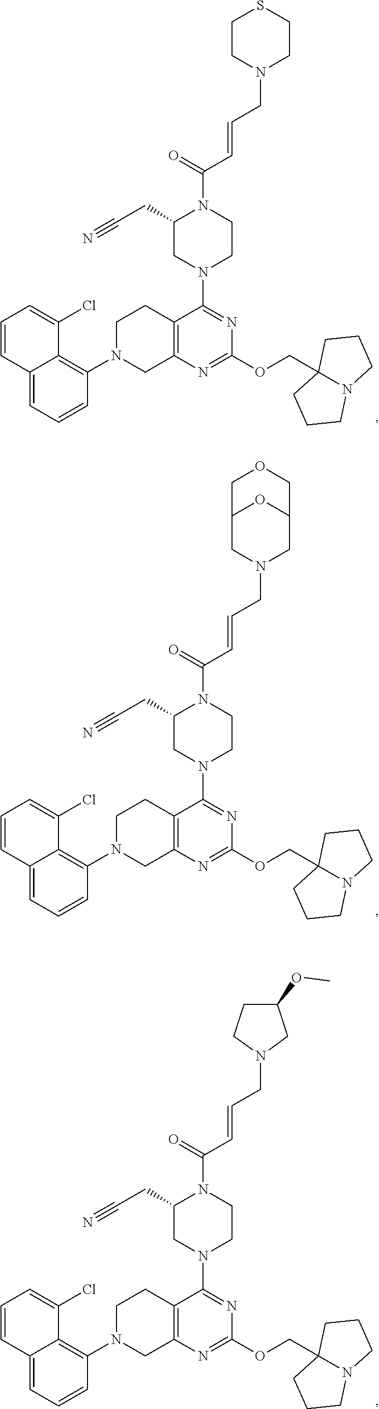 [SMILES: N#CC[C@H]1CN(c2nc(OCC34CCCN3CCC4)nc3c2CCN(c2cccc4cccc(Cl)c24)C3)CCN1C(=O)/C=C/CN1CCSCC1 ; N#CC[C@H]1CN(c2nc(OCC34CCCN3CCC4)nc3c2CCN(c2cccc4cccc(Cl)c24)C3)CCN1C(=O)/C=C/CN1CC2COCC(C1)O2 ; CO[C@@H]1CCN(C/C=C/C(=O)N2CCN(c3nc(OCC45CCCN4CCC5)nc4c3CCN(c3cccc5cccc(Cl)c35)C4)C[C@@H]2CC#N)C1]

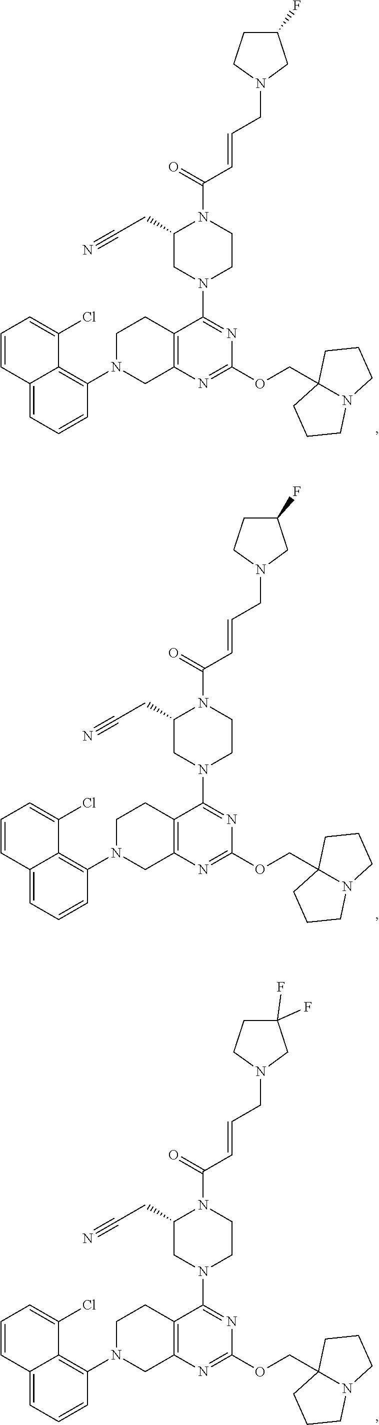 [SMILES: N#CC[C@H]1CN(c2nc(OCC34CCCN3CCC4)nc3c2CCN(c2cccc4cccc(Cl)c24)C3)CCN1C(=O)/C=C/CN1CC[C@H](F)C1 ; N#CC[C@H]1CN(c2nc(OCC34CCCN3CCC4)nc3c2CCN(c2cccc4cccc(Cl)c24)C3)CCN1C(=O)/C=C/CN1CC[C@@H](F)C1 ; N#CC[C@H]1CN(c2nc(OCC34CCCN3CCC4)nc3c2CCN(c2cccc4cccc(Cl)c24)C3)CCN1C(=O)/C=C/CN1CCC(F)(F)C1]

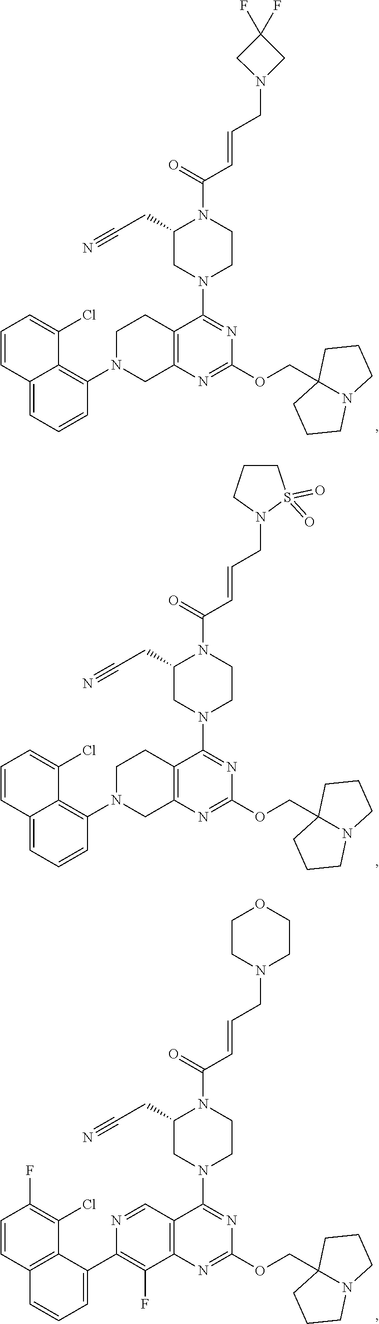

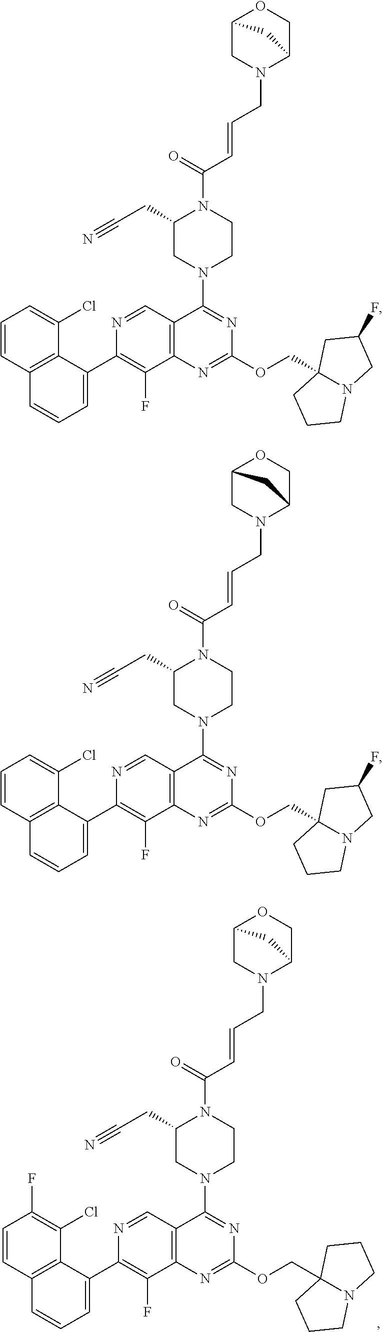 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@@H]2C[C@H]1CO2 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@H]2C[C@@H]1CO2 ; N#CC[C@H]1CN(c2nc(OCC34CCCN3CCC4)nc3c(F)c(-c4cccc5ccc(F)c(Cl)c45)ncc23)CCN1C(=O)/C=C/CN1C[C@@H]2C[C@H]1CO2]

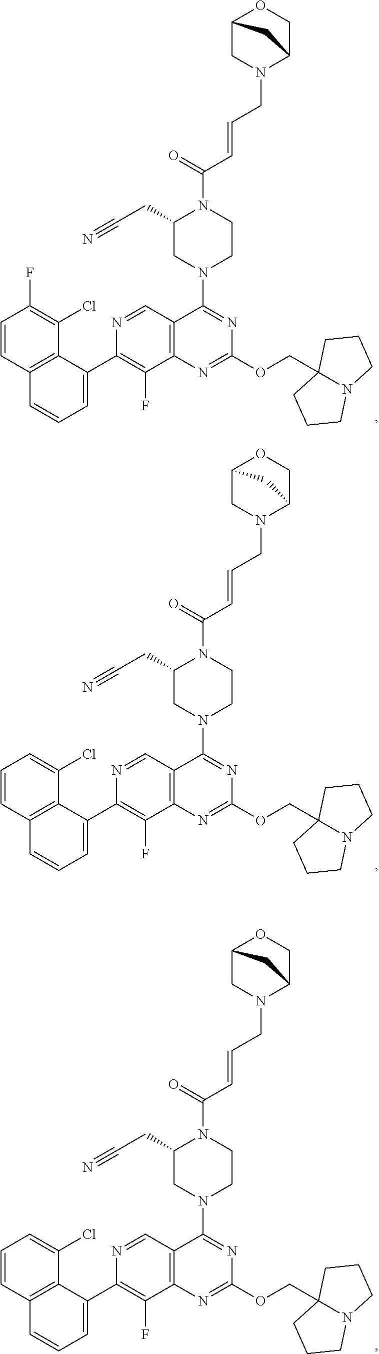 [SMILES: N#CC[C@H]1CN(c2nc(OCC34CCCN3CCC4)nc3c(F)c(-c4cccc5ccc(F)c(Cl)c45)ncc23)CCN1C(=O)/C=C/CN1C[C@H]2C[C@@H]1CO2 ; N#CC[C@H]1CN(c2nc(OCC34CCCN3CCC4)nc3c(F)c(-c4cccc5cccc(Cl)c45)ncc23)CCN1C(=O)/C=C/CN1C[C@@H]2C[C@H]1CO2 ; N#CC[C@H]1CN(c2nc(OCC34CCCN3CCC4)nc3c(F)c(-c4cccc5cccc(Cl)c45)ncc23)CCN1C(=O)/C=C/CN1C[C@H]2C[C@@H]1CO2]

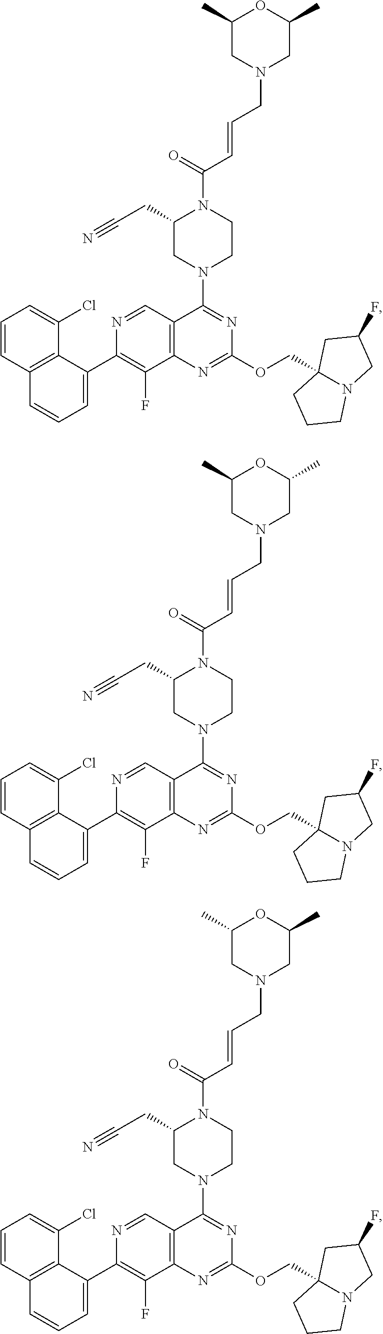

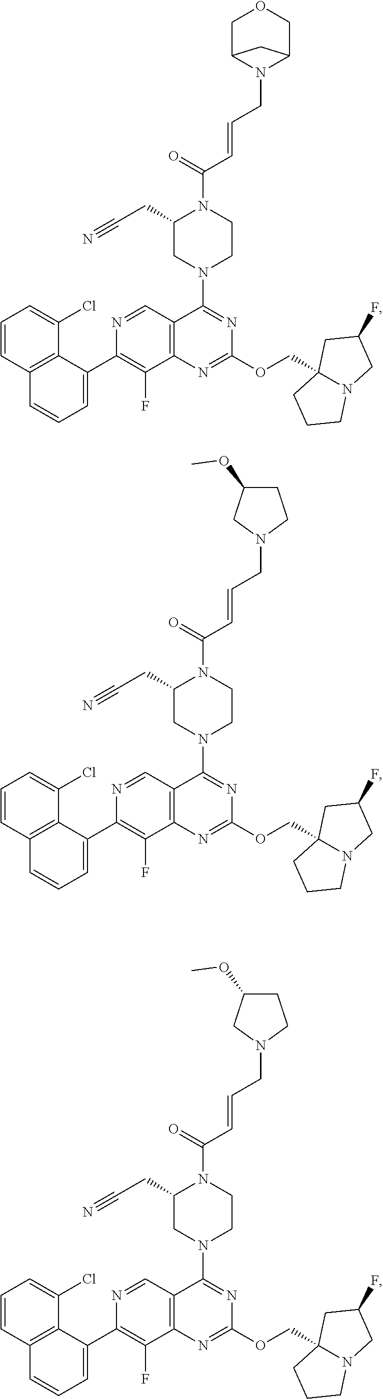 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C2COCC1C2 ; CO[C@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1 ; CO[C@@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1]

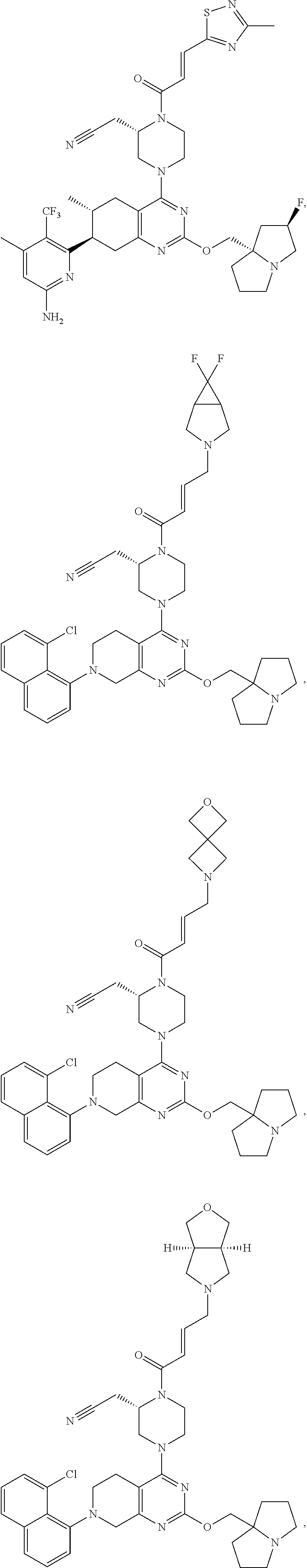 [SMILES: Cc1nsc(/C=C/C(=O)N2CCN(c3nc(OC[C@@]45CCCN4C[C@H](F)C5)nc4c3C[C@@H](C)[C@H](c3nc(N)cc(C)c3C(F)(F)F)C4)C[C@@H]2CC#N)n1 ; N#CC[C@H]1CN(c2nc(OCC34CCCN3CCC4)nc3c2CCN(c2cccc4cccc(Cl)c24)C3)CCN1C(=O)/C=C/CN1CC2C(C1)C2(F)F ; N#CC[C@H]1CN(c2nc(OCC34CCCN3CCC4)nc3c2CCN(c2cccc4cccc(Cl)c24)C3)CCN1C(=O)/C=C/CN1CC2(COC2)C1 ; N#CC[C@H]1CN(c2nc(OCC34CCCN3CCC4)nc3c2CCN(c2cccc4cccc(Cl)c24)C3)CCN1C(=O)/C=C/CN1C[C@H]2COC[C@H]2C1]

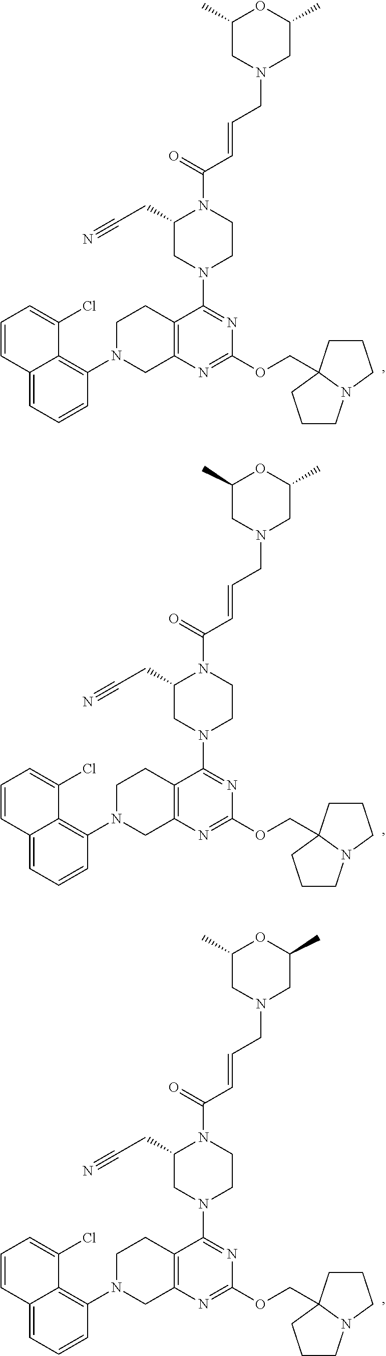 [SMILES: C[C@@H]1CN(C/C=C/C(=O)N2CCN(c3nc(OCC45CCCN4CCC5)nc4c3CCN(c3cccc5cccc(Cl)c35)C4)C[C@@H]2CC#N)C[C@H](C)O1 ; C[C@@H]1CN(C/C=C/C(=O)N2CCN(c3nc(OCC45CCCN4CCC5)nc4c3CCN(c3cccc5cccc(Cl)c35)C4)C[C@@H]2CC#N)C[C@@H](C)O1 ; C[C@H]1CN(C/C=C/C(=O)N2CCN(c3nc(OCC45CCCN4CCC5)nc4c3CCN(c3cccc5cccc(Cl)c35)C4)C[C@@H]2CC#N)C[C@H](C)O1]

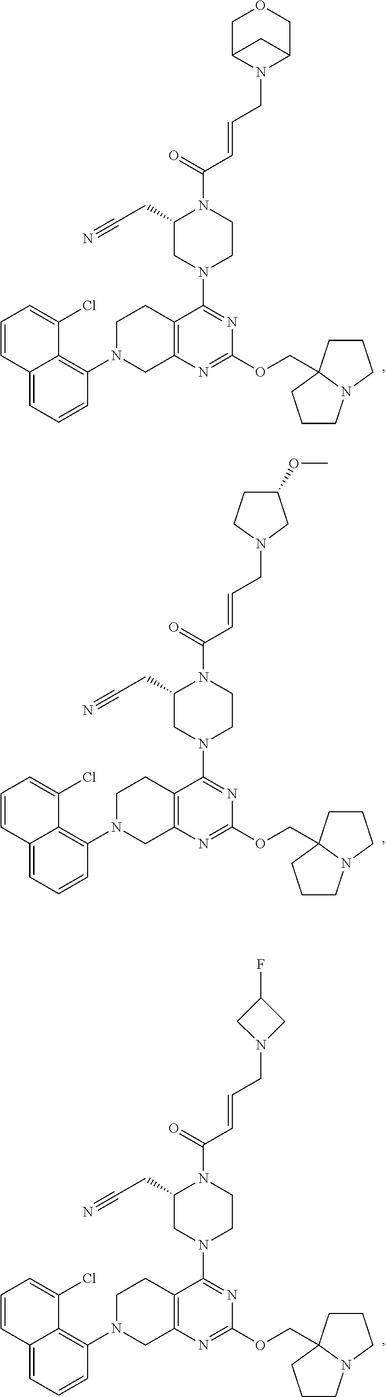

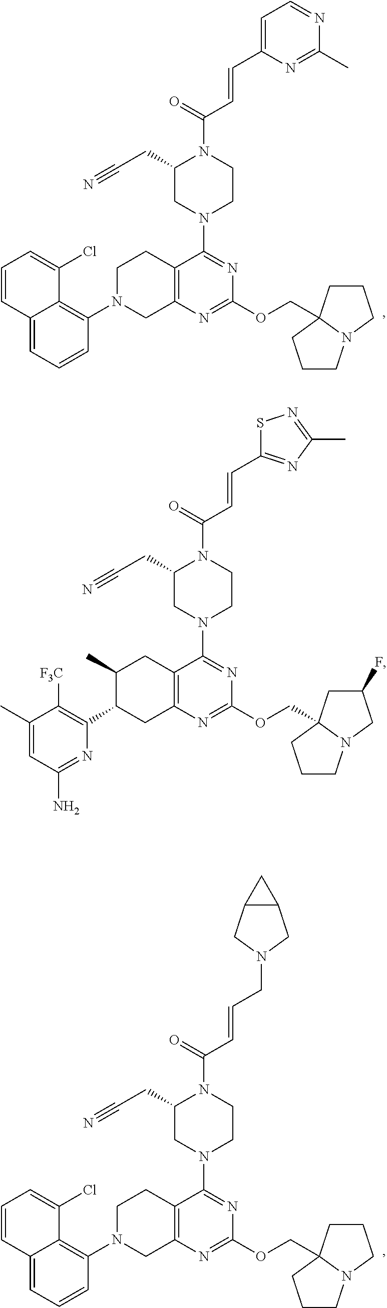

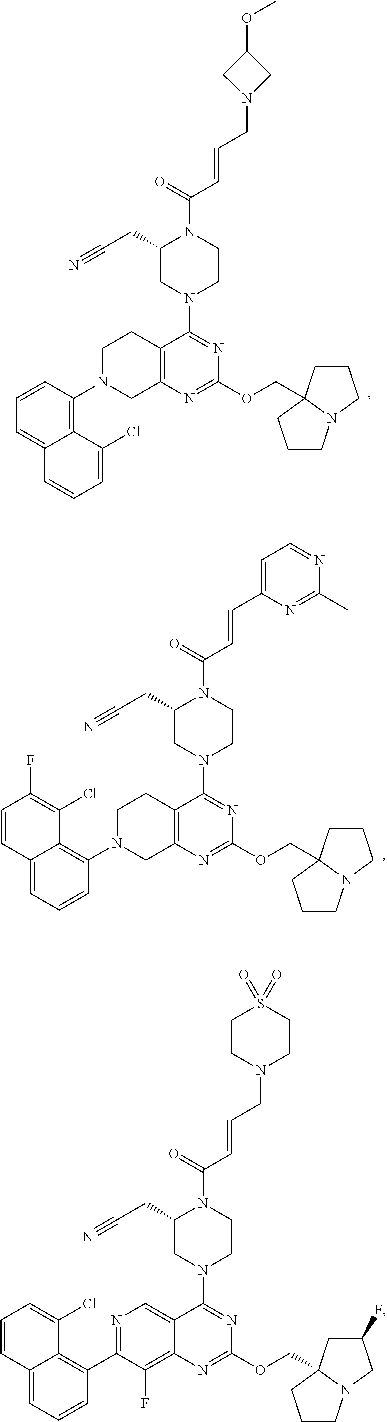 [SMILES: COC1CN(C/C=C/C(=O)N2CCN(c3nc(OCC45CCCN4CCC5)nc4c3CCN(c3cccc5cccc(Cl)c35)C4)C[C@@H]2CC#N)C1 ; Cc1nccc(/C=C/C(=O)N2CCN(c3nc(OCC45CCCN4CCC5)nc4c3CCN(c3cccc5ccc(F)c(Cl)c35)C4)C[C@@H]2CC#N)n1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCS(=O)(=O)CC1]

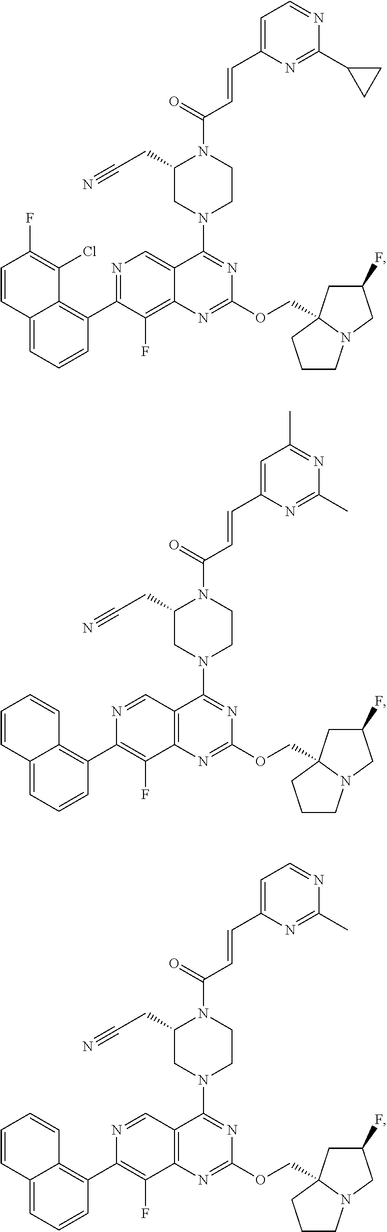

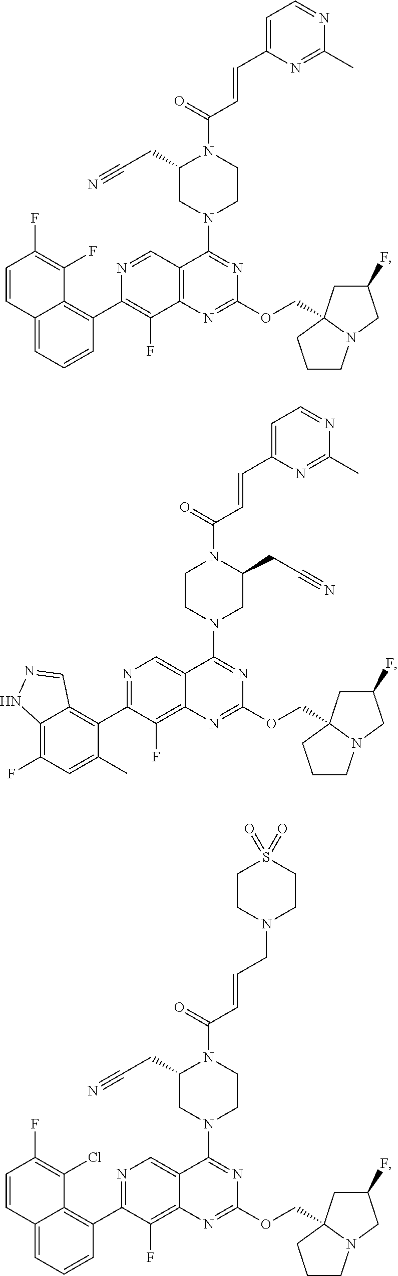

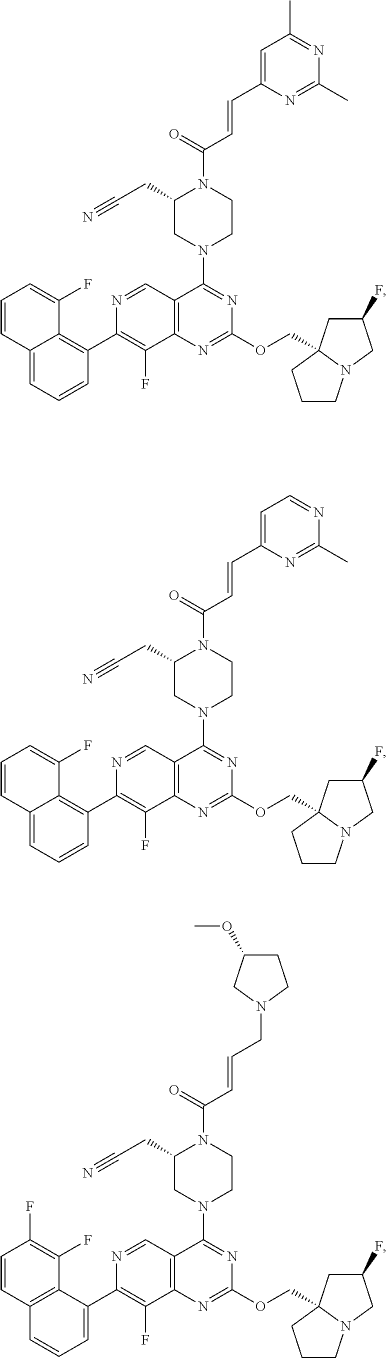 [SMILES: Cc1cc(/C=C/C(=O)N2CCN(c3nc(OC[C@@]45CCCN4C[C@H](F)C5)nc4c(F)c(-c5cccc6cccc(F)c56)ncc34)C[C@@H]2CC#N)nc(C)n1 ; Cc1nccc(/C=C/C(=O)N2CCN(c3nc(OC[C@@]45CCCN4C[C@H](F)C5)nc4c(F)c(-c5cccc6cccc(F)c56)ncc34)C[C@@H]2CC#N)n1 ; CO[C@@H]1CCN(C/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)C1]

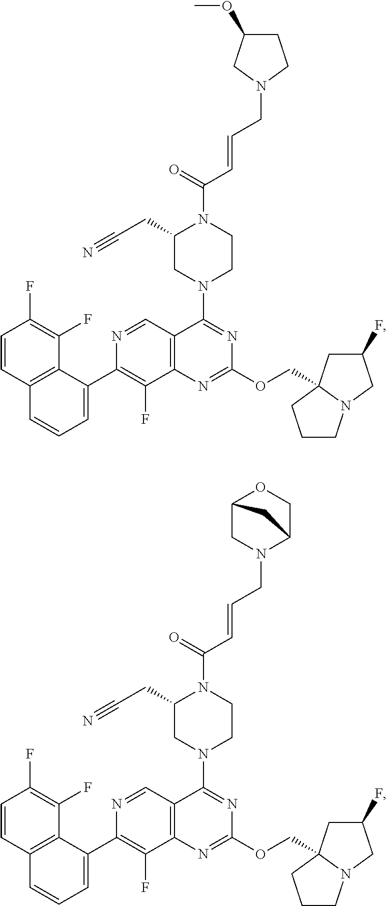 [SMILES: CO[C@H]1CCN(C/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)C1 ; N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1C[C@H]2C[C@@H]1CO2]

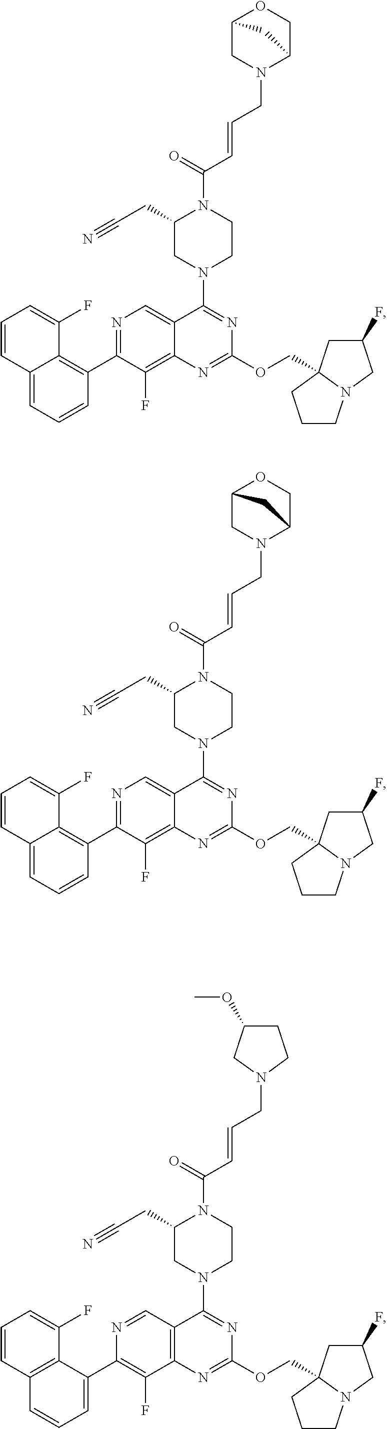 [SMILES: N#CC[C@H]1CN(c2nc(OC[C@@]34CCCN3C[C@H](F)C4)nc3c(F)c(-c4cccc5cccc(F)c45)ncc23)CCN1C(=O)/C=C/CN1C[C@@H]2C[C@H]1CO2 ; N#CC[C@H]1CN(c2nc(OC[C@@]34CCCN3C[C@H](F)C4)nc3c(F)c(-c4cccc5cccc(F)c45)ncc23)CCN1C(=O)/C=C/CN1C[C@H]2C[C@@H]1CO2 ; CO[C@@H]1CCN(C/C=C/C(=O)N2CCN(c3nc(OC[C@@]45CCCN4C[C@H](F)C5)nc4c(F)c(-c5cccc6cccc(F)c56)ncc34)C[C@@H]2CC#N)C1]

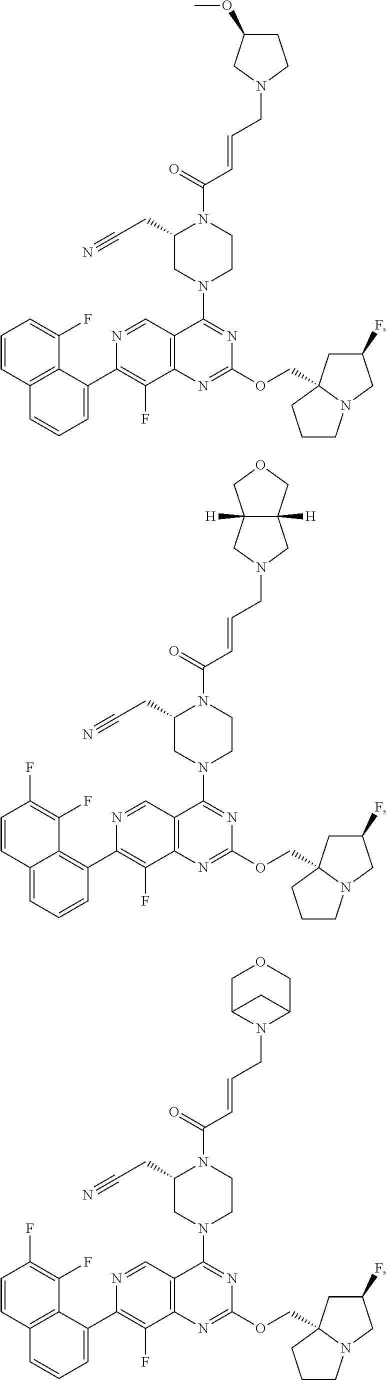 [SMILES: CO[C@H]1CCN(C/C=C/C(=O)N2CCN(c3nc(OC[C@@]45CCCN4C[C@H](F)C5)nc4c(F)c(-c5cccc6cccc(F)c56)ncc34)C[C@@H]2CC#N)C1 ; N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1C[C@H]2COC[C@H]2C1 ; N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1C2COCC1C2]

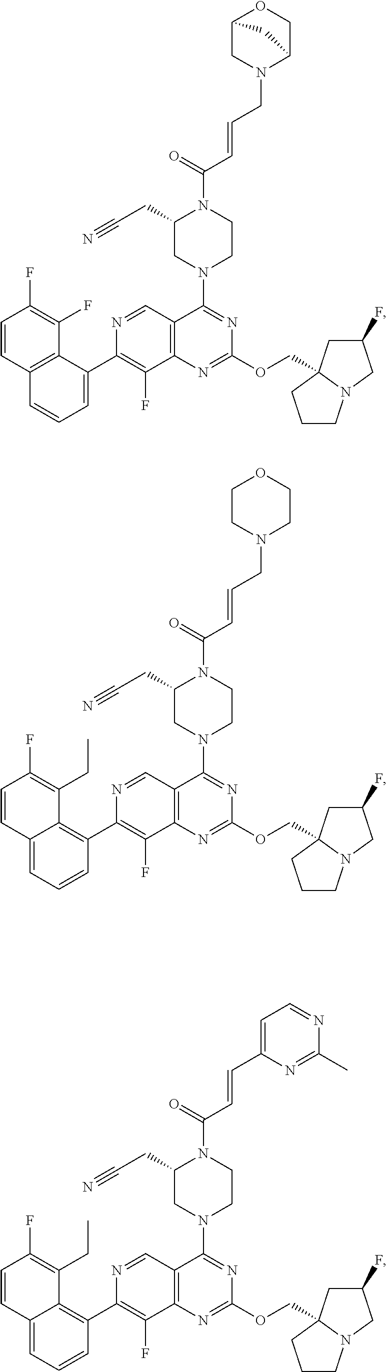 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1C[C@@H]2C[C@H]1CO2 ; CCc1c(F)ccc2cccc(-c3ncc4c(N5CCN(C(=O)/C=C/CN6CCOCC6)[C@@H](CC#N)C5)nc(OC[C@@]56CCCN5C[C@H](F)C6)nc4c3F)c12 ; CCc1c(F)ccc2cccc(-c3ncc4c(N5CCN(C(=O)/C=C/c6ccnc(C)n6)[C@@H](CC#N)C5)nc(OC[C@@]56CCCN5C[C@H](F)C6)nc4c3F)c12]

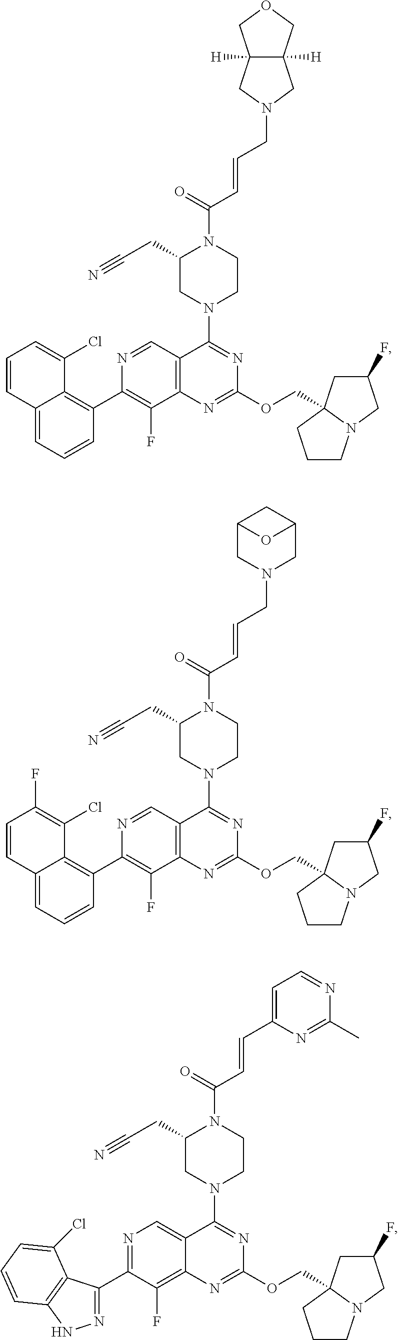 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@H]2COC[C@H]2C1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2CC(C1)O2 ; Cc1nccc(/C=C/C(=O)N2CCN(c3nc(OC[C@@]45CCCN4C[C@H](F)C5)nc4c(F)c(-c5n[nH]c6cccc(Cl)c56)ncc34)C[C@@H]2CC#N)n1]

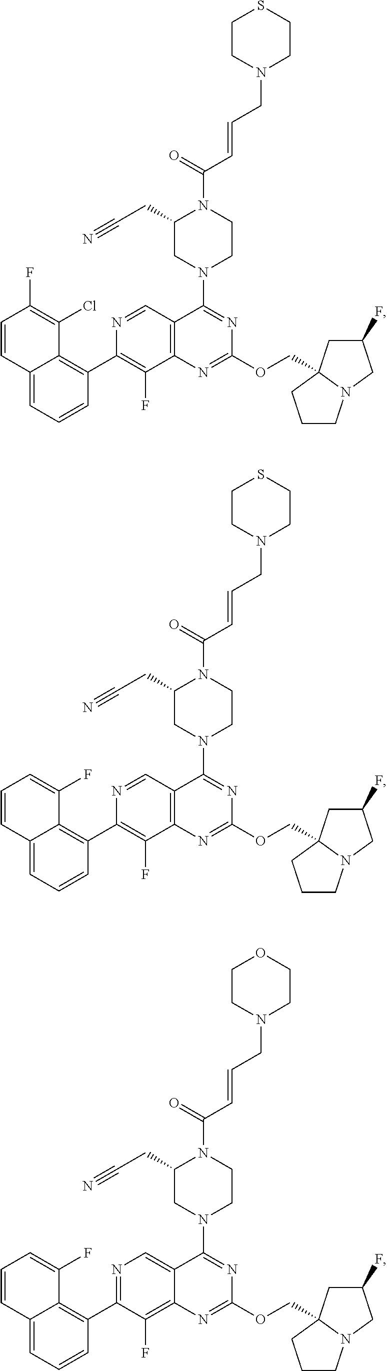 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCSCC1 ; N#CC[C@H]1CN(c2nc(OC[C@@]34CCCN3C[C@H](F)C4)nc3c(F)c(-c4cccc5cccc(F)c45)ncc23)CCN1C(=O)/C=C/CN1CCSCC1 ; N#CC[C@H]1CN(c2nc(OC[C@@]34CCCN3C[C@H](F)C4)nc3c(F)c(-c4cccc5cccc(F)c45)ncc23)CCN1C(=O)/C=C/CN1CCOCC1]

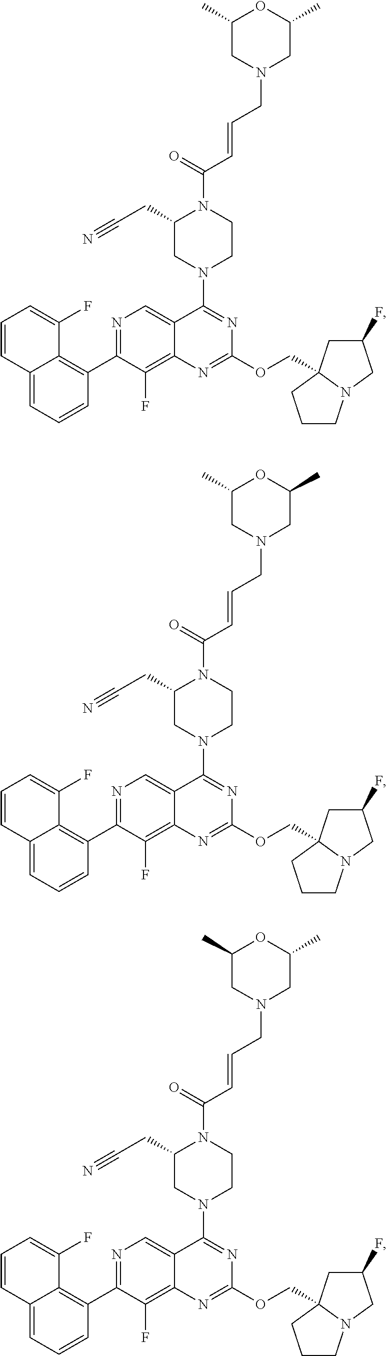 [SMILES: C[C@@H]1CN(C/C=C/C(=O)N2CCN(c3nc(OC[C@@]45CCCN4C[C@H](F)C5)nc4c(F)c(-c5cccc6cccc(F)c56)ncc34)C[C@@H]2CC#N)C[C@H](C)O1 ; C[C@H]1CN(C/C=C/C(=O)N2CCN(c3nc(OC[C@@]45CCCN4C[C@H](F)C5)nc4c(F)c(-c5cccc6cccc(F)c56)ncc34)C[C@@H]2CC#N)C[C@H](C)O1 ; C[C@@H]1CN(C/C=C/C(=O)N2CCN(c3nc(OC[C@@]45CCCN4C[C@H](F)C5)nc4c(F)c(-c5cccc6cccc(F)c56)ncc34)C[C@@H]2CC#N)C[C@@H](C)O1]

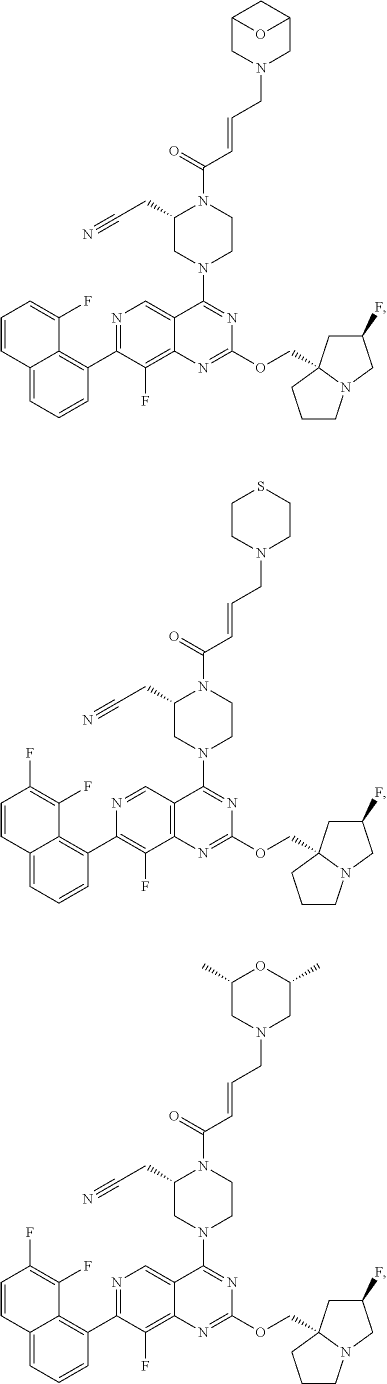 [SMILES: N#CC[C@H]1CN(c2nc(OC[C@@]34CCCN3C[C@H](F)C4)nc3c(F)c(-c4cccc5cccc(F)c45)ncc23)CCN1C(=O)/C=C/CN1CC2CC(C1)O2 ; N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCSCC1 ; C[C@@H]1CN(C/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)C[C@H](C)O1]

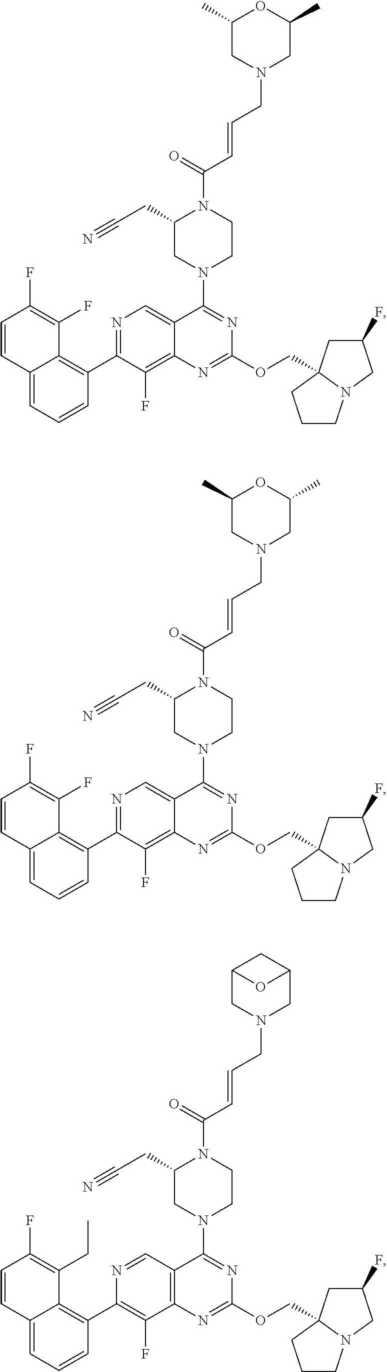 [SMILES: C[C@H]1CN(C/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)C[C@H](C)O1 ; C[C@@H]1CN(C/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)C[C@@H](C)O1 ; CCc1c(F)ccc2cccc(-c3ncc4c(N5CCN(C(=O)/C=C/CN6CC7CC(C6)O7)[C@@H](CC#N)C5)nc(OC[C@@]56CCCN5C[C@H](F)C6)nc4c3F)c12]

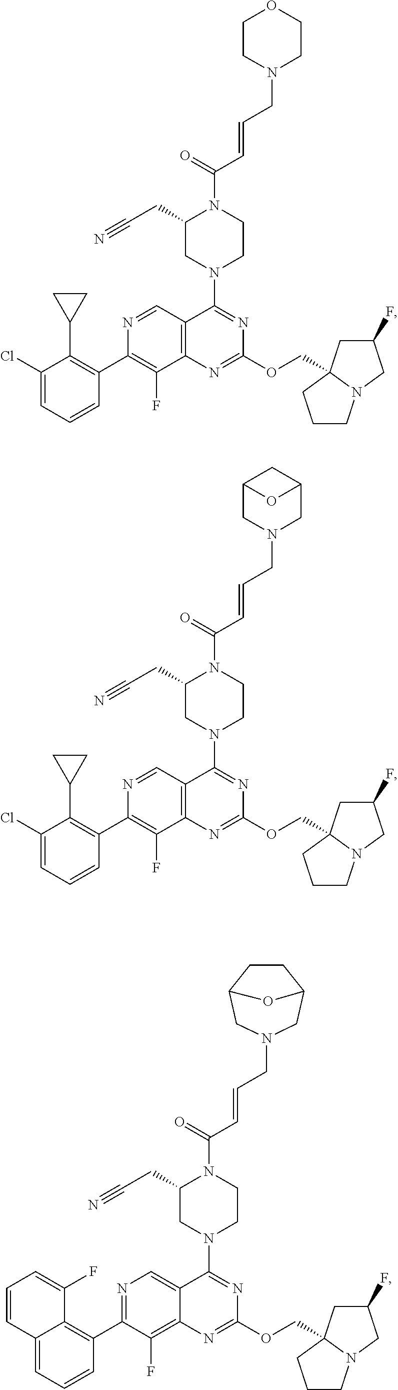 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CCOCC1 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CC2CC(C1)O2 ; N#CC[C@H]1CN(c2nc(OC[C@@]34CCCN3C[C@H](F)C4)nc3c(F)c(-c4cccc5cccc(F)c45)ncc23)CCN1C(=O)/C=C/CN1CC2CCC(C1)O2]

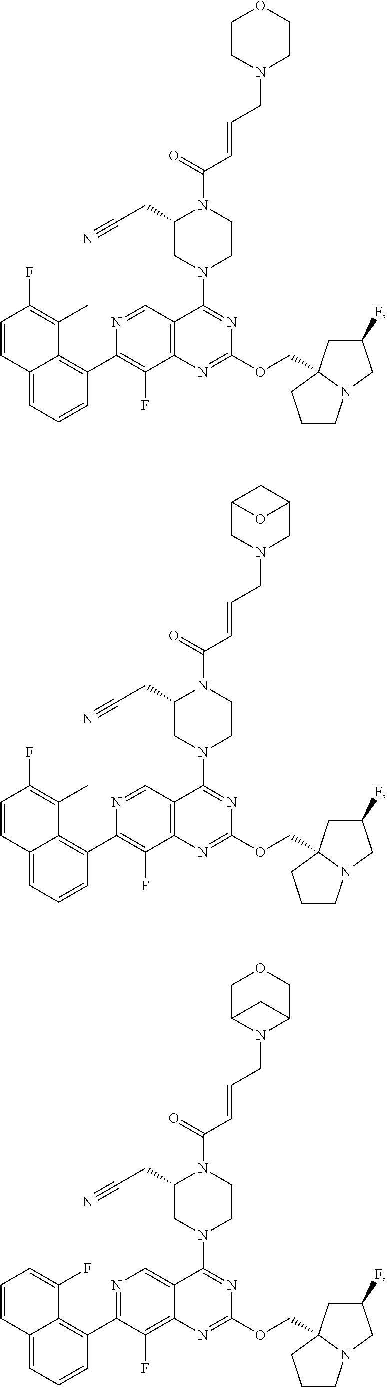 [SMILES: Cc1c(F)ccc2cccc(-c3ncc4c(N5CCN(C(=O)/C=C/CN6CCOCC6)[C@@H](CC#N)C5)nc(OC[C@@]56CCCN5C[C@H](F)C6)nc4c3F)c12 ; Cc1c(F)ccc2cccc(-c3ncc4c(N5CCN(C(=O)/C=C/CN6CC7CC(C6)O7)[C@@H](CC#N)C5)nc(OC[C@@]56CCCN5C[C@H](F)C6)nc4c3F)c12 ; N#CC[C@H]1CN(c2nc(OC[C@@]34CCCN3C[C@H](F)C4)nc3c(F)c(-c4cccc5cccc(F)c45)ncc23)CCN1C(=O)/C=C/CN1C2COCC1C2]

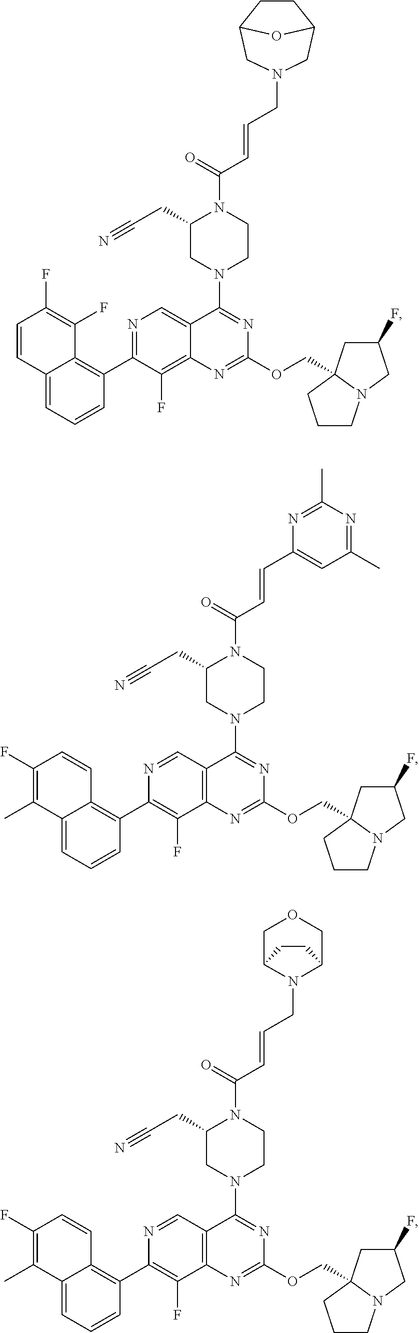

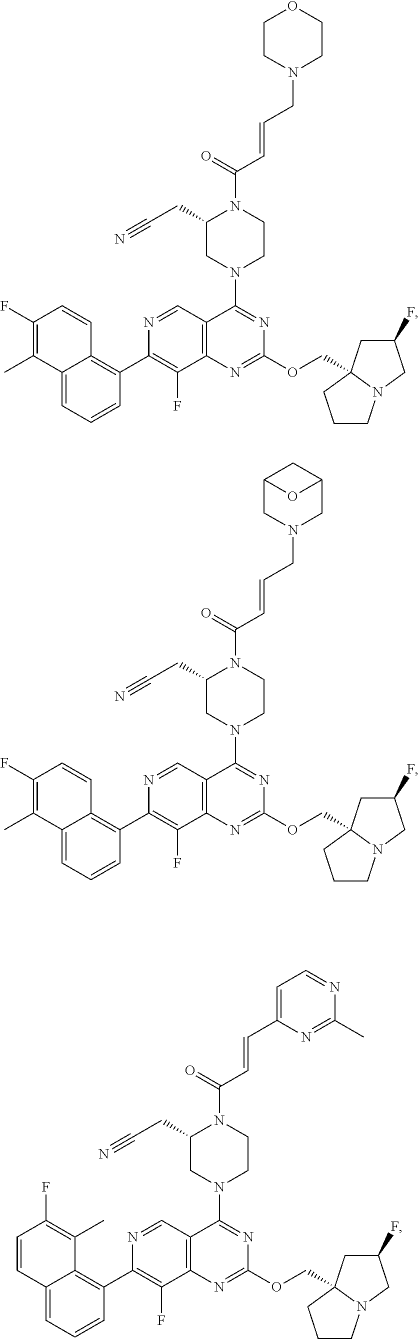 [SMILES: Cc1c(F)ccc2c(-c3ncc4c(N5CCN(C(=O)/C=C/CN6CCOCC6)[C@@H](CC#N)C5)nc(OC[C@@]56CCCN5C[C@H](F)C6)nc4c3F)cccc12 ; Cc1c(F)ccc2c(-c3ncc4c(N5CCN(C(=O)/C=C/CN6CC7CC(C6)O7)[C@@H](CC#N)C5)nc(OC[C@@]56CCCN5C[C@H](F)C6)nc4c3F)cccc12 ; Cc1nccc(/C=C/C(=O)N2CCN(c3nc(OC[C@@]45CCCN4C[C@H](F)C5)nc4c(F)c(-c5cccc6ccc(F)c(C)c56)ncc34)C[C@@H]2CC#N)n1]

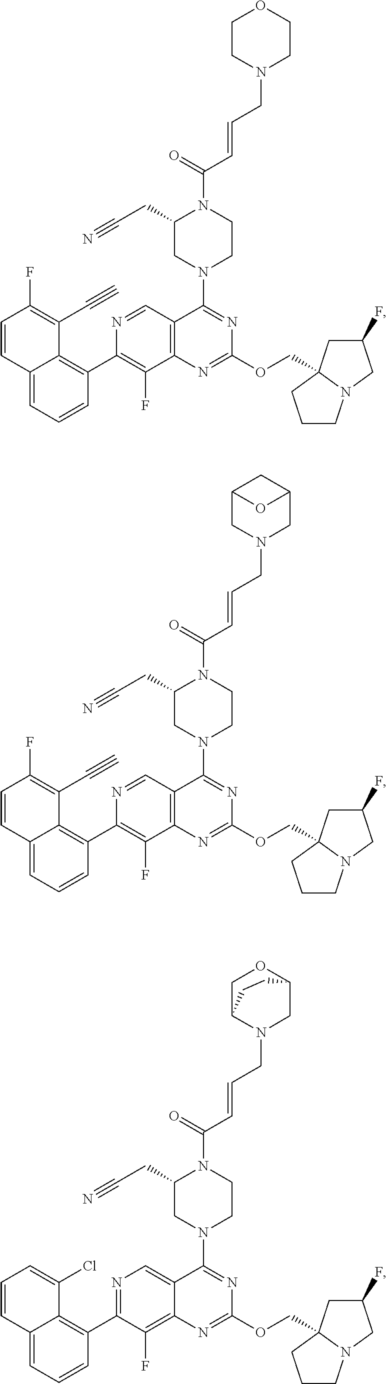 [SMILES: C#Cc1c(F)ccc2cccc(-c3ncc4c(N5CCN(C(=O)/C=C/CN6CCOCC6)[C@@H](CC#N)C5)nc(OC[C@@]56CCCN5C[C@H](F)C6)nc4c3F)c12 ; C#Cc1c(F)ccc2cccc(-c3ncc4c(N5CCN(C(=O)/C=C/CN6CC7CC(C6)O7)[C@@H](CC#N)C5)nc(OC[C@@]56CCCN5C[C@H](F)C6)nc4c3F)c12 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@H]2CC[C@@H]1CO2]

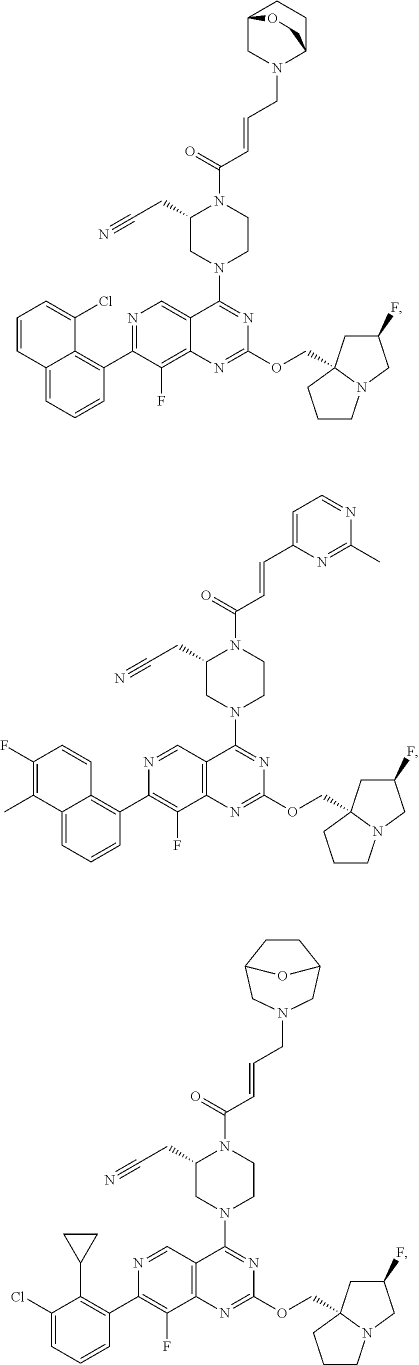 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@@H]2CC[C@H]1CO2 ; Cc1nccc(/C=C/C(=O)N2CCN(c3nc(OC[C@@]45CCCN4C[C@H](F)C5)nc4c(F)c(-c5cccc6c(C)c(F)ccc56)ncc34)C[C@@H]2CC#N)n1 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CC2CCC(C1)O2]

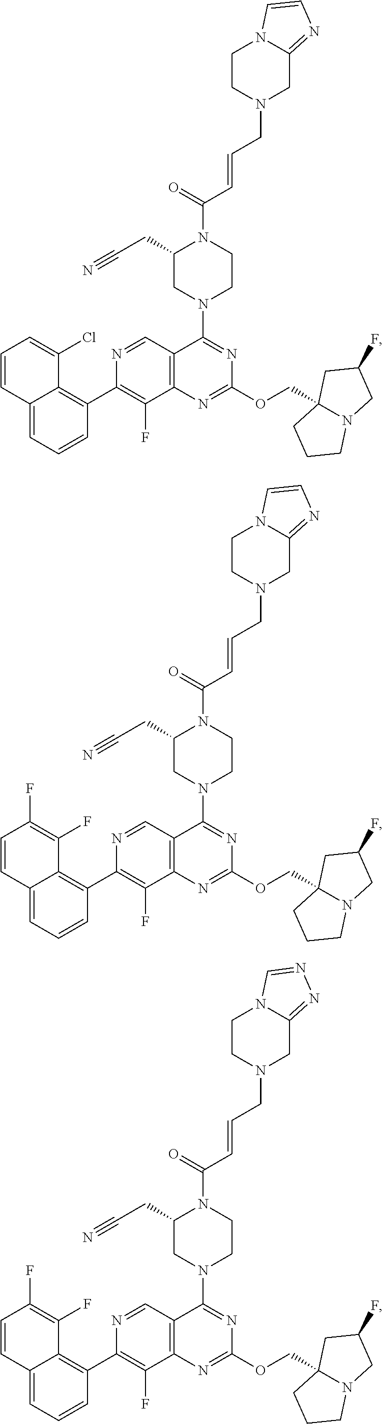

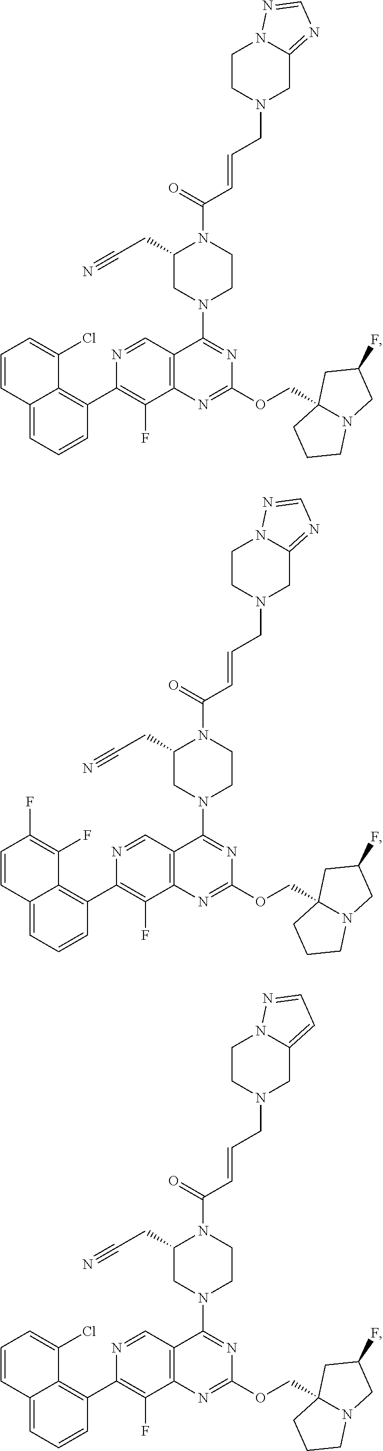

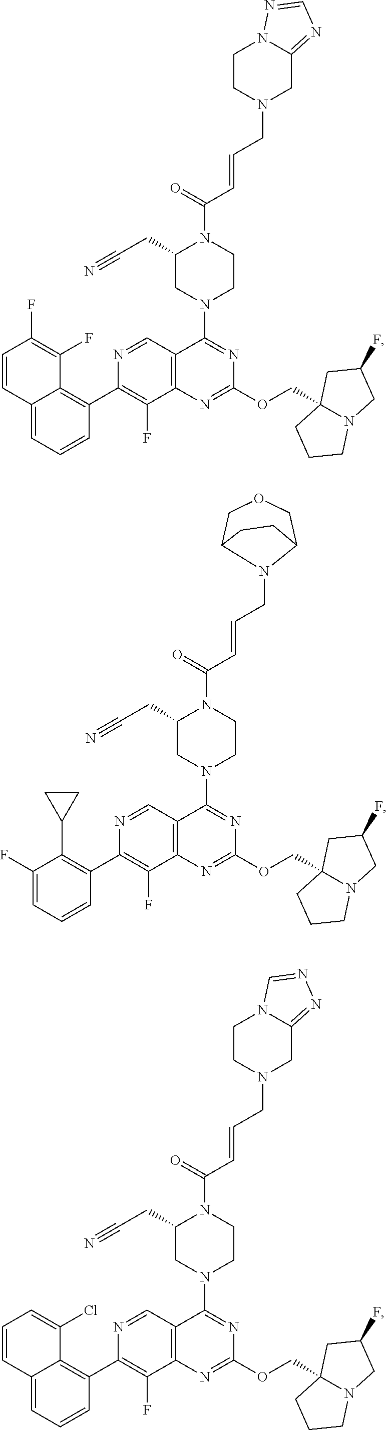 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCn2ncnc2C1 ; N#CC[C@H]1CN(c2nc(OC[C@@]34CCCN3C[C@H](F)C4)nc3c(F)c(-c4cccc(F)c4C4CC4)ncc23)CCN1C(=O)/C=C/CN1C2CCC1COC2 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCn2cnnc2C1]

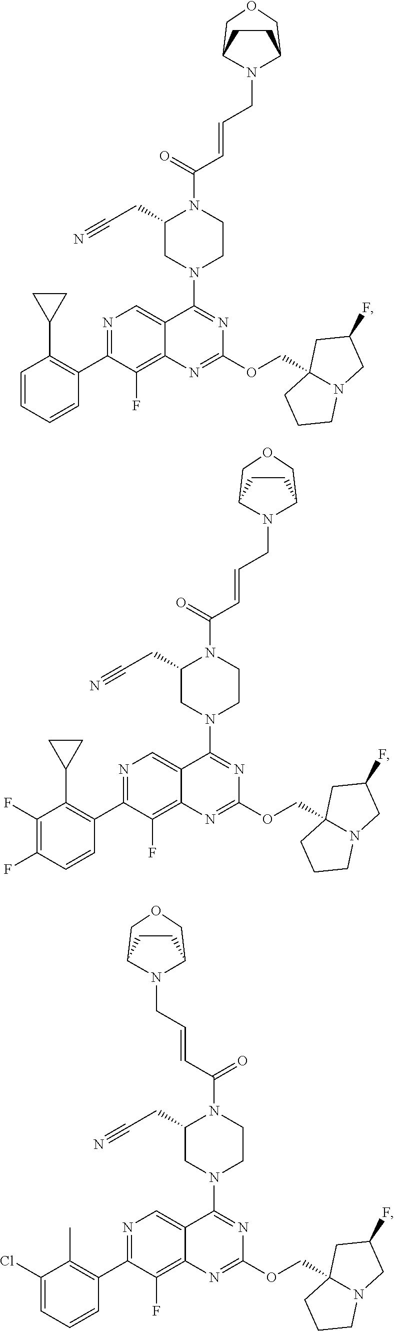

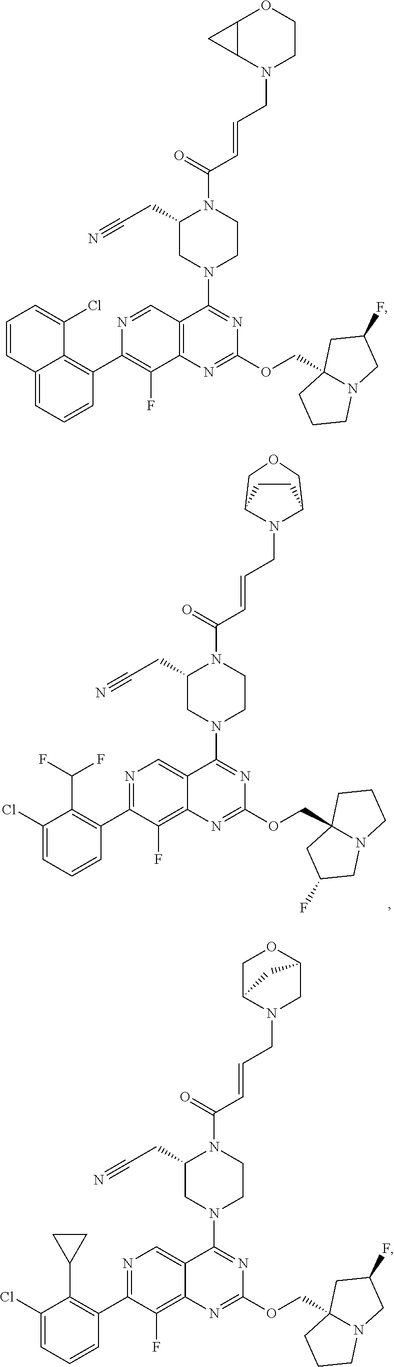 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCOC2CC21 ; N#CC[C@H]1CN(c2nc(OC[C@@]34CCCN3C[C@H](F)C4)nc3c(F)c(-c4cccc(Cl)c4C(F)F)ncc23)CCN1C(=O)/C=C/CN1[C@@H]2CC[C@H]1COC2 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1C[C@H]2C[C@@H]1CO2]

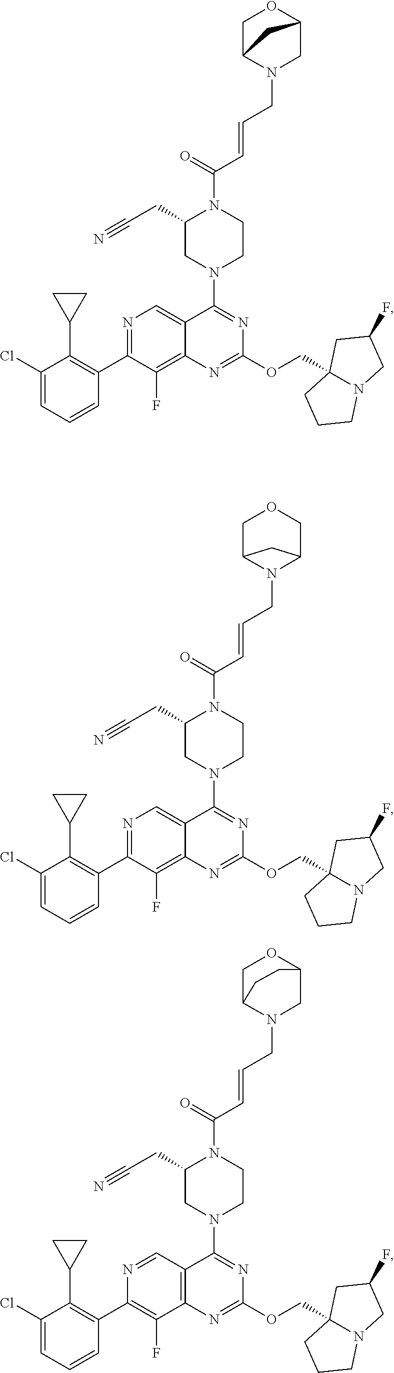 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1C[C@@H]2C[C@H]1CO2 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1C2COCC1C2 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CC2CCC1CO2]

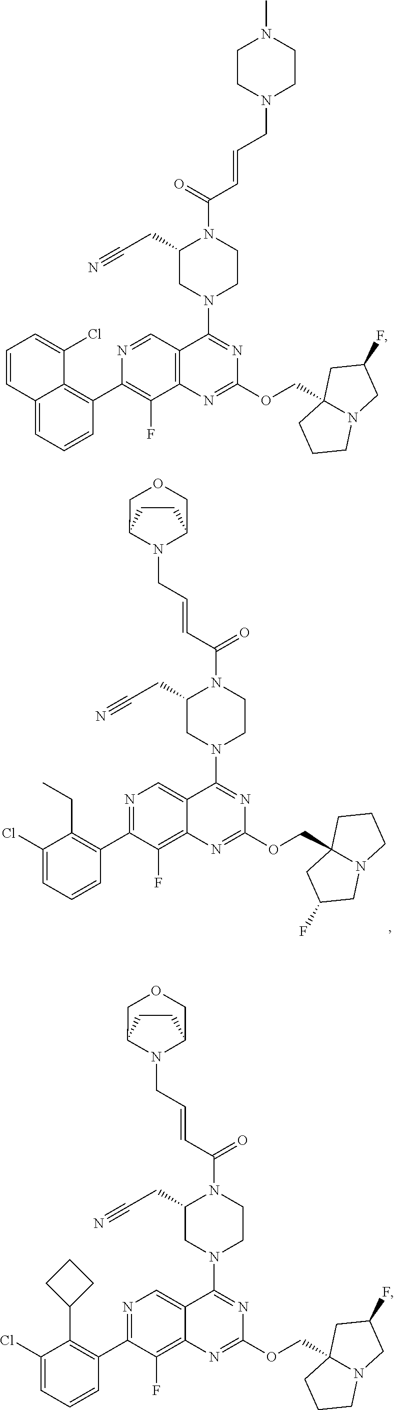 [SMILES: CN1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)CC1 ; CCc1c(Cl)cccc1-c1ncc2c(N3CCN(C(=O)/C=C/CN4[C@@H]5CC[C@H]4COC5)[C@@H](CC#N)C3)nc(OC[C@@]34CCCN3C[C@H](F)C4)nc2c1F ; N#CC[C@H]1CN(c2nc(OC[C@@]34CCCN3C[C@H](F)C4)nc3c(F)c(-c4cccc(Cl)c4C4CCC4)ncc23)CCN1C(=O)/C=C/CN1[C@@H]2CC[C@H]1COC2]

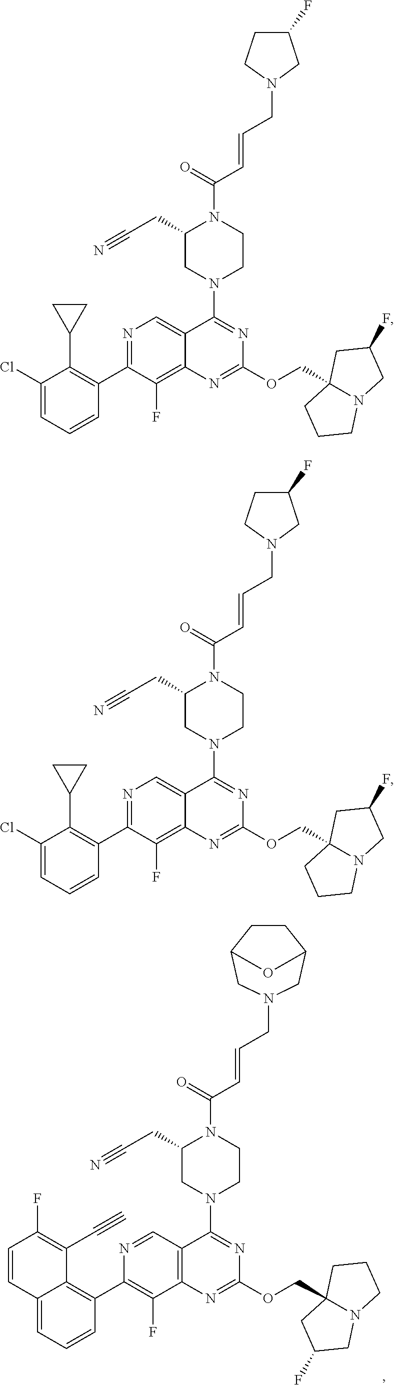

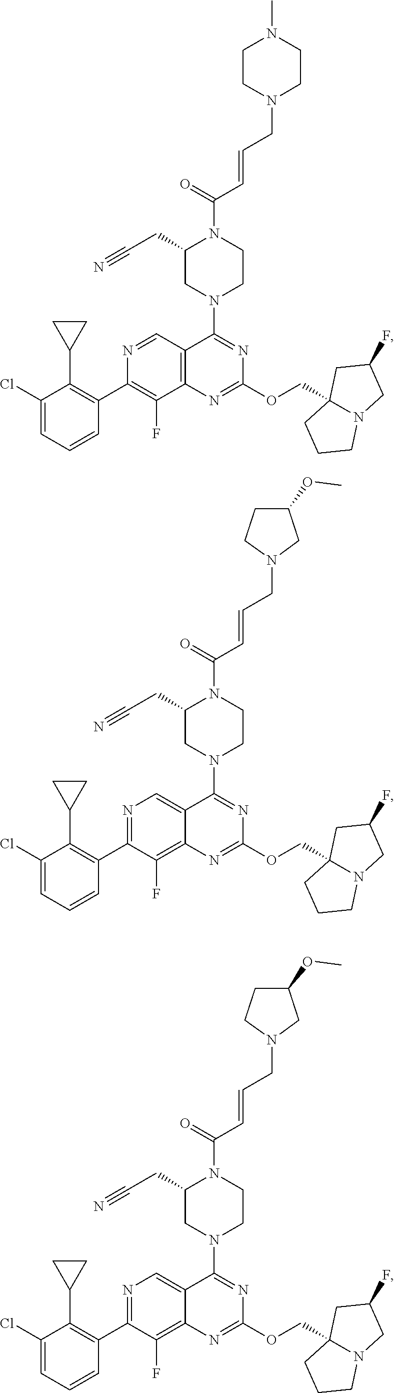

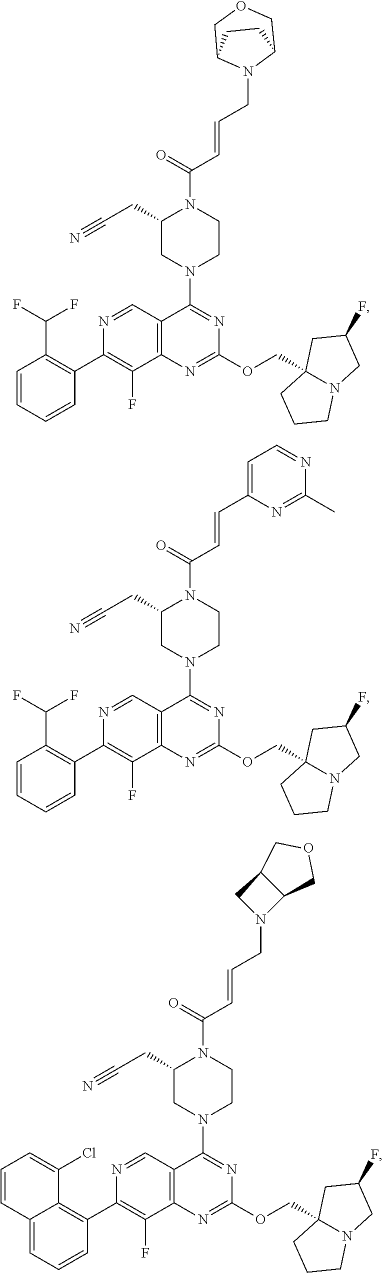

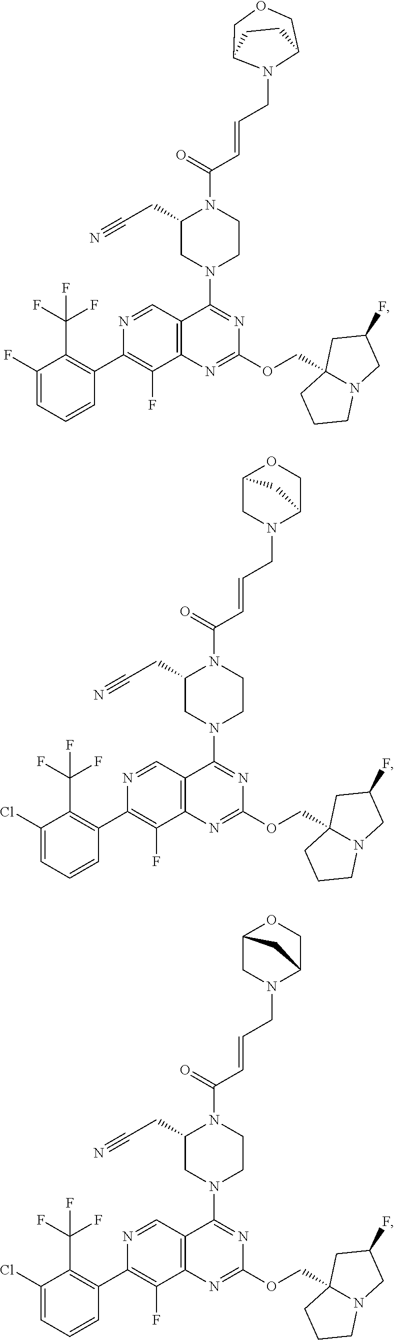 [SMILES: N#CC[C@H]1CN(c2nc(OC[C@@]34CCCN3C[C@H](F)C4)nc3c(F)c(-c4cccc(F)c4C(F)(F)F)ncc23)CCN1C(=O)/C=C/CN1[C@@H]2CC[C@H]1COC2 ; N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1C[C@@H]2C[C@H]1CO2 ; N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1C[C@H]2C[C@@H]1CO2]

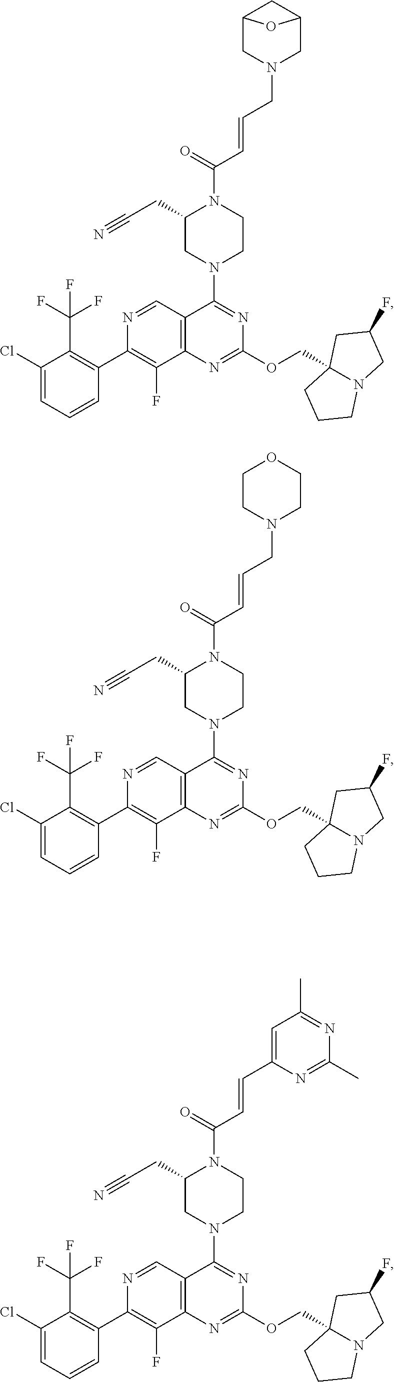 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CC2CC(C1)O2 ; N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CCOCC1 ; Cc1cc(/C=C/C(=O)N2CCN(c3nc(OC[C@@]45CCCN4C[C@H](F)C5)nc4c(F)c(-c5cccc(Cl)c5C(F)(F)F)ncc34)C[C@@H]2CC#N)nc(C)n1]

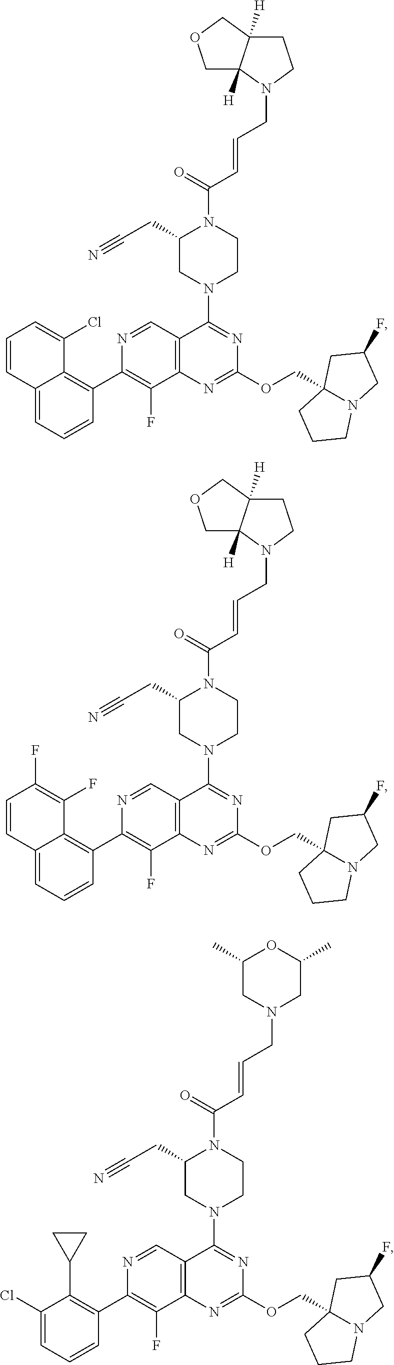

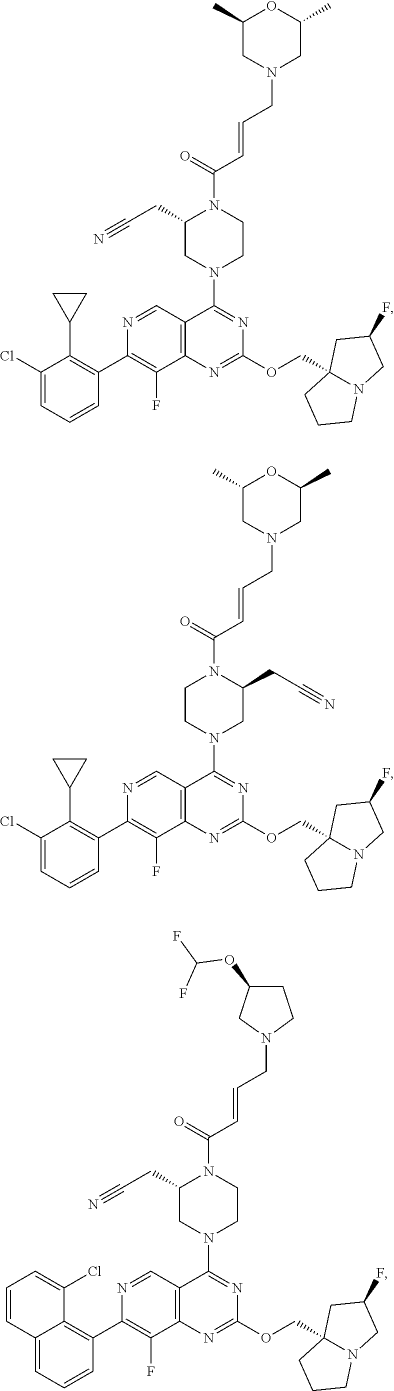 [SMILES: C[C@@H]1CN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)C[C@@H](C)O1 ; C[C@H]1CN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)C[C@H](C)O1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@H](OC(F)F)C1]

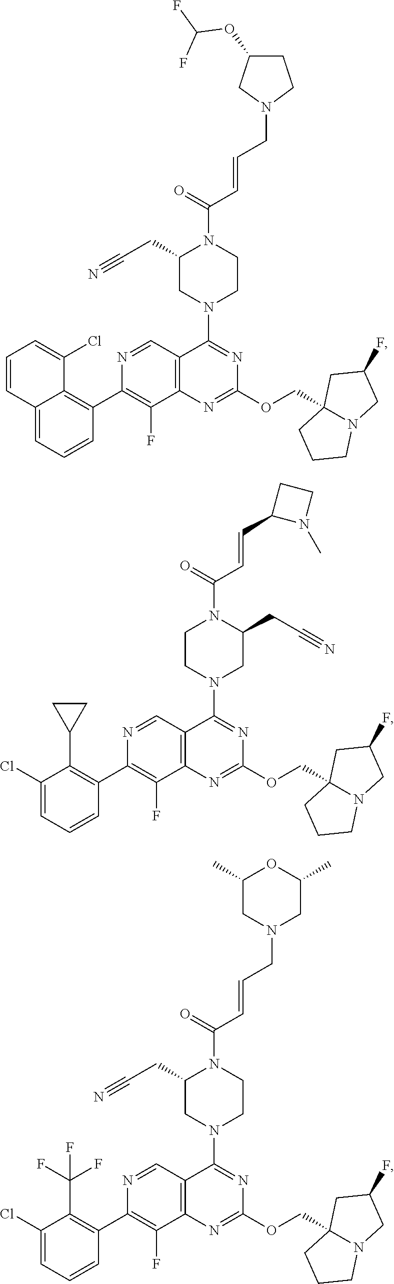

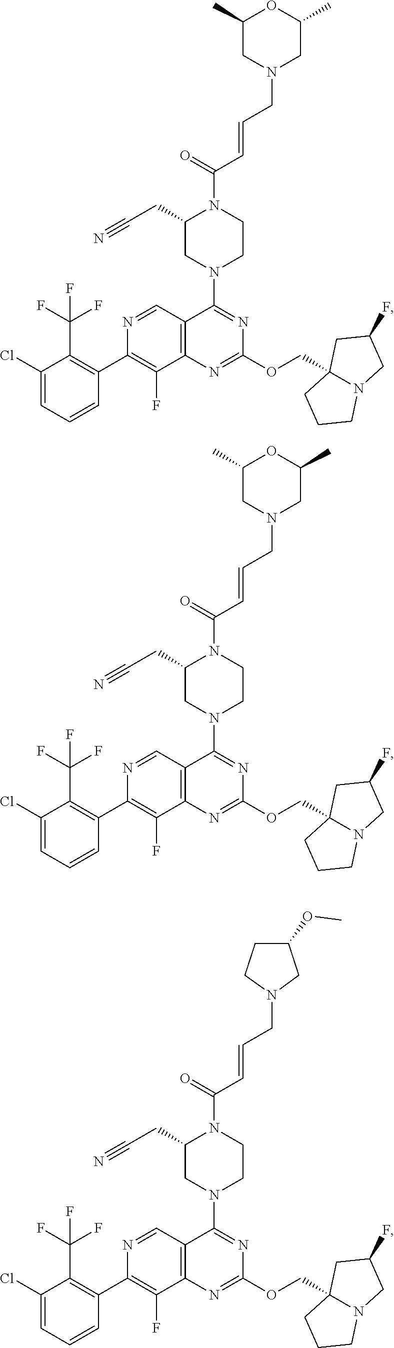

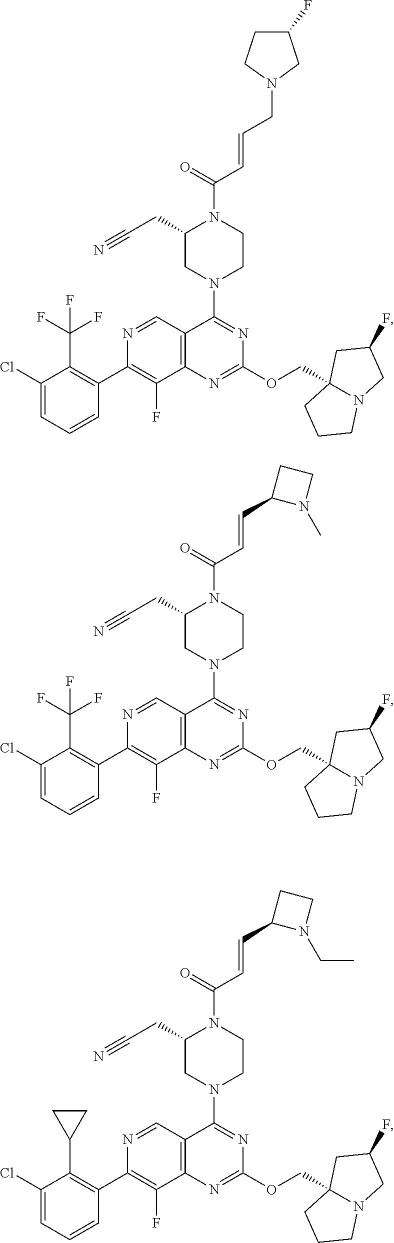

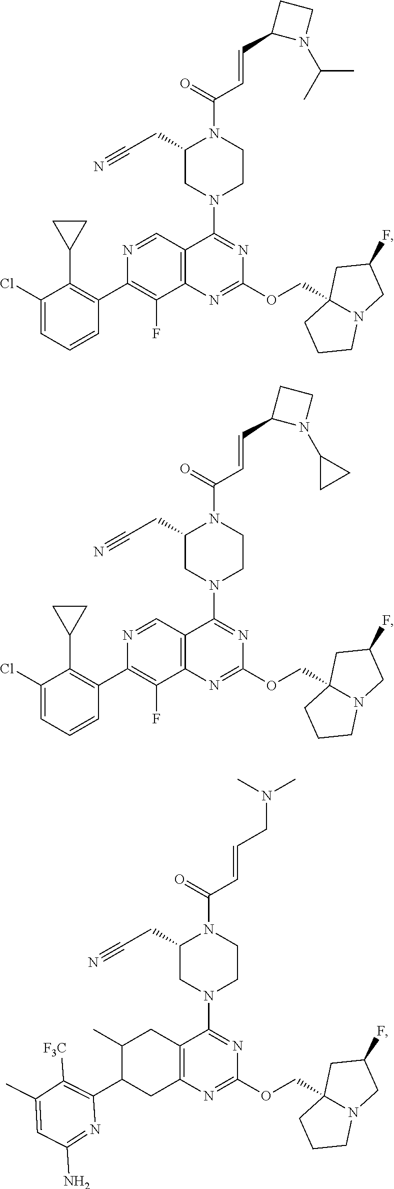

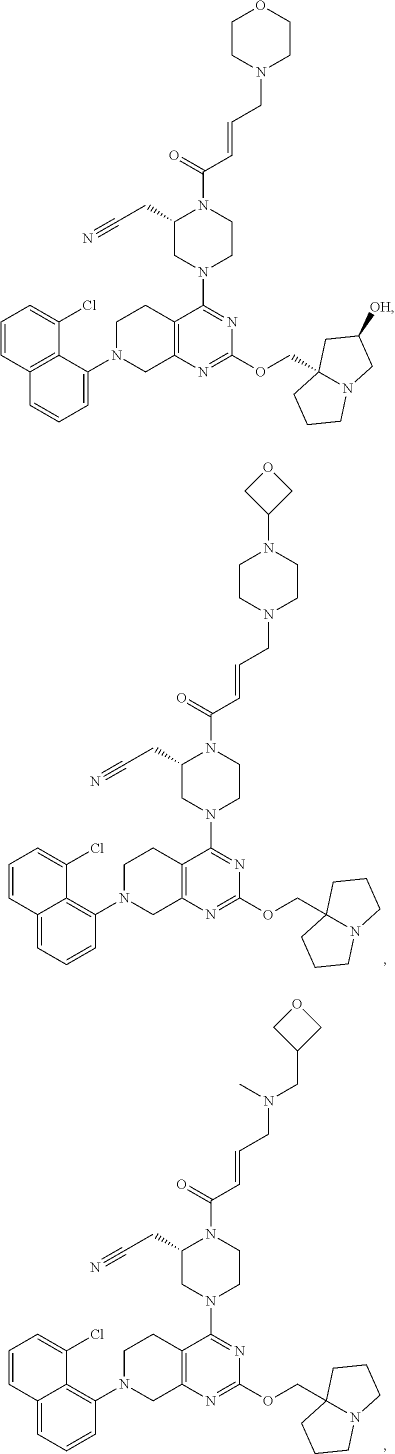

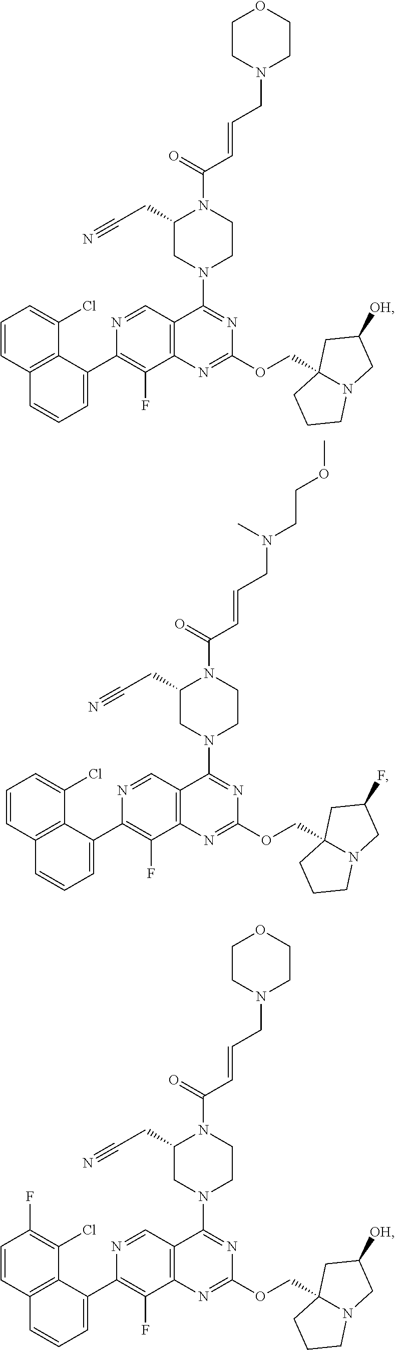

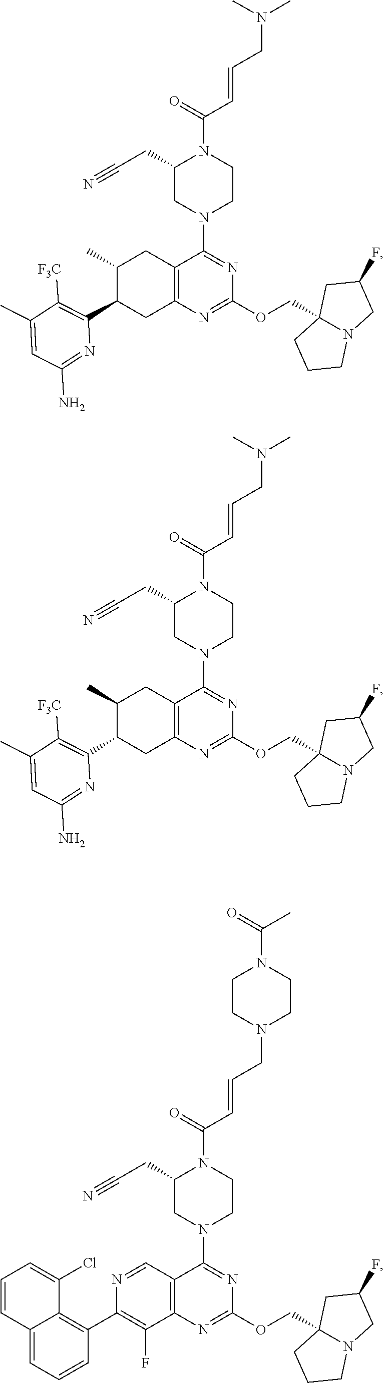 [SMILES: Cc1cc(N)nc([C@@H]2Cc3nc(OC[C@@]45CCCN4C[C@H](F)C5)nc(N4CCN(C(=O)/C=C/CN(C)C)[C@@H](CC#N)C4)c3C[C@H]2C)c1C(F)(F)F ; Cc1cc(N)nc([C@H]2Cc3nc(OC[C@@]45CCCN4C[C@H](F)C5)nc(N4CCN(C(=O)/C=C/CN(C)C)[C@@H](CC#N)C4)c3C[C@@H]2C)c1C(F)(F)F ; CC(=O)N1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)CC1]

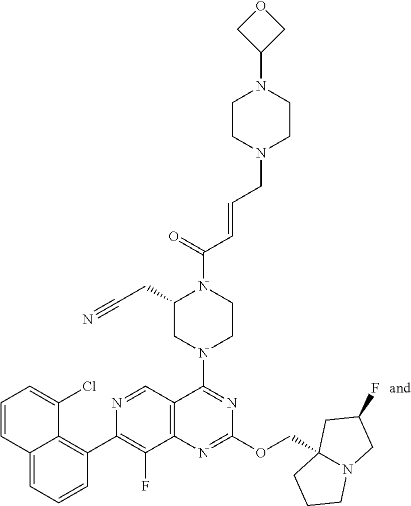 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCN(C2COC2)CC1]

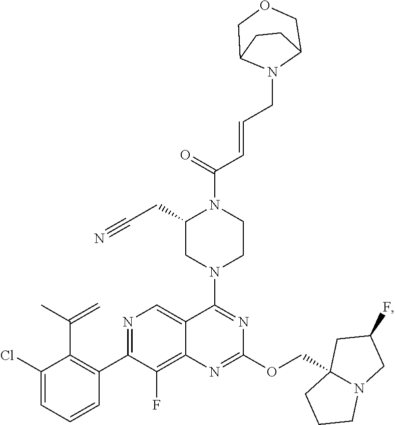 [SMILES: C=C(C)c1c(Cl)cccc1-c1ncc2c(N3CCN(C(=O)/C=C/CN4C5CCC4COC5)[C@@H](CC#N)C3)nc(OC[C@@]34CCCN3C[C@H](F)C4)nc2c1F]

or a salt thereof; and/or an isotopologue thereof.

In another aspect provided is a pharmaceutical formulation comprising a compound as described herein, including but not limited to a compound described in the preceding paragraphs, and a pharmaceutically acceptable carrier, wherein when the compound is a salt, the salt is a pharmaceutically acceptable salt.

In another aspect provided is a method of treating or suppressing cancer comprising: administering a therapeutically effective amount of a compound as described herein, including but not limited to a compound described in the preceding paragraphs, or a pharmaceutical formulation, including but not limited to the pharmaceutical formulation described in the preceding paragraphs, to a subject in need thereof, wherein when the compound is a salt, the salt is a pharmaceutically acceptable salt.

In another aspect is provided is the use of a compound as described herein, including but not limited to any of the foregoing embodiments, as a medicament.

In another aspect provided is a compound as described herein, including but not limited to any of the foregoing embodiments or a pharmaceutical formulation as described herein for use as a medicament.

In an aspect, provided is a compound as described herein, including but not limited to any of the foregoing embodiments or a pharmaceutical formulation as described in any of the embodiments described herein for use in treating or suppressing cancer.

In an aspect, provided is a compound as described herein, including but not limited to any of the foregoing embodiments or a pharmaceutical formulation as described in any of the embodiments described herein for use in the manufacturing of a medicament for treating or suppressing cancer, wherein when the compound is a salt, the salt is a pharmaceutically acceptable salt.

In an aspect, provided is a use of a compound as described herein, including but not limited to any of the foregoing embodiments or a pharmaceutical formulation as described in any of the embodiments described herein in the manufacturing of a medicament for treating or suppressing cancer, wherein when the compound is a salt, the salt is a pharmaceutically acceptable salt.

In an aspect, provided is a use of a compound as described herein, including but not limited to any of the foregoing embodiments or a pharmaceutical formulation as described in any of the embodiments described herein for treating or suppressing cancer, wherein when the compound is a salt, the salt is a pharmaceutically acceptable salt.

In some embodiments, the cancer is selected from the group consisting of: lung, colorectal, pancreatic, bile duct, thyroid, gall bladder, uterine, mesothelioma, cervical, and bladder cancers. In some embodiments, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.

It is to be understood that the description of compounds, compositions, formulations, and methods of treatment described herein include “comprising”, “consisting of”, and “consisting essentially of” embodiments. In some embodiments, for all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as ‘consisting essentially of’ a component, the composition may additionally contain any amount of pharmaceutically acceptable carriers, vehicles, or diluents and other such components which do not substantially affect the condition being treated.

Additional embodiments, features, and advantages of the present disclosure will be apparent from the following detailed description and through practice of the present disclosure.

### DETAILED DESCRIPTION

Provided herein are compounds useful in treating cancer, and methods of using such compounds for treating cancer. In some embodiments, the compounds are useful in treating cancers characterized by KRAS G12C. In some embodiments, the compounds advantageously inhibit both the inactive GDP- and activated GTP-bound forms of KRAS G12C. In some embodiments, the compounds advantageously have improved inhibition of the GTP-bound form of KRAS G12C.

The abbreviations used herein have their conventional meaning within the chemical and biological arts, unless otherwise specified.

It is to be understood that descriptions of compound structures, including possible substitutions, are limited to those which are chemically possible.

Unless otherwise indicated, the absolute stereochemistry of all chiral atoms is as depicted. Compounds with an (or) designation in the “Stereochemistry” column of Table 1 are single enantiomers wherein the absolute stereochemistry was arbitrarily assigned (e.g., based on chiral SFC elution as described in the Examples section). Compounds with an (and) designation in the stereochemistry column of Table 1 are mixtures of enantiomers wherein the relative stereochemistry is as shown. Compounds that have a stereogenic center where the configuration is not indicated in the structure as depicted and that have no designation in the Stereochemistry column of Table 1 are mixtures of enantiomers at that center. Compounds that have no designation in the Stereochemistry column of Table 1 or that are marked with (abs) are single enantiomers wherein the absolute stereochemistry is as indicated. For example, compound 2 is a single enantiomer with the stereochemistry as indicated.

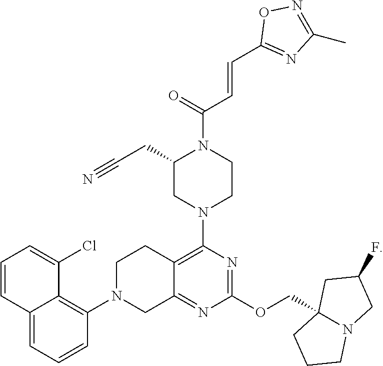 [SMILES: Cc1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1]

Compound 51 is a mixture of stereoisomers wherein the stereochemistry at the methylenenitrile on the piperazine is absolute as shown, the stereochemistry at the fluoropyrolizine is absolute as shown and stereocenter on the the azetidine group is a mixture of R and S.

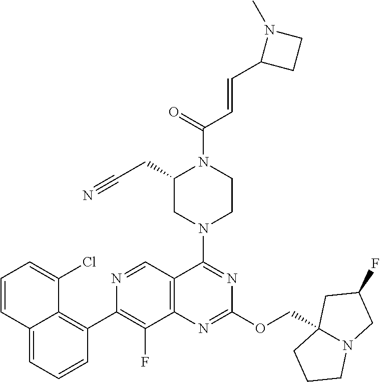 [SMILES: CN1CCC1/C=C/C(=O)N1CCN(c2nc(OC[C@@]34CCCN3C[C@H](F)C4)nc3c(F)c(-c4cccc5cccc(Cl)c45)ncc23)C[C@@H]1CC#N]

In some instances, the Stereochemistry column of Table 1 contains different indicators selected from (abs) (or) and (and) to refer to different stereocenters of the molecule. For example, Compound 15 includes a notation of “(abs) piperazine, (and) fluorocyclopropyl” in the stereochemistry column of Table 1.

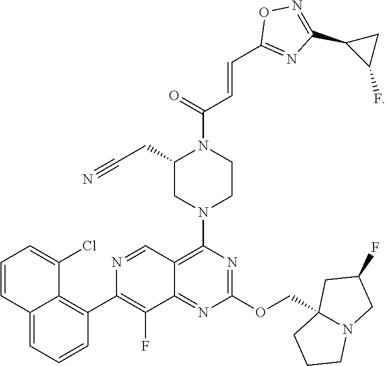 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/c1nc([C@H]2C[C@@H]2F)no1]

The compound is a a mixture of two stereoisomers, wherein the stereochemistry at the methylenenitrile on the piperazine is absolute as shown, the stereochemistry at the fluoropyrolizine is absolute as shown and the fluorocyclopropy group is a mixture of trans (R,S) and trans (S,R) isomers (prepared from a racemic trans fluorocyclopropyl intermediate).

A person of skill in the art would be able to separate racemic compounds into the respective enantiomers using methods known in the art, such as chiral chromatography, chiral recrystallization and the like. References to compounds that are racemic mixtures are meant to also include the individual enantiomers contained in the mixture.

Reference to “about” a value or parameter herein includes (and describes) variations that are directed to that value or parameter per se. For example, description referring to “about X” includes description of “Y”. As used herein, and unless otherwise specified, the terms “about” and “approximately,” when used in connection with temperatures, doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in this context, contemplate a dose, amount, or weight percent within 15%, within 10%, within 5%, within 4%, within 3%, within 2%, within 1%, or within 0.5% of the specified dose, amount, or weight percent.

The terms “a” and “an,” as used in herein mean one or more, unless context clearly dictates otherwise.

The terms “subject,” “individual,” and “patient” mean an individual organism, preferably a vertebrate, more preferably a mammal, most preferably a human. Examples of patients include humans, livestock such as cows, goats, sheep, pigs, and rabbits, and companion animals such as dogs, cats, and horses. In some embodiments, the subject has been identified or diagnosed as having a cancer or tumor having a KRAS G12C mutation (e.g., as determined using a regulatory agency-approved, e.g., FDA-approved, assay or kit).

“Treating” a disorder with the compounds and methods discussed herein is defined as administering one or more of the compounds discussed herein, with or without additional therapeutic agents, in order to reduce or eliminate either the disorder or one or more symptoms of the disorder, or to retard the progression of the disorder or of one or more symptoms of the disorder, or to reduce the severity of the disorder or of one or more symptoms of the disorder.

“Suppression” of a disorder with the compounds and methods discussed herein is defined as administering one or more of the compounds discussed herein, with or without additional therapeutic agents, in order to suppress the clinical manifestation of the disorder, or to suppress the manifestation of adverse symptoms of the disorder. The distinction between treatment and suppression is that treatment occurs after adverse symptoms of the disorder are manifest in a subject, while suppression occurs before adverse symptoms of the disorder are manifest in a subject. Suppression may be partial, substantially total, or total. In some embodiments, genetic screening can be used to identify patients at risk of the disorder. The compounds and methods disclosed herein can then be administered to asymptomatic patients at risk of developing the clinical symptoms of the disorder, in order to suppress the appearance of any adverse symptoms.

“Therapeutic use” of the compounds discussed herein is defined as using one or more of the compounds discussed herein to treat or suppress a disorder, as defined herein. A “therapeutically effective amount” of a compound is an amount of the compound, which, when administered to a subject, is sufficient to reduce or eliminate either the disorder or one or more symptoms of the disorder, or to retard the progression of the disorder or of one or more symptoms of the disorder, or to reduce the severity of the disorder or of one or more symptoms of the disorder, or to suppress the clinical manifestation of a disorder, or to suppress the manifestation of adverse symptoms of a disorder. A therapeutically effective amount can be given in one or more administrations.

A “KRAS G12C mediated cancer” is used interchangeably herein with a “cancer characterized by KRAS G12C”, and indicates that the cancer comprises cells which contain the KRAS G12C mutant.

While the compounds described herein can occur and can be used as the neutral (non-salt) compound, the description is intended to embrace all salts of the compounds described herein, as well as methods of using such salts of the compounds. In some embodiments, the salts of the compounds comprise pharmaceutically acceptable salts.

A “pharmaceutically acceptable salt” of a compound means a salt that is pharmaceutically acceptable to humans and/or animals, and which, upon administration, retains at least some of the desired pharmacological activity of the parent compound. Such salts include: (a) acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (b) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. Additional information on suitable pharmaceutically acceptable salts can be found in Remington's Pharmaceutical Sciences, 17th ed., Mack Publishing Company, Easton, PA, 1985, which is incorporated herein by reference in its entirety.

Included herein, when chemically relevant, are all stereoisomers of the compounds, including diastereomers and enantiomers. Also included are mixtures of possible stereoisomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

“Isotopologue” refers herein to a compound which differs in its isotopic composition from its “natural” isotopic composition. “Isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition. The description of compounds herein also includes all isotopologues, in some embodiments, partially deuterated or perdeuterated analogs, of all compounds herein. “Isotopically enriched” may also refer to a compound containing at least one atom having an isotopic composition other than the natural isotopic composition of that atom. “Isotopic enrichment” refers to the percentage of incorporation of an amount of a specific isotope at a given atom in a molecule in the place of that atom's natural isotopic abundance. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Alkyl” means a linear, branched, cyclic, or a combination thereof, saturated monovalent hydrocarbon radical having the defined number of carbons. For example, C₁-C₄ alkyl includes e.g., methyl, ethyl, propyl, 2-propyl, butyl, cyclopropyl, cyclobutyl, and the like. In one embodiment, alkyl is a linear or branched monovalent hydrocarbon radical having the defined number of carbons (“acyclic alkyl”). In one embodiment, alkyl is a cyclic monovalent hydrocarbon radical having the defined number of carbons (“cycloalkyl”).

“Alkylene” means a linear, branched, cyclic, or a combination thereof, saturated divalent hydrocarbon radical having the defined number of carbons. For example, C₁-C₄ alkylene includes e.g., methylene, ethylene, propylene, 1-methylpropylene, 2-methylpropylene, butylene, and the like. In one embodiment, alkylene is a linear or branched divalent hydrocarbon radical having the defined number of carbons (“acyclic alkylene”). In one embodiment, alkylene is a cyclic divalent hydrocarbon radical having the defined number of carbons (“cycloalkylene”). “C₀ alkylene” is means a bond. For example, C₀-C₂ alkylene includes a bond, methylene, ethylene, and the like.

“Alkenyl” means a linear or branched monovalent hydrocarbon radical containing one or more double bonds and having the defined number of carbons. For example, C₂-C₄ alkenyl includes e.g., vinyl, prop-1-en-2-yl, prop-1-en-1-yl, allyl and the like.

“Alkynyl” means a linear or branched monovalent hydrocarbon radical containing one or more triple bonds and having the defined number of carbons. For example, C₂-C₄ alkyne includes e.g., ethynyl, propynyl, 2-propynyl, butynyl, and the like.

“Alkoxy” means an —OR^{x} radical where R^{X} is alkyl as defined above, or a —R^{x′}OR^{x″} radical where R^{x′} is an alkylene and R^{x″} is an alkyl group as defined above where the defined number of alkyl carbons in the alkoxy group are equal to the total number of carbons in R^{x′} and R^{x″}. For example, C₁-C₄ alkoxy indicates e.g., methoxy, ethoxy, propoxy, 2-propoxy, n-, iso-, tert-butoxy, cyclopropoxy, methoxymethyl, ethoxymethyl, propoxymethyl, isopropoxymethyl, and the like. In some embodiments, alkoxy is a —OR^{x} radical. In some embodiments, alkoxy is a —R^{x′}OR^{x″} radical. In some embodiments, when a nitrogen is substituted with an alkoxy group, the alkoxy group is not linked to the nitrogen via the oxygen or a carbon that is immediately adjacent to the oxygen in the alkoxy group. For example, the alkoxy-substituted nitrogen is not N—OR or N—CH₂—O—R^{x″}.

“Alkoxyalkoxy” means an —OR^{y} radical where R^{y} is alkoxy as defined above, provided that the attachment point of R is not an oxygen atom, or a —R^{y′}OR^{y″} radical where R^{y′} is an alkylene and R^{y″} is an alkoxy group as defined above, provided that the attachment point of R^{y″} is not an oxygen atom, where the defined number of alkyl carbons in the alkoxyalkoxy group are equal to the total number of carbons in R^{y′} and R^{y″}. For example, C₁-C₆ alkoxyalkoxy indicates e.g., —OCH₂OCH₃, —OCH₂CH₂OCH₃, —OCH₂CH₂OCH₃, —CH₂OCH₂OCH₃, —CH₂OCH₂CH₂OCH₃, —CH₂OCH₂CH₂OCH₂CH₃, —CH₂CH₂OCH₂CH₂OCH₂CH₃ and the like. In some embodiments, alkoxyalkoxy is a —OR^{y} radical. In some embodiments, alkoxyalkoxy is a —R^{y′}OR^{y″} radical. In some embodiments, when a nitrogen is substituted with an alkoxyalkoxy group, the alkoxyalkoxy group is not linked to the nitrogen via the oxygen or a carbon that is immediately adjacent to the oxygen in the alkoxyalkoxy group. For example, the alkoxyalkoxy-substituted nitrogen is not N—O^{y}R or N—CH₂—O—R^{y″}.

“Aminoalkyl” means an —NHR^{z} radical where R^{z} is alkyl as defined above, or a —NR^{z}R^{z′} radical where R^{z} and R^{z′} are alkyl groups as defined above, or an —R^{z″}NH₂ radical where R^{z″} is an alkylene group as defined above, or an —R^{z″}NHR^{z} radical where R^{z″} is an alkylene group as defined above and R^{z′} is an alkyl group as defined above, or a —R^{z″}NR^{z}R^{z′} radical where R^{z″} is an alkylene group as defined above and R^{z} and R^{z′} are alkyl groups as defined above, where the defined number of alkyl carbons in the aminoalkyl group is equal to the total number of carbons in R^{z}, R^{z′} and R^{z″} as applicable. For example, C₁-C₆ aminoalkyl indicates e.g., —NHCH₃, —NHCH₂CH₃, —NHCH₂(CH₃)₂, —N(CH₃)₂, —N(CH₃)CH₂CH₃, —N(CH₂CH₃)₂, —CH₂NH₂, —CH₂CH₂NH₂, —CH₂NHCH₃, —CH₂N(CH₃)₂, —CH₂CH₂NHCH₃, —CH₂CH₂N(CH₃)₂ and the like. In some embodiments, aminoalkyl is an —NHR^{z} radical. In some embodiments, aminoalkyl is an —NR^{z}R^{z′} radical. In some embodiments, an aminoalkyl is an —R^{z″}NH₂ radical. In some embodiments, aminoalkyl is a —R^{z″}NHR^{z} radical. In some embodiments, aminoalkyl is a —R^{z″}NR^{z}R^{z′} radical. In some embodiments, when an oxygen is substituted with an aminoalkyl group, the aminoalkyl group is not linked to the oxygen via the nitrogen or a carbon that is immediately adjacent to the nitrogen in the aminoalkyl group. For example, the aminoalkyl-substituted oxygen is not O—NHR^{z} or O—CH₂—NHR^{z}.

“Aryl” refers to a radical of a monocyclic or polycyclic (e.g., bicyclic or tricyclic) aromatic ring system having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). In some embodiments, “aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryl groups include phenyl and naphthyl, wherein the attachment point can be on any carbon atom. Exemplary aryl groups also include indenyl, tetrahydronaphthyl, indolinyl, benzodihydrofuranyl, tetrahydroquinolinyl, tetrahydroisoquinolinyl and the like, wherein the attachment point is on the phenyl group. In some embodiments, “aryl” excludes ring systems wherein the aryl ring, as defined above, is fused with one or more carbocyclyl or heterocyclyl groups.

“Cycloalkyl” means a monocyclic saturated monovalent hydrocarbon radical having the defined number of carbon atoms. For example, C₃-C₆ cycloalkyl includes cyclopropyl, cyclobutyl, cyclopentyl, and cyclohexyl.

“Cyanoalkyl” means an alkyl radical as defined above, which is substituted with a cyano group (—CN). A cyanoalkyl can also be referred to as an alkylnitrile.

“Halo” means fluoro, chloro, bromo, or iodo. In some embodiments, halo is fluoro or chloro.

“Haloalkyl” means an alkyl radical as defined above, which is substituted with one or more halogen atoms, e.g., one to five halogen atoms, such as fluorine or chlorine, including those substituted with different halogens, e.g., —CH₂Cl, —CF₃, —CHF₂, —CH₂CF₃, —CF₂CF₃,

—CF(CH₃)₂, and the like. When the alkyl is substituted with only fluoro, it can be referred to in this Application as fluoroalkyl.

“Haloalkoxy” means an —OR^{a} radical where R^{a} is haloalkyl as defined above, or a —R^{b}OR^{c} radical where R^{b} and R^{c} are alkyl or haloalkyl groups as defined above where the defined number of alkyl carbons in the haloalkoxy group are equal to the total number of carbons in R^{b} and R^{c}. Halo atom(s) may be present in R^{b}, or R^{c}, or both, provided that at least one of R^{b} and R^{c} comprises a halo atom. For example, C₁-C₄ haloalkoxy indicates e.g., —OCF₃, —OCHF₂, —CH₂OCF₃, —CH₂CH(F)CH₂OCH₃, —CH₂CH(F)CH₂OCHF₂, and the like. In some embodiments, haloalkoxy is a —OR^{a} radical. In some embodiments, haloalkoxy is a —R^{b}OR^{c} radical. When all of the halo atom(s) in the haloalkoxy group are fluoro, it can be referred to in this Application as fluoroalkoxy. In some embodiments, when a nitrogen is substituted with a haloalkoxy group, the haloalkoxy group is not linked to the nitrogen via the oxygen or a carbon that is immediately adjacent to the oxygen in the haloalkoxy group. For example, the haloalkoxy-substituted nitrogen is not N—OR^{a} or N—C(H)ₙ(X)ₘ—O—R″.

“Hydroxyalkyl” means an alkyl radical as defined above, which is substituted with one or more hydroxyl (—OH) groups, e.g., one to three hydroxyl groups, e.g., —CH₂OH, —CH₂CH₂OH, —C(OH)(CH₃)₂, —CH(OH)CH₃ and the like.

A “heterocyclic group” or “heterocycle”, unless otherwise specified, means a saturated or partially unsaturated cyclic group comprising 3-12 ring atoms, in which 1-4 ring atoms are heteroatoms independently selected from the group consisting of nitrogen, oxygen, and sulfur, the remaining rings being C. The sulfur group may be present either as —S— or as —S(O)₂—. Unless otherwise specified, the heterocyclic group includes single as well as multiple ring systems including fused, bridged, and spiro ring systems. “Heterocyclic group” or “heterocycle” also includes ring systems wherein the heterocyclic group, as defined above, is fused with one or more carbocyclic groups wherein the point of attachment is either on the carbocycle or heterocycle ring In some embodiments, “heterocyclic group” or “heterocycle” also includes ring systems wherein the heterocyclic group, as defined above, is fused with one or more aryl or heteroaryl groups, wherein the point of attachment is on the heterocyclyl ring, and in such instances, the number of ring members continue to designate the number of ring members in the heterocyclyl ring system. In some embodiments, the heterocyclic group is a single ring. In some embodiments, the heterocyclic group comprises two fused rings. In some embodiments, the heterocyclic group comprises two spiro rings. In some embodiments, the heterocyclic group comprises a bridged ring system.

A “carbocyclic group” or carbocycle”, unless otherwise specified, means a saturated or partially unsaturated cyclic group comprising 3-12 ring atoms, in which the ring atoms are C. Unless otherwise specified, the carbocyclic group includes single as well as multiple ring systems including fused, bridged, and spiro ring systems. In some embodiments, the carbocyclic group is a single ring. In some embodiments, the carbocyclic group comprises two fused rings. In some embodiments, the carbocyclic group comprises two spiro rings. In some embodiments, the carbocyclic group comprises a bridged ring system.

“Heteroaryl” means a monovalent monocyclic or bicyclic aromatic radical of 5 to 10 ring atoms, unless otherwise stated, where one or more (in some embodiments, one, two, or three) ring atoms are heteroatom(s) independently selected from N, O, or S, the remaining ring atoms being carbon. In some embodiments, “heteroaryl” includes ring systems wherein the heteroaryl ring, as defined above, is fused with one or more carbocyclyl or heterocyclyl groups wherein the point of attachment is on the heteroaryl ring, In such instances, unless otherwise specified, the number of ring members continue to designate the number of ring members in the heteroaryl ring system. In some embodiments, “heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). In some embodiments, “heteroaryl” excludes ring systems wherein the heteroaryl ring is fused with a carbocyclyl or heterocyclyl group.

Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

A “spiro” cycloalkyl group indicates that the cycloalkyl group is linked to the remaining portion of the compound through a spiro linkage. A “spiro” cycloalkyl substituent has two attachments that connect to the same carbon of the moiety that is substituted, forming a spiro connection. For example, a cyclohexyl group that is substituted with a spiro cyclopropyl group indicates:

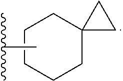

“In need of treatment” as used herein means the patient is being treated by a physician or other caregiver after diagnoses of the disease, or a determination that the patient is at risk for developing the disease. In some embodiments, the patient has been diagnosed as having a KRAS G12C mediated cancer. In some embodiments, the patient has been determined to be at risk of developing a KRAS G12C mediated cancer.

“Administration”, “administer” and the like, as they apply to, for example, a patient, cell, tissue, organ, or biological fluid, refer to contact of, for example, a compound of Formula (A), Formula (B) or Formula (C), or a pharmaceutically acceptable salt and/or isotopologue thereof, a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

“Optional” or “optionally” means that the subsequently described event or circumstance may but need not occur, and that the description includes instances where the event or circumstance occurs and instances in which it does not.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a disease or disorder described in the present disclosure. Such administration encompasses co-administration of these therapeutic agents in a substantially simultaneous manner, such as in a single capsule or a tablet having a fixed ratio of active ingredients or in multiple, separate capsules or tablets for each active ingredient. In addition, such administration also encompasses use of each type of therapeutic agent in a sequential manner. In either case, the treatment regimen will provide beneficial effects of the drug combination in treating the conditions or disorders described herein.

### Compounds

In one aspect, the invention provides compound of Formula (A), Formula (B) or Formula (C)

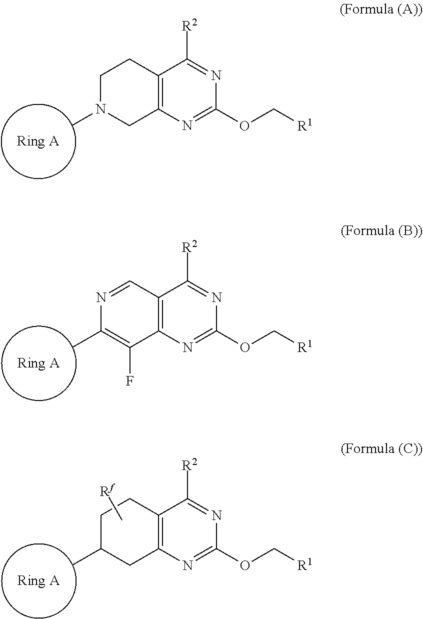

or a salt thereof; and/or an isotopologue thereof, wherein:

Ring A is a 6-10 membered aryl or a 5-10 membered heteroaryl, each substituted with 0, 1, 2 or 3 substituents independently selected from halo, —OH, —NH₂, C₁-C₄ alkyl, C₁-C₄ alkenyl, C₁-C₄ alkoxy, C₁-C₄ haloalkyl, C₁-C₄ haloalkoxy and C₂-C₃ alkynyl;
R¹ is

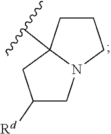

R^{d} is H, —F or —OH;
R² is R^{2c};
R^{2c} is

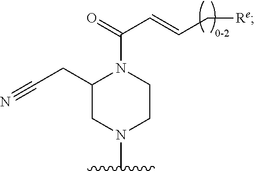

R^{e} is R^{e1}, R^{e2} or R^{e3};
R^{e1} is a 4-10 membered heterocycle which is substituted with 0, 1, 2, 3 or 4 substituents independently selected from halo, hydroxy, C₁-C₄ alkyl, C₁-C₆ aminoalkyl, 4-6 membered heterocycle, —C(O)C₁-C₆ alkyl, C₁-C₆ alkoxy, C₁-C₆ alkoxyalkoxy, C₁-C₄ haloalkyl, C₁-C₄ haloalkoxy and C₂-C₃ alkynyl;
R^{e2} is a 5-6 membered heteroaryl wherein the attachment point is a carbon atom group and wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl;
R^{e3} is —NR⁵R⁶, wherein each R⁵ and R⁶ is independently selected from C₁-C₄ alkyl, C₁-C₆ alkoxy and —CH₂-(4-6 membered heterocycle); and
R^{f} is selected from the group consisting of H, halo, C₁-C₄ alkyl, C₁-C₄ alkoxy, C₁-C₄ haloalkyl and C₁-C₄ haloalkoxy.

Throughout the description, references to compounds of Formula (A), Formula (B) and Formula (C) are meant to encompass all subgenera and subcombinations of those formulae described herein, including compounds of Formula I, Formula II, Formula III, Formula IV, Formula V, Formula VI, Formula VII, Formula VIII, Formula IX, Formula IXa as well as compounds of Table 1.

In an embodiment, the compound of Formula (A), Formula (B) or Formula (C) as described in any of the embodiments described herein is not a salt. In an embodiment, the compound of Formula (A), Formula (B) or Formula (C) as described in any of the embodiments described herein is a salt. In an embodiment, the salt is a formate salt. In an embodiment, the salt is a trifluoroacetate salt. In an embodiment, the salt is a pharmaceutically acceptable salt.

In an embodiment, provided is a compound of Formula (A), Formula (B) or Formula (C)

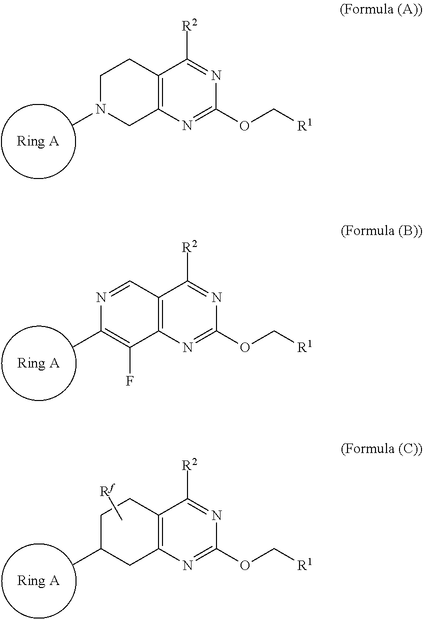

or a salt thereof; and/or an isotopologue thereof, wherein:
Ring A is a 6-10 membered aryl or a 5-10 membered heteroaryl, each substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl;
R¹ is

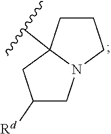

R^{d} is H or F;
R² is R^{2c};
R^{2c} is

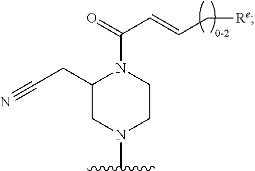

R^{e} is R^{e1} or R^{e2};
R^{e1} is a 4-10 membered heterocycle which is substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl;
R^{e2} is a 5-6 membered heteroaryl wherein the attachment point is a carbon atom group and wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl; and
R^{f} is selected from the group consisting of H, halo, C₁-C₄ alkyl, C₁-C₄ alkoxy, C₁-C₄ haloalkyl and C₁-C₄ haloalkoxy.

In an embodiment, the compound is of Formula (A) or Formula (B). In an embodiment, the compound is of Formula (A) or Formula (C). In an embodiment, the compound is of Formula (B) or Formula (C). In an embodiment, the compound is of Formula (A). In an embodiment, the compound is of Formula (B). In an embodiment, the compound is of Formula (C).

As generally defined herein, Ring A is a 6-10 membered aryl or a 5-10 membered heteroaryl, each substituted with 0, 1, 2 or 3 substituents independently selected from halo, —OH, —NH₂, C₁-C₄ alkyl, C₁-C₄ alkenyl, C₁-C₄ alkoxy, C₁-C₄ haloalkyl, C₁-C₄ haloalkoxy and C₂-C₃ alkynyl. In an embodiment, the 6-10 membered aryl or the 5-10 membered heteroaryl of Ring A is unsubstituted. In an embodiment, the 6-10 membered aryl or the 5-10 membered heteroaryl of Ring A is substituted with 1 substituent as described above. In an embodiment, the 6-10 membered aryl or the 5-10 membered heteroaryl of Ring A is substituted with 2 substituents as described above. In an embodiment, the 6-10 membered aryl or the 5-10 membered heteroaryl of Ring A is substituted with 3 substituents as described above.

In an embodiment, Ring A is selected from the group consisting of naphthalenyl (e.g., naphthalen-1-yl), phenyl, isoquinolinyl (e.g., isoquinolin-1-yl), pyridinyl (e.g., pyridin-2-yl) and 1-H-indazolyl (e.g., 1-H-indazol-3-yl, 1-H-indazol-4-yl), each substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl.

In an embodiment, the naphthalenyl (e.g., naphthalen-1-yl), phenyl, isoquinolinyl (e.g., isoquinolin-1-yl), pyridinyl (e.g., pyridin-2-yl) and 1-H-indazolyl (e.g., 1-H-indazol-3-yl, 1-H-indazol-4-yl) are independently substituted with 0, 1, 2 or 3 substituents independently selected from halo, —NH₂, C₁-C₄ acyclic alkyl, C₃-C₄ cycloalkyl, C₁-C₄ alkenyl, C₁-C₄ haloalkyl and C₂-C₃ alkynyl.

In an embodiment, the naphthalenyl (e.g., naphthalen-1-yl), phenyl, isoquinolinyl (e.g., isoquinolin-1-yl), pyridinyl (e.g., pyridin-2-yl) and 1-H-indazolyl (e.g., 1-H-indazol-3-yl, 1-H-indazol-4-yl) are independently substituted with 0, 1, 2 or 3 substituents independently selected from —F, —Cl, —OH, —NH₂, -Me, -Et, —Pr, —ⁱPr, cyclopropyl, cyclobutyl, vinyl, prop-1-en-2-yl, —CHF₂, —CH₂F, —CF₃, —OMe, —OEt, —OCHF₂, —OCF₃ and ethynyl.

In an embodiment, the naphthalenyl (e.g., naphthalen-1-yl), phenyl, isoquinolinyl (e.g., isoquinolin-1-yl), pyridinyl (e.g., pyridin-2-yl) and 1-H-indazolyl (e.g., 1-H-indazol-3-yl, 1-H-indazol-4-yl) are independently substituted with 0, 1, 2 or 3 substituents independently selected from —F, —Cl, —NH₂, -Me, -Et, cyclopropyl, cyclobutyl, prop-1-en-2-yl, —CHF₂, —CF₃, and ethynyl.

In an embodiment, Ring A is selected from the group consisting of naphthalen-1-yl, phenyl, isoquinolin-1-yl, pyridin-2-yl, 1-H-indazol-3-yl and 1-H-indazol-4-yl, each substituted as described in any of the embodiments described herein.

In an embodiment, Ring A is selected from the group consisting of naphthalenyl (e.g., naphthalen-1-yl), phenyl and pyridinyl (e.g., pyridin-2-yl), each substituted as described in any of the embodiments described herein.

In an embodiment, Ring A is selected from the group consisting of naphthalenyl (e.g., naphthalen-1-yl), phenyl, isoquinolinyl (e.g., isoquinolin-1-yl) and pyridinyl (e.g., pyridin-2-yl), each substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl.

In an embodiment of any of the above embodiments, the naphthalenyl is naphthalen-1-yl. In an embodiment any of the above embodiments, the pyridinyl is pyridin-2-yl. In an embodiment any of the above embodiments, the isoquinolinyl is isoquinolin-1-yl. In an embodiment of any of the above embodiments the 1-H-indazolyl is 1-H-indazol-3-yl or 1-H-indazol-4-yl. In an embodiment the 1-H-indazolyl is 1-H-indazol-3-yl. In an embodiment the 1-H-indazolyl is 1-H-indazol-4-yl.

In an embodiment, Ring A is selected from the group consisting of naphthalen-1-yl, phenyl, isoquinolin-1-yl and pyridin-2-yl, each substituted as described in any of the embodiments described herein. In an embodiment, Ring A is selected from the group consisting of naphthalen-1-yl and phenyl, each substituted as described in any of the embodiments described herein.

In an embodiment each of the naphthalenyl (e.g., naphthalen-1-yl), phenyl, isoquinolinyl (e.g., isoquinolin-1-yl), pyridinyl (e.g., pyridin-2-yl) and 1-H-indazolyl (e.g., 1-H-indazol-3-yl, 1-H-indazol-4-yl) groups of Ring A can be unsubstituted. In an embodiment, each of the naphthalenyl (e.g., naphthalen-1-yl), phenyl, isoquinolinyl (e.g., isoquinolin-1-yl), pyridinyl (e.g., pyridin-2-yl) and 1-H-indazolyl (e.g., 1-H-indazol-3-yl, 1-H-indazol-4-yl) of ring A is substituted with 1 substituent independently selected from the substituents described in any of the embodiments described above. In an embodiment, each of the naphthalenyl (e.g., naphthalen-1-yl), phenyl, isoquinolinyl (e.g., isoquinolin-1-yl), pyridinyl (e.g., pyridin-2-yl) and 1-H-indazolyl (e.g., 1-H-indazol-3-yl, 1-H-indazol-4-yl) of ring A is substituted with 2 substituents independently selected from the substituents described in any of the embodiments described above. In an embodiment, each of the naphthalenyl (e.g., naphthalen-1-yl), phenyl, isoquinolinyl (e.g., isoquinolin-1-yl), pyridinyl (e.g., pyridin-2-yl) and 1-H-indazolyl (e.g., 1-H-indazol-3-yl, 1-H-indazol-4-yl) of ring A is substituted with 3 substituents independently selected from the substituents described in any of the embodiments described above.

In an embodiment, Ring A is naphthalenyl (e.g., naphthalen-1-yl) substituted with 0, 1, 2 or 3 substituents independently selected from halo, hydroxy, C₁-C₄ alkyl, C₁-C₄ alkoxy, C₁-C₄ haloalkyl, C₁-C₄ haloalkoxy and C₂-C₃ alkynyl. In an embodiment, the naphthalenyl (e.g., naphthalen-1-yl) is substituted with 0, 1 or 2 substituents independently selected from halo, C₁-C₄ acyclic alkyl and C₂-C₃ alkynyl. In an embodiment, the naphthalenyl (e.g., naphthalen-1-yl) is substituted with 0, 1 or 2 substituents independently selected from —F, —Cl, -Me, -Et and ethynyl. In an embodiment, the naphthalenyl (e.g., naphthalen-1-yl) is unsubstituted. In an embodiment, the naphthalenyl (e.g., naphthalen-1-yl) is substituted with 1 or 2 substituents independently selected from —F, —Cl, -Et and ethynyl. In an embodiment, the naphthalenyl (e.g., naphthalen-1-yl) is substituted with 1 or 2 substituents independently selected from —F and —Cl. In an embodiment, the naphthalenyl (e.g., naphthalen-1-yl) is substituted with 1 or 2 instances of —F. In an embodiment, the naphthalenyl (e.g., naphthalen-1-yl) is substituted with 1 or 2 instances of —Cl. In an embodiment, the naphthalenyl is naphthalen-1-yl.

In an embodiment, Ring A is selected from the group consisting of:

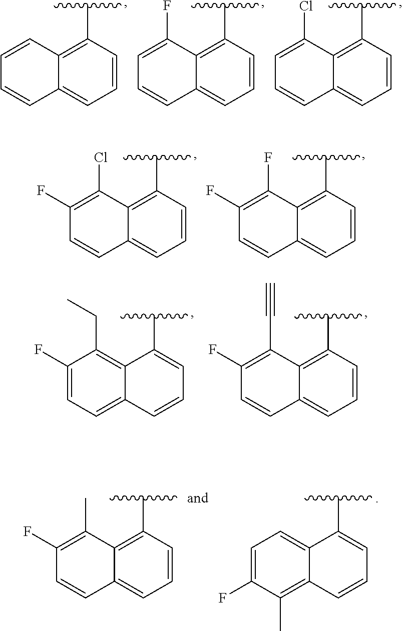

In an embodiment, Ring A is selected from the group consisting of:

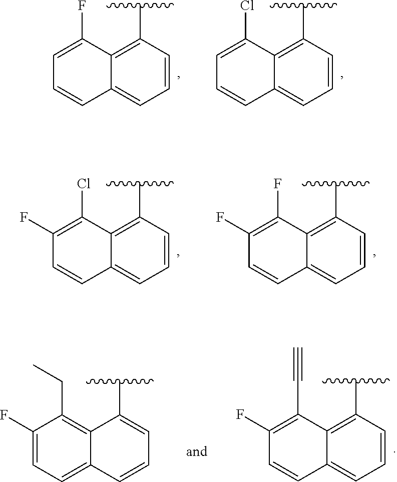 [SMILES: *c1cccc2cccc(F)c12 ; *c1cccc2cccc(Cl)c12 ; *c1cccc2ccc(F)c(Cl)c12 ; *c1cccc2ccc(F)c(F)c12 ; *c1cccc2ccc(F)c(CC)c12 ; *c1cccc2ccc(F)c(C#C)c12]

In an embodiment, Ring A is selected from the group consisting of:

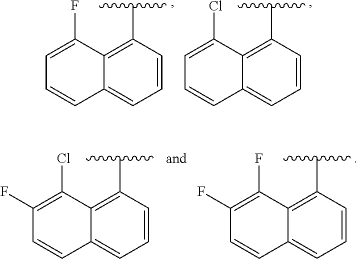

In an embodiment, Ring A is

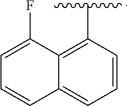 [SMILES: *c1cccc2cccc(F)c12 ; "."]

In an embodiment, Ring A is

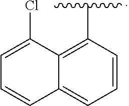 [SMILES: *c1cccc2cccc(Cl)c12 ; "."]

In an embodiment, Ring A is

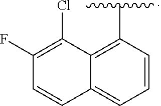 [SMILES: *c1cccc2ccc(F)c(Cl)c12 ; "."]

In an embodiment, Ring A is

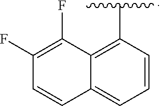 [SMILES: *c1cccc2ccc(F)c(F)c12 ; "."]

In an embodiment, Ring A is

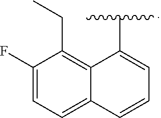 [SMILES: *c1cccc2ccc(F)c(CC)c12 ; "."]

In an embodiment, Ring A is

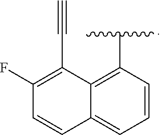 [SMILES: *c1cccc2ccc(F)c(C#C)c12 ; "."]

In an embodiment, Ring A is isoquinolinyl (e.g., isoquinolinyl-1-yl) substituted with 0, 1, 2 or 3 substituents independently selected from halo, —OH, —NH₂, C₁-C₄ alkyl, C₁-C₄ alkoxy, C₁—C₄ haloalkyl, C₁-C₄ haloalkoxy and C₂-C₃ alkynyl. In an embodiment, the isoquinolinyl (e.g., isoquinolinyl-1-yl) is substituted with 1, 2 or 3 substituents independently selected from halo and —NH₂. In an embodiment, the isoquinolinyl (e.g., isoquinolinyl-1-yl) is substituted with 1 or 2 substituents independently selected from —F, —Cl and —NH₂.

In an embodiment of the embodiments described above, the isoquinolinyl is isoquinolinyl-1-yl.

In an embodiment, Ring A is selected from the group consisting of

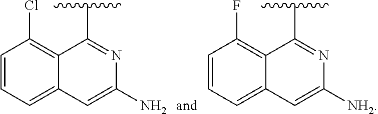 [SMILES: *c1nc(N)cc2cccc(Cl)c12 ; "and" ; *c1nc(N)cc2cccc(F)c12]

In an embodiment, Ring A is

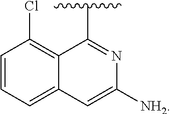 [SMILES: *c1nc(N)cc2cccc(Cl)c12]

In an embodiment, Ring A is

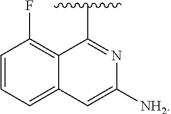 [SMILES: *c1nc(N)cc2cccc(F)c12]

In an embodiment, Ring A is phenyl substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl. In an embodiment, Ring A is phenyl substituted with 0, 1, 2 or 3 substituents independently selected from halo, —OH, —NH₂, C₁-C₄ alkyl, C₁-C₄ alkenyl, C₁-C₄ alkoxy, C₁-C₄ haloalkyl, C₁-C₄ haloalkoxy and C₂-C₃ alkynyl. In an embodiment, the phenyl is substituted with 1, 2 or 3 substituents independently selected from halo, —NH₂, C₁-C₄ acyclic alkyl, C₃-C₄ cycloalkyl, C₁-C₄ alkenyl and C₁-C₄ haloalkyl. In an embodiment, the phenyl is substituted with 1, 2 or 3 substituents independently selected from —F, —Cl, -Me, -Et, cyclopropyl, cyclobutyl, prop-1-en-2-yl, —CHF₂ and —CF₃. In an embodiment, the phenyl is substituted with 1 or 2 substituents independently selected from —F, —Cl, cyclopropyl and —CF₃. In an embodiment, the phenyl is substituted with 2 substituents independently selected from —F, —Cl, cyclopropyl and —CF₃.

In an embodiment, Ring A is selected from the group consisting of

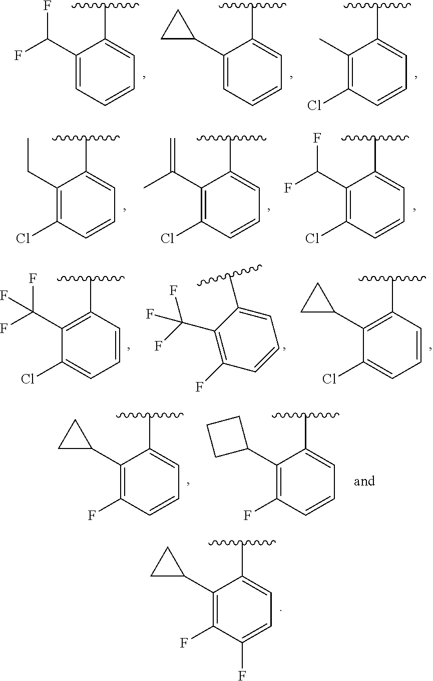

In an embodiment, Ring A is selected from the group consisting of

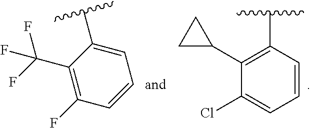 [SMILES: *c1cccc(F)c1C(F)(F)F ; "and" ; *c1cccc(Cl)c1C1CC1 ; "."]

In an embodiment, Ring A is

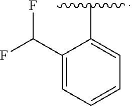 [SMILES: *c1ccccc1C(F)F ; "."]

In an embodiment Ring A is

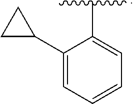 [SMILES: *c1ccccc1C1CC1 ; "."]

In an embodiment, Ring A is

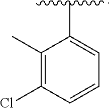 [SMILES: *c1cccc(Cl)c1C ; "."]

In an embodiment, Ring A is

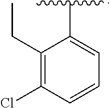 [SMILES: *c1cccc(Cl)c1CC ; "."]

In an embodiment, Ring A is

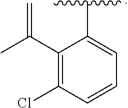 [SMILES: *c1cccc(Cl)c1C(=C)C ; "."]

In an embodiment, Ring A is

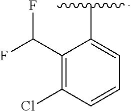 [SMILES: *c1cccc(Cl)c1C(F)F ; "."]

In an embodiment, Ring A is

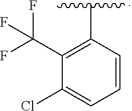 [SMILES: *c1cccc(Cl)c1C(F)(F)F ; "."]

In an embodiment, Ring A is

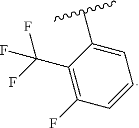 [SMILES: *c1cccc(F)c1C(F)(F)F ; "."]

In an embodiment, Ring A is

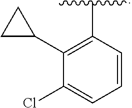 [SMILES: *c1cccc(Cl)c1C1CC1 ; "."]

In an embodiment, Ring A is

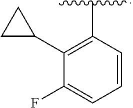 [SMILES: *c1cccc(F)c1C1CC1 ; "."]

In an embodiment, Ring A is

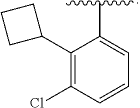 [SMILES: *c1cccc(Cl)c1C1CCC1 ; "."]

In an embodiment, Ring A is

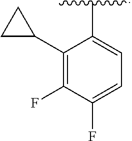 [SMILES: "." ; *c1ccc(F)c(F)c1C1CC1]

In an embodiment, Ring A is pyridinyl (e.g., pyridin-2-yl) substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl. In an embodiment, the pyridinyl (e.g., pyridin-2-yl) is substituted with 1, 2 or 3 substituents independently selected from —NH₂, C₁-C₄ acyclic alkyl and C₁-C₄ haloalkyl. In an embodiment, the pyridinyl (e.g., pyridin-2-yl) is substituted with 1, 2 or 3 substituents independently selected from —NH₂, -Me and —CF₃. In an embodiment of the embodiments described above, the pyridinyl is pyridin-2-yl. In an embodiment, Ring A is

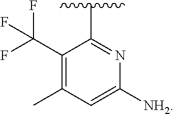 [SMILES: *c1nc(N)cc(C)c1C(F)(F)F]

In an embodiment, Ring A is 1-H-indazolyl (e.g., 1-H-indazol-3-yl, 1-H-indazol-4-yl) substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl. In an embodiment, the 1-H-indazolyl (e.g., 1-H-indazol-3-yl, 1-H-indazol-4-yl) is substituted with 1 or 2 substituents independently selected from halo and acyclic C₁-C₄ alkyl. In an embodiment, the 1-H-indazolyl (e.g., 1-H-indazol-3-yl, 1-H-indazol-4-yl) is substituted with 1 or 2 substituents independently selected from —F, —Cl and -Me. In an embodiment, of any of the above embodiments, the 1-H-indazolyl is selected from the group consisting of 1-H-indazol-3-yl and 1-H-indazol-4-yl. In an embodiment, the 1-H-indazolyl is 1-H-indazol-3-yl. In an embodiment, the 1-H-indazolyl is 1-H-indazol-4-yl. In an embodiment, Ring A is selected from

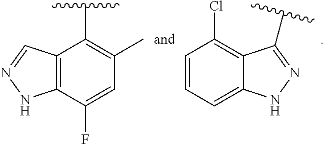

In an embodiment, Ring A is

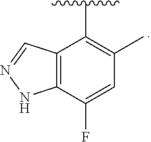 [SMILES: *c1c(C)cc(F)c2[nH]ncc12 ; "."]

In an embodiment, Ring A is

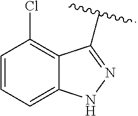 [SMILES: *c1n[nH]c2cccc(Cl)c12 ; "."]

In an embodiment, Ring A is selected from the group consisting of

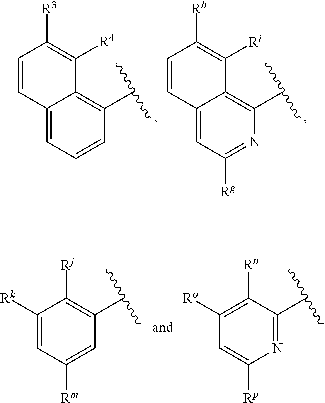

wherein each R³, R⁴, R^{g}, R^{h}, Rⁱ, R^{j}, R^{k}, R^{m}, Rⁿ, R^{o} and R^{p} are as defined in any of the embodiments described herein.

In an embodiment, Ring A is selected from the group consisting of

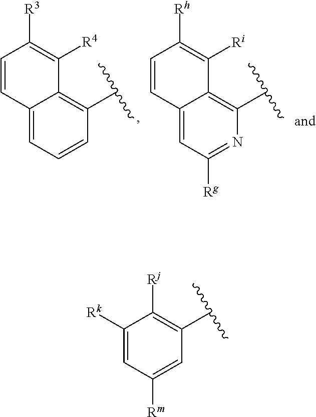

wherein each R³, R⁴, R^{g}, R^{h}, Rⁱ, R^{j}, R^{k} and R^{m} are as defined in any of the embodiments described herein.

In an embodiment, Ring A is selected from the group consisting of

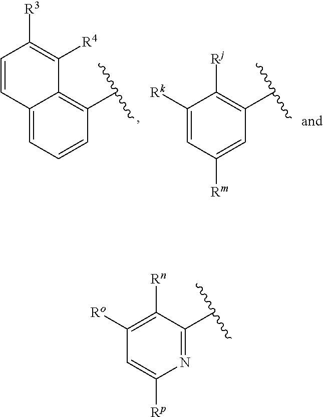

wherein each R³, R⁴, R^{j}, R^{k}, R^{m}, Rⁿ, R^{o} and R^{p} are as defined in any of the embodiments described herein.

In an embodiment, Ring A is selected from the group consisting of

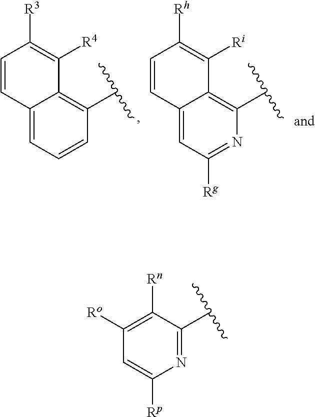

wherein each R³, R⁴, R^{g}, R^{h}, Rⁱ, Rⁿ, R^{o} and R^{p} are as defined in any of the embodiments described herein.

In an embodiment, Ring A is selected from the group consisting of

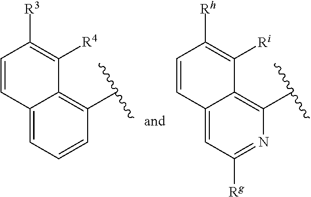

wherein each R³, R⁴, R⁹, R^{h} and Rⁱ are as defined in any of the embodiments described herein.

In an embodiment, Ring A is selected from the group consisting of

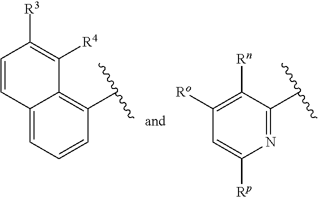

wherein each R³, R⁴, Rⁿ, R^{o} and R^{p} are as defined in any of the embodiments described herein.

In an embodiment, Ring A is selected from the group consisting of

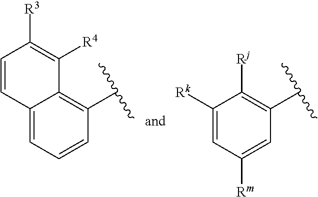

wherein each R³, R⁴, R^{j}, R^{k} and R^{m} are as defined in any of the embodiments described herein.

In an embodiment, Ring A is

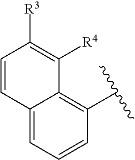

wherein each R³ and R⁴ are as defined in any of the embodiments described herein. In an embodiment, Ring A is

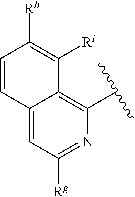

wherein each R^{g}, R^{h} and Rⁱ are as defined in any of the embodiments described herein.

In an embodiment, Ring A is

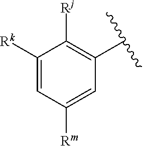

wherein each R^{j}, R^{k} and R are as defined in any of the embodiments described herein. In an embodiment, Ring A is

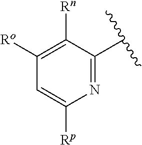

wherein each Rⁿ, R^{o} and R^{p} are as defined in any of the embodiments described herein.

As generally defined herein, R¹ is

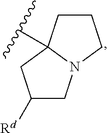

wherein R^{d} is as defined in any of the embodiments described herein. In an embodiment, the R^{d} group and the attachment to the methylene linker are in a trans configuration. In one embodiment, when R^{d} is not H, the stereochemistry at the quaternary carbon of R¹ is (S) and the stereochemistry at the carbon connected to R^{d} is (R). In an embodiment, R¹ is

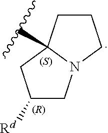

In an embodiment, R¹ is

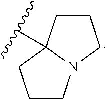 [SMILES: *C12CCCN1CCC2 ; "."]

In an embodiment, R¹ is

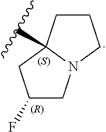

In an embodiment, R¹ is

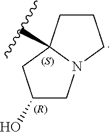

As generally defined herein, R² is R^{2c}, wherein

R^{2c} is

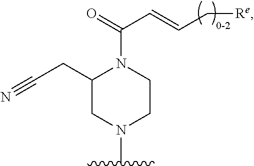

wherein R is as defined in any of the embodiments described herein.

In an embodiment, R^{2c} is selected from the group consisting of and

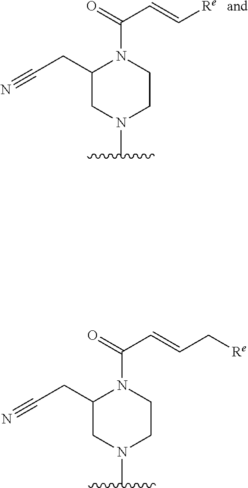

wherein R^{e} is as defined in any of the embodiments described herein.

In an embodiment, R^{2c} is

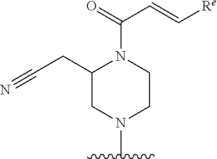

wherein R^{e} is as defined in any of the embodiments described herein. In an embodiment, R^{2c} is

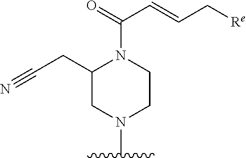

wherein R^{e} is as defined in any of the embodiments described herein. In an embodiment, R^{2c} is

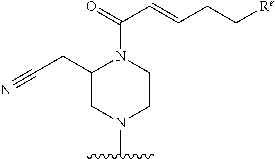

wherein R^{e} is as defined in any of the embodiments described herein.

In an embodiment, R^{2c} is selected from the group consisting of:

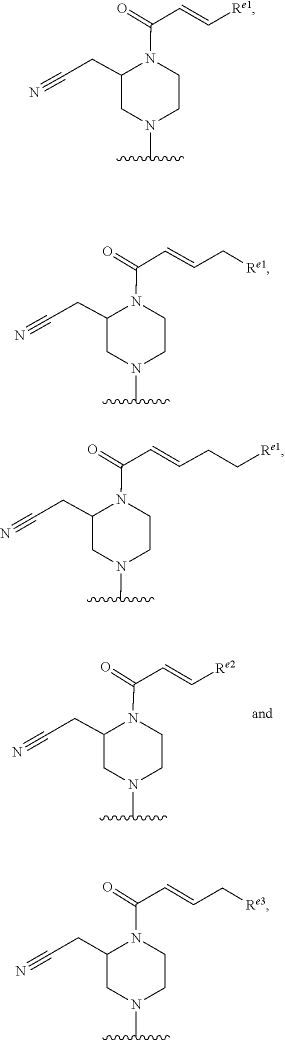

wherein R^{e1}, R^{e2} and R^{e3} are as defined in any of the embodiments described herein.

In an embodiment, R^{2c} is selected from the group consisting of:

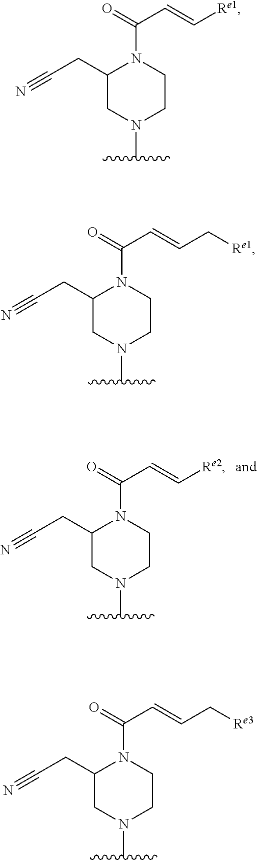

wherein R^{e1}, R^{e2} and R^{e3} are as defined in any of the embodiments described herein. In an embodiment, R^{2c} is

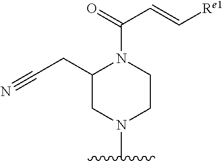

wherein R^{e1} is as defined in any of the embodiments described herein. In an embodiment, R^{2c} is

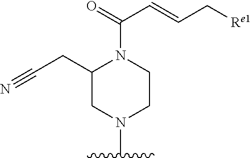

wherein R^{e1} is as defined in any of the embodiments described herein. In an embodiment, R^{2c} is

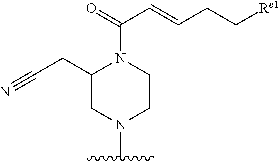

wherein R^{e1} is as defined in any of the embodiments described herein. In an embodiment, R^{2c} is

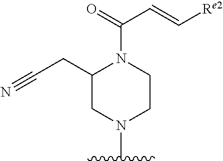

wherein R^{e2} is as defined in any of the embodiments described herein. In an embodiment, R^{2c} is

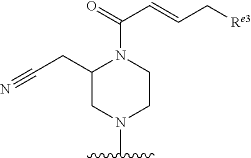

wherein R^{e3} is as defined in any of the embodiments described herein.

In an embodiment, the stereochemistry at the methylenenitrile group of R^{2c} is (S). In an embodiment, R^{2c} is

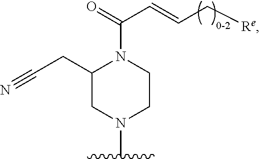

wherein R^{e} is as defined in any of the embodiments described herein.

In an embodiment, R^{2c} is selected from the group consisting of

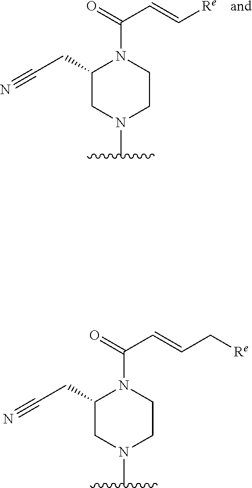

wherein R^{e} is as defined in any of the embodiments described herein.

In an embodiment, R^{2c} is

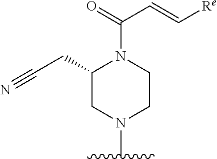

wherein R is as defined in any of the embodiments described herein. In an embodiment, R^{2c}

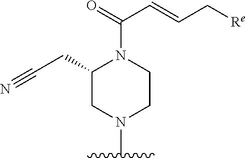

is wherein R^{e} is as defined in any of the embodiments described herein. In an embodiment, R^{2c} is

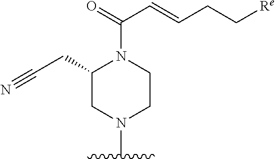

wherein R^{e} is as defined in any of the embodiments described herein.

In an embodiment, R^{2c} is selected from the group consisting of:

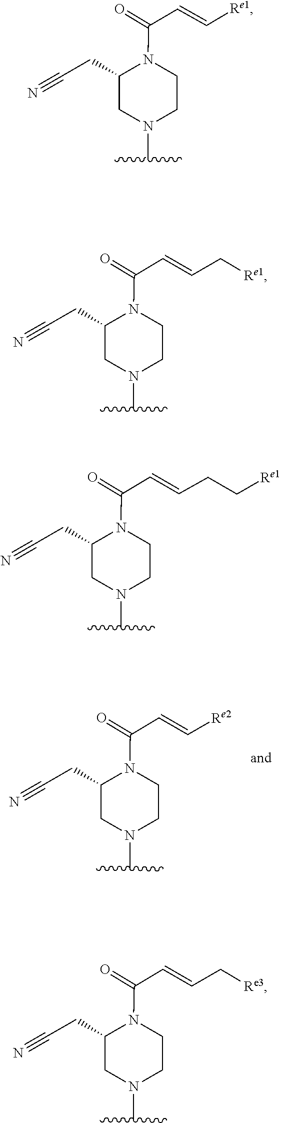

wherein R^{e1}, R^{e2} and R^{e3} are as defined in any of the embodiments described herein.

In an embodiment, R^{2c} is selected from the group consisting of:

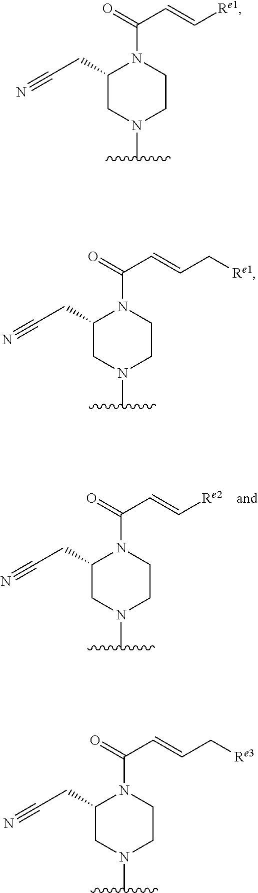

wherein R^{e1}, R^{e2} and R^{e3} are as defined in any of the embodiments described herein. In an embodiment, R^{2c} is

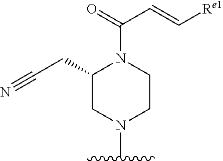

wherein R^{e1} is as defined in any of the embodiments described herein. In an embodiment, R^{2c}

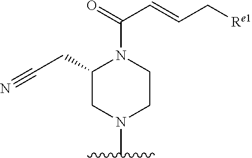

is wherein R^{e1} is as defined in any of the embodiments described herein. In an embodiment, R^{2c} is

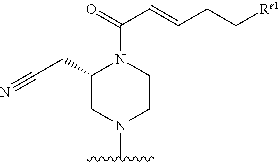

wherein R^{e1} is as defined in any of the embodiments described herein. In an embodiment, R^{2c} is

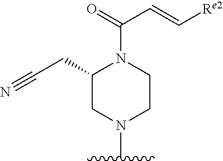

wherein R^{e2} is as defined in any of the embodiments described herein. In an embodiment, R^{2c}

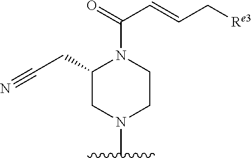

is wherein R^{e3} is as defined in any of the embodiments described herein.

As generally defined herein, each R³ is independently selected from the group consisting of hydrogen, halo, C₁-C₄ alkyl, C₁-C₄ alkoxy, C₁-C₄ haloalkyl, C₁-C₄ haloalkoxy, C₁-C₄ alkenyl and C₂-C₃ alkynyl. In an embodiment, each R³ is independently selected from the group consisting of hydrogen, halo, C₁-C₄ alkyl, C₁-C₄ alkoxy, C₁-C₄ haloalkyl, C₁-C₄ haloalkoxy and C₂-C₃ alkynyl. In an embodiment, R³ is selected from the group consisting of hydrogen, fluoro, and chloro. In an embodiment, R³ is selected from the group consisting of hydrogen, halo, C₁-C₄ alkyl and C₂-C₃ alkynyl. In an embodiment, R³ is selected from the group consisting of hydrogen, —F, —Cl, -Et and ethynyl. In an embodiment, R³ is selected from the group consisting of hydrogen and fluoro. In an embodiment, R³ is selected from the group consisting of hydrogen and —F. In an embodiment, R³ is —F. In an embodiment, R³ is hydrogen.

As generally defined herein, each R⁴ is independently selected from the group consisting of hydrogen, halo, C₁-C₄ alkyl, C₁-C₄ alkoxy, C₁-C₄ haloalkyl, C₁-C₄ haloalkoxy, C₁-C₄ alkenyl and C₂-C₃ alkynyl. In an embodiment, each R⁴ is independently selected from the group consisting of hydrogen, halo, C₁-C₄ alkyl, C₁-C₄ alkoxy, C₁-C₄ haloalkyl, C₁-C₄ haloalkoxy and C₂-C₃ alkynyl. In an embodiment, R⁴ is selected from the group consisting of hydrogen, fluoro, and chloro. In an embodiment, R⁴ is selected from the group consisting of hydrogen, halo, C₁-C₄ alkyl and C₂-C₃ alkynyl. In an embodiment, R⁴ is selected from the group consisting of hydrogen, —F, —Cl, -Et and ethynyl. In an embodiment, R⁴ is selected from the group consisting of hydrogen, —F, —Cl, -Et and ethynyl. In an embodiment, R⁴ is selected from the group consisting of —F, —Cl, -Et and ethynyl. In an embodiment, R⁴ is selected from the group consisting of —F and —Cl. In an embodiment, R⁴ is —F. In an embodiment, R⁴ is -Et and ethynyl. In an embodiment, R⁴ is ethynyl. In an embodiment, R⁴ is chloro. In an embodiment, R⁴ is hydrogen.

As generally defined herein, each R is independently selected from C₁-C₄ alkyl, C₁-C₆ alkoxy and —CH₂-(4-6 membered heterocycle). In an embodiment, R⁵ is C₁-C₄ alkyl. In an embodiment, R is selected from -Me, -Et and -iPr. In an embodiment, R⁵ is -Me.

As generally defined herein, each R⁶ is independently selected from C₁-C₄ alkyl, C₁-C₆ alkoxy and —CH₂-(4-6 membered heterocycle). In an embodiment, R⁶ is independently selected from -Me, -Et, —Pr, —ⁱPr, —CH₂CH₂OMe and

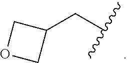 [SMILES: *CC1COC1]

In an embodiment, R⁶ is independently selected from -Me, —CH₂CH₂OMe and

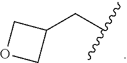 [SMILES: *CC1COC1]

In an embodiment, R⁶ is -Me. In an embodiment, R⁶ is —CH₂CH₂OMe. In an embodiment, R⁶ is

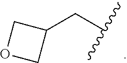 [SMILES: *CC1COC1]

As generally defined herein, R^{d} is H, —F or —OH. In an embodiment, R is selected from the group consisting of H and —F. In an embodiment, R^{d} is —OH. In an embodiment, R^{d} is H. In an embodiment, R^{d} is F.

As generally defined herein, R^{e} is selected from the group consisting of R, R^{e2} or R^{e3}, wherein R^{e1}, R^{e2} and R^{e3} are as defined in any of the embodiments described herein. In an embodiment, R^{e} is selected from R^{e1} and R^{e2} wherein R^{e1} and R^{e2} are as defined in any of the embodiments described herein. In an embodiment, R^{e} is selected from R^{e1} and R^{e3} wherein R^{e1} and R^{e3} are as defined in any of the embodiments described herein. In an embodiment, R^{e} is selected from R^{e2} and R^{e3} wherein R^{e2} and R^{e3} are as defined in any of the embodiments described herein.

In an embodiment, R^{e} is R^{e1} wherein R^{e1} is as defined in any of the embodiments described herein. In an embodiment, R^{e} is R^{e2} wherein R^{e2} is as defined in any of the embodiments described herein. In an embodiment, R is R^{e3} wherein R^{e3} is as defined in any of the embodiments described herein.

As generally defined herein, R^{e1} is a 4-10 membered heterocycle which is substituted with 0, 1, 2, 3 or 4 substituents independently selected from halo, hydroxy, C₁-C₄ alkyl, C₁-C₆ aminoalkyl, 4-6 membered heterocycle, —C(O)C₁-C₆ alkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is a 4-7 membered monocyclic heterocycle containing a nitrogen or an oxygen atom as the only heteroatom, wherein the monocyclic heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is a 4-7 membered monocyclic heterocycle containing a nitrogen atom as the only heteroatom or containing one nitrogen atom and one oxygen atom, wherein the monocyclic heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, the heterocycle of R^{W} is unsubstituted. In an embodiment, the heterocycle of R^{e1} is substituted with 1 substituent as described above. In an embodiment, the heterocycle of R^{e1} is substituted with 2 substituents as described above. In an embodiment, the heterocycle of R^{e1} is substituted with 3 substituents as described above. In an embodiment, the heterocycle of R^{e1} is substituted with 4 substituents as described above.

In an embodiment, the monocyclic heterocycle of R^{e1} is substituted with 0 or 1 instances of C₁-C₄ alkyl. In an embodiment, the monocyclic heterocycle of R^{e1} is substituted with 0 or 1 instances of -Me, -ⁱPr or cyclopropyl. In an embodiment, the monocyclic heterocycle of R^{e1} is substituted with 0 or 1 instances of —ⁱPr. In an embodiment, the monocyclic heterocycle of R^{e1} is substituted with 0 or 1 instances of cyclopropyl. In an embodiment, the monocyclic heterocycle of R^{e1} is substituted with 0 or 1 instance of methyl. In an embodiment, R^{e1} is selected from the group consisting of azetidinyl and oxetanyl, each substituted with 0 or 1 instances of C₁-C₄ alkyl.

In an embodiment, R^{e1} is selected from the group consisting of azetidinyl and oxetanyl, each substituted with 0 or 1 substituents independently selected from -Me, -ⁱPr or cyclopropyl. In an embodiment, R^{e1} is selected from the group consisting of azetidinyl and oxetanyl, each substituted with 0 or 1 instances of-Me. In an embodiment, R^{e1} is selected from the group consisting of azetidinyl and oxetanyl, each substituted with 1 instance of -Me. In an embodiment, R^{e1} is selected from azetidinyl, pyrrolidinyl and morpholinyl substituted with 0 or 1 instance of-Me.

In an embodiment, R^{e1} is azetidinyl substituted with 0 or 1 instances of C₁-C₄ alkyl.

In an embodiment, R^{e1} is azetidinyl substituted with 0 or 1 substituents independently selected from -Me, -ⁱPr or cyclopropyl. In an embodiment, R^{e1} is azetidinyl substituted with 0 or 1 instance of-Me. In an embodiment, R^{e1} is azetidinyl substituted with 0 or 1 instance of cyclopropyl. In an embodiment, R^{e1} is azetidinyl substituted with 0 or 1 instance of —Pr. In an embodiment, R^{e1} is N-methyl azetidinyl.

In an embodiment, R^{e1} is oxetanyl substituted with 0 or 1 instances of C₁-C₄ alkyl. In an embodiment, R^{e1} is oxetanyl substituted with 0 or 1 instance of-Me.

In an embodiment, the attachment point for the monocyclic heterocycle is on a carbon atom.

In an embodiment, R^{e1} is selected from the group consisting of:

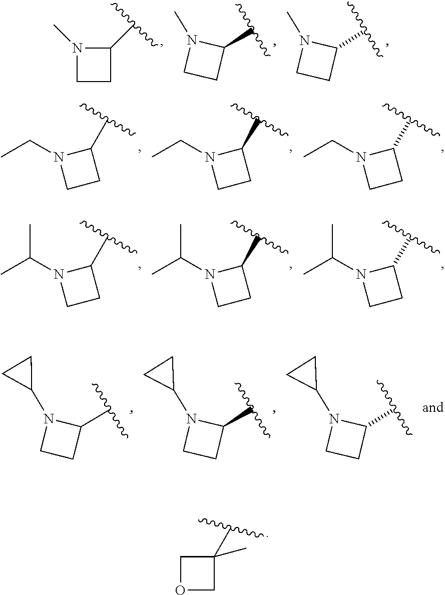

In an embodiment, R^{e1} is selected from the group consisting of:

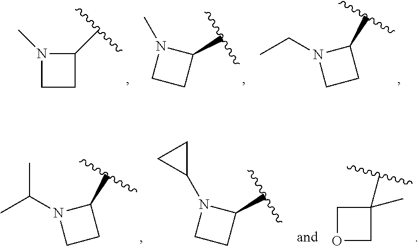

In an embodiment, R^{e1} is selected from the group consisting of:

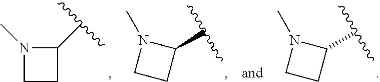

In an embodiment, R^{e1} is

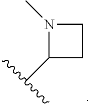 [SMILES: *C1CCN1C]

In an embodiment, R^{e1} is

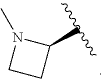 [SMILES: *[C@H]1CCN1C]

In an embodiment, R^{e1} is

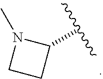 [SMILES: *[C@@H]1CCN1C]

In an embodiment, R^{e1} is

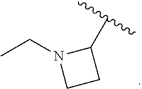 [SMILES: *C1CCN1CC]

In an embodiment, R^{e1} is

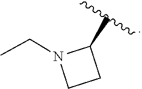 [SMILES: *[C@H]1CCN1CC]

In an embodiment, R^{e1} is

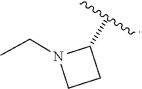 [SMILES: *[C@@H]1CCN1CC]

In an embodiment, R^{e1} is

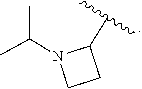 [SMILES: *C1CCN1C(C)C]

In an embodiment, R^{e1} is

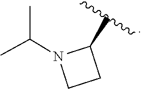 [SMILES: *[C@H]1CCN1C(C)C]

In an embodiment, R^{e1} is

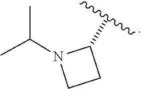 [SMILES: *[C@@H]1CCN1C(C)C]

In an embodiment, R^{e1} is

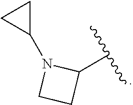 [SMILES: *C1CCN1C1CC1]

In an embodiment, R^{e1} is

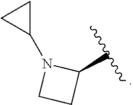 [SMILES: *[C@H]1CCN1C1CC1]

In an embodiment, R^{e1} is

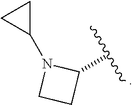 [SMILES: *[C@@H]1CCN1C1CC1]

In an embodiment, R^{e1} is

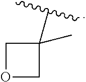 [SMILES: *C1(C)COC1]

In an embodiment, R^{e1} is a 4-10 membered heterocycle containing a nitrogen atom and one or two additional heteroatoms selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is a 4-10 membered heterocycle containing a nitrogen atom and one or two additional heteroatoms selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is a 4-10 membered heterocycle containing a nitrogen atom and one or two additional heteroatoms selected from oxygen and sulfur, including sulfur dioxide, selected from the group consisting of a 4-8 member monocyclic heterocycle, a 6-10 member fused bicyclic heterocycle, a 6-10 member bridged heterocycle and a 6-10 member spiro heterocycle, each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is a 4-10 membered heterocycle containing a nitrogen atom and one or two additional heteroatoms selected from oxygen and sulfur, including sulfur dioxide, selected from the group consisting of a 4-8 member monocyclic heterocycle, a 6-10 member fused bicyclic heterocycle, a 6-10 member bridged heterocycle and a 6-10 member spiro heterocycle, each substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R is a 4-8 member monocyclic heterocycle substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is a 4-8 member monocyclic heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is a 6-10 member fused bicyclic heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is a 6-10 member bridged heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is a 6-10 member spiro heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is selected from azetidine, pyrrolidine, isothiazolidine 1,1-dioxide, 3-azabicyclo[3.1.0]hexane, piperidine, piperazine, 2-oxabicyclo[4.1.0]heptane, 3-oxa-6-azabicyclo[3.2.0]heptane, hexahydro-1H-furo[3,4-c]pyrrole, hexahydro-1H-furo[3,4-b]pyrrole, 2-azabicyclo[2.1.1]hexane, morpholine, 2-oxa-5-azabicyclo[4.1.0]heptane, 1,4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 7-oxa-2-azabicyclo[3.3.1]nonane, 8-oxa-3-azabicyclo[3.2.1]octane, 9-oxa-3-azabicyclo[3.3.1]nonane, 9-oxa-3-azabicyclo[3.3.1]nonane, 2-oxa-6-azaspiro[3.3]heptane, 3-oxa-6-azabicyclo[3.1.1]heptane, 6-oxa-3-azabicyclo[3.1.1]heptane, 2-oxa-5-azabicyclo[2.2.2]octane, 5,6,7,8-tetrahydro-[1,2,4]triazolo[4,3-a]pyrazine, 5,6,7,8-tetrahydro-[1,2,4]triazolo[1,5-a]pyrazine, 4,5,6,7-tetrahydropyrazolo[1,5-a]pyrazine, 5,6,7,8-tetrahydroimidazo[1,2-a]pyrazine, thiomorpholine, thiomorpholine 1,1-dioxide, 4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is selected from azetidine, pyrrolidine, 2-azabicyclo[2.1.1]hexane, morpholine, 2-oxa-5-azabicyclo[4.1.0]heptane, 1,4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 7-oxa-2-azabicyclo[3.3.1]nonane, 8-oxa-3-azabicyclo[3.2.1]octane, 9-oxa-3-azabicyclo[3.3.1]nonane, 9-oxa-3-azabicyclo[3.3.1]nonane, 2-oxa-6-azaspiro[3.3]heptane, 3-oxa-6-azabicyclo[3.1.1]heptane, 6-oxa-3-azabicyclo[3.1.1]heptane, thiomorpholine, thiomorpholine 1,1-dioxide,4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is selected from azetidine, pyrrolidine, isothiazolidine 1,1-dioxide, 3-azabicyclo[3.1.0]hexane, piperidine, piperazine, 2-oxabicyclo[4.1.0]heptane, 3-oxa-6-azabicyclo[3.2.0]heptane, hexahydro-1H-furo[3,4-c]pyrrole, hexahydro-1H-furo[3,4-b]pyrrole, morpholine, 2-oxa-5-azabicyclo[4.1.0]heptane, 1, 4-oxazepane, 2-oxa-5-azabicyclo[2.2.1]heptane, 3,9-dioxa-7-azabicyclo[3.3.1]nonane, 3-oxa-8-azabicyclo[3.2.1]octane, 8-oxa-3-azabicyclo[3.2.1]octane, 2-oxa-6-azaspiro[3.3]heptane, 3-oxa-6-azabicyclo[3.1.1]heptane, 6-oxa-3-azabicyclo[3.1.1]heptane, 2-oxa-5-azabicyclo[2.2.2]octane, 5,6,7,8-tetrahydro-[1,2,4]triazolo[4,3-a]pyrazine, 5,6,7,8-tetrahydro-[1,2,4]triazolo[1,5-a]pyrazine, 4,5,6,7-tetrahydropyrazolo[1,5-a]pyrazine, 5,6,7,8-tetrahydroimidazo[1,2-a]pyrazine, thiomorpholine and thiomorpholine 1,1-dioxide, each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, the heterocycle of R^{e1} is unsubstituted. In an embodiment, the heterocycle of R^{e1} is substituted with 1 substituent as described above. In an embodiment, the heterocycle of R^{e1} is substituted with 2 substituents as described above. In an embodiment, the heterocycle of R^{e1} is substituted with 3 substituents as described above. In an embodiment, the heterocycle of R^{e1} is substituted with 4 substituents as described above. In an embodiment of any of the embodiments of R^{e1} described above, each of the substituents is independently selected from halo, C₁-C₄ alkyl, 4-6 membered heterocycle, —C(O)C₁-C₆ alkyl, C₁-C₆ alkoxy, and C₁-C₄ haloalkoxy. In an embodiment of any of the embodiments described above, each of the substituents is halo. In an embodiment of any of the embodiments described above, each of the substituents is C₁-C₄ alkyl. In an embodiment of any of the embodiments described above, each of the substituents is a 4-6 membered heterocycle. In an embodiment of any of the embodiments described above, each of the substituents is —C(O)C₁-C₆ alkyl. In an embodiment of any of the embodiments described above, each of the substituents is C₁-C₆ alkoxy. In an embodiment of any of the embodiments described above, each of the substituents is C₁-C₄ haloalkoxy.

In an embodiment of any of the embodiments described above, each of the substituents is independently selected from —F, -Me, —OMe, —OEt, —OCHF₂, —C(═O)Me and oxetanyl. In an embodiment of any of the embodiments described above, each of the substituents is —F. In an embodiment of any of the embodiments described above, each of the substituents is -Me. In an embodiment of any of the embodiments described above, each of the substituents is —OMe. In an embodiment of any of the embodiments described above, each of the substituents is —OEt. In an embodiment of any of the embodiments described above, each of the substituents is —OCHF₂. In an embodiment of any of the embodiments described above, each of the substituents is —C(═O)Me. In an embodiment of any of the embodiments described above, each of the substituents is oxetanyl.

In an embodiment, R^{e1} is selected from azetidine, pyrrolidine, isothiazolidine 1,1-dioxide, 3-azabicyclo[3.1.0]hexane, piperidine, piperazine, 2-oxabicyclo[4.1.0]heptane, 3-oxa-6-azabicyclo[3.2.0]heptane, hexahydro-1H-furo[3,4-c]pyrrole, hexahydro-1H-furo[3,4-b]pyrrole, morpholine, 2-oxa-5-azabicyclo[4.1.0]heptane, 1, 4-oxazepane, 2-oxa-5-azabicyclo[2.2.1]heptane, 3,9-dioxa-7-azabicyclo[3.3.1]nonane, 3-oxa-8-azabicyclo[3.2.1]octane, 8-oxa-3-azabicyclo[3.2.1]octane, 2-oxa-6-azaspiro[3.3]heptane, 3-oxa-6-azabicyclo[3.1.1]heptane, 6-oxa-3-azabicyclo[3.1.1]heptane, 2-oxa-5-azabicyclo[2.2.2]octane, 5,6,7,8-tetrahydro-[1,2,4]triazolo[4,3-a]pyrazine, 5,6,7,8-tetrahydro-[1,2,4]triazolo[1,5-a]pyrazine, 4,5,6,7-tetrahydropyrazolo[1,5-a]pyrazine, 5,6,7,8-tetrahydroimidazo[1,2-a]pyrazine, thiomorpholine and thiomorpholine 1,1-dioxide, each substituted with 0, 1 or 2 substituents independently selected from —F, -Me, —OMe, —OEt, —OCHF₂, —C(═O)Me and oxetanyl.

In an embodiment, R^{e1} is selected from azetidine, pyrrolidine, piperidine, hexahydro-1H-furo[3,4-c]pyrrole, morpholine, 1, 4-oxazepane, 2-oxa-5-azabicyclo[2.2.1]heptane, 3-oxa-8-azabicyclo[3.2.1]octane, 8-oxa-3-azabicyclo[3.2.1]octane, 3-oxa-6-azabicyclo[3.1.1]heptane, 6-oxa-3-azabicyclo[3.1.1]heptane, 2-oxa-5-azabicyclo[2.2.2]octane and thiomorpholine, each substituted with 0, 1 or 2 substituents independently selected from —F, -Me, —OMe and -OEt.

In an embodiment, R^{e1} is selected from hexahydro-1H-furo[3,4-c]pyrrole, morpholine, 1, 4-oxazepane, 2-oxa-5-azabicyclo[2.2.1]heptane, 3-oxa-8-azabicyclo[3.2.1]octane, 8-oxa-3-azabicyclo[3.2.1]octane, 3-oxa-6-azabicyclo[3.1.1]heptane, 6-oxa-3-azabicyclo[3.1.1]heptane, 2-oxa-5-azabicyclo[2.2.2]octane and thiomorpholine, each unsubstituted.

In an embodiment, R^{e1} is azetidine substituted with 0, 1 or 2 substituents independently selected from —F and —OMe and -OEt.

In an embodiment, R^{e1} is pyrrolidine substituted with 0, 1 or 2 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is pyrrolidine substituted with 0, 1 or 2 substituents independently selected from —F, —OMe, —OEt and —OCHF₂.

In an embodiment, R^{e1} is piperidine substituted with 0, 1 or 2 instances of—F. In an embodiment, R^{e1} is unsubstituted hexahydro-1H-furo[3,4-c]pyrrole. In an embodiment, R^{e1} is unsubstituted 1, 4-oxazepane. In an embodiment, R^{e1} is unsubstituted 2-oxa-5-azabicyclo[2.2.1]heptane. In an embodiment, R^{e1} is unsubstituted 3-oxa-8-azabicyclo[3.2.1]octane. In an embodiment, R^{e1} is unsubstituted 8-oxa-3-azabicyclo[3.2.1]octane. In an embodiment, R^{e1} is unsubstituted 3-oxa-6-azabicyclo[3.1.1]heptane. In an embodiment, R^{e1} is unsubstituted 6-oxa-3-azabicyclo[3.1.1]heptane. In an embodiment, R^{e1} is unsubstituted 2-oxa-5-azabicyclo[2.2.2]octane. In an embodiment, R^{e1} is unsubstituted thiomorpholine.

In an embodiment, R^{e1} is morpholine substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl. In an embodiment, R^{e1} is morpholine substituted with 0, 1 or 2 instances of -Me.

In an embodiment, the attachment point for R is the nitrogen atom of the heterocycle.

In an embodiment, the R is selected from the group consisting of:

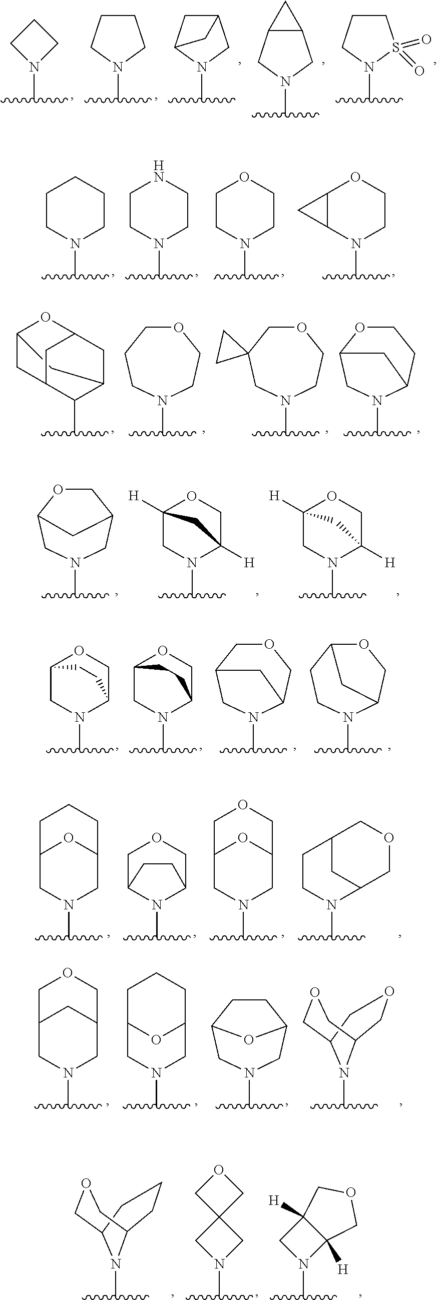

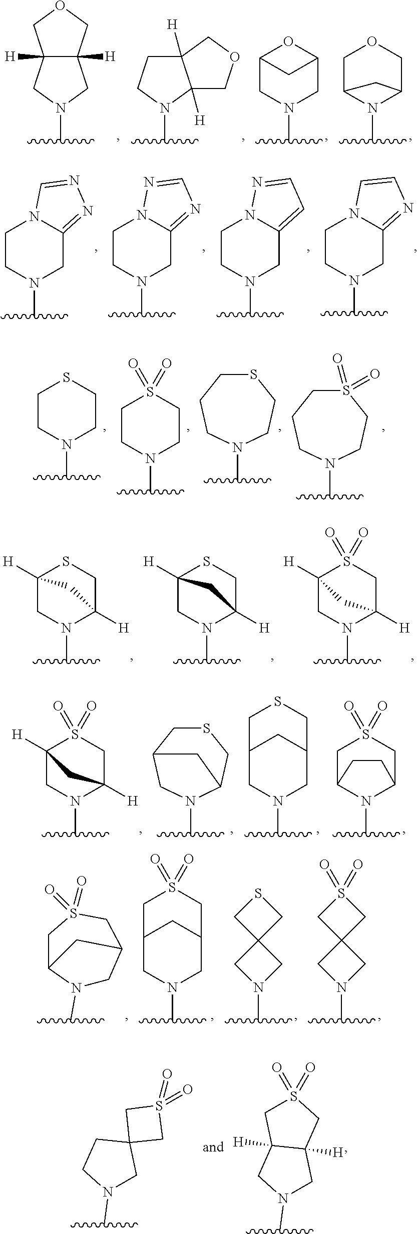

each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, the R^{d} is selected from the group consisting of:

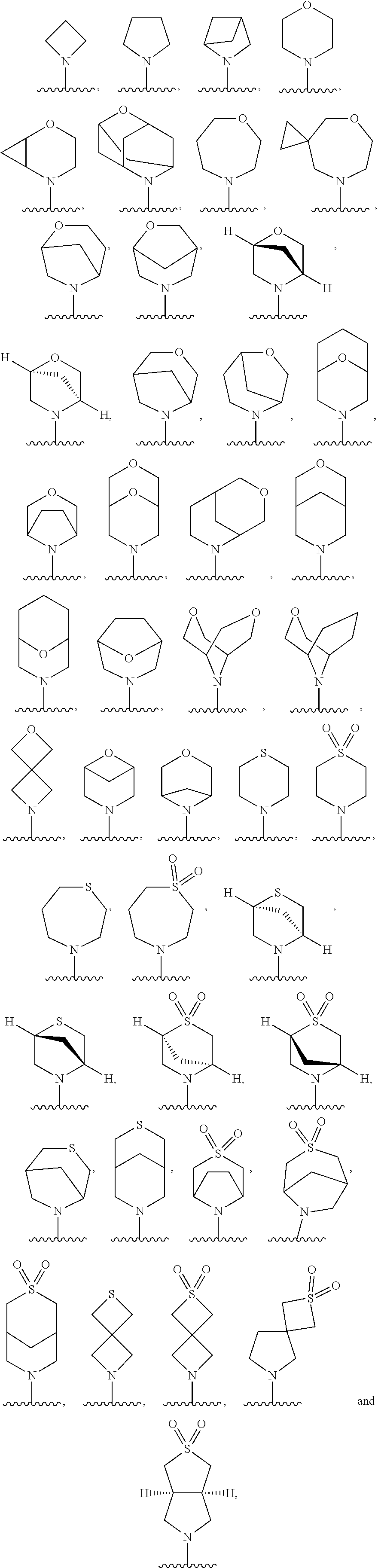

each substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, the R is selected from the group consisting of:

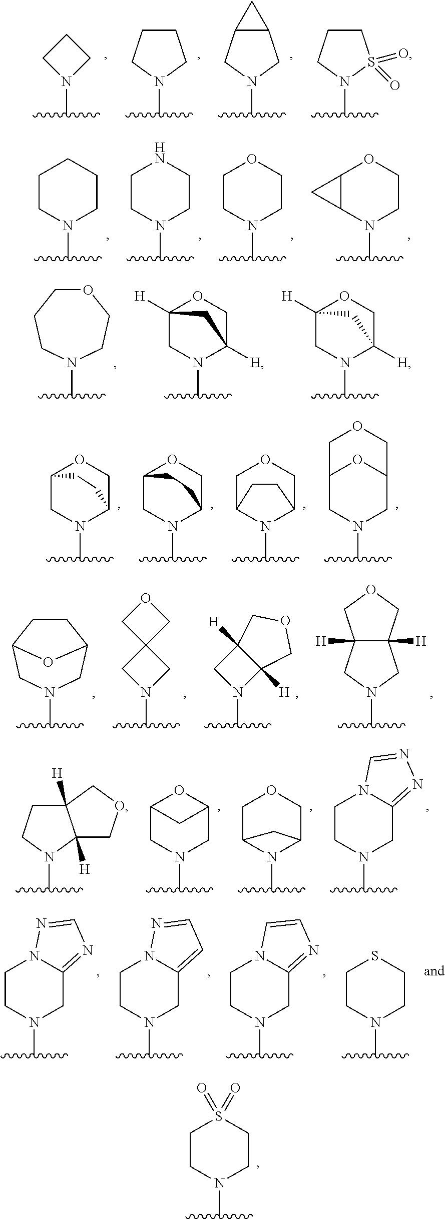

each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is

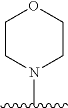 [SMILES: *N1CCOCC1]

substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, the 4-10 membered heterocycle of R^{e1} is substituted with 0, 1, 2, 3 or 4 substituents independently selected from —F, -Me, -Et, —OH, —OMe, —CF₃, —OCF₃, —CH₂OCH₃, —CH₂CH₂OCH₃, —CH₂CH₂OCH₂CH₂OCH₃, —CH₂N(CH₃)₂, —CH₂CH₂N(CH₃)₂.

In an embodiment, the 4-10 membered heterocycle of R^{e1} is substituted with 0, 1 or 2 substituents independently selected from —F, -Me, —OMe, —OEt, —OCHF₂, —C(═O)Me and oxetanyl.

In an embodiment, the 4-10 membered heterocycle of R^{e} is substituted with 0, 1 or 2 substituents independently selected from fluoro, methyl, ethyl, hydroxy, methoxy, trifluoromethyl, trifluoromethoxy, —CH₂OCH₃, —CH₂CH₂OCH₃, —CH₂CH₂OCH₂CH₂OCH₃, —CH₂N(CH₃)₂, —CH₂CH₂N(CH₃)₂.

In an embodiment, the 4-10 membered heterocycle of R^{e1} is substituted with 0, 1 or 2 substituents independently selected from fluoro and methyl.

In an embodiment, R^{e1} is selected from

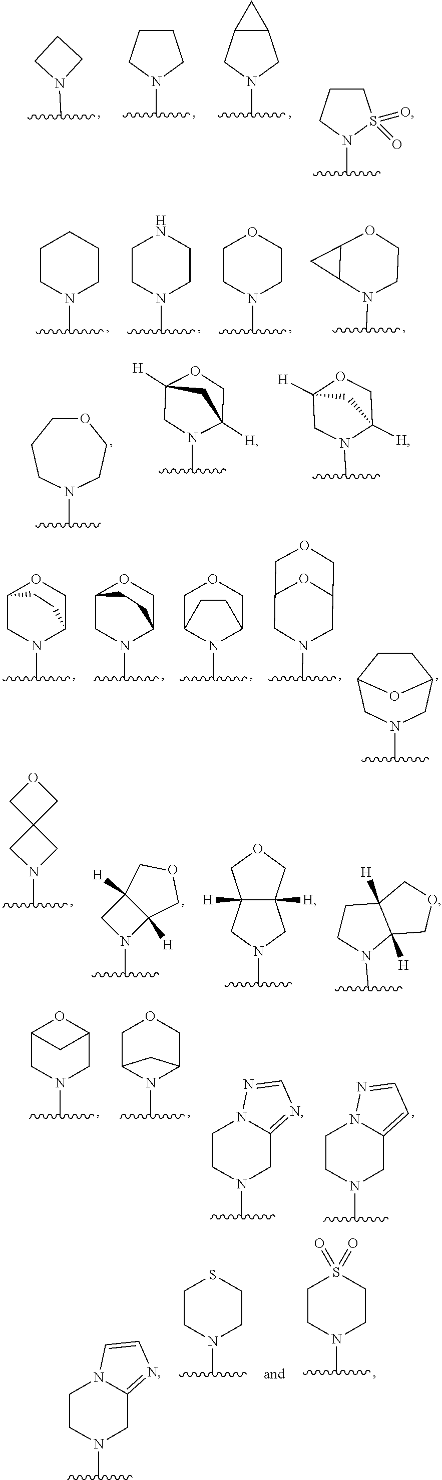

each substituted with 0, 1 or 2 substituents independently selected from —F, -Me, —OMe, —OEt, —OCHF₂, —C(═O)Me and oxetanyl.

In an embodiment, R is selected from

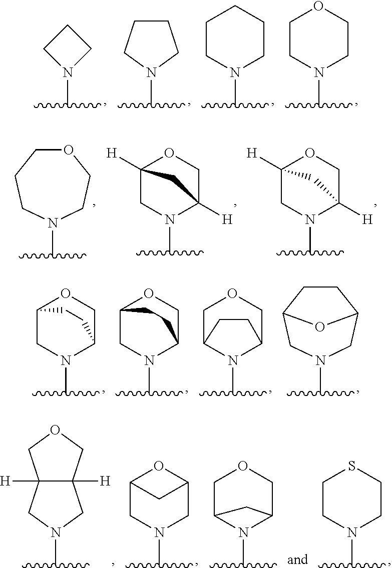

and each substituted with 0, 1 or 2 substituents independently selected from —F, -Me, —OMe and -OEt.

In an embodiment, the 4-10 membered heterocycle of R^{e1} is unsubstituted.

In an embodiment, R^{e1} is selected from,

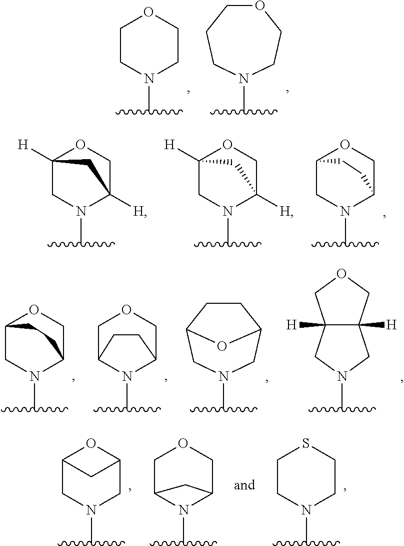

and each unsubstituted.

In an embodiment, R^{e1} is

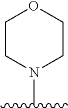 [SMILES: *N1CCOCC1]

substituted with 0, 1 or 2 instances of -Me.

In an embodiment, R^{e1} is

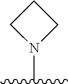 [SMILES: *N1CCC1]

substituted with 0, 1 or 2 substituents independently selected from —F and —OMe and -OEt.

In an embodiment, R^{e1} is

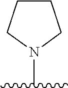 [SMILES: *N1CCCC1]

substituted with 0, 1 or 2 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

In an embodiment, R^{e1} is

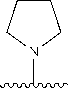 [SMILES: *N1CCCC1]

substituted with 0, 1 or 2 substituents independently selected from —F, —OMe, —OEt and —OCHF₂.

In an embodiment, R^{e1} is

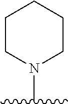 [SMILES: *N1CCCCC1]

substituted with 0, 1 or 2 instances of —F.

In an embodiment, R^{e1} is selected from the group consisting of:

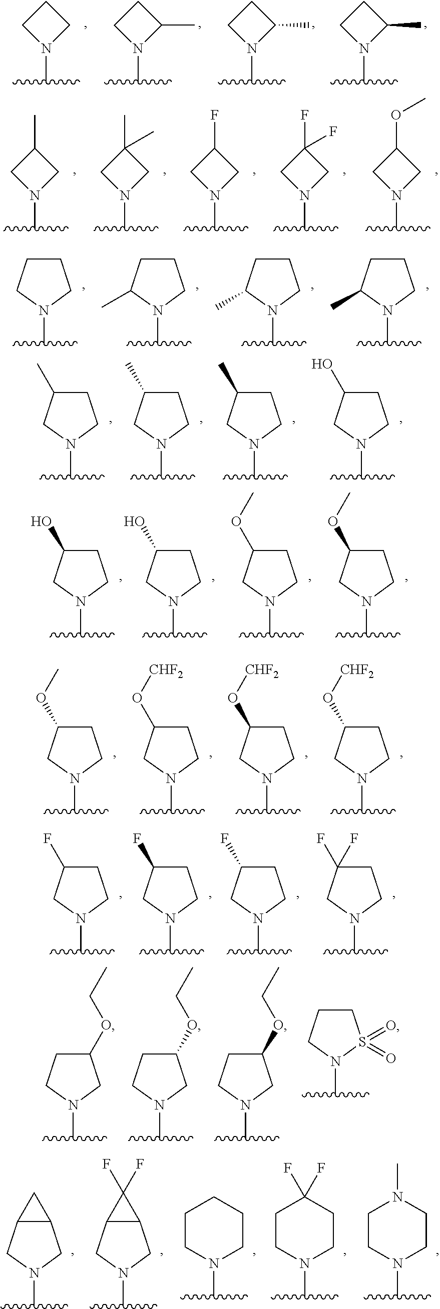

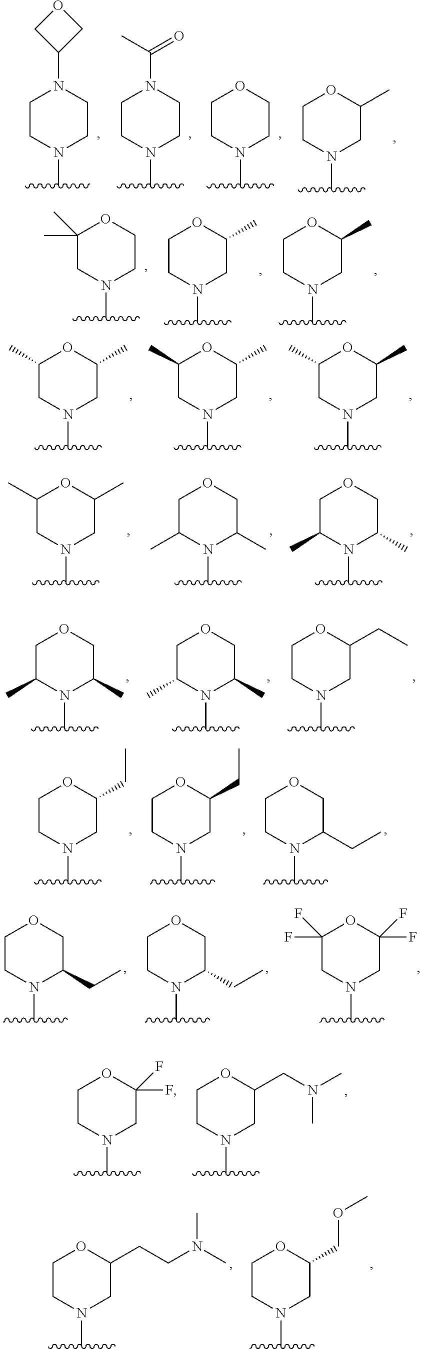

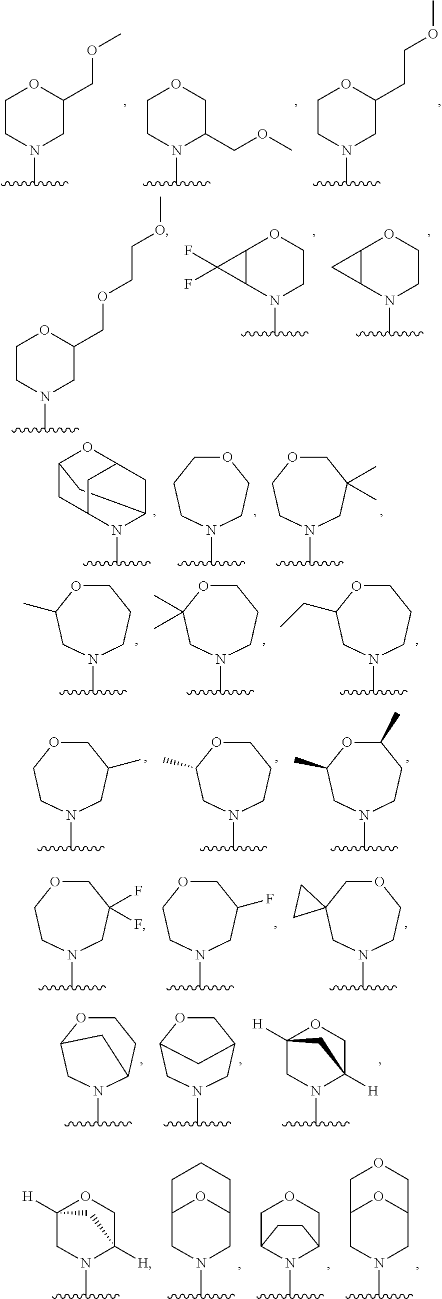

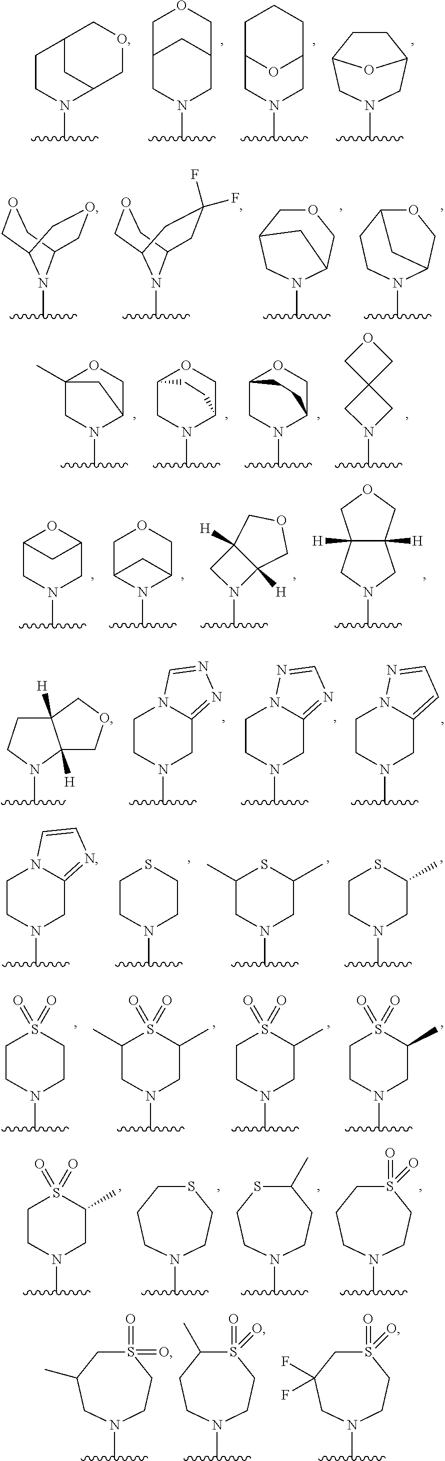

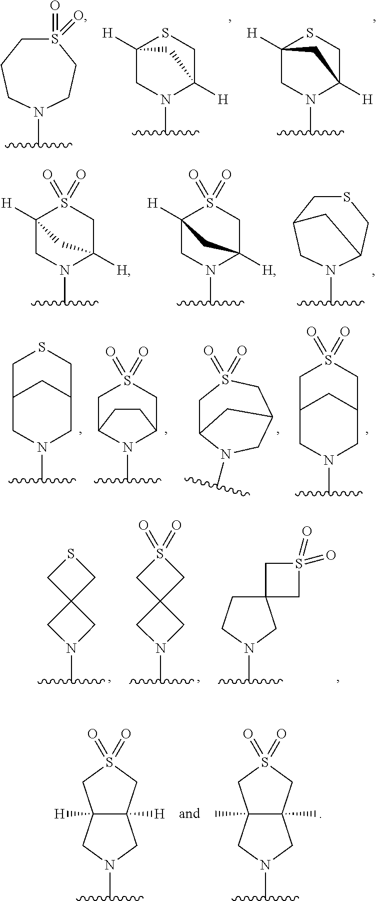

In an embodiment, R^{e1} is selected from the group consisting of:

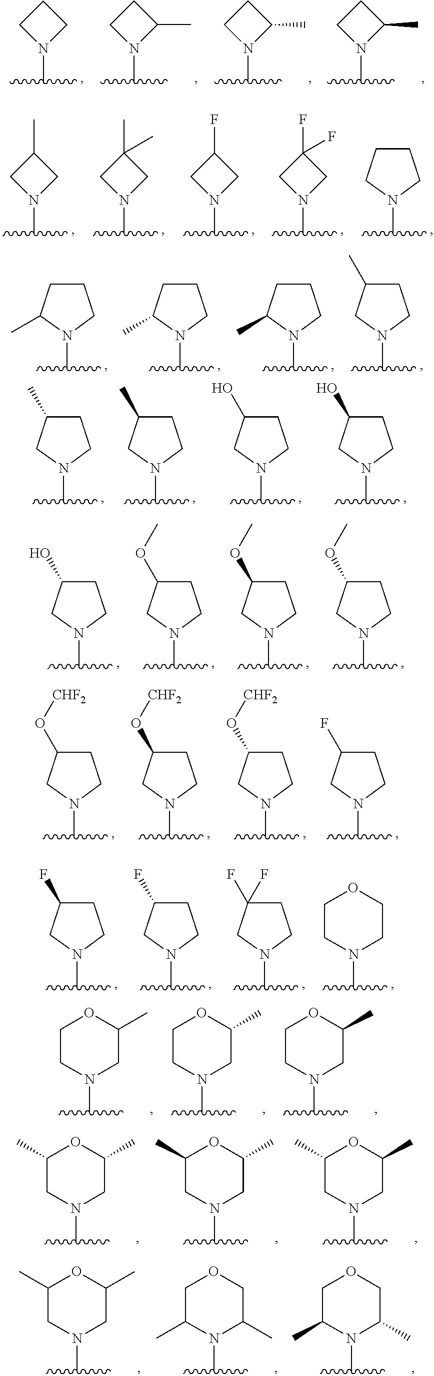

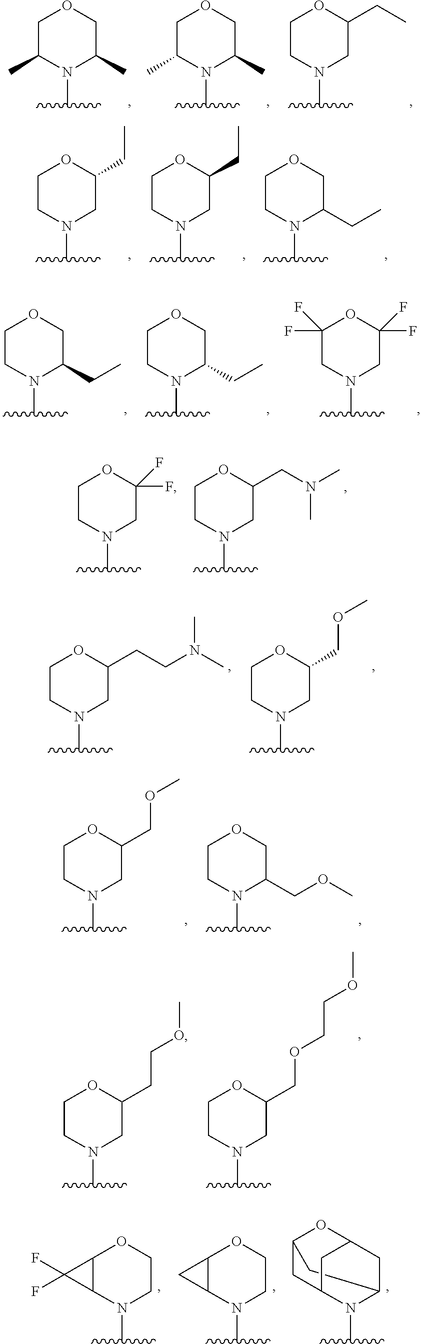

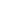

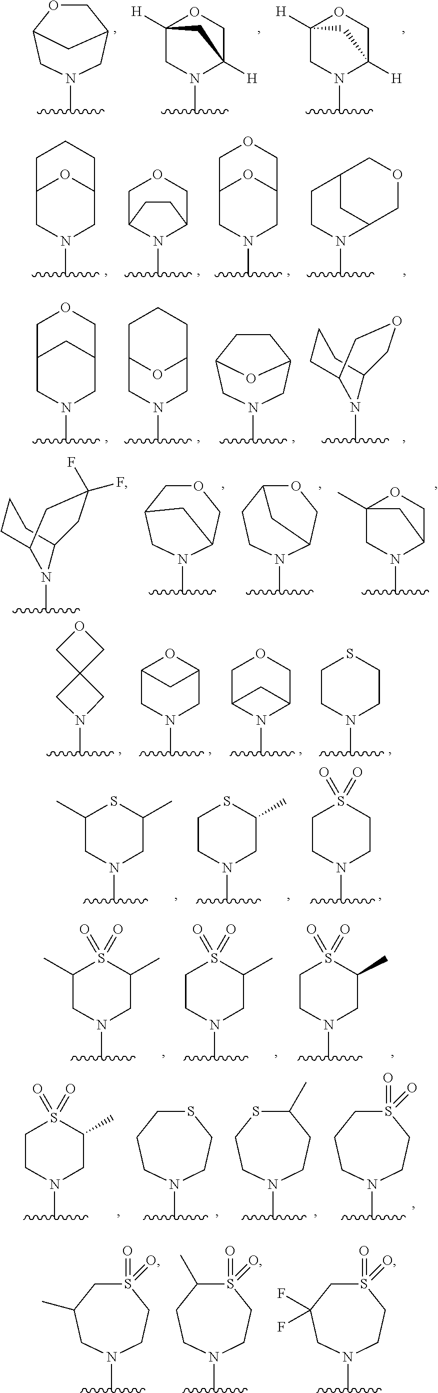

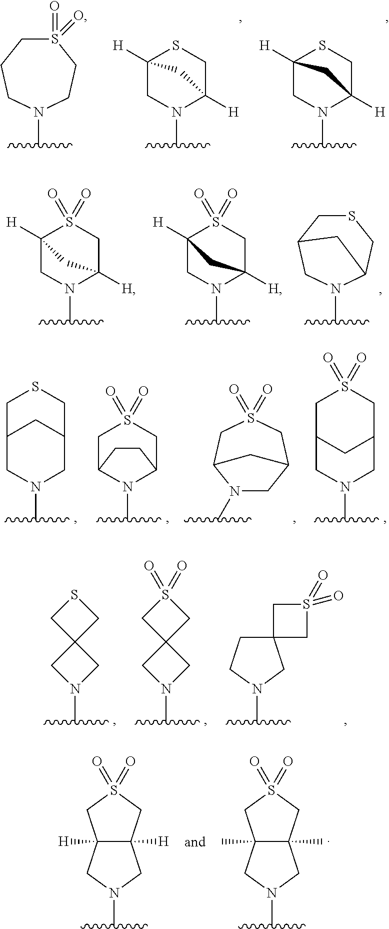

In an embodiment, R^{e1} is selected from the group consisting of

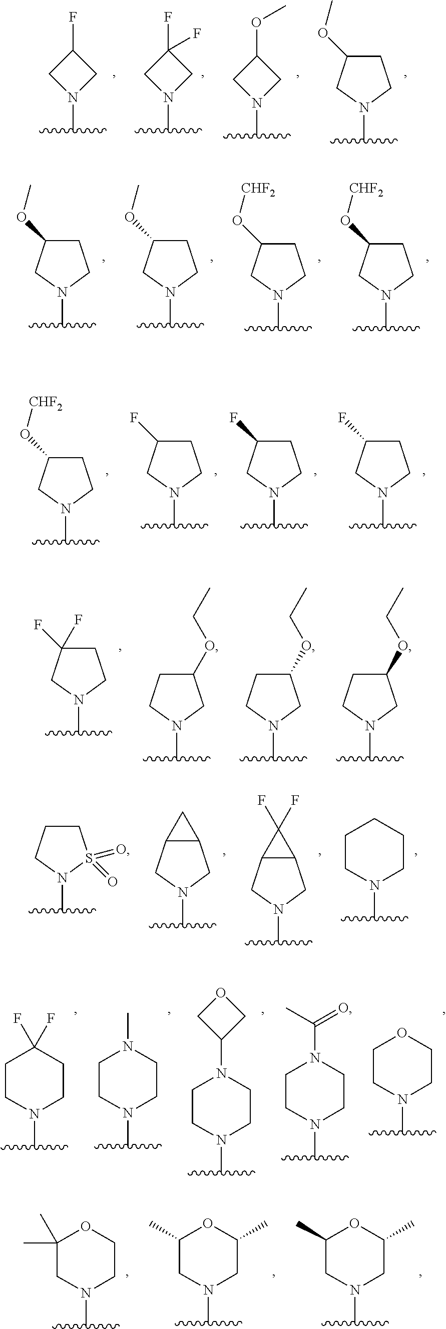

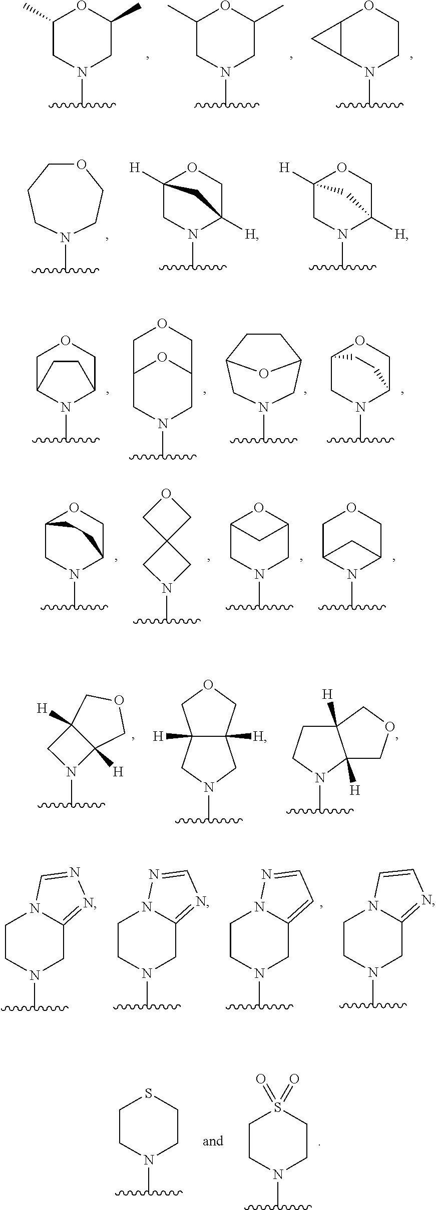

In an embodiment, R^{e1} is selected from the group consisting of:

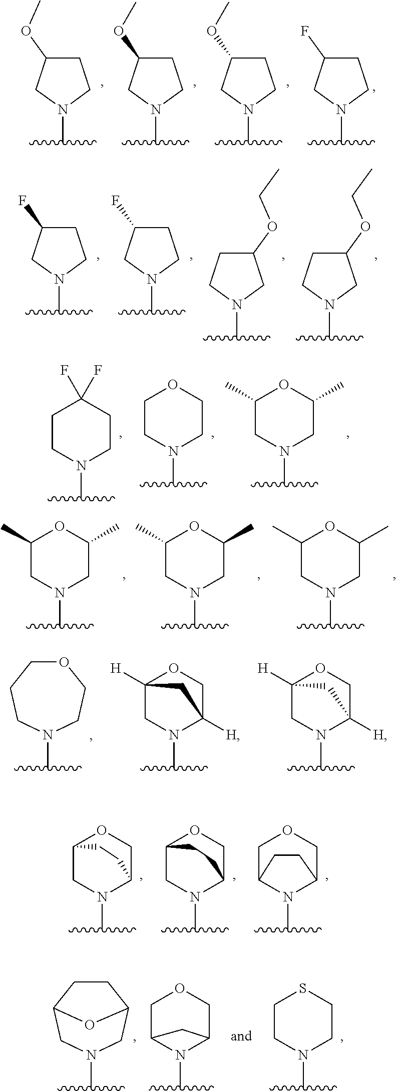

In an embodiment, R^{e1} is selected from the group consisting of:

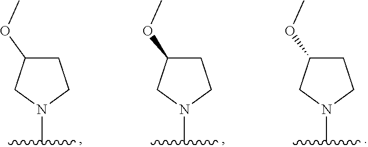

In an embodiment, R^{e1} is selected from the group consisting of:

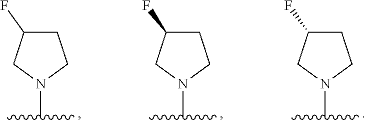 [SMILES: *N1CCC(F)C1 ; *N1CC[C@H](F)C1 ; *N1CC[C@@H](F)C1]

In an embodiment, R^{e1} is selected from the group consisting of:

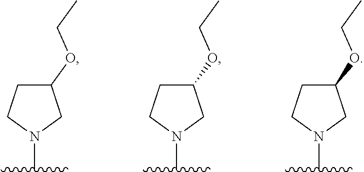

In an embodiment, R^{e1} is

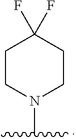 [SMILES: *N1CCC(F)(F)CC1]

In an embodiment, R^{e1} is selected from the group consisting of.

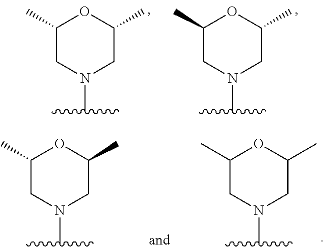

In an embodiment, R^{e1} is unsubstituted

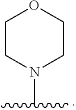 [SMILES: *N1CCOCC1]

In an embodiment, R^{e1} is unsubstituted

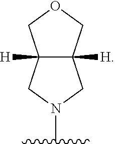

In an embodiment, R^{e1} is unsubstituted

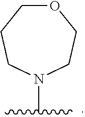 [SMILES: *N1CCCOCC1]

In an embodiment, R^{e1} is unsubstituted

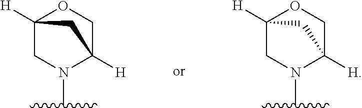

In an embodiment, R^{e1} is unsubstituted

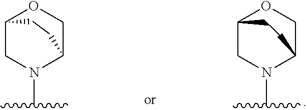 [SMILES: *N1C[C@@H]2CC[C@H]1CO2 ; "or" ; *N1C[C@H]2CC[C@@H]1CO2]

In an embodiment, R^{e1} is unsubstituted

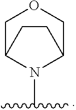 [SMILES: *N1C2CCC1COC2]

In an embodiment, R^{e1} is unsubstituted

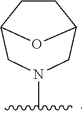 [SMILES: *N1CC2CCC(C1)O2]

In an embodiment, R^{e1} is unsubstituted

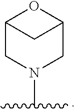 [SMILES: *N1CC2CC(C1)O2]

In an embodiment, R^{e1} is unsubstituted

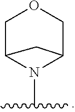 [SMILES: *N1C2COCC1C2]

In an embodiment, R^{e1} is unsubstituted.

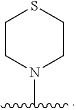 [SMILES: *N1CCSCC1]

As generally defined herein, R^{e2} is a 5-6 membered heteroaryl wherein the attachment point is a carbon atom group and wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is a 5-6 membered heteroaryl group containing at least one nitrogen atom, wherein the attachment point for the heteroaryl group is a carbon atom group and wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is selected from the group consisting of pyrimidinyl, pyrazinyl, oxazolyl, 1,2,4-oxadiazolyl, 1,3,4-oxadiazolyl, 1H-1,2,4-triazolyl, imidazolyl, 4H-1,2,4-triazolyl, 1H-1,2,4-triazolyl, 2-H-tetrazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is selected from the group consisting of pyrimidinyl, pyrazinyl, oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is selected from the group consisting of pyrimidinyl, oxazolyl, 1,2,4-oxadiazolyl and 1,2,4-thiadiazolyl each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is selected from the group consisting of pyrimidinyl and 1,2,4-oxadiazolyl each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is selected from the group consisting of

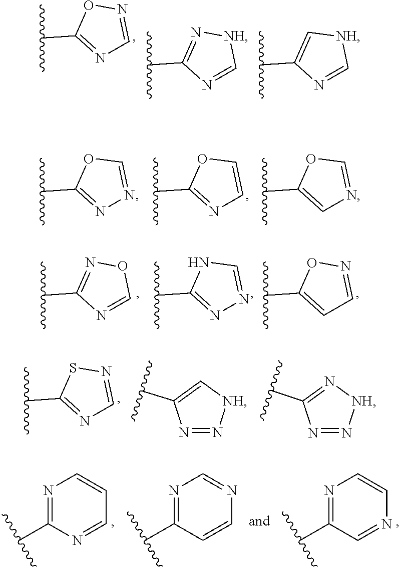

each substituted with 0, 1 or 2 substituents independently selected from halo, hydroxy, C₁-C₄ acyclic alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment R^{e2} is selected from the group consisting of

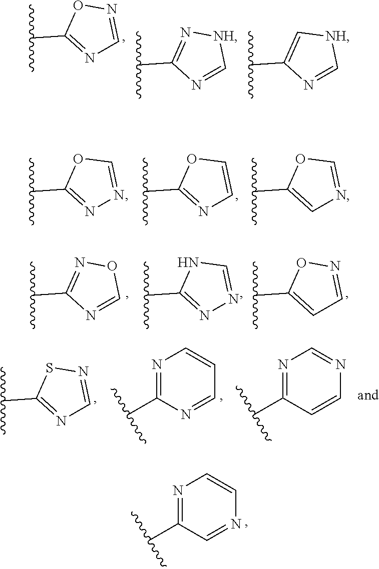

each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is selected from the group consisting of

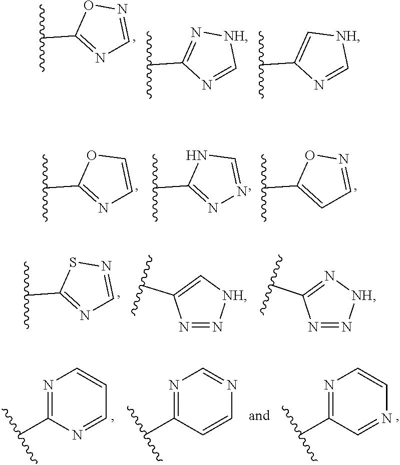

each substituted with 0, 1 or 2 substituents independently selected from halo, hydroxy, C₁-C₄ acyclic alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is selected from the group consisting of

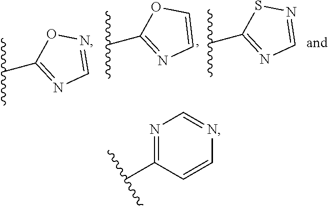

each substituted with 0, 1 or 2 substituents independently selected from halo, C₁-C₄ acyclic alkyl, C₁-C₄ hydroxyalkyl, C₁-C₄ haloalkyl, C₃-C₆ heterocyclyl and C₃-C₆ cycloalkyl optionally substituted with one or two instances of halo or methyl.

In an embodiment, R^{e2} is selected from the group consisting of

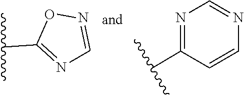 [SMILES: *c1ncno1 ; *c1ccncn1]

each substituted with 0, 1 or 2 substituents independently selected from halo, C₁-C₄ acyclic alkyl, C₁-C₄ hydroxyalkyl, C₁-C₄ haloalkyl, C₃-C₆ heterocyclyl and C₃-C₆ cycloalkyl optionally substituted with one or two instances of halo or methyl.

In an embodiment, R^{e2} is selected from the group consisting of

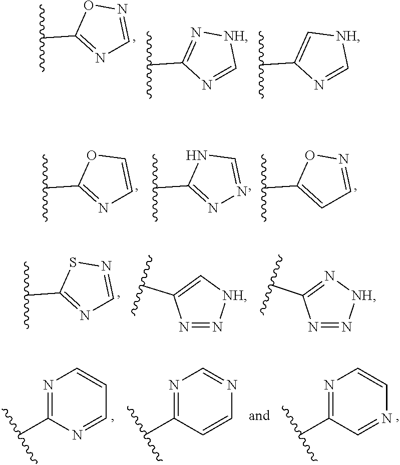

each substituted with 0, 1 or 2 substituents independently selected from —F, -Me, -Et, -iPr, -tBu, —C(OH)(CH₃)₂, —CHF₂, —CF₂CH₃, —CH(F)CH₃, —CF₃, oxetanyl, cyclopropyl, 1-Me-cyclopropyl and 2-F-cyclopropyl.

In an embodiment, R^{e2} is selected from the group consisting of

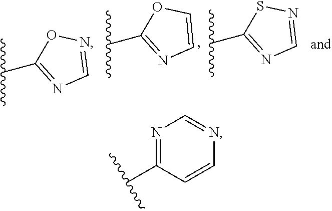

each substituted with 0, 1 or 2 substituents independently selected from —F, -Me, -Et, -iPr, -tBu, —C(OH)(CH₃)₂, —CHF₂, —CF₂CH₃, —CH(F)CH₃, —CF₃, oxetanyl, cyclopropyl, 1-Me-cyclopropyl and 2-F-cyclopropyl.

In an embodiment, R^{e2} is selected from the group consisting of

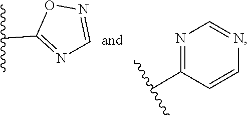 [SMILES: *c1ncno1 ; *c1ccncn1]

each substituted with 0, 1 or 2 substituents independently selected from —F, -Me, -Et, -iPr, -tBu, —C(OH)(CH₃)₂, —CHF₂, —CF₂CH₃, —CH(F)CH₃, —CF₃, oxetanyl, cyclopropyl, 1-Me-cyclopropyl and 2-F-cyclopropyl.

In an embodiment, the heteroaryl of R^{e2} is unsubstituted. In an embodiment, the heteroaryl of R^{e2} is substituted with 1 substituent as described above. In an embodiment, the heteroaryl of R^{e2} is substituted with 2 substituents as described above. In an embodiment of any of the embodiments of R^{e2} described above, each of the substituents is independently selected from halo, C₁-C₄ acyclic alkyl, C₁-C₄ hydroxyalkyl, C₁-C₄ haloalkyl, C₃-C₆ heterocyclyl and C₃-C₆ cycloalkyl optionally substituted with one or two instances of halo or methyl. In an embodiment of any of the embodiments described above, each of the substituents is halo. In an embodiment of any of the embodiments described above, each of the substituents is C₁-C₄ acyclic alkyl. In an embodiment of any of the embodiments described above, each of the substituents is C₁-C₄ hydroxyalkyl. In an embodiment of any of the embodiments described above, each of the substituents is C₁-C₄ haloalkyl. In an embodiment of any of the embodiments described above, each of the substituents is C₃-C₆ heterocyclyl (e.g., containing 1 or 2 heteroatoms selected from N and O). In an embodiment of any of the embodiments described above, each of the substituents is C₃-C₆ cycloalkyl optionally substituted with one or two instances of halo or methyl. In an embodiment of any of the embodiments described above, each of the substituents is independently selected from —F, -Me, -Et, -iPr, -tBu, —C(OH)(CH₃)₂, —CHF₂, —CF₂CH₃, —CH(F)CH₃, —CF₃, oxetanyl, cyclopropyl, 1-Me-cyclopropyl and 2-F-cyclopropyl. In an embodiment of any of the embodiments described above, each of the substituents is —F. In an embodiment of any of the embodiments described above, each of the substituents is -Me. In an embodiment of any of the embodiments described above, each of the substituents is -Et. In an embodiment of any of the embodiments described above, each of the substituents is -iPr. In an embodiment of any of the embodiments described above, each of the substituents is -tBu. In an embodiment of any of the embodiments described above, each of the substituents is —C(OH)(CH₃)₂. In an embodiment of any of the embodiments described above, each of the substituents is oxetanyl. In an embodiment of any of the embodiments described above, each of the substituents is —CHF₂. In an embodiment of any of the embodiments described above, each of the substituents is —CF₂CH₃. In an embodiment of any of the embodiments described above, each of the substituents is —CH(F)CH₃. In an embodiment of any of the embodiments described above, each of the substituents is —CF₃. In an embodiment of any of the embodiments described above, each of the substituents is cyclopropyl. In an embodiment of any of the embodiments described above, each of the substituents is 1-Me-cyclopropyl. In an embodiment of any of the embodiments described above, each of the substituents is 2-F-cyclopropyl.

In an embodiment, R^{e2} is selected from the group consisting of:

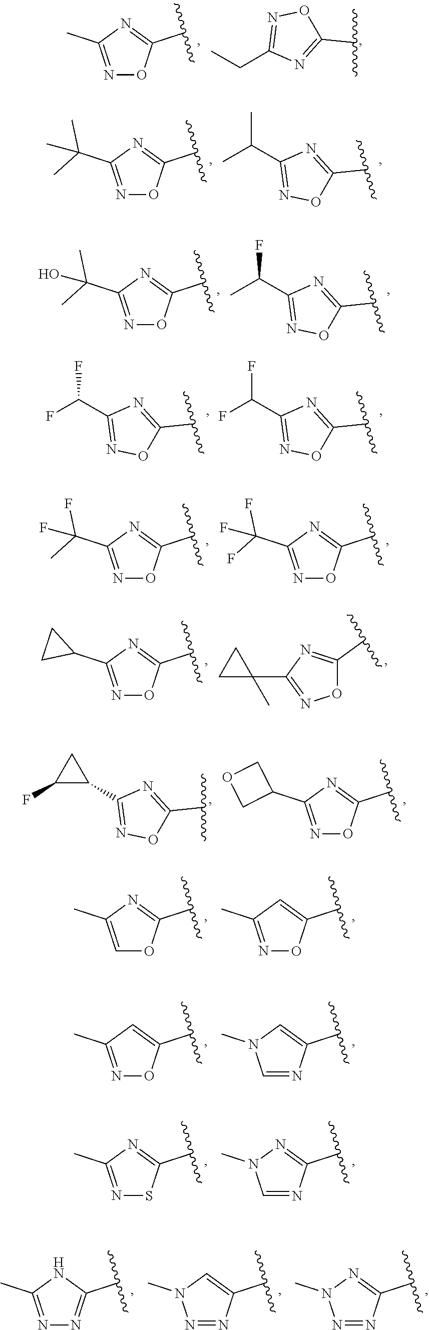

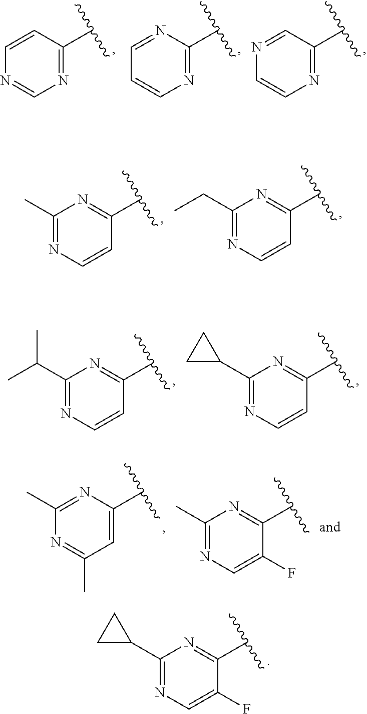

In an embodiment, R^{e2} is selected from the group consisting of:

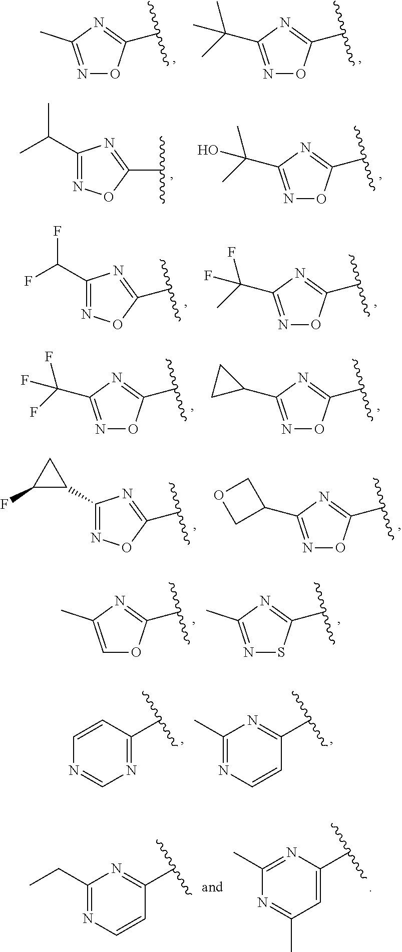

In an embodiment, R^{e2} is selected from the group consisting of:

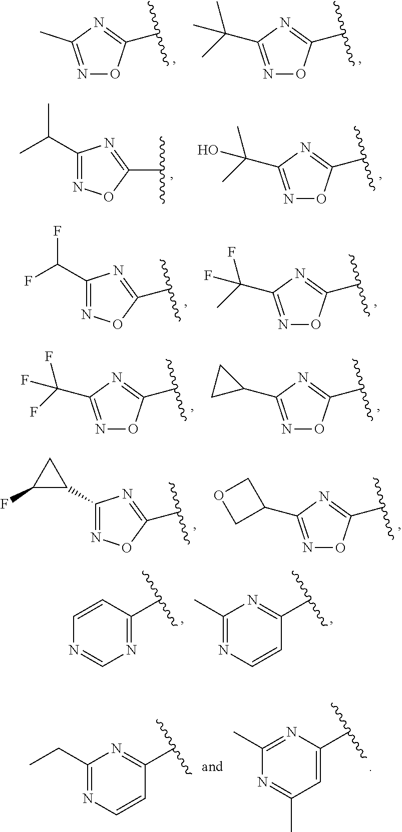

In an embodiment, R^{e2} is a 6 membered heteroaryl group substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is a 6 membered heteroaryl group substituted with 0, 1 or 2 substituents independently selected from halo, acyclic C₁-C₄ alkyl, and C₃-C₆ cycloalkyl.

In an embodiment, R^{e2} is a 6 membered heteroaryl group substituted with 0, 1 or 2 substituents independently selected from —F, -Me, -Et, -iPr, and cyclopropyl.

In an embodiment, R^{e2} is pyrimidinyl or pyridazinyl substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is unsubstituted pyrimidinyl or pyridazinyl.

In an embodiment, R^{e2} is pyrimidinyl substituted with 0, 1 or 2 substituents independently selected from halo, acyclic C₁-C₄ alkyl, and C₃-C₆ cycloalkyl.

In an embodiment, R^{e2} is pyrimidinyl substituted with 0, 1 or 2 substituents independently selected from —F, -Me, -Et, -iPr, and cyclopropyl.

In an embodiment, R^{e2} is selected from the group consisting of

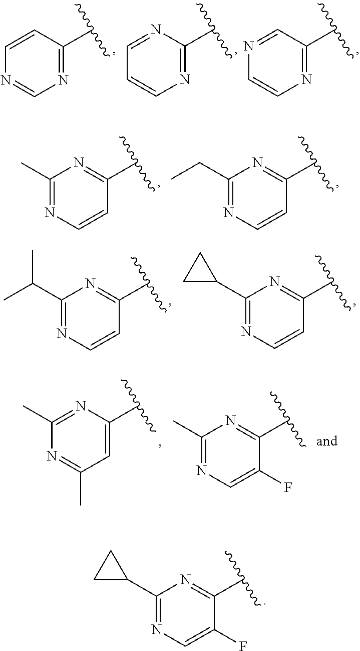

In an embodiment, R^{e2} is selected from the group consisting of

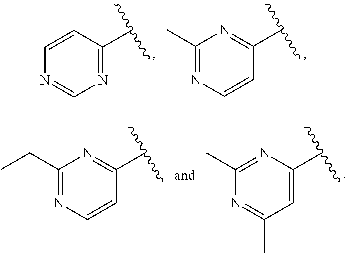

In an embodiment, R^{e2} is selected from the group consisting of

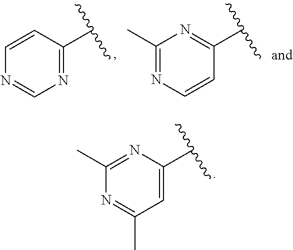

In an embodiment, R^{e2} is

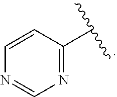 [SMILES: *c1ccncn1]

In an embodiment, R^{e2} is

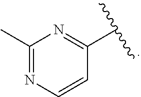 [SMILES: *c1ccnc(C)n1]

In an embodiment, R^{e2} is

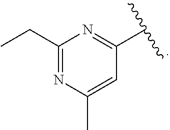 [SMILES: *c1cc(C)nc(CC)n1]

In an embodiment, R^{e2} is

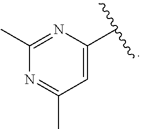 [SMILES: *c1cc(C)nc(C)n1]

In an embodiment, R^{e2} is selected from the group consisting of

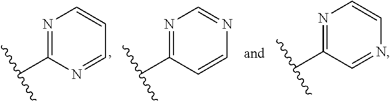

each unsubstituted.

In an embodiment, R^{e2} is a 5 membered heteroaryl group containing at least one nitrogen atom, wherein the attachment point for the heteroaryl group is a carbon atom group and wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is selected from the group consisting of oxazolyl, 1,2,4-oxadiazolyl, 1,3,4-oxadiazolyl, 1H-1,2,4-triazolyl, imidazolyl, 4H-1,2,4-triazolyl, 1H-1,2,4-triazolyl, 2-H-tetrazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is selected from the group consisting of oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is selected from the group consisting of oxazolyl, 1,2,4-oxadiazolyl and 1,2,4-thiadiazolyl each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is selected from the group consisting of

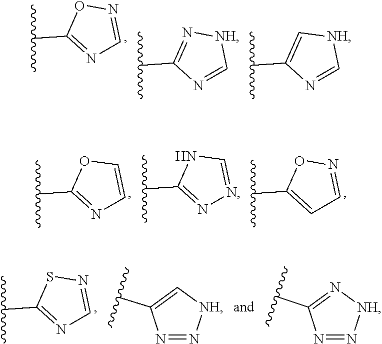

each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is selected from the group consisting of

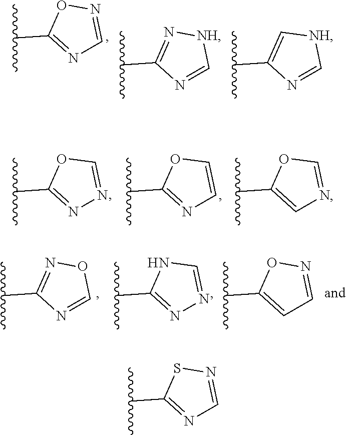

substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is selected from the group consisting of

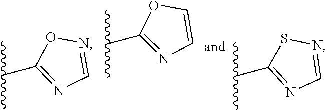

each substituted with 0 or 1 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is

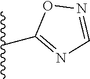 [SMILES: *c1ncno1]

substituted with 0 or 1 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, the 5 membered heteroaryl group of R^{e2} is substituted with 0 or 1 substituents independently selected from acyclic C₁-C₄ alkyl, C₁-C₄ hydroxyalkyl, C₁-C₄ haloalkyl, C₃-C₆ heterocyclyl and C₃-C₆ cycloalkyl optionally substituted with one or two instances of halo or methyl.

In an embodiment, the 5-membered heteroaryl group of R^{e2} is substituted with 0 or 1 substituents independently selected from C₁-C₄ alkyl, C₁-C₄ hydroxyalkyl, C₁-C₄ haloalkyl, C₃-C₆ heterocyclyl and C₃-C₆ cycloalkyl optionally substituted with one or two instances of halo or methyl.

In an embodiment, the 5-membered heteroaryl group of R^{e2} is substituted with 0 or 1 substituents independently selected from methyl, ethyl, isopropyl, tert-butyl, difluoromethyl, 1,1-difluoroethyl, —C(OH)(CH₃)₂, oxetan-3-yl, cyclopropyl, 1-methylcyclopropyl and 2-fluorocyclopropyl.

In an embodiment, the 5 membered heteroaryl group of R^{e2} is substituted with 0 or 1 substituents independently selected from -Me, -Et, -iPr, -tBu, —C(OH)(CH₃)₂, —CHF₂, —CF₂CH₃, —CH(F)CH₃, —CF₃, oxetanyl, cyclopropyl, 1-Me-cyclopropyl and 2-F-cyclopropyl.

In an embodiment, R^{e2} is selected from the group consisting of:

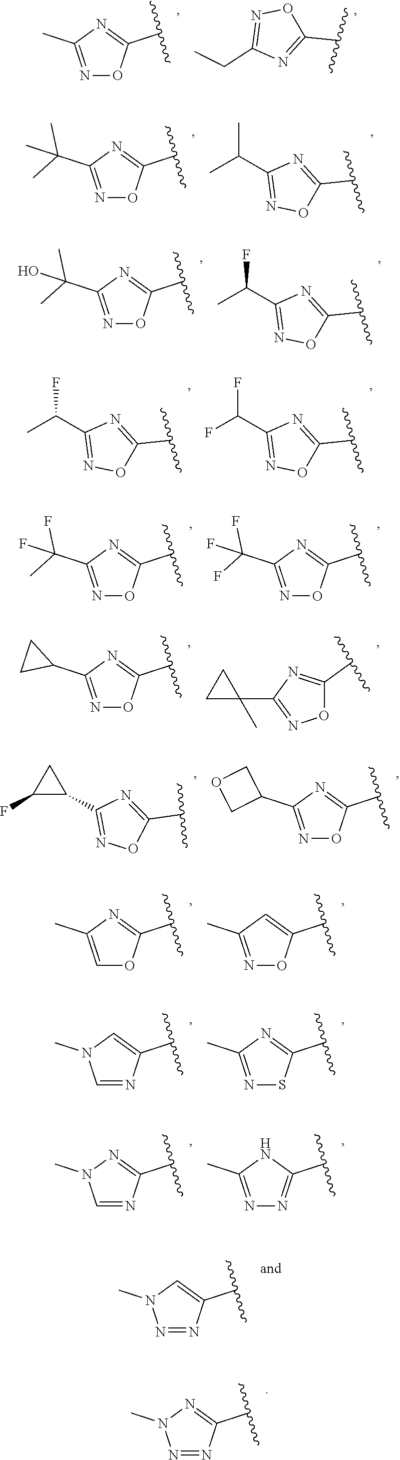

In an embodiment, R^{e2} is selected from the group consisting of:

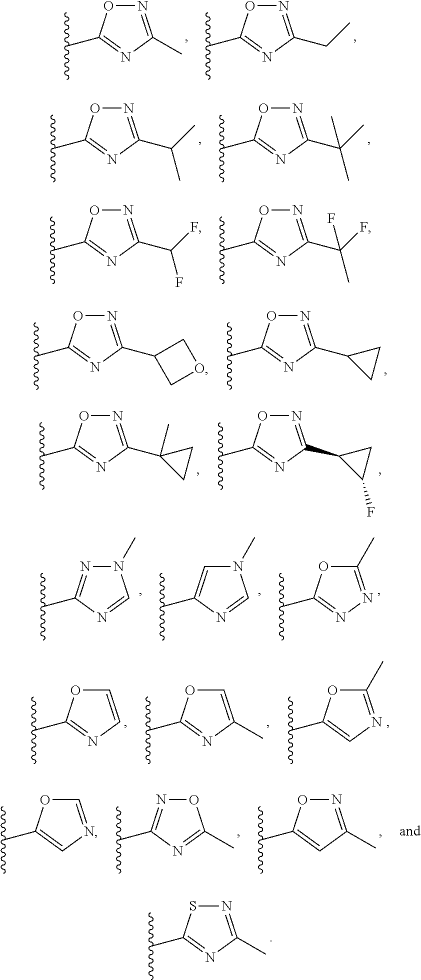

In an embodiment, R^{e2} is selected from the group consisting of:

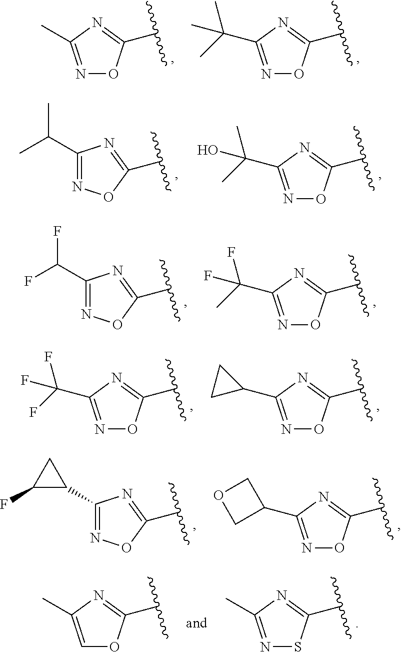

In an embodiment, R^{e2} is 1,2,4-oxadiazolyl substituted with 1 substituent selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

In an embodiment, R^{e2} is

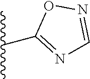 [SMILES: *c1ncno1]

substituted with 1 substituent selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃—C₆ heterocyclyl and C₃-C₆ cycloalkyl optionally substituted with one or two instances of halo or methyl.

In an embodiment, the oxadiazolyl is substituted with one substituent selected from methyl, ethyl, isopropyl, tert-butyl, difluoromethyl, 1,1-difluoroethyl, —C(OH)(CH₃)₂, oxetan-3-yl, cyclopropyl, 1-methylcyclopropyl and 2-fluorocyclopropyl.

In an embodiment, R^{e2} is selected from the group consisting of:

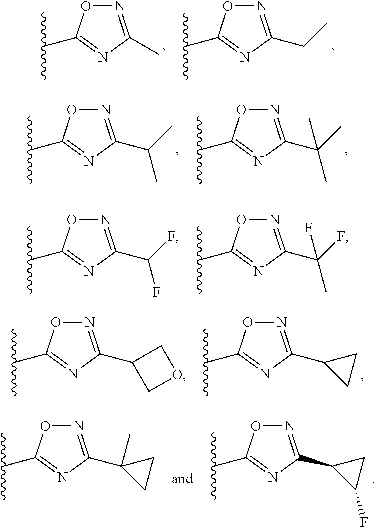

In an embodiment, R^{e2} is selected from the group consisting of:

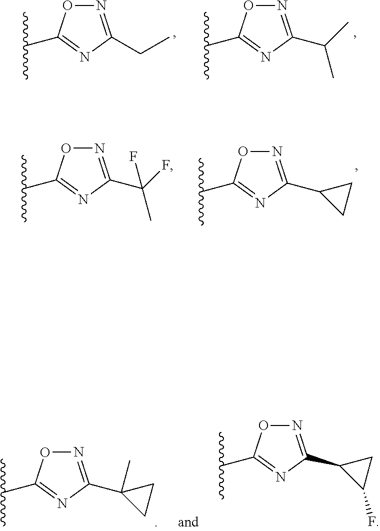

In an embodiment, R^{e2} is

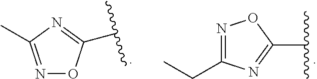

In an embodiment, R^{e2} is

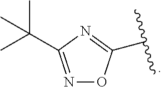 [SMILES: *c1nc(C(C)(C)C)no1]

In an embodiment, R^{e2} is

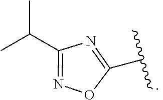 [SMILES: *c1nc(C(C)C)no1]

In an embodiment, R^{e2} is

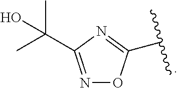 [SMILES: *c1nc(C(C)(C)O)no1]

In an embodiment, R^{e2} is

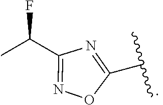 [SMILES: *c1nc([C@@H](C)F)no1]

In an embodiment, R^{e2} is

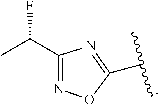 [SMILES: *c1nc([C@H](C)F)no1]

In an embodiment R^{e2}

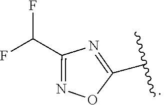 [SMILES: *c1nc(C(F)F)no1]

In an embodiment, R^{e2} is

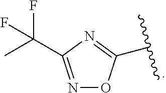 [SMILES: *c1nc(C(C)(F)F)no1]

In an embodiment, R^{e2} is

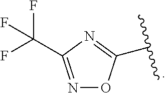 [SMILES: *c1nc(C(F)(F)F)no1]

In an embodiment, R^{e2} is

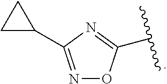 [SMILES: *c1nc(C2CC2)no1]

In an embodiment, R^{e2} is

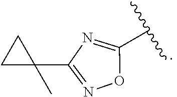 [SMILES: *c1nc(C2(C)CC2)no1]

In an embodiment, R^{e2} is

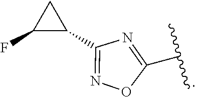 [SMILES: *c1nc([C@H]2C[C@@H]2F)no1]

In an embodiment, R^{e2} is

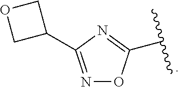 [SMILES: *c1nc(C2COC2)no1]

In an embodiment, R^{e2} is

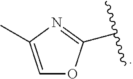 [SMILES: *c1nc(C)co1]

In an embodiment, R^{e2} is

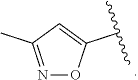 [SMILES: *c1cc(C)no1]

In an embodiment, R^{e2} is

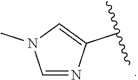 [SMILES: *c1cn(C)cn1]

In an embodiment, R^{e2} is

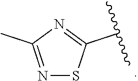 [SMILES: *c1nc(C)ns1]

In an embodiment, R^{e2} is

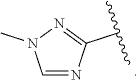 [SMILES: *c1ncn(C)n1]

In an embodiment, R^{e2} is

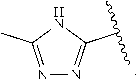 [SMILES: *c1nnc(C)[nH]1]

In an embodiment, R^{e2} is

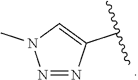 [SMILES: *c1cn(C)nn1]

In an embodiment, R^{e2} is

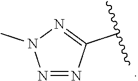 [SMILES: *c1nnn(C)n1]

As generally defined herein, R^{e3} is —NR⁵R⁶, wherein R⁵ and R⁶ are as defined in any of the embodiments described herein. In an embodiment, R^{e3} is selected from

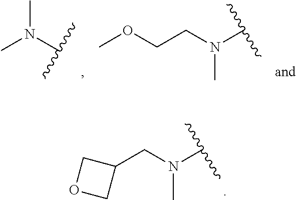

In an embodiment, R^{e3} is

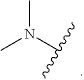 [SMILES: *N(C)C]

As generally defined herein, R is selected from the group consisting of H, halo, C₁-C₄ alkyl, C₁-C₄ alkoxy, C₁-C₄ haloalkyl and C₁-C₄ haloalkoxy. In an embodiment, R is C₁-C₄ alkyl. In an embodiment, R is methyl.

As generally defined herein, each R⁹ is independently selected from the group consisting of hydrogen, halo, —NH₂, C₁-C₄ alkyl, C₁-C₄ alkoxy, C₁-C₄ haloalkyl, C₁-C₄ haloalkoxy and C₂-C₃ alkynyl. In an embodiment, R⁹ is —NH₂.

As generally defined herein, each R^{h} is independently selected from the group consisting of hydrogen, halo, C₁-C₄ alkyl, C₁-C₄ alkoxy, C₁-C₄ haloalkyl, C₁-C₄ haloalkoxy, C₁-C₄ alkenyl and C₂-C₃ alkynyl. In an embodiment, each R^{h} is independently selected from the group consisting of hydrogen, halo, C₁-C₄ alkyl, C₁-C₄ alkoxy, C₁-C₄ haloalkyl, C₁-C₄ haloalkoxy and C₂-C₃ alkynyl. In an embodiment, R^{h} is selected from the group consisting of hydrogen, —F, and —C₁. In an embodiment, R^{h} is selected from the group consisting of hydrogen and —F. In an embodiment, R^{h} is hydrogen.

As generally defined herein, each R, is independently selected from the group consisting of hydrogen, halo, C₁-C₄ alkyl, C₁-C₄ alkoxy, C₁-C₄ haloalkyl, C₁-C₄ haloalkoxy, C₁-C₄ alkenyl and C₂-C₃ alkynyl. In an embodiment, Rⁱ is independently selected from the group consisting of hydrogen, halo, C₁-C₄ alkyl, C₁-C₄ alkoxy, C₁-C₄ haloalkyl, C₁-C₄ haloalkoxy and C₂-C₃ alkynyl. In an embodiment, Rⁱ is selected from the group consisting of hydrogen, —F, and —Cl. In an embodiment, Rⁱ is selected from the group consisting of —F and —Cl. In an embodiment, Rⁱ is —Cl. In an embodiment, Rⁱ is —F.

As generally defined herein, each R^{j} is independently selected from the group consisting of hydrogen, halo, C₁-C₄ alkyl, C₁-C₄ alkoxy, C₁-C₄ haloalkyl, C₁-C₄ haloalkoxy, C₁-C₄ alkenyl and C₂-C₃ alkynyl. In an embodiment, each R^{j} is independently selected from the group consisting of hydrogen, halo, C₁-C₄ alkyl, C₁-C₄ alkoxy, C₁-C₄ haloalkyl, C₁-C₄ haloalkoxy and C₂-C₃ alkynyl. In an embodiment, R^{j} is selected from the group consisting of C₁-C₄ acyclic alkyl, C₃-C₄ cycloalkyl, C₁-C₄ alkenyl and C₁-C₄ haloalkyl. In an embodiment, R^{j} is selected from the group consisting of hydrogen, halo, C₁-C₄ acyclic alkyl, C₃-C₄ cycloalkyl, C₁-C₄ alkenyl and C₁-C₄ haloalkyl. In an embodiment, R^{j} is selected from the group consisting of hydrogen, halo, C₃-C₄ cycloalkyl and C₁-C₄ haloalkyl. In an embodiment, R^{j} is selected from the group consisting of hydrogen, —F, —Cl, -Me, -Et, cyclopropyl, cyclobutyl, prop-1-en-2-yl, —CHF₂ and —CF₃. In an embodiment, R^{j} is selected from the group consisting of —F, —Cl, cyclopropyl and —CF₃. In an embodiment, R^{j} is selected from the group consisting of hydrogen, —F, —Cl and —CHF₂. In an embodiment, R^{j} is selected from the group consisting of C₃-C₄ cycloalkyl and C₁-C₄ haloalkyl. In an embodiment, R^{j} is selected from the group consisting of -Me, -Et, cyclopropyl, cyclobutyl, prop-1-en-2-yl, —CHF₂ and —CF₃. In an embodiment, R^{j} is selected from the group consisting of cyclopropyl and —CF₃. In an embodiment, R^{j} is cyclopropyl. In an embodiment, R^{j} is —CF₃. In an embodiment, R^{j} is difluoromethyl.

As generally defined herein, each R^{k} is independently selected from the group consisting of hydrogen, halo, C₁-C₄ alkyl, C₁-C₄ alkoxy, C₁-C₄ haloalkyl, C₁-C₄ haloalkoxy, C₁-C₄ alkenyl and C₂-C₃ alkynyl. In an embodiment, each R^{k} is independently selected from the group consisting of hydrogen, halo, C₁-C₄ alkyl, C₁-C₄ alkoxy, C₁-C₄ haloalkyl, C₁-C₄ haloalkoxy and C₂-C₃ alkynyl. In an embodiment, R^{k} is selected from the group consisting of hydrogen, halo, C₁-C₄ acyclic alkyl, C₃-C₄ cycloalkyl, C₁-C₄ alkenyl and C₁-C₄ haloalkyl. In an embodiment, R^{k} is selected from the group consisting of hydrogen, halo, C₃-C₄ cycloalkyl and C₁-C₄ haloalkyl. In an embodiment, R^{k} is selected from the group consisting of hydrogen, —F, —Cl, -Me, -Et, cyclopropyl, cyclobutyl, prop-1-en-2-yl, —CHF₂ and —CF₃. In an embodiment, R^{k} is selected from the group consisting of —F, —Cl, cyclopropyl and —CF₃. In an embodiment, R^{k} is selected from the group consisting of hydrogen, —F, —Cl and —CHF₂. In an embodiment, R^{k} is selected from the group consisting of hydrogen and halo. In an embodiment, R^{k} is selected from the group consisting of hydrogen, —F and —Cl. In an embodiment, R^{k} is —F. In an embodiment, R^{k} is —Cl. In an embodiment, R^{k} is hydrogen.

As generally defined herein, each R^{m} is independently selected from the group consisting of hydrogen, halo, —NH₂, C₁-C₄ alkyl, C₁-C₄ alkoxy, C₁-C₄ haloalkyl, C₁-C₄ haloalkoxy and C₂—C₃ alkynyl. In an embodiment, R^{m} is selected from the group consisting of hydrogen, halo, —NH₂, C₁-C₄ alkyl, C₁-C₄ alkoxy, C₁-C₄ haloalkyl, C₁-C₄ haloalkoxy and C₂-C₃ alkynyl. In an embodiment, R^{m} is hydrogen.

As generally defined herein, each Rⁿ is independently selected from the group consisting of hydrogen, halo, C₁-C₄ alkyl, C₁-C₄ alkoxy, C₁-C₄ haloalkyl, C₁-C₄ haloalkoxy, C₁-C₄ alkenyl and C₂-C₃ alkynyl. In an embodiment, each Rⁿ is independently selected from the group consisting of hydrogen, halo, C₁-C₄ alkyl, C₁-C₄ alkoxy, C₁-C₄ haloalkyl, C₁-C₄ haloalkoxy and C₂-C₃ alkynyl. In an embodiment, Rⁿ is selected from the group consisting of hydrogen, methyl and trifluoromethyl. In an embodiment, Rⁿ is trifluoromethyl.

As generally defined herein, each R^{o} is independently selected from the group consisting of hydrogen, halo, C₁-C₄ alkyl, C₁-C₄ alkoxy, C₁-C₄ haloalkyl, C₁-C₄ haloalkoxy, C₁-C₄ alkenyl and C₂-C₃ alkynyl. In an embodiment, each R^{o} is independently selected from the group consisting of hydrogen, halo, C₁-C₄ alkyl, C₁-C₄ alkoxy, C₁-C₄ haloalkyl, C₁-C₄ haloalkoxy and C₂-C₃ alkynyl. In an embodiment, R^{o} is selected from the group consisting of hydrogen, methyl and trifluoromethyl. In an embodiment, R^{o} is methyl. In an embodiment, R^{o} is fluoro.

As generally defined herein, each R^{p} is independently selected from the group consisting of hydrogen, halo, —NH₂, C₁-C₄ alkyl, C₁-C₄ alkoxy, C₁-C₄ haloalkyl, C₁-C₄ haloalkoxy and C₂-C₃ alkynyl. In an embodiment, R^{p} is —NH₂.

In an embodiment, Ring A is selected from the group consisting of:

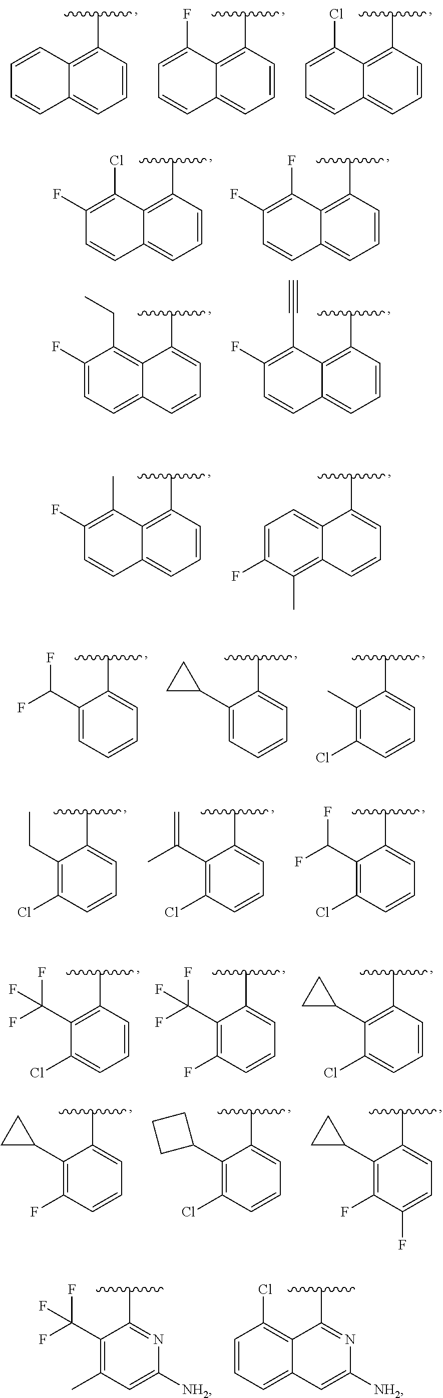

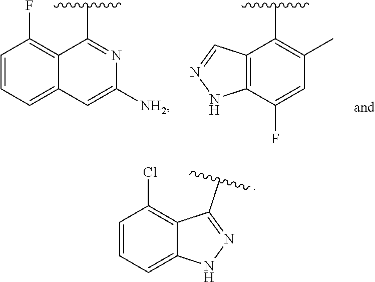 [SMILES: *c1nc(N)cc2cccc(F)c12 ; *c1c(C)cc(F)c2[nH]ncc12 ; *c1n[nH]c2cccc(Cl)c12]

In an embodiment, Ring A is selected from the group consisting of:

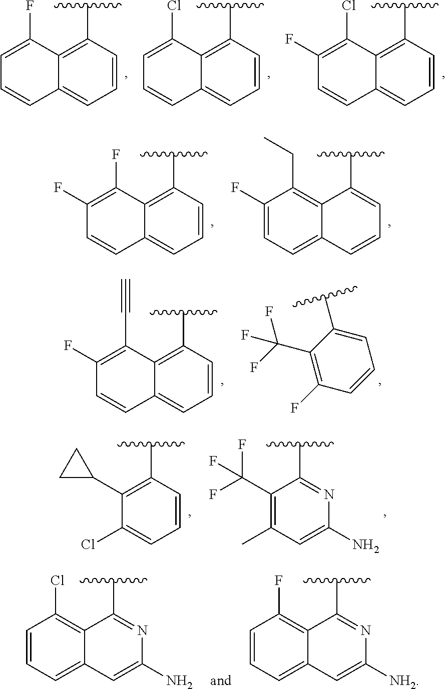

In an embodiment, Ring A is selected from the group consisting of:

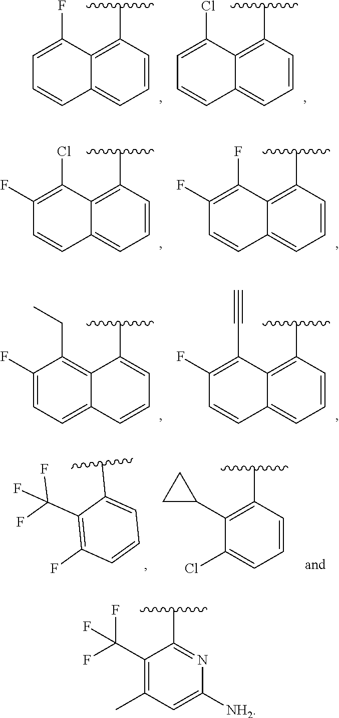

In an embodiment, Ring A is selected from the group consisting of:

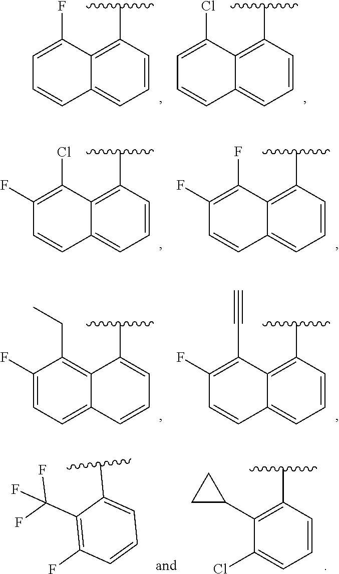

In an embodiment, Ring A is selected from the group consisting of:

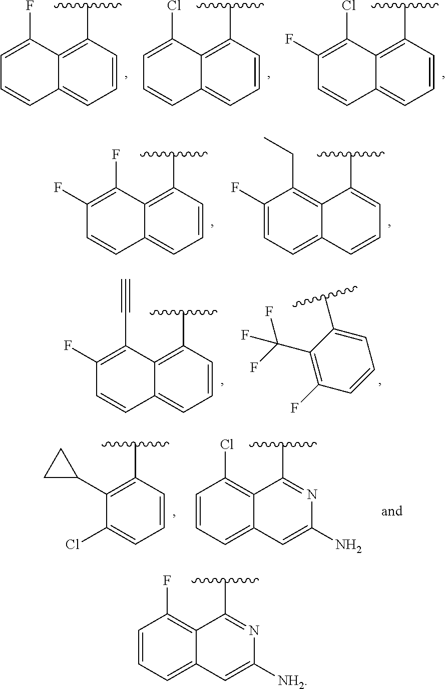

In an embodiment, the compound is of Formula I or Formula II:

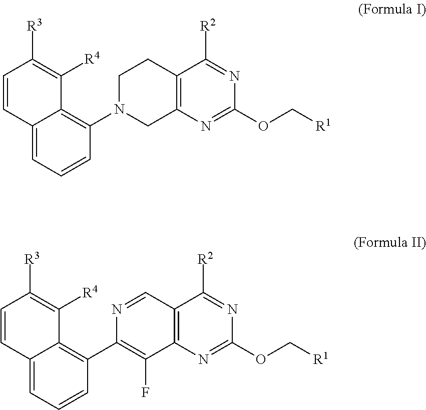

or a salt thereof; and/or an isotopologue thereof, wherein R¹, R², R³ and R⁴ are as defined in any of the embodiments described herein.

In an embodiment, the compound is of Formula I. In an embodiment, the compound is of Formula II.

In an embodiment, the compound is of Formula III or Formula IV:

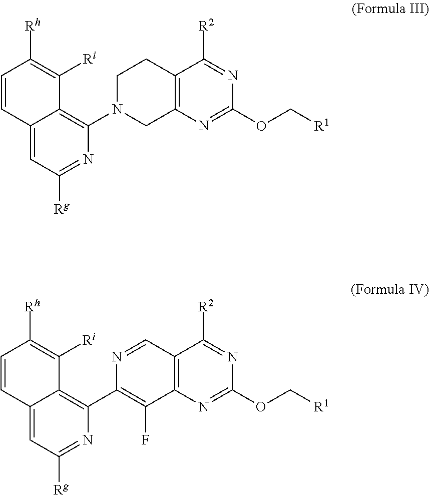

or a salt thereof; and/or an isotopologue thereof, wherein each R¹, R², R^{h}, Rⁱ and R^{g} are as defined in any of the embodiments described herein.

In an embodiment, the compound is of Formula III. In an embodiment, the compound is of Formula IV.

In an embodiment, the compound is of Formula V or Formula VI:

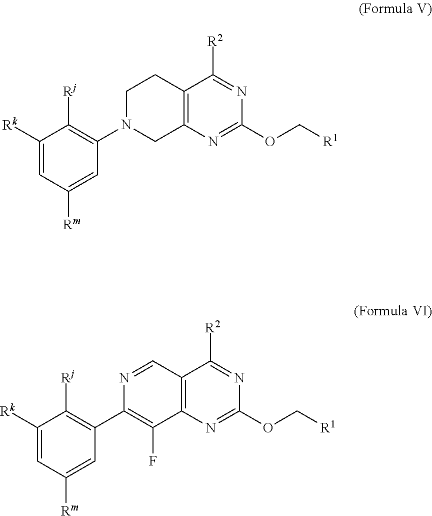

or a salt thereof; and/or an isotopologue thereof, wherein R¹, R², R^{j}, R^{k} and R^{m} are as defined in any of the embodiments described herein.

In an embodiment, the compound is of Formula V. In an embodiment, the compound is of Formula VI.

In an embodiment, the compound is of Formula VII, Formula VIII or Formula IX:

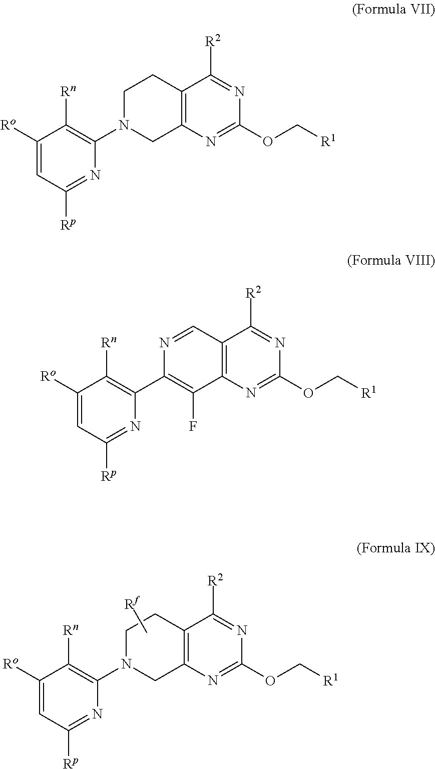

or a salt thereof; and/or an isotopologue thereof, wherein R¹, R², R^{f}, Rⁿ, R^{o} and R^{p} are as defined in any of the embodiments described herein.

In an embodiment, the compound is of Formula VII. In an embodiment, the compound is of Formula VIII. In an embodiment, the compound is of Formula IX. In an embodiment, the compound is of Formula IXa:

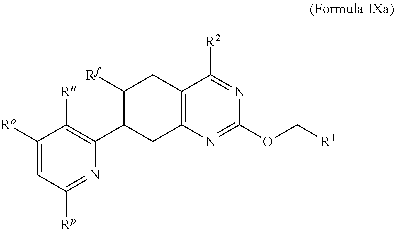

or a salt thereof; and/or an isotopologue thereof, wherein R¹, R², R^{f}, Rⁿ, R^{o} and R^{p} are as defined in any of the embodiments described herein.

### Methods For Treatment of Cancer

The compounds of Formula (A), Formula (B) and Formula (C), and pharmaceutically acceptable salts and/or isotopologues thereof, including embodiments thereof disclosed herein, are useful for the treatment of cancer, which include but are not limited to, various types of cancer including e.g., lung, colorectal, pancreatic, bile duct, thyroid, gall bladder, uterine, mesothelioma, cervical, and bladder cancers. More particularly, cancers that may be treated by the compounds of Formula (A), Formula (B) and Formula (C), and pharmaceutically acceptable salts and/or isotopologues thereof, including embodiments thereof disclosed herein, include, but are not limited to cancers such as glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma. In some embodiments, including any of the foregoing embodiments, the cancer is a KRAS G12C mediated cancer. In some embodiments, including any of the foregoing embodiments, the subject has been diagnosed as having a KRAS G12C mediated cancer. In some embodiments, including any of the foregoing embodiments, the subject has been determined to be at risk of developing a KRAS G12C mediated cancer.

In an aspect, provided is a compound of Formula (A), Formula (B) or Formula (C) as described in any of the embodiments described herein or a pharmaceutical formulation as described in any of the embodiments described herein for use as a medicament.

In an aspect, provided is a compound of Formula (A), Formula (B) or Formula (C) as described in any of the embodiments described herein or a pharmaceutical formulation as described in any of the embodiments described herein for use in treating or suppressing cancer. In an embodiment, when the compound is a salt, the salt is a pharmaceutically acceptable salt. In an embodiment, the cancer is selected from the group consisting of: lung, colorectal, pancreatic, bile duct, thyroid, gall bladder, uterine, mesothelioma, cervical, and bladder cancers. In an embodiment, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma. In an embodiment, the cancer is a KRAS G12C mediated cancer. In an embodiment, the subject has been diagnosed as having a KRAS G12C mediated cancer. In an embodiment, the compound or pharmaceutical composition is configured for administration with a therapeutically effective amount of an additional chemotherapeutic agent. In an embodiment, the compound or pharmaceutical composition is configured for administration in a therapeutically effective amount.

In an aspect, provided is a compound of Formula (A), Formula (B) or Formula (C) as described in any of the embodiments described herein or a pharmaceutical formulation as described in any of the embodiments described herein for use in the manufacturing of a medicament for treating or suppressing cancer, wherein when the compound is a salt, the salt is a pharmaceutically acceptable salt. In an embodiment, the cancer is selected from the group consisting of: lung, colorectal, pancreatic, bile duct, thyroid, gall bladder, uterine, mesothelioma, cervical, and bladder cancers. In an embodiment, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma. In an embodiment, the cancer is a KRAS G12C mediated cancer. In an embodiment, the subject has been diagnosed as having a KRAS G12C mediated cancer. In an embodiment, the compound or pharmaceutical composition is configured for administration with a therapeutically effective amount of an additional chemotherapeutic agent. In an embodiment, the medicament comprises a therapeutically effective amount of the compound or pharmaceutical composition.

In an aspect, provided is a use of a compound of Formula (A), Formula (B) or Formula (C) as described in any of the embodiments described herein or a pharmaceutical formulation as described in any of the embodiments described herein in the manufacturing of a medicament for treating or suppressing cancer, wherein when the compound is a salt, the salt is a pharmaceutically acceptable salt. In an embodiment, the cancer is selected from the group consisting of: lung, colorectal, pancreatic, bile duct, thyroid, gall bladder, uterine, mesothelioma, cervical, and bladder cancers. In an embodiment, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma. In an embodiment, the cancer is a KRAS G12C mediated cancer. In an embodiment, the subject has been diagnosed as having a KRAS G12C mediated cancer. In an embodiment, the compound or pharmaceutical composition is configured for administration with a therapeutically effective amount of an additional chemotherapeutic agent. In an embodiment, the medicament comprises a therapeutically effective amount of the compound or pharmaceutical composition.

In an aspect, provided is a use of a compound of Formula (A), Formula (B) or Formula (C) as described in any of the embodiments described herein or a pharmaceutical formulation as described in any of the embodiments described herein for treating or suppressing cancer, wherein when the compound is a salt, the salt is a pharmaceutically acceptable salt.

In an embodiment, the cancer is selected from the group consisting of: lung, colorectal, pancreatic, bile duct, thyroid, gall bladder, uterine, mesothelioma, cervical, and bladder cancers. In an embodiment, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma. In an embodiment, the cancer is a KRAS G12C mediated cancer. In an embodiment, the subject has been diagnosed as having a KRAS G12C mediated cancer. In an embodiment, the compound or pharmaceutical composition is configured for administration with a therapeutically effective amount of an additional chemotherapeutic agent. In an embodiment, use involves a therapeutically effective amount of the compound or composition.

The compounds of Formula (A), Formula (B) and Formula (C), and pharmaceutically acceptable salts and/or isotopologues thereof, including embodiments thereof disclosed herein, may be used for methods for inhibiting KRAS G12C in a cell, by contacting the cell in which inhibition of KRAS G12C activity is desired with an amount of the compound effective to inhibit KRAS G12C activity. Inhibition may be partial or total. In some embodiments, the contacting is in vitro. In some embodiments, the contacting is in vivo.

### Testing

The compounds of Formula (A), Formula (B) and Formula (C), and pharmaceutically acceptable salts and/or isotopologues thereof, including embodiments thereof disclosed herein, may be tested by, for example, methods described in the Examples below, or by known and generally accepted cell and/or animal models.

The ability of compounds of Formula (A), Formula (B) and Formula (C), and pharmaceutically acceptable salts and/or isotopologues thereof, to inhibit activity of the GTP-bound form of KRAS G12C can be tested using methods such as the in vitro assay described in Examples 116 and 117 below. Example 116 describes determining, for various compounds, the half-maximal inhibition (IC₅₀) of KRAS G12C loaded with GTP analogue GMPPNP from binding to cRaf, as the Ras-binding domain (RBD). Example 117 describes determining, for various compounds, the half-maximal inhibition (IC₅₀) of KRAS G12C loaded with GTP analogue GMPPNP from binding to PI3Kα, as the Ras-binding domain (RBD). Example 120 describes testing compounds for the ability to inhibit cell viability in MCF10A G12C/A59G mutant, which abrogates GTPase activity, thus preventing hydrolysis of GTP to GDP.

### Pharmaceutical Compositions

In general, the compounds of Formula (A), Formula (B) and Formula (C), and pharmaceutically acceptable salts and/or isotopologues thereof, of this disclosure (also may be referred to herein as “compounds” or “compounds of this disclosure”) will be administered in a therapeutically effective amount by any of the accepted modes of administration for agents that serve similar utilities. Therapeutically effective amounts of compounds of this disclosure may range from about 0.01 to about 500 mg per kg patient body weight per day, which can be administered in single or multiple doses. In some embodiments, a suitable dosage level may be from about 0.1 to about 250 mg/kg per day; or about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of a compound of this disclosure, i.e., the active ingredient, will depend upon numerous factors such as the severity of the disease to be treated, the age and relative health of the patient, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance.

The compositions are comprised of in general, a compound of this disclosure in combination with at least one pharmaceutically acceptable excipient. Acceptable excipients are non-toxic, aid administration, and do not adversely affect the therapeutic benefit of the compound of this disclosure. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds of the disclosure may be administered topically, that is by non-systemic administration. This includes the application of the compounds externally to the epidermis or the buccal cavity and the instillation of such compounds into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.10% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator. Other suitable pharmaceutical excipients and their formulations are described in Remington's Pharmaceutical Sciences, edited by E. W. Martin (Mack Publishing Company, 20th ed., 2000).

The level of the compound in a formulation can vary within the full range employed by those skilled in the art. Typically, the formulation will contain, on a weight percent (wt. %) basis, from about 0.01-99.99 wt. % of a compound of this disclosure based on the total formulation, with the balance being one or more suitable pharmaceutical excipients. For example, the compound is present at a level of about 1-80 wt. %.

### Combinations and Combination Therapies

The compounds of this disclosure may be used in combination with one or more other drugs in the treatment of diseases or conditions for which compounds of this disclosure or the other drugs may have utility. Such other drug(s) may be administered contemporaneously or sequentially with a compound of the present disclosure. When a compound of this disclosure is used contemporaneously with one or more other drugs, a pharmaceutical composition in unit dosage form containing such other drugs and the compound of the present disclosure is contemplated. However, the combination therapy may also include therapies in which the compound of this disclosure and one or more other drugs are administered on different overlapping schedules. It is also contemplated that when used in combination with one or more other active ingredients, the compounds of the present disclosure and the other active ingredients may be used in lower doses than when each is used singly.

Accordingly, the pharmaceutical compositions of the present disclosure also include those that contain one or more other drugs, in addition to a compound of the present disclosure.

The above combinations include combinations of a compound of this disclosure not only with one other drug, but also with two or more other active drugs. Likewise, a compound of this disclosure may be used in combination with other drugs that are used in the prevention, treatment, control, amelioration, or reduction of risk of the diseases or conditions for which a compound of this disclosure is useful. Such other drugs may be administered contemporaneously or sequentially with a compound of the present disclosure. When a compound of this disclosure is used contemporaneously with one or more other drugs, a pharmaceutical composition containing such other drugs in addition to the compound of this disclosure can be used. Accordingly, the pharmaceutical compositions of the present disclosure also include those that also contain one or more other active ingredients, in addition to a compound of this disclosure. The weight ratio of the compound of this disclosure to the second active ingredient may be varied and will depend upon the effective dose of each ingredient. Generally, a therapeutically effective dose of each will be used.

Where the subject in need is suffering from or at risk of suffering from cancer, the subject can be treated with a compound of this disclosure in any combination with one or more other anti-cancer agents.

In some embodiments, the compounds of the present disclosure are used in combination with a CDK 4/6 inhibitor. Examples of CDK 4/6 inhibitors suitable for the provided compositions and methods include, but are not limited to, abemaciclib (N-(5-((4-ethylpiperazin-1-yl)methyl)pyridin-2-yl)-5-fluoro-4-(4-fluoro-1-isopropyl-2-methyl-1H-benzo[d]imidazol-6-yl)pyrimidin-2-amine); palbociclib (6-acetyl-8-cyclopentyl-5-methyl-2-((5-(piperazin-1-yl)pyridin-2-yl)amino)-pyrido[2,3-d]pyrimidin-7(8H)-one) and ribociclib (7-cyclopentyl-N,N-dimethyl-2-((5-(piperazin-1-yl)pyridin-2-yl)amino)-7H-pyrrolo[2,3-d]pyrimidine-6-carboxamide) whereas the CDK 4/6 inhibitor trilaciclib (2′-((5-(piperazin-1-yl)pyridin-2-yl)amino)-7′,8′-dihydro-6′H-spiro-[cyclohexane-1,9′-pyrazino[1′,2′:1,5]pyrrolo[2,3-d]pyrimidin]-6′-one) is in late stage clinical trials. Another CDK 4/6 inhibitor useful in the methods herein is the CDK 2/4/6 inhibitor PF-06873600 (pyrido[2,3-d]pyrimidin-7(8H)-one, 6-(difluoromethyl)-8-[(1R,2R)-2-hydroxy-2-methylcyclopentyl]-2-[[1-(methylsulfonyl)-4-piperidinyl]amino]).

In another embodiment the compounds of the present disclosure are used in combination with Raf family kinase inhibitors. Examples of Raf family kinase inhibitors suitable for the provided compositions and methods include, but are not limited to, encorafenib (LGX818): methyl (S)-(1-((4-(3-(5-chloro-2-fluoro-3-(methylsulfonamido)phenyl)-1-isopropyl-1H-pyrazol-4-yl)pyrimidin-2-yl)amino)propan-2-yl)carbamate; PLX-8394: N-(3-(5-(2-cyclopropylpyrimidin-5-yl)-3a,7a-dihydro-1H-pyrrolo[2,3-b]pyridine-3-carbonyl)-2,4-difluorophenyl)-3-fluoropyrrolidine-1-sulfonamide; Raf-709: N-(2-methyl-5′-morpholino-6′-((tetrahydro-2H-pyran-4-yl)oxy)-[3,3′-bipyridin]-5-yl)-3-(trifluoromethyl)benzamide; LXH254: N-(3-(2-(2-hydroxyethoxy)-6-morpholinopyridin-4-yl)-4-methylphenyl)-2-(trifluoromethyl)isonicotinamide; Sorafenib: 4-(4-(3-(4-chloro-3-(trifluoromethyl)phenyl)ureido)phenoxy)-N-methylpicolinamide; L Y 3009120: 1-(3,3-dimethylbutyl)-3-(2-fluoro-4-methyl-5-(7-methyl-2-(methylamino)pyrido-[2,3-d]pyrimidin-6-yl)phenyl)urea; Lifirafenib (BGB-283); 5-(((1R,1aS,6bS)-1-(6-(trifhioro-methyl)-1H-benzo[d]imidazol-2-yl)-1a,6b-dihydro-1H-cyclopropa[b]benzofuran-5-yl)methyl)-3,4-dihydro-1,8-naphthyridin-2(1H)-one; Tak-632: N-(7-cyano-6-(4-fluoro-3-(2-(3-(trifluoromethyl)-phenyl)acetamido)phenoxy)benzo[d]thiazol-2-yl)cyclopropanecarboxamide; CEP-32496: 1-(3-((6,7-dimethoxyquinazolin-4-yl)oxy)phenyl)-3-(5-(1,1,1-trifluoro-2-methylpropan-2-yl)isoxazol-3-yl)urea; CCT196969: 1-(3-(tert-butyl)-1-phenyl-1H-pyrazol-5-yl)-3-(2-fluoro-4-((3-oxo-3,4-dihydropyrido[2,3-b]pyrazin-8-yl)oxy)phenyl)urea; and R05126766: N-[3-fluoro-4-[[4-methyl-2-oxo-7-(2-pyrimidinyloxy)-2H-1-benzopyran-3-yl]methyl]-2-pyridinyl]-N′-methylsulfamide.

In another embodiment the compounds of the present disclosure are used in combination with Src family kinases. Examples of Src family kinase inhibitors suitable for the provided compositions and methods include, but are not limited to, Dasatinib (N-(2-chloro-6-methylphenyl)-2-((6-(4-(2-hydroxyethyl)piperazin-1-yl)-2-methylpyrimidin-4-yl)amino)thiazole-5-carboxamide); Ponatinib (3-(imidazo[1,2-b]pyridazin-3-ylethynyl)-4-methyl-N-(4-((4-methylpiperazin-1-yl)methyl)-3-(trifluoromethyl)phenyl)benzamide); Vandetanib (N-(4-bromo-2-fluorophenyl)-6-methoxy-7-((1-methylpiperidin-4-yl)methoxy)quinazolin-4-amine); Bosutinib (4-((2,4-dichloro-5-methoxyphenyl)amino)-6-methoxy-7-(3-(4-methylpiperazin-1-yl)-propoxy)quinoline-3-carbonitrile); Saracatinib (N-(5-chlorobenzo[d][1,3]dioxol-4-yl)-7-(2-(4-methylpiperazin-1-yl)ethoxy)-5-((tetrahydro-2H-pyran-4-yl)oxy)quinazolin-4-amine); KX2-391 (N-benzyl-2-(5-(4-(2-morpholinoethoxy)phenyl)pyridin-2-yl)acetamide); SU6656 ((Z)—N,N-dimethyl-2-oxo-3-((4,5,6,7-tetrahydro-1H-indol-2-yl)methylene)indoline-5-sulfonamide); PP1 (1-(tert-butyl)-3-(p-tolyl)-1H-pyrazolo[3,4-d]pyrimidin-4-amine); WH-4-023 (2,6-dimethylphenyl (2,4-dimethoxyphenyl)(2-((4-(4-methylpiperazin-1-yl)phenyl)amino)pyrimidin-4-yl)carbamate) and KX-01 (N-benzyl-2-(5-(4-(2-morpholinoethoxy)phenyl)pyridin-2-yl)acetamide). In one embodiment, the Src inhibitor is Dasatinib. In one embodiment, the Src inhibitor is Saracatinib. In one embodiment, the Src inhibitor is Ponatinib. In one embodiment, the Src inhibitor is Vandetanib. In one embodiment, the Src inhibitor is KX-01.

In another embodiment the compounds of the present disclosure are used in combination with a SHP-2 inhibitor which include, but are not limited to SHP-099 (6-(4-amino-4-methylpiperidin-1-yl)-3-(2,3-dichlorophenyl)pyrazine-2-amine dihydrochloride), RMC-4550 (3(3S,4S)-(4-amino-3-methyl-2-oxa-8-azaspiro[4.5]decan-8-yl)-6-(2,3-dichlorophenyl)pyrazin-2-yl)methanol), RMC-4360 (Revolution Medicines), TN0155 (Novartis), BBP-398 (BridgeBio), and ERAS-601 (Erasca).

In another embodiment the compounds of the present disclosure are used in combination with an mTOR inhibitor. Examples of mTOR inhibitors suitable for the provided compositions and methods include, but are not limited to, Everolimus, Rapamycin, Zotarolimus (ABT-578), ridaforolimus (Deforolimus; MK-8669), Sapanisertib (INK128; 5-(4-amino-1-isopropyl-1H-pyrazolo[3,4-d]pyrimidin-3-yl)benzo[d]oxazol-2-amine), Torin-1; 1-(4-(4-propionylpiperazin-1-yl)-3-(trifluoromethyl)cyclohexyl)-9-(quinolin-3-yl)benzo[h][1,6]naphthyridin-2(1H)-one, dactolisib (BEZ235); 2-methyl-2-(4-(3-methyl-2-oxo-8-(quinolin-3-yl)-2,3-dihydro-1H-imidazo[4,5-c]quinolin-1-yl)phenyl)propanenitrile, buparlisib (5-(2,6-dimorpholin-4-ylpyrimidin-4-yl)-4-(trifluoromethyl)pyridin-2-amine); GDC-0941 (pictilisib); 4-[2-(1H-indazol-4-yl)-6-[(4-methylsulfonylpiperazin-1-yl)methyl]thieno[3,2-d]pyrimidin-4-yl]morpholine); GDC-0349 ((S)-1-ethyl-3-(4-(4-(3-methylmorpholino)-7-(oxetan-3-yl)-5,6,7,8-tetrahydropyrido[3,4-d]pyrimidin-2-yl)phenyl)urea), VS-5584 (SB2343) (5-(8-methyl-2-morpholin-4-yl-9-propan-2-ylpurin-6-yl)pyrimidin-2-amine) and vistusertib (AZD-2014; 3-(2,4-bis((S)-3-methylmorpholino)pyrido-[2,3-d]pyrimidin-7-yl)-N-methylbenzamide).

In another embodiment the compounds of the present disclosure are used in combination with a pan ErbB family inhibitor. In one embodiment the KRAS and pan ErbB family inhibitors are the only active agents in the provided compositions and methods. In one embodiment, the pan ErbB family inhibitor is an irreversible inhibitor. Examples of irreversible pan ErbB family inhibitors suitable for the provided compositions and methods include, but are not limited to, Afatinib; Dacomitinib; Canertinib; Poziotinib, AV 412 (N-4-([3-(chloro-4-fluorophenyl)amino]-7-[3-methyl-3-(4-methyl-1-piperazin-1-butyn-1-yl]-6-quinazolinyl]-2-prepenamide); PF 6274484 N-4-([3-(chloro-4-fluorophenyl)amino]-7-methoxy-6-quinazolinyl]-2-propenamide) and HKI 357 N-(2(E)-N-[[4-[[3-chloro-4-[(fluorophenyl)methoxy]phenyl]amino]-3-cyano-7-ethoxy-6-quinolinyl]-4-(dimethylamino)-2-butenamide). In another embodiment, the pan ErbB family inhibitor is a reversible inhibitor. Examples of reversible pan ErbB family inhibitors suitable for the provided compositions and methods include, but are not limited to erlotinib, gefitinib, sapitinib; varlitinib; TAK-285 (N-[2-[4-[3-chloro-4-[3-(trifluoromethyl)phenoxy]phenylamino]-5H-pyrrolo[3,2-d]pyrimidin-5-yl]ethyl]-3-hydroxy-3-methylbutanamide); AEE788 (S)-(6-(4-((4-ethylpiperazin-1-ylmethyl)phenyl]-N-(1-phenylethyl)-7H-pyrrolo[2,3-d]pyrimidin-4-amine); tarloxotinib 3-[N-[4-(3-bromo-4-chlorophenylamino)-pyrido[3,4-d]pyrimidin-6-yl]carbamoyl]-N,N-dimethyl-N-(1-methyl-4-nitro-1H-imidazol-5-ylmethyl)-2(E)-propen-1-aminium bromide); BMS 599626 ((3S)—3-morpholinylmethyl-[4-[[1-[(3-fluorophenyl)methyl]-1H-indazol-5-yl]amino]-5-methylpurrolo[2,1-f][1,2,4]triazine-6-yl]carbamate dihydrochloride); and GW 583340 (N-[3-chloro-4-(3-fluorobenzyloxy)phenyl]-6-[2-[2-(methylsulfonyl)ethylaminomethyl]thiazol-4-yl]quinazolin-4-amine dihydrochloride).

In one embodiment, the pan ErbB family inhibitor is a combination of an EGFR inhibitor and a HER2 inhibitor, wherein the EGFR inhibitor and the HER2 inhibitor are a combination of two of AG 1478 (N-(3-chlorophenyl)-6,7-dimethoxyquinazolin-4-amine hydrochloride); AG 555 ((E)-2-cyano-3-(3,4-dihydoxyphenyl)-N-(3-phenylpropyl)-2-propenamide); AG 556 ((E)-2-cyano-3-(3,4-dihydroxyphenyl)-N-(4-phenylbutyl)-2-propenamide; AG 825 (E-3-[3-benzothiazol-2-ylsulfanylmethyl)-4-hydroxy-5-methoxyphenyl]-2-cyano-2-propenamide); CP 724714 (2-methoxy-N-[(2E)-3-[4-[3-methyl-4-(6-methylpyridin-3-yloxy)phenylamino]quinazolin-6-yl]-2-propen-1-yl]acetamide; BIBU 1361 (N-(3-chloro-4-fluorophenyl)-6-[4-(diethylaminomethyl)-piperidin-1-yl]pyrimido[5,4-d]pyrimidin-4-amine dihydrochloride); BIBU 1382; (N⁸-(3-chloro-4-fluorophenyl)-N²-(1-methyl-4-piperidinyl)pyrimidino[5,4-d]pyrimidin-4-amine dihydrochloride), JNJ 28871063 (5E-4-amino-6-[4-(benzyloxy)-3-chlorophenylamino]-pyrimidine-5-carbaldehyde N-[2-(4-morpholinyl)ethyl]oxime hydrochloride); PD 153035 (4-(3-bromophenylamino)-6,7-dimethoxyquinazoline hydrochloride); and PD 158780 (N⁴-(3-bromophenyl)-N⁶-methyl-pyrido[3,4-d]pyrimidine-4,6-diamine).

In one embodiment, the pan ErbB family inhibitor is an anti-EGFR antibody, an anti-HER2 antibody or combination of an anti-EGFR antibody and anti-HER2 antibody. Antibodies, including monoclonal antibodies, antibody conjugates and bispecific antibodies, targeting EGFR and/or HER2 are well known and several antibodies are commercially available for research and human clinical use. Examples of anti-EGFR antibodies suitable for the provided compositions and methods include necitumumab, panitumumab and cetuximab. Examples of anti-HER2 antibodies suitable for the provided compositions and methods include, pertuzumab, trastuzumab, and trastuzumab emtansine.

In some embodiments, the compounds of the present disclosure are used in combination with an immune checkpoint inhibitor. Examples of immune checkpoint inhibitors suitable for the provided compositions and methods include, but are not limited to, PD-1, PD-L1, CTLA-4, and LAG-3 inhibitors, such as Pembrolizumab (Keytruda®), Nivolumab (Opdivo®), Cemiplimab (Libtayo®), Atezolizumab (Tecentriq®), Avelumab (Bavencio®), Durvalumab (Imfinzi™), Ipilimumab (Yervoy®), Relatlimab, Opdualag, and Dostarlimab (Jemperli).

The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

### SELECTED EMBODIMENTS

Embodiment 1. A compound of Formula (A), Formula (B) or Formula (C)

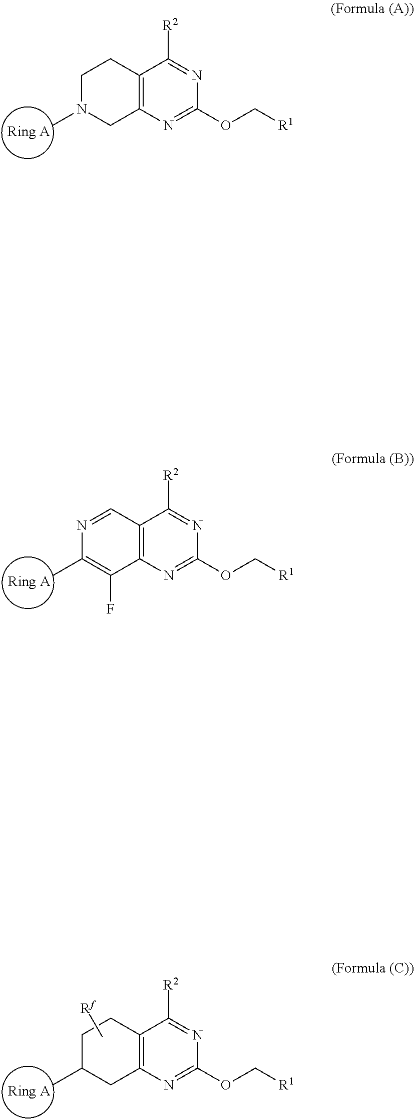

or a salt thereof; and/or an isotopologue thereof, wherein:
Ring A is a 6-10 membered aryl or a 5-10 membered heteroaryl, each substituted with 0, 1, 2 or 3 substituents independently selected from halo, —OH, —NH₂, C₁-C₄ alkyl, C₁-C₄ alkenyl, C₁-C₄ alkoxy, C₁-C₄ haloalkyl, C₁-C₄ haloalkoxy and C₂-C₃ alkynyl;
R¹ is

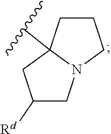

R^{d} is H, —F or —OH;
R² is R^{2c};
R^{2c} is

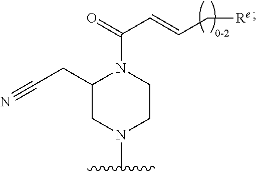

R^{e} is R^{e1}, R^{e2} or R^{e3};
R^{e1} is a 4-10 membered heterocycle which is substituted with 0, 1, 2, 3 or 4 substituents independently selected from halo, hydroxy, C₁-C₄ alkyl, C₁-C₆ aminoalkyl, 4-6 membered heterocycle, —C(O)C₁-C₆ alkyl, C₁-C₆ alkoxy, C₁-C₆ alkoxyalkoxy, C₁-C₄ haloalkyl, C₁-C₄ haloalkoxy and C₂-C₃ alkynyl;
R^{e2} is a 5-6 membered heteroaryl wherein the attachment point is a carbon atom group and wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl;
R^{e3} is —NR⁵R⁶, wherein each R⁵ and R⁶ is independently selected from C₁-C₄ alkyl, C₁-C₆ alkoxy and —CH₂-(4-6 membered heterocycle); and
R^{f} is selected from the group consisting of H, halo, C₁-C₄ alkyl, C₁-C₄ alkoxy, C₁-C₄ haloalkyl and C₁-C₄ haloalkoxy.
Embodiment 2. A compound of Formula (A), Formula (B) or Formula (C)

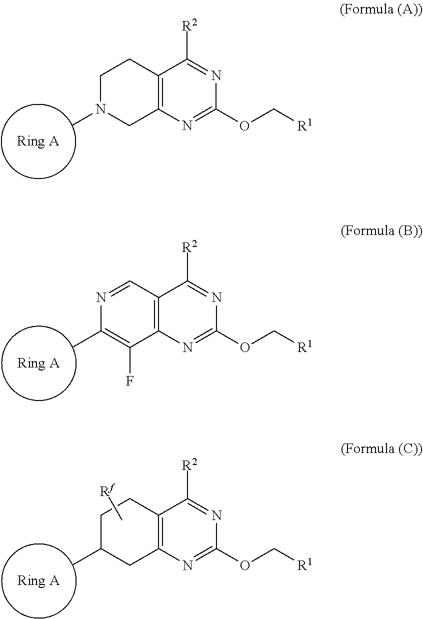

or a salt thereof; and/or an isotopologue thereof; wherein:
Ring A is a 6-10 membered aryl or a 5-10 membered heteroaryl, each substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl;
R¹ is

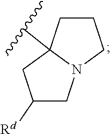

R^{d} is H or F;
R² is R^{2c};
R^{2c} is

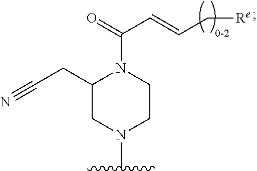

R^{e} is R^{e1} or R^{e2};
R^{e1} is a 4-10 membered heterocycle which is substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl;
R^{e2} is a 5-6 membered heteroaryl wherein the attachment point is a carbon atom group and wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl; and
R^{f} is selected from the group consisting of H, halo, C₁-C₄ alkyl, C₁-C₄ alkoxy, C₁-C₄ haloalkyl and C₁-C₄ haloalkoxy.
Embodiment 3. The compound of embodiment 1 or 2 wherein R^{2c} is selected from the group consisting of:

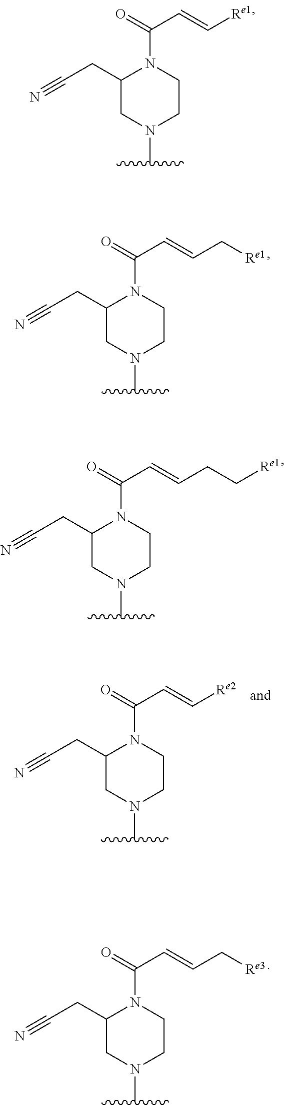

Embodiment 4. The compound of embodiment 1 or 2, wherein R^{2c} is selected from the group consisting of:

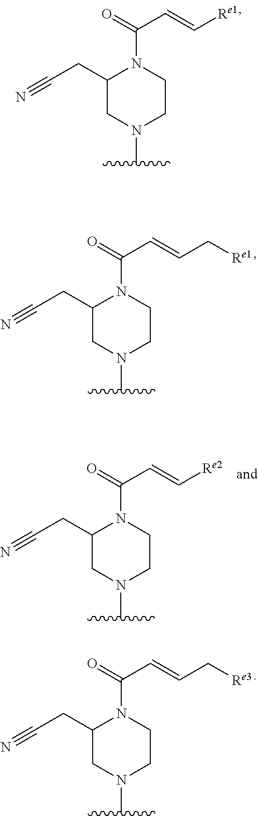

Embodiment 5. The compound of any one of embodiments 1 to 4, wherein the compound is of Formula (A) or Formula (B).
Embodiment 6. The compound of any one of embodiments 1 to 4, wherein the compound is of Formula (A) or Formula (C).
Embodiment 7. The compound of any one of embodiments 1 to 4, wherein the compound is of Formula (B) or Formula (C).
Embodiment 8. The compound of any one of embodiments 1 to 4, wherein the compound is of Formula (A).
Embodiment 9. The compound of any one of embodiments 1 to 4, wherein the compound is of Formula (B).
Embodiment 10. The compound of any one of embodiments 1 to 4, wherein the compound is of Formula (C).
Embodiment 11. The compound of any one of embodiments 1 to 10, wherein Ring A is selected from the group consisting of naphthalenyl, phenyl, isoquinolinyl, pyridinyl and 1-H-indazolyl, each substituted with 0, 1, 2 or 3 substituents independently selected from halo, —OH, —NH₂, C₁-C₄ alkyl, C₁-C₄ alkoxy, C₁-C₄ alkenyl, C₁-C₄ haloalkyl, C₁-C₄ haloalkoxy and C₂-C₃ alkynyl.
Embodiment 12. The compound of embodiment 11, wherein the naphthalenyl, phenyl, isoquinolinyl, pyridinyl and 1-H-indazolyl are independently substituted with 0, 1, 2 or 3 substituents independently selected from halo, —NH₂, C₁-C₄ acyclic alkyl, C₃-C₄ cycloalkyl, C₁-C₄ alkenyl, C₁-C₄ haloalkyl and C₂-C₃ alkynyl.
Embodiment 13. The compound of embodiment 11, wherein the naphthalenyl, phenyl, isoquinolinyl, pyridinyl and 1-H-indazolyl are independently substituted with 0, 1, 2 or 3 substituents independently selected from —F, —Cl, —OH, —NH₂, -Me, -Et, —Pr, -ⁱPr, cyclopropyl, cyclobutyl, vinyl, prop-1-en-2-yl, —CHF₂, —CH₂F, —CF₃, —OMe, -OEt, —OCHF₂, —OCF₃ and ethynyl.
Embodiment 14. The compound of embodiment 11, wherein the naphthalenyl, phenyl, isoquinolinyl, pyridinyl and 1-H-indazolyl are independently substituted with 0, 1, 2 or 3 substituents independently selected from —F, —Cl, —NH₂, -Me, -Et, cyclopropyl, cyclobutyl, prop-1-en-2-yl, —CHF₂, —CF₃, and ethynyl.
Embodiment 15. The compound of any one of embodiments 1 to 10, wherein Ring A is selected from the group consisting of naphthalen-1-yl, phenyl, isoquinolin-1-yl, pyridin-2-yl, 1-H-indazol-3-yl and 1-H-indazol-4-yl, each substituted with 0, 1, 2 or 3 substituents independently selected from halo, —OH, —NH₂, C₁-C₄ alkyl, C₁-C₄ alkoxy, C₁-C₄ alkenyl, C₁-C₄ haloalkyl, C₁-C₄ haloalkoxy and C₂-C₃ alkynyl.
Embodiment 16. The compound of embodiment 15, wherein the naphthalen-1-yl, phenyl, isoquinolin-1-yl, pyridin-2-yl, 1-H-indazol-3-yl and 1-H-indazol-4-yl are independently substituted with 0, 1, 2 or 3 substituents independently selected from halo, —NH₂, C₁-C₄ acyclic alkyl, C₃-C₄ cycloalkyl, C₁-C₄ alkenyl, C₁-C₄ haloalkyl and C₂-C₃ alkynyl.
Embodiment 17. The compound of embodiment 15, wherein the naphthalen-1-yl, phenyl, isoquinolin-1-yl, pyridin-2-yl, 1-H-indazol-3-yl and 1-H-indazol-4-yl are independently substituted with 0, 1, 2 or 3 substituents independently selected from —F, —Cl, —OH, —NH₂, -Me, -Et, —Pr, -ⁱPr, cyclopropyl, cyclobutyl, vinyl, prop-1-en-2-yl, —CHF₂, —CH₂F, —CF₃, —OMe, -OEt, —OCHF₂, —OCF₃ and ethynyl.
Embodiment 18. The compound of embodiment 15, wherein the naphthalen-1-yl, phenyl, isoquinolin-1-yl, pyridin-2-yl, 1-H-indazol-3-yl and 1-H-indazol-4-yl are independently substituted with 0, 1, 2 or 3 substituents independently selected from —F, —Cl, —NH₂, -Me, -Et, cyclopropyl, cyclobutyl, prop-1-en-2-yl, —CHF₂, —CF₃, and ethynyl.
Embodiment 19. The compound of embodiment 15, wherein the naphthalen-1-yl, phenyl, isoquinolin-1-yl, pyridin-2-yl, 1-H-indazol-3-yl and 1-H-indazol-4-yl are independently substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl.
Embodiment 20. The compound of any one of embodiments 1 to 10, wherein Ring A is selected from the group consisting of naphthalenyl, phenyl and pyridinyl, each substituted with 0, 1, 2 or 3 substituents independently selected from halo, —OH, —NH₂, C₁-C₄ alkyl, C₁-C₄ alkoxy, C₁-C₄ haloalkyl, C₁-C₄ alkenyl, C₁-C₄ haloalkoxy and C₂-C₃ alkynyl.
Embodiment 21. The compound of embodiment 20, wherein the naphthalenyl, phenyl and pyridinyl are independently substituted with 0, 1, 2 or 3 substituents independently selected from halo, —NH₂, C₁-C₄ acyclic alkyl, C₃-C₄ cycloalkyl, C₁-C₄ alkenyl, C₁-C₄ haloalkyl and C₂-C₃ alkynyl.
Embodiment 22. The compound of embodiment 20, wherein the naphthalenyl, phenyl and pyridinyl are independently substituted with 0, 1, 2 or 3 substituents independently selected from —F, —Cl, —NH₂, -Me, -Et, cyclopropyl, cyclobutyl, prop-1-en-2-yl, —CHF₂, —CF₃, and ethynyl.
Embodiment 23. The compound of any one of embodiments 20 to 22, wherein the naphthalenyl is naphthalen-1-yl and the pyridinyl is pyridin-2-yl.
Embodiment 24. The compound of any one of embodiments 1 to 10, wherein Ring A is selected from the group consisting of naphthalenyl, phenyl, isoquinolinyl and pyridinyl, each substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl.
Embodiment 25. The compound of any one of embodiments 1 to 10, wherein Ring A is selected from the group consisting of naphthalen-1-yl, phenyl, isoquinolin-1-yl and pyridin-2-yl, each substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl.
Embodiment 26. The compound of any one of embodiments 1 to 10, wherein Ring A is selected from the group consisting of:

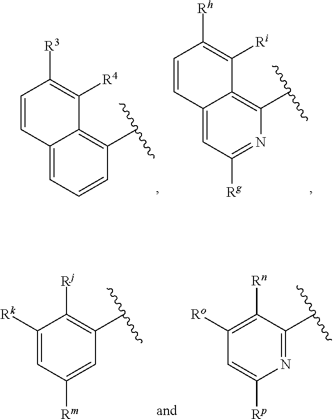

wherein:
each R³, R⁴, R^{h}, Rⁱ, R^{j}, R^{k}, Rⁿ and R^{o} is independently selected from the group consisting of hydrogen, halo, C₁-C₄ alkyl, C₁-C₄ alkoxy, C₁-C₄ haloalkyl, C₁-C₄ haloalkoxy, C₁-C₄ alkenyl and C₂-C₃ alkynyl; and
each R^{g}, R^{m} and R^{p} is independently selected from the group consisting of hydrogen, halo, —NH₂, C₁-C₄ alkyl, C₁-C₄ alkoxy, C₁-C₄ haloalkyl, C₁-C₄ haloalkoxy and C₂-C₃ alkynyl.
Embodiment 27. The compound of any one of embodiments 1 to 10, wherein Ring A is selected from the group consisting of:

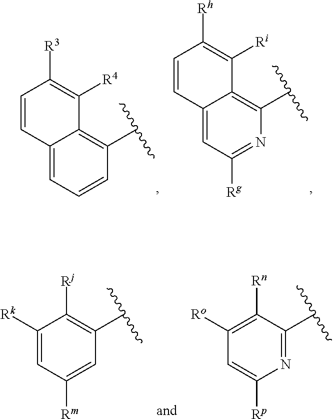

wherein:
each R³, R⁴, R^{h}, Rⁱ, R^{j}, R^{k}, Rⁿ and R^{o} is independently selected from the group consisting of hydrogen, halo, C₁-C₄ alkyl, C₁-C₄ alkoxy, C₁-C₄ haloalkyl, C₁-C₄ haloalkoxy and C₂-C₃ alkynyl; and
each R^{g}, R^{m} and R^{p} is independently selected from the group consisting of hydrogen, halo, —NH₂, C₁-C₄ alkyl, C₁-C₄ alkoxy, C₁-C₄ haloalkyl, C₁-C₄ haloalkoxy and C₂-C₃ alkynyl.
Embodiment 28. The compound of embodiment 26 or 27, wherein Ring A is selected from the group consisting of:

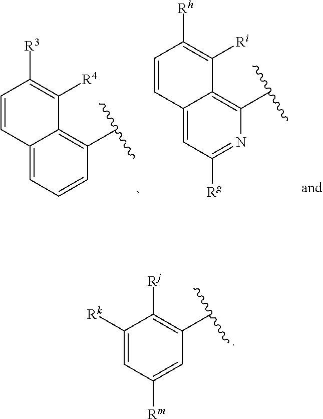

Embodiment 29. The compound of embodiment 26 or 27, wherein Ring A is selected from the group consisting of:

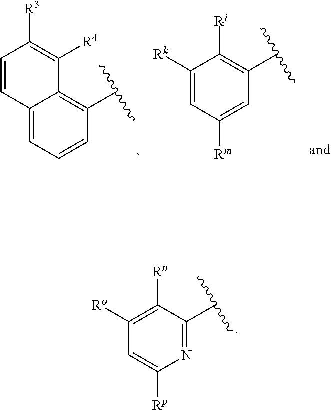

Embodiment 30. The compound of embodiment 26 or 27, wherein Ring A is selected from the group consisting of:

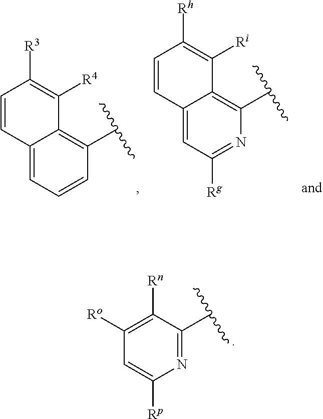

Embodiment 31. The compound of embodiment 26 or 27, wherein Ring A is selected from the group consisting of:

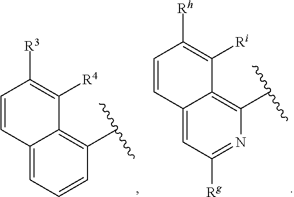

Embodiment 32. The compound of embodiment 26 or 27, wherein Ring A is selected from the group consisting of:

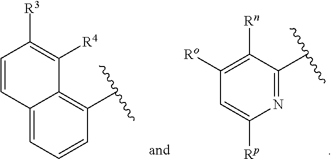

Embodiment 33. The compound of embodiment 26 or 27, wherein Ring A is selected from the group consisting of:

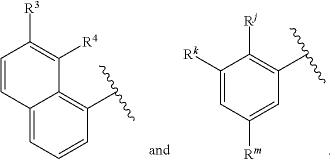

Embodiment 34. The compound of embodiment 26 or 27, wherein Ring A is:

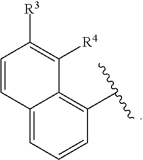

Embodiment 35. The compound of embodiment 26 or 27, wherein Ring A is:

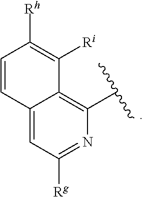

Embodiment 36. The compound of embodiment 26 or 27, wherein Ring A is:

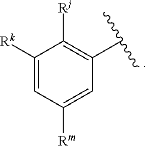

Embodiment 37. The compound of embodiment 26 or 27, wherein Ring A is:

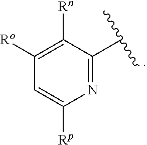

Embodiment 38. The compound of any one of embodiments 1 to 10, wherein Ring A is selected from the group consisting of:

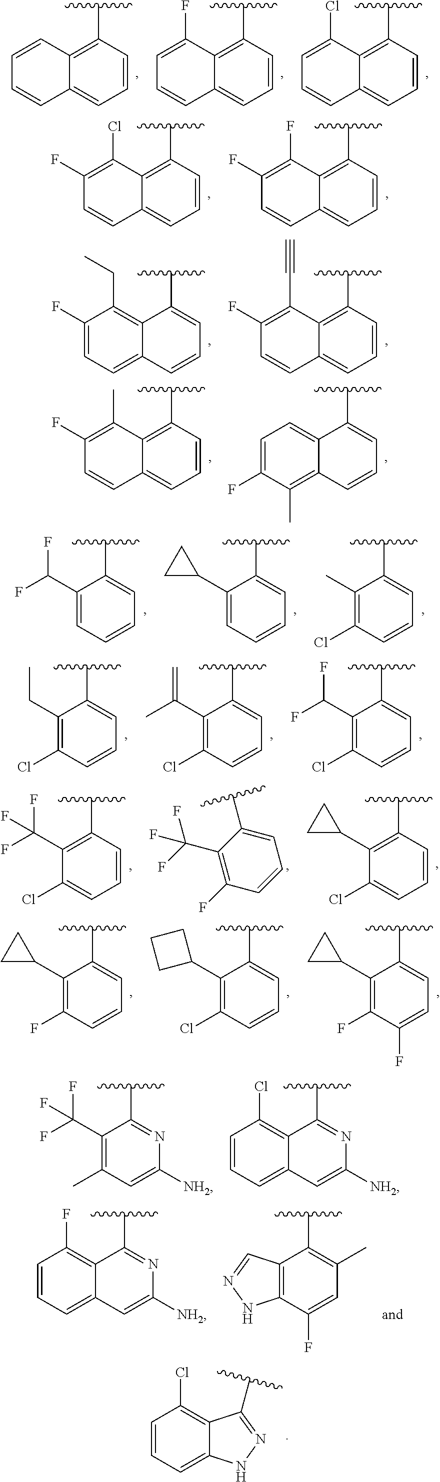

Embodiment 39. The compound of any one of embodiments 1 to 10, wherein Ring A is selected from the group consisting of:

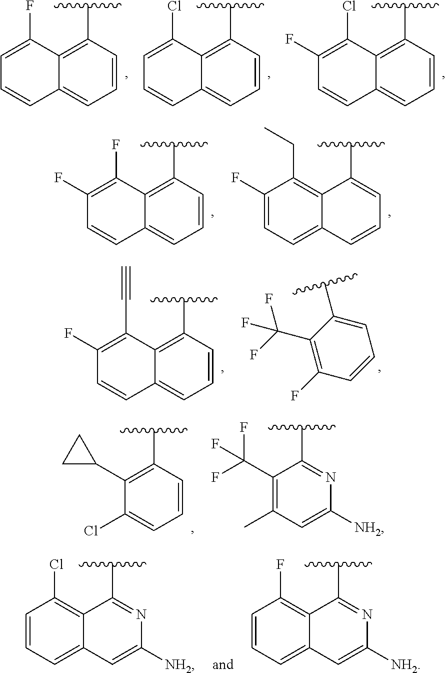

Embodiment 40. The compound of any one of embodiments 1 to 10, wherein Ring A is selected from the group consisting of:

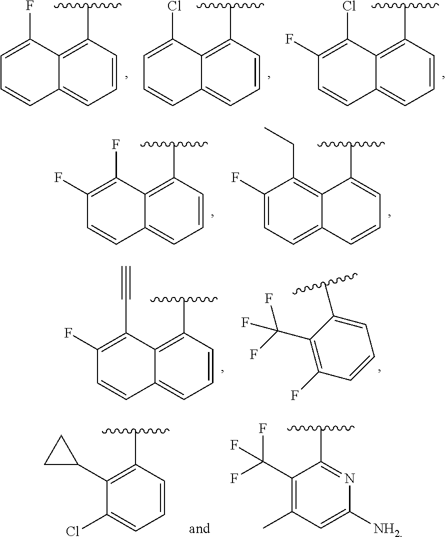

Embodiment 41. The compound of any one of embodiments 1 to 10, wherein Ring A is selected from the group consisting of:

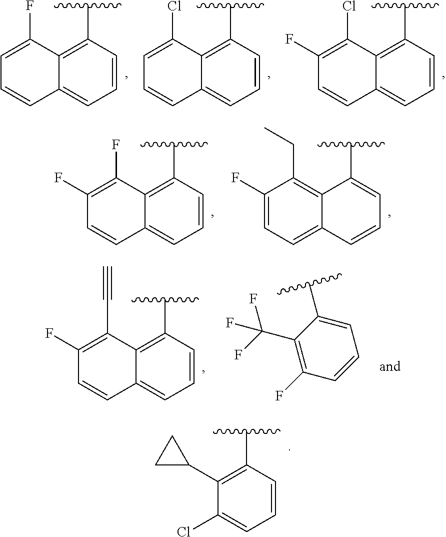

Embodiment 42. The compound ofany one of embodiments 1 to 10, wherein Ring A is selected from the group consisting of:

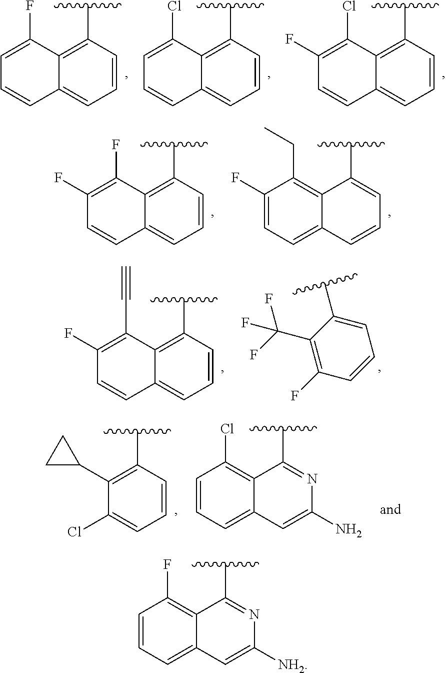

Embodiment 43. The compound of any one of embodiments 1 to 10, wherein Ring A is naphthalenyl substituted with 0, 1, 2 or 3 substituents independently selected from halo, hydroxy, C₁-C₄ alkyl, C₁-C₄ alkoxy, C₁-C₄ haloalkyl, C₁-C₄ haloalkoxy and C₂-C₃ alkynyl.
Embodiment 44. The compound of embodiment 43, wherein the naphthalenyl is substituted with 0, 1 or 2 substituents independently selected from halo, C₁-C₄ acyclic alkyl and C₂-C₃ alkynyl.
Embodiment 45. The compound of embodiment 43, wherein the naphthalenyl is substituted with 0, 1 or 2 substituents independently selected from —F, —Cl, -Me, -Et and ethynyl.
Embodiment 46. The compound of embodiment 43, wherein the naphthalenyl is unsubstituted.
Embodiment 47. The compound of embodiment 43, wherein the naphthalenyl is substituted with 1 or 2 substituents independently selected from —F, —Cl, -Et and ethynyl.
Embodiment 48. The compound of embodiment 43, wherein the naphthalenyl is substituted with 1 or 2 substituents independently selected from —F and —Cl.
Embodiment 49. The compound of embodiment 43, wherein the naphthalenyl is substituted with 1 or 2 instances of —F.
Embodiment 50. The compound of embodiment 43, wherein the naphthalenyl is substituted with 1 or 2 instances of —Cl.
Embodiment 51. The compound of any one of embodiments 43 to 50, wherein the naphthalenyl is naphthalen-1-yl.
Embodiment 52. The compound of any one of embodiments 1 to 10, wherein Ring A is selected from the group consisting of:

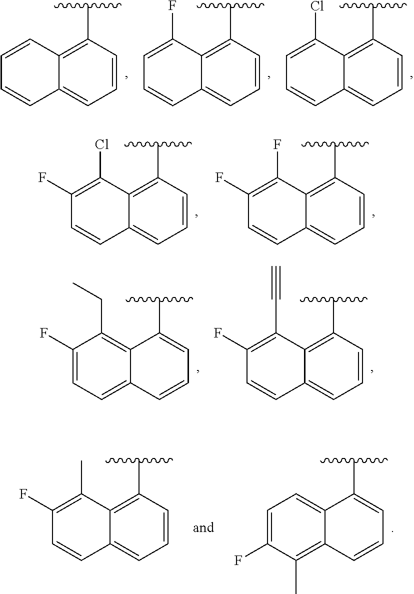

Embodiment 53. The compound of any one of embodiments 1 to 10, wherein Ring A is selected from the group consisting of:

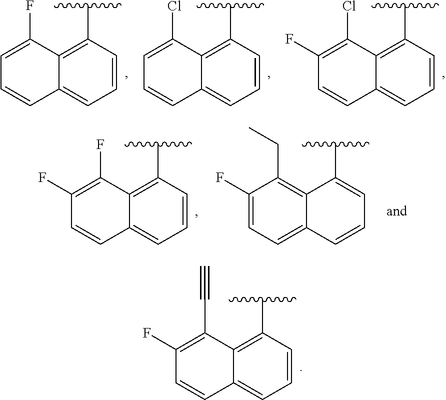

Embodiment 54. The compound of any one of embodiments 1 to 10, wherein Ring A is selected from the group consisting of:

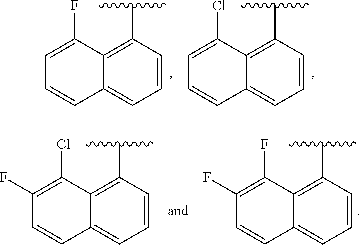

Embodiment 55. The compound of any one of embodiments 1 to 10, wherein Ring A is

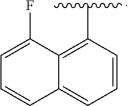 [SMILES: *c1cccc2cccc(F)c12 ; "."]

Embodiment 56. The compound of any one of embodiments 1 to 10, wherein Ring A is

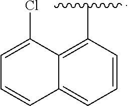 [SMILES: *c1cccc2cccc(Cl)c12 ; "."]

Embodiment 57. The compound of any one of embodiments 1 to 10, wherein Ring A is

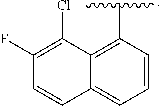 [SMILES: *c1cccc2ccc(F)c(Cl)c12 ; "."]

Embodiment 58. The compound of any one of embodiments 1 to 10, wherein Ring A is

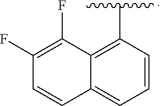 [SMILES: *c1cccc2ccc(F)c(F)c12 ; "."]

Embodiment 59. The compound of any one of embodiments 1 to 10, wherein Ring A is

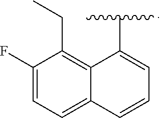 [SMILES: *c1cccc2ccc(F)c(CC)c12 ; "."]

Embodiment 60. The compound of any one of embodiments 1 to 10, wherein Ring A is

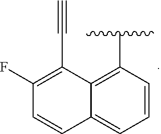 [SMILES: *c1cccc2ccc(F)c(C#C)c12]

Embodiment 61. The compound of embodiment 1 or 2, wherein the compound is of Formula I or Formula II:

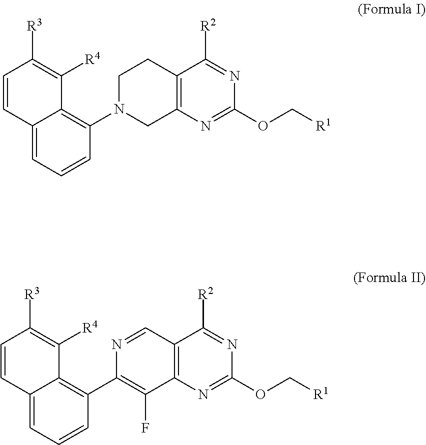

or a salt thereof; and/or an isotopologue thereof, wherein:
R³ is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl; and
R⁴ is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl.
Embodiment 62. The compound of embodiment 61, wherein the compound is of Formula I.
Embodiment 63. The compound of embodiment 61, wherein the compound is of Formula II.
Embodiment 64. The compound of any one of embodiments 26 to 34 and 61 to 63, wherein R³ and R⁴ are independently selected from the group consisting of hydrogen, fluoro, and chloro.
Embodiment 65. The compound of any one of embodiments 26 to 34 and 61 to 63, wherein R³ is selected from the group consisting of hydrogen —F and —Cl.
Embodiment 66. The compound of any one of embodiments 26 to 34 and 61 to 63, wherein R³ is selected from the group consisting of hydrogen and —F.
Embodiment 67. The compound of any one of embodiments 26 to 34 and 61 to 63, wherein R³ is —F.
Embodiment 68. The compound of any one of embodiments 26 to 34 and 61 to 63, wherein R³ is hydrogen.
Embodiment 69. The compound of any one of embodiments 26 to 34 and 61 to 63, wherein R³ and R⁴ are independently selected from the group consisting of hydrogen, halo, C₁-C₄ alkyl and C₂-C₃ alkynyl.
Embodiment 70. The compound of any one of embodiments 26 to 34 and 61 to 63, wherein R³ and R⁴ are independently selected from the group consisting of hydrogen, —F, —Cl, -Et and ethynyl.
Embodiment 71. The compound of any one of embodiments 26 to 34 and 61 to 68, wherein R⁴ is selected from the group consisting of hydrogen, —F, —Cl, -Et and ethynyl.
Embodiment 72. The compound of any one of embodiments 26 to 34 and 61 to 68, wherein R⁴ is selected from the group consisting of—F, —Cl, -Et and ethynyl.
Embodiment 73. The compound of any one of embodiments 26 to 34 and 61 to 68, wherein R⁴ is selected from the group consisting of —F and —Cl.
Embodiment 74. The compound of any one of embodiments 26 to 34 and 61 to 68, wherein R⁴ is —F.
Embodiment 75. The compound of any one of embodiments 26 to 34 and 61 to 68, wherein R⁴ is -Et and ethynyl.
Embodiment 76. The compound of any one of embodiments 26 to 34 and 61 to 68, wherein R⁴ is ethynyl.
Embodiment 77. The compound of any one of embodiments 26 to 34 and 61 to 68, wherein R⁴ is chloro.
Embodiment 78. The compound of any one of embodiments 26 to 34 and 61 to 68, wherein R⁴ is hydrogen.
Embodiment 79. The compound of embodiment 1 or 2, wherein Ring A is isoquinolinyl substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl.
Embodiment 80. The compound of embodiment 79, wherein the isoquinolinyl is substituted with 1, 2 or 3 substituents independently selected from halo and —NH₂.
Embodiment 81. The compound of embodiment 79, wherein the isoquinolinyl is substituted with 1 or 2 substituents independently selected from —F, —C₁ and —NH₂.
Embodiment 82. The compound of any one of embodiments 79 to 81, wherein the isoquinolinyl is isoquinolinyl-1-yl.
Embodiment 83. The compound of any one of embodiments 1 to 10, wherein Ring A is selected from the group consisting of

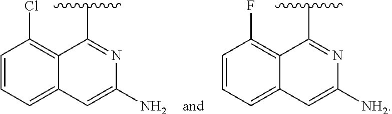 [SMILES: *c1nc(N)cc2cccc(Cl)c12 ; *c1nc(N)cc2cccc(F)c12]

Embodiment 84. The compound of any one of embodiments 1 to 10, wherein Ring A is

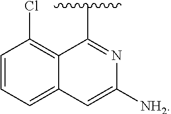 [SMILES: *c1nc(N)cc2cccc(Cl)c12]

Embodiment 85. The compound of any one of embodiments 1 to 10, wherein Ring A is

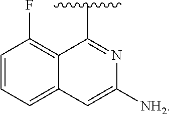 [SMILES: *c1nc(N)cc2cccc(F)c12]

Embodiment 86. The compound of embodiment 1 or 2, wherein the compound is of Formula III or Formula IV:

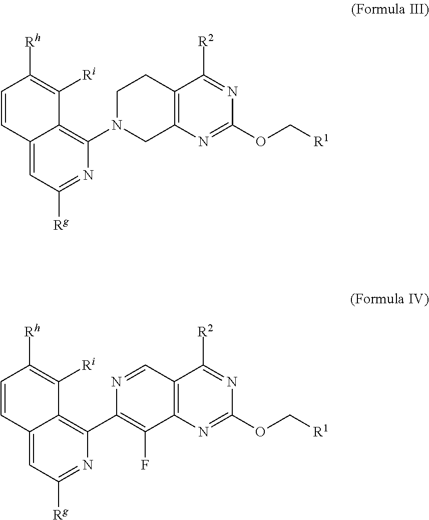

or a salt thereof; and/or an isotopologue thereof, wherein:
R^{h} is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl;
Rⁱ is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl; and
R^{g} is selected from the group consisting of hydrogen, halo, —NH₂, C₁-C₄ alkyl, C₁-C₄ alkoxy, C₁-C₄ haloalkyl, C₁-C₄ haloalkoxy and C₂-C₃ alkynyl.
Embodiment 87. The compound of embodiment 86, wherein the compound is of Formula III.
Embodiment 88. The compound of embodiment 86, wherein the compound is of Formula IV.
Embodiment 89. The compound of any one of embodiments 26 to 28, 30, 31, 35 and 86 to 88, wherein R^{h} and R are independently selected from the group consisting of hydrogen, —F, and —Cl.
Embodiment 90. The compound of any one of embodiments 26 to 28, 30, 31, 35 and 86 to 88, wherein R^{h} is selected from the group consisting of hydrogen and —F.
Embodiment 91. The compound of any one of embodiments 26 to 28, 30, 31, 35 and 86 to 88, wherein R^{h} is hydrogen.
Embodiment 92. The compound of any one of embodiments 26 to 28, 30, 31, 35 and 86 to 91, wherein Rⁱ is selected from the group consisting of —F and —Cl.
Embodiment 93. The compound of any one of embodiments 26 to 28, 30, 31, 35 and 86 to 91, wherein Rⁱ is —Cl.
Embodiment 94. The compound of any one of embodiments 26 to 28, 30, 31, 35 and 86 to 91, wherein Rⁱ is —F.
Embodiment 95. The compound of any one of embodiments 26 to 28, 30, 31, 35 and 86 to 94, wherein R⁹ is —NH₂.
Embodiment 96. The compound of any one of embodiments 1 to 10, wherein Ring A is phenyl substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl.
Embodiment 97. The compound of any one of embodiments 1 to 10, wherein Ring A is phenyl substituted with 0, 1, 2 or 3 substituents independently selected from halo, —OH, —NH₂, C₁-C₄ alkyl, C₁-C₄ alkenyl, C₁-C₄ alkoxy, C₁-C₄ haloalkyl, C₁-C₄ haloalkoxy and C₂-C₃ alkynyl.
Embodiment 98. The compound of embodiment 97, wherein the phenyl is substituted with 1, 2 or 3 substituents independently selected from halo, —NH₂, C₁-C₄ acyclic alkyl, C₃-C₄ cycloalkyl, C₁-C₄ alkenyl and C₁-C₄ haloalkyl.
Embodiment 99. The compound of embodiment 97, wherein the phenyl is substituted with 1, 2 or 3 substituents independently selected from —F, —Cl, -Me, -Et, cyclopropyl, cyclobutyl, prop-1-en-2-yl, —CHF₂ and —CF₃.
Embodiment 100. The compound of embodiment 97, wherein the phenyl is substituted with 1 or 2 substituents independently selected from —F, —Cl, cyclopropyl and —CF₃.
Embodiment 101. The compound of embodiment 97, wherein the phenyl is substituted with 2 substituents independently selected from —F, —Cl, cyclopropyl and —CF₃.
Embodiment 102. The compound of any one of embodiments 1 to 10, wherein Ring A is selected from the group consisting of:

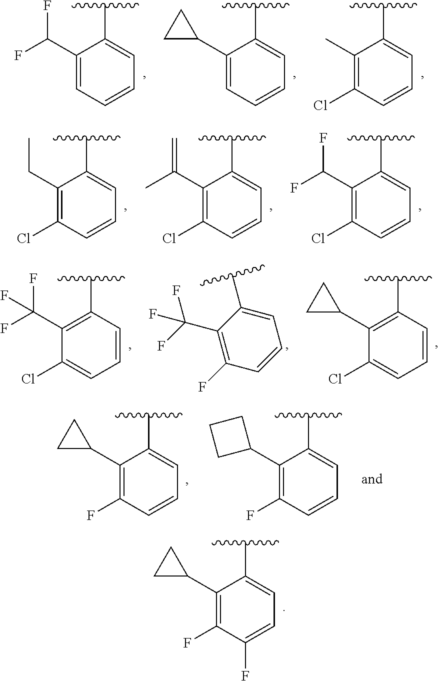

Embodiment 103. The compound of any one of embodiments 1 to 10, wherein Ring A is selected from the group consisting of:

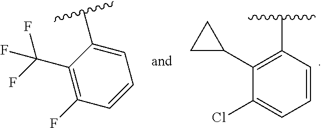 [SMILES: *c1cccc(F)c1C(F)(F)F ; *c1cccc(Cl)c1C1CC1]

Embodiment 104. The compound of any one of embodiments 1 to 10, wherein Ring A is

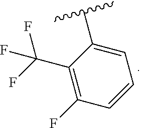 [SMILES: *c1cccc(F)c1C(F)(F)F]

Embodiment 105. The compound of any one of embodiments 1 to 10, wherein Ring A is

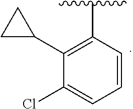 [SMILES: *c1cccc(Cl)c1C1CC1]

Embodiment 106. The compound of embodiment 1, wherein the compound is of Formula V or Formula VI:

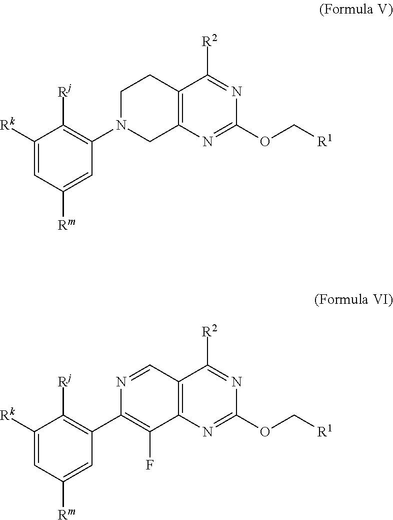

or a salt thereof, and/or an isotopologue thereof, wherein:
R^{j} is selected from the group consisting of hydrogen, halo, C₁-C₄ alkyl, C₁-C₄ alkoxy, C₁-C₄ haloalkyl, C₁-C₄ alkenyl, C₁-C₄ haloalkoxy and C₂-C₃ alkynyl;
R^{k} is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl;
R^{m} is selected from the group consisting of hydrogen, halo, —NH₂, C₁-C₄ alkyl, C₁-C₄ alkoxy, C₁-C₄ haloalkyl, C₁-C₄ haloalkoxy and C₂-C₃ alkynyl.
Embodiment 107. The compound of embodiment 1 or 2, wherein the compound is of Formula V or Formula VI:

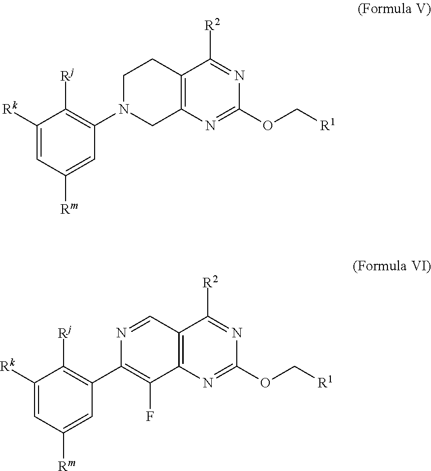

or a salt thereof, and/or an isotopologue thereof; wherein:
R^{j} is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl;
R^{k} is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl;
R^{m} is selected from the group consisting of hydrogen, halo, —NH₂, C₁-C₄ alkyl, C₁-C₄ alkoxy, C₁-C₄ haloalkyl, C₁-C₄ haloalkoxy and C₂-C₃ alkynyl.
Embodiment 108. The compound of embodiment 106 or 107, wherein the compound is of Formula V.
Embodiment 109. The compound of embodiment 106 or 107, wherein the compound is of Formula VI.
Embodiment 110. The compound of any one of embodiments 26, 28, 29, 33, 36 and 106 to 109, wherein R^{j} and R^{k} are independently selected from the group consisting of hydrogen, halo, C₁-C₄ acyclic alkyl, C₃-C₄ cycloalkyl, C₁-C₄ alkenyl and C₁-C₄ haloalkyl.
Embodiment 111. The compound of any one of embodiments 26 to 29, 33, 36 and 106 to 109, wherein R^{j} and R^{k} are independently selected from the group consisting of hydrogen, halo, C₃-C₄ cycloalkyl and C₁-C₄ haloalkyl.
Embodiment 112. The compound of any one of embodiments 26, 28, 29, 33, 36 and 106 to 109, wherein R^{j} and R^{k} are independently selected from the group consisting of hydrogen, —F, —Cl, -Me, -Et, cyclopropyl, cyclobutyl, prop-1-en-2-yl, —CHF₂ and —CF₃.
Embodiment 113. The compound of any one of embodiments 26 to 29, 33, 36 and 106 to 109, wherein R^{j} and R^{k} are independently selected from the group consisting of —F, —Cl, cyclopropyl and —CF₃.
Embodiment 114. The compound of any one of embodiments 26 to 29, 33, 36 and 106 to 109, wherein R^{j} and R^{k} are independently selected from the group consisting of hydrogen, —F, —C₁ and —CHF₂.
Embodiment 115. The compound of any one of embodiments 26, 28, 29, 33, 36 and 106 to 109, wherein R^{j} is selected from the group consisting of C₁-C₄ acyclic alkyl, C₃-C₄ cycloalkyl, C₁-C₄ alkenyl and C₁-C₄ haloalkyl.
Embodiment 116. The compound of any one of embodiments 26 to 29, 33, 36 and 106 to 109, wherein R^{j} is selected from the group consisting of C₃-C₄ cycloalkyl and C₁-C₄ haloalkyl.
Embodiment 117. The compound of any one of embodiments 26, 28, 29, 33, 36 and 106 to 109, wherein R^{j} is selected from the group consisting of -Me, -Et, cyclopropyl, cyclobutyl, prop-1-en-2-yl, —CHF₂ and —CF₃.
Embodiment 118. The compound of any one of embodiments 26 to 29, 33, 36 and 106 to 109, wherein R^{j} is selected from the group consisting of cyclopropyl and —CF₃.
Embodiment 119. The compound of any one of embodiments 26 to 29, 33, 36 and 106 to 109, wherein R^{j} is cyclopropyl.
Embodiment 120. The compound of any one of embodiments 26 to 29, 33, 36 and 106 to 109, wherein R^{j} is —CF₃.
Embodiment 121. The compound of any one of embodiments 26 to 29, 33, 36 and 106 to 109, wherein R^{j} is difluoromethyl.
Embodiment 122. The compound of any one of embodiments 26 to 29, 33, 36 and 106 to 121, wherein R^{k} is selected from the group consisting of hydrogen and halo.
Embodiment 123. The compound of any one of embodiments 26 to 29, 33, 36 and 106 to 121, wherein R^{k} is selected from the group consisting of hydrogen, —F and —Cl.
Embodiment 124. The compound of any one of embodiments 26 to 29, 33, 36 and 106 to 121, wherein R^{k} is —F.
Embodiment 125. The compound of any one of embodiments 26 to 29, 33, 36 and 106 to 121, wherein R^{k} is —C₁.
Embodiment 126. The compound of any one of embodiments 26 to 29, 33, 36 and 106 to 121, wherein R^{k} is hydrogen.
Embodiment 127. The compound of any one of embodiments 26 to 29, 33, 36 and 106 to 126, wherein R^{m} is hydrogen.
Embodiment 128. The compound of any one of embodiments 1 to 10, wherein Ring A is pyridinyl substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl.
Embodiment 129. The compound of embodiment 128, wherein the pyridinyl is substituted with 1, 2 or 3 substituents independently selected from —NH₂, C₁-C₄ acyclic alkyl and C₁-C₄ haloalkyl.
Embodiment 130. The compound of embodiment 128, wherein the pyridinyl is substituted with 1, 2 or 3 substituents independently selected from —NH₂, -Me and —CF₃.
Embodiment 131. The compound of any one of embodiments 128 to 130, wherein the pyridinyl is pyridin-2-yl.
Embodiment 132. The compound of any one of embodiments 1 to 10, wherein Ring A is

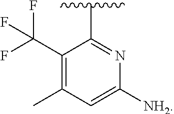 [SMILES: *c1nc(N)cc(C)c1C(F)(F)F]

Embodiment 133. The compound of any one of embodiments 1 to 10, wherein the compound is of Formula VII, Formula VIII or Formula IX:

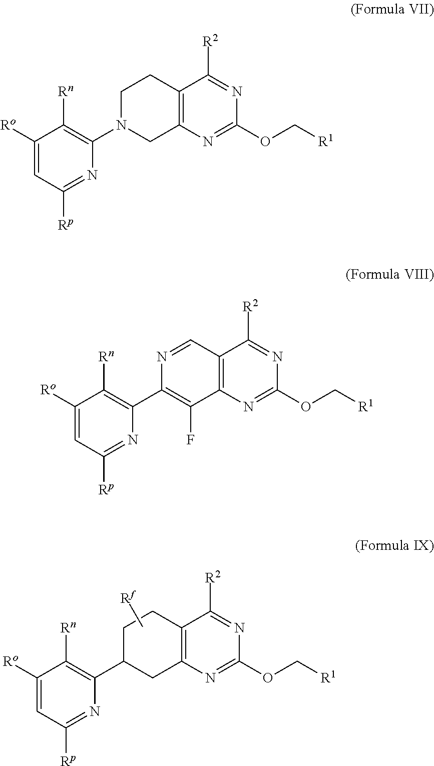

or a salt thereof; and/or an isotopologue thereof, wherein:
Rⁿ is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl;
R^{o} is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl;
R^{p} is selected from the group consisting of hydrogen, halo, —NH₂, C₁-C₄ alkyl, C₁-C₄ alkoxy, C₁-C₄ haloalkyl, C₁-C₄ haloalkoxy and C₂-C₃ alkynyl.
Embodiment 134. The compound of embodiment 133, wherein the compound is of Formula VII.
Embodiment 135. The compound of embodiment 133, wherein the compound is of Formula VIII.
Embodiment 136. The compound of embodiment 133, wherein the compound is of Formula IX.
Embodiment 137. The compound of embodiment 133, wherein the compound is of Formula IXa:

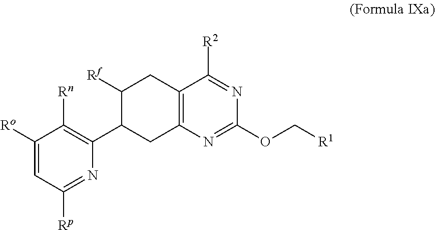

or a salt thereof; and/or an isotopologue thereof.
Embodiment 138. The compound of any one of embodiments 26, 27, 29, 30, 32, 37 and 133 to 137, wherein Rⁿ and R^{o} are independently selected from the group consisting of hydrogen, methyl and trifluoromethyl.
Embodiment 139. The compound of any one of embodiments 26, 27, 29, 30, 32, 37 and 133 to 137, wherein Rⁿ is trifluoromethyl.
Embodiment 140. The compound of any one of embodiments 26, 27, 29, 30, 32, 37, 133 to 137 and 139, wherein R^{o} is methyl.
Embodiment 141. The compound of any one of embodiments 26, 27, 29, 30, 32, 37, 133 to 137 and 139, wherein R^{o} is fluoro.
Embodiment 142. The compound of any one of embodiments 26, 27, 29, 30, 32, 37 and 133 to 141, wherein R^{P} is —NH₂.
Embodiment 143. The compound of any one of embodiments 1, 2, 6, 7, 10, 11 to 60 and 133 to 142, wherein R is methyl.
Embodiment 144. The compound of any one of embodiments 1 to 10, wherein Ring A is 1-H-indazolyl substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl.
Embodiment 145. The compound of embodiment 144, wherein the 1-H-indazolyl is substituted with 1 or 2 substituents independently selected from halo and acyclic C₁-C₄ alkyl.
Embodiment 146. The compound of embodiment 144, wherein the 1-H-indazolyl is substituted with 1 or 2 substituents independently selected from —F, —Cl and -Me.
Embodiment 147. The compound of any one of embodiments 144 to 146, wherein the 1-H-indazolyl is 1-H-indazol-3-yl.
Embodiment 148. The compound of any one of embodiments 144 to 146, wherein the 1-H-indazolyl is 1-H-indazol-4-yl.
Embodiment 149. The compound of any one of embodiments 144 to 146 wherein Ring A is selected from

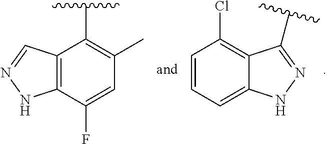 [SMILES: *c1c(C)cc(F)c2[nH]ncc12 ; *c1n[nH]c2cccc(Cl)c12]

Embodiment 150. The compound of any one of embodiments 144 to 146 wherein Ring A is

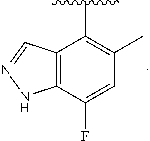 [SMILES: *c1c(C)cc(F)c2[nH]ncc12]

Embodiment 151. The compound of any one of embodiments 144 to 146 wherein Ring A is

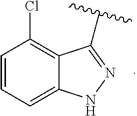 [SMILES: *c1n[nH]c2cccc(Cl)c12]

Embodiment 152. The compound of any one of embodiments 1 to 151, wherein R^{d} is selected from the group consisting of H and —F.
Embodiment 153. The compound of any one of embodiments 1 to 151, wherein R^{d} is —OH.
Embodiment 154. The compound of any one of embodiments 1 to 151, wherein R^{d} is H.
Embodiment 155. The compound of any one of embodiments 1 to 151, wherein R^{d} is F.
Embodiment 156. The compound of any one of embodiments 1 to 155, wherein R^{2c} is selected from the group consisting of

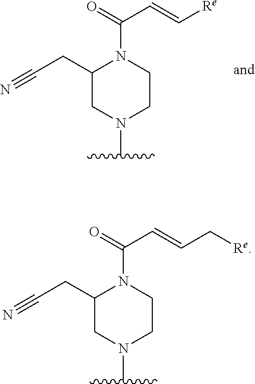

Embodiment 157. The compound of any one of embodiments 1 to 155, wherein R^{2c} is

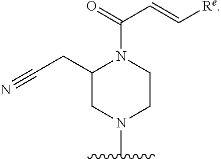

Embodiment 158. The compound of any one of embodiments 1 to 155, wherein R^{2c} is

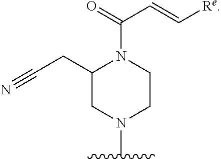

Embodiment 159. The compound of any one of embodiments 1 to 155, wherein R^{2c} is

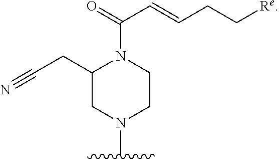

Embodiment 160. The compound of any one of embodiments 1 to 159, wherein R^{e} is R.
Embodiment 161. The compound of any one of embodiments 1 to 160, wherein R^{e1} is a 4-7 membered monocyclic heterocycle containing a nitrogen or an oxygen atom as the only heteroatom, wherein the monocyclic heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 162. The compound of any one of embodiments 1 to 160, wherein R is a 4-7 membered monocyclic heterocycle containing a nitrogen atom as the only heteroatom or containing one nitrogen atom and one oxygen atom, wherein the monocyclic heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 163. The compound of embodiment 161 or 162, wherein the monocyclic heterocycle of R is substituted with 0 or 1 instances of C₁-C₄ alkyl.
Embodiment 164. The compound of embodiment 161 or 162, wherein the monocyclic heterocycle of R is substituted with 0 or 1 instances of -Me, -ⁱPr or cyclopropyl.
Embodiment 165. The compound of embodiment 161 or 162, wherein the monocyclic heterocycle of R is substituted with 0 or 1 instances of -ⁱPr.
Embodiment 166. The compound of embodiment 161 or 162, wherein the monocyclic heterocycle of R is substituted with 0 or 1 instances of cyclopropyl.
Embodiment 167. The compound of embodiment 161 or 162, wherein the monocyclic heterocycle of R^{W} is substituted with 0 or 1 instance of methyl.
Embodiment 168. The compound of embodiment 161 or 162, wherein R^{e1} is selected from the group consisting of azetidinyl and oxetanyl, each substituted with 0 or 1 instances of C₁-C₄ alkyl.
Embodiment 169. The compound of embodiment 161 or 162, wherein R^{e1} is selected from the group consisting of azetidinyl and oxetanyl, each substituted with 0 or 1 substituents independently selected from -Me, -ⁱPr or cyclopropyl.
Embodiment 170. The compound of embodiment 162, wherein R is selected from azetidinyl, pyrrolidinyl and morpholinyl substituted with 0 or 1 instance of -Me.
Embodiment 171. The compound of embodiment 161 or 162, wherein R is azetidinyl substituted with 0 or 1 instances of C₁-C₄ alkyl.
Embodiment 172. The compound of embodiment 161 or 162, wherein R is azetidinyl substituted with 0 or 1 substituents independently selected from -Me, -ⁱPr or cyclopropyl.
Embodiment 173. The compound of embodiment 161 or 162, wherein R is azetidinyl substituted with 0 or 1 instance of -Me.
Embodiment 174. The compound of embodiment 161 or 162, wherein R is azetidinyl substituted with 0 or 1 instance of cyclopropyl.
Embodiment 175. The compound of embodiment 161 or 162, wherein R is azetidinyl substituted with 0 or 1 instance of -ⁱPr.
Embodiment 176. The compound of embodiment 161, wherein R is oxetanyl substituted with 0 or 1 instances of C₁-C₄ alkyl.
Embodiment 177. The compound of embodiment 161, wherein R is oxetanyl substituted with 0 or 1 instance of -Me.
Embodiment 178. The compound of embodiment 161 or 162, wherein R is N-methyl azetidinyl.
Embodiment 179. The compound of any one of embodiments 161 to 178, wherein the attachment point for the monocyclic heterocycle is on a carbon atom.
Embodiment 180. The compound of embodiment 161, wherein R is selected from the group consisting of:

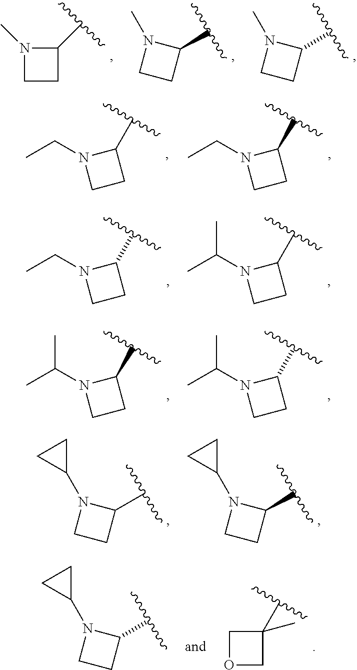

Embodiment 181. The compound of embodiment 161, wherein R is selected from the group consisting of:

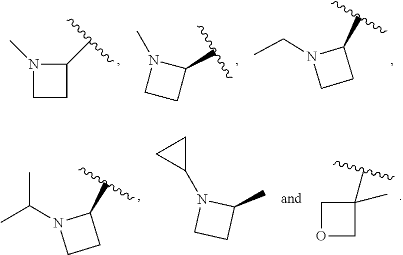

Embodiment 182. The compound of embodiment 161, wherein R is selected from the group consisting of:

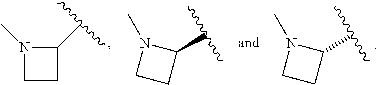

Embodiment 183. The compound of embodiment 161 wherein R^{e1} is

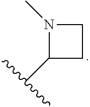 [SMILES: *C1CCN1C]

Embodiment 184. The compound of embodiment 161, wherein R^{e1} is

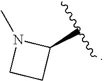 [SMILES: *[C@H]1CCN1C]

Embodiment 185. The compound of embodiment 161, wherein R^{e1} is

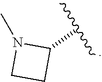 [SMILES: *[C@@H]1CCN1C]

Embodiment 186. The compound of any one of embodiments 161 to 185, wherein R^{2c} is

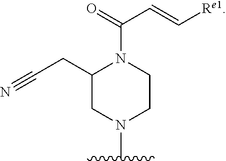

Embodiment 187. The compound of any one of embodiments 1 to 160, wherein R is a 4-10 membered heterocycle containing a nitrogen atom and one or two additional heteroatoms selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 188. The compound of any one of embodiments 1 to 160, wherein R^{e1} is a 4-10 membered heterocycle containing a nitrogen atom and one or two additional heteroatoms selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 189. The compound of any one of embodiments 1 to 160, wherein R is a 4-10 membered heterocycle containing a nitrogen atom and one or two additional heteroatoms selected from oxygen and sulfur, including sulfur dioxide, selected from the group consisting of a 4-8 member monocyclic heterocycle, a 6-10 member fused bicyclic heterocycle, a 6-10 member bridged heterocycle and a 6-10 member spiro heterocycle, each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 190. The compound of any one of embodiments 1 to 160, wherein R is a 4-10 membered heterocycle containing a nitrogen atom and one or two additional heteroatoms selected from oxygen and sulfur, including sulfur dioxide, selected from the group consisting of a 4-8 member monocyclic heterocycle, a 6-10 member fused bicyclic heterocycle, a 6-10 member bridged heterocycle and a 6-10 member spiro heterocycle, each substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 191. The compound of any one of embodiments 1 to 160, wherein R is a 4-8 member monocyclic heterocycle substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 192. The compound of any one of embodiments 1 to 160, wherein R is a 4-8 member monocyclic heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 193. The compound of any one of embodiments 1 to 160, wherein R is a 6-10 member fused bicyclic heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 194. The compound of any one of embodiments 1 to 160, wherein R is a 6-10 member bridged heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 195. The compound of any one of embodiments 1 to 160, wherein R^{e1} is a 6-10 member spiro heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 196. The compound of any one of embodiments 1 to 160, wherein R^{e1} is selected from azetidine, pyrrolidine, isothiazolidine 1,1-dioxide, 3-azabicyclo[3.1.0]hexane, piperidine, piperazine, 2-oxabicyclo[4.1.0]heptane, 3-oxa-6-azabicyclo[3.2.0]heptane, hexahydro-1H-furo[3,4-c]pyrrole, hexahydro-1H-furo[3,4-b]pyrrole, 2-azabicyclo[2.1.1]hexane, morpholine, 2-oxa-5-azabicyclo[4.1.0]heptane, 1, 4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 7-oxa-2-azabicyclo[3.3.1]nonane, 8-oxa-3-azabicyclo[3.2.1]octane, 9-oxa-3-azabicyclo[3.3.1]nonane, 9-oxa-3-azabicyclo[3.3.1]nonane, 2-oxa-6-azaspiro[3.3]heptane, 3-oxa-6-azabicyclo[3.1.1]heptane, 6-oxa-3-azabicyclo[3.1.1]heptane, 2-oxa-5-azabicyclo[2.2.2]octane, 5,6,7,8-tetrahydro-[1,2,4]triazolo[4,3-a]pyrazine, 5,6,7,8-tetrahydro-[1,2,4]triazolo[1,5-a]pyrazine, 4,5,6,7-tetrahydropyrazolo[1,5-a]pyrazine, 5,6,7,8-tetrahydroimidazo[1,2-a]pyrazine, thiomorpholine, thiomorpholine 1,1-dioxide, 4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 197. The compound of any one of embodiments 1 to 160, wherein R^{e1} is selected from azetidine, pyrrolidine, 2-azabicyclo[2.1.1]hexane, morpholine, 2-oxa-5-azabicyclo[4.1.0]heptane, 1, 4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 7-oxa-2-azabicyclo[3.3.1]nonane, 8-oxa-3-azabicyclo[3.2.1]octane, 9-oxa-3-azabicyclo[3.3.1]nonane, 9-oxa-3-azabicyclo[3.3.1]nonane, 2-oxa-6-azaspiro[3.3]heptane, 3-oxa-6-azabicyclo[3.1.1]heptane, 6-oxa-3-azabicyclo[3.1.1]heptane, thiomorpholine, thiomorpholine 1,1-dioxide, 4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 198. The compound of any one of embodiments 1 to 160, wherein R^{e1} is selected from azetidine, pyrrolidine, isothiazolidine 1,1-dioxide, 3-azabicyclo[3.1.0]hexane, piperidine, piperazine, 2-oxabicyclo[4.1.0]heptane, 3-oxa-6-azabicyclo[3.2.0]heptane, hexahydro-1H-furo[3,4-c]pyrrole, hexahydro-1H-furo[3,4-b]pyrrole, morpholine, 2-oxa-5-azabicyclo[4.1.0]heptane, 1, 4-oxazepane, 2-oxa-5-azabicyclo[2.2.1]heptane, 3,9-dioxa-7-azabicyclo[3.3.1]nonane, 3-oxa-8-azabicyclo[3.2.1]octane, 8-oxa-3-azabicyclo[3.2.1]octane, 2-oxa-6-azaspiro[3.3]heptane, 3-oxa-6-azabicyclo[3.1.1]heptane, 6-oxa-3-azabicyclo[3.1.1]heptane, 2-oxa-5-azabicyclo[2.2.2]octane, 5,6,7,8-tetrahydro-[1,2,4]triazolo[4,3-a]pyrazine, 5,6,7,8-tetrahydro-[1,2,4]triazolo[1,5-a]pyrazine, 4,5,6,7-tetrahydropyrazolo[1,5-a]pyrazine, 5,6,7,8-tetrahydroimidazo[1,2-a]pyrazine, thiomorpholine and thiomorpholine 1,1-dioxide, each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 199. The compound of any one of embodiments 1 to 160, wherein R^{e1} is selected from azetidine, pyrrolidine, isothiazolidine 1,1-dioxide, 3-azabicyclo[3.1.0]hexane, piperidine, piperazine, 2-oxabicyclo[4.1.0]heptane, 3-oxa-6-azabicyclo[3.2.0]heptane, hexahydro-1H-furo[3,4-c]pyrrole, hexahydro-1H-furo[3,4-b]pyrrole, morpholine, 2-oxa-5-azabicyclo[4.1.0]heptane, 1, 4-oxazepane, 2-oxa-5-azabicyclo[2.2.1]heptane, 3,9-dioxa-7-azabicyclo[3.3.1]nonane, 3-oxa-8-azabicyclo[3.2.1]octane, 8-oxa-3-azabicyclo[3.2.1]octane, 2-oxa-6-azaspiro[3.3]heptane, 3-oxa-6-azabicyclo[3.1.1]heptane, 6-oxa-3-azabicyclo[3.1.1]heptane, 2-oxa-5-azabicyclo[2.2.2]octane, 5,6,7,8-tetrahydro-[1,2,4]triazolo[4,3-a]pyrazine, 5,6,7,8-tetrahydro-[1,2,4]triazolo[1,5-a]pyrazine, 4,5,6,7-tetrahydropyrazolo[1,5-a]pyrazine, 5,6,7,8-tetrahydroimidazo[1,2-a]pyrazine, thiomorpholine and thiomorpholine 1,1-dioxide, each substituted with 0, 1 or 2 substituents independently selected from —F, -Me, —OMe, -OEt, —OCHF₂, —C(═O)Me and oxetanyl.
Embodiment 200. The compound of any one of embodiments 1 to 160, wherein R^{e1} is selected from azetidine, pyrrolidine, piperidine, hexahydro-1H-furo[3,4-c]pyrrole, morpholine, 1, 4-oxazepane, 2-oxa-5-azabicyclo[2.2.1]heptane, 3-oxa-8-azabicyclo[3.2.1]octane, 8-oxa-3-azabicyclo[3.2.1]octane, 3-oxa-6-azabicyclo[3.1.1]heptane, 6-oxa-3-azabicyclo[3.1.1]heptane, 2-oxa-5-azabicyclo[2.2.2]octane and thiomorpholine, each substituted with 0, 1 or 2 substituents independently selected from —F, -Me, —OMe and -OEt.
Embodiment 201. The compound of embodiment 196, wherein R is selected from hexahydro-1H-furo[3,4-c]pyrrole, morpholine, 1, 4-oxazepane, 2-oxa-5-azabicyclo[2.2.1]heptane, 3-oxa-8-azabicyclo[3.2.1]octane, 8-oxa-3-azabicyclo[3.2.1]octane, 3-oxa-6-azabicyclo[3.1.1]heptane, 6-oxa-3-azabicyclo[3.1.1]heptane, 2-oxa-5-azabicyclo[2.2.2]octane and thiomorpholine, each unsubstituted.
Embodiment 202. The compound of any one of embodiments 1 to 160, wherein R^{e1} is azetidine substituted with 0, 1 or 2 substituents independently selected from —F and —OMe and -OEt.
Embodiment 203. The compound of any one of embodiments 1 to 160, wherein R^{e1} is pyrrolidine substituted with 0, 1 or 2 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 204. The compound of any one of embodiments 1 to 160, wherein R^{e1} is pyrrolidine substituted with 0, 1 or 2 substituents independently selected from —F, —OMe, -OEt and —OCHF₂.
Embodiment 205. The compound of any one of embodiments 1 to 160, wherein R^{e1} is piperidine substituted with 0, 1 or 2 instances of —F.
Embodiment 206. The compound of any one of embodiments 1 to 160, wherein R^{e1} is unsubstituted hexahydro-1H-furo[3,4-c]pyrrole.
Embodiment 207. The compound of any one of embodiments 1 to 160, wherein R^{e1} is unsubstituted 1, 4-oxazepane.
Embodiment 208. The compound of any one of embodiments 1 to 160, wherein R^{e1} is unsubstituted 2-oxa-5-azabicyclo[2.2.1]heptane.
Embodiment 209. The compound of any one of embodiments 1 to 160, wherein R^{e1} is unsubstituted 3-oxa-8-azabicyclo[3.2.1]octane.
Embodiment 210. The compound of any one of embodiments 1 to 160, wherein R^{e1} is unsubstituted 8-oxa-3-azabicyclo[3.2.1]octane.
Embodiment 211. The compound of any one of embodiments 1 to 160, wherein R^{e1} is unsubstituted 3-oxa-6-azabicyclo[3.1.1]heptane.
Embodiment 212. The compound of any one of embodiments 1 to 160, wherein R^{e1} is unsubstituted 6-oxa-3-azabicyclo[3.1.1]heptane.
Embodiment 213. The compound of any one of embodiments 1 to 160, wherein R^{e1} is unsubstituted 2-oxa-5-azabicyclo[2.2.2]octane.
Embodiment 214. The compound of any one of embodiments 1 to 160, wherein R is unsubstituted thiomorpholine.
Embodiment 215. The compound of any one of embodiments 1 to 160, wherein R^{e1} is morpholine substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 216. The compound of any one of embodiments 1 to 160, wherein R^{e1} is morpholine substituted with 0, 1 or 2 instances of -Me.
Embodiment 217. The compound of any one of embodiments 187 to 216, wherein the attachment point for R is the nitrogen atom of the heterocycle.
Embodiment 218. The compound of any one of embodiments 1 to 160, wherein the R is selected from the group consisting of:

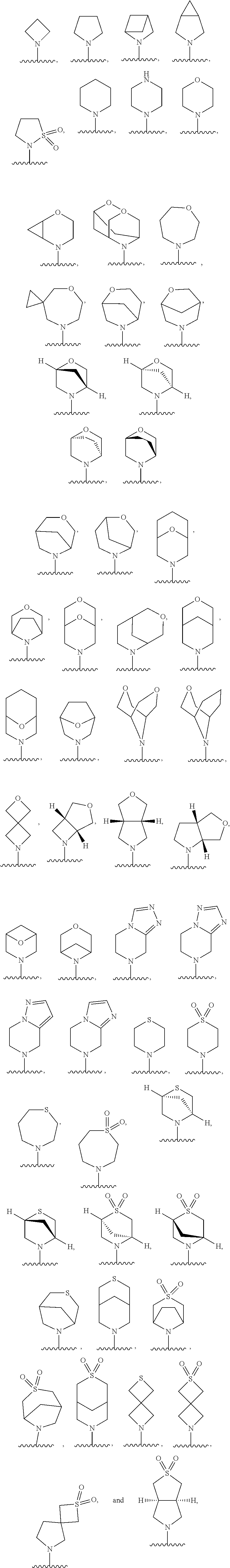

each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 219. The compound of any one of embodiments 1 to 160, wherein the R is selected from the group consisting of:

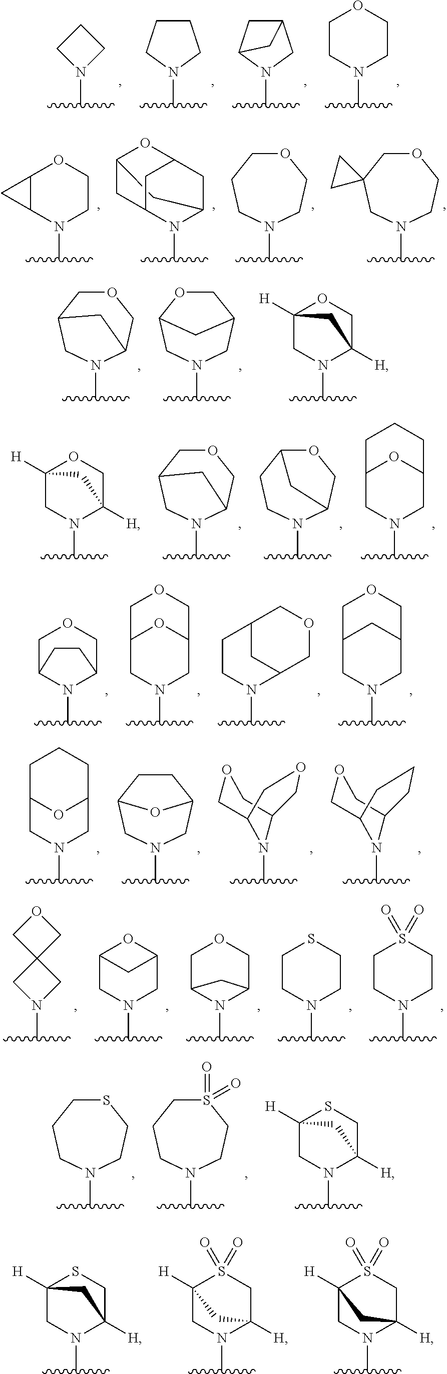

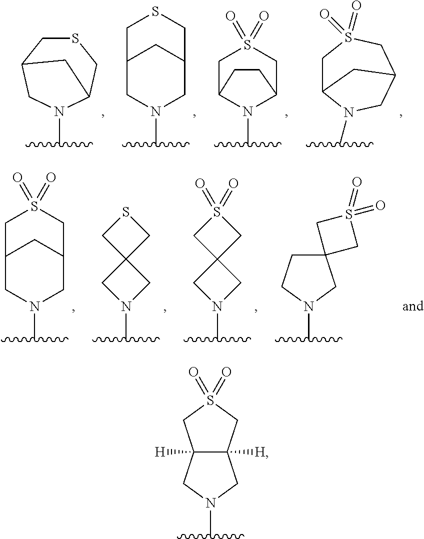

each substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 220. The compound of embodiment 217, wherein the R is selected from the group consisting of:

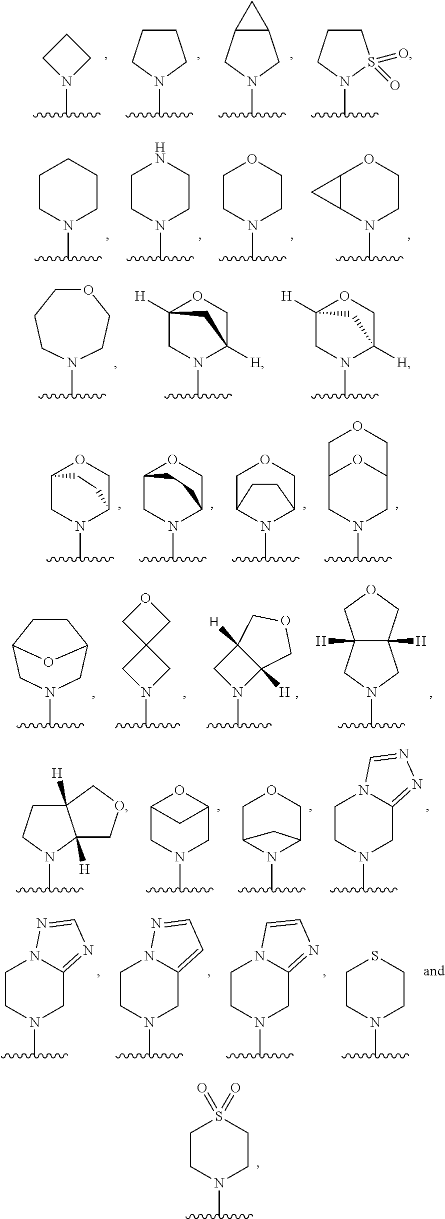

each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 221. The compound of any one of embodiments 1 to 160, wherein R^{e1} is

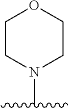 [SMILES: *N1CCOCC1]

substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 222. The compound of any one of embodiments 187 to 221, wherein the 4-10 membered heterocycle of R^{e1} is substituted with 0, 1, 2, 3 or 4 substituents independently selected from —F, -Me, -Et, —OH, —OMe, —CF₃, —OCF₃, —CH₂OCH₃, —CH₂CH₂OCH₃, —CH₂CH₂OCH₂CH₂OCH₃, —CH₂N(CH₃)₂, —CH₂CH₂N(CH₃)₂.
Embodiment 223. The compound of any one of embodiments 187 to 221, wherein the 4-10 membered heterocycle of R^{e1} is substituted with 0, 1 or 2 substituents independently selected from —F, -Me, —OMe, -OEt, —OCHF₂, —C(═O)Me and oxetanyl.
Embodiment 224. The compound of any one of embodiments 187 to 221, wherein the 4-10 membered heterocycle of R^{e1} is substituted with 0, 1 or 2 substituents independently selected from fluoro, methyl, ethyl, hydroxy, methoxy, trifluoromethyl, trifluoromethoxy, —CH₂OCH₃, —CH₂CH₂OCH₃, —CH₂CH₂OCH₂CH₂OCH₃, —CH₂N(CH₃)₂, —CH₂CH₂N(CH₃)₂.
Embodiment 225. The compound of any one of embodiments 187 to 221, wherein the 4-10 membered heterocycle of R^{e1} is substituted with 0, 1 or 2 substituents independently selected from fluoro and methyl.
Embodiment 226. The compound of any one of embodiments 1 to 160, wherein R^{e} is selected from

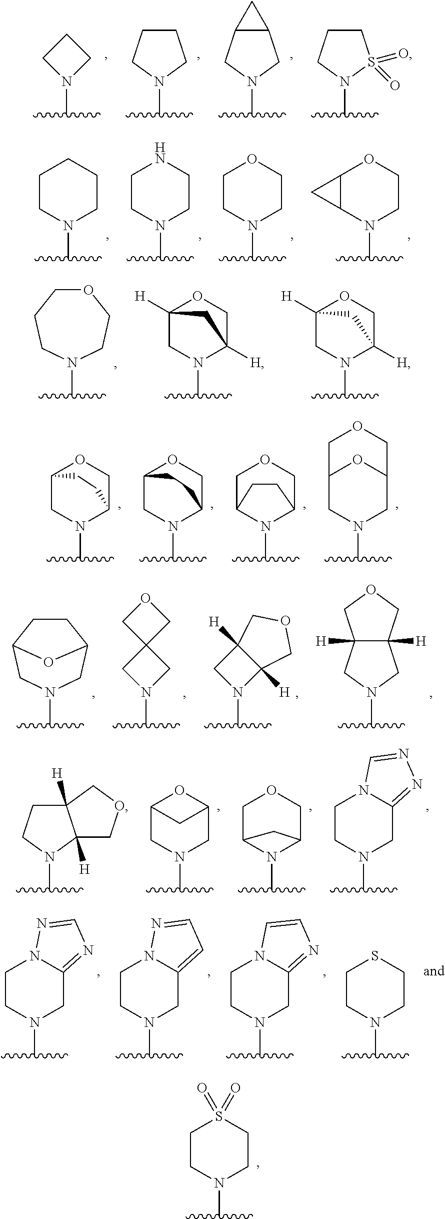

each substituted with 0, 1 or 2 substituents independently selected from —F, -Me, —OMe, -OEt, —OCHF₂, —C(═O)Me and oxetanyl.
Embodiment 227. The compound of any one of embodiments 1 to 160, wherein R^{e1} is selected from

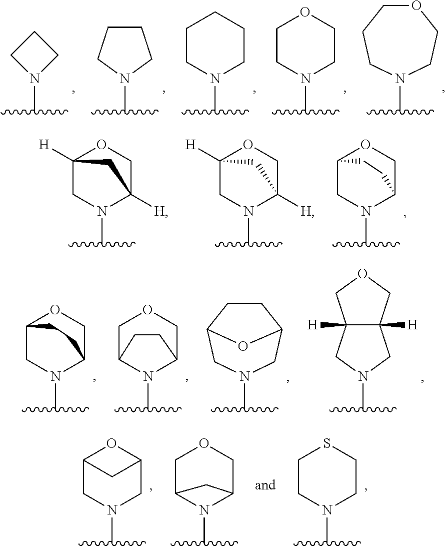

each substituted with 0.1 or 2 substituents independently selected from —F, -Me, —OMe and -OEt.
Embodiment 228. The compound of any one of embodiments 187 to 221, wherein the 4-10 membered heterocycle of R^{e1} is unsubstituted.
Embodiment 229. The compound of any one of embodiments 1 to 160, wherein R^{e1} is selected from,

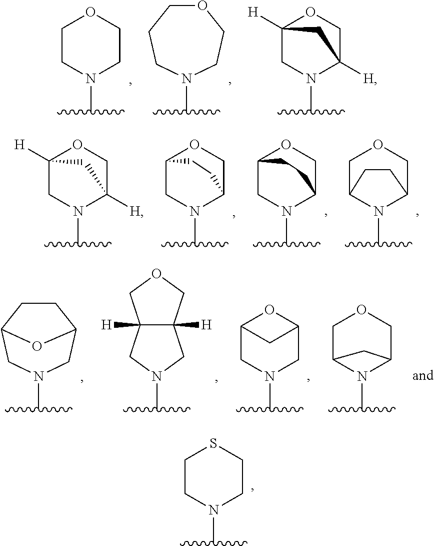

each unsubstituted.
Embodiment 230. The compound of any one of embodiments 1 to 160, wherein R^{e1} is

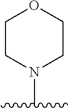 [SMILES: *N1CCOCC1]

substituted with 0, 1 or 2 instances of -Me.
Embodiment 231. The compound of any one of embodiments 1 to 160, wherein R^{e1} is

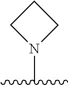 [SMILES: *N1CCC1]

substituted with 0, 1 or 2 substituents independently selected from —F and —OMe and -OEt.
Embodiment 232. The compound of any one of embodiments 1 to 160, wherein R^{e1} is

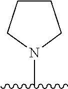 [SMILES: *N1CCCC1]

substituted with 0, 1 or 2 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.
Embodiment 233. The compound of any one of embodiments 1 to 160, wherein R^{e1} is

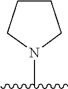 [SMILES: *N1CCCC1]

substituted with 0, 1 or 2 substituents independently selected from —F, —OMe, -OEt and —OCHF₂.
Embodiment 234. The compound of any one of embodiments 1 to 160, wherein R^{e1} is

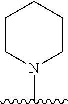 [SMILES: *N1CCCCC1]

substituted with 0, 1 or 2 instances of —F.
Embodiment 235. The compound of any one of embodiments 1 to 160, wherein R^{e1} is selected from the group consisting of

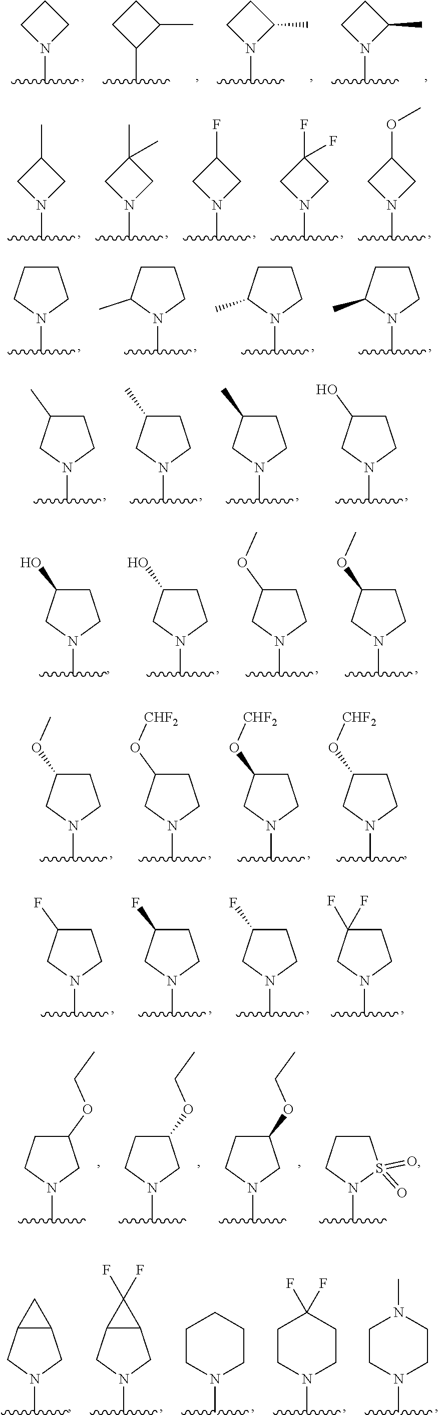

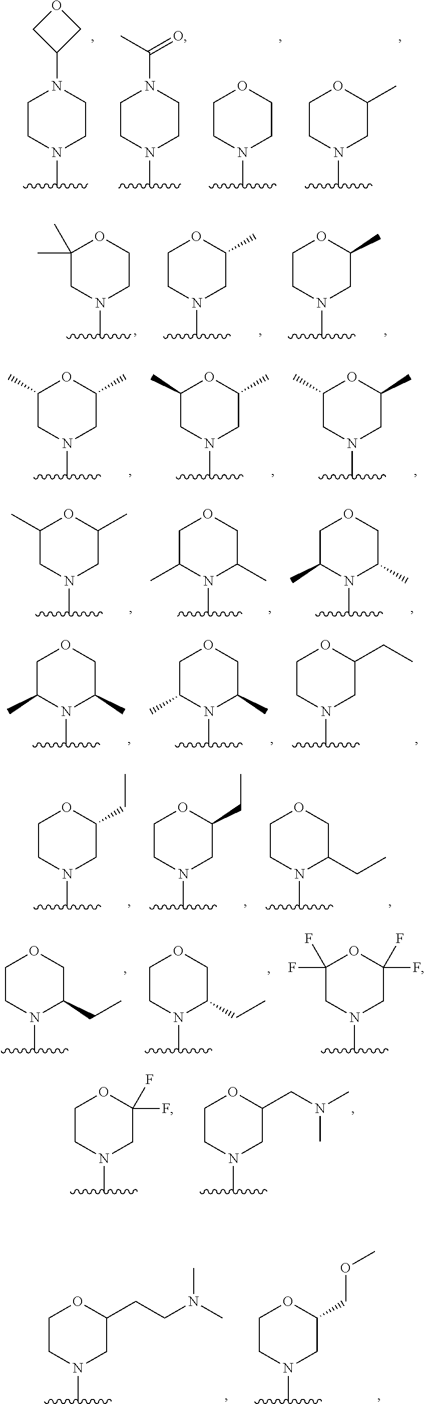

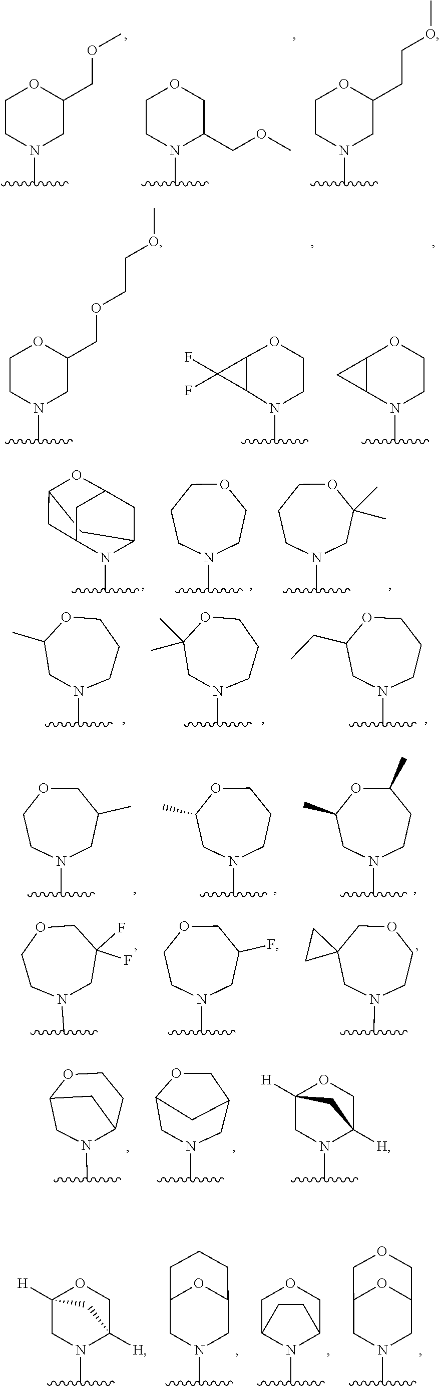

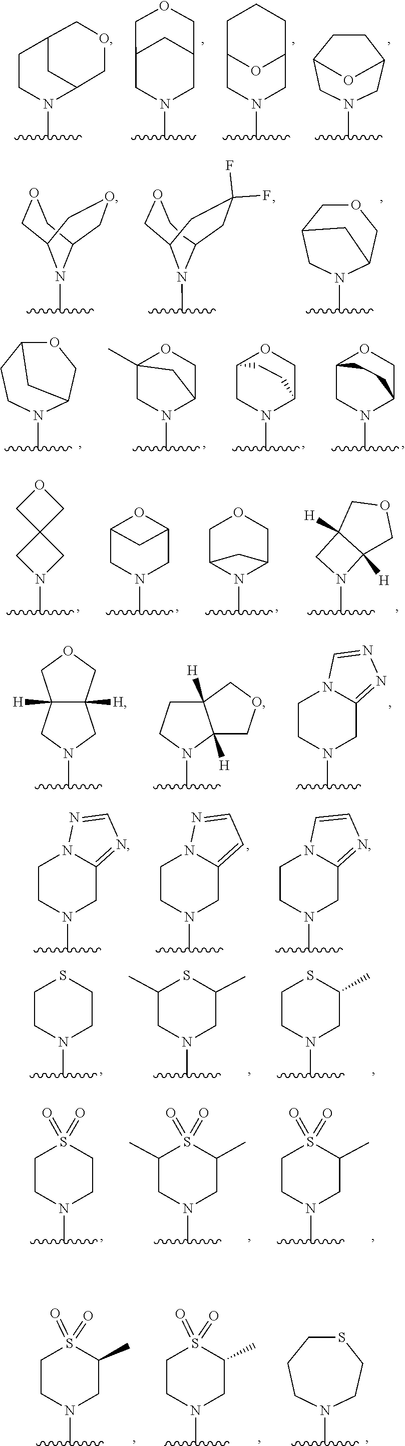

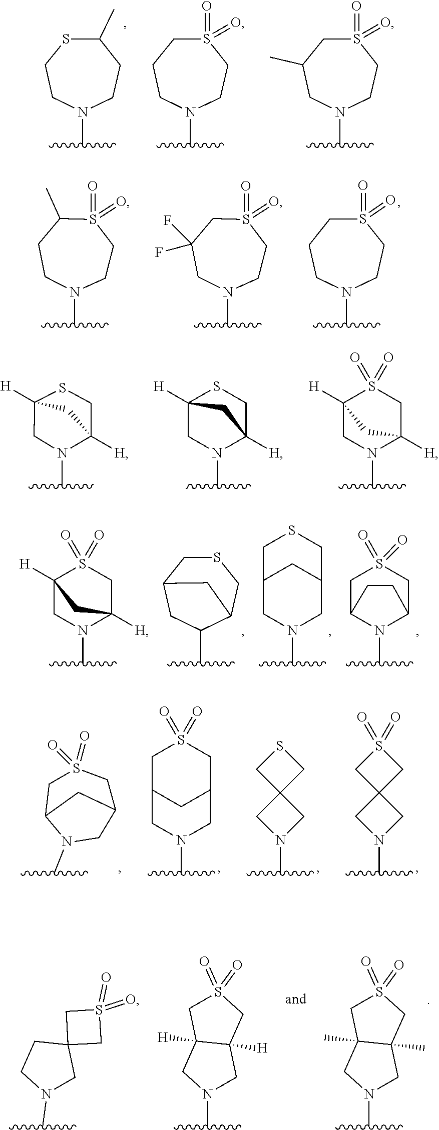

Embodiment 236. The compound of any one of embodiments 1 to 160, wherein R^{e1} is selected from the group consisting of

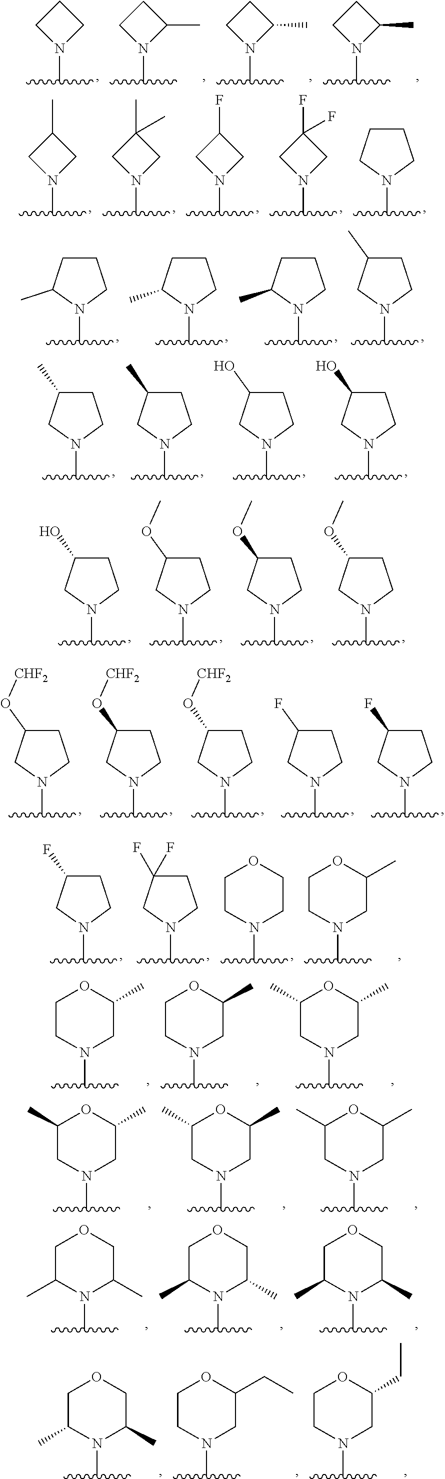

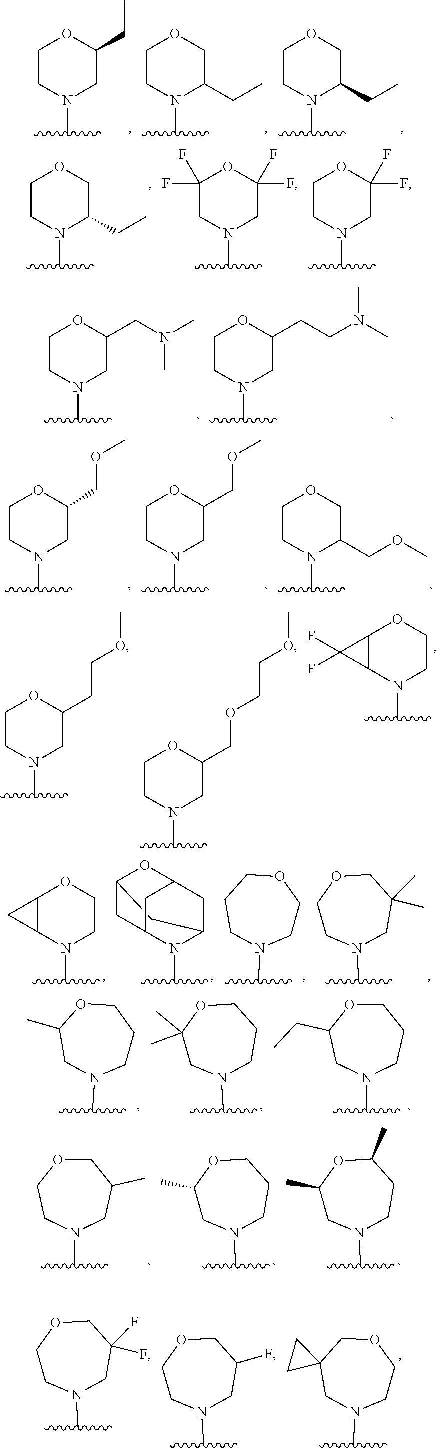

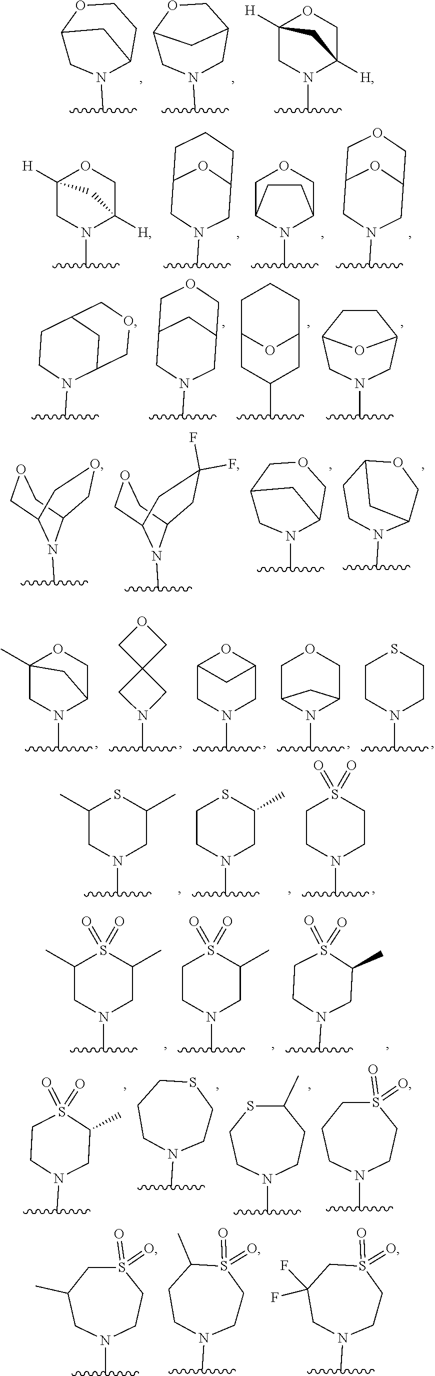

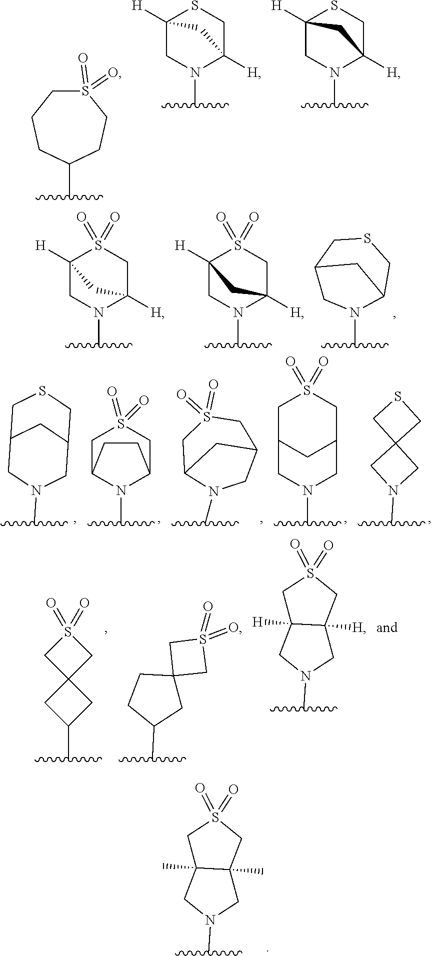

Embodiment 237. The compound of any one of embodiments 1 to 160, wherein R^{e1} is selected from the group consisting of:

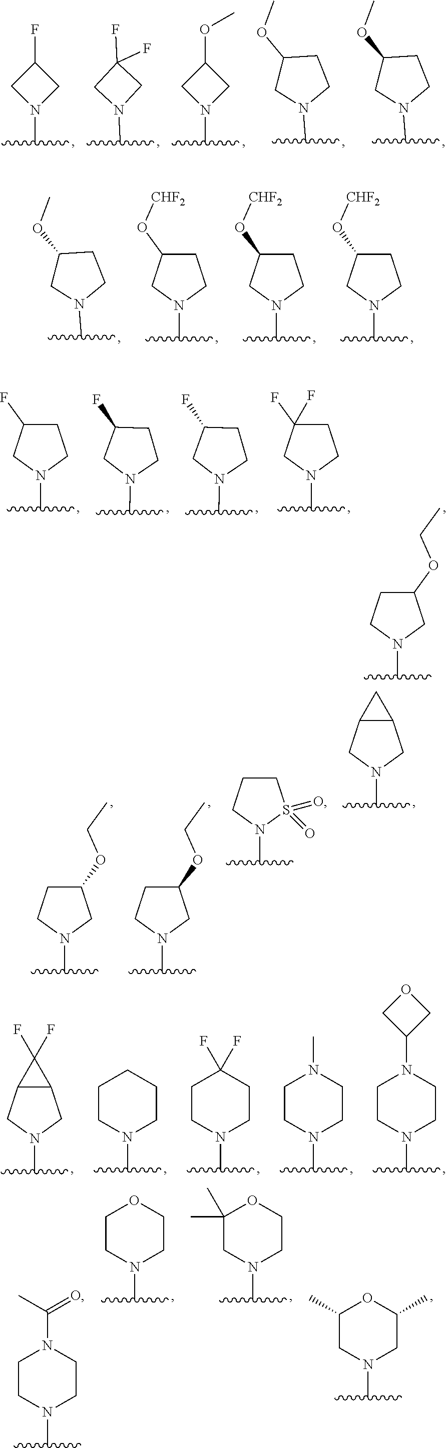

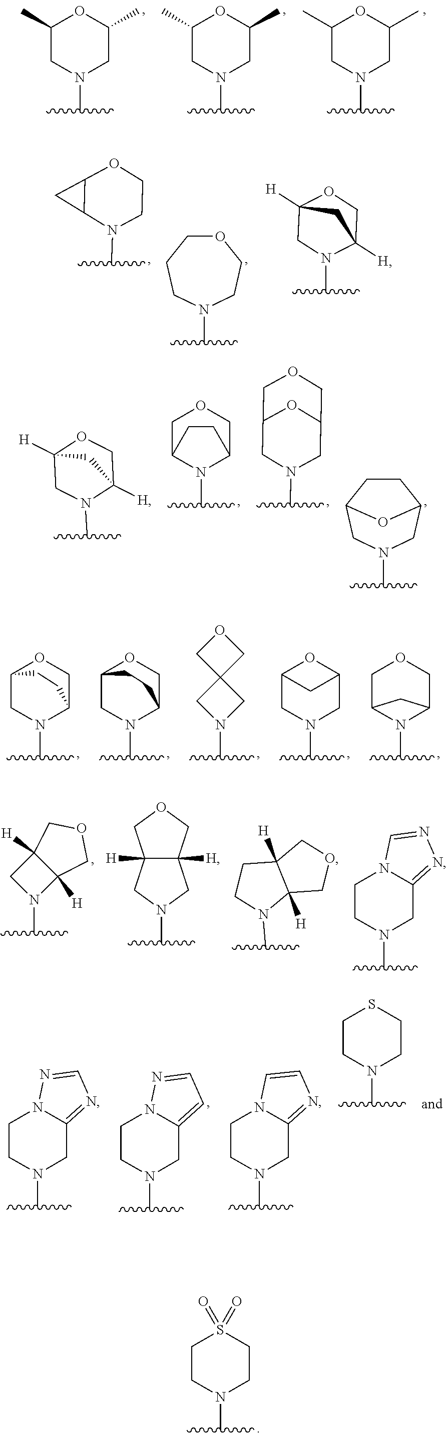

Embodiment 238. The compound of any one of embodiments 1 to 160, wherein R^{e1} is selected from the group consisting of:

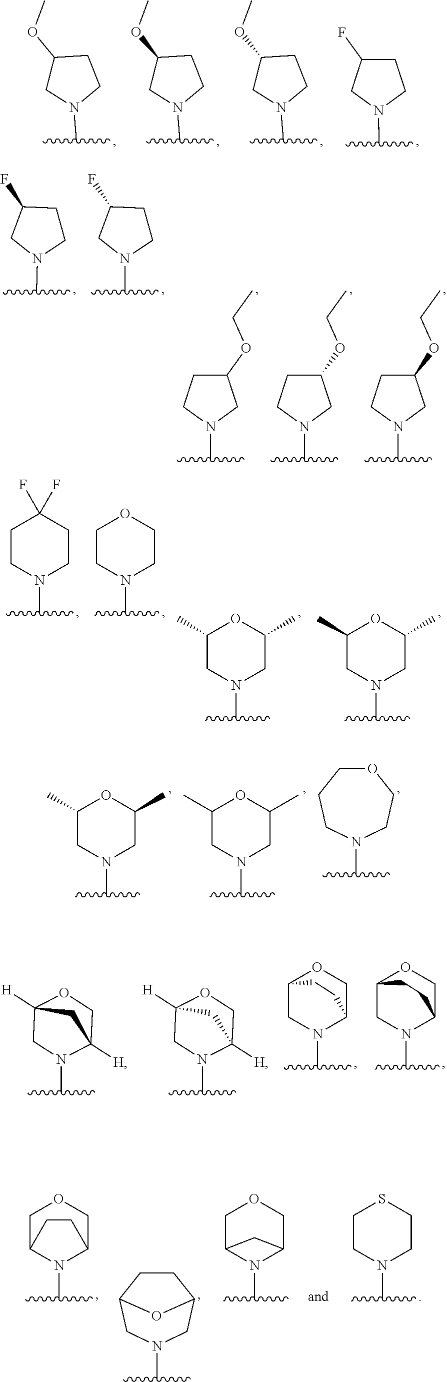

Embodiment 239. The compound of any one of embodiments 1 to 160, wherein R^{e1} is selected from the group consisting of:

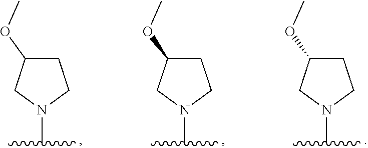

Embodiment 240. The compound of any one of embodiments 1 to 160, wherein R^{e1} is selected from the group consisting of:

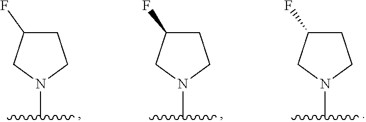 [SMILES: *N1CCC(F)C1 ; *N1CC[C@H](F)C1 ; *N1CC[C@@H](F)C1]

Embodiment 241. The compound of any one of embodiments 1 to 160, wherein R^{e1} is selected from the group consisting of:

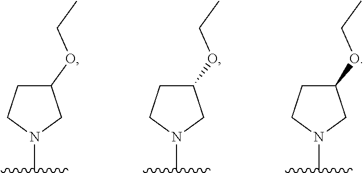

Embodiment 242. The compound of any one of embodiments 1 to 160, wherein R^{e1} is

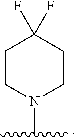 [SMILES: *N1CCC(F)(F)CC1]

Embodiment 243. The compound of any one of embodiments 1 to 160, wherein R^{e1} is selected from the group consisting of:

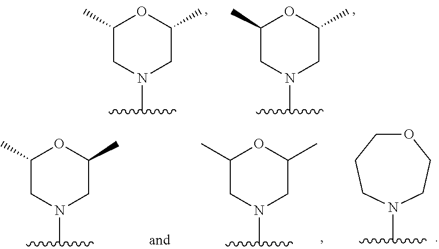

Embodiment 244. The compound of any one of embodiments 1 to 160, wherein R^{e1} is unsubstituted

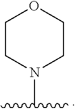 [SMILES: *N1CCOCC1]

Embodiment 245. The compound of any one of embodiments 1 to 160, wherein R^{e1} is unsubstituted

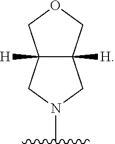

Embodiment 246. The compound of any one of embodiments 1 to 160, wherein R^{e1} is unsubstituted

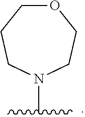 [SMILES: *N1CCCOCC1]

Embodiment 247. The compound of any one of embodiments 1 to 160, wherein R^{e1} is unsubstituted

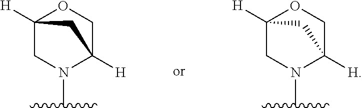

Embodiment 248. The compound of any one of embodiments 1 to 160, wherein R^{e1} is unsubstituted

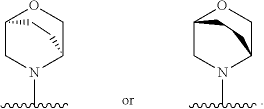 [SMILES: *N1C[C@@H]2CC[C@H]1CO2 ; *N1C[C@H]2CC[C@@H]1CO2]

Embodiment 249. The compound of any one of embodiments 1 to 160, wherein R^{e1} is unsubstituted

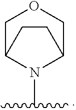 [SMILES: *N1C2CCC1COC2]

Embodiment 250. The compound of any one of embodiments 1 to 160, wherein R^{e1} is unsubstituted

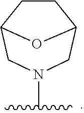 [SMILES: *N1CC2CCC(C1)O2]

Embodiment 251. The compound of any one of embodiments 1 to 160, wherein R^{e1} is unsubstituted

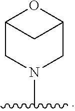 [SMILES: *N1CC2CC(C1)O2]

Embodiment 252. The compound of any one of embodiments 1 to 160, wherein R^{e1} is unsubstituted

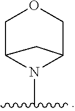 [SMILES: *N1C2COCC1C2]

Embodiment 253. The compound of any one of embodiments 1 to 160, wherein R^{e1} is unsubstituted

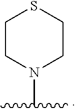 [SMILES: *N1CCSCC1]

Embodiment 254. The compound of any one of embodiments 187 to 253, wherein R^{2c} is

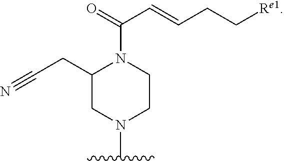

Embodiment 255. The compound of any one of embodiments 187 to 253, wherein R^{2c} is

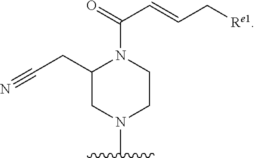

Embodiment 256. The compound of any one of embodiments 1 to 159, wherein R^{e} is R^{e2}.
Embodiment 257. The compound of any one of embodiments 1 to 159, 161 to 185, 187 to 253 and 256, wherein R^{e2} is a 5-6 membered heteroaryl group containing at least one nitrogen atom, wherein the attachment point for the heteroaryl group is a carbon atom group and wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 258. The compound of any one of embodiments 1 to 159, 161 to 185, 187 to 253 and 256, wherein R^{e2} is selected from the group consisting of pyrimidinyl, pyrazinyl, oxazolyl, 1,2,4-oxadiazolyl, 1,3,4-oxadiazolyl, 1H-1,2,4-triazolyl, imidazolyl, 4H-1,2,4-triazolyl, 1H-1,2,4-triazolyl, 2-H-tetrazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 259. The compound of any one of embodiments 1 to 159, 161 to 185, 187 to 253 and 256, wherein R^{e2} is selected from the group consisting of pyrimidinyl, pyrazinyl, oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 260. The compound of any one of embodiments 1 to 159, 161 to 185, 187 to 253 and 256, wherein R^{e2} is selected from the group consisting of pyrimidinyl, oxazolyl, 1,2,4-oxadiazolyl and 1,2,4-thiadiazolyl each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 261. The compound of any one of embodiments 1 to 159, 161 to 185, 187 to 253 and 256, wherein R^{e2} is selected from the group consisting of pyrimidinyl and 1,2,4-oxadiazolyl each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 262. The compound of any one of embodiments 1 to 159, 161 to 185, 187 to 253 and 256, wherein R^{e2} is selected from the group consisting of

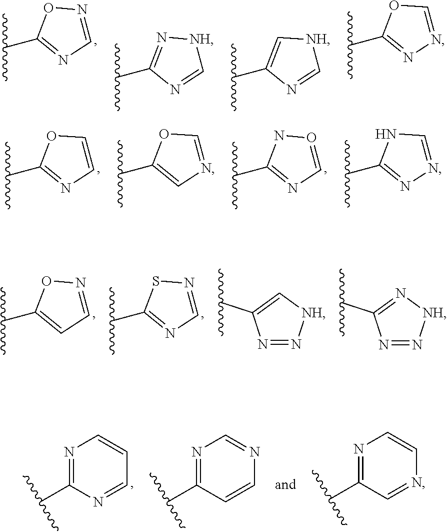

each substituted with 0, 1 or 2 substituents independently selected from halo, hydroxy, C₁-C₄ acyclic alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 263. The compound of any one of embodiments 1 to 159, 161 to 185, 187 to 253 and 256 wherein R^{e2} is selected from the group consisting of

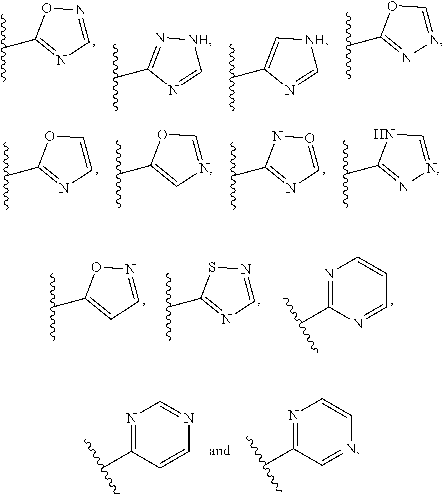

each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 264. The compound of any one of embodiments 1 to 159, 161 to 185, 187 to 253 and 256, wherein R^{e2} is selected from the group consisting of

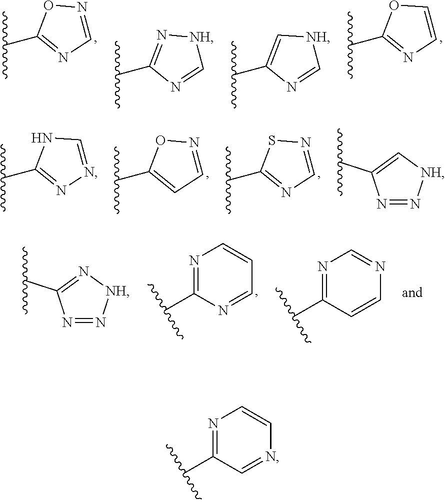

each substituted with 0, 1 or 2 substituents independently selected from halo, hydroxy, C₁-C₄ acyclic alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 265. The compound of any one of embodiments 1 to 159, 161 to 185, 187 to 253 and 256, wherein R^{e2} is selected from the group consisting of

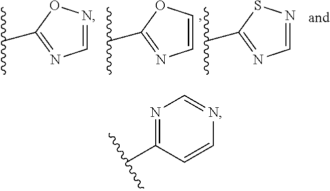

each substituted with 0, 1 or 2 substituents independently selected from halo, C₁-C₄ acyclic alkyl, C₁-C₄ hydroxyalkyl, C₁-C₄ haloalkyl, C₃-C₆ heterocyclyl and C₃-C₆ cycloalkyl optionally substituted with one or two instances of halo or methyl.
Embodiment 266. The compound of any one of embodiments 1 to 159, 161 to 185, 187 to 253 and 256, wherein R^{e2} is selected from the group consisting of

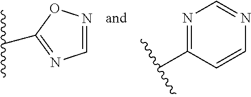 [SMILES: *c1ncno1 ; *c1ccncn1]

each substituted with 0, 1 or 2 substituents independently selected from halo, C₁-C₄ acyclic alkyl, C₁-C₄ hydroxyalkyl, C₁-C₄ haloalkyl, C₃-C₆ heterocyclyl and C₃-C₆ cycloalkyl optionally substituted with one or two instances of halo or methyl.
Embodiment 267. The compound of any one of embodiments 1 to 159, 161 to 185, 187 to 253 and 256, wherein R^{e2} is selected from the group consisting of

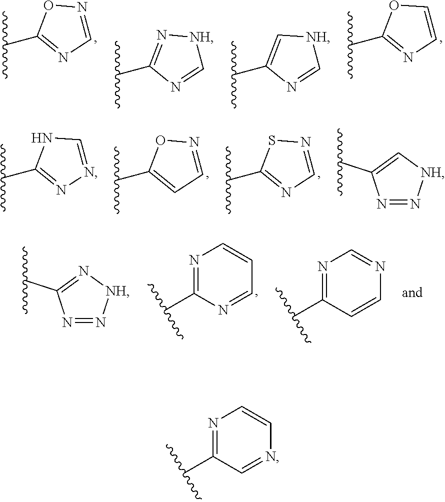

each substituted with 0, 1 or 2 substituents independently selected from —F, -Me, -Et, -ⁱPr, -tBu, —C(OH)(CH₃)₂, —CHF₂, —CF₂CH₃, —CH(F)CH₃, —CF₃, oxetanyl, cyclopropyl, 1-Me-cyclopropyl and 2-F-cyclopropyl.
Embodiment 268. The compound of any one of embodiments 1 to 159, 161 to 185, 187 to 253 and 256, wherein R^{e2} is selected from the group consisting of

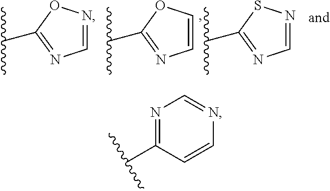

each substituted with 0, 1 or 2 substituents independently selected from —F, -Me, -Et, -ⁱPr, -tBu, —C(OH)(CH₃)₂, —CHF₂, —CF₂CH₃, —CH(F)CH₃, —CF₃, oxetanyl, cyclopropyl, 1-Me-cyclopropyl and 2-F-cyclopropyl.
Embodiment 269. The compound of any one of embodiments 1 to 159, 161 to 185, 187 to 253 and 256, wherein R^{e2} is selected from the group consisting of

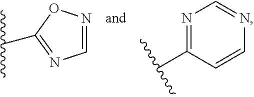 [SMILES: *c1ncno1 ; *c1ccncn1]

each substituted with 0, 1 or 2 substituents independently selected from —F, -Me, -Et, -ⁱPr, -tBu, —C(OH)(CH₃)₂, —CHF₂, —CF₂CH₃, —CH(F)CH₃, —CF₃, oxetanyl, cyclopropyl, 1-Me-cyclopropyl and 2-F-cyclopropyl.
Embodiment 270. The compound of any one of embodiments 1 to 159, 161 to 185, 187 to 253 and 256, wherein R^{e2} is selected from the group consisting of:

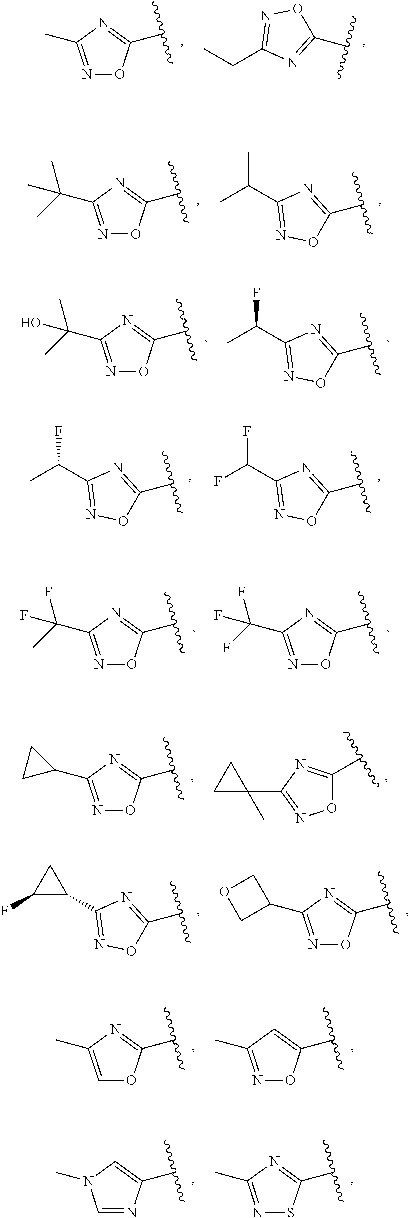

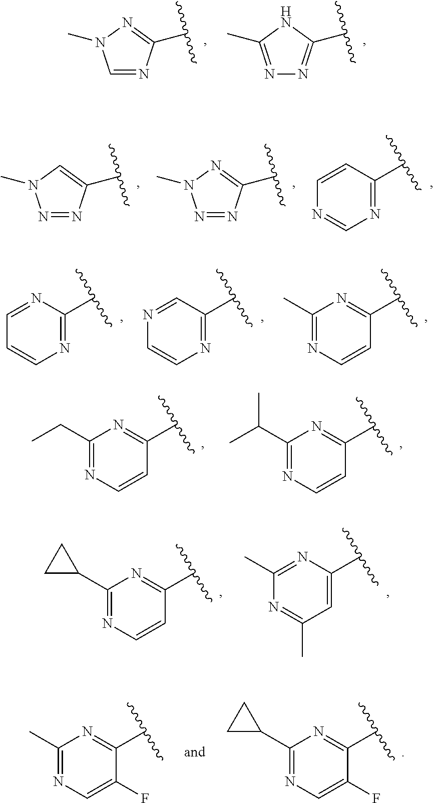

Embodiment 271. The compound of any one of embodiments 1 to 159, 161 to 185, 187 to 253 and 256, wherein R^{e2} is selected from the group consisting of:

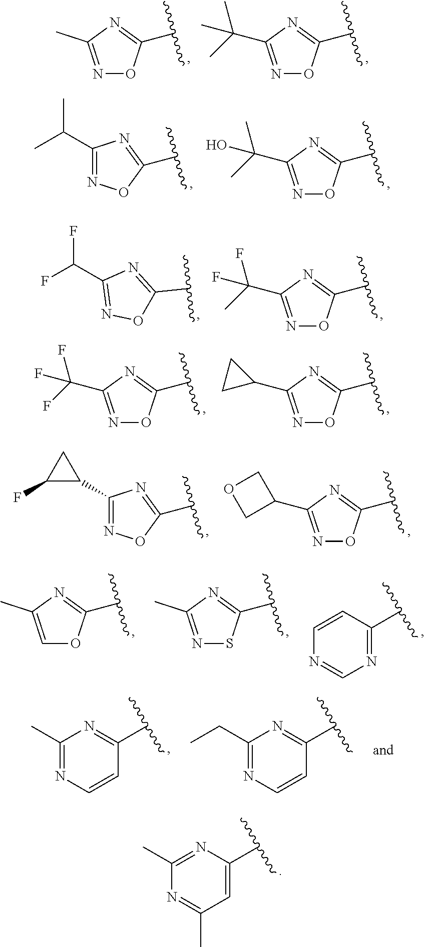

Embodiment 272. The compound of any one of embodiments 1 to 159, 161 to 185, 187 to 253 and 256, wherein R^{e2} is selected from the group consisting of:

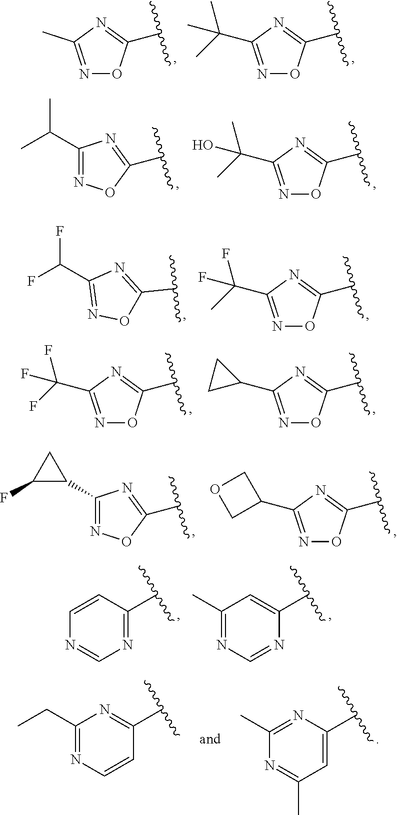

Embodiment 273. The compound of any one of embodiments 1 to 159, 161 to 185, 187 to 253 and 256, wherein R^{e2} is a 6 membered heteroaryl group substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 274. The compound of any one of embodiments 1 to 159, 161 to 185, 187 to 253 and 256, wherein R^{e2} is a 6 membered heteroaryl group substituted with 0, 1 or 2 substituents independently selected from halo, acyclic C₁-C₄ alkyl, and C₃-C₆ cycloalkyl.
Embodiment 275. The compound of any one of embodiments 1 to 159, 161 to 185, 187 to 253 and 256, wherein R^{e2} is a 6 membered heteroaryl group substituted with 0, 1 or 2 substituents independently selected from —F, -Me, -Et, -ⁱPr, and cyclopropyl.
Embodiment 276. The compound of any one of embodiments 1 to 159, 161 to 185, 187 to 253 and 256, wherein R^{e2} is pyrimidinyl or pyridazinyl substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 277. The compound of any one of embodiments 1 to 159, 161 to 185, 187 to 253 and 256, wherein R^{e2} is unsubstituted pyrimidinyl or pyridazinyl.
Embodiment 278. The compound of any one of embodiments 1 to 159, 161 to 185, 187 to 253 and 256, wherein R^{e2} is pyrimidinyl substituted with 0, 1 or 2 substituents independently selected from halo, acyclic C₁-C₄ alkyl, and C₃-C₆ cycloalkyl.
Embodiment 279. The compound of any one of embodiments 1 to 159, 161 to 185, 187 to 253 and 256, wherein R^{e2} is pyrimidinyl substituted with 0, 1 or 2 substituents independently selected from —F, -Me, -Et, -ⁱPr, and cyclopropyl.
Embodiment 280. The compound of any one of embodiments 1 to 159, 161 to 185, 187 to 253 and 256, wherein R^{e2} is selected from the group consisting of

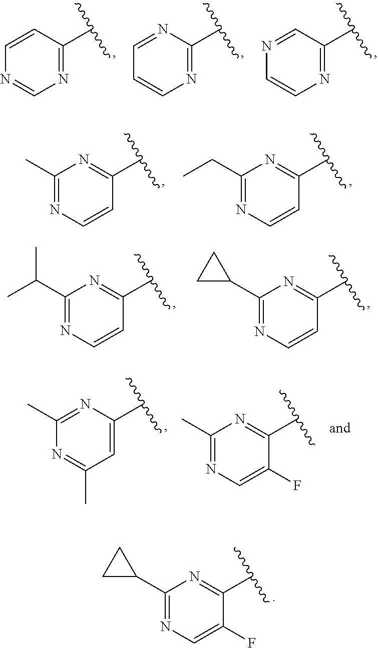

Embodiment 281. The compound of any one of embodiments 1 to 159, 161 to 185, 187 to 253 and 256, wherein R^{e2} is selected from the group consisting of

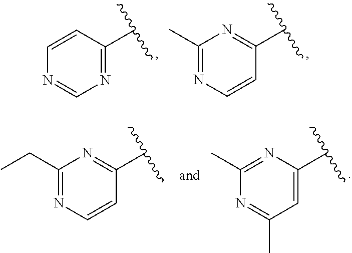

Embodiment 282. The compound of any one of embodiments 1 to 159, 161 to 185, 187 to 253 and 256, wherein R^{e2} is selected from the group consisting of

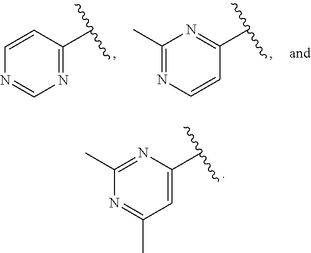

Embodiment 283. The compound of any one of embodiments 1 to 159, 161 to 185, 187 to 253 and 256, wherein R^{e2} is

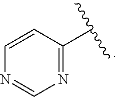 [SMILES: *c1ccncn1]

Embodiment 284. The compound of any one of embodiments 1 to 159, 161 to 185, 187 to 253 and 256, wherein R^{e2} is

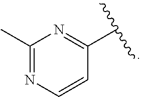 [SMILES: *c1ccnc(C)n1]

Embodiment 285. The compound of any one of embodiments 1 to 159, 161 to 185, 187 to 253 and 256, wherein R^{e2} is

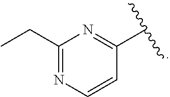 [SMILES: *c1ccnc(CC)n1]

Embodiment 286. The compound of any one of embodiments 1 to 159, 161 to 185, 187 to 253 and 256, wherein R^{e2} is

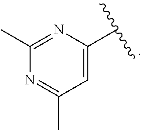 [SMILES: *c1cc(C)nc(C)n1]

Embodiment 287. The compound of any one of embodiments 1 to 159, 161 to 185, 187 to 253 and 256, wherein R^{e2} is selected from the group consisting of

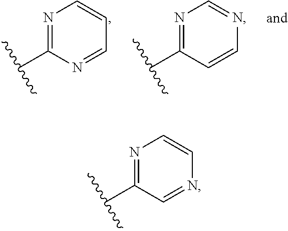 [SMILES: *c1ncccn1 ; *c1ccncn1 ; "and" ; *c1cnccn1]

Embodiment 288. The compound of any one of embodiments 1 to 159, 161 to 185, 187 to 253 and 256, wherein R^{e2} is a 5 membered heteroaryl group containing at least one nitrogen atom, wherein the attachment point for the heteroaryl group is a carbon atom group and wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 289. The compound of embodiment 288, wherein R^{e2} is selected from the group consisting of oxazolyl, 1,2,4-oxadiazolyl, 1,3,4-oxadiazolyl, 1H-1,2,4-triazolyl, imidazolyl, 4H-1,2,4-triazolyl, 1H-1,2,4-triazolyl, 2-H-tetrazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 290. The compound of embodiment 288, wherein R^{e2} is selected from the group consisting of oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 291. The compound of embodiment 288, wherein R^{e2} is selected from the group consisting of oxazolyl, 1,2,4-oxadiazolyl and 1,2,4-thiadiazolyl each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 292. The compound of embodiment 288, wherein R^{e2} is selected from the group consisting of

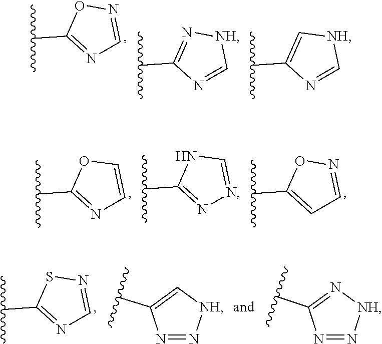

each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 293. The compound of embodiment 288, wherein R^{e2} is selected from the group consisting of

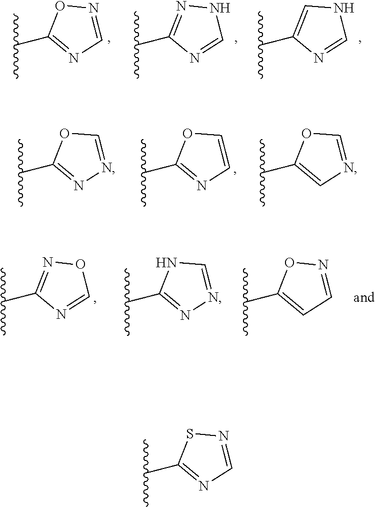

each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 294. The compound of embodiment 288, wherein R^{e2} is selected from the group consisting of

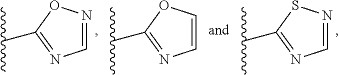

each substituted with 0 or 1 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 295. The compound of embodiment 288, wherein R^{e2} is

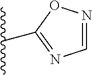 [SMILES: *c1ncno1]

substituted with 0 or 1 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 296. The compound of any one of embodiments 288 to 295, wherein the 5 membered heteroaryl group of R^{e2} is substituted with 0 or 1 substituents independently selected from acyclic C₁-C₄ alkyl, C₁-C₄ hydroxyalkyl, C₁-C₄ haloalkyl, C₃-C₆ heterocyclyl and C₃-C₆ cycloalkyl optionally substituted with one or two instances of halo or methyl.
Embodiment 297. The compound of any one of embodiments 288 to 295, wherein the 5-membered heteroaryl group of R^{e2} is substituted with 0 or 1 substituents independently selected from C₁-C₄ alkyl, C₁-C₄ hydroxyalkyl, C₁-C₄ haloalkyl, C₃-C₆ heterocyclyl and C₃-C₆ cycloalkyl optionally substituted with one or two instances of halo or methyl.
Embodiment 298. The compound of any one of embodiments 288 to 295, wherein the 5-membered heteroaryl group of R^{e2} is substituted with 0 or 1 substituents independently selected from methyl, ethyl, isopropyl, tert-butyl, difluoromethyl, 1,1-difluoroethyl, —C(OH)(CH₃)₂, oxetan-3-yl, cyclopropyl, 1-methylcyclopropyl and 2-fluorocyclopropyl.
Embodiment 299. The compound of any one of embodiments 288 to 295, wherein the 5 membered heteroaryl group of R^{e2} is substituted with 0 or 1 substituents independently selected from -Me, -Et, -ⁱPr, -tBu, —C(OH)(CH₃)₂, —CHF₂, —CF₂CH₃, —CH(F)CH₃, —CF₃, oxetanyl, cyclopropyl, 1-Me-cyclopropyl and 2-F-cyclopropyl.
Embodiment 300. The compound of embodiment 288, wherein R^{e2} is selected from the group consisting of:

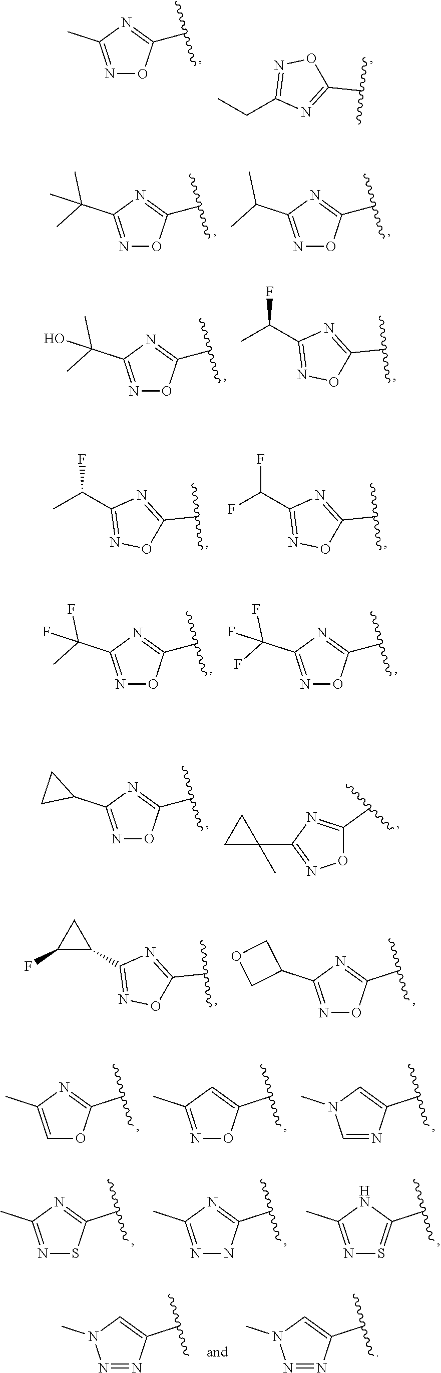

Embodiment 301. The compound of embodiment 288, wherein R^{e2} is selected from the group consisting of:

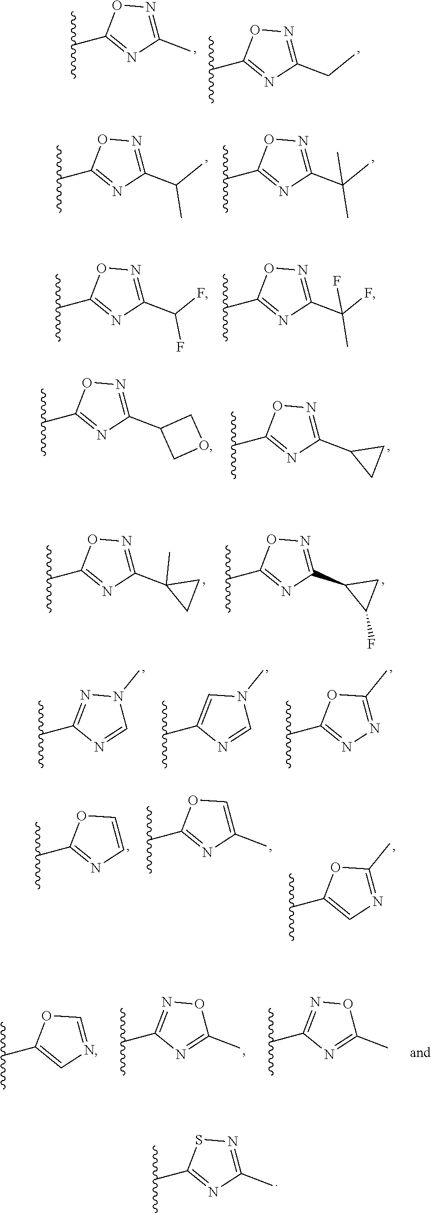

Embodiment 302. The compound of embodiment 288, wherein R^{e2} is selected from the group consisting of:

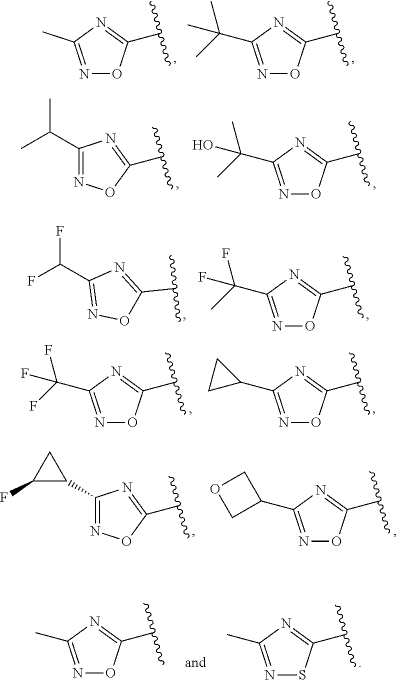

Embodiment 303. The compound of embodiment 288, wherein R^{e2} is 1,2,4-oxadiazolyl substituted with 1 substituent selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 304. The compound of embodiment 288, wherein R^{e2} is

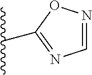 [SMILES: *c1ncno1]

substituted with 1 substituent selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.
Embodiment 305. The compound of embodiment 288, wherein the oxadiazolyl is substituted with one substituent selected from methyl, ethyl, isopropyl, tert-butyl, difluoromethyl, 1,1-difluoroethyl, —C(OH)(CH₃)₂, oxetan-3-yl, cyclopropyl, 1-methylcyclopropyl and 2-fluorocyclopropyl.
Embodiment 306. The compound of embodiment 288, wherein R^{e2} is selected from the group consisting of:

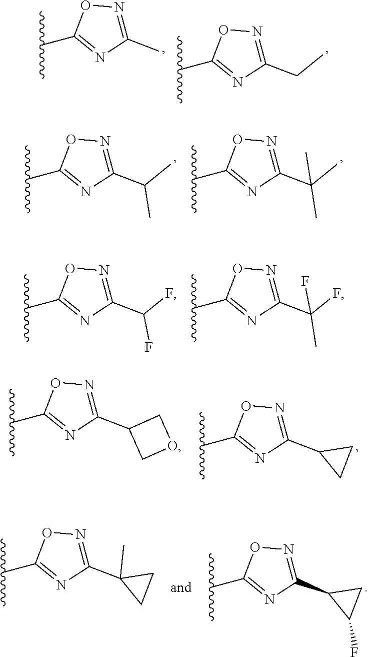

Embodiment 307. The compound of embodiment 288, wherein R^{e2} is selected from the group consisting of:

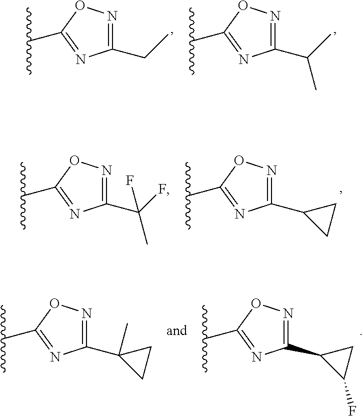

Embodiment 308. The compound of any one of embodiments 256 to 307, wherein R^{2c} is

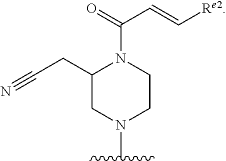

Embodiment 309. The compound of any one of embodiments 1 to 159, wherein R^{e} is R^{e3}.
Embodiment 310. The compound of any one of embodiments 1 to 159, 161 to 185, 187 to 253 and 257 to 309, wherein R^{e3} is —NR⁵R⁶, wherein R⁵ is -Me and R⁶ is independently selected from C₁-C₄ alkyl, C₁-C₆ alkoxy and —CH₂-(4-6 membered heterocycle).
Embodiment 311. The compound of any one of embodiments 1 to 159, 161 to 185, 187 to 253 and 257 to 309, wherein R^{e3} is —NR⁵R⁶, wherein R⁵ is -Me and R⁶ is independently selected from -Me, -Et, —Pr, -ⁱPr, —CH₂CH₂OMe and

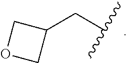 [SMILES: *CC1COC1]

Embodiment 312. The compound of any one of embodiments 1 to 159, 161 to 185, 187 to 253 and 257 to 309, wherein R^{e3} is selected from

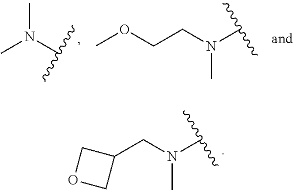

Embodiment 313. The compound of any one of embodiments 1 to 159, 161 to 185, 187 to 253 and 257 to 309, wherein R^{e3} is

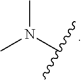 [SMILES: *N(C)C]

Embodiment 314. The compound of any one of embodiments 309 to 313 wherein R^{2c} is

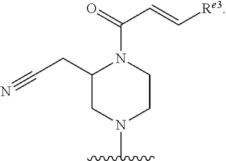

Embodiment 315. The compound of any one of embodiments 1 to 314, wherein the stereochemistry of the methylene nitrile group of R^{2c} is (S).
Embodiment 316. The compound of any one of claims 1 to 315, wherein R¹ is

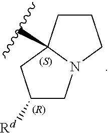

Embodiment 317. The compound of any one of embodiments 1 to 316, wherein the compound is selected from the group consisting of:

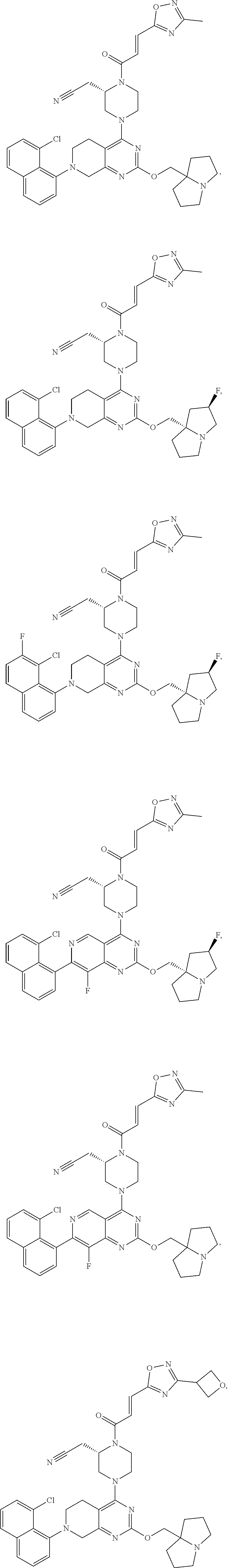

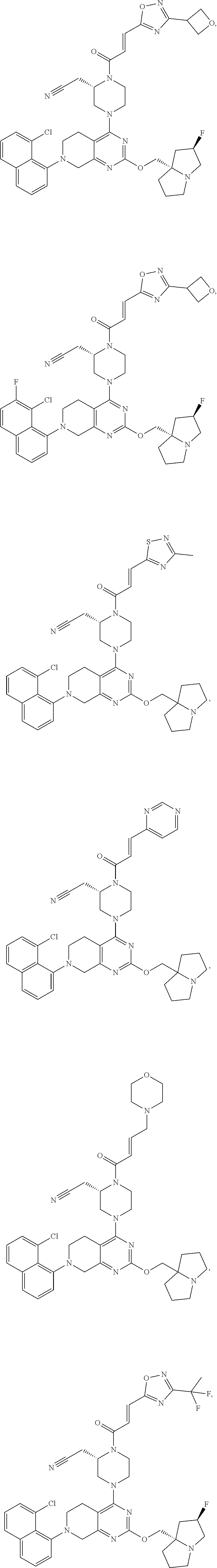

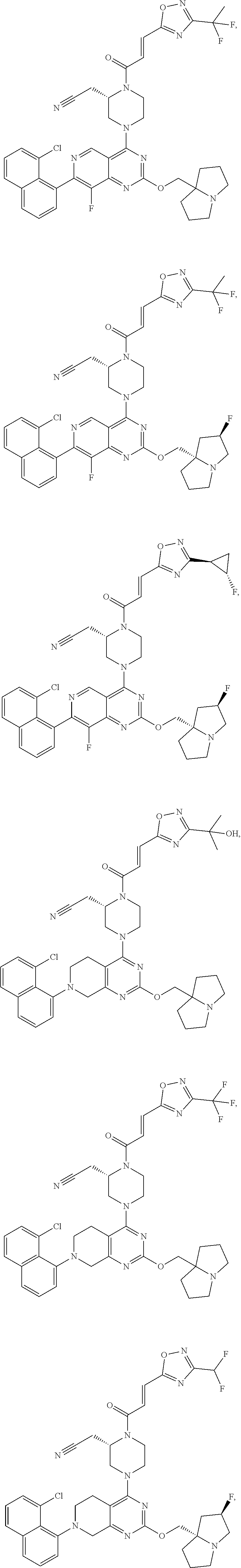

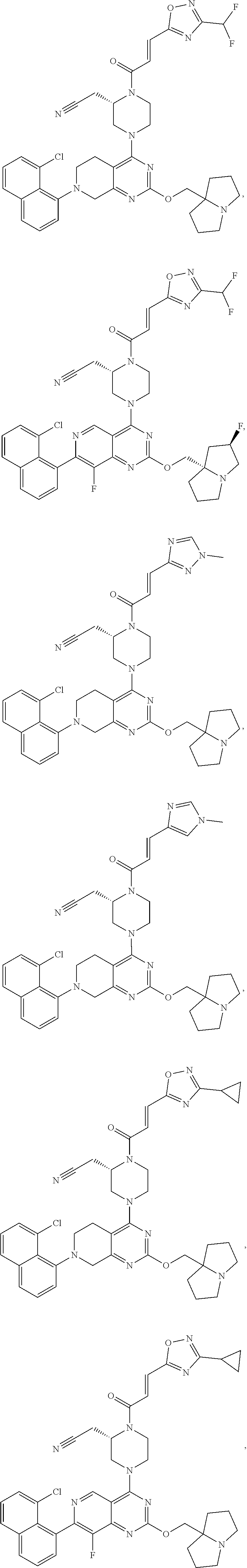

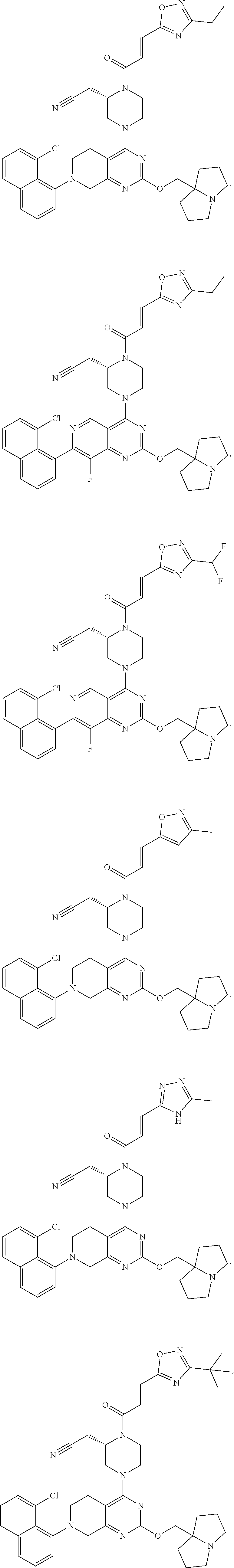

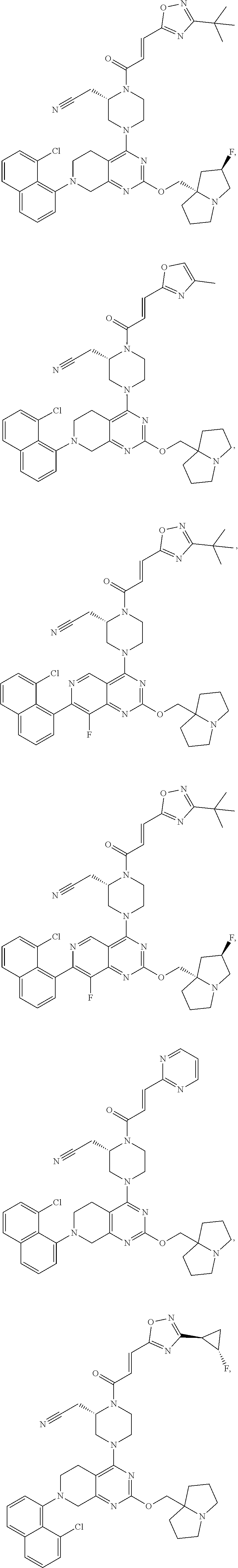

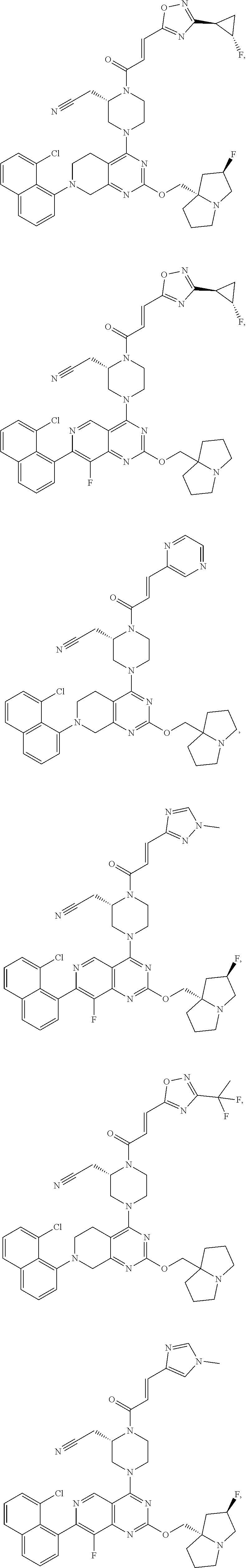

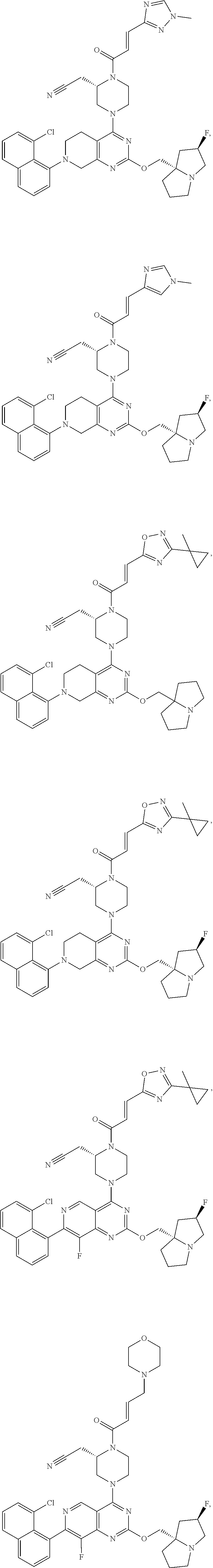

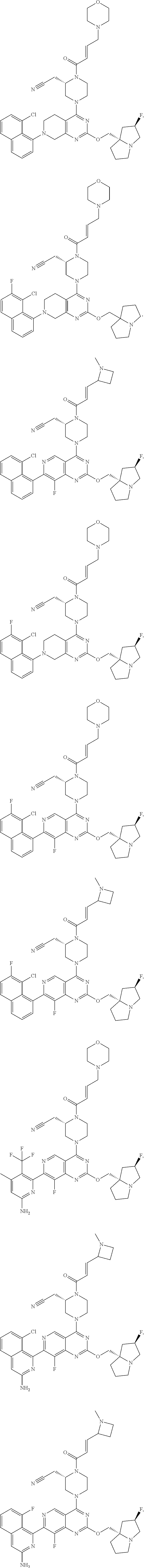

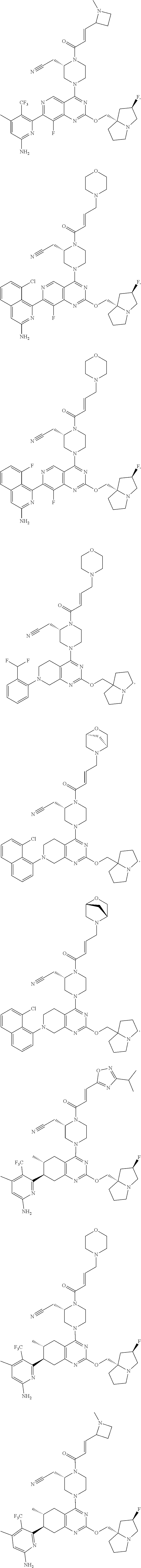

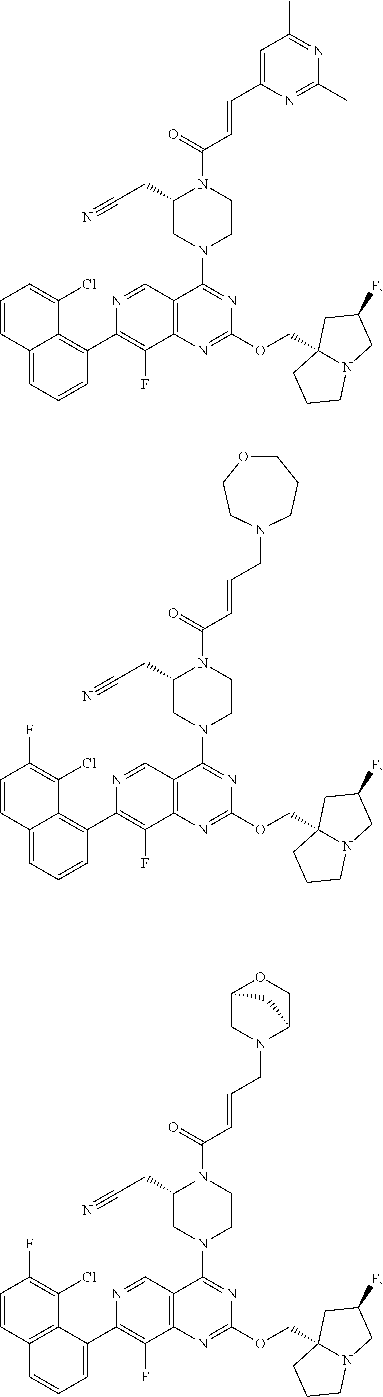 [SMILES: Cc1cc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)nc(C)n1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCCOCC1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@@H]2C[C@H]1CO2]

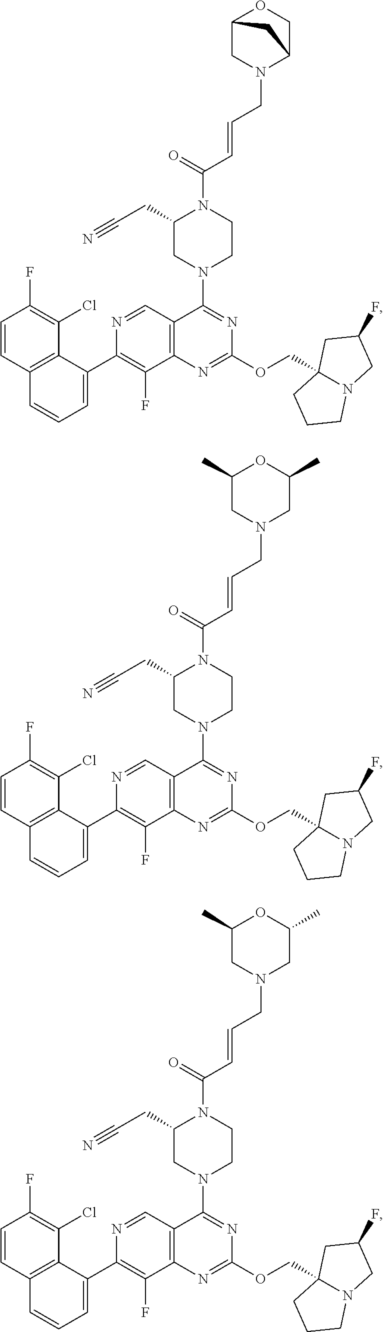

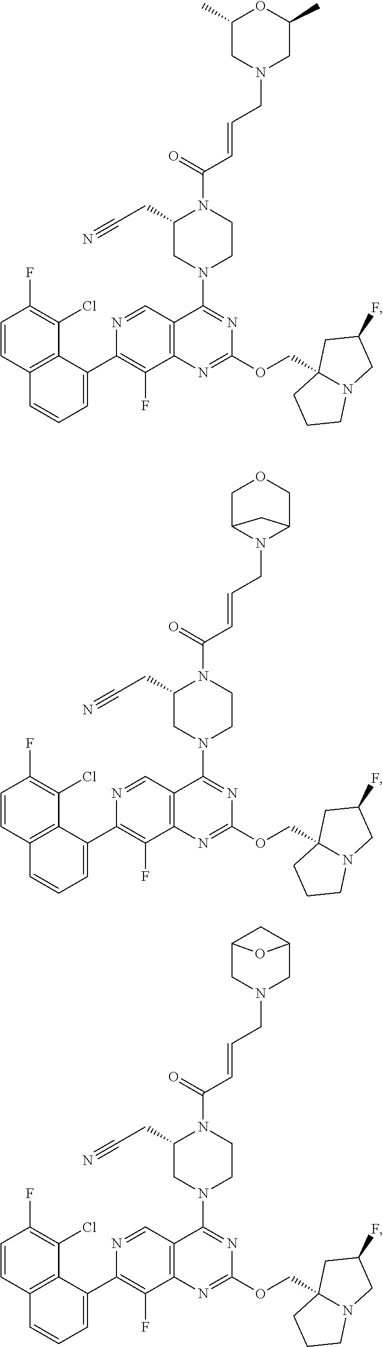 [SMILES: C[C@H]1CN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C[C@H](C)O1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C2COCC1C2 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2CC(C1)O2]

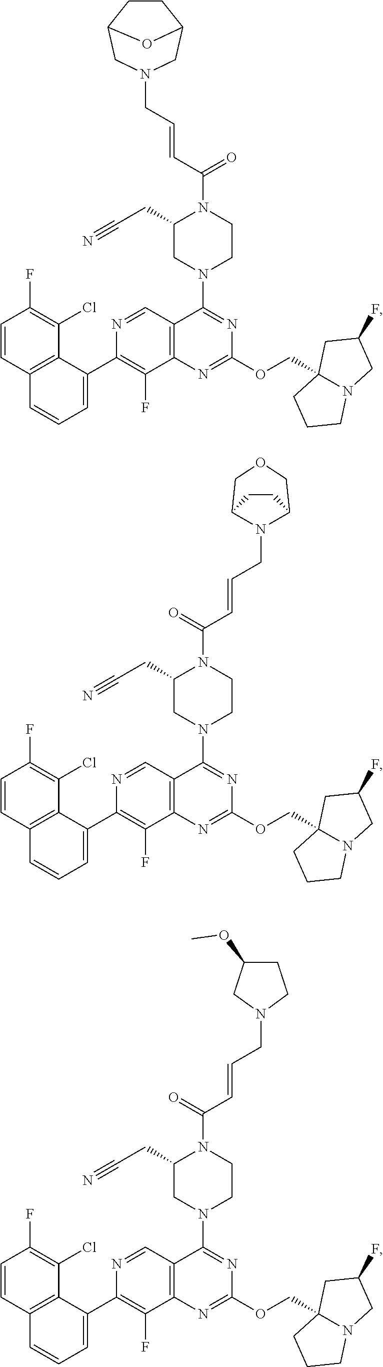 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2CCC(C1)O2 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1[C@@H]2CC[C@H]1COC2 ; CO[C@H]1CCN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C1]

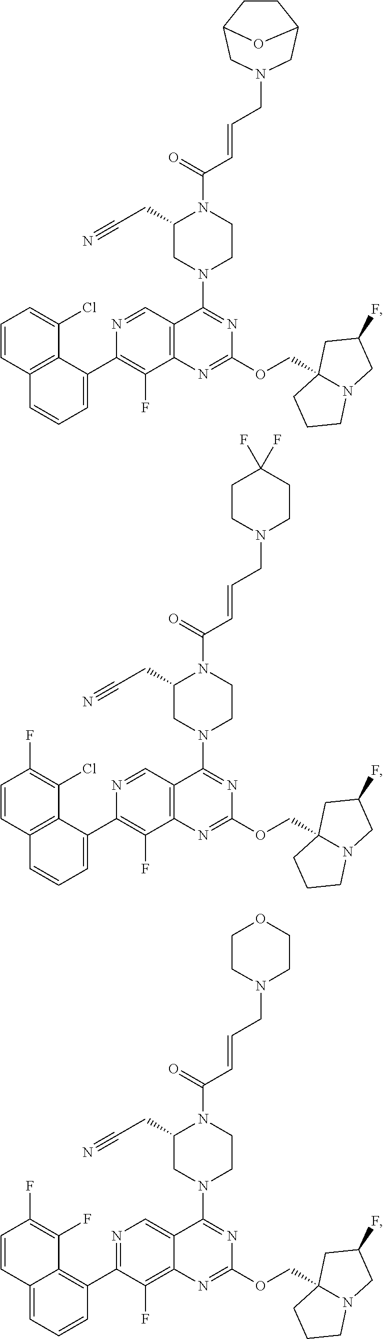

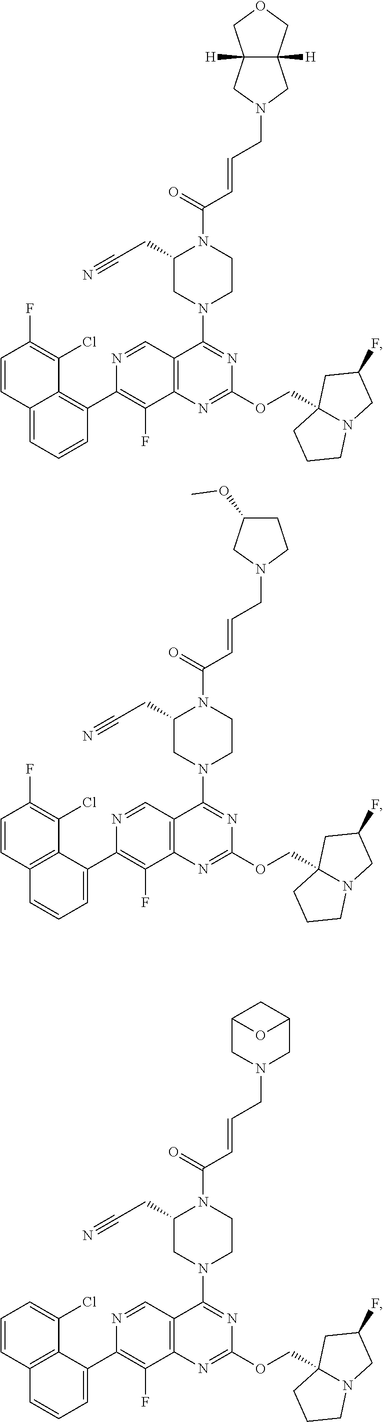 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@H]2COC[C@H]2C1 ; CO[C@@H]1CCN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CC(C1)O2]

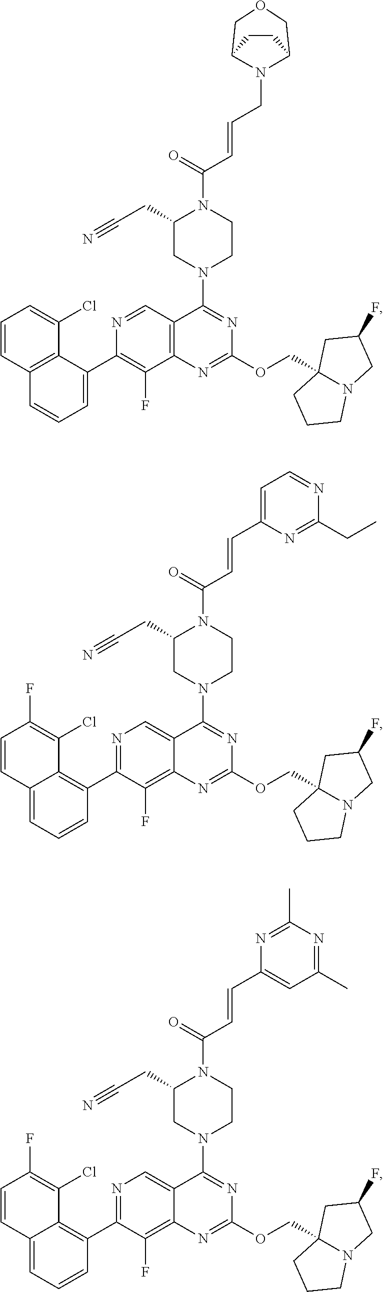 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1[C@@H]2CC[C@H]1COC2 ; CCc1nccc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1 ; Cc1cc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)nc(C)n1]

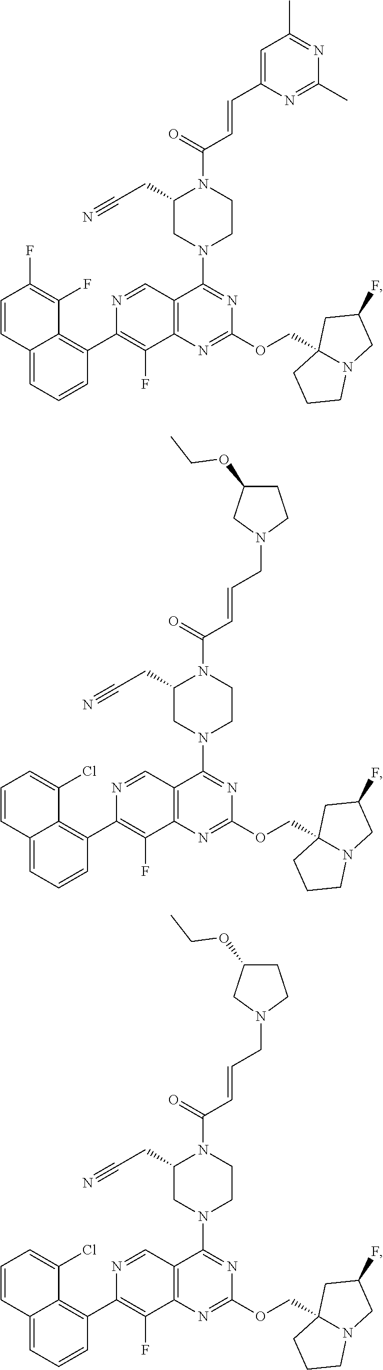 [SMILES: Cc1cc(/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)nc(C)n1 ; CCO[C@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1 ; CCO[C@@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1]

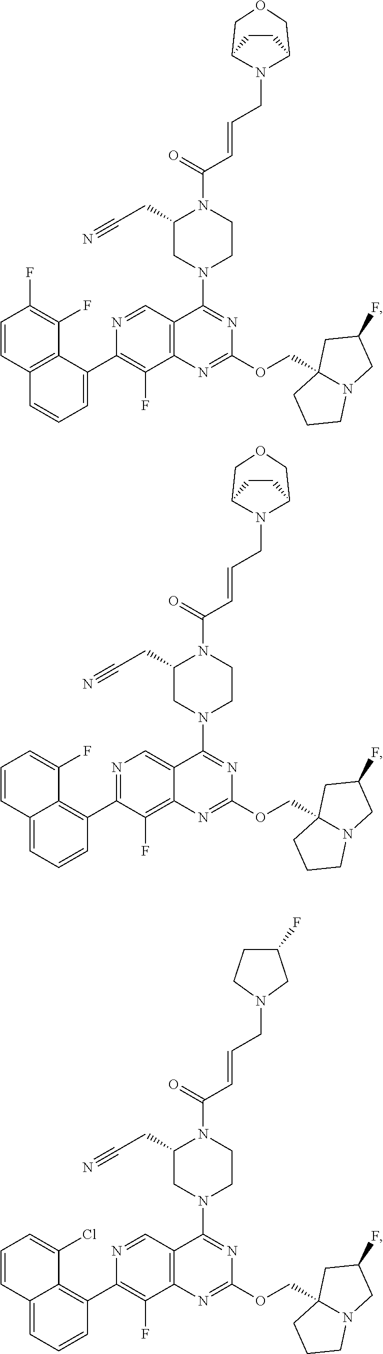 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1[C@@H]2CC[C@H]1COC2 ; N#CC[C@H]1CN(c2nc(OC[C@@]34CCCN3C[C@H](F)C4)nc3c(F)c(-c4cccc5cccc(F)c45)ncc23)CCN1C(=O)/C=C/CN1[C@@H]2CC[C@H]1COC2 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@H](F)C1]

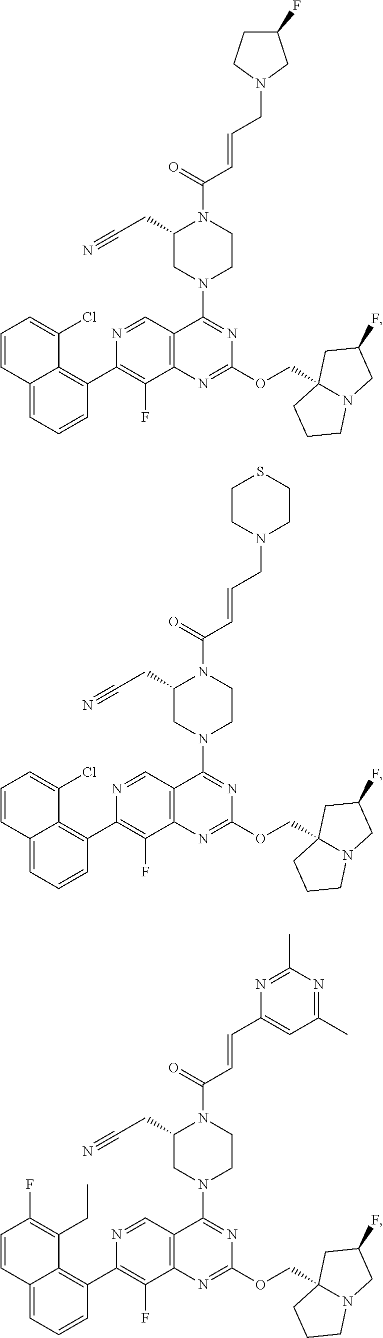

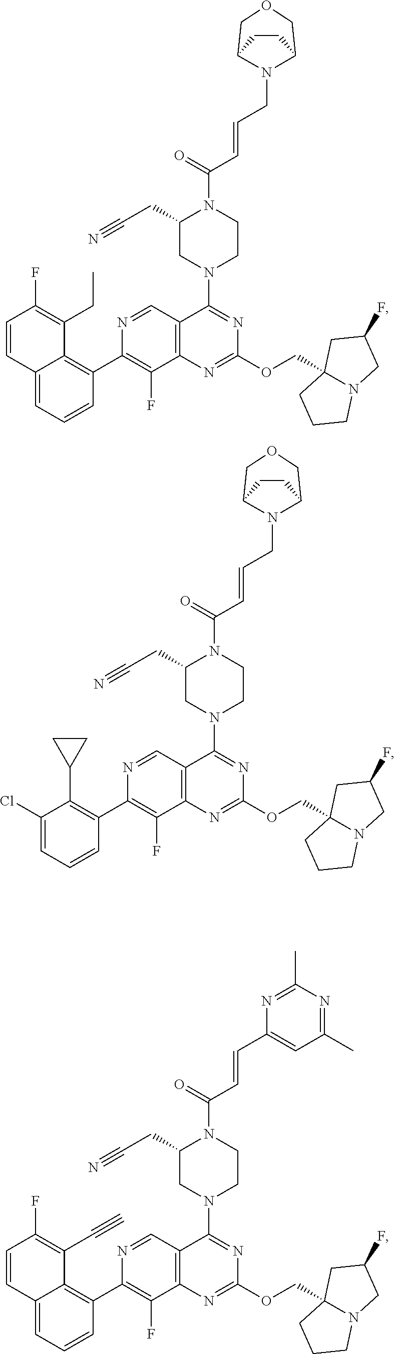

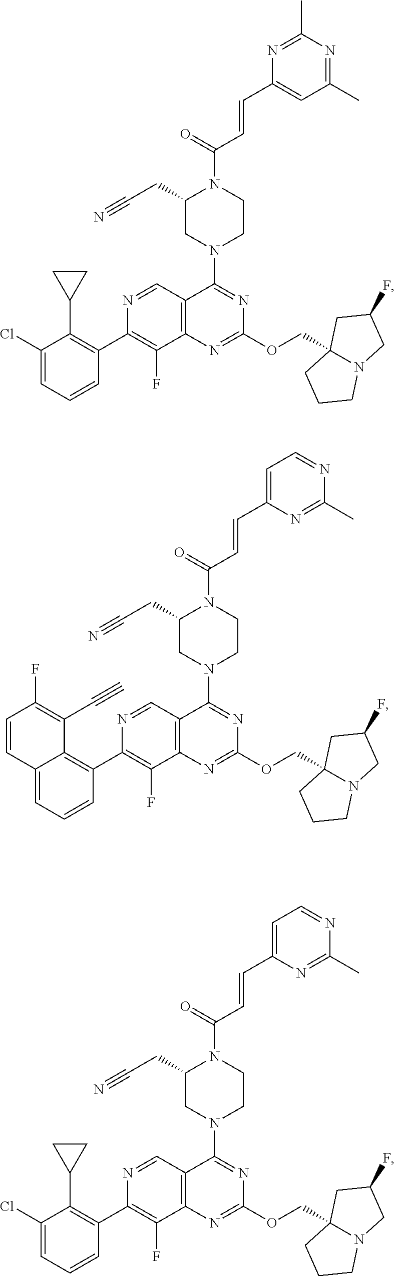 [SMILES: Cc1cc(/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)nc(C)n1 ; C#Cc1c(F)ccc2cccc(-c3ncc4c(N5CCN(C(=O)/C=C/c6ccnc(C)n6)[C@@H](CC#N)C5)nc(OC[C@@]56CCCN5C[C@H](F)C6)nc4c3F)c12 ; Cc1nccc(/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)n1]

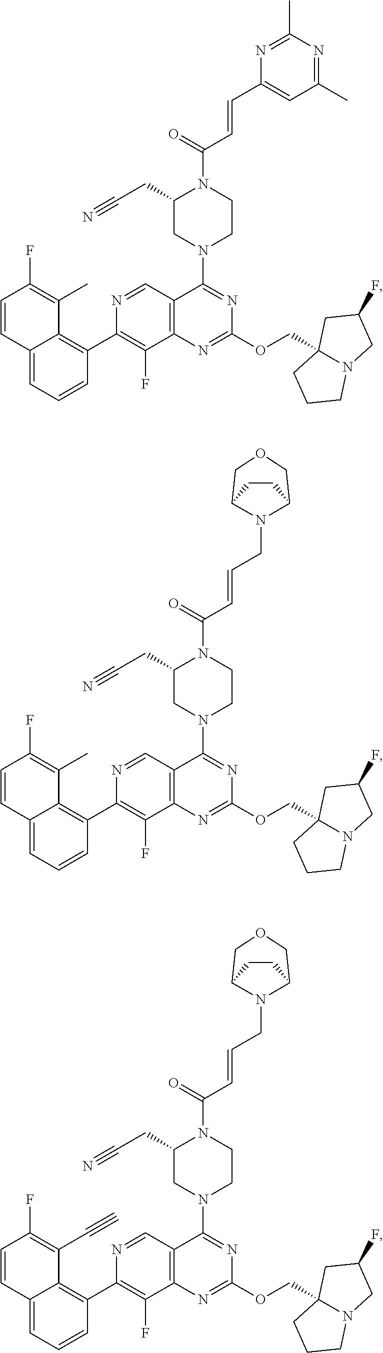 [SMILES: Cc1cc(/C=C/C(=O)N2CCN(c3nc(OC[C@@]45CCCN4C[C@H](F)C5)nc4c(F)c(-c5cccc6ccc(F)c(C)c56)ncc34)C[C@@H]2CC#N)nc(C)n1 ; Cc1c(F)ccc2cccc(-c3ncc4c(N5CCN(C(=O)/C=C/CN6[C@@H]7CC[C@H]6COC7)[C@@H](CC#N)C5)nc(OC[C@@]56CCCN5C[C@H](F)C6)nc4c3F)c12 ; C#Cc1c(F)ccc2cccc(-c3ncc4c(N5CCN(C(=O)/C=C/CN6[C@@H]7CC[C@H]6COC7)[C@@H](CC#N)C5)nc(OC[C@@]56CCCN5C[C@H](F)C6)nc4c3F)c12]

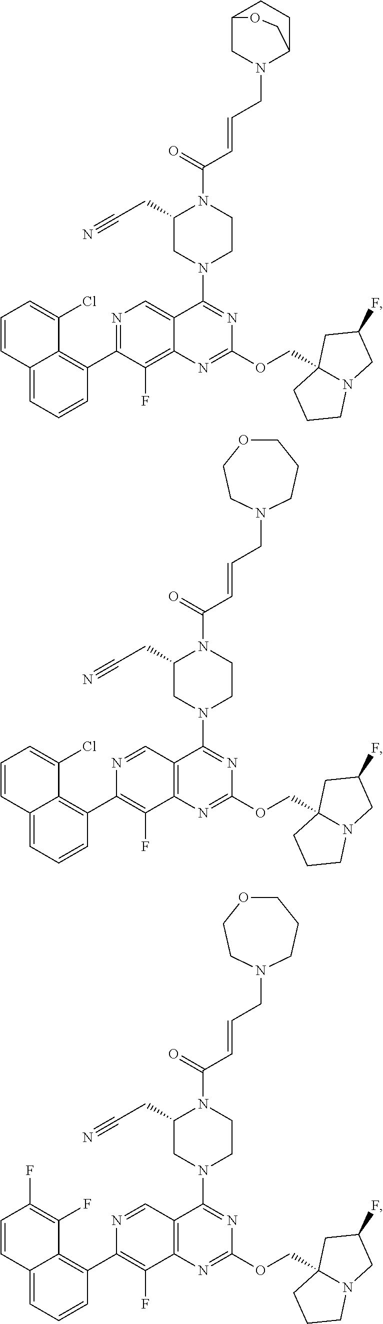

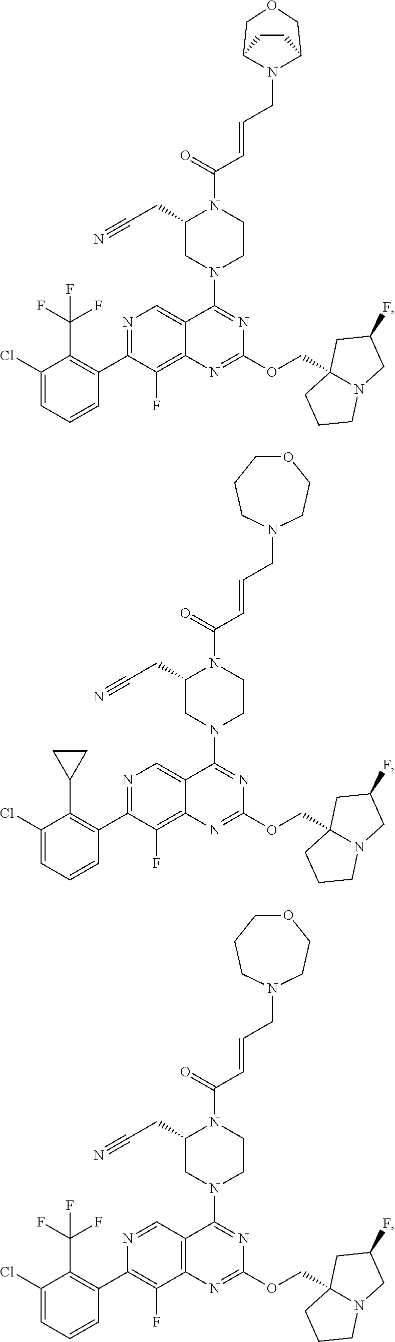

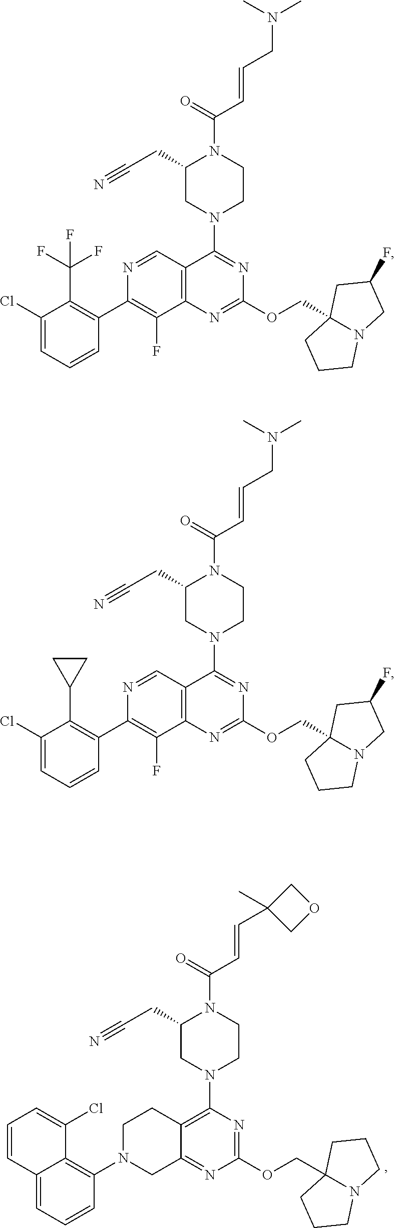 [SMILES: CN(C)C/C=C/C(=O)N1CCN(c2nc(OC[C@@]34CCCN3C[C@H](F)C4)nc3c(F)c(-c4cccc(Cl)c4C(F)(F)F)ncc23)C[C@@H]1CC#N ; CN(C)C/C=C/C(=O)N1CCN(c2nc(OC[C@@]34CCCN3C[C@H](F)C4)nc3c(F)c(-c4cccc(Cl)c4C4CC4)ncc23)C[C@@H]1CC#N ; CC1(/C=C/C(=O)N2CCN(c3nc(OCC45CCCN4CCC5)nc4c3CCN(c3cccc5cccc(Cl)c35)C4)C[C@@H]2CC#N)COC1]

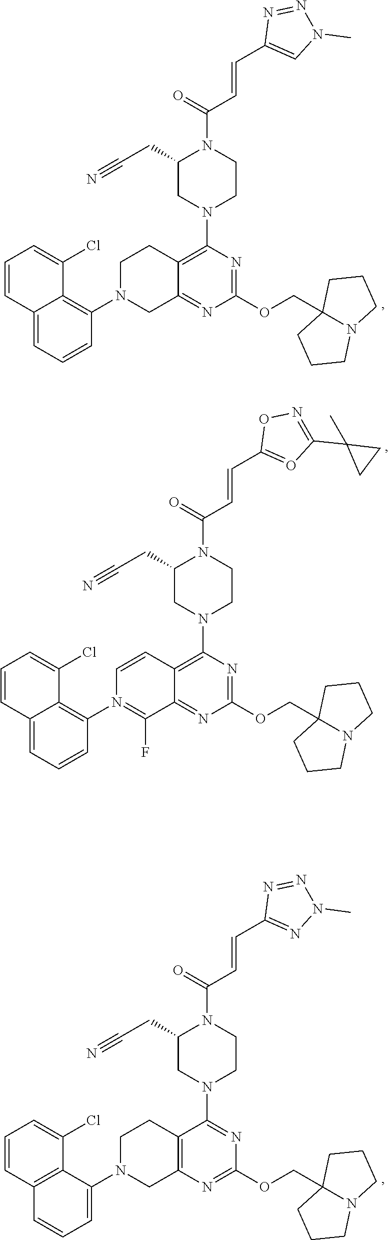

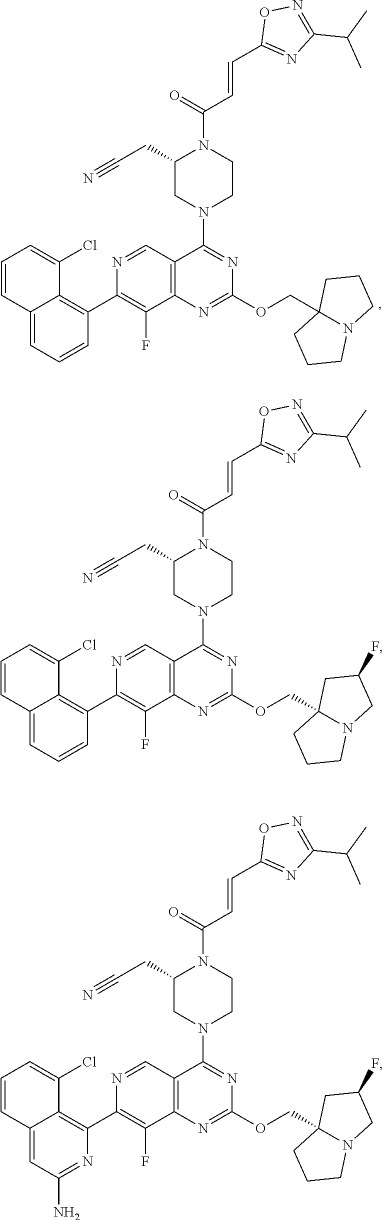

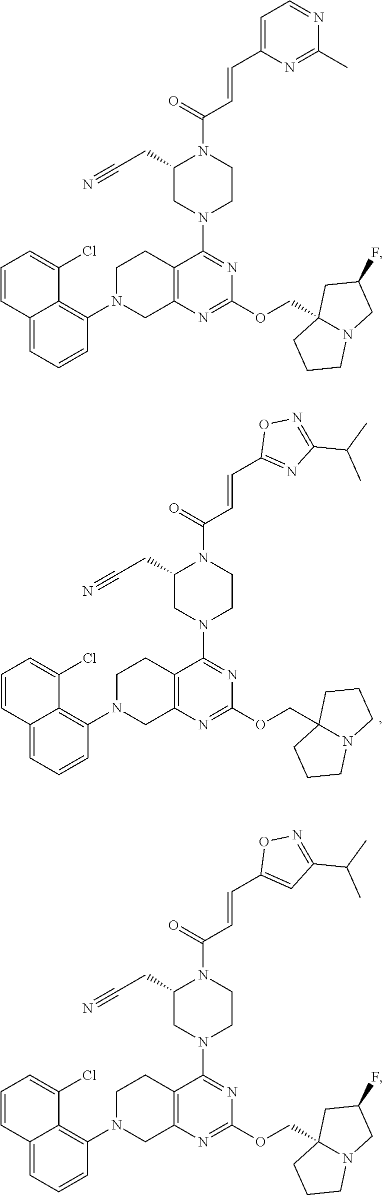 [SMILES: Cc1nccc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1 ; CC(C)c1noc(/C=C/C(=O)N2CCN(c3nc(OCC45CCCN4CCC5)nc4c3CCN(c3cccc5cccc(Cl)c35)C4)C[C@@H]2CC#N)n1 ; CC(C)c1cc(/C=C/C(=O)N2CCN(c3nc(OC[C@@]45CCCN4C[C@H](F)C5)nc4c3CCN(c3cccc5cccc(Cl)c35)C4)C[C@@H]2CC#N)on1]

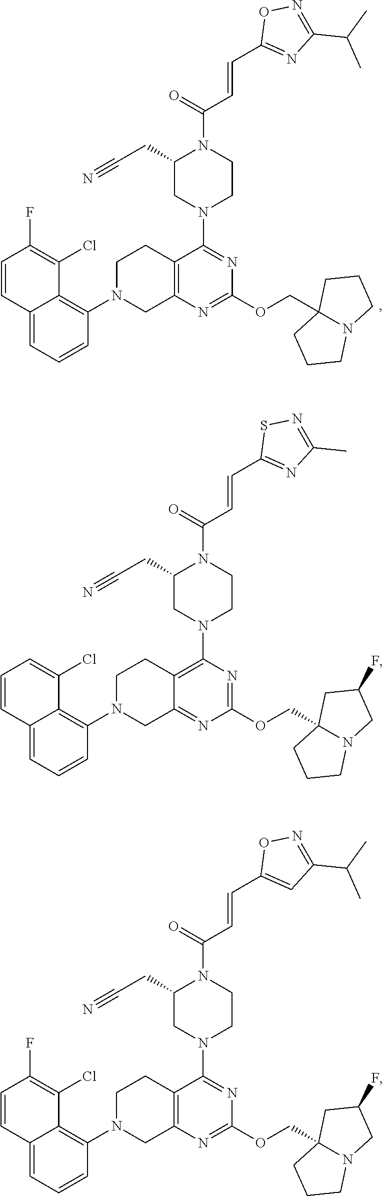 [SMILES: CC(C)c1noc(/C=C/C(=O)N2CCN(c3nc(OCC45CCCN4CCC5)nc4c3CCN(c3cccc5ccc(F)c(Cl)c35)C4)C[C@@H]2CC#N)n1 ; Cc1nsc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1 ; CC(C)c1cc(/C=C/C(=O)N2CCN(c3nc(OC[C@@]45CCCN4C[C@H](F)C5)nc4c3CCN(c3cccc5ccc(F)c(Cl)c35)C4)C[C@@H]2CC#N)on1]

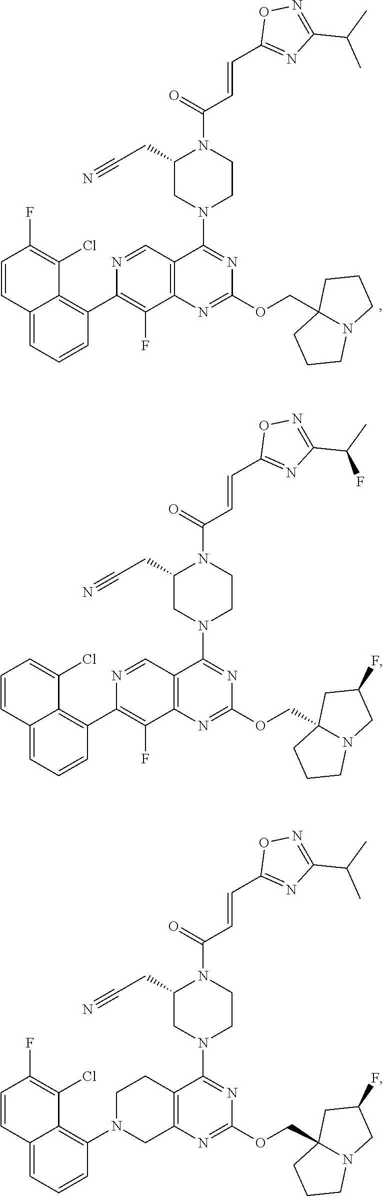 [SMILES: CC(C)c1noc(/C=C/C(=O)N2CCN(c3nc(OCC45CCCN4CCC5)nc4c(F)c(-c5cccc6ccc(F)c(Cl)c56)ncc34)C[C@@H]2CC#N)n1 ; C[C@@H](F)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; CC(C)c1noc(/C=C/C(=O)N2CCN(c3nc(OC[C@]45CCCN4C[C@H](F)C5)nc4c3CCN(c3cccc5ccc(F)c(Cl)c35)C4)C[C@@H]2CC#N)n1]

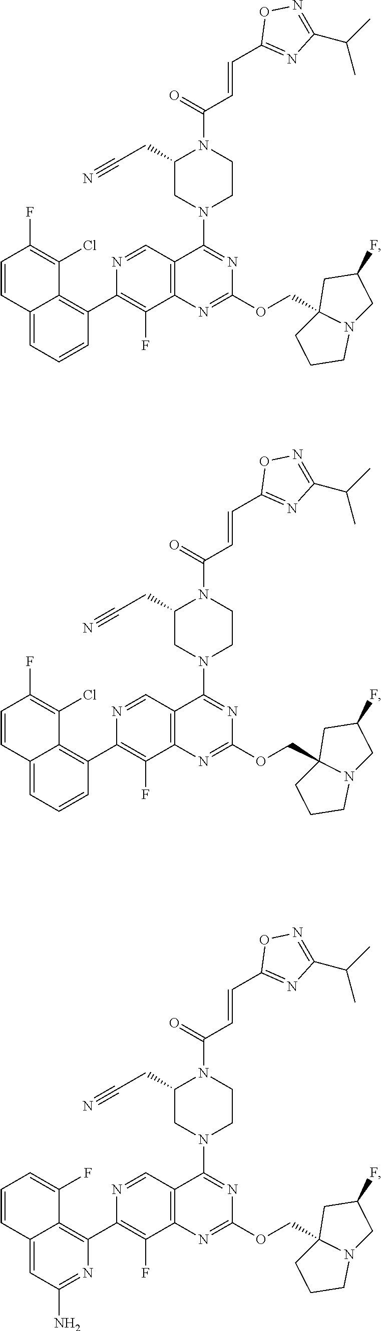 [SMILES: CC(C)c1noc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1 ; CC(C)c1noc(/C=C/C(=O)N2CCN(c3nc(OC[C@]45CCCN4C[C@H](F)C5)nc4c(F)c(-c5cccc6ccc(F)c(Cl)c56)ncc34)C[C@@H]2CC#N)n1 ; CC(C)c1noc(/C=C/C(=O)N2CCN(c3nc(OC[C@@]45CCCN4C[C@H](F)C5)nc4c(F)c(-c5nc(N)cc6cccc(F)c56)ncc34)C[C@@H]2CC#N)n1]

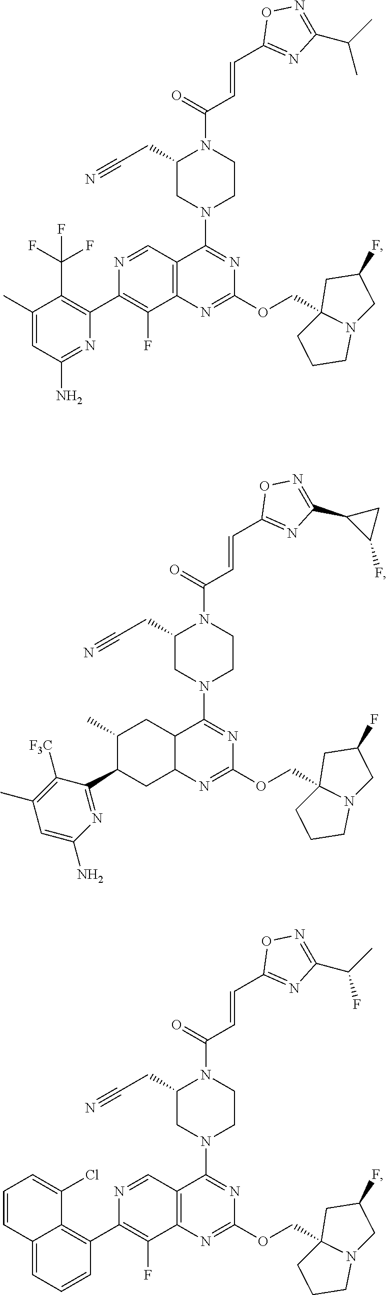 [SMILES: Cc1cc(N)nc(-c2ncc3c(N4CCN(C(=O)/C=C/c5nc(C(C)C)no5)[C@@H](CC#N)C4)nc(OC[C@@]45CCCN4C[C@H](F)C5)nc3c2F)c1C(F)(F)F ; Cc1cc(N)nc([C@@H]2CC3N=C(OC[C@@]45CCCN4C[C@H](F)C5)N=C(N4CCN(C(=O)/C=C/c5nc([C@H]6C[C@@H]6F)no5)[C@@H](CC#N)C4)C3C[C@H]2C)c1C(F)(F)F ; C[C@H](F)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1]

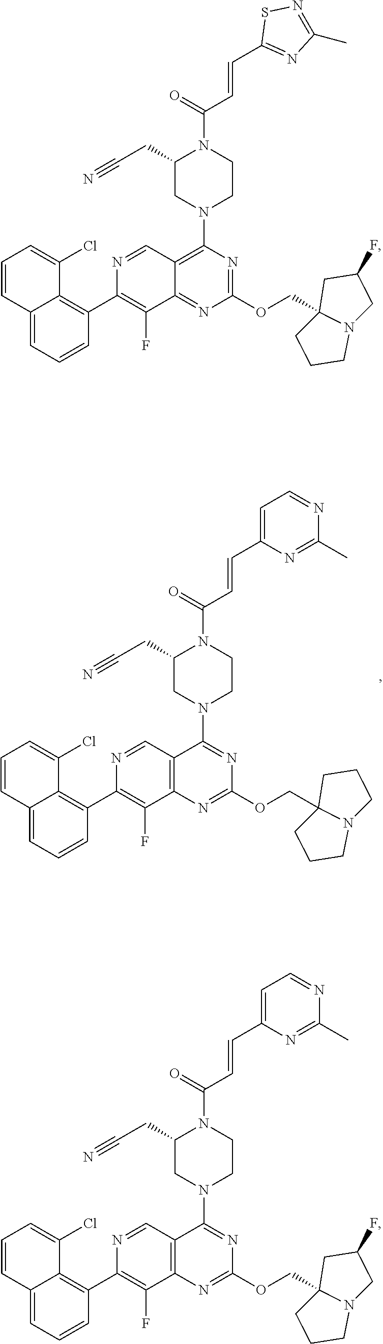 [SMILES: Cc1nsc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; Cc1nccc(/C=C/C(=O)N2CCN(c3nc(OCC45CCCN4CCC5)nc4c(F)c(-c5cccc6cccc(Cl)c56)ncc34)C[C@@H]2CC#N)n1 ; Cc1nccc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1]

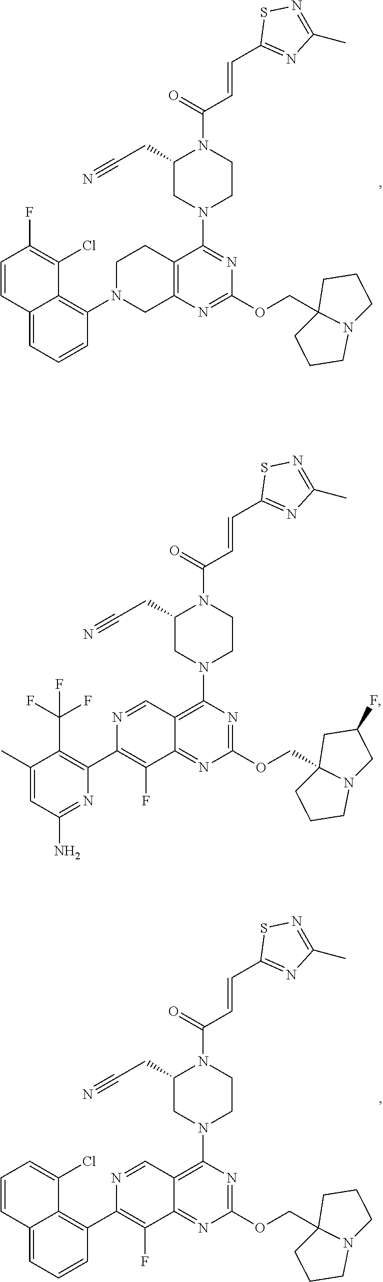 [SMILES: Cc1nsc(/C=C/C(=O)N2CCN(c3nc(OCC45CCCN4CCC5)nc4c3CCN(c3cccc5ccc(F)c(Cl)c35)C4)C[C@@H]2CC#N)n1 ; Cc1nsc(/C=C/C(=O)N2CCN(c3nc(OC[C@@]45CCCN4C[C@H](F)C5)nc4c(F)c(-c5nc(N)cc(C)c5C(F)(F)F)ncc34)C[C@@H]2CC#N)n1 ; Cc1nsc(/C=C/C(=O)N2CCN(c3nc(OCC45CCCN4CCC5)nc4c(F)c(-c5cccc6cccc(Cl)c56)ncc34)C[C@@H]2CC#N)n1]

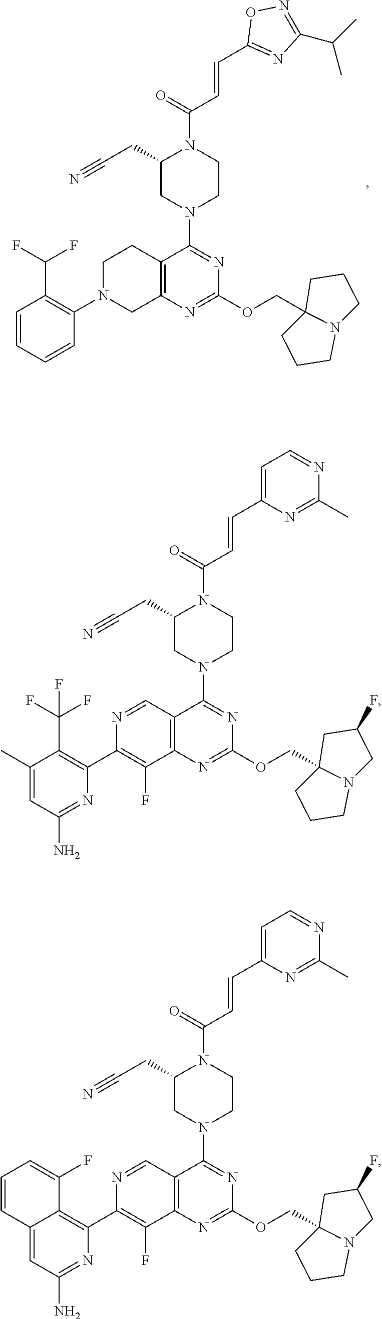 [SMILES: CC(C)c1noc(/C=C/C(=O)N2CCN(c3nc(OCC45CCCN4CCC5)nc4c3CCN(c3ccccc3C(F)F)C4)C[C@@H]2CC#N)n1 ; Cc1nccc(/C=C/C(=O)N2CCN(c3nc(OC[C@@]45CCCN4C[C@H](F)C5)nc4c(F)c(-c5nc(N)cc(C)c5C(F)(F)F)ncc34)C[C@@H]2CC#N)n1 ; Cc1nccc(/C=C/C(=O)N2CCN(c3nc(OC[C@@]45CCCN4C[C@H](F)C5)nc4c(F)c(-c5nc(N)cc6cccc(F)c56)ncc34)C[C@@H]2CC#N)n1]

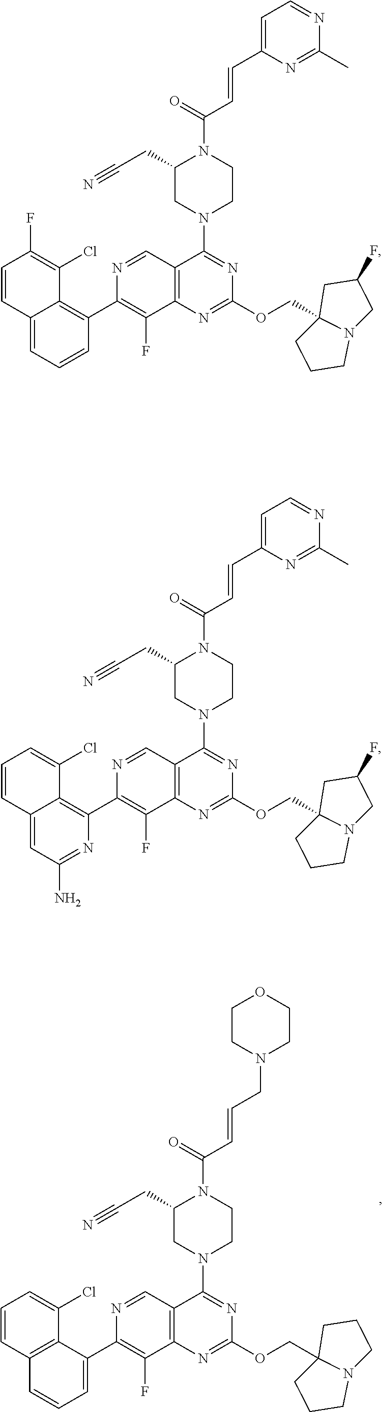 [SMILES: Cc1nccc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1 ; Cc1nccc(/C=C/C(=O)N2CCN(c3nc(OC[C@@]45CCCN4C[C@H](F)C5)nc4c(F)c(-c5nc(N)cc6cccc(Cl)c56)ncc34)C[C@@H]2CC#N)n1 ; N#CC[C@H]1CN(c2nc(OCC34CCCN3CCC4)nc3c(F)c(-c4cccc5cccc(Cl)c45)ncc23)CCN1C(=O)/C=C/CN1CCOCC1]

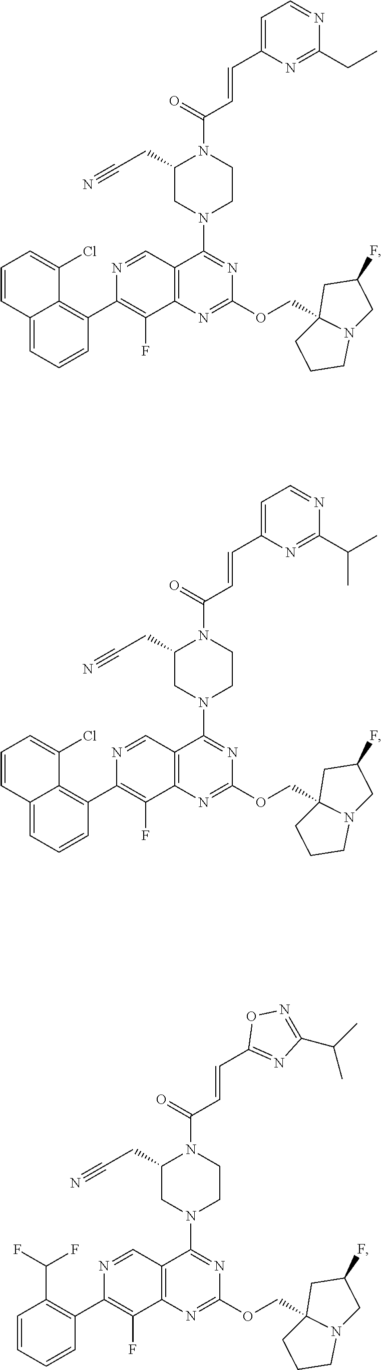 [SMILES: CCc1nccc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; CC(C)c1nccc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; CC(C)c1noc(/C=C/C(=O)N2CCN(c3nc(OC[C@@]45CCCN4C[C@H](F)C5)nc4c(F)c(-c5ccccc5C(F)F)ncc34)C[C@@H]2CC#N)n1]

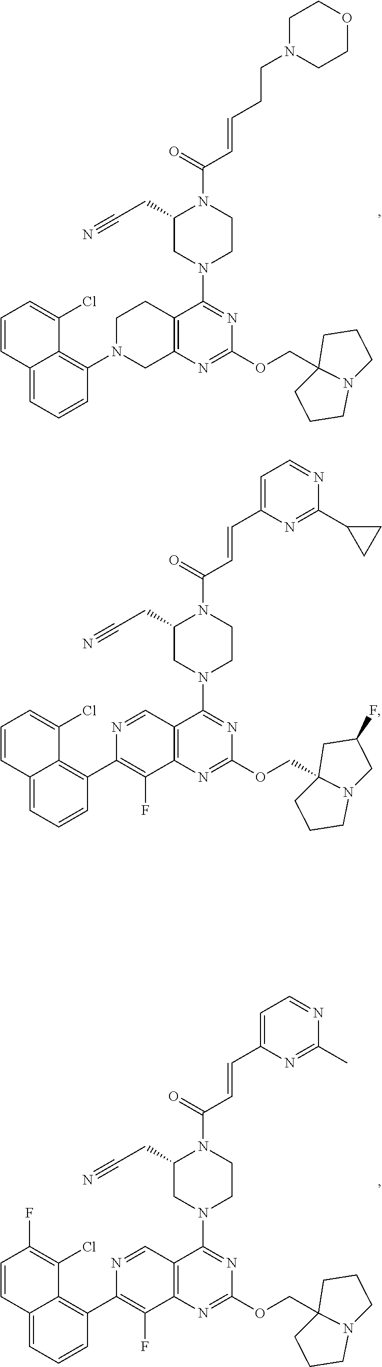

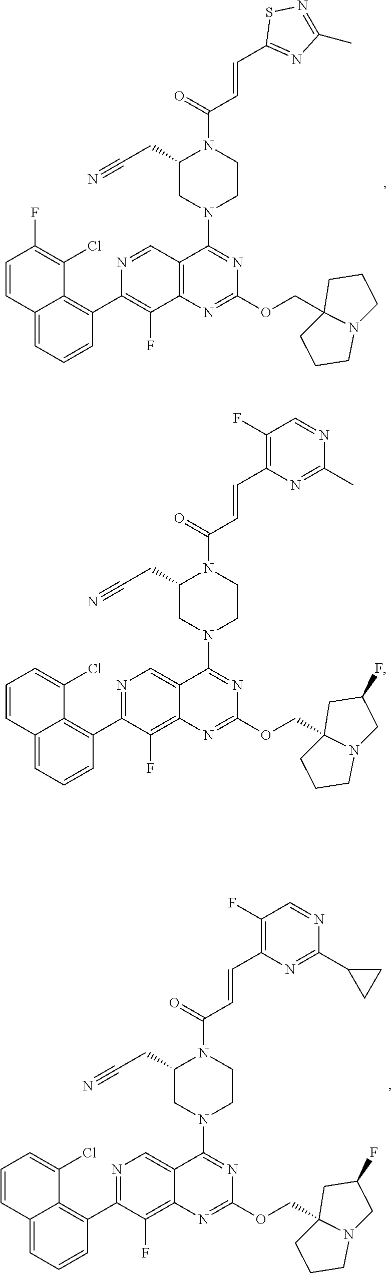 [SMILES: Cc1nsc(/C=C/C(=O)N2CCN(c3nc(OCC45CCCN4CCC5)nc4c(F)c(-c5cccc6ccc(F)c(Cl)c56)ncc34)C[C@@H]2CC#N)n1 ; Cc1ncc(F)c(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/c1nc(C2CC2)ncc1F]

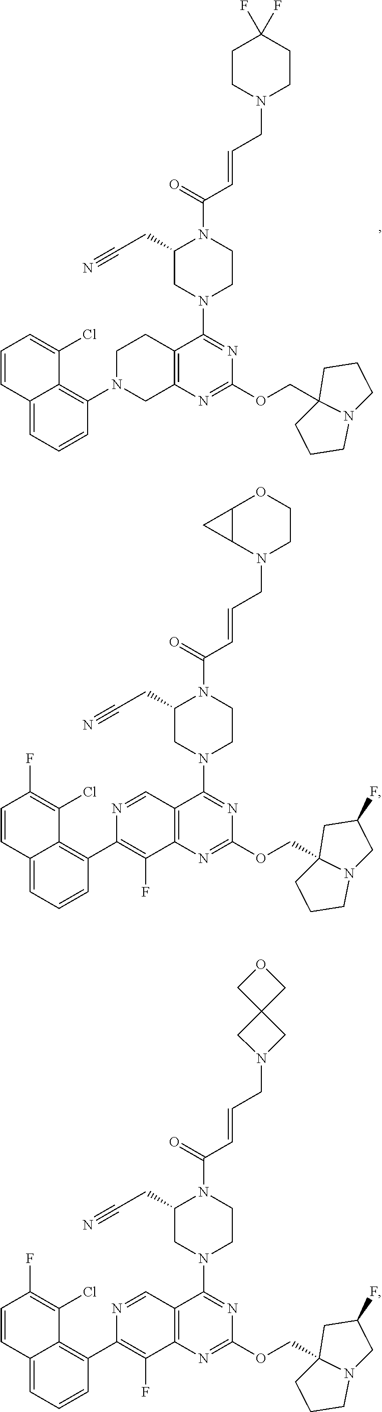 [SMILES: N#CC[C@H]1CN(c2nc(OCC34CCCN3CCC4)nc3c2CCN(c2cccc4cccc(Cl)c24)C3)CCN1C(=O)/C=C/CN1CCC(F)(F)CC1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCOC2CC21 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2(COC2)C1]

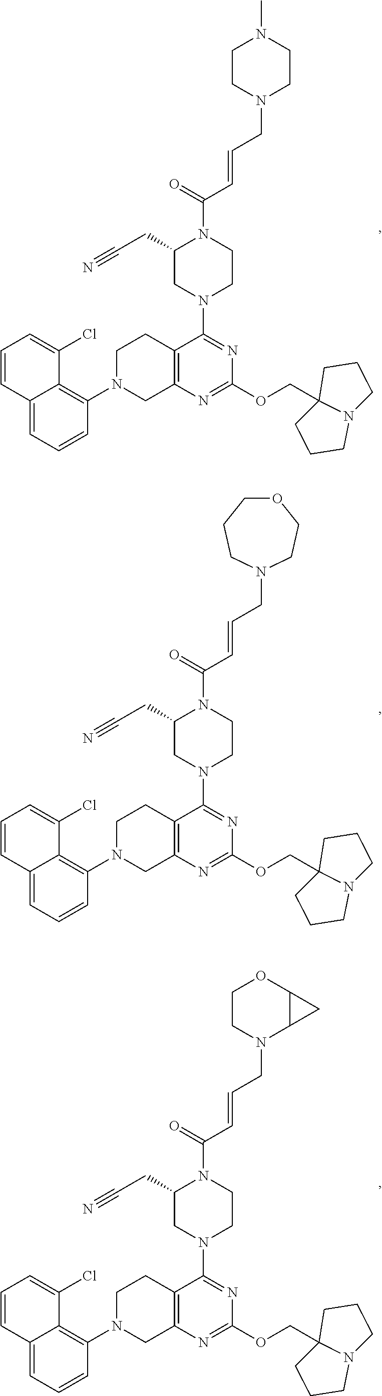 [SMILES: CN1CCN(C/C=C/C(=O)N2CCN(c3nc(OCC45CCCN4CCC5)nc4c3CCN(c3cccc5cccc(Cl)c35)C4)C[C@@H]2CC#N)CC1 ; N#CC[C@H]1CN(c2nc(OCC34CCCN3CCC4)nc3c2CCN(c2cccc4cccc(Cl)c24)C3)CCN1C(=O)/C=C/CN1CCCOCC1 ; N#CC[C@H]1CN(c2nc(OCC34CCCN3CCC4)nc3c2CCN(c2cccc4cccc(Cl)c24)C3)CCN1C(=O)/C=C/CN1CCOC2CC21]

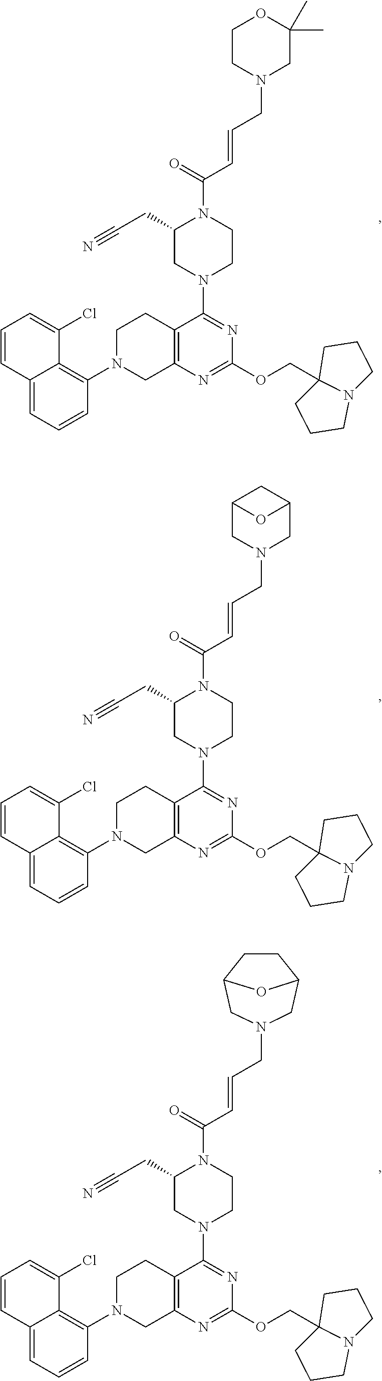 [SMILES: CC1(C)CN(C/C=C/C(=O)N2CCN(c3nc(OCC45CCCN4CCC5)nc4c3CCN(c3cccc5cccc(Cl)c35)C4)C[C@@H]2CC#N)CCO1 ; N#CC[C@H]1CN(c2nc(OCC34CCCN3CCC4)nc3c2CCN(c2cccc4cccc(Cl)c24)C3)CCN1C(=O)/C=C/CN1CC2CC(C1)O2 ; N#CC[C@H]1CN(c2nc(OCC34CCCN3CCC4)nc3c2CCN(c2cccc4cccc(Cl)c24)C3)CCN1C(=O)/C=C/CN1CC2CCC(C1)O2]

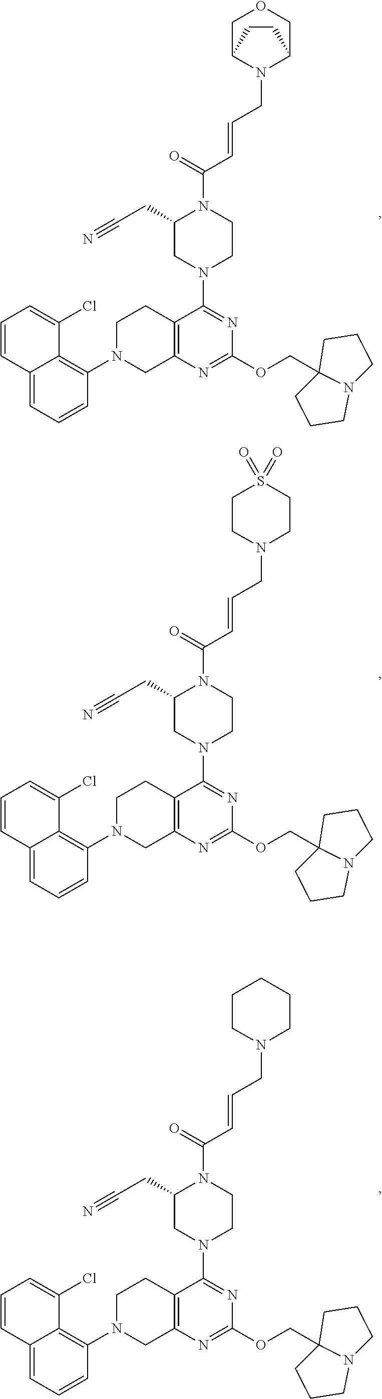

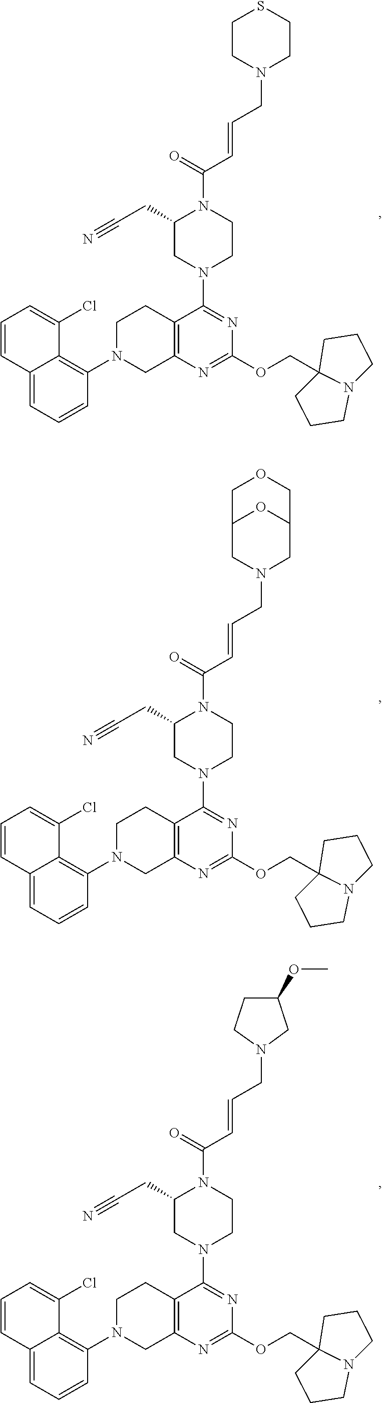

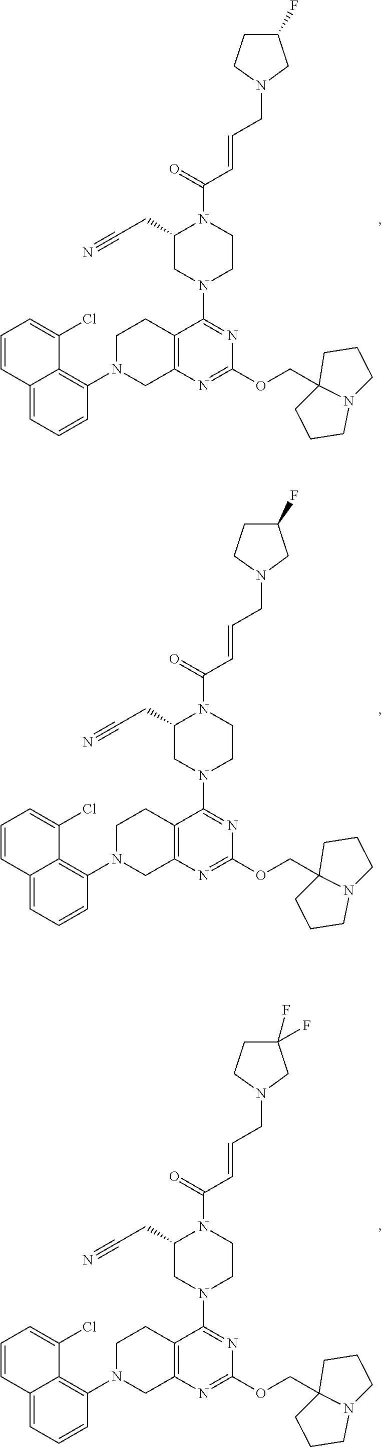 [SMILES: N#CC[C@H]1CN(c2nc(OCC34CCCN3CCC4)nc3c2CCN(c2cccc4cccc(Cl)c24)C3)CCN1C(=O)/C=C/CN1CC[C@H](F)C1 ; N#CC[C@H]1CN(c2nc(OCC34CCCN3CCC4)nc3c2CCN(c2cccc4cccc(Cl)c24)C3)CCN1C(=O)/C=C/CN1CC[C@@H](F)C1 ; N#CC[C@H]1CN(c2nc(OCC34CCCN3CCC4)nc3c2CCN(c2cccc4cccc(Cl)c24)C3)CCN1C(=O)/C=C/CN1CCC(F)(F)C1]

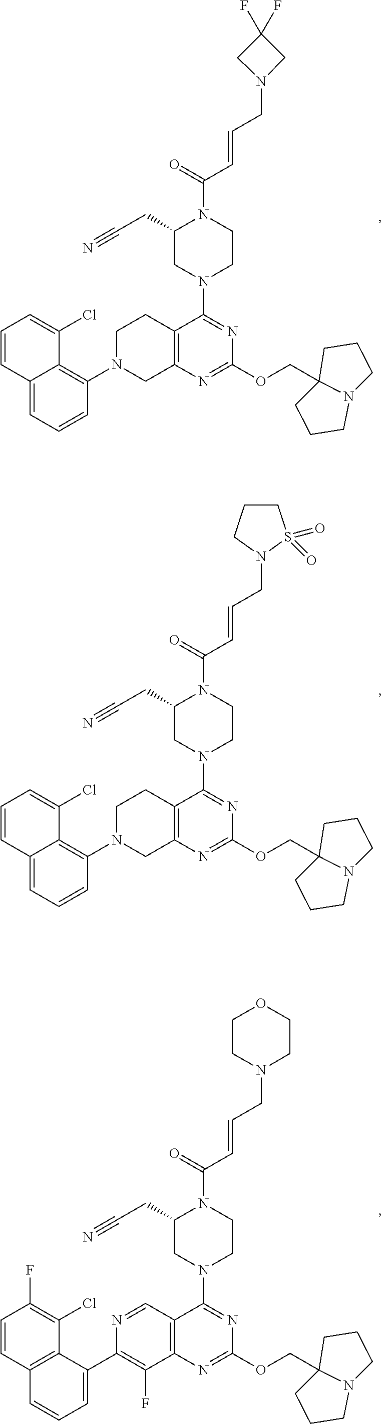 [SMILES: N#CC[C@H]1CN(c2nc(OCC34CCCN3CCC4)nc3c2CCN(c2cccc4cccc(Cl)c24)C3)CCN1C(=O)/C=C/CN1CC(F)(F)C1 ; N#CC[C@H]1CN(c2nc(OCC34CCCN3CCC4)nc3c2CCN(c2cccc4cccc(Cl)c24)C3)CCN1C(=O)/C=C/CN1CCCS1(=O)=O ; N#CC[C@H]1CN(c2nc(OCC34CCCN3CCC4)nc3c(F)c(-c4cccc5ccc(F)c(Cl)c45)ncc23)CCN1C(=O)/C=C/CN1CCOCC1]

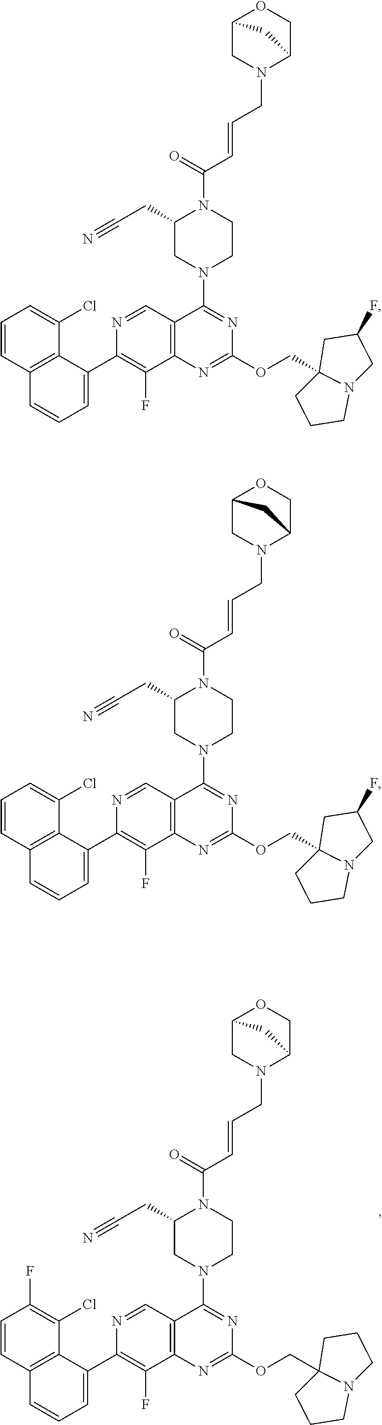 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@@H]2C[C@H]1CO2 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@H]2C[C@@H]1CO2 ; N#CC[C@H]1CN(c2nc(OCC34CCCN3CCC4)nc3c(F)c(-c4cccc5ccc(F)c(Cl)c45)ncc23)CCN1C(=O)/C=C/CN1C[C@@H]2C[C@H]1CO2]

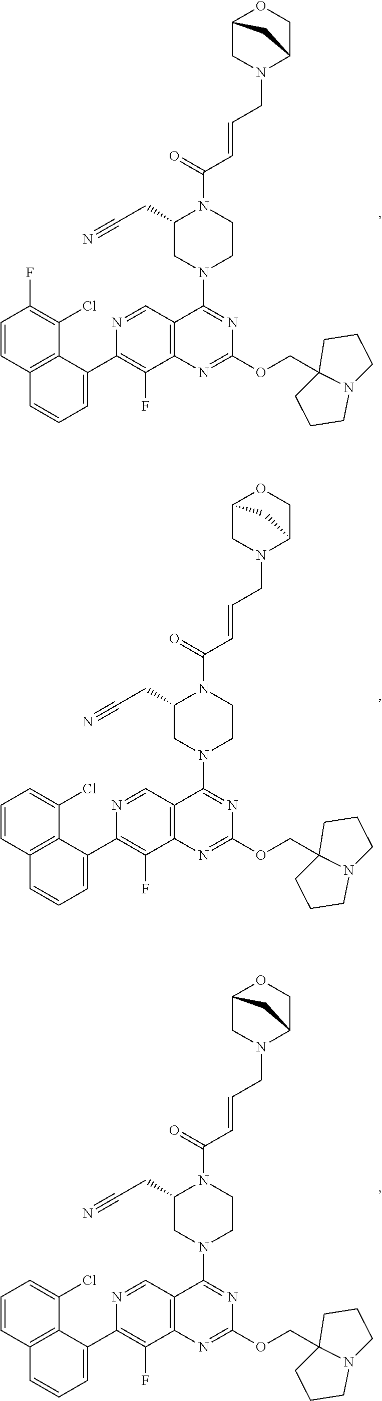 [SMILES: N#CC[C@H]1CN(c2nc(OCC34CCCN3CCC4)nc3c(F)c(-c4cccc5ccc(F)c(Cl)c45)ncc23)CCN1C(=O)/C=C/CN1C[C@H]2C[C@@H]1CO2 ; N#CC[C@H]1CN(c2nc(OCC34CCCN3CCC4)nc3c(F)c(-c4cccc5cccc(Cl)c45)ncc23)CCN1C(=O)/C=C/CN1C[C@@H]2C[C@H]1CO2 ; N#CC[C@H]1CN(c2nc(OCC34CCCN3CCC4)nc3c(F)c(-c4cccc5cccc(Cl)c45)ncc23)CCN1C(=O)/C=C/CN1C[C@H]2C[C@@H]1CO2]

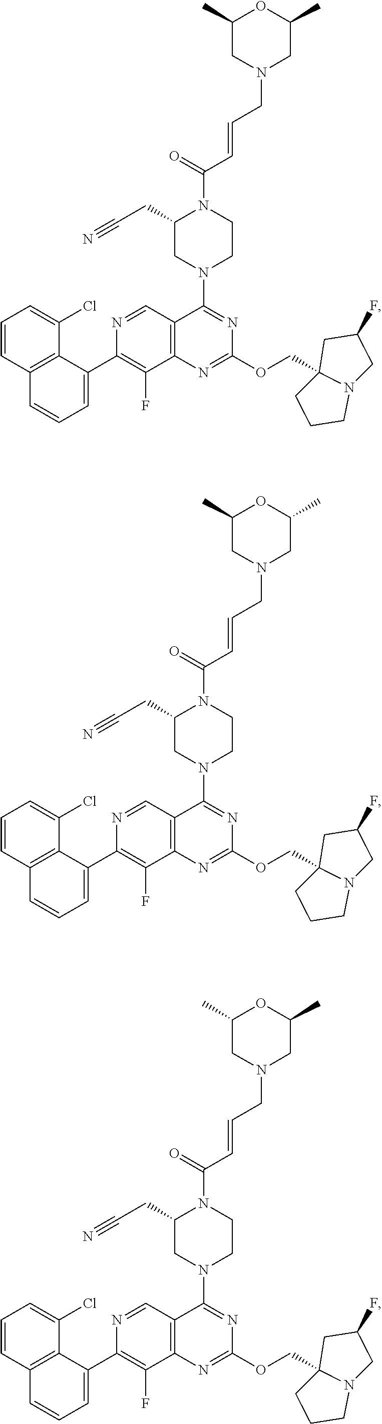 [SMILES: C[C@@H]1CN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C[C@H](C)O1 ; C[C@@H]1CN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C[C@@H](C)O1 ; C[C@H]1CN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C[C@H](C)O1]

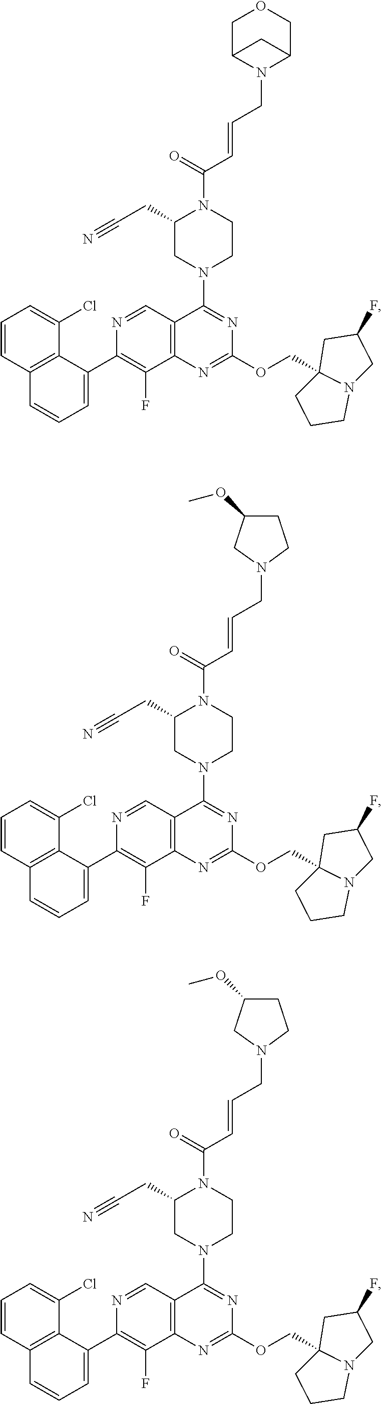 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C2COCC1C2 ; CO[C@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1 ; CO[C@@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1]

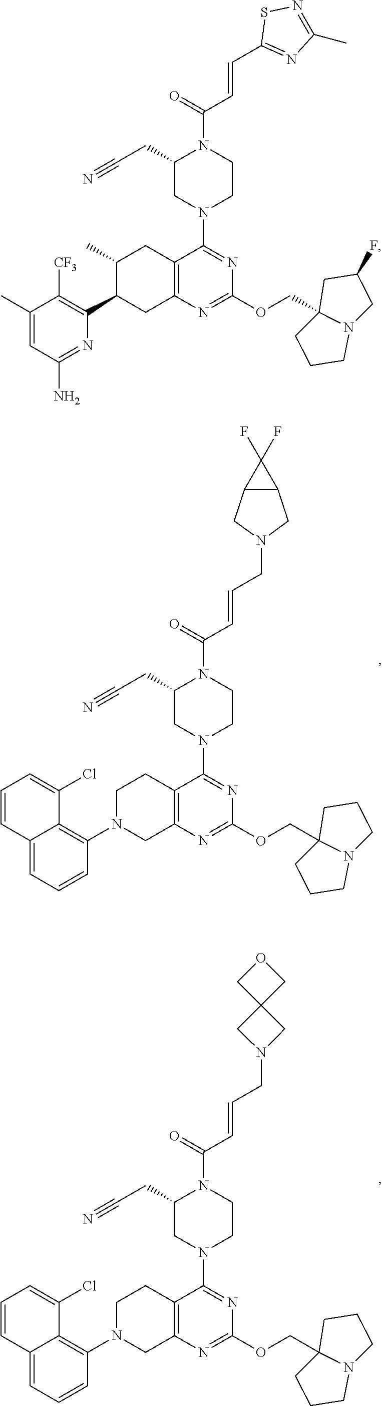 [SMILES: Cc1nsc(/C=C/C(=O)N2CCN(c3nc(OC[C@@]45CCCN4C[C@H](F)C5)nc4c3C[C@@H](C)[C@H](c3nc(N)cc(C)c3C(F)(F)F)C4)C[C@@H]2CC#N)n1 ; N#CC[C@H]1CN(c2nc(OCC34CCCN3CCC4)nc3c2CCN(c2cccc4cccc(Cl)c24)C3)CCN1C(=O)/C=C/CN1CC2C(C1)C2(F)F ; N#CC[C@H]1CN(c2nc(OCC34CCCN3CCC4)nc3c2CCN(c2cccc4cccc(Cl)c24)C3)CCN1C(=O)/C=C/CN1CC2(COC2)C1]

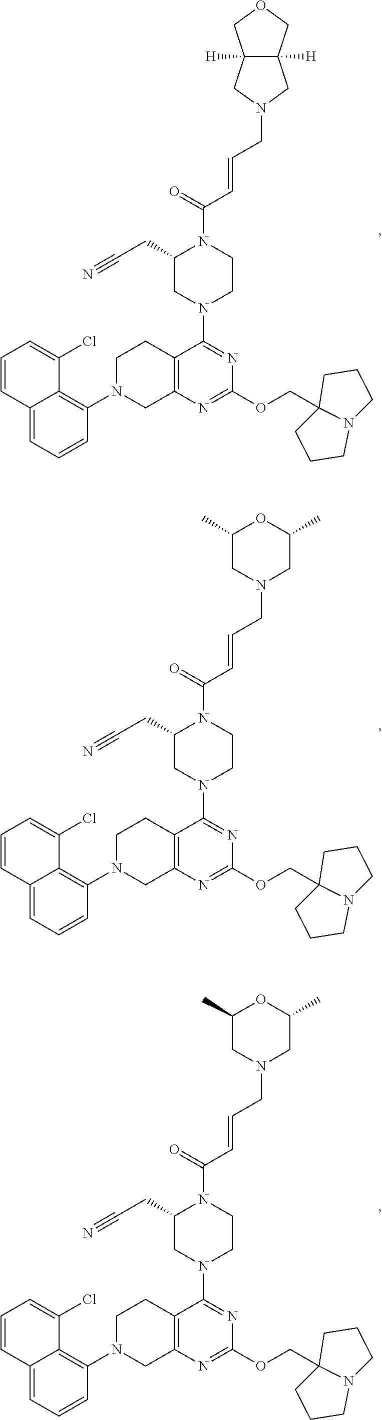 [SMILES: N#CC[C@H]1CN(c2nc(OCC34CCCN3CCC4)nc3c2CCN(c2cccc4cccc(Cl)c24)C3)CCN1C(=O)/C=C/CN1C[C@H]2COC[C@H]2C1 ; C[C@@H]1CN(C/C=C/C(=O)N2CCN(c3nc(OCC45CCCN4CCC5)nc4c3CCN(c3cccc5cccc(Cl)c35)C4)C[C@@H]2CC#N)C[C@H](C)O1 ; C[C@@H]1CN(C/C=C/C(=O)N2CCN(c3nc(OCC45CCCN4CCC5)nc4c3CCN(c3cccc5cccc(Cl)c35)C4)C[C@@H]2CC#N)C[C@@H](C)O1]

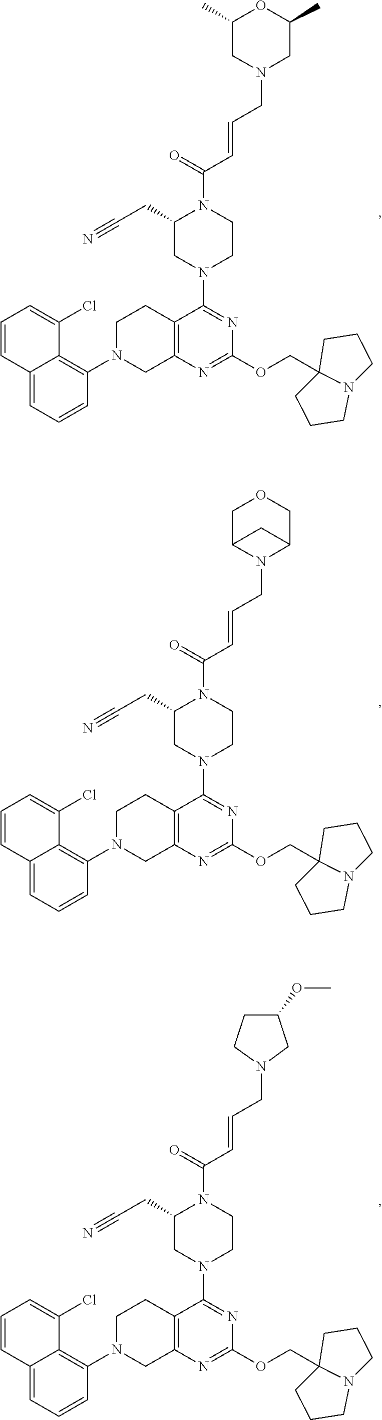 [SMILES: C[C@H]1CN(C/C=C/C(=O)N2CCN(c3nc(OCC45CCCN4CCC5)nc4c3CCN(c3cccc5cccc(Cl)c35)C4)C[C@@H]2CC#N)C[C@H](C)O1 ; N#CC[C@H]1CN(c2nc(OCC34CCCN3CCC4)nc3c2CCN(c2cccc4cccc(Cl)c24)C3)CCN1C(=O)/C=C/CN1C2COCC1C2 ; CO[C@H]1CCN(C/C=C/C(=O)N2CCN(c3nc(OCC45CCCN4CCC5)nc4c3CCN(c3cccc5cccc(Cl)c35)C4)C[C@@H]2CC#N)C1]

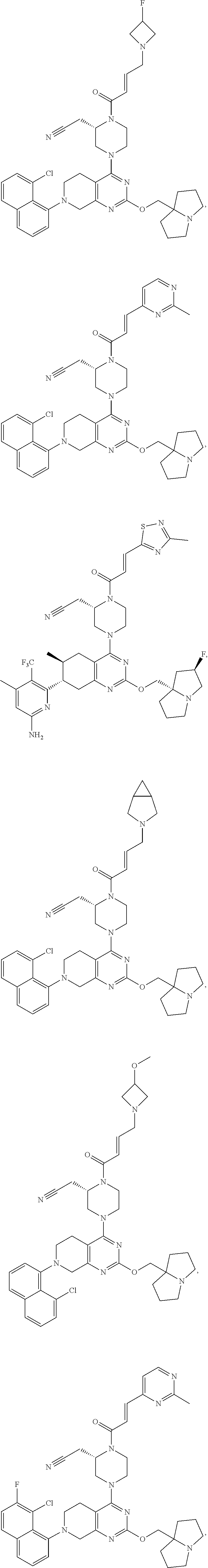

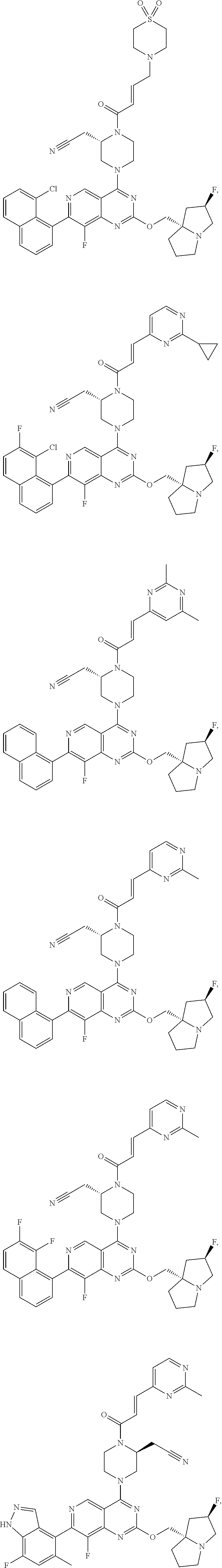

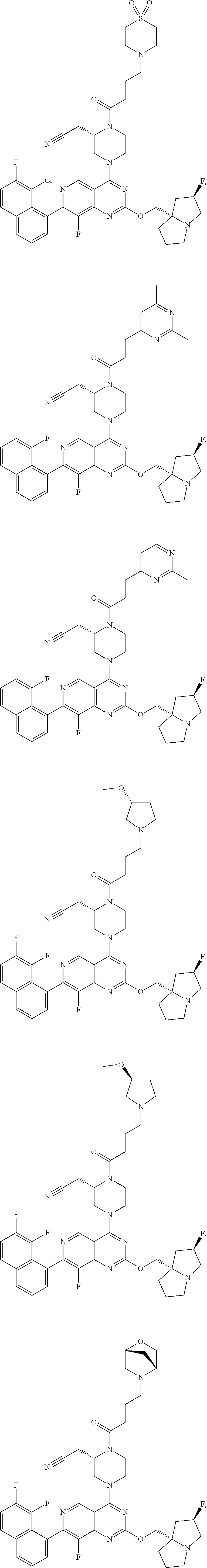

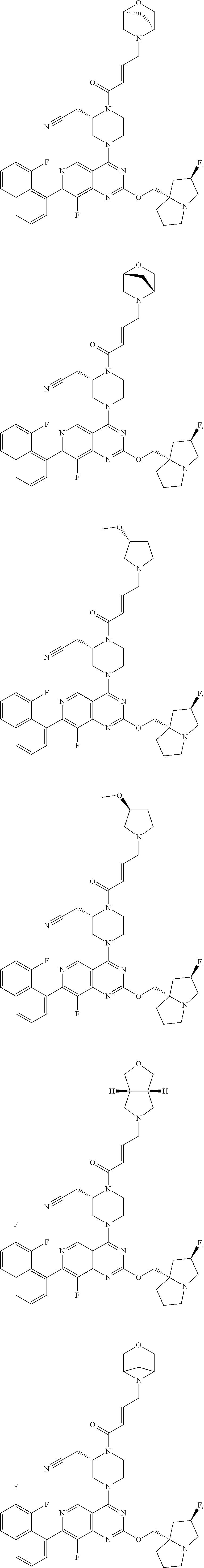

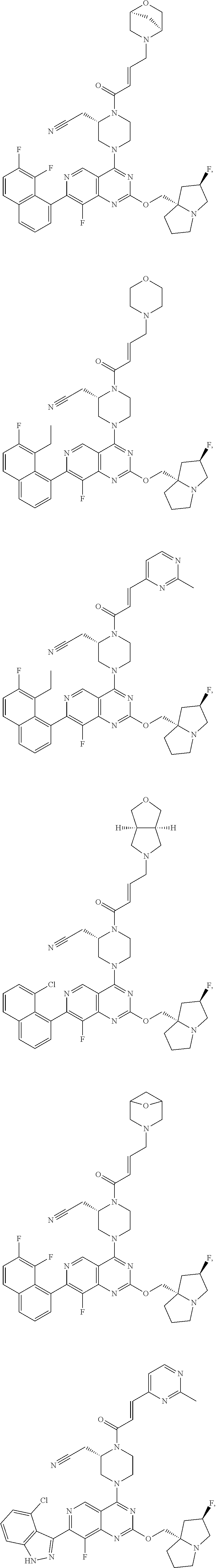

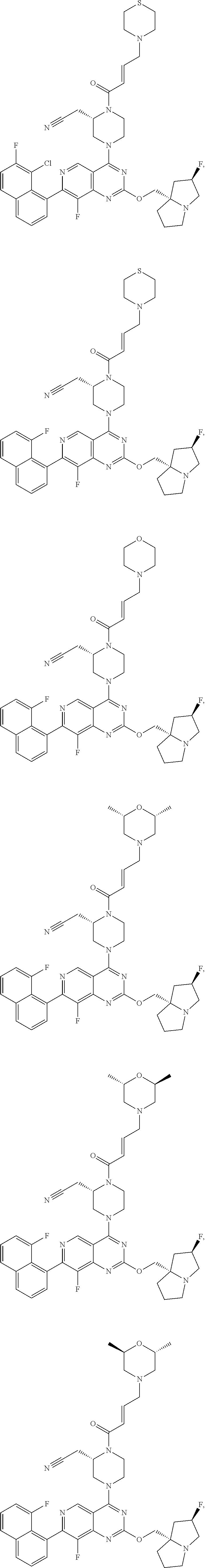

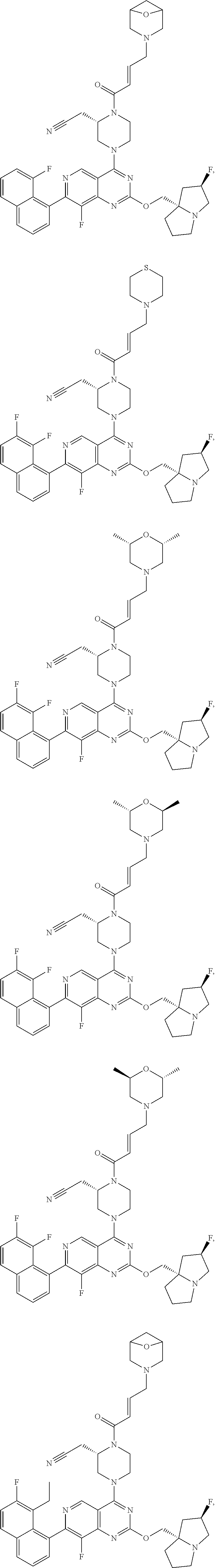

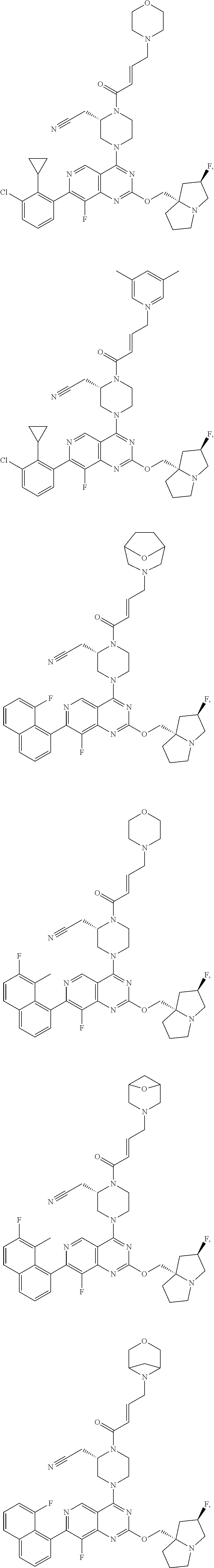

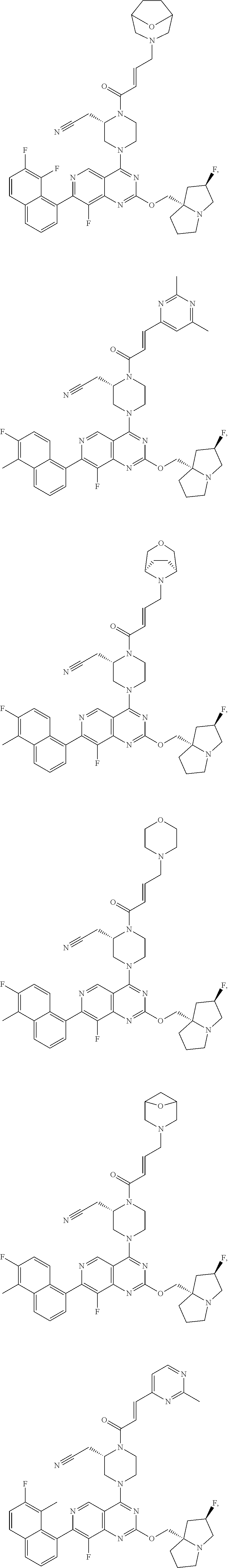

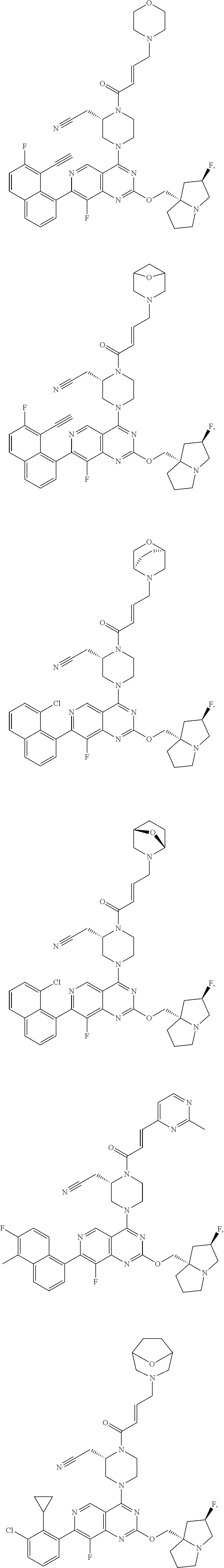

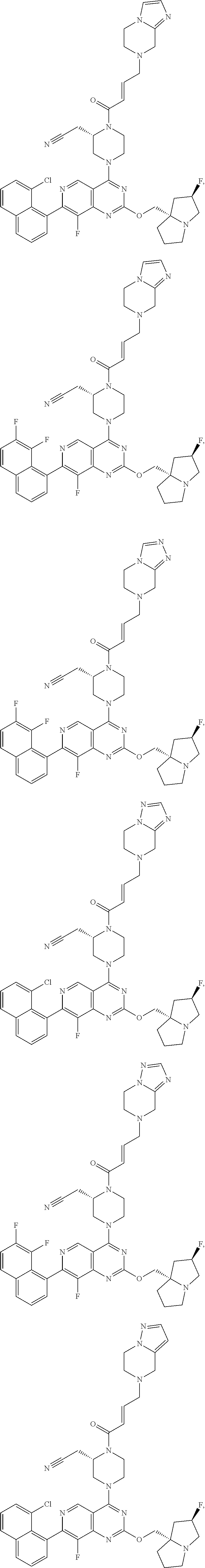

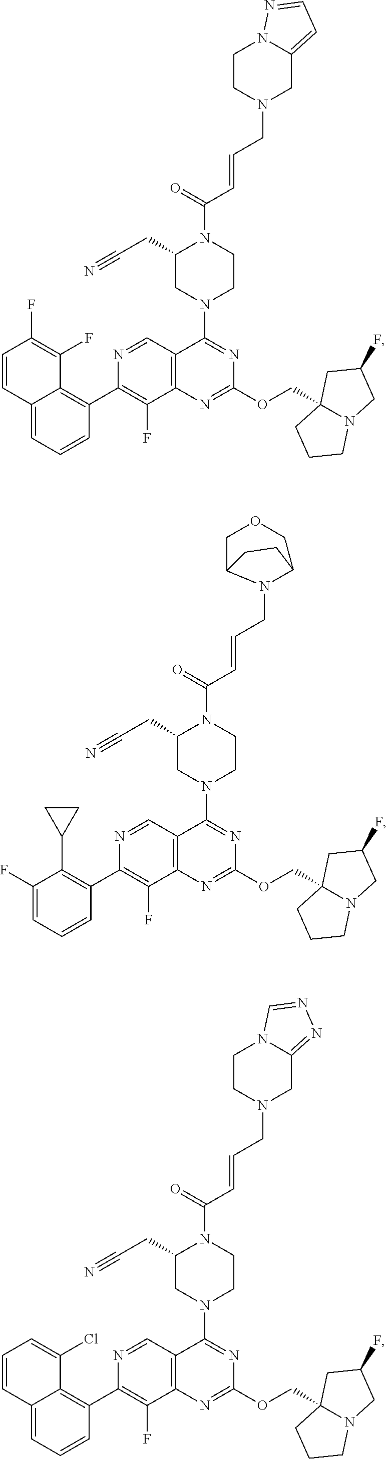 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCn2nccc2C1 ; N#CC[C@H]1CN(c2nc(OC[C@@]34CCCN3C[C@H](F)C4)nc3c(F)c(-c4cccc(F)c4C4CC4)ncc23)CCN1C(=O)/C=C/CN1C2CCC1COC2 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCn2cnnc2C1]

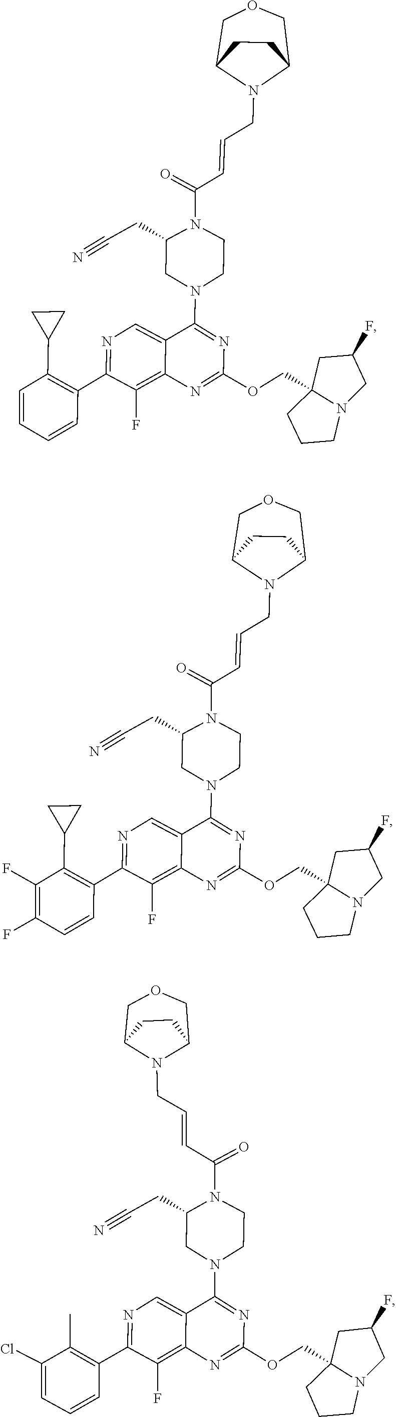 [SMILES: N#CC[C@H]1CN(c2nc(OC[C@@]34CCCN3C[C@H](F)C4)nc3c(F)c(-c4ccccc4C4CC4)ncc23)CCN1C(=O)/C=C/CN1[C@@H]2CC[C@H]1COC2 ; N#CC[C@H]1CN(c2nc(OC[C@@]34CCCN3C[C@H](F)C4)nc3c(F)c(-c4ccc(F)c(F)c4C4CC4)ncc23)CCN1C(=O)/C=C/CN1[C@@H]2CC[C@H]1COC2 ; Cc1c(Cl)cccc1-c1ncc2c(N3CCN(C(=O)/C=C/CN4[C@@H]5CC[C@H]4COC5)[C@@H](CC#N)C3)nc(OC[C@@]34CCCN3C[C@H](F)C4)nc2c1F]

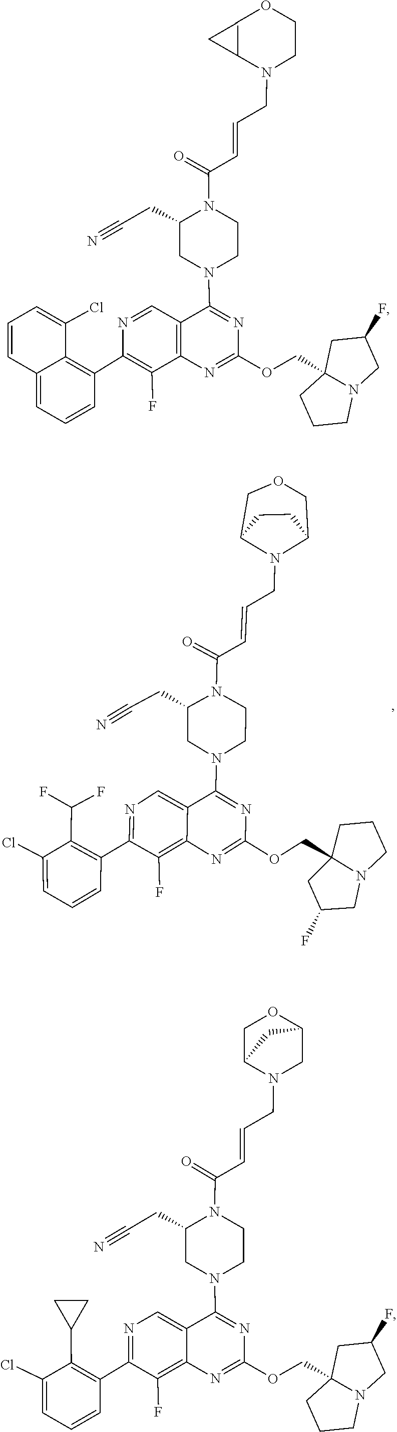

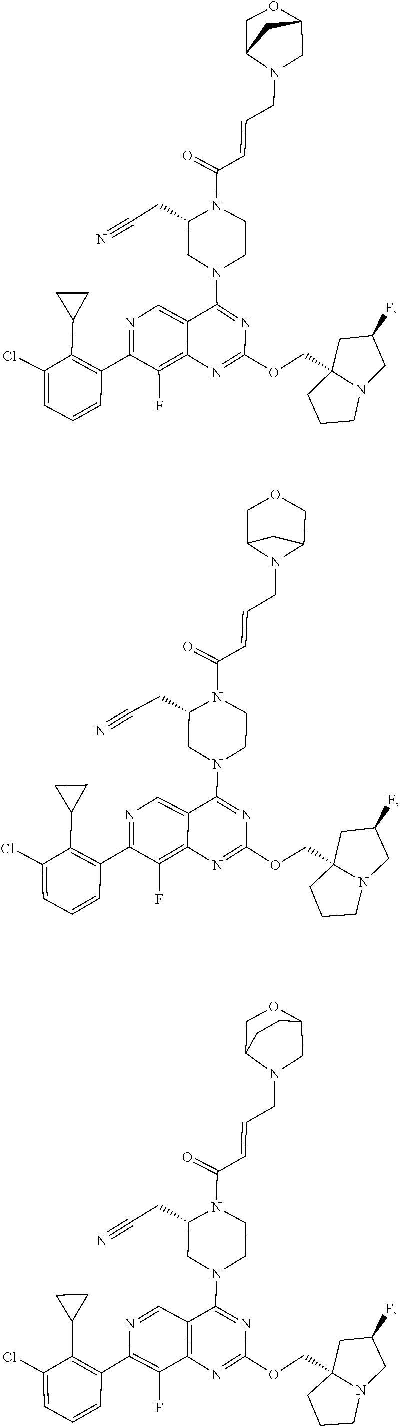 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1C[C@@H]2C[C@H]1CO2 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1C2COCC1C2 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CC2CCC1CO2]

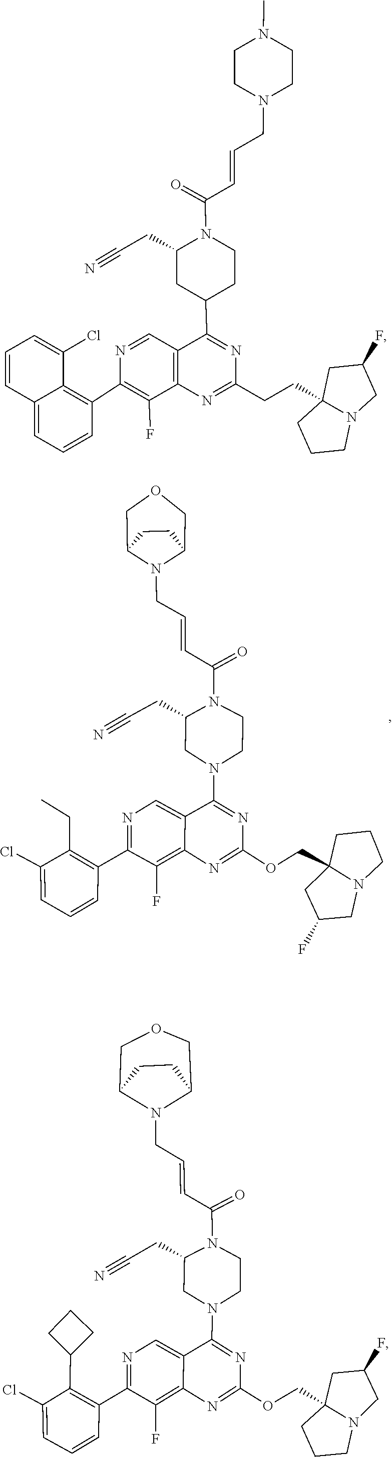 [SMILES: CN1CCN(C/C=C/C(=O)N2CCC(c3nc(CC[C@@]45CCCN4C[C@H](F)C5)nc4c(F)c(-c5cccc6cccc(Cl)c56)ncc34)C[C@@H]2CC#N)CC1 ; CCc1c(Cl)cccc1-c1ncc2c(N3CCN(C(=O)/C=C/CN4[C@@H]5CC[C@H]4COC5)[C@@H](CC#N)C3)nc(OC[C@@]34CCCN3C[C@H](F)C4)nc2c1F ; N#CC[C@H]1CN(c2nc(OC[C@@]34CCCN3C[C@H](F)C4)nc3c(F)c(-c4cccc(Cl)c4C4CCC4)ncc23)CCN1C(=O)/C=C/CN1[C@@H]2CC[C@H]1COC2]

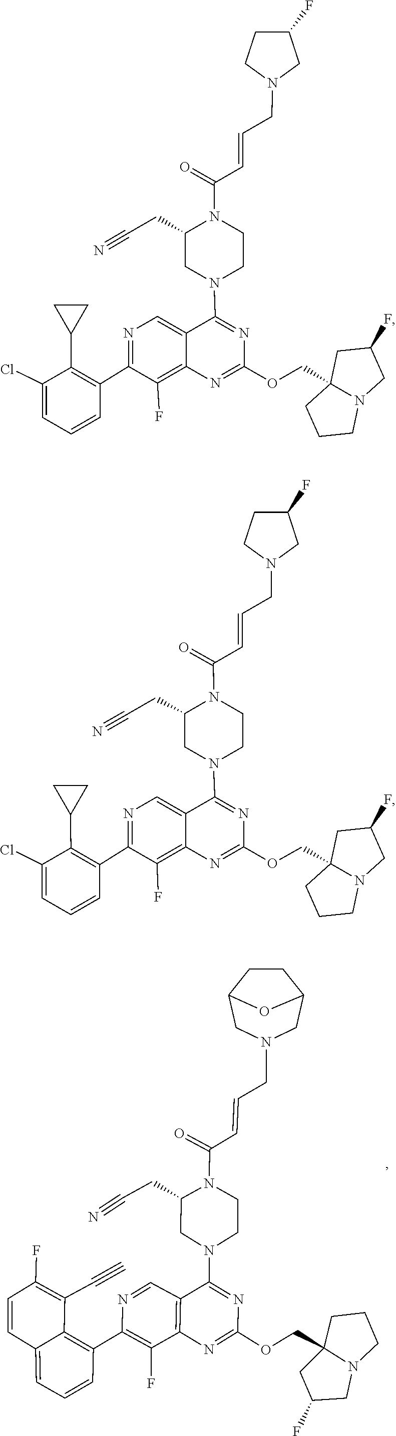 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CC[C@H](F)C1 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CC[C@@H](F)C1 ; C#Cc1c(F)ccc2cccc(-c3ncc4c(N5CCN(C(=O)/C=C/CN6CC7CCC(C6)O7)[C@@H](CC#N)C5)nc(OC[C@@]56CCCN5C[C@H](F)C6)nc4c3F)c12]

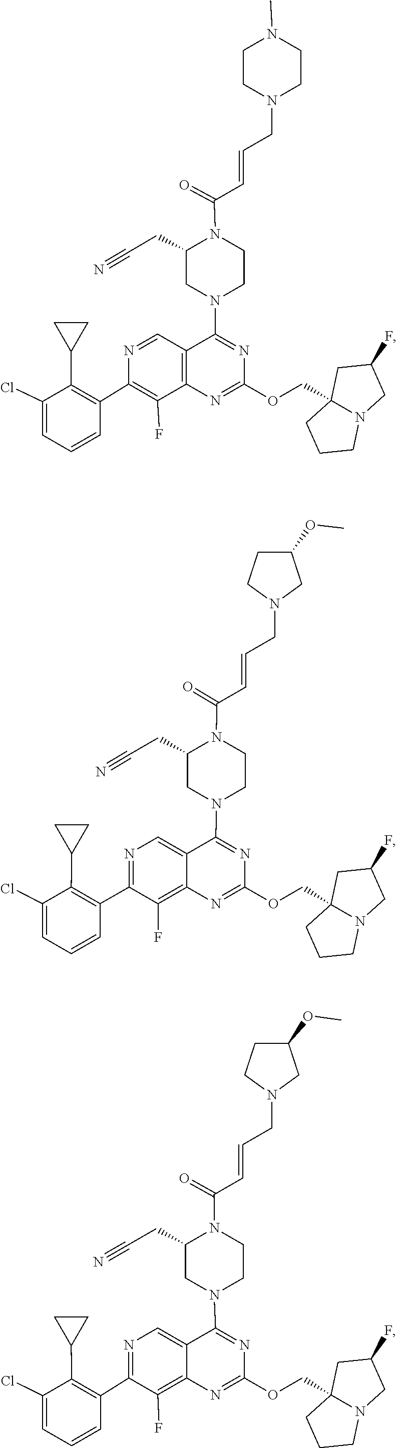 [SMILES: CN1CCN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)CC1 ; CO[C@H]1CCN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)C1 ; CO[C@@H]1CCN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)C1]

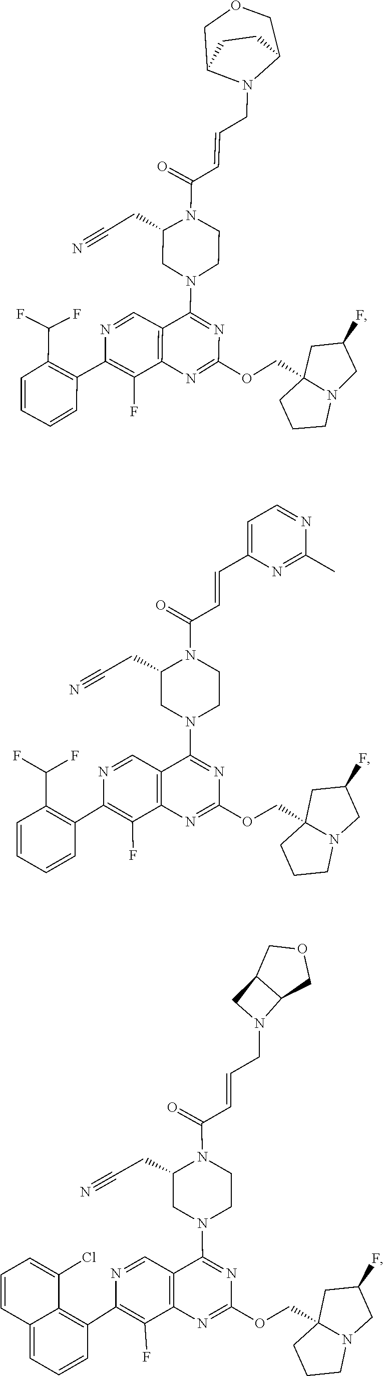 [SMILES: N#CC[C@H]1CN(c2nc(OC[C@@]34CCCN3C[C@H](F)C4)nc3c(F)c(-c4ccccc4C(F)F)ncc23)CCN1C(=O)/C=C/CN1[C@@H]2CC[C@H]1COC2 ; Cc1nccc(/C=C/C(=O)N2CCN(c3nc(OC[C@@]45CCCN4C[C@H](F)C5)nc4c(F)c(-c5ccccc5C(F)F)ncc34)C[C@@H]2CC#N)n1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@H]2COC[C@H]21]

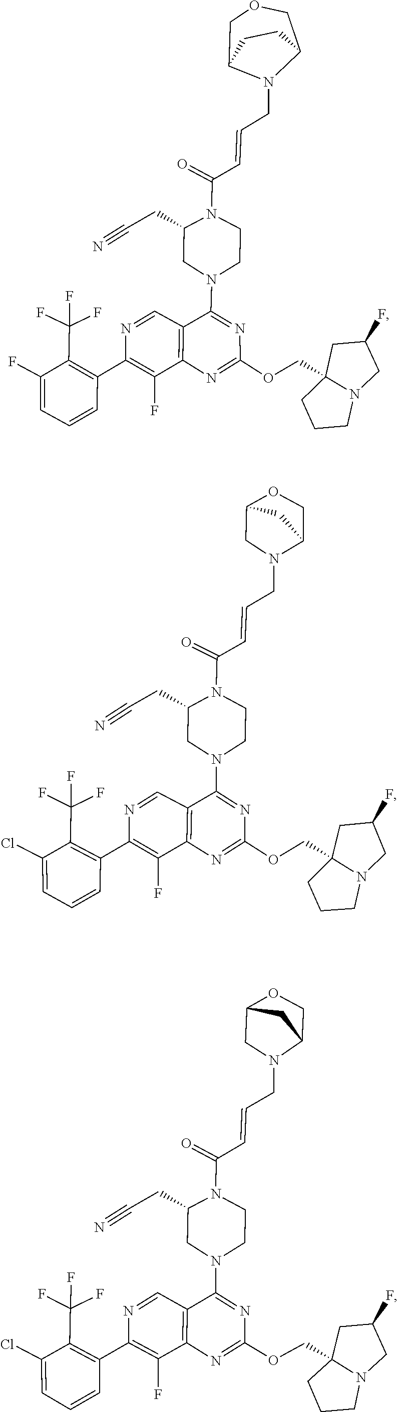 [SMILES: N#CC[C@H]1CN(c2nc(OC[C@@]34CCCN3C[C@H](F)C4)nc3c(F)c(-c4cccc(F)c4C(F)(F)F)ncc23)CCN1C(=O)/C=C/CN1[C@@H]2CC[C@H]1COC2 ; N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1C[C@@H]2C[C@H]1CO2 ; N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1C[C@H]2C[C@@H]1CO2]

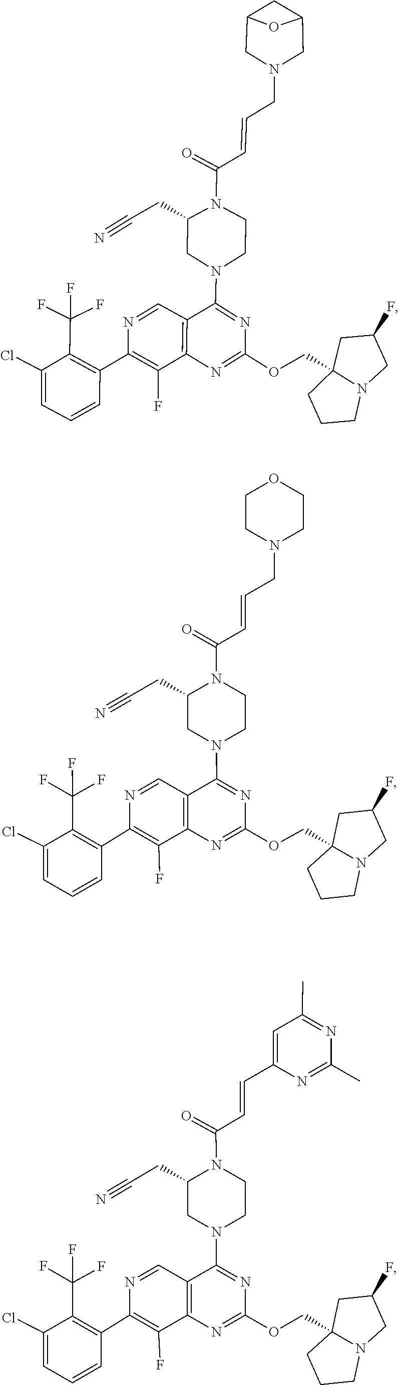 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CC2CC(C1)O2 ; N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CCOCC1 ; Cc1cc(/C=C/C(=O)N2CCN(c3nc(OC[C@@]45CCCN4C[C@H](F)C5)nc4c(F)c(-c5cccc(Cl)c5C(F)(F)F)ncc34)C[C@@H]2CC#N)nc(C)n1]

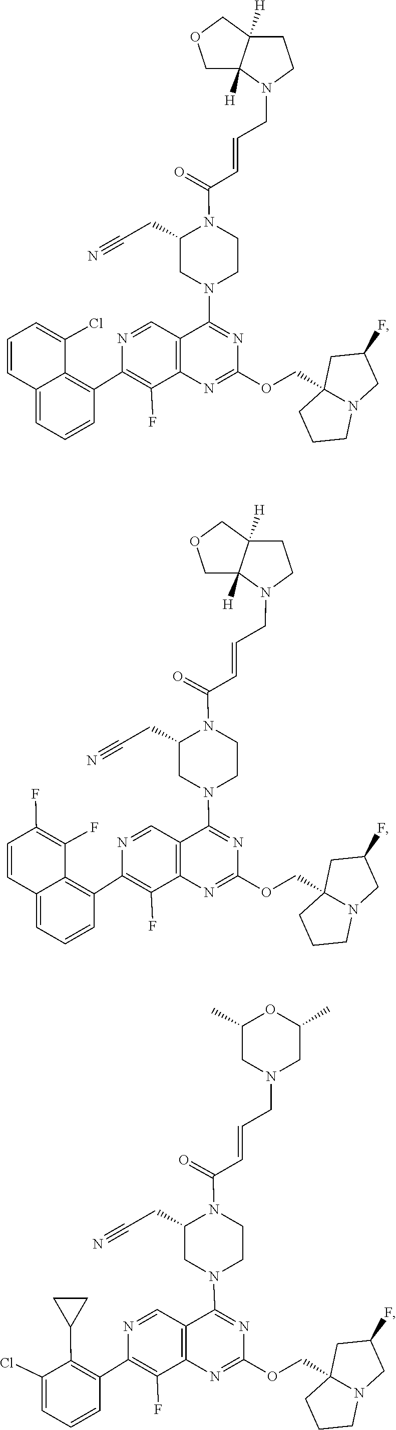 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@@H]2COC[C@H]21 ; N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CC[C@@H]2COC[C@H]21 ; C[C@@H]1CN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)C[C@H](C)O1]

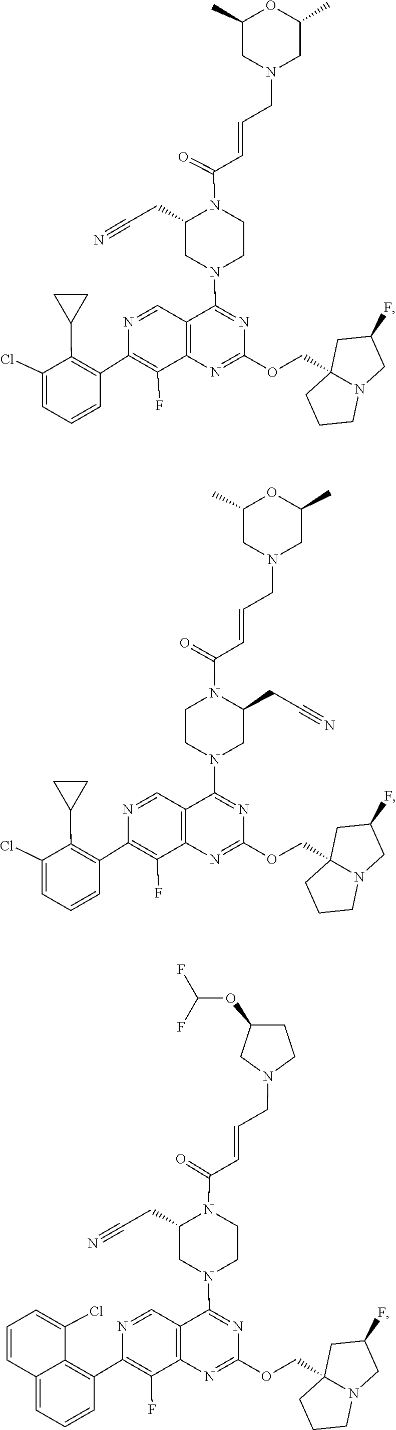 [SMILES: C[C@@H]1CN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)C[C@@H](C)O1 ; C[C@H]1CN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)C[C@H](C)O1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@H](OC(F)F)C1]

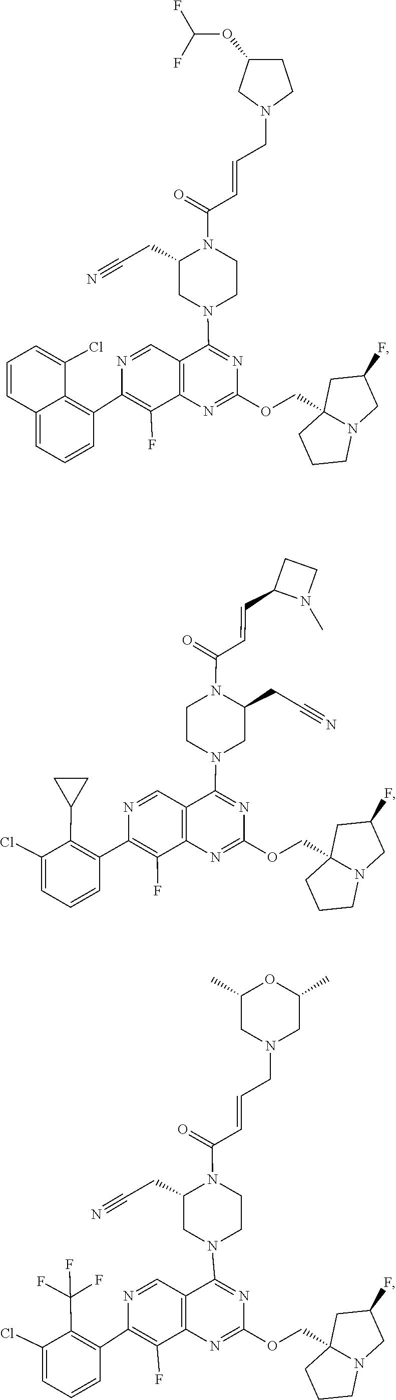 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@@H](OC(F)F)C1 ; CN1CC[C@@H]1/C=C/C(=O)N1CCN(c2nc(OC[C@@]34CCCN3C[C@H](F)C4)nc3c(F)c(-c4cccc(Cl)c4C4CC4)ncc23)C[C@@H]1CC#N ; C[C@@H]1CN(C/C=C/C(=O)N2CCN(c3nc(OC[C@@]45CCCN4C[C@H](F)C5)nc4c(F)c(-c5cccc(Cl)c5C(F)(F)F)ncc34)C[C@@H]2CC#N)C[C@H](C)O1]

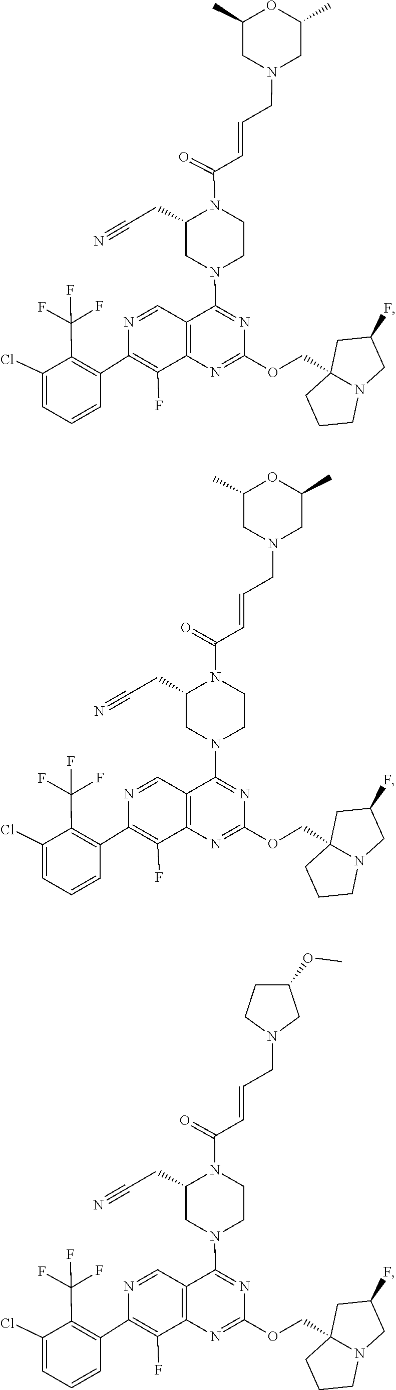 [SMILES: C[C@@H]1CN(C/C=C/C(=O)N2CCN(c3nc(OC[C@@]45CCCN4C[C@H](F)C5)nc4c(F)c(-c5cccc(Cl)c5C(F)(F)F)ncc34)C[C@@H]2CC#N)C[C@@H](C)O1 ; C[C@H]1CN(C/C=C/C(=O)N2CCN(c3nc(OC[C@@]45CCCN4C[C@H](F)C5)nc4c(F)c(-c5cccc(Cl)c5C(F)(F)F)ncc34)C[C@@H]2CC#N)C[C@H](C)O1 ; CO[C@H]1CCN(C/C=C/C(=O)N2CCN(c3nc(OC[C@@]45CCCN4C[C@H](F)C5)nc4c(F)c(-c5cccc(Cl)c5C(F)(F)F)ncc34)C[C@@H]2CC#N)C1]

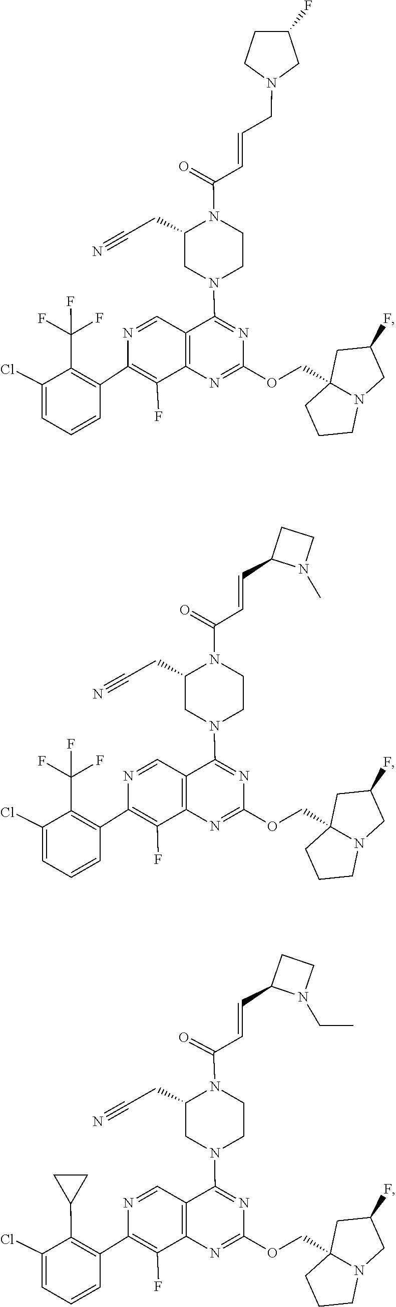 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CC[C@H](F)C1 ; CN1CC[C@@H]1/C=C/C(=O)N1CCN(c2nc(OC[C@@]34CCCN3C[C@H](F)C4)nc3c(F)c(-c4cccc(Cl)c4C(F)(F)F)ncc23)C[C@@H]1CC#N ; CCN1CC[C@@H]1/C=C/C(=O)N1CCN(c2nc(OC[C@@]34CCCN3C[C@H](F)C4)nc3c(F)c(-c4cccc(Cl)c4C4CC4)ncc23)C[C@@H]1CC#N]

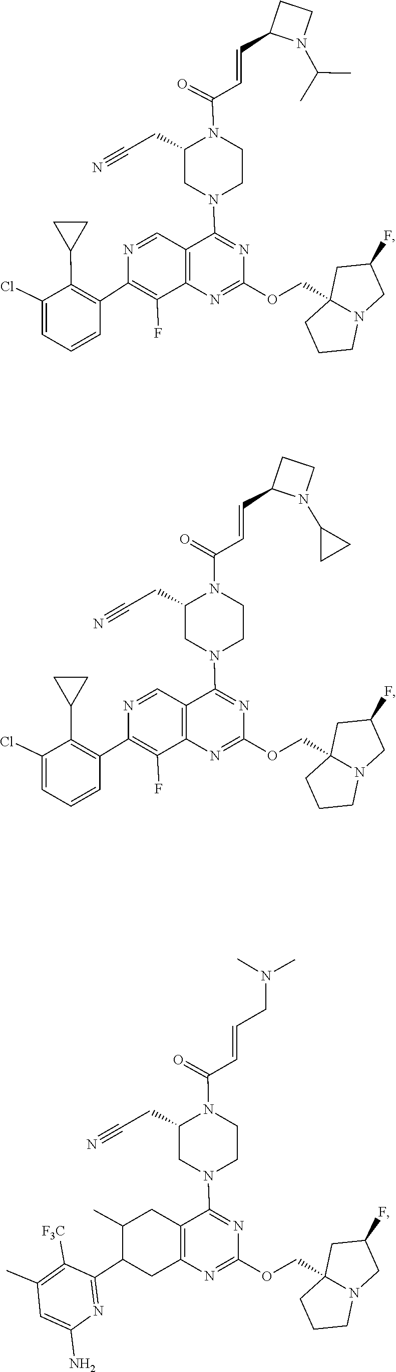 [SMILES: CC(C)N1CC[C@@H]1/C=C/C(=O)N1CCN(c2nc(OC[C@@]34CCCN3C[C@H](F)C4)nc3c(F)c(-c4cccc(Cl)c4C4CC4)ncc23)C[C@@H]1CC#N ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/[C@H]1CCN1C1CC1 ; Cc1cc(N)nc(C2Cc3nc(OC[C@@]45CCCN4C[C@H](F)C5)nc(N4CCN(C(=O)/C=C/CN(C)C)[C@@H](CC#N)C4)c3CC2C)c1C(F)(F)F]

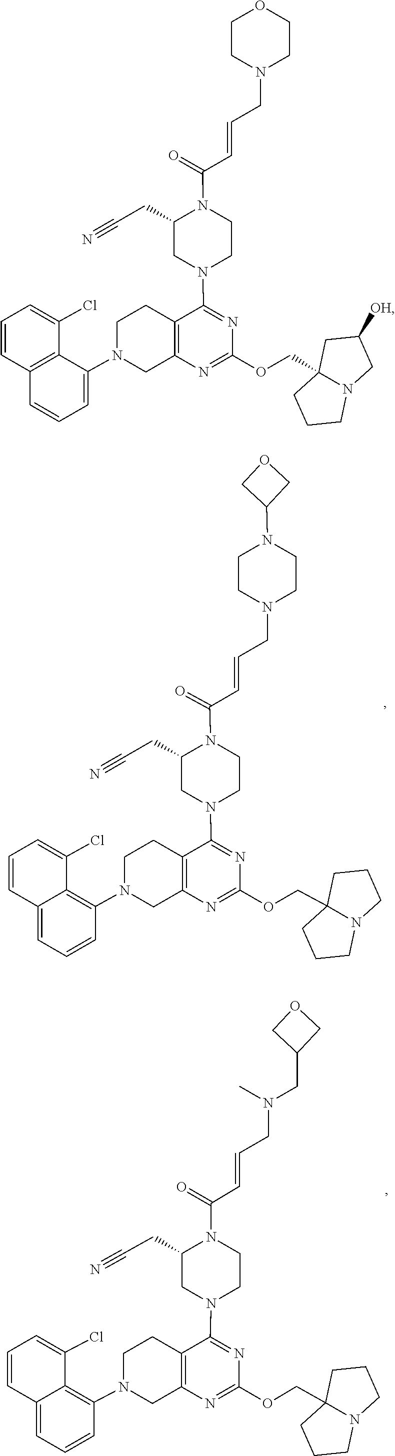 [SMILES: N#CC[C@H]1CN(c2nc(OC[C@@]34CCCN3C[C@H](O)C4)nc3c2CCN(c2cccc4cccc(Cl)c24)C3)CCN1C(=O)/C=C/CN1CCOCC1 ; N#CC[C@H]1CN(c2nc(OCC34CCCN3CCC4)nc3c2CCN(c2cccc4cccc(Cl)c24)C3)CCN1C(=O)/C=C/CN1CCN(C2COC2)CC1 ; CN(C/C=C/C(=O)N1CCN(c2nc(OCC34CCCN3CCC4)nc3c2CCN(c2cccc4cccc(Cl)c24)C3)C[C@@H]1CC#N)CC1COC1]

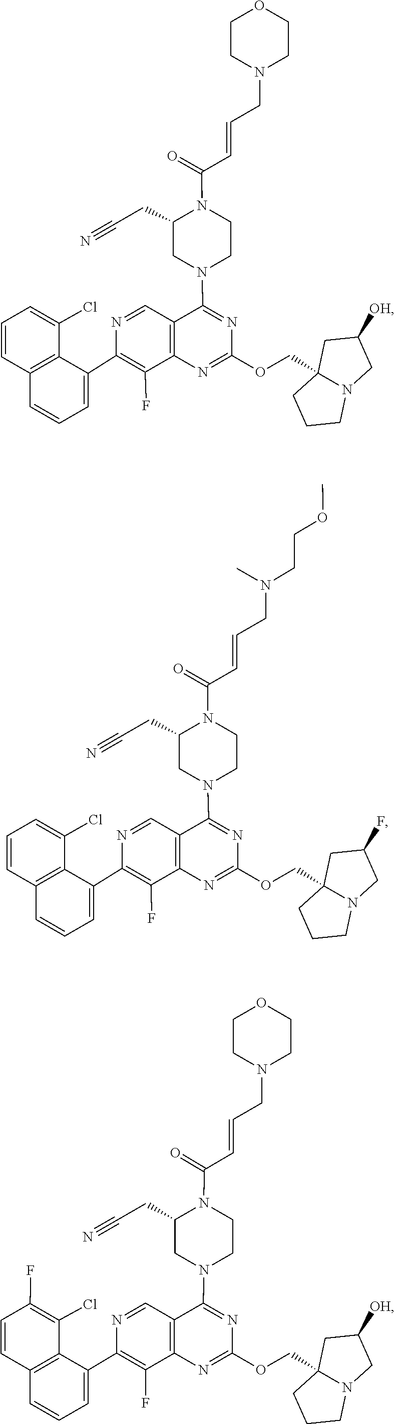 [SMILES: N#CC[C@H]1CN(c2nc(OC[C@@]34CCCN3C[C@H](O)C4)nc3c(F)c(-c4cccc5cccc(Cl)c45)ncc23)CCN1C(=O)/C=C/CN1CCOCC1 ; COCCN(C)C/C=C/C(=O)N1CCN(c2nc(OC[C@@]34CCCN3C[C@H](F)C4)nc3c(F)c(-c4cccc5cccc(Cl)c45)ncc23)C[C@@H]1CC#N ; N#CC[C@H]1CN(c2nc(OC[C@@]34CCCN3C[C@H](O)C4)nc3c(F)c(-c4cccc5ccc(F)c(Cl)c45)ncc23)CCN1C(=O)/C=C/CN1CCOCC1]

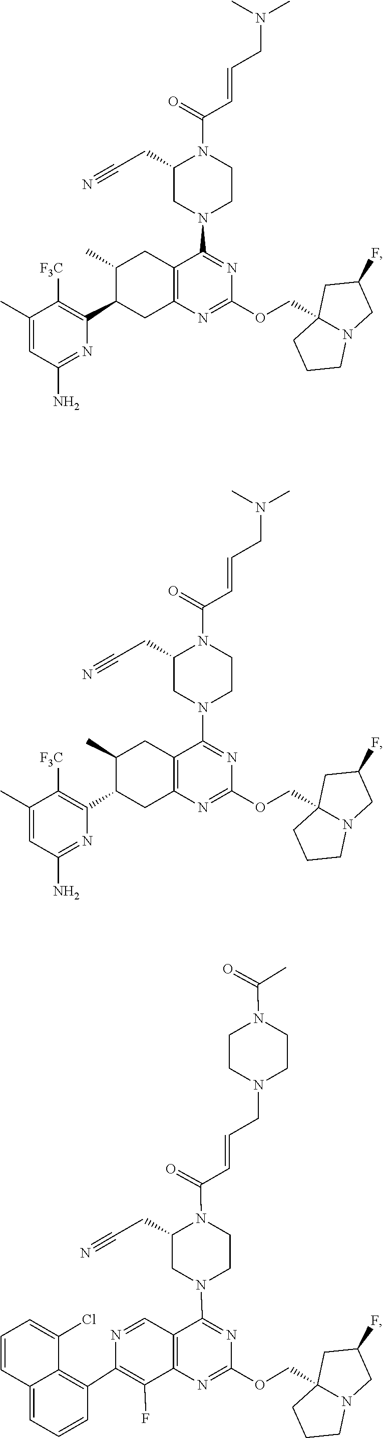

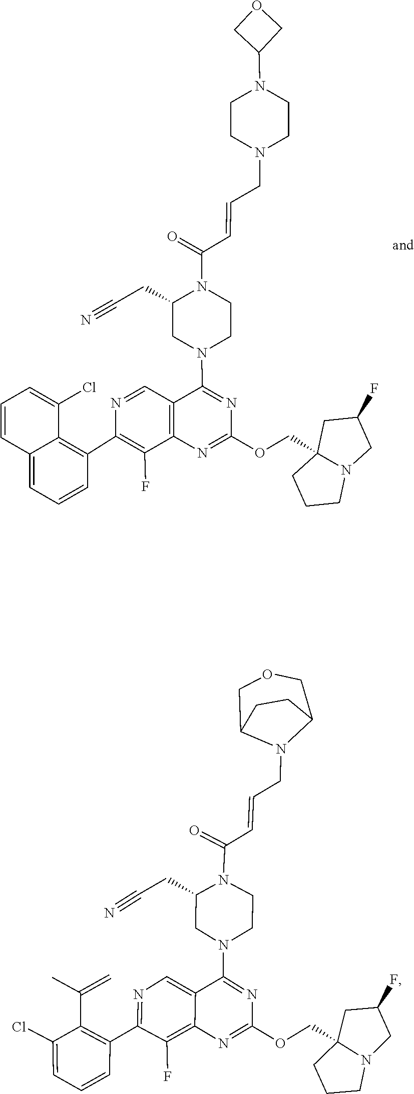 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCN(C2COC2)CC1 ; C=C(C)c1c(Cl)cccc1-c1ncc2c(N3CCN(C(=O)/C=C/CN4C5CCC4COC5)[C@@H](CC#N)C3)nc(OC[C@@]34CCCN3C[C@H](F)C4)nc2c1F]

or a salt thereof, and/or an isotopologue thereof.
Embodiment 318. The compound of any one of embodiments 1 to 316, wherein the compound is selected from the group consisting of:

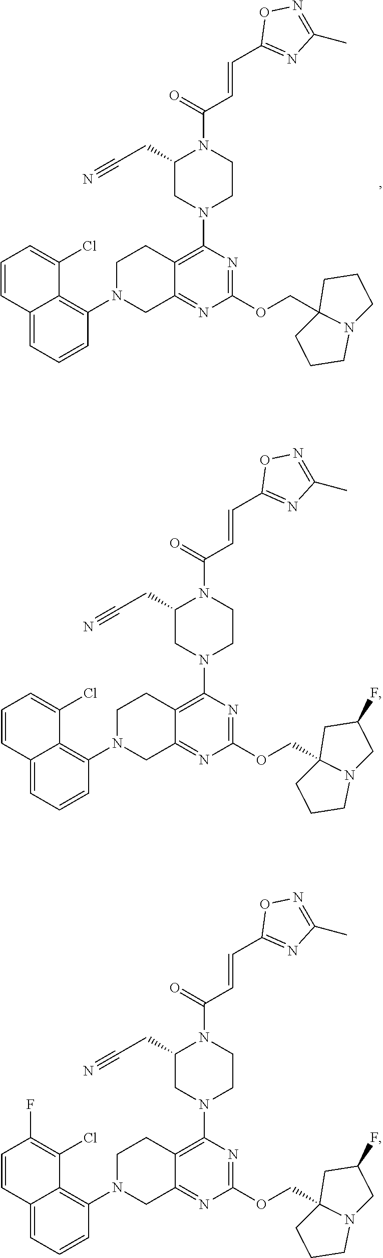 [SMILES: Cc1noc(/C=C/C(=O)N2CCN(c3nc(OCC45CCCN4CCC5)nc4c3CCN(c3cccc5cccc(Cl)c35)C4)C[C@@H]2CC#N)n1 ; Cc1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1 ; Cc1noc(/C=C/C(=O)N2CCN(c3nc(OC[C@@]45CCCN4C[C@H](F)C5)nc4c3CCN(c3cccc5ccc(F)c(Cl)c35)C4)C[C@@H]2CC#N)n1]

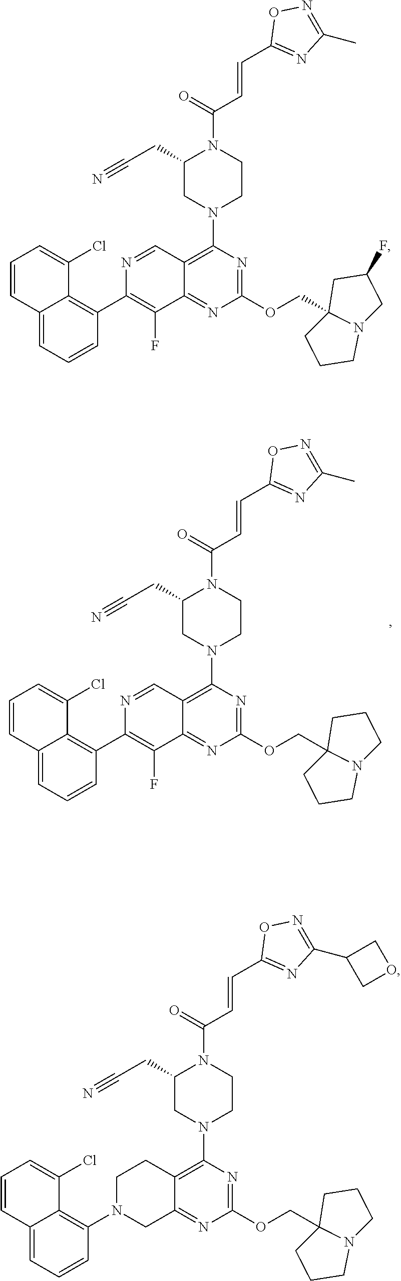 [SMILES: Cc1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; Cc1noc(/C=C/C(=O)N2CCN(c3nc(OCC45CCCN4CCC5)nc4c(F)c(-c5cccc6cccc(Cl)c56)ncc34)C[C@@H]2CC#N)n1 ; N#CC[C@H]1CN(c2nc(OCC34CCCN3CCC4)nc3c2CCN(c2cccc4cccc(Cl)c24)C3)CCN1C(=O)/C=C/c1nc(C2COC2)no1]

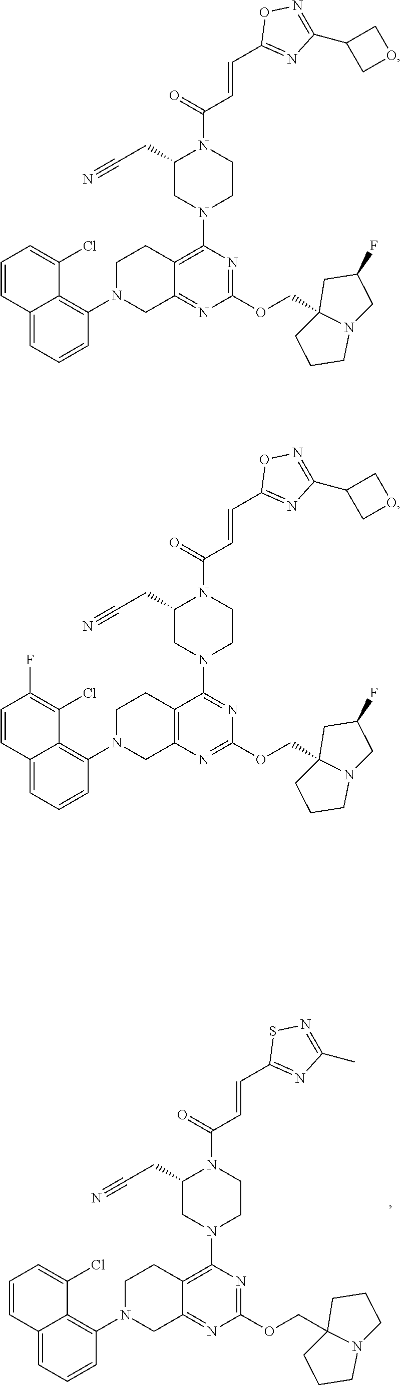 [SMILES: N#CC[C@H]1CN(c2nc(OC[C@@]34CCCN3C[C@H](F)C4)nc3c2CCN(c2cccc4cccc(Cl)c24)C3)CCN1C(=O)/C=C/c1nc(C2COC2)no1 ; N#CC[C@H]1CN(c2nc(OC[C@@]34CCCN3C[C@H](F)C4)nc3c2CCN(c2cccc4ccc(F)c(Cl)c24)C3)CCN1C(=O)/C=C/c1nc(C2COC2)no1 ; Cc1nsc(/C=C/C(=O)N2CCN(c3nc(OCC45CCCN4CCC5)nc4c3CCN(c3cccc5cccc(Cl)c35)C4)C[C@@H]2CC#N)n1]

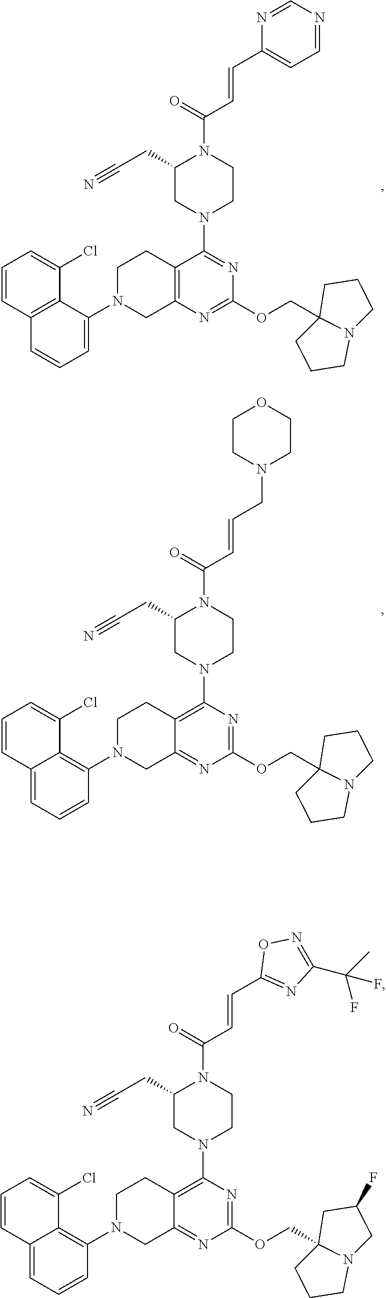 [SMILES: N#CC[C@H]1CN(c2nc(OCC34CCCN3CCC4)nc3c2CCN(c2cccc4cccc(Cl)c24)C3)CCN1C(=O)/C=C/c1ccncn1 ; N#CC[C@H]1CN(c2nc(OCC34CCCN3CCC4)nc3c2CCN(c2cccc4cccc(Cl)c24)C3)CCN1C(=O)/C=C/CN1CCOCC1 ; CC(F)(F)c1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1]

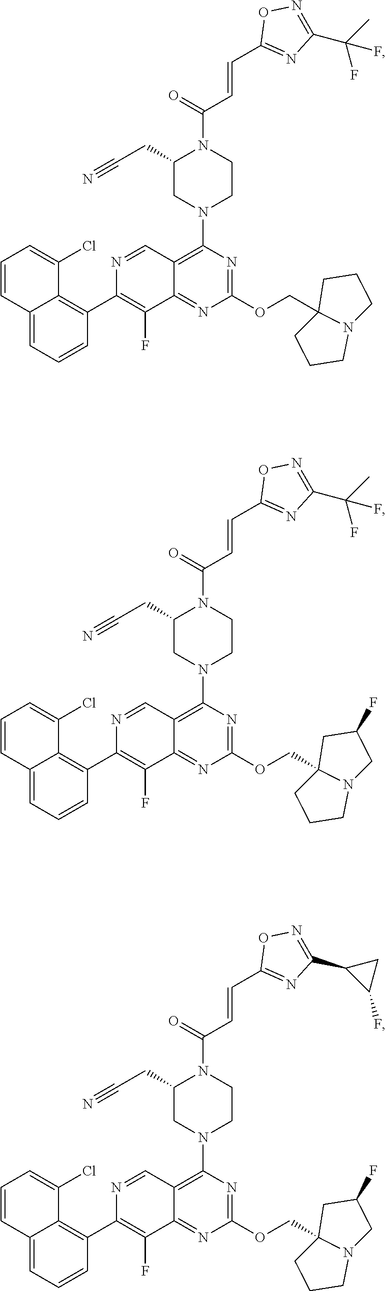 [SMILES: CC(F)(F)c1noc(/C=C/C(=O)N2CCN(c3nc(OCC45CCCN4CCC5)nc4c(F)c(-c5cccc6cccc(Cl)c56)ncc34)C[C@@H]2CC#N)n1 ; CC(F)(F)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/c1nc([C@H]2C[C@@H]2F)no1]

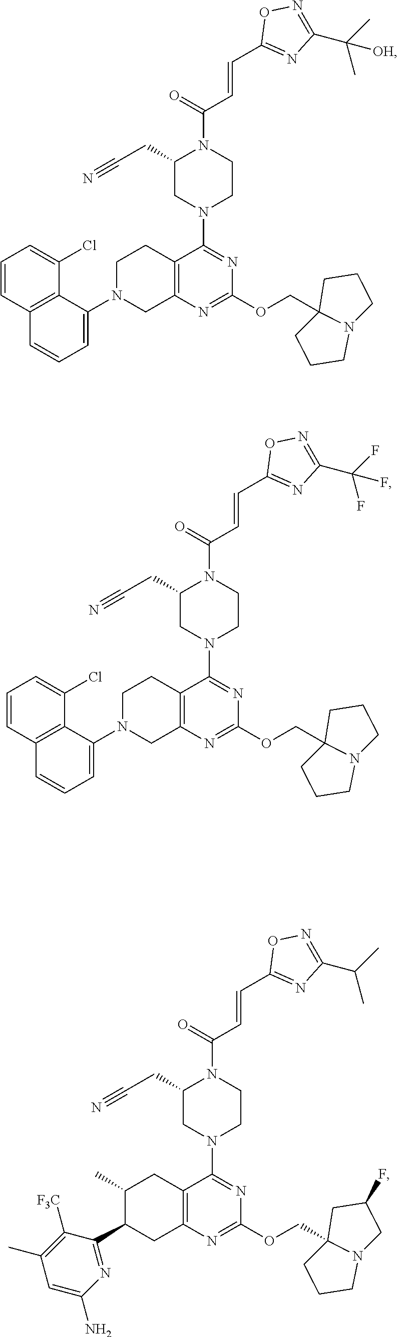 [SMILES: CC(C)(O)c1noc(/C=C/C(=O)N2CCN(c3nc(OCC45CCCN4CCC5)nc4c3CCN(c3cccc5cccc(Cl)c35)C4)C[C@@H]2CC#N)n1 ; N#CC[C@H]1CN(c2nc(OCC34CCCN3CCC4)nc3c2CCN(c2cccc4cccc(Cl)c24)C3)CCN1C(=O)/C=C/c1nc(C(F)(F)F)no1 ; Cc1cc(N)nc([C@@H]2Cc3nc(OC[C@@]45CCCN4C[C@H](F)C5)nc(N4CCN(C(=O)/C=C/c5nc(C(C)C)no5)[C@@H](CC#N)C4)c3C[C@H]2C)c1C(F)(F)F]

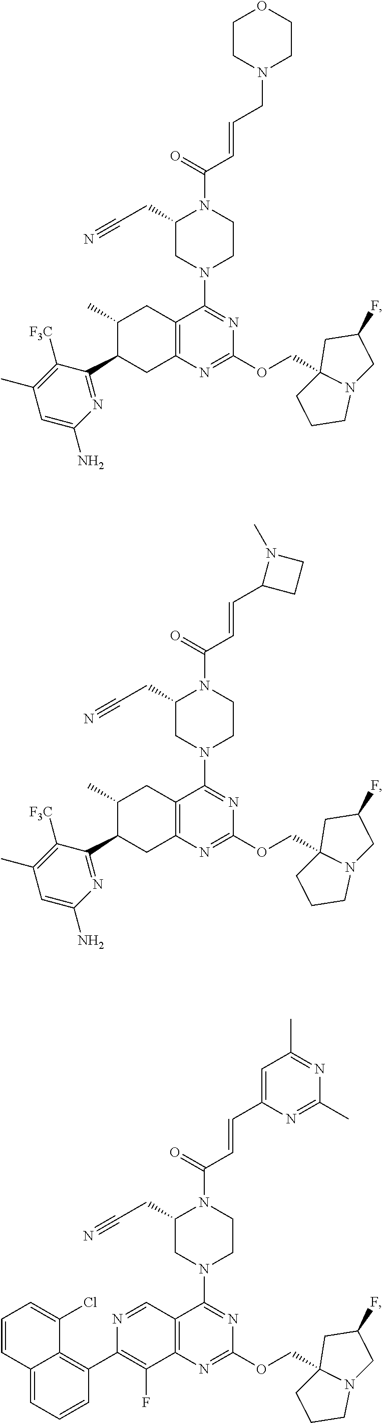 [SMILES: Cc1cc(N)nc([C@@H]2Cc3nc(OC[C@@]45CCCN4C[C@H](F)C5)nc(N4CCN(C(=O)/C=C/CN5CCOCC5)[C@@H](CC#N)C4)c3C[C@H]2C)c1C(F)(F)F ; Cc1cc(N)nc([C@@H]2Cc3nc(OC[C@@]45CCCN4C[C@H](F)C5)nc(N4CCN(C(=O)/C=C/C5CCN5C)[C@@H](CC#N)C4)c3C[C@H]2C)c1C(F)(F)F ; Cc1cc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)nc(C)n1]

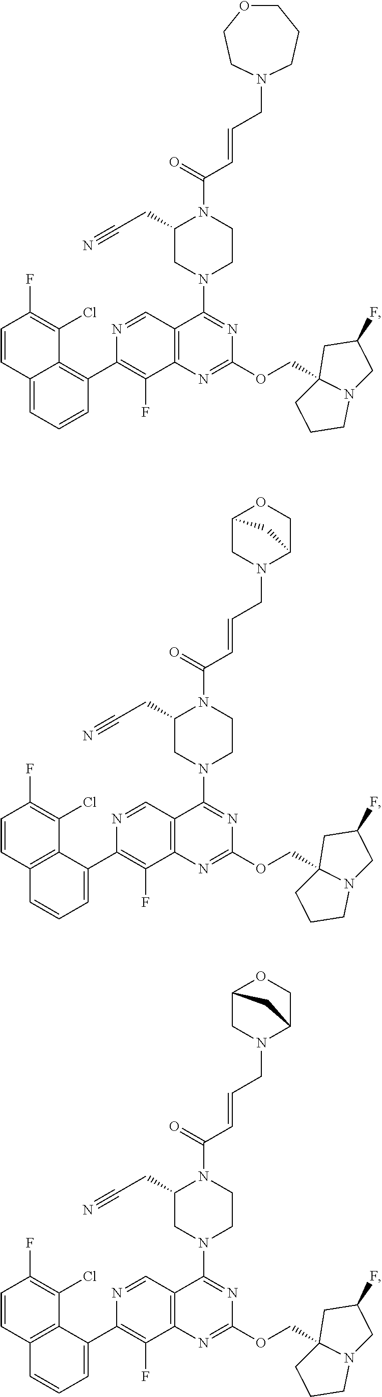 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCCOCC1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@@H]2C[C@H]1CO2 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@H]2C[C@@H]1CO2]

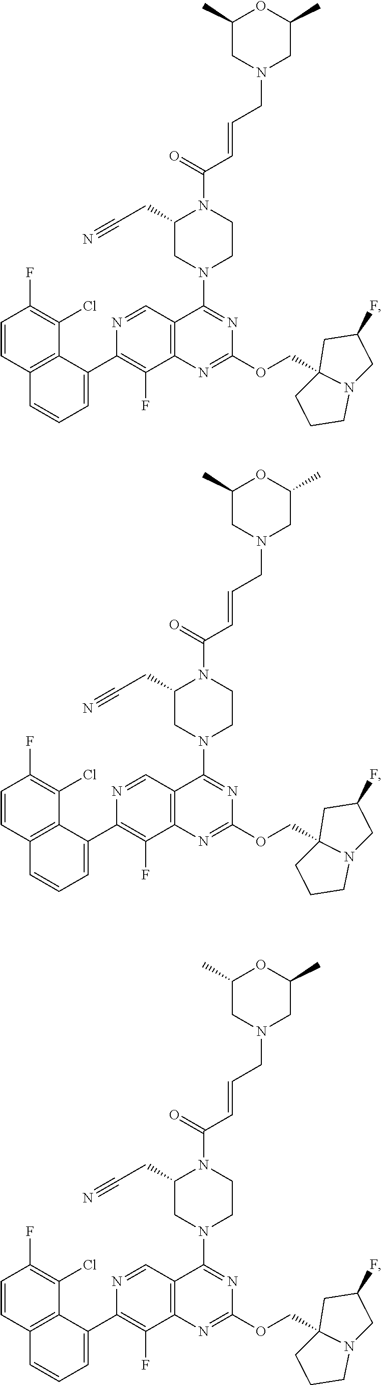 [SMILES: C[C@@H]1CN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C[C@H](C)O1 ; C[C@@H]1CN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C[C@@H](C)O1 ; C[C@H]1CN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C[C@H](C)O1]

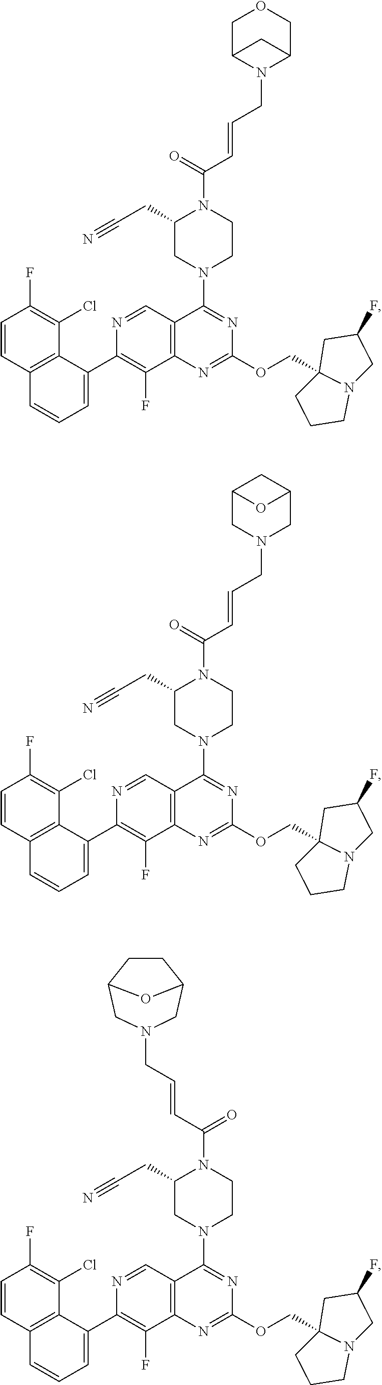 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C2COCC1C2 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2CC(C1)O2 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2CCC(C1)O2]

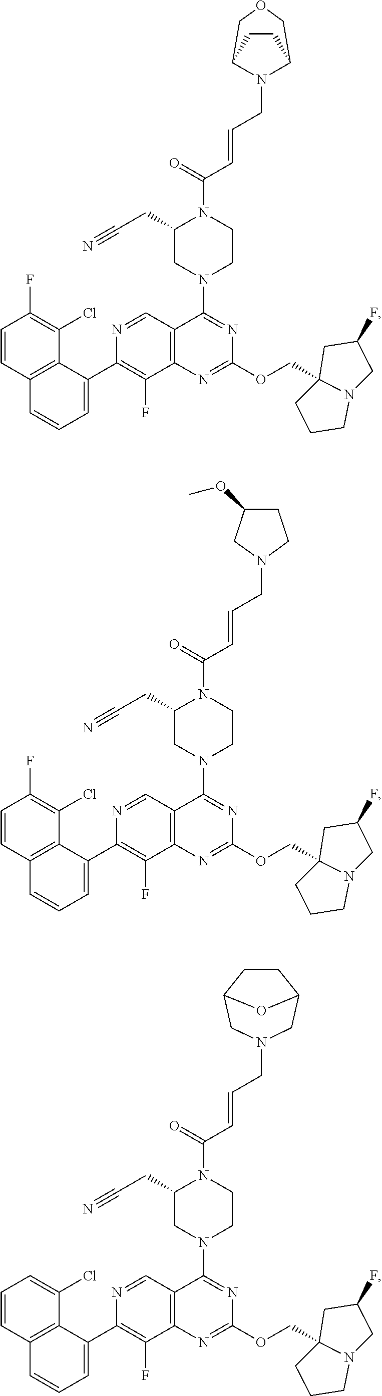 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1[C@@H]2CC[C@H]1COC2 ; CO[C@H]1CCN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CCC(C1)O2]

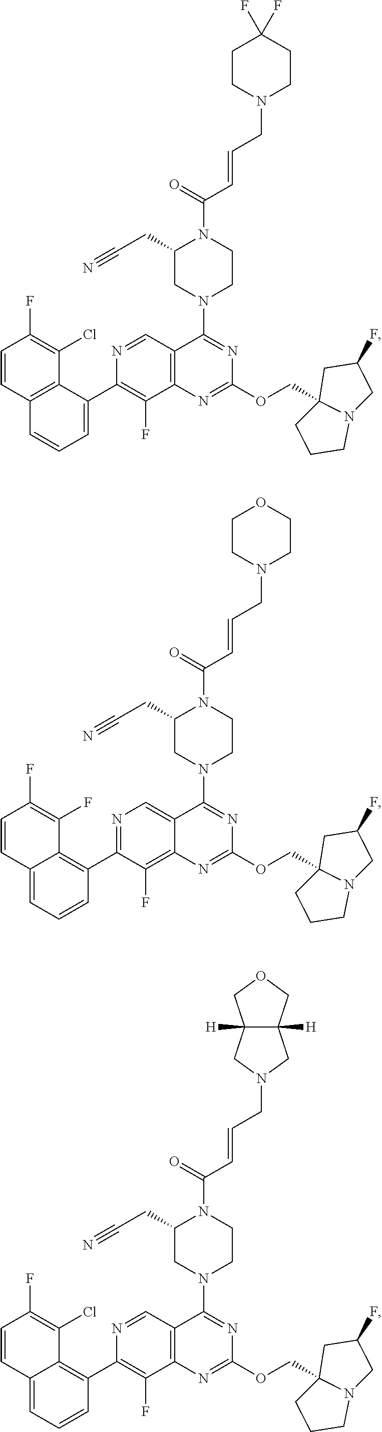 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCC(F)(F)CC1 ; N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCOCC1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@H]2COC[C@H]2C1]

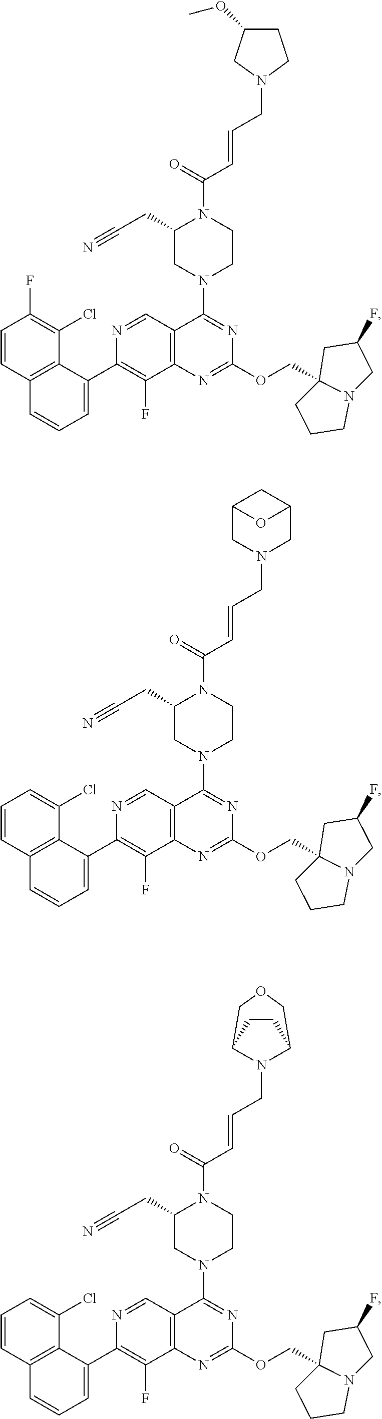 [SMILES: CO[C@@H]1CCN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CC(C1)O2 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1[C@@H]2CC[C@H]1COC2]

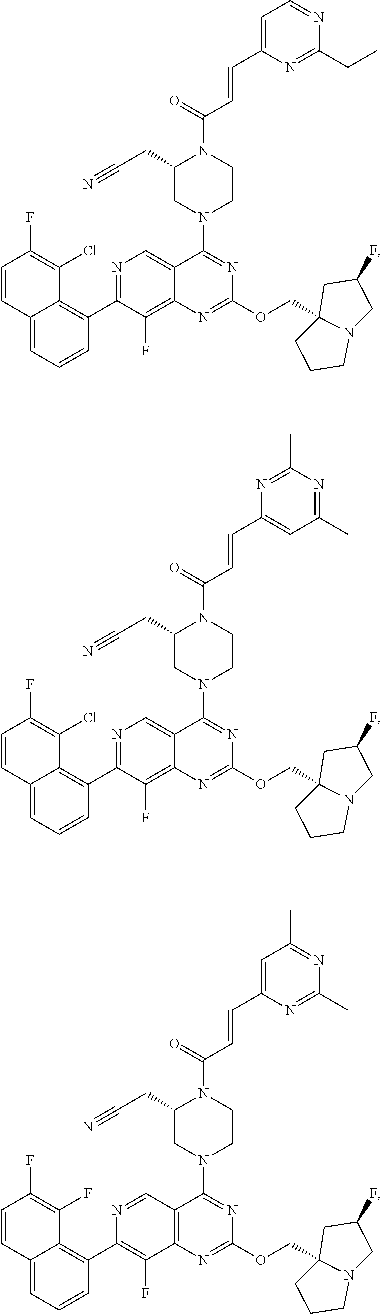 [SMILES: CCc1nccc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1 ; Cc1cc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)nc(C)n1 ; Cc1cc(/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)nc(C)n1]

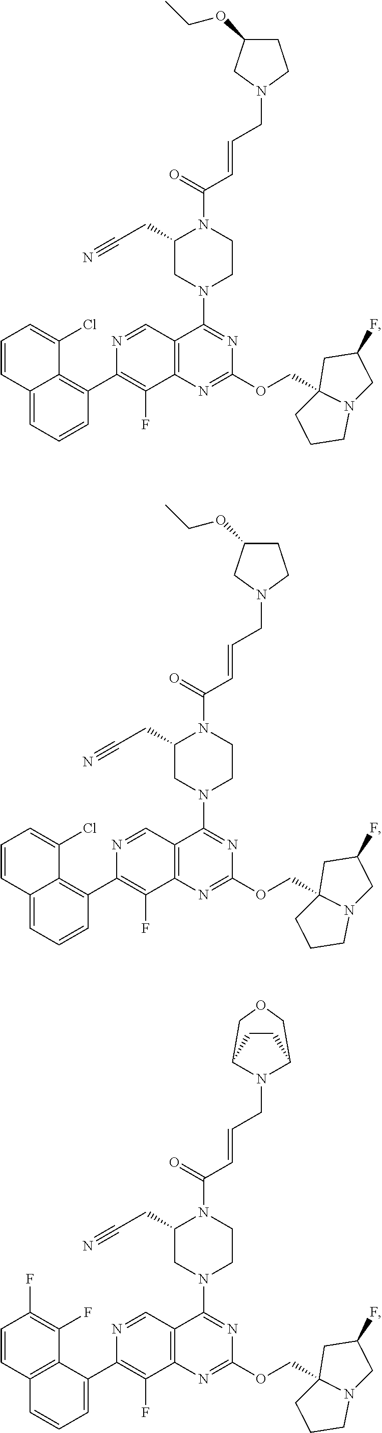 [SMILES: CCO[C@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1 ; CCO[C@@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1 ; N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1[C@@H]2CC[C@H]1COC2]

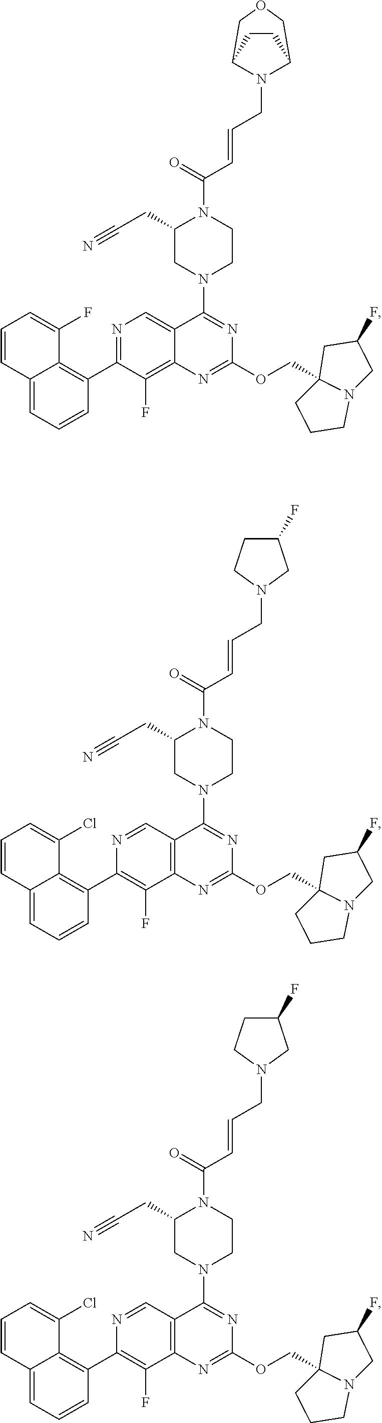 [SMILES: N#CC[C@H]1CN(c2nc(OC[C@@]34CCCN3C[C@H](F)C4)nc3c(F)c(-c4cccc5cccc(F)c45)ncc23)CCN1C(=O)/C=C/CN1[C@@H]2CC[C@H]1COC2 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@H](F)C1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@@H](F)C1]

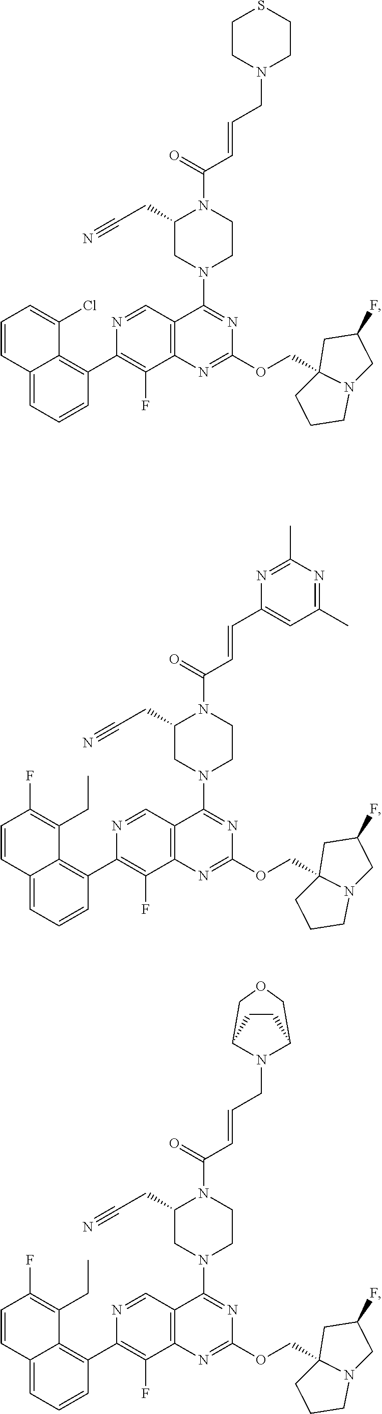 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCSCC1 ; CCc1c(F)ccc2cccc(-c3ncc4c(N5CCN(C(=O)/C=C/c6cc(C)nc(C)n6)[C@@H](CC#N)C5)nc(OC[C@@]56CCCN5C[C@H](F)C6)nc4c3F)c12 ; CCc1c(F)ccc2cccc(-c3ncc4c(N5CCN(C(=O)/C=C/CN6[C@@H]7CC[C@H]6COC7)[C@@H](CC#N)C5)nc(OC[C@@]56CCCN5C[C@H](F)C6)nc4c3F)c12]

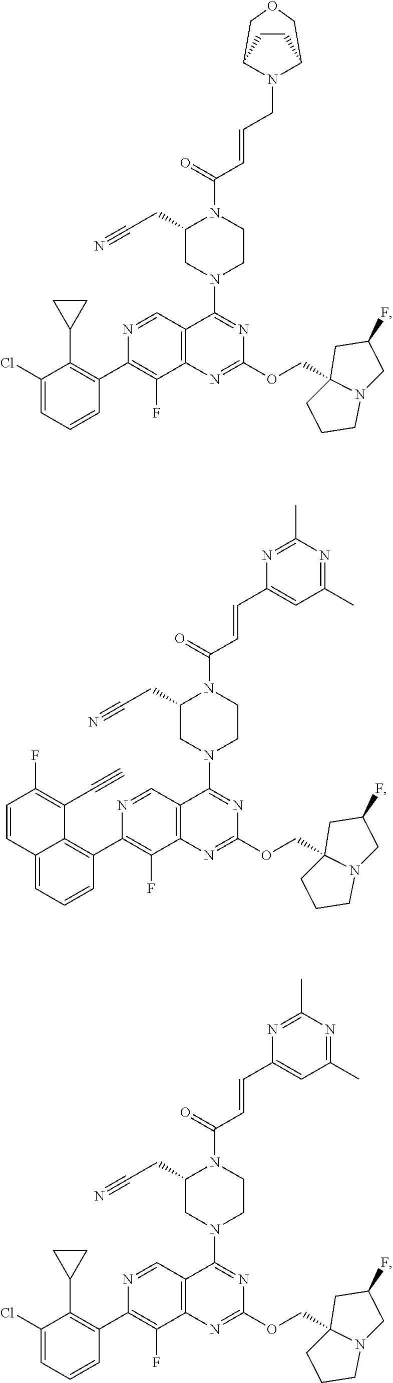 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1[C@@H]2CC[C@H]1COC2 ; C#Cc1c(F)ccc2cccc(-c3ncc4c(N5CCN(C(=O)/C=C/c6cc(C)nc(C)n6)[C@@H](CC#N)C5)nc(OC[C@@]56CCCN5C[C@H](F)C6)nc4c3F)c12 ; Cc1cc(/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)nc(C)n1]

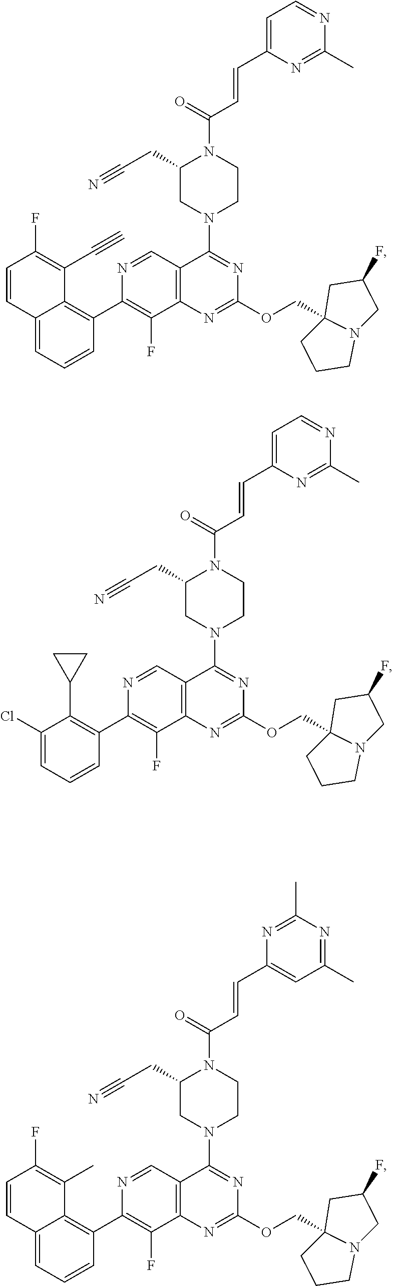 [SMILES: C#Cc1c(F)ccc2cccc(-c3ncc4c(N5CCN(C(=O)/C=C/c6ccnc(C)n6)[C@@H](CC#N)C5)nc(OC[C@@]56CCCN5C[C@H](F)C6)nc4c3F)c12 ; Cc1nccc(/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)n1 ; Cc1cc(/C=C/C(=O)N2CCN(c3nc(OC[C@@]45CCCN4C[C@H](F)C5)nc4c(F)c(-c5cccc6ccc(F)c(C)c56)ncc34)C[C@@H]2CC#N)nc(C)n1]

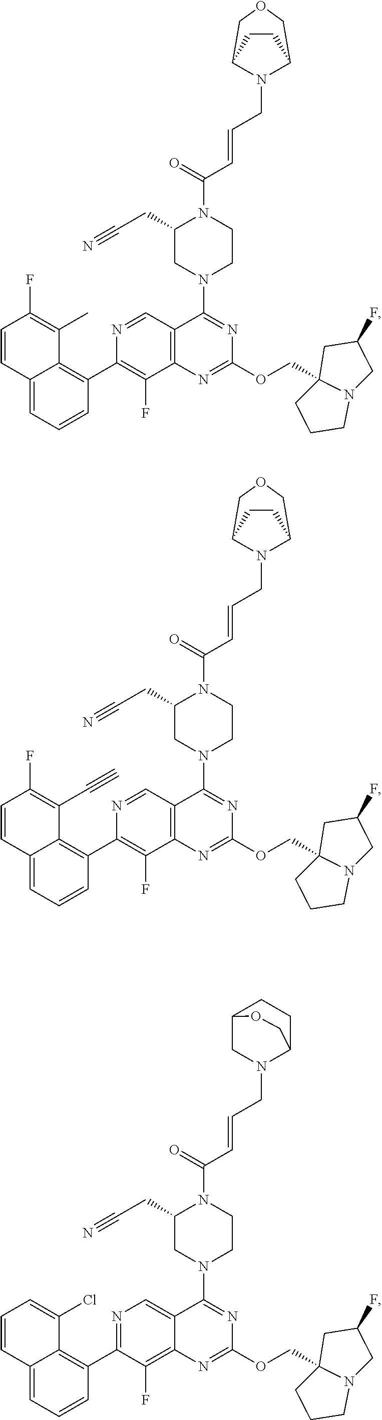 [SMILES: Cc1c(F)ccc2cccc(-c3ncc4c(N5CCN(C(=O)/C=C/CN6[C@@H]7CC[C@H]6COC7)[C@@H](CC#N)C5)nc(OC[C@@]56CCCN5C[C@H](F)C6)nc4c3F)c12 ; C#Cc1c(F)ccc2cccc(-c3ncc4c(N5CCN(C(=O)/C=C/CN6[C@@H]7CC[C@H]6COC7)[C@@H](CC#N)C5)nc(OC[C@@]56CCCN5C[C@H](F)C6)nc4c3F)c12 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CCC1CO2]

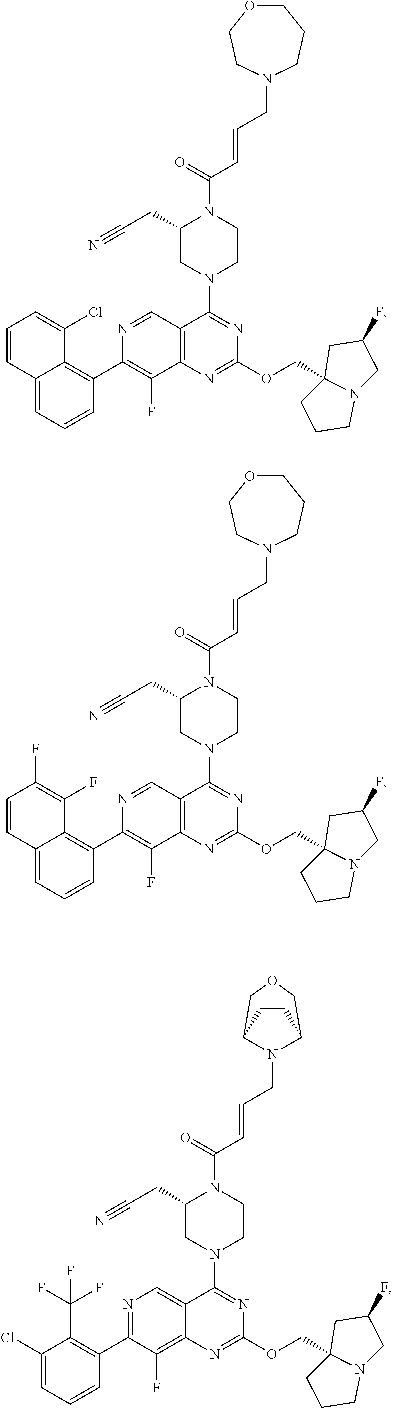 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCCOCC1 ; N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCCOCC1 ; N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1[C@@H]2CC[C@H]1COC2]

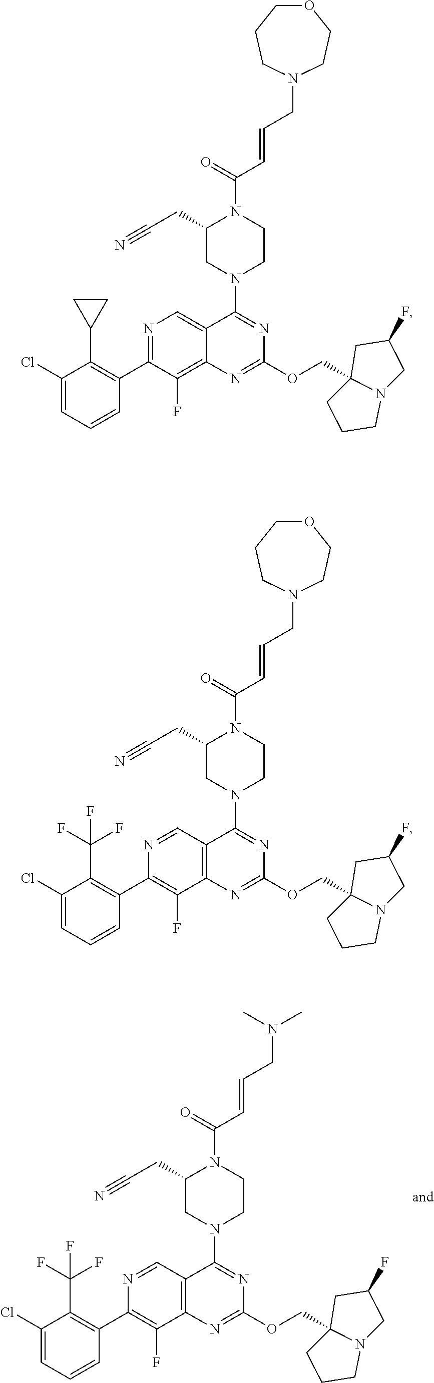 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CCCOCC1 ; N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CCCOCC1 ; CN(C)C/C=C/C(=O)N1CCN(c2nc(OC[C@@]34CCCN3C[C@H](F)C4)nc3c(F)c(-c4cccc(Cl)c4C(F)(F)F)ncc23)C[C@@H]1CC#N]

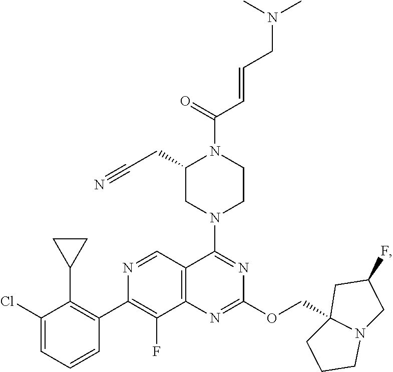 [SMILES: CN(C)C/C=C/C(=O)N1CCN(c2nc(OC[C@@]34CCCN3C[C@H](F)C4)nc3c(F)c(-c4cccc(Cl)c4C4CC4)ncc23)C[C@@H]1CC#N]

or a salt thereof, and/or an isotopologue thereof.
Embodiment 319. The compound of any one of embodiments 1 to 318, wherein the compound is not a salt.
Embodiment 320. The compound of any one of embodiments 1 to 318, wherein the compound is a salt.
Embodiment 321. The compound of embodiment 320, wherein the salt is a formate salt.
Embodiment 322. The compound of embodiment 320, wherein the salt is a trifluoroacetate salt.
Embodiment 323. The compound of embodiment 320, wherein the salt is a pharmaceutically acceptable salt.
Embodiment 324. A pharmaceutical formulation comprising the compound of any one of embodiments 1 to 322, wherein when the compound is a salt, the salt is a pharmaceutically acceptable salt, and a pharmaceutically acceptable carrier.
Embodiment 325. A method of treating or suppressing cancer comprising: administering a therapeutically effective amount of a compound of any one of embodiments 1 to 323, wherein when the compound is a salt, the salt is a pharmaceutically acceptable salt, or a pharmaceutical formulation according to embodiment 324, to a subject in need thereof.
Embodiment 326. The method of embodiment 325, wherein the cancer is selected from the group consisting of: lung, colorectal, pancreatic, bile duct, thyroid, gall bladder, uterine, mesothelioma, cervical, and bladder cancers.
Embodiment 327. The method of embodiment 326, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cellmyeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cellmalignancies, pediatric neuroblastoma, and melanoma.
Embodiment 328. The method of any one of embodiments 325 to 327, wherein the cancer is a KRAS G12C mediated cancer.
Embodiment 329. The method of any one of embodiments 325 to 327, wherein the subject has been diagnosed as having a KRAS G12C mediated cancer.
Embodiment 330. The method of any one of embodiments 325 to 329, wherein the method further comprises administering to the subject a therapeutically effective amount of an additional chemotherapeutic agent.
Embodiment 331. A compound of any one of embodiments 1 to 323 or a pharmaceutical formulation according to embodiment 324 for use as a medicament.
Embodiment 332. A compound of any one of embodiments 1 to 323 or a pharmaceutical formulation according to embodiment 324, for use in treating or suppressing cancer, wherein when the compound is a salt, the salt is a pharmaceutically acceptable salt.
Embodiment 333. The compound or pharmaceutical composition for use of embodiment 332, wherein the cancer is selected from the group consisting of: lung, colorectal, pancreatic, bile duct, thyroid, gall bladder, uterine, mesothelioma, cervical, and bladder cancers.
Embodiment 334. The compound or pharmaceutical composition for use of embodiment 332, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.
Embodiment 335. The compound or pharmaceutical composition for use of any one of embodiments 332-334, wherein the cancer is a KRAS G12C mediated cancer.
Embodiment 336. The compound or pharmaceutical composition for use of any one of embodiments 332-334, wherein the subject has been diagnosed as having a KRAS G12C mediated cancer.
Embodiment 337. The compound or pharmaceutical composition for use of any one of embodiments 332-336, wherein the compound or pharmaceutical composition is configured for administration with a therapeutically effective amount of an additional chemotherapeutic agent.
Embodiment 338. The compound or pharmaceutical composition for use of any one of embodiments 332-337, wherein the compound or pharmaceutical composition is configured for administration in a therapeutically effective amount.
Embodiment 339. A compound of any one of embodiments 1 to 323 or a pharmaceutical formulation according to embodiment 324 for use in the manufacturing of a medicament for treating or suppressing cancer, wherein when the compound is a salt, the salt is a pharmaceutically acceptable salt.
Embodiment 340. The compound or pharmaceutical composition for use of embodiment 339, wherein the cancer is selected from the group consisting of: lung, colorectal, pancreatic, bile duct, thyroid, gall bladder, uterine, mesothelioma, cervical, and bladder cancers.
Embodiment 341. The compound or pharmaceutical composition for use of embodiment 339, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.
Embodiment 342. The compound or pharmaceutical composition for use of any one of embodiments 339-341, wherein the cancer is a KRAS G12C mediated cancer.
Embodiment 343. The compound or pharmaceutical composition for use of any one of embodiments 339-341, wherein the subject has been diagnosed as having a KRAS G12C mediated cancer.
Embodiment 344. The compound or pharmaceutical composition for use of any one of embodiments 339-343, wherein the compound or pharmaceutical composition is configured for administration with a therapeutically effective amount of an additional chemotherapeutic agent.
Embodiment 345 The compound or pharmaceutical composition for use of any one of embodiments 339-344, wherein the medicament comprises a therapeutically effective amount of the compound or composition.
Embodiment 346. Use of a compound of any one of embodiments 1 to 323 or a pharmaceutical formulation according to embodiment 324 in the manufacturing of a medicament for treating or suppressing cancer, wherein when the compound is a salt, the salt is a pharmaceutically acceptable salt.
Embodiment 347. The use of embodiment 346, wherein the cancer is selected from the group consisting of: lung, colorectal, pancreatic, bile duct, thyroid, gall bladder, uterine, mesothelioma, cervical, and bladder cancers.
Embodiment 348. The use of embodiment 346, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.
Embodiment 349. The use of any one of embodiments 346-348, wherein the cancer is a KRAS G12C mediated cancer.
Embodiment 350. The use of any one of embodiments 346-348, wherein the subject has been diagnosed as having a KRAS G12C mediated cancer.
Embodiment 351. The use of any one of embodiments 346-350, wherein the compound or pharmaceutical composition is configured for administration with a therapeutically effective amount of an additional chemotherapeutic agent.
Embodiment 352. The use of any one of embodiments 346-351, wherein the medicament comprises a therapeutically effective amount of the compound or pharmaceutical composition.
Embodiment 353. Use of a compound of any one of embodiments 1 to 323 or a pharmaceutical formulation according to embodiment 324 for treating or suppressing cancer, wherein when the compound is a salt, the salt is a pharmaceutically acceptable salt.
Embodiment 354. The use of embodiment 353, wherein the cancer is selected from the group consisting of: lung, colorectal, pancreatic, bile duct, thyroid, gall bladder, uterine, mesothelioma, cervical, and bladder cancers.
Embodiment 353. The use of embodiment 353, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.
Embodiment 356. The use of any one of embodiments 353-355, wherein the cancer is a KRAS G12C mediated cancer.
Embodiment 357. The use of any one of embodiments 353-355, wherein the subject has been diagnosed as having a KRAS G12C mediated cancer.
Embodiment 358. The use of any one of embodiments 353-357, wherein the compound or pharmaceutical composition is configured for administration with a therapeutically effective amount of an additional chemotherapeutic agent.
Embodiment 359. The use of any one of embodiments 353-358, wherein use involves a therapeutically effective amount of the compound or composition.

### General Synthetic Methods

With the exception of compounds 56, 57 and 58 in Table 1, the compounds of the instant disclosure were prepared according to methods described in the Examples section or variations thereof that would be within the knowledge of one of skill in the art. Compounds 56, 57 and 58, marked in Table 1 with “*” can be prepared using methods adapted from those described in the Examples section, variations thereof, or synthetic methods known to persons of skill in the art.

The starting materials and reagents used in preparing these compounds are either available from commercial suppliers such as MilliporeSigma., Bachem., etc. or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

### EXAMPLES

The following preparations of compounds of Formula (A), Formula (B) and Formula (C) and pharmaceutically acceptable salts thereof are given to enable those skilled in the art to more clearly understand and to practice the present disclosure. They should not be considered as limiting the scope of the disclosure, but merely as being illustrative and representative thereof.

The following abbreviations are used in this section:

| | |
|---|---|
| Boc | tert-butyloxycarbonyl |
| PMB | p-methoxybenzyl |
| ACN | acetonitrile |
| TFA | trifluoroacetic acid |
| FA | formic acid |
| mmol | millimoles |
| g | grams |
| mg | milligrams |
| h | hours |
| mL | milliliters |
| NMR | Nuclear magnetic resonance |
| MHz | megahertz |
| LCMS | Liquid chromatography-mass |
| | spectrometry |
| Rt | Retention time |
| m/z | Mass/charge |
| min | minutes |
| um | micrometers |
| mm | millimeters |
| umol | micromoles |

All reagents were obtained from commercial suppliers and used without further purification unless otherwise stated.

### SYNTHETIC EXAMPLES

### General Schemes

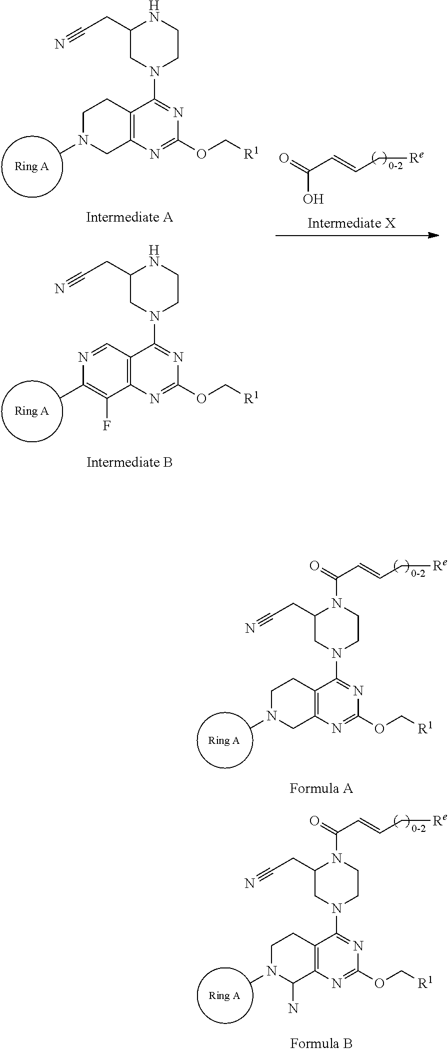

### General Scheme 1 (Method 1, Step 4) General Procedure for the Synthesis of Compounds of Formula (A) and Formula (B)

To a solution of NH piperazine intermediate A or B (1 eq) in dichloromethane was added substituted acrylic acid Intermediate X (2 eq), N,N-diisopropylethylamine (3 eq) and 2,4,6-tripropyl-1,3,5,2,4,6-trioxatriphosphinane 2,4,6-trioxide (1.5 eq, 50% purity in ethyl acetate) at 0° C. and the mixture was stirred at 25° C. for 1 h to provide crude product of Formula (A) or Formula (B).

### Intermediates

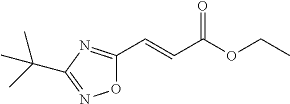 [SMILES: CCOC(=O)/C=C/c1nc(C(C)(C)C)no1]

### Example A (Method 4): (E)-ethyl 3-(3-(tert-butyl)-1,2,4-oxadiazol-5-yl)acrylate

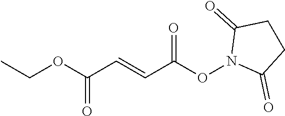 [SMILES: CCOC(=O)/C=C/C(=O)ON1C(=O)CCC1=O]

### Step 1: 2,5-dioxopyrrolidin-1-yl ethyl fumarate

To a solution of (E)-4-ethoxy-4-oxobut-2-enoic acid (30 g, 208.15 mmol) and 1-hydroxypyrrolidine-2,5-dione (71.87 g, 624.46 mmol) in acetonitrile (150 mL) was added 1-(3-Dimethylaminopropyl)-3-ethylcarbodiimide (79.81 g, 416.31 mmol), the mixture was stirred at 25° C. for 2 h. The mixture was diluted with water (150 mL) and extracted with ethyl acetate (3×100 mL). The combined organic layers were dried over sodium sulphate and concentrated in vacuo. The crude residue was diluted with ethyl acetate (50 mL) and the resulting precipitate was filtered affording 2,5-dioxopyrrolidin-1-yl ethyl fumarate (45 g, 86.05%) as a yellow oil: ¹H NMR (400 MHz, Chloroform -d) δ 7.15-6.92 (m, 2H), 4.29 (q, J=7.1 Hz, 2H), 2.86 (s, 4H), 1.32 (t, J=7.2 Hz, 3H). LCMS Rt=0.459 min, m/z=241.1 [M+H]⁺.

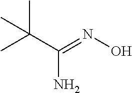

### Step 2: (Z)—N′-hydroxypivalimidamide

To a solution of 2,2-dimethylpropanenitrile (10 g, 120.29 mmol) in ethanol (100 mL) was added hydroxylamine;hydrochloride (12.54 g, 180.44 mmol) and potassium carbonate (33.25 g, 240.58 mmol). The mixture was stirred at 80° C. for 2 h. The mixture was concentrated to dryness in vacuo. The residue was diluted with water (100 mL) and extracted with ethyl acetate (3×300 mL). The combined organic layers were dried over sodium sulfate and concentrated in vacuo. The resulting residue was purified by column chromatography (silica gel, 100-200 mesh, 0-100% ethyl acetate in petroleum ether) affording (Z)—N′-hydroxypivalimidamide (3.54 g, 25%) as a blue solid: ¹H NMR (400 MHz, Dimethylsulfoxide-d6) δ 8.91-8.76 (m, 1H), 5.27-5.13 (m, 2H), 1.08 (s, 9H).

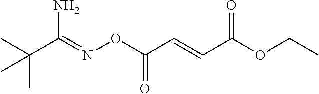 [SMILES: CCOC(=O)/C=C/C(=O)O/N=C(\N)C(C)(C)C]

### Step 3: (E)-ethyl 4-(((Z)-(1-amino-2,2-dimethylpropylidene)amino)oxy)-4-oxobut-2-enoate

To a solution of 2,5-dioxopyrrolidin-1-yl ethyl fumarate (2 g, 8.29 mmol) in dioxane (30 mL) were added potassium carbonate (3.44 g, 24.88 mmol) and (Z)—N′-hydroxypivalimidamide (963.21 mg, 8.29 mmol) at 0° C., the mixture was stirred at 50° C. for 2 h. The mixture was diluted with water (30 mL) and extracted with ethyl acetate (3×50 mL). The combined organic layers were dried over sodium sulphate and concentrated in vacuo. The reaction mixture was purified by column chromatography (silica gel, 100-200 mesh, 0-100% ethyl acetate in petroleum ether) affording (E)-ethyl 4-(((Z)-(1-amino-2,2-dimethylpropylidene)amino)oxy)-4-oxobut-2-enoate (1.14 g, 56%) as a white solid: ¹H NMR (400 MHz, Chloroform-d) δ 7.06-6.87 (m, 2H), 4.88-4.71 (m, 2H), 4.35-4.18 (m, 2H), 1.33 (t, J=7.1 Hz, 3H), 1.30-1.26 (m, 9H). LCMS Rt=0.636 min, m/z=242.1 [M+H]⁺.

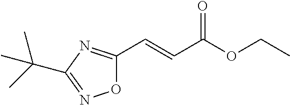 [SMILES: CCOC(=O)/C=C/c1nc(C(C)(C)C)no1]

### Step 4: (E)-ethyl 3-(3-(tert-butyl)-1,2,4-oxadiazol-5-yl)acrylate

To a solution (E)-ethyl 4-(((Z)-(1-amino-2,2-dimethylpropylidene)amino)oxy)-4-oxobut-2-enoate (1.14 g, 4.71 mmol) in dioxane (12 mL) was added 2,4,6-tripropyl-1,3,5,2,4,6-trioxatriphosphinane 2,4,6-trioxide (5.99 g, 9.41 mmol, 50% purity in ethyl acetate), the mixture was heated to 90° C. for 2 h. The mixture was diluted with water (20 mL) and extracted with ethyl acetate (3×20 mL). The combined organic layers were dried over sodium sulphate and concentrated in vacuo. The reaction mixture was purified by column chromatography (silica gel, 100-200 mesh, 0-100% ethyl acetate in petroleum ether) affording (E)-ethyl 3-(3-(tert-butyl)-1,2,4-oxadiazol-5-yl)acrylate (730 mg, 68%) as a colorless oil: ¹H NMR (400 MHz, Chloroform-d) δ 7.53-7.44 (m, 1H), 7.05-6.93 (m, 1H), 4.37-4.22 (m, 2H), 1.40 (s, 9H), 1.37-1.32 (m, 3H). LCMS Rt=0.907 min, m/z=224.1 [M+H]⁺.

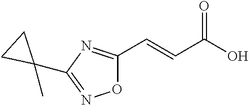

### Example B (Method 10): (E)-3-(3-(1-methylcyclopropyl)-1,2,4-oxadiazol-5-yl)acrylic acid

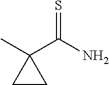

### Step 1: 1-methylcyclopropanecarbothioamide

To a solution of 1-methylcyclopropanecarboxamide (2 g, 20.18 mmol) in tetrahydrofuran (40 mL) was added potassium carbonate (2.79 g, 20.18 mmol) and lawesson reagent (8.16 g, 20.18 mmol), then the mixture was stirred at 80° C. for 2 h. The reaction mixture was quenched with a saturated solution of sodium bicarbonate (30 mL) and extracted with ethyl acetate (2×30 mL). The combined organic layers were dried over sodium sulphate and concentrated in vacuo. The resulting residue was purified by column chromatography (silica gel, 100-200 mesh, 50% ethyl acetate in petroleum ether) affording 1-methylcyclopropanecarbothioamide (1.8 g, 77.45%) as a white solid: ¹H NMR (400 MHz, Dimethyl sulfoxide-d6) δ 9.43 (br s, 1H), 8.69 (br s, 1H), 1.36-1.29 (m, 5H), 0.74 (q, J=3.6 Hz, 2H).

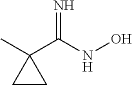

### Step 2: N-hydroxy-1-methylcyclopropanecarboximidamide

To a solution of 1-methylcyclopropanecarbothioamide (1.8 g, 15.63 mmol) in ethanol (20 mL) and water (10 mL) were added hydroxylammonium chloride (2.17 g, 31.25 mmol) and triethylamine (4.74 g, 6.52 mL), then the mixture was stirred at 80° C. for 12 h. The mixture was concentrated in vacuo. Then the resulting residue was diluted with water (30 mL) and extracted with ethyl acetate (2×100 mL). The combined organic layers were dried over sodium sulphate and concentrated under vacuo. The resulting residue was purified by column chromatography (silica gel, 100-200 mesh, 40% ethyl acetate in petroleum ether) affording N-hydroxy-1-methyl-cyclopropanecarboxamidine (1.53 g, 85.78%) as a yellow gum: ¹H NMR (400 MHz, DMSO-d6) δ 8.98-8.81 (m, 1H), 5.29-5.09 (m, 2H), 1.23-1.12 (m, 3H), 0.81 (br d, J=8.2 Hz, 2H), 0.50-0.34 (m, 2H).

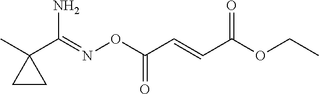 [SMILES: CCOC(=O)/C=C/C(=O)O/N=C(\N)C1(C)CC1]

### Step 3: (E)-ethyl 4-(((Z)-(amino(1-methylcyclopropyl)methylene)amino)oxy)-4-oxobut-2-enoate

The amide coupling reaction was prepared in a similar fashion to Method #4, Step 3. The crude product was purified by column chromatography (silica gel, 100-200 mesh, 30% ethyl acetate in petroleum ether) affording (E)-ethyl 4-(((Z)-(amino(1-methylcyclopropyl)methylene)amino)oxy)-4-oxobut-2-enoate (1.85 g, 57.45%) as a white solid. LCMS Rt=0.630 min, m/z=240.1 [M+H]⁺.

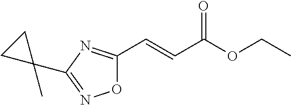 [SMILES: CCOC(=O)/C=C/c1nc(C2(C)CC2)no1]

### Step 4: (E)-ethyl 3-(3-(1-methylcyclopropyl)-1,2,4-oxadiazol-5-yl)acrylate

The cyclization reaction was prepared in a similar fashion to Method #4, Step 4. The resulting residue was purified by column chromatography (silica gel, 100-200 mesh, 20% ethyl acetate in petroleum ether) affording (E)-ethyl 3-(3-(1-methylcyclopropyl)-1,2,4-oxadiazol-5-yl)acrylate (1.2 g, 70.12%) as a yellow gum. LCMS Rt=0.820 min, m/z=222.1 [M+H]⁺.

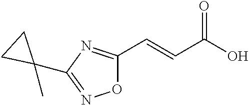

### Step 5: (E)-3-(3-(1-methylcyclopropyl)-1,2,4-oxadiazol-5-yl)acrylic acid

The hydrolysis reaction was prepared in a similar fashion to Method #1, Step 3. The reaction mixture was concentrated in vacuo affording (E)-3-(3-(1-methylcyclopropyl)-1,2,4-oxadiazol-5-yl)acrylic acid (800 mg, crude) as a white solid, used in next step without further purification. LCMS Rt=0.638 min, m/z=194.1 [M+H]⁺.

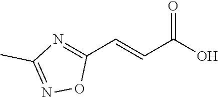

### Example C (Method 1). (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(3-(3-methyl-1,2,4-oxadiazol-5-yl)acryloyl)piperazin-2-yl)acetonitrile

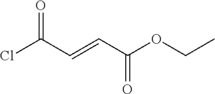

### Step 1: (E)-ethyl 4-chloro-4-oxobut-2-enoate

A mixture of (E)-4-ethoxy-4-oxo-but-2-enoic acid (13 g, 90.20 mmol), oxalyl dichloride (12.59 g, 99.22 mmol) and N,N-dimethylformaldehyde (659.27 mg, 9.02 mmol) in dichloromethane (80 mL) was degassed and purged with nitrogen (3 times), and then the mixture was stirred at 20° C. for 0.5 h. The reaction mixture was concentrated in vacuo affording (E)-ethyl 4-chloro-4-oxobut-2-enoate (14 g, crude) as a yellow oil used in the next step without further purification.

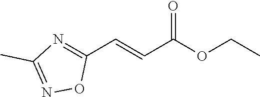

### Step 2: (E)-ethyl 3-(3-methyl-1,2,4-oxadiazol-5-yl)acrylate

A mixture of N′-hydroxyacetamidine (5 g, 67.49 mmol), ethyl (E)-4-chloro-4-oxo-but-2-enoate (12.07 g, 74.24 mmol) and pyridine (10.68 g, 134.99 mmol) in toluene (30 mL) was degassed and purged with nitrogen for 3 times, and then the mixture was stirred at 110° C. for 12 h. The reaction mixture was concentrated in vacuo and purified by column chromatography (silica gel, 100-200 mesh, 0-10% ethyl acetate in petroleum ether) affording (E)-ethyl 3-(3-methyl-1,2,4-oxadiazol-5-yl)acrylate (4.9 g, 85%) as a brown oil: ¹H NMR (400 MHz, Chloroform-d) δ 7.46-7.34, (m, 1H), 6.98-6.88 (m, 1H), 4.24 (q, J=7.13 Hz, 2H), 2.38 (s, 3H), 1.32-1.26 (m, 3H). LCMS Rt=0.383 min, m/z=182.1 [M+H]⁺.

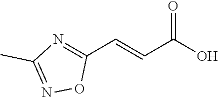

### Step 3: (E)-3-(3-methyl-1,2,4-oxadiazol-5-yl)acrylic acid

To a solution of ethyl (E)-3-(3-methyl-1,2,4-oxadiazol-5-yl)prop-2-enoate (1.08 g, 5.95 mmol) in tetrahydrofuran (20 mL) and water (10 mL) was added lithium hydroxide monohydrate (299.47 mg, 7.14 mmol) at 0° C., the mixture was stirred at 0° C. for 10 min. The reaction mixture was adjusted to pH=2 with hydrochloric acid (1 M, 5 mL) and extracted with ethyl acetate (3×30 mL). The combined organic layers were dried over sodium sulphate and concentrated in vacuo affording (E)-3-(3-methyl-1,2,4-oxadiazol-5-yl)acrylic acid (860 mg, crude) as a colorless oil used in the next step without further purification: ¹H NMR (400 MHz, Methanol-d4) δ 7.34 (d, J=16.1 Hz, 1H), 6.87 (d, J=16.1 Hz, 1H), 2.31 (s, 3H). LCMS Rt=0.200 min, m/z=154.0 [M+H]⁺.

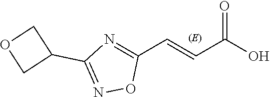 [SMILES: O=C(O)/C=C/c1nc(C2COC2)no1]

### Example D (Method 3): (E)-3-[3-(oxetan-3-yl)-1,2,4-oxadiazol-5-yl]prop-2-enoic acid

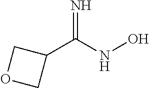

### Step 1: N-hydroxyoxetane-3-carboxamidine

The addition reaction was prepared in a similar fashion to Method #4, Step 2, the reaction mixture was concentrated in vacuo affording N-hydroxyoxetane-3-carboxamidine (1.26 g, crude) as a gray solid, used in next step without further purification. LCMS Rt=0.112 min, m/z=116.1 [M+H]⁺.

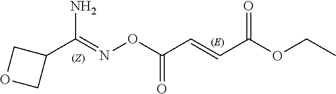 [SMILES: CCOC(=O)/C=C/C(=O)O/N=C(\N)C1COC1]

### Step 2: (E)-ethyl 4-(((Z)-(amino(oxetan-3-yl)methylene)amino)oxy)-4-oxobut-2-enoate

The amide coupling reaction was prepared in a similar fashion to Method #4, Step 3. The reaction mixture was concentrated in vacuo affording (E)-ethyl 4-(((Z)-(amino(oxetan-3-yl)methylene)amino)oxy)-4-oxobut-2-enoate (1.1 g, crude) as a white solid, used in next step without further purification. LCMS Rt=0.430 min, m/z=242.1 [M+H]⁺.

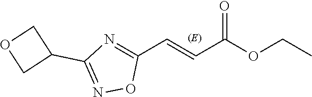 [SMILES: CCOC(=O)/C=C/c1nc(C2COC2)no1]

### Step 3: ethyl (E)-3-[3-(oxetan-3-yl)-1,2,4-oxadiazol-5-yl]prop-2-enoate

The cyclization reaction was prepared in a similar fashion to Method #4, Step 4. The reaction mixture was concentrated in vacuo affording ethyl (E)-3-[3-(oxetan-3-yl)-1,2,4-oxadiazol-5-yl]prop-2-enoate (860 mg, crude) as a brown oil, used in next step without further purification. LCMS Rt=0.671 min, m/z=224.1 [M+H]⁺.

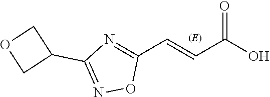 [SMILES: O=C(O)/C=C/c1nc(C2COC2)no1]

### Step 4: (E)-3-[3-(oxetan-3-yl)-1,2,4-oxadiazol-5-yl]prop-2-enoic acid

The hydrolysis reaction was prepared in a similar fashion to Method #1, Step 3. The reaction mixture was concentrated in vacuo affording (E)-3-[3-(oxetan-3-yl)-1,2,4-oxadiazol-5-yl]prop-2-enoic acid (570 mg, crude) as a yellow solid, used in next step without further purification. LCMS Rt=0.221 min, m/z=196.1 [M+H]⁺.

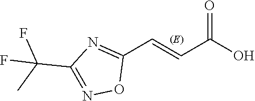

### Example E (Method 6): (E)-3-[3-(1,1-difluoroethyl)-1,2,4-oxadiazol-5-yl]prop-2-enoic acid

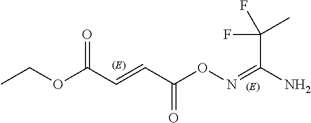 [SMILES: CCOC(=O)/C=C/C(=O)O/N=C(/N)C(C)(F)F]

### Step 1: (E)-ethyl 4-(((E)-(1-amino-2,2-difluoropropylidene)amino)oxy)-4-oxobut-2-enoate

The amide coupling reaction was prepared in a similar fashion to Method #4, Step 3. The reaction mixture was concentrated in vacuo affording (E)-ethyl 4-(((E)-(1-amino-2,2-difluoropropylidene)amino)oxy)-4-oxobut-2-enoate (3.6 g, crude) as a brown oil, used in the next step without further purification. LCMS Rt=0.510 min, m/z=250.1 [M+H]⁺.

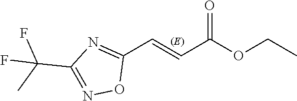 [SMILES: CCOC(=O)/C=C/c1nc(C(C)(F)F)no1]

### Step 2: ethyl (E)-3-[3-(1,1-difluoroethyl)-1,2,4-oxadiazol-5-yl]prop-2-enoate

The cyclization reaction was prepared in a similar fashion to Method #4, Step 4. The crude product was purified by column chromatography (silica gel, 100-200 mesh, 9% ethyl acetate in petroleum ether) affording ethyl (E)-3-[3-(1,1-difluoroethyl)-1,2,4-oxadiazol-5-yl]prop-2-enoate (650 mg, 43.78%) as a white solid. LCMS Rt=0.633 min, m/z=232.1 [M+H]⁺.

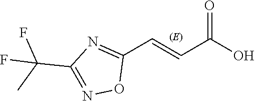

### Step 3: (E)-3-[3-(1,1-difluoroethyl)-1,2,4-oxadiazol-5-yl]prop-2-enoic acid

The hydrolysis reaction was prepared in a similar fashion to Method #1, Step 3.

The reaction mixture were concentrated in vacuo affording (E)-3-[3-(1,1-difluoroethyl)-1,2,4-oxadiazol-5-yl]prop-2-enoic acid (400 mg, crude) as a white solid used in the next step without further purification. LCMS Rt=0.545 min, m/z=204.0 [M+H]⁺.

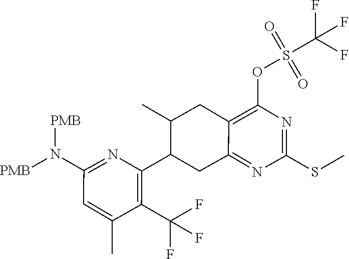

### Example F (Method 13): 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl trifluoromethanesulfonate

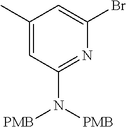

### Step 1: 6-bromo-N,N-bis(4-methoxybenzyl)-4-methylpyridin-2-amine

To a solution of sodium hydride (128.30 g, 2.67 mol, 60% purity) in N,N-dimethylformaldehyde (2000 mL) was added 6-bromo-4-methyl-pyridin-2-amine (100 g, 534.65 mmol) at 0° C. for 1 h, then 1-(chloromethyl)-4-methoxy-benzene (209.33 g, 1.34 mol) was added at 0° C. The resulting mixture was stirred at 25° C. for 11 h. The reaction mixture was quenched with a saturated solution of ammonium chloride (500 mL) at 0° C. and extracted with ethyl acetate (3×500 mL). The combined organic layers were dried over sodium sulphate and concentrated in vacuo. The crude product was triturated with water (50 mL), filtered, the filter cake was collected and concentrated in vacuo affording 6-bromo-N,N-bis(4-methoxybenzyl)-4-methylpyridin-2-amine (220 g, 96.29%) as a yellow solid: ¹H NMR (400 MHz, Chloroform-d) δ 7.07 (d, J=8.6 Hz, 4H), 6.80-6.73 (m, 4H), 6.51 (s, 1H), 6.07 (s, 1H), 4.55 (s, 4H), 3.71 (s, 6H), 2.04 (s, 3H). LCMS Rt=2.438 min, m/z=426.1 [M+H]⁺.

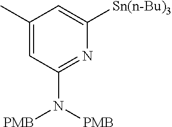

### Step 2: N,N-bis(4-methoxybenzyl)-4-methyl-6-(tributylstannyl)pyridin-2-amine

To a solution of 6-bromo-N,N-bis[(4-methoxyphenyl)methyl]-4-methyl-pyridin-2-amine (50 g, 117 mmol) in dioxane (4.0 L) was added lithium chloride (24.8 g, 582.02 mmol), tricyclohexylphosphane (6.56 g, 23.4 mmol), tricyclohexylphosphane (6.56 g, 23.4 mmol), (1E,4E)-1,5-diphenylpenta-1,4-dien-3-one;palladium (10.72 g, 11.7 mmol) and tributyl(tributylstannyl)stannane (169.68 g, 292.52 mmol). The mixture was stirred at 110° C. for 12 h under nitrogen atmosphere. The reaction mixture was filtered and the filtrate was concentrated to dryness in vacuo. The residue was purified by column chromatography (silica gel, 100-200 mesh, 0-10% ethyl acetate in petroleum ether) affording N,N-bis(4-methoxybenzyl)-4-methyl-6-(tributylstannyl)pyridin-2-amine (74 g, 99.21%) as a yellow oil. LCMS Rt=2.407 min, m/z=638.3 [M+H]⁺.

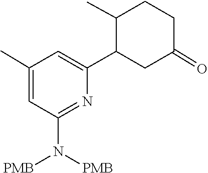

### Step 3: 3-(6-(bis(4-methoxybenzyl)amino)-4-methylpyridin-2-yl)-4-methylcyclohexanone

To a solution of N,N-bis[(4-methoxyphenyl)methyl]-4-methyl-6-tributylstannyl-pyridin-2-amine (38 g, 59.61 mmol) in tetrahydrofuran (2000 mL) was added chlororhodium;(1Z,5Z)-cycloocta-1,5-diene (2.94 g, 5.96 mmol, 0.1 eq), water (107.39 mg, 5.96 mmol) and 4-methylcyclohex-2-en-1-one (7.88 g, 71.53 mmol). The mixture was stirred at 60° C. for 12 h under nitrogen atmosphere. The reaction mixture was concentrated to dryness in vacuo. The residue was purified by column chromatography (silica gel, 100-200 mesh, 0-10% ethyl acetate in petroleum ether) affording 3-(6-(bis(4-methoxybenzyl)amino)-4-methylpyridin-2-yl)-4-methylcyclohexanone (35 g, 64.02%) as a yellow oil. LCMS Rt=1.824 min, m/z=458.3 [M+H]⁺.

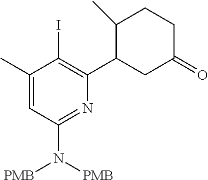

### Step 4: 3-(6-(bis(4-methoxybenzyl)amino)-3-iodo-4-methylpyridin-2-yl)-4-methylcyclohexanone

To a solution of 3-(6-(bis(4-methoxybenzyl)amino)-4-methylpyridin-2-yl)-4-methylcyclohexanone (34 g, 74.14 mmol) in N,N-dimethylformaldehyde (500 mL) was added N-iodo-succinimide (33.36 g, 148.28 mmol). The mixture was stirred at 25° C. for 3 h. The reaction mixture was concentrated to dryness in vacuo. The residue was purified by column chromatography (silica gel, 100-200 mesh, 0-10% ethyl acetate in petroleum ether) affording 3-(6-(bis(4-methoxybenzyl)amino)-3-iodo-4-methylpyridin-2-yl)-4-methylcyclohexanone (19 g, 43.85%) as a yellow oil. LCMS Rt=1.092 min, m/z=584.2 [M+H]⁺.

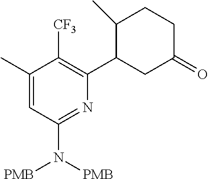

### Step 5: 3-(6-(bis(4-methoxybenzyl)amino)-4-methyl-3-(trifluoromethyl)pyridin-2-yl)-4-methylcyclohexanone

To a solution of 3-(6-(bis(4-methoxybenzyl)amino)-3-iodo-4-methylpyridin-2-yl)-4-methylcyclohexanone (15 g, 25.66 mmol) in N,N-dimethylformaldehyde (300 mL) was added cuprous iodide (14.66 g, 76.99 mmol) and methyl 2,2-difluoro-2-fluorosulfonyl-acetate (24.65 g, 128.32 mmol). The mixture was stirred at 90° C. for 2 h under nitrogen atmosphere. The reaction mixture was filtered and the filtrate was concentrated to dryness in vacuo. The residue was purified by column chromatography (silica gel, 100-200 mesh, 0-10% ethyl acetate in petroleum ether) affording 3-(6-(bis(4-methoxybenzyl)amino)-4-methyl-3-(trifluoromethyl)pyridin-2-yl)-4-methylcyclohexanone (12 g, 88.80%) as a yellow oil. LCMS Rt=0.875 min, m/z=526.2 [M+H]⁺.

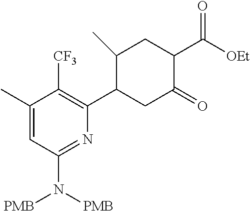

### Step 6: ethyl 4-(6-(bis(4-methoxybenzyl)amino)-4-methyl-3-(trifluoromethyl)pyridin-2-yl)-5-methyl-2-oxocyclohexanecarboxylate

To a solution of 3-(6-(bis(4-methoxybenzyl)amino)-4-methyl-3-(trifluoromethyl)pyridin-2-yl)-4-methylcyclohexanone (12 g, 22.78 mmol) in tetrahydrofuran (600 mL) was added lithium hexamethyldisilazane (1 M, 34.18 mL) at −78° C. under nitrogen atmosphere for 1 h, then ethyl cyanoformate (2.7 g, 27.34 mmol) was added at −78° C. The resulting mixture was stirred at -78° C. for 0.5 h under nitrogen atmosphere. The reaction mixture was quenched with a saturated solution of ammonium chloride (100 mL) and extracted with ethyl acetate (3×150 mL). The combined organic layers were dried over sodium sulphate and concentrated in vacuo affording ethyl 4-[6-[bis[(4-methoxyphenyl)methyl]amino]-4-methyl-3-(trifluoromethyl)-2-pyridyl]-5-methyl-2-oxo-cyclohexanecarboxylate (13.6 g, crude) as a yellow oil used in the next step without further purification. LCMS Rt=1.235 min, m/z=598.3 [M+H]⁺.

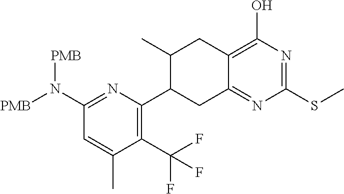

### Step 7: 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-ol

To a solution of ethyl 4-(6-(bis(4-methoxybenzyl)amino)-4-methyl-3-(trifluoromethyl)pyridin-2-yl)-5-methyl-2-oxocyclohexanecarboxylate (13 g, 21.72 mmol) in ethanol (1200 mL)/water (240 mL) was added sodium bicarbonate (45.61 g, 542.89 mmol) and 2-methylisothiourea;sulfuric acid (60.45 g, 217.15 mmol). The mixture was stirred at 50° C. for 12 h. The reaction mixture was filtered and the filtrate was concentrated to dryness in vacuo. The residue was purified by column chromatography (silica gel, 100-200 mesh, 0-10% ethyl acetate in petroleum ether) affording 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-ol (4.7 g, 34.65%) as a yellow solid. LCMS Rt=1.046 min, m/z=624.2 [M+H]⁺.

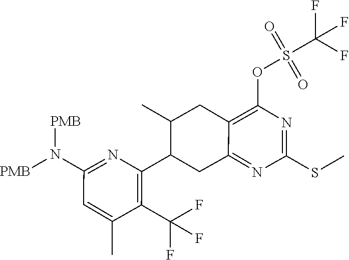

### Step 8: 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl trifluoromethanesulfonate

To a solution of 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-ol (1.5 g, 2.40 mmol) in dichloromethane (100 mL) was added triethylamine (970 mg, 9.60 mmol) and trifluoromethane anhydride (2.37 g, 8.40 mmol) at 0° C. under nitrogen atmosphere. The mixture was stirred at 25° C. for 1 h. The reaction mixture was quenched with a saturated solution of sodium bicarbonate (30 mL) at 0° C. and extracted with dichloromethane (3×100 mL). The combined organic layers were dried over sodium sulphate and concentrated in vacuo affording 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl trifluoromethanesulfonate (1.8 g, crude) as a yellow oil used in the next step without further purification. LCMS Rt=3.664 min, m/z=756.2 [M+H]⁺.

### EXEMPLARY COMPOUNDS

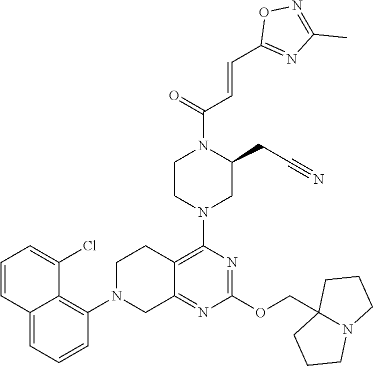 [SMILES: Cc1noc(/C=C/C(=O)N2CCN(c3nc(OCC45CCCN4CCC5)nc4c3CCN(c3cccc5cccc(Cl)c35)C4)C[C@@H]2CC#N)n1]

### Example 1. (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(3-(3-methyl-1,2,4-oxadiazol-5-yl)acryloyl)piperazin-2-yl)acetonitrile

The final compound was prepared in accordance with general Method 1, step 4. The crude product was purified by reverse phase prep-HPLC (column: Waters Xbridge Prep OBD C18 150*40 mm*10 um; mobile phase: [water (NH₄HCO₃)-ACN]; B %: 35%-65%,8 min) affording (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(3-(3-methyl-1,2,4-oxadiazol-5-yl)acryloyl)piperazin-2-yl)acetonitrile as a yellow oil: ¹H NMR (400 MHz, Acetonitrile-d3) δ 7.85 (d, J=8.1 Hz, 1H), 7.74-7.58 (m, 2H), 7.57-7.46 (m, 2H), 7.43-7.39 (m, 1H), 7.37 (d, J=6.6 Hz, 1H), 7.34 (s, 1H), 4.71 (br s, 1H), 4.26 (dd, J=7.6, 17.5 Hz, 1H), 4.16-3.99 (m, 2H), 3.96 (br d, J=2.1 Hz, 2H), 3.94-3.63 (m, 2H), 3.59-3.49 (m, 1H), 3.39-3.02 (m, 5H), 3.00-2.72 (m, 4H), 2.69-2.53 (m, 3H), 2.39 (s, 3H), 1.92-1.85 (m, 2H), 1.85-1.71 (m, 4H), 1.62-1.54 (m, 2H). LCMS Rt=2.924 min, m/z=693.3 [M+H]⁺.

LCMS (5 to 95% acetonitrile in water+0.1% trifluoroacetic acid over 6 mins) retention time 2.924 min, ESI+found [M+H]=693.3.

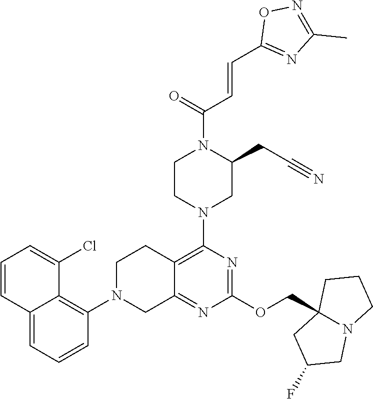 [SMILES: Cc1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1]

### Example 2. 2-((S)-4-(7-(8-chloronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1-((E)-3-(3-methyl-1,2,4-oxadiazol-5-yl)acryloyl)piperazin-2-yl)acetonitrile

The amide coupling reaction was prepared in a similar fashion to Method #1, Step 4. The crude product was purified by reverse phase prep-HPLC (column: Waters Xbridge BEH C18 100*30 mm*10 um; mobile phase: [water (NH₄HCO₃)-ACN]; B %: 55%-85%, 10 min) affording 2-((S)-4-(7-(8-chloronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1-((E)-3-(3-methyl-1,2,4-oxadiazol-5-yl)acryloyl)piperazin-2-yl)acetonitrile (13.07 mg, 12.95%) as a yellow solid: ¹H NMR (400 MHz, Acetonitrile-d3) δ 7.91-7.84 (m, 1H), 7.74-7.69 (m, 1H), 7.68-7.48 (m, 3H), 7.45-7.30 (m, 3H), 5.35-5.15 (m, 1H), 5.14-4.68 (m, 1H), 4.33-4.22 (m, 1H), 4.18-4.01 (m, 3H), 4.01-3.89 (m, 2H), 3.87-3.72 (m, 1H), 3.64-3.52 (m, 1H), 3.42-3.19 (m, 2H), 3.18-3.11 (m, 4H), 3.10-3.02 (m, 2H), 3.01-2.87 (m, 2H), 2.87-2.74 (m, 1H), 2.71-2.55 (m, 1H), 2.42 (s, 3H), 2.14-1.98 (m, 3H), 1.94-1.75 (m, 3H). LCMS Rt=3.338 min, m/z=711.3 [M+H]⁺. LCMS (5 to 95% acetonitrile in water+0.1% trifluoroacetic acid over 6 mins) retention time 3.338 min, ESI+found [M+H]=711.3.

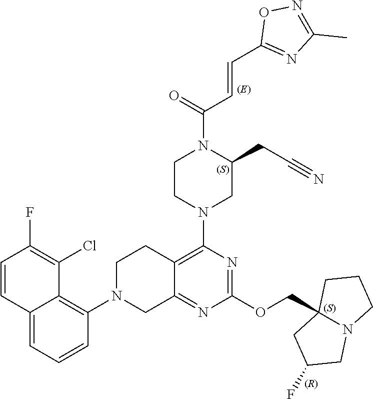

### Example 3. 2-((S)-4-(7-(8-chloro-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1-((E)-3-(3-methyl-1,2,4-oxadiazol-5-yl)acryloyl)piperazin-2-yl)acetonitrile

The amide coupling reaction was prepared in a similar fashion to Method #1, Step 4. The crude was purified by reverse phase HPLC (column: Waters Xbridge Prep OBD C18 150*40 mm*10 um; mobile phase: [water (NH₄HCO₃)-ACN]; B %: 50%-80%, 8 min) affording 2-((S)-4-(7-(8-chloro-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1-((E)-3-(3-methyl-1,2,4-oxadiazol-5-yl)acryloyl)piperazin-2-yl)acetonitrile (36.84 mg, 39.96%) as a yellow oil: ¹H NMR (400 MHz, Acetonitrile-d3) δ 7.89 (dd, J=5.8, 9.0 Hz, 1H), 7.73-7.68 (m, 1H), 7.66-7.56 (m, 1H), 7.53-7.45 (m, 1H), 7.45-7.32 (m, 3H), 5.35-5.01 (m, 2H), 4.79-4.47 (m, 1H), 4.24 (br dd, J=8.2, 17.4 Hz, 1H), 4.16-4.00 (m, 3H), 3.99-3.87 (m, 2H), 3.82-3.66 (m, 1H), 3.57-3.46 (m, 1H), 3.34 (br s, 1H), 3.22 (br d, J=4.6 Hz, 1H), 3.10 (br d, J=1.9 Hz, 3H), 3.07-2.99 (m, 2H), 2.95-2.83 (m, 2H), 2.80 (br d, J=7.4 Hz, 1H), 2.70-2.49 (m, 1H), 2.39 (s, 3H), 2.13 (br d, J=2.9 Hz, 1H), 2.07-1.99 (m, 2H), 1.87 (br d, J=16.1 Hz, 1H), 1.80 (br s, 2H). LCMS Rt=3.178 min, m/z=729.3 [M+H]⁺.

LCMS (5 to 95% acetonitrile in water+0.03% ammonium bicarbonate over 6 mins) retention time 3.178 min, ESI+found [M+H]=729.3.

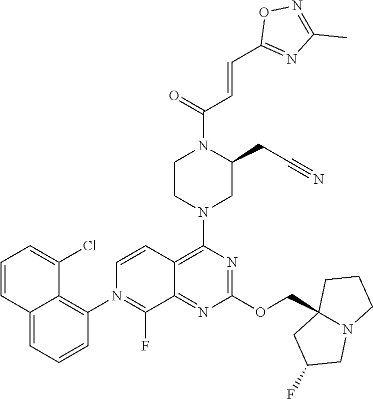

### Example 4. 2-((S)-4-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(3-methyl-1,2,4-oxadiazol-5-yl)acryloyl)piperazin-2-yl)acetonitrile

The amide coupling reaction was prepared in a similar fashion to Method #1, Step 4.

The crude product was purified by reverse phase prep-HPLC (column: Waters Xbridge Prep OBD C18 150*40 mm*10 um; mobile phase: [water (NH₄HCO₃)-ACN]; B %: 40%-70%, 8 min) affording 2-((S)-4-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(3-methyl-1,2,4-oxadiazol-5-yl)acryloyl)piperazin-2-yl)acetonitrile (28.28 mg, 32.83%) as a yellow oil: ¹H NMR (400 MHz, Acetonitrile-d3) δ 9.13 (s, 1H), 8.17-8.11 (m, 1H), 8.03 (d, J=8.3 Hz, 1H), 7.77-7.57 (m, 4H), 7.55-7.50 (m, 1H), 7.46-7.36 (m, 1H), 5.40-5.17 (m, 1H), 5.12-4.79 (m, 1H), 4.60-4.45 (m, 2H), 4.26-4.21 (m, 1H), 4.18-4.04 (m, 2H), 4.01-3.55 (m, 3H), 3.20-3.12 (m, 2H), 3.08 (s, 1H), 3.01-2.82 (m, 3H), 2.40 (s, 3H), 2.19 (br s, 1H), 2.12 (br s, 1H), 2.09-2.04 (m, 1H), 1.93-1.80 (m, 3H). LCMS Rt=3.071 min, m/z=725.2 [M+H]⁺.

LCMS (5 to 95% acetonitrile in water+0.03% ammonium bicarbonate over 6 mins) retention time 3.071 min, ESI+found [M+H]=725.2.

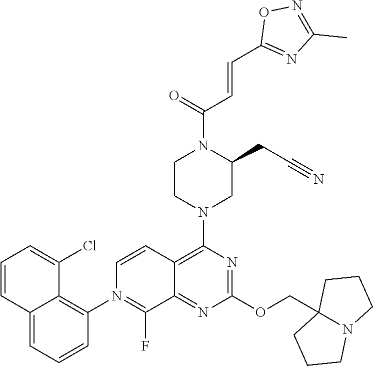

### Example 5: (S,E)-2-(4-(7-(8-chloronaphthalen-1-yl)-8-fluoro-2-((tetrahydro-1H-pyrrolizin-7a(5H)-yl)methoxy)pyrido[4,3-d]pyrimidin-4-yl)-1-(3-(3-methyl-1,2,4-oxadiazol-5-yl)acryloyl)piperazin-2-yl)acetonitrile

The amide coupling reaction was prepared in a similar fashion to Method #1, Step 4.

The crude product was purified by reverse phase prep-HPLC (column: Waters Xbridge BEH C18 100*30 mm*10 um; mobile phase: [water (NH₄HCO₃)-ACN]; B %: 35%-65%,10 min) affording (S,E)-2-(4-(7-(8-chloronaphthalen-1-yl)-8-fluoro-2-((tetrahydro-1H-pyrrolizin-7a(5H)-yl)methoxy)pyrido[4,3-d]pyrimidin-4-yl)-1-(3-(3-methyl-1,2,4-oxadiazol-5-yl)acryloyl)piperazin-2-yl)acetonitrile (27.75 mg, 26.98%) as a yellow solid: ¹H NMR (400 MHz, Acetonitrile-d3) δ 9.12 (s, 1H), 8.13 (dd, J=1.1, 8.1 Hz, 1H), 8.02 (d, J=8.3 Hz, 1H), 7.72-7.67 (m, 1H), 7.67-7.56 (m, 3H), 7.55-7.49 (m, 1H), 7.44-7.35 (m, 1H), 4.83 (br s, 1H), 4.56-4.41 (m, 2H), 4.22-4.06 (m, 3H), 3.96-3.72 (m, 2H), 3.70-3.44 (m, 1H), 3.39-3.08 (m, 1H), 3.02-2.93 (m, 3H), 2.62 (td, J=6.7, 9.9 Hz, 2H), 2.42-2.38 (m, 3H), 2.00-1.97 (m, 1H), 1.91 (br d, J=2.5 Hz, 5H), 1.69-1.60 (m, 2H). LCMS Rt=0.968 min, m/z=707.3 [M+H]⁺.

LCMS (5 to 95% acetonitrile in water+0.03% ammonium bicarbonate over 6 mins) retention time 0.968 min, ESI+found [M+H]=707.3.

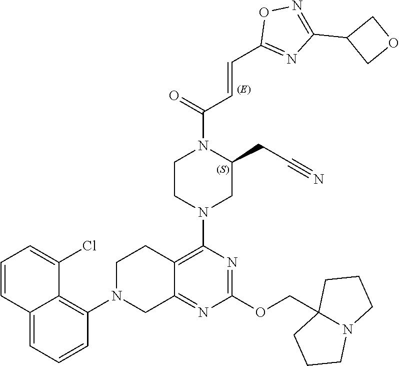

### Example 6: (S,E)-2-(4-(7-(8-chloronaphthalen-1-yl)-2-((tetrahydro-1H-pyrrolizin-7a(5H)-yl)methoxy)-5,6,7,8-tetrahydropyrido[3,4-d]pyrimidin-4-yl)-1-(3-(3-(oxetan-3-yl)-1,2,4-oxadiazol-5-yl)acryloyl)piperazin-2-yl)acetonitrile

The amide coupling reaction was prepared in a similar fashion to Method #1, Step 4. The crude was purified by reverse phase HPLC (column: Waters Xbridge Prep OBD C18 150*40 mm*10 um; mobile phase: [water (NH₄HCO₃)-ACN]; B %: 30%-60%, 8 min) affording (S,E)-2-(4-(7-(8-chloronaphthalen-1-yl)-2-((tetrahydro-1H-pyrrolizin-7a(5H)-yl)methoxy)-5,6,7,8-tetrahydropyrido[3,4-d]pyrimidin-4-yl)-1-(3-(3-(oxetan-3-yl)-1,2,4-oxadiazol-5-yl)acryloyl)piperazin-2-yl)acetonitrile (29.22 mg, 20.95%) as a white solid: ¹H NMR (400 MHz, Acetonitrile-d3) δ 7.87 (d, J=8.0 Hz, 1H), 7.82-7.66 (m, 2H), 7.60-7.54 (m, 1H), 7.54-7.48 (m, 1H), 7.47-7.31 (m, 3H), 5.17-5.06 (m, 0.5H), 5.00 (dd, J=6.1, 8.4 Hz, 2H), 4.84 (br t, J=5.6 Hz, 2H), 4.76 (br s, 0.5H), 4.59-4.44 (m, 1H), 4.28 (br dd, J=7.1, 17.4 Hz, 1H), 4.15-4.02 (m, 2H), 4.01-3.90 (m, 3H), 3.86-3.72 (m, 1H), 3.61-3.50 (m, 1H), 3.42-3.31 (m, 1H), 3.27-3.05 (m, 4H), 3.03-2.92 (m, 3H), 2.90-2.77 (m, 1H), 2.71-2.56 (m, 3H), 1.92 (br dd, J=5.9, 11.9 Hz, 2H), 1.86-1.73 (m, 4H), 1.65-1.56 (m, 2H). LCMS Rt=2.980 min, m/z=735.3 [M+H]⁺.

LCMS (5 to 95% acetonitrile in water+0.03% ammonium bicarbonate over 6 mins) retention time 2.980 min, ESI+found [M+H]=735.3.

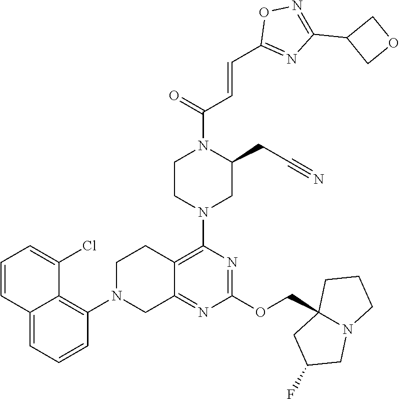 [SMILES: N#CC[C@H]1CN(c2nc(OC[C@@]34CCCN3C[C@H](F)C4)nc3c2CCN(c2cccc4cccc(Cl)c24)C3)CCN1C(=O)/C=C/c1nc(C2COC2)no1]

### Example 7: 2-((S)-4-(7-(8-chloronaphthalen-1-yl)-2-(((2R,7aS)-2-fluorohexahydro-1H-pyrrolizin-7a-yl)meth oxy)-5, 6, 7,8-tetrahydropyrido[3,4-d]pyrimidin-4-yl)-1-((E)-3-(3-(oxetan-3-yl)-1,2,4-oxadiazol-5-yl)acryloyl)piperazin-2-yl)acetonitrile

The amide coupling reaction was prepared in a similar fashion to Method #1, Step 4. The reaction mixture was purified by reverse phase HPLC (column: Waters Xbridge BEH C18 100*30 mm*10 um; mobile phase: [water (NH₄HCO₃)-ACN]; B %: 40%-70%, 8 min) affording 2-((S)-4-(7-(8-chloronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1-((E)-3-(3-(oxetan-3-yl)-1,2,4-oxadiazol-5-yl)acryloyl)piperazin-2-yl)acetonitrile (20.24 mg, 16%) as a yellow amorphous solid: ¹H NMR (400 MHz, Acetonitrile-d3) δ 7.88 (d, J=8.1 Hz, 1H), 7.83-7.67 (m, 2H), 7.61-7.56 (m, 1H), 7.55-7.49 (m, 1H), 7.46 (br d, J=7.8 Hz, 1H), 7.44-7.32 (m, 2H), 5.36-5.18 (m, 1H), 5.12 (br dd, J=1.4, 7.8 Hz, 0.5H), 5.00 (dd, J=6.3, 8.3 Hz, 2H), 4.87-4.81 (m, 2H), 4.80-4.70 (m, 0.5H), 4.59-4.44 (m, 1H), 4.33-4.23 (m, 1H), 4.18-4.03 (m, 3H), 4.03-3.91 (m, 2H), 3.87-3.71 (m, 1H), 3.63-3.50 (m, 1H), 3.42-3.28 (m, 1H), 3.25-3.03 (m, 7H), 3.02-2.77 (m, 3H), 2.73-2.53 (m, 1H), 2.17 (br s, 1H), 2.12-2.03 (m, 2H), 1.94-1.81 (m, 3H). LCMS Rt=3.118 min, m/z=753.3 [M+H]⁺.

LCMS (5 to 95% acetonitrile in water+0.03% ammonium bicarbonate over 6 mins) retention time 3.118 min, ESI+found [M+H]=753.3.

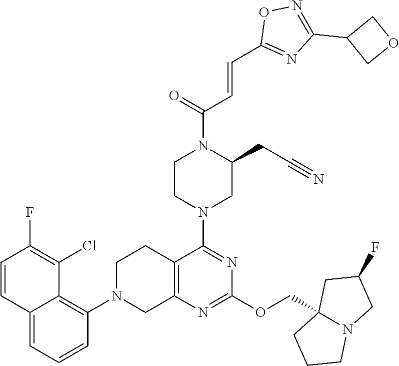 [SMILES: N#CC[C@H]1CN(c2nc(OC[C@@]34CCCN3C[C@H](F)C4)nc3c2CCN(c2cccc4ccc(F)c(Cl)c24)C3)CCN1C(=O)/C=C/c1nc(C2COC2)no1]

### Example 8: 2-((S)-4-(7-(8-chloro-7-fluoronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1-((E)-3-(3-(oxetan-3-yl)-1,2,4-oxadiazol-5-yl)acryloyl)piperazin-2-yl)acetonitrile

The amide coupling reaction was prepared in a similar fashion to Method #1, Step 4. The crude product was purified by reverse phase HPLC (column: Waters Xbridge BEH C18 100*30 mm*10 um; mobile phase: [water(ammonium bicarbonate)-acetonitrile]; B %: 35%-65%,10 min) affording 2-((S)-4-(7-(8-chloro-7-fluoronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1-((E)-3-(3-(oxetan-3-yl)-1,2,4-oxadiazol-5-yl)acryloyl)piperazin-2-yl)acetonitrile (5.91 mg, 6.11%) as a yellow solid: ¹H NMR (400 MHz, Acetonitrile-d3) δ 7.89 (dd, J=5.6, 9.0 Hz, 1H), 7.70 (br dd, J=3.5, 8.3 Hz, 2H), 7.53-7.35 (m, 4H), 5.32-5.13 (m, 1H), 5.03-4.94 (m, 2H), 4.81 (br t, J=5.3 Hz, 2H), 4.44 (br d, J=3.5 Hz, 1H), 4.29-4.18 (m, 1H), 4.11-4.02 (m, 2H), 4.00-3.89 (m, 2H), 3.87-3.68 (m, 2H), 3.64-3.28 (m, 3H), 3.16-3.01 (m, 7H), 2.92-2.76 (m, 3H), 2.68-2.56 (m, 1H), 2.06-2.00 (m, 2H), 1.89-1.76 (m, 4H). LCMS Rt=3.102 min, m/z=771.3 [M+H]⁺.

LCMS (5 to 95% acetonitrile in water+0.1% ammonium bicarbonate acid over 6 mins) retention time 3.102 min, ESI+found [M+H]=771.3.

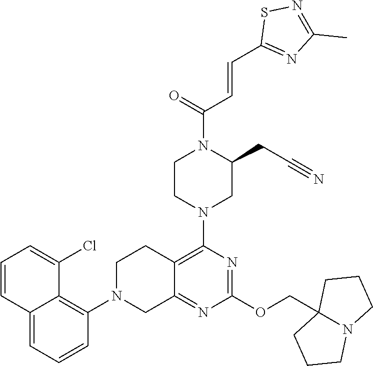 [SMILES: Cc1nsc(/C=C/C(=O)N2CCN(c3nc(OCC45CCCN4CCC5)nc4c3CCN(c3cccc5cccc(Cl)c35)C4)C[C@@H]2CC#N)n1]

### Example 9 (Method 5): (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(3-(3-methyl-1,2,4-thiadiazol-5-yl)acryloyl)piperazin-2-yl)acetonitrile

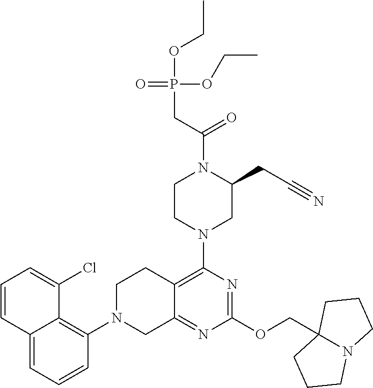 [SMILES: CCOP(=O)(CC(=O)N1CCN(c2nc(OCC34CCCN3CCC4)nc3c2CCN(c2cccc4cccc(Cl)c24)C3)C[C@@H]1CC#N)OCC]

### Step 1: (S)-diethyl (2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-2-(cyanomethyl)piperazin-1-yl)-2-oxoethyl)phosphonate

The amide coupling reaction was prepared in a similar fashion to Method #1, Step 4. The crude product was purified by column chromatography (silica gel, 100-200 mesh, 0-100% methanol in dichloromethane) affording (S)-diethyl (2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-2-(cyanomethyl)piperazin-1-yl)-2-oxoethyl)phosphonate (400 mg, 68%) as a brown solid. LCMS Rt=2.252 min, m/z=735.3 [M+H]⁺.

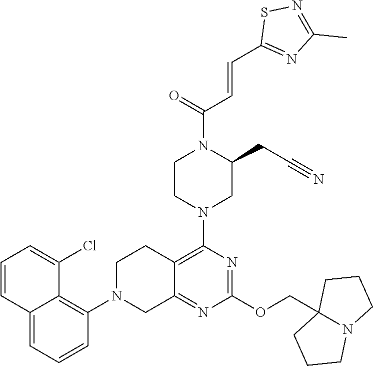 [SMILES: Cc1nsc(/C=C/C(=O)N2CCN(c3nc(OCC45CCCN4CCC5)nc4c3CCN(c3cccc5cccc(Cl)c35)C4)C[C@@H]2CC#N)n1]

### Step 2: (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(3-(3-methyl-1,2,4-thiadiazol-5-yl)acryloyl)piperazin-2-yl)acetonitrile

To a solution of (S)-diethyl (2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-2-(cyanomethyl)piperazin-1-yl)-2-oxoethyl)phosphonate (80 mg, 108.66 umol), lithium chloride (9.21 mg, 217.32 umol) and N,N-diisopropylethylamine (42.13 mg, 325.98 umol) in acetonitrile (2 mL) was added 3-methyl-1,2,4-thiadiazole-5-carbaldehyde (27.85 mg, 217.32 umol). The mixture was stirred at 25° C. for 1 h. The reaction mixture was filtered. The filtrate was purified by reverse phase HPLC (column: Waters Xbridge Prep OBD C18 150*40 mm*10 um; mobile phase: [water (NH₄HCO₃)-ACN]; B %: 35%-65%,8 min) affording (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(3-(3-methyl-1,2,4-thiadiazol-5-yl)acryloyl)piperazin-2-yl)acetonitrile (25.47 mg, 32%) as a white solid: ¹H NMR (400 MHz, Acetonitrile-d3) δ 7.92-7.87 (m, 1H), 7.77 (br s, 1H), 7.73 (dd, J=4.0, 7.7 Hz, 1H), 7.65-7.49 (m, 3H), 7.48-7.33 (m, 2H), 5.33-5.08 (m, 1H), 4.85-4.53 (m, 1H), 4.36-4.20 (m, 1H), 4.17-3.97 (m, 4H), 3.89-3.69 (m, 1H), 3.65-3.49 (m, 1H), 3.37 (br s, 1H), 3.29-3.04 (m, 4H), 3.02-2.88 (m, 3H), 2.86-2.76 (m, 1H), 2.74-2.54 (m, 6H), 1.94 (br s, 2H), 1.89-1.72 (m, 4H), 1.69-1.57 (m, 2H). LCMS Rt=3.037 min, m/z=709.3 [M+H].

LCMS (5 to 95% acetonitrile in water+0.03% ammonium bicarbonate over 6 mins) retention time 3.037 min, ESI+found [M+H]=709.3.

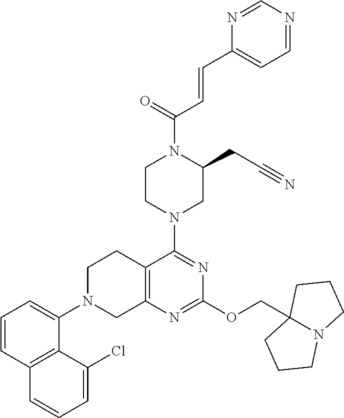 [SMILES: N#CC[C@H]1CN(c2nc(OCC34CCCN3CCC4)nc3c2CCN(c2cccc4cccc(Cl)c24)C3)CCN1C(=O)/C=C/c1ccncn1]

### Example 10: (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(3-(pyrimidin-4-yl)acryloyl)piperazin-2-yl)acetonitrile

The Horner-Wadsworth-Emmons reaction was prepared in a similar fashion to Method #5, Step 2. The crude was purified by reverse phase HPLC (column: Waters Xbridge Prep OBD C18 150*40 mm*10 um; mobile phase: [water(NH₄HCO₃)-ACN]; B %: 25%-55%,8 min) affording (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(3-(pyrimidin-4-yl)acryloyl)piperazin-2-yl)acetonitrile (12.34 mg, 15%) as a yellow solid: ¹H NMR (400 MHz, Acetonitrile-d3) δ 9.19 (br s, 1H), 8.83-8.77 (m, 1H), 7.90-7.82 (m, 1H), 7.81-7.67 (m, 2H), 7.57 (br d, J=7.0 Hz, 2H), 7.55-7.48 (m, 2H), 7.44-7.38 (m, 1H), 7.38-7.30 (m, 1H), 5.32-5.07 (m, 1H), 4.83-4.51 (m, 1H), 4.28 (dd, J=6.8, 17.4 Hz, 1H), 4.15-4.04 (m, 2H), 3.99 (d, J=3.3 Hz, 2H), 3.85-3.71 (m, 1H), 3.61-3.51 (m, 1H), 3.40-3.18 (m, 2H), 3.16-3.03 (m, 3H), 3.01-2.92 (m, 3H), 2.90-2.76 (m, 1H), 2.70-2.56 (m, 3H), 1.95-1.87 (m, 2H), 1.85-1.73 (m, 4H), 1.64-1.55 (m, 2H). LCMS Rt=2.283 min, m/z=689.3 [M+H]⁺.

LCMS (5 to 95% acetonitrile in water+0.03% ammonium bicarbonate over 6 mins) retention time 2.283 min, ESI+found [M+H]=689.3.

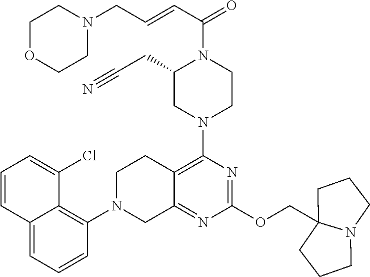 [SMILES: N#CC[C@H]1CN(c2nc(OCC34CCCN3CCC4)nc3c2CCN(c2cccc4cccc(Cl)c24)C3)CCN1C(=O)/C=C/CN1CCOCC1]

### Example 11: (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(4-morpholinobut-2-enoyl)piperazin-2-yl)acetonitrile

The Horner-Wadsworth-Emmons reaction was prepared in a similar fashion to Method #5, Step 2. The crude product was purified by reverse phase HPLC (column: Waters Xbridge Prep OBD C18 150*40 mm*10 um; mobile phase: [water(ammonium bicarbonate)-acetonitrile]; B %: 25%-55%, 8 min) affording (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(4-morpholinobut-2-enoyl)piperazin-2-yl)acetonitrile (25.93 mg, 16.20%) as a white solid: ¹H NMR (400 MHz, Acetonitrile-d3) δ 7.87 (d, J=8.2 Hz, 1H), 7.74-7.67 (m, 1H), 7.60-7.47 (m, 2H), 7.45-7.30 (m, 2H), 6.76 (td, J=5.7, 14.9 Hz, 1H), 6.69-6.50 (m, 1H), 5.25-4.86 (m, 1H), 4.73-4.38 (m, 1H), 4.27 (dd, J=7.1, 17.5 Hz, 1H), 4.20-3.88 (m, 5H), 3.86-3.71 (m, 1H), 3.68-3.51 (m, 6H), 3.31 (br d, J=10.5 Hz, 1H), 3.23 (br s, 1H), 3.17-3.07 (m, 4H), 3.07-2.90 (m, 4H), 2.88 (br d, J=3.1 Hz, 1H), 2.80-2.71 (m, 1H), 2.69-2.60 (m, 3H), 2.59-2.51 (m, 1H), 1.96-1.90 (m, 2H), 1.89-1.75 (m, 4H), 1.70-1.58 (m, 2H). LCMS Rt=2.835 min, m/z=710.4 [M+H]⁺. LCMS (5 to 95% acetonitrile in water+0.1% trifluoroacetic acid over 6 mins) retention time 2.835 min, ESI+found [M+H]=710.4.

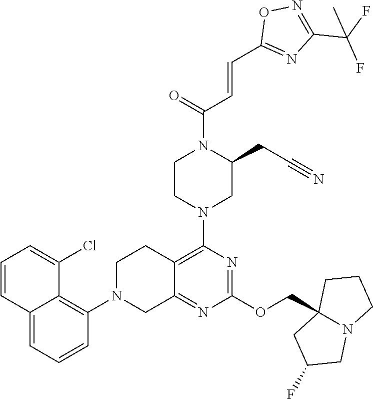 [SMILES: CC(F)(F)c1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1]

### Example 12 2-((S)-4-(7-(8-chloronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1-((E)-3-(3-(1,1-difluoroethyl)-1,2,4-oxadiazol-5-yl)acryloyl)piperazin-2-yl)acetonitrile

The amide coupling reaction was prepared in a similar fashion to Method #1, Step 4. The crude was purified by reverse phase HPLC (column: Waters Xbridge Prep OBD C18 150*40 mm*10 um; mobile phase: [water(NH₄HCO₃)-ACN]; B %: 50%-85%,8 min) affording 2-((S)-4-(7-(8-chloronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1-((E)-3-(3-(1,1-difluoroethyl)-1,2,4-oxadiazol-5-yl)acryloyl)piperazin-2-yl)acetonitrile (21.99 mg, 18%) as a yellow amorphous solid: ¹H NMR (400 MHz, Acetonitrile-d3) δ 7.87-7.77 (m, 1H), 7.77-7.59 (m, 2H), 7.58-7.41 (m, 3H), 7.41-7.27 (m, 2H), 5.34-5.11 (m, 1H), 4.77-4.45 (m, 1H), 4.24 (br dd, J=7.1, 17.4 Hz, 1H), 4.15-3.85 (m, 5H), 3.84-3.66 (m, 1H), 3.61-3.42 (m, 1H), 3.39-3.15 (m, 2H), 3.14-2.97 (m, 6H), 2.96-2.72 (m, 3H), 2.70-2.48 (m, 1H), 2.11-1.94 (m, 6H), 1.89-1.73 (m, 3H). LCMS Rt=3.522 min, m/z=761.3 [M+H]⁺.

LCMS (5 to 95% acetonitrile in water+0.03% ammonium bicarbonate over 6 mins) retention time 3.522 min, ESI+found [M+H]=761.3.

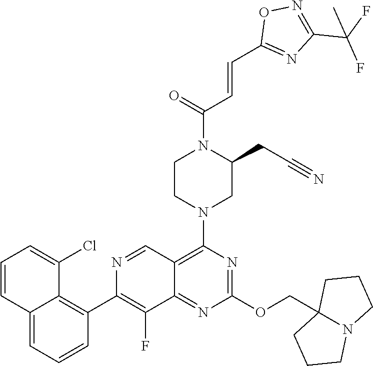 [SMILES: CC(F)(F)c1noc(/C=C/C(=O)N2CCN(c3nc(OCC45CCCN4CCC5)nc4c(F)c(-c5cccc6cccc(Cl)c56)ncc34)C[C@@H]2CC#N)n1]

### Example 13: (S,E)-2-(4-(7-(8-chloronaphthalen-1-yl)-8-fluoro-2-((tetrahydro-1H-pyrrolizin-7a(5H)-yl)methoxy)pyrido[4,3-d]pyrimidin-4-yl)-1-(3-(3-(1,1-difluoroethyl)-1,2,4-oxadiazol-5-yl)acryloyl)piperazin-2-yl)acetonitrile

The amide coupling reaction was prepared in a similar fashion to Method #1, Step 4.

The crude product was purified by reverse phase HPLC (column: Phenomenex Luna C18 75*30 mm*3 um; mobile phase: [water (formic acid)-ACN]; B %: 20%-50%, 8 min) affording (S,E)-2-(4-(7-(8-chloronaphthalen-1-yl)-8-fluoro-2-((tetrahydro-1H-pyrrolizin-7a(5H)-yl)methoxy)pyrido[4,3-d]pyrimidin-4-yl)-1-(3-(3-(1,1-difluoroethyl)-1,2,4-oxadiazol-5-yl)acryloyl)piperazin-2-yl)acetonitrile (5.09 mg, 5.43%, formate salt) as a white solid: ¹H NMR (400 MHz, Acetonitrile-d3) δ 9.11 (br s, 1H), 8.11 (br d, J=8.1 Hz, 1H), 8.01 (br d, J=8.1 Hz, 1H), 7.74-7.64 (m, 2H), 7.63-7.57 (m, 2H), 7.54-7.39 (m, 2H), 5.13-4.78 (m, 1H), 4.59-4.36 (m, 3H), 4.25 (br s, 2H), 4.08 (br d, J=10.0 Hz, 1H), 3.97-3.60 (m, 3H), 3.17-3.04 (m, 3H), 3.01-2.88 (m, 2H), 2.12 (s, 1H), 2.10-2.06 (m, 2H), 2.05-1.97 (m, 3H), 1.86 (br d, J=5.9 Hz, 3H), 1.70 (br dd, J=6.3, 11.3 Hz, 2H). LCMS Rt=2.366 min, m/z=757.2 [M+H]⁺.

LCMS (5 to 95% acetonitrile in water+0.1% trifluoroacetic acid over 6 mins) retention time 2.366 min, ESI+found [M+H]=757.2.

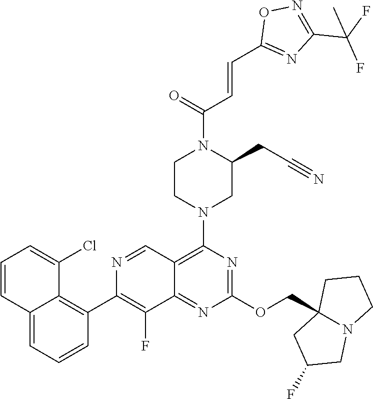 [SMILES: CC(F)(F)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1]

### Example 14: 2-((S)-4-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(3-(1,1-difluoroethyl)-1,2,4-oxadiazol-5-yl)acryloyl)piperazin-2-yl)acetonitrile

The amide coupling reaction was prepared in a similar fashion to Method #1, Step 4.

The crude product was purified by reverse phase HPLC (column: Waters Xbridge Prep OBD C18 150*40 mm*10 um; mobile phase: [water (NH₄HCO₃)-ACN]; B %: 40%-70%, 8 min) affording 2-((S)-4-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(3-(1,1-difluoroethyl)-1,2,4-oxadiazol-5-yl)acryloyl)piperazin-2-yl)acetonitrile (13.58 mg, 11.82%) as a yellow solid: ¹H NMR (400 MHz, Acetonitrile-d3) δ 9.16 (s, 1H), 8.16 (d, J=8.1 Hz, 1H), 8.05 (d, J=8.1 Hz, 1H), 7.92-7.61 (m, 4H), 7.57-7.42 (m, 2H), 5.44-5.19 (m, 1H), 5.15-4.76 (m, 1H), 4.61-4.42 (m, 2H), 4.35-4.24 (m, 1H), 4.21-4.05 (m, 2H), 4.01-3.53 (m, 3H), 3.26-3.10 (m, 3H), 3.04-2.86 (m, 3H), 2.22 (br s, 2H), 2.12-2.07 (m, 3H), 1.95-1.78 (m, 4H). LCMS Rt=2.359 min, m/z=775.2 [M+H]⁺.

LCMS (5 to 95% acetonitrile in water+0.1% trifluoroacetic acid over 6 mins) retention time 2.359 min, ESI+found [M+H]=775.2.

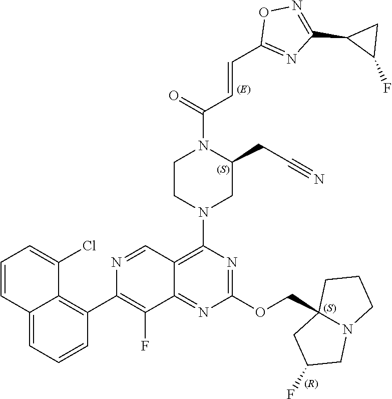

### Example 15 (Method 9): 2-((S)-4-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(3-((1R,2S)-2-fluorocyclopropyl)-1,2,4-oxadiazol-5-yl)acryloyl)piperazin-2-yl)acetonitrile

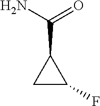

### Step 1: (1S,2R)-2-fluorocyclopropanecarboxamide

To a solution of (1S,2R)-2-fluorocyclopropanecarboxylic acid (10 g, 96.08 mmol) in tetrahydrofuran (100 mL) was added triethylamine (34.03 g, 336.28 mmol). To this well stirred mixture was added dropwise isobutyl carbonochloridate (19.68 g, 144.12 mmol) at 0° C., then the mixture was stirred at 20° C. for 0.5 h. Then ammonium chloride (1 M, 192.16 mL) was added dropwise to the above solution and stirred at 20° C. for 12 h. The reaction mixture was diluted with water (15 mL) and extracted with dichloromethane (2×50 mL). The combined organic layers were dried over sodium sulphate and concentrated under vacuo. The resulting residue was purified by column chromatography (silica gel, 100-200 mesh, 90-100% ethyl acetate in petroleum ether) affording (1S,2R)-2-fluorocyclopropanecarboxamide (6.5 g, 65.62%) as a white solid: ¹H NMR (400 MHz, Methanol-d4) δ 4.85-4.60 (m, 1H), 2.15-2.00 (m, 1H), 1.47-1.31 (m, 1H), 1.23 (qd, J=6.5, 12.8 Hz, 1H).

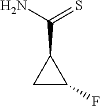

### Step 2: (1R,2R)-2-fluorocyclopropanecarbothioamide

The sulfamide formation was prepared in a similar fashion to Method #10, Step 1, The crude residue was purified by column chromatography (silica gel, 100-200 mesh, 0-20% ethyl acetate in petroleum ether) affording (1R,2R)-2-fluorocyclopropanecarbothioamide (3 g, 86.52%) as a white solid: ¹H NMR (400 MHz, Methanol-d4) δ 4.88 (ddd, J=1.5, 3.4, 6.1 Hz, 0.5H), 4.72 (ddd, J=1.4, 3.5, 6.0 Hz, 0.5H), 2.53-2.39 (m, 1H), 1.64-1.56 (m, 1H), 1.56-1.43 (m, 1H).

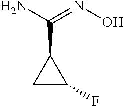

### Step 3: (1S,2R,E)-2-fluoro-N′-hydroxycyclopropanecarboximidamide

The hydroxyl amidine formation was prepared in a similar fashion to Method #10, Step 2, the crude product was purified by flash column (silica gel, 100-200 mesh, 0-20% ethyl acetate in petroleum ether) affording (1S,2R,E)-2-fluoro-N′-hydroxycyclopropanecarboximidamide (2.6 g, 87.44%) as a yellow gum: ¹H NMR (400 MHz, Methanol-d4) δ=4.84-4.63 (m, 1H), 1.91 (dddd, J=1.9, 7.3, 11.3, 18.5 Hz, 1H), 1.36-1.23 (m, 1H), 1.14 (qd, J=6.9, 11.2 Hz, 1H).

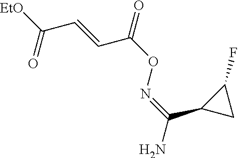 [SMILES: CCOC(=O)/C=C/C(=O)O/N=C(/N)[C@@H]1C[C@H]1F]

### Step 4: (E)-ethyl 4-(((E)-(amino((1S,2R)-2-fluorocyclopropyl)methylene)amino)oxy)-4-oxobut-2-enoate

The amide coupling reaction was prepared in a similar fashion to Method #4, Step 3.

The crude product was purified by flash column (silica gel, 100-200 mesh, 0-20% ethyl acetate in petroleum ether) affording (E)-ethyl 4-(((E)-(amino((1S,2R)-2-fluorocyclopropyl)methylene)amino)oxy)-4-oxobut-2-enoate (3.6 g, 69.64%) as a yellow oil: ¹H NMR (400 MHz, Methanol-d4) δ 7.00-6.85 (m, 2H), 4.93 (ddd, J=2.0, 3.2, 6.2 Hz, 0.5H), 4.77 (ddd, J=2.1, 3.3, 6.1 Hz, 0.5H), 4.26 (q, J=7.1 Hz, 2H), 2.01-1.92 (m, 1H), 1.45-1.35 (m, 1H), 1.34-1.30 (m, 3H), 1.30-1.22 (m, 1H).

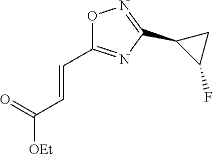 [SMILES: CCOC(=O)/C=C/c1nc([C@H]2C[C@@H]2F)no1]

### Step 5: (E)-ethyl 3-(3-((1R,2S)-2-fluorocyclopropyl)-1,2,4-oxadiazol-5-yl)acrylate

The cyclization reaction was prepared in a similar fashion to Method #4, Step 4.

The resulting residue was purified by column chromatography (silica gel, 100-200 mesh, 30-50% ethyl acetate in petroleum ether) affording ethyl (E)-3-[3-[(1R,2S)-2-fluorocyclopropyl]-1,2,4-oxadiazol-5-yl]prop-2-enoate (1.5 g, 80.97%) as a yellow oil: ¹H NMR (400 MHz, Methanol-d4) δ 7.44 (d, J=16.0 Hz, 1H), 7.00 (d, J=16.1 Hz, 1H), 5.08-4.88 (m, 1H), 4.30 (q, J=7.1 Hz, 2H), 2.71-2.55 (m, 1H), 1.79-1.62 (m, 1H), 1.44-1.36 (m, 1H), 1.36-1.32 (m, 3H). LCMS Rt=0.768 min, m/z=226.1 [M+H]⁺.

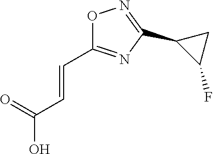 [SMILES: O=C(O)/C=C/c1nc([C@H]2C[C@@H]2F)no1]

### Step 6: (E)-3-(3-((1R,2S)-2-fluorocyclopropyl)-1,2,4-oxadiazol-5-yl)acrylic acid

The hydrolysis reaction was prepared in a similar fashion to Method #1, Step 3.

The reaction mixture was concentrated in vacuo affording (E)-3-[3-[(1R,2S)-2-fluorocyclopropyl]-1,2,4-oxadiazol-5-yl]prop-2-enoic acid (1.2 g, 91.33%) as a white solid, used in next step without any further purification: ¹H NMR (400 MHz, Chloroform-d) δ=7.52 (d, J=16.0 Hz, 1H), 7.00 (d, J=16.0 Hz, 1H), 5.08-4.81 (m, 1H), 2.63 (dddd, J=1.9, 7.0, 11.1, 17.0 Hz, 1H), 1.77-1.62 (m, 1H), 1.41 (qd, J=6.8, 12.1 Hz, 1H). LCMS Rt=0.572 min, m/z=198.0 [M+H]⁺.

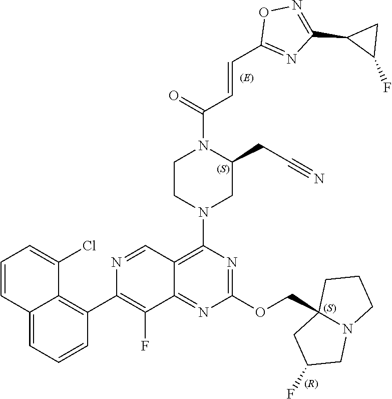

### Step 7: 2-((S)-4-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(3-((1R,2S)-2-fluorocyclopropyl)-1,2,4-oxadiazol-5-yl)acryloyl)piperazin-2-yl)acetonitrile

The amide coupling reaction was prepared in a similar fashion to Method #1, Step 4.

The crude product was purified by reverse phase prep-HPLC (column: Waters Xbridge BEH C18 100*30 mm*10 um; mobile phase: [water (NH₄HCO₃)-ACN]; B %: 40%-70%,10 min) affording 2-((S)-4-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(3-((1R,2S)-2-fluorocyclopropyl)-1,2,4-oxadiazol-5-yl)acryloyl)piperazin-2-yl)acetonitrile (19.25 mg, 26.62%) as a yellow solid. ¹H NMR (400 MHz, Acetonitrile-d3) δ 9.16-9.09 (m, 1H), 8.17-8.10 (m, 1H), 8.06-8.00 (m, 1H), 7.71-7.67 (m, 1H), 7.67-7.60 (m, 2H), 7.60-7.49 (m, 2H), 7.40-7.30 (m, 1H), 5.35-5.17 (m, 1H), 5.10-4.99 (m, 1H), 4.93-4.76 (m, 1H), 4.56-4.41 (m, 2H), 4.25-4.18 (m, 1H), 4.14-4.09 (m, 1H), 3.88-3.80 (m, 1H), 3.73-3.54 (m, 1H), 3.20-3.02 (m, 4H), 2.99-2.82 (m, 3H), 2.69-2.58 (m, 1H), 2.11-2.02 (m, 3H), 1.87 (br dd, J=6.5, 10.5 Hz, 3H), 1.82-1.62 (m, 2H), 1.34 (qd, J=6.6, 12.8 Hz, 1H). LCMS Rt=2.397 min, m/z=769.3 [M+H]⁺.

LCMS (5 to 95% acetonitrile in water+0.1% trifluoroacetic acid over 6 mins) retention time 2.397 min, ESI+found [M+H]=769.3.

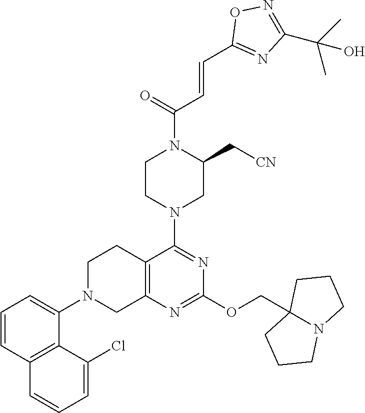 [SMILES: CC(C)(O)c1noc(/C=C/C(=O)N2CCN(c3nc(OCC45CCCN4CCC5)nc4c3CCN(c3cccc5cccc(Cl)c35)C4)C[C@@H]2CC#N)n1]

### Example 16 (Method 11): (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(3-(3-(2-hydroxypropan-2-yl)-1,2,4-oxadiazol-5-yl)acryloyl)piperazin-2-yl)acetonitrile

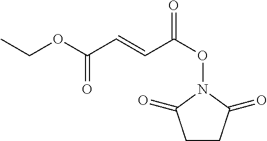 [SMILES: CCOC(=O)/C=C/C(=O)ON1C(=O)CCC1=O]

### Step 1: 2,5-dioxopyrrolidin-1-yl ethyl fumarate

The active ester formation was prepared in a similar fashion to Method #4, Step 1. The crude product was diluted with ethyl acetate (50 mL) and the resulting precipitate was filtered affording 2,5-dioxopyrrolidin-1-yl ethyl fumarate (30 g, 89.98%) as a yellow solid: ¹H NMR (400 MHz, Chloroform-d) δ 7.13-7.00 (m, 2H), 4.30 (q, J=7.3 Hz, 2H), 2.90-2.86 (m, 4H), 1.34 (t, J=7.1 Hz, 3H). LCMS Rt=0.454 min, m/z=241.1 [M+H]⁺.

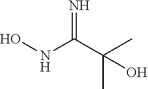

### Step 2: N,2-dihydroxy-2-methylpropanimidamide

The addition reaction was prepared in a similar fashion to Method #4, Step 2. The resulting residue was purified by column chromatography (silica gel, 100-200 mesh, 0-100% ethyl acetate in petroleum ether) affording N,2-dihydroxy-2-methylpropanimidamide (2.2 g) as a white solid. LCMS Rt=0.351 min, m/z=118.1 [M+H]⁺.

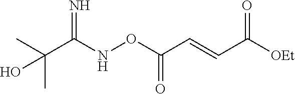 [SMILES: CCOC(=O)/C=C/C(=O)ONC(=N)C(C)(C)O]

### Step 3: (E)-ethyl 4-((2-hydroxy-2-methylpropanimidamido)oxy)-4-oxobut-2-enoate

The amide coupling reaction was prepared in a similar fashion to Method #4, Step 3. The resulting residue was purified by column chromatography (silica gel, 100-200 mesh, 0-100% ethyl acetate in petroleum ether) affording (E)-ethyl 4-((2-hydroxy-2-methylpropanimidamido)oxy)-4-oxobut-2-enoate (2.2 g, 49.89%) as a white solid. LCMS Rt=0.552 min, m/z=244.1 [M+H]+.

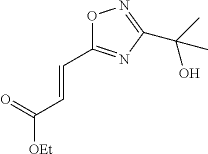 [SMILES: CCOC(=O)/C=C/c1nc(C(C)(C)O)no1]

### Step 4: (E)-ethyl 3-(3-(2-hydroxypropan-2-yl)-1,2,4-oxadiazol-5-yl)acrylate

The cyclization reaction was prepared in a similar fashion to Method #4, Step 4. The resulting residue was purified by column chromatography (silica gel, 100-200 mesh, 0-100% ethyl acetate in petroleum ether) affording (E)-ethyl 3-(3-(2-hydroxypropan-2-yl)-1,2,4-oxadiazol-5-yl)acrylate (350 mg) as a white solid: ¹H NMR (400 MHz, Chloroform-d) δ 7.41 (d, J=16.0 Hz, 1H), 6.97 (d, J=16.0 Hz, 1H), 4.31-4.17 (m, 2H), 1.64-1.55 (m, 6H), 1.34-1.23 (m, 3H). LCMS Rt=0.590 min, m/z=226.1 [M+H]⁺.

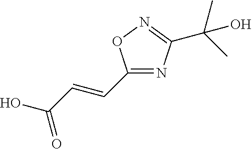

### Step 5: (E)-3-(3-(2-hydroxypropan-2-yl)-1,2,4-oxadiazol-5-yl)acrylic acid

The hydrolysis reaction was prepared in a similar fashion to Method #1, Step 3. The crude product was concentrated in vacuo affording (E)-3-(3-(2-hydroxypropan-2-yl)-1,2,4-oxadiazol-5-yl)acrylic acid (180 mg, 55.94%) as a white solid used in the next step without further purification. LCMS Rt=0.467 min, m/z=198.1 [M+H]⁺.

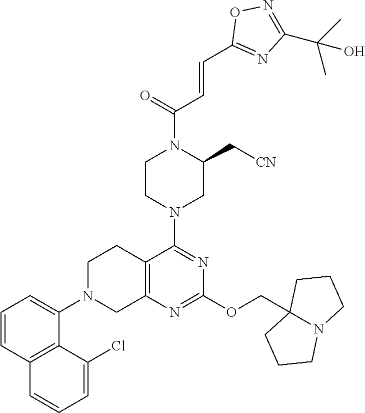 [SMILES: CC(C)(O)c1noc(/C=C/C(=O)N2CCN(c3nc(OCC45CCCN4CCC5)nc4c3CCN(c3cccc5cccc(Cl)c35)C4)C[C@@H]2CC#N)n1]

### Step 6: (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(3-(3-(2-hydroxypropan-2-yl)-1,2,4-oxadiazol-5-yl)acryloyl)piperazin-2-yl)acetonitrile

The amide coupling reaction was prepared in a similar fashion to Method #1, Step 4. The crude product was purified by reverse phase HPLC (column: Waters Xbridge BEH C18 100*30 mm*10 um; mobile phase: [water (ammonium bicarbonate)-acetonitrile]; B %: 35%-65%,8 min) affording (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(3-(3-(2-hydroxypropan-2-yl)-1,2,4-oxadiazol-5-yl)acryloyl)piperazin-2-yl)acetonitrile (9.8 mg, 10.74%) as a yellow solid: ¹H NMR (400 MHz, Acetonitrile-d3) δ 7.86 (d, J=8.3 Hz, 1H), 7.75-7.59 (m, 2H), 7.58-7.47 (m, 2H), 7.44-7.29 (m, 3H), 5.20-4.54 (m, 1H), 4.26 (dd, J=8.3, 17.5 Hz, 1H), 4.14-4.00 (m, 2H), 4.00-3.96 (m, 2H), 3.82-3.69 (m, 1H), 3.61-3.49 (m, 2H), 3.18 (br d, J=2.8 Hz, 2H), 3.16-3.02 (m, 3H), 2.99-2.85 (m, 3H), 2.81 (br d, J=7.0 Hz, 1H), 2.68-2.51 (m, 3H), 2.11-2.06 (m, 2H), 1.91-1.73 (m, 6H), 1.59-1.57 (m, 6H). LCMS Rt=2.287 min, m/z=737.3 [M+H]⁺.

LCMS (5 to 95% acetonitrile in water+0.03% ammonium bicarbonate over 6 mins) retention time 2.287 min, ESI+found [M+H]=737.3.

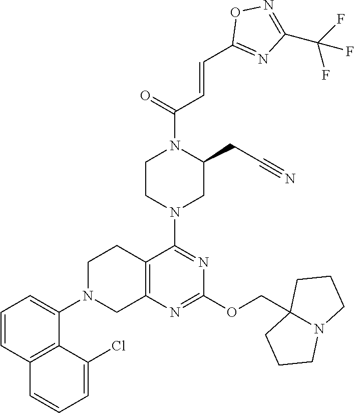 [SMILES: N#CC[C@H]1CN(c2nc(OCC34CCCN3CCC4)nc3c2CCN(c2cccc4cccc(Cl)c24)C3)CCN1C(=O)/C=C/c1nc(C(F)(F)F)no1]

### Example 17 (Method 12): (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(3-(3-(trifluoromethyl)-1,2,4-oxadiazol-5-yl)acryloyl)piperazin-2-yl)acetonitrile

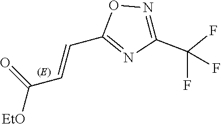 [SMILES: CCOC(=O)/C=C/c1nc(C(F)(F)F)no1]

### Step 1: (E)-ethyl 3-(3-(trifluoromethyl)-1,2,4-oxadiazol-5-yl)acrylate

The cyclization reaction was prepared in a similar fashion to Method #1, Step 2. The resulting residue was purified by column chromatography (silica gel, 100-200 mesh, 50-100% ethyl acetate in petroleum ether) affording (E)-ethyl 3-(3-(trifluoromethyl)-1,2,4-oxadiazol-5-yl)acrylate (700 mg, 37.96%) as a yellow oil: ¹H NMR (400 MHz, Chloroform-d) δ 7.46 (d, J=16.1 Hz, 1H), 7.09 (d, J=16.1 Hz, 1H), 4.27 (q, J=7.1 Hz, 2H), 1.29 (t, J=7.1 Hz, 3H)

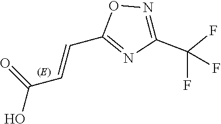 [SMILES: O=C(O)/C=C/c1nc(C(F)(F)F)no1]

### Step 2: (E)-3-(3-(trifluoromethyl)-1,2,4-oxadiazol-5-yl)acrylic acid

The hydrolysis reaction was prepared in a similar fashion to Method #1, Step 3. The reaction mixture was quenched with hydrochloric acid (1M, 1 mL) and adjusted pH to 2, then extracted with dichloromethane (3×10 mL). The combined organic layers were dried over sodium sulphate and concentrated in vacuo affording (E)-3-(3-(trifluoromethyl)-1,2,4-oxadiazol-5-yl)acrylic acid (500 mg, crude) as a yellow oil used in next step without any further purification. LCMS Rt=0.449 min, m/z=208.0 [M+H]⁺.

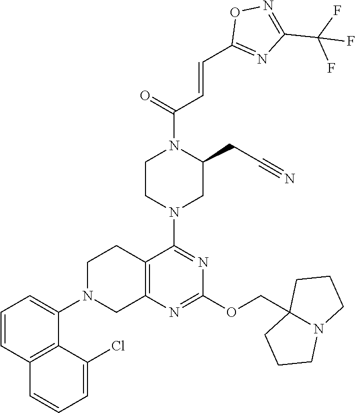 [SMILES: N#CC[C@H]1CN(c2nc(OCC34CCCN3CCC4)nc3c2CCN(c2cccc4cccc(Cl)c24)C3)CCN1C(=O)/C=C/c1nc(C(F)(F)F)no1]

### Step 3: (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(3-(3-(trifluoromethyl)-1,2,4-oxadiazol-5-yl)acryloyl)piperazin-2-yl)acetonitrile

The amide coupling reaction was prepared in a similar fashion to Method #1, Step 4. The crude product was purified by reverse phase HPLC (column: Waters Xbridge BEH C18 100*30 mm*10 um; mobile phase: [water (NH₄HCO₃)-ACN]; B %: 50%-80%, 10 min) affording (S,E)-2-(4-(7-(8-chloronaphthalen-1-yl)-2-((hexahydro-1H-pyrrolizin-7a-yl)methoxy)-5,6,7,8-tetrahydropyrido[3,4-d]pyrimidin-4-yl)-1-(3-(3-(trifluoromethyl)-1,2,4-oxadiazol-5-yl)acryloyl)piperazin-2-yl)acetonitrile (19.97 mg, 14.71%) as a yellow amorphous solid: ¹H NMR (400 MHz, Acetonitrile-d3) δ 7.96-7.82 (m, 2H), 7.78-7.70 (m, 1H), 7.63-7.50 (m, 3H), 7.49-7.35 (m, 2H), 5.21-5.05 (m, 1H), 4.81-4.70 (m, 0.5H), 4.62-4.54 (m, 0.5H), 4.30 (br dd, J=6.7, 17.1 Hz, 1H), 4.16-4.03 (m, 4H), 3.92-3.70 (m, 2H), 3.64-3.54 (m, 1H), 3.45-3.33 (m, 1H), 3.21-3.01 (m, 6H), 2.93-2.82 (m, 1H), 2.73-2.60 (m, 3H), 2.34-2.24 (m, 2H), 1.95-1.78 (m, 4H), 1.76-1.62 (m, 2H). LCMS Rt=3.275 min, m/z=747.3 [M+H]⁺. LCMS (5 to 95% acetonitrile in water+0.03% ammonium bicarbonate over 6 mins) retention time 3.275 min, ESI+found [M+H]=747.3.

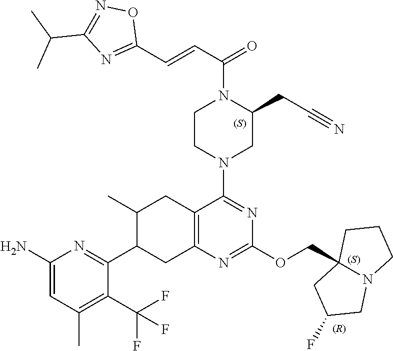

### Example 66: 2-((2S)-4-(7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)-1-((E)-3-(3-isopropyl-1,2,4-oxadiazol-5-yl)acryloyl)piperazin-2-yl)acetonitrile

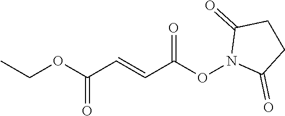 [SMILES: CCOC(=O)/C=C/C(=O)ON1C(=O)CCC1=O]

2,5-dioxopyrrolidin-1-yl ethyl fumarate

To a solution of (E)-4-ethoxy-4-oxobut-2-enoic acid (30 g, 208.15 mmol) and 1-hydroxypyrrolidine-2,5-dione (71.87 g, 624.46 mmol) in acetonitrile (150 mL) was added 1-(3-dimethylaminopropyl)-3-ethylcarbodiimide (79.81 g, 416.31 mmol), and the mixture was stirred at 25° C. for 2 h. The mixture was diluted with water (150 mL) and extracted with ethyl acetate (3×100 mL). The combined organic layers were dried over sodium sulphate and concentrated in vacuo. The crude residue was diluted with ethyl acetate (50 mL), and the resulting precipitate was filtered affording 2,5-dioxopyrrolidin-1-yl ethyl fumarate (45 g, 86.05%) as a yellow oil: ¹H NMR (400 MHz, Chloroform -d) δ 7.15-6.92 (m, 2H), 4.29 (q, J=7.1 Hz, 2H), 2.86 (s, 4H), 1.32 (t, J=7.2 Hz, 3H). LCMS Rt=0.459 min, m/z=242.1 [M+H]⁺.

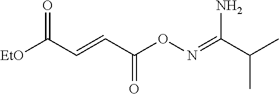 [SMILES: CCOC(=O)/C=C/C(=O)O/N=C(\N)C(C)C]

### Step 1: (E)-ethyl 4-(((Z)-(1-amino-2-methylpropylidene)amino)oxy)-4-oxobut-2-enoate

The amide coupling reaction was prepared in a similar fashion to Method #4, Step 3.

The reaction mixture was concentrated in vacuo affording (E)-ethyl 4-(((Z)-(1-amino-2-methylpropylidene)amino)oxy)-4-oxobut-2-enoate (15 g, crude), which was used in the next step without further purification. LCMS Rt=0.545 min, m/z=229.1 [M+H]⁺.

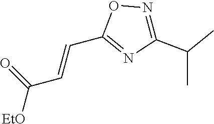

### Step 2: (E)-ethyl 3-(3-isopropyl-1,2,4-oxadiazol-5-yl)acrylate

The cyclization reaction was prepared in a similar fashion to Method #4, Step 4. The reaction mixture was concentrated in vacuo affording (E)-ethyl 3-(3-isopropyl-1,2,4-oxadiazol-5-yl)acrylate (15 g, crude), which was used in the next step without further purification. LCMS Rt=0.740 min, m/z=211.1 [M+H]⁺.

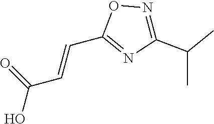

### Step 4: (E)-3-(3-isopropyl-1,2,4-oxadiazol-5-yl)acrylic acid

The hydrolysis reaction was prepared in a similar fashion to Method #1, Step 3. The reaction mixture was concentrated in vacuo affording (E)-3-(3-isopropyl-1,2,4-oxadiazol-5-yl)prop-2-enoic acid (2 g, crude), which was used in the next step without further purification. LCMS Rt=0.638 min, m/z=183.1 [M+H]⁺.

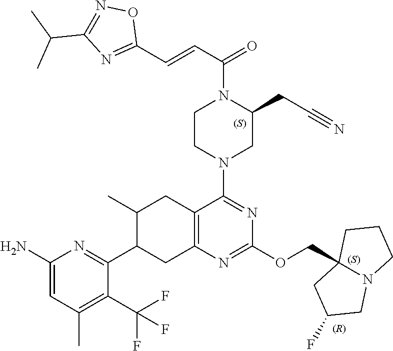

### Step 5: 2-((2S)-4-(7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)-1-((E)-3-(3-isopropyl-1,2,4-oxadiazol-5-yl)acryloyl)piperazin-2-yl)acetonitrile

To a solution of 2-((2S)-4-(7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)piperazin-2-yl)acetonitrile (50 mg, 69.77 umol, trifluoroacetic salt) and (E)-3-(3-isopropyl-1,2,4-oxadiazol-5-yl)prop-2-enoic acid (10.17 mg, 55.81 umol) in dichloromethane (2 mL) was added N,N-diisopropylethylamine (45.08 mg, 348.83 umol) and 2,4,6-tripropyl-1,3,5,2,4,6trioxatriphosphinane 2,4,6-trioxide (88.79 mg, 139.53 umol, 50% purity in ethyl acetate) at −10° C. The mixture was stirred at -10 C for 0.5 h. The reaction mixture was concentrated in vacuo and purified by reverse phase HPLC (C18 150*40 mm*10 um; mobile phase: [water (NH₄HCO₃)-ACN]; B %: 50%-80%, 8 min) affording 2-((2S)-4-(7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)-1-((E)-3-(3-isopropyl-1,2,4-oxadiazol-5-yl)acryloyl)piperazin-2-yl)acetonitrile (14.17 mg, 25.66% yield) as a yellow oil: ¹H NMR (400 MHz, Acetonitrile-d3) δ 7.78-7.56 (m, 1H), 7.45-7.31 (m, 1H), 6.28 (s, 1H), 5.35-5.05 (m, 4H), 4.78-4.48 (m, 1H), 4.10-3.83 (m, 5H), 3.31 (br dd, J=3.7, 13.8 Hz, 1H), 3.18-3.11 (m, 4H), 3.08-3.01 (m, 3H), 2.94-2.82 (m, 3H), 2.79-2.57 (m, 2H), 2.48-2.32 (m, 5H), 2.15 (br s, 1H), 2.08 (br d, J=1.9 Hz, 1H), 2.05-2.00 (m, 1H), 1.92-1.87 (m, 1H), 1.86-1.79 (m, 2H), 1.35 (d, J=6.9 Hz, 6H), 0.80 (br d, J=6.3 Hz, 3H).

LCMS (5% to 95% acetonitrile in water+0.03% ammonium bicarbonate over 6 min); retention time 3.090 min, ESI+found [M+H]=767.4.

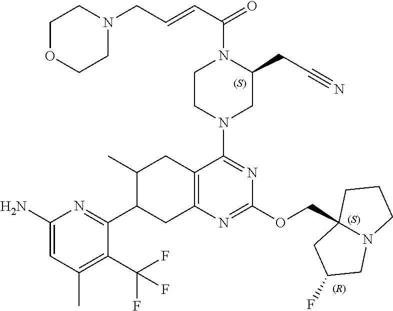

### Example 67: 2-((2S)-4-(7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)-1-((E)-4-morpholinobut-2-enoyl)piperazin-2-yl)acetonitrile

To a solution of diethyl (2-((2S)-4-(7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)-2-(cyanomethyl)piperazin-1-yl)-2-oxoethyl)phosphonate (40 mg, 44.70 umol, trifluoroacetate salt) and 2-morpholinoacetaldehyde (11.55 mg, 89.40 umol) in acetonitrile (1 mL) was added lithium chloride (3.79 mg, 89.40 umol) and N,N-diisopropylethylamine (17.33 mg, 134.11 umol). The mixture was stirred at 20° C. for 1 h. The reaction mixture was concentrated in vacuo and purified by reverse phase HPLC (column: Waters Xbridge Prep OBD C18 150*40 mm*10 um; mobile phase: [water (NH₄HCO₃)-ACN]; B %: 30%-60%, 8 min) affording 2-((2S)-4-(7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)-1-((E)-4-morpholinobut-2-enoyl)piperazin-2-yl)acetonitrile (3.66 mg, 9.57% yield) as a yellow oil: ¹H NMR (400 MHz, Acetonitrile-d3) δ 6.78-6.59 (m, 1H), 6.59-6.38 (m, 1H), 6.17 (s, 1H), 5.25-4.83 (m, 4H), 3.93 (dd, J=1.3, 10.4 Hz, 1H), 3.89-3.63 (m, 3H), 3.58-3.37 (m, 5H), 3.16-3.00 (m, 6H), 3.00-2.88 (m, 4H), 2.82-2.71 (m, 3H), 2.67-2.45 (m, 3H), 2.33 (br s, 4H), 2.28-2.24 (m, 3H), 1.96 (br s, 1H), 1.91 (br d, J=4.6 Hz, 1H), 1.82-1.75 (m, 2H), 1.73-1.65 (m, 2H), 0.68 (dd, J=2.0, 6.4 Hz, 3H).

LCMS (5% to 95% acetonitrile in water+0.03% ammonium bicarbonate over 6 min); retention time 2.712 min, ESI+found [M+H]=756.4.

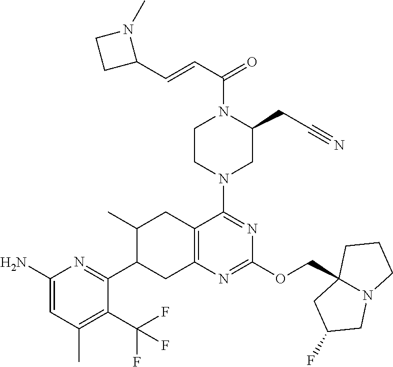 [SMILES: Cc1cc(N)nc(C2Cc3nc(OC[C@@]45CCCN4C[C@H](F)C5)nc(N4CCN(C(=O)/C=C/C5CCN5C)[C@@H](CC#N)C4)c3CC2C)c1C(F)(F)F]

### Example 68: 2-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-1-((E)-3-(1-methylazetidin-2-yl)acryloyl)piperazin-2-yl)acetonitrile

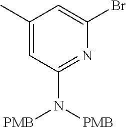

### Step 1: 6-bromo-N,N-bis(4-methoxybenzyl)-4-methylpyridin-2-amine

To a solution of sodium hydride (128.30 g, 2.67 mol, 60% purity) in N,N-dimethylformamide (2000 mL) was added 6-bromo-4-methyl-pyridin-2-amine (100 g, 534.65 mmol) at 0° C. The mixture was stirred at 0° C. for 1 h, and 1-(chloromethyl)-4-methoxy-benzene (209.33 g, 1.34 mol) was added. The resulting mixture was stirred at 25° C. for 11 h. The reaction mixture was quenched with a solution of saturated ammonium chloride (500 mL) at 0° C. and extracted with ethyl acetate (3×500 mL). The combined organic layers were dried over sodium sulphate and concentrated in vacuo. The crude product was triturated with water (50 mL), filtered, and the filter cake was collected and dried in vacuo affording 6-bromo-N,N-bis(4-methoxybenzyl)-4-methylpyridin-2-amine (220 g, 96.29%) as a yellow solid: ¹H NMR (400 MHz, Chloroform-d) δ 7.07 (d, J=8.6 Hz, 4H), 6.80-6.73 (m, 4H), 6.51 (s, 1H), 6.07 (s, 1H), 4.55 (s, 4H), 3.71 (s, 6H), 2.04 (s, 3H). LCMS Rt=2.438 min, m/z=427.1 [M+H]⁺.

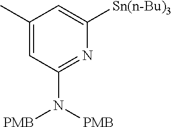

### Step 2: N,N-bis(4-methoxybenzyl)-4-methyl-6-(tributylstannyl)pyridin-2-amine

To a solution of 6-bromo-N,N-bis[(4-methoxyphenyl)methyl]-4-methyl-pyridin-2-amine (50 g, 117 mmol) in dioxane (4.0 L) was added lithium chloride (24.8 g, 582.02 mmol), tricyclohexylphosphane (6.56 g, 23.4 mmol), tris(dibenzylideneacetone)dipalladium(0) (10.72 g, 11.7 mmol) and tributyl(tributylstannyl)stannane (169.68 g, 292.52 mmol). The mixture was stirred at 110° C. for 12 h under a nitrogen atmosphere. The reaction mixture was filtered, and the filtrate was concentrated to dryness in vacuo. The residue was purified by column chromatography (silica gel, 100-200 mesh, 0-10% ethyl acetate in petroleum ether) affording N,N-bis(4-methoxybenzyl)-4-methyl-6-(tributylstannyl)pyridin-2-amine (74 g, 99.21%) as a yellow oil. LCMS Rt=2.407 min, m/z=639.3 [M+H]⁺.

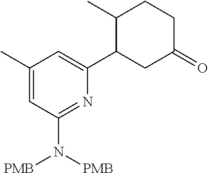

### Step 3: 3-(6-(bis(4-methoxybenzyl)amino)-4-methylpyridin-2-yl)-4-methylcyclohexanone

To a solution of N,N-bis[(4-methoxyphenyl)methyl]-4-methyl-6-tributylstannyl-pyridin-2-amine (38 g, 59.61 mmol) in tetrahydrofuran (2000 mL) was added chloro(1,5-cyclooctadiene)rhodium(I) dimer (2.94 g, 5.96 mmol, 0.1 eq), water (107.39 mg, 5.96 mmol), and 4-methylcyclohex-2-en-1-one (7.88 g, 71.53 mmol). The mixture was stirred at 60° C. for 12 h under a nitrogen atmosphere. The reaction mixture was concentrated to dryness in vacuo. The residue was purified by column chromatography (silica gel, 100-200 mesh, 0-10% ethyl acetate in petroleum ether) affording 3-(6-(bis(4-methoxybenzyl)amino)-4-methylpyridin-2-yl)-4-methylcyclohexanone (35 g, 64.02%) as a yellow oil. LCMS Rt=1.824 min, m/z=459.3 [M+H]⁺.

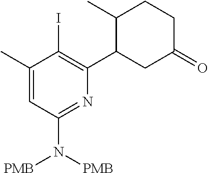

### Step 4: 3-(6-(bis(4-methoxybenzyl)amino)-3-iodo-4-methylpyridin-2-yl)-4-methylcyclohexanone

To a solution of 3-(6-(bis(4-methoxybenzyl)amino)-4-methylpyridin-2-yl)-4-methylcyclohexanone (34 g, 74.14 mmol) in N,N-dimethylformamide (500 mL) was added N-iodosuccinimide (33.36 g, 148.28 mmol). The mixture was stirred at 25° C. for 3 h. The reaction mixture was concentrated to dryness in vacuo. The remaining residue was purified by column chromatography (silica gel, 100-200 mesh, 0-10% ethyl acetate in petroleum ether) affording 3-(6-(bis(4-methoxybenzyl)amino)-3-iodo-4-methylpyridin-2-yl)-4-methylcyclohexanone (19 g, 43.85%) as a yellow oil. LCMS Rt=1.092 min, m/z=585.2 [M+H]⁺.

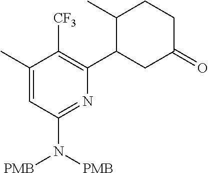

### Step 5: 3-(6-(bis(4-methoxybenzyl)amino)-4-methyl-3-(trifluoromethyl)pyridin-2-yl)-4-methylcyclohexanone

To a solution of 3-(6-(bis(4-methoxybenzyl)amino)-3-iodo-4-methylpyridin-2-yl)-4-methylcyclohexanone (15 g, 25.66 mmol) in N,N-dimethylformamide (300 mL) was added cuprous iodide (14.66 g, 76.99 mmol) and methyl 2,2-difluoro-2-fluorosulfonyl-acetate (24.65 g, 128.32 mmol). The mixture was stirred at 90° C. for 2 h under a nitrogen atmosphere. The reaction mixture was filtered, and the filtrate was concentrated to dryness in vacuo. The remaining residue was purified by column chromatography (silica gel, 100-200 mesh, 0-10% ethyl acetate in petroleum ether) affording 3-(6-(bis(4-methoxybenzyl)amino)-4-methyl-3-(trifluoromethyl)pyridin-2-yl)-4-methylcyclohexanone (12 g, 88.80%) as a yellow oil. LCMS Rt=0.875 min, m/z=527.2 [M+H]⁺.

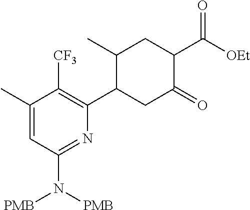

### Step 6: ethyl 4-(6-(bis(4-methoxybenzyl)amino)-4-methyl-3-(trifluoromethyl)pyridin-2-yl)-5-methyl-2-oxocyclohexanecarboxylate

To a solution of 3-(6-(bis(4-methoxybenzyl)amino)-4-methyl-3-(trifluoromethyl)pyridin-2-yl)-4-methylcyclohexanone (12 g, 22.78 mmol) in tetrahydrofuran (600 mL) was added lithium hexamethyldisilazide (1 M, 34.18 mL) at −78° C. under a nitrogen atmosphere. The mixture was stirred at −78° C. for 1 h, and ethyl cyanoformate (2.7 g, 27.34 mmol) was added. The resulting mixture was stirred at −78° C. for 0.5 h under a nitrogen atmosphere. The reaction mixture was quenched with a saturated solution of ammonium chloride (100 mL) and extracted with ethyl acetate (3×150 mL). The combined organic layers were dried over sodium sulphate and concentrated in vacuo affording ethyl 4-[6-[bis[(4-methoxyphenyl)methyl]amino]-4-methyl-3-(trifluoromethyl)-2-pyridyl]-5-methyl-2-oxo-cyclohexanecarboxylate (13.6 g, crude) as a yellow oil, which was used in the next step without further purification. LCMS Rt=1.235 min, m/z=599.3 [M+H]⁺.

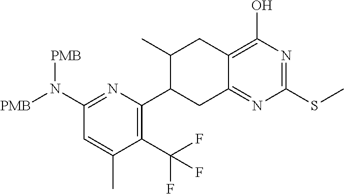

### Step 7: 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-ol

To a solution of ethyl 4-(6-(bis(4-methoxybenzyl)amino)-4-methyl-3-(trifluoromethyl)pyridin-2-yl)-5-methyl-2-oxocyclohexanecarboxylate (13 g, 21.72 mmol) in a mixture of ethanol (1200 mL) and water (240 mL) was added sodium bicarbonate (45.61 g, 542.89 mmol) and S-methylisothiourea hemisulfate (60.45 g, 217.15 mmol). The mixture was stirred at 50° C. for 12 h. The reaction mixture was filtered, and the filtrate was concentrated to dryness in vacuo. The remaining residue was purified by column chromatography (silica gel, 100-200 mesh, 0-10% ethyl acetate in petroleum ether) affording 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-ol (4.7 g, 34.65%) as a yellow solid. LCMS Rt=1.046 min, m/z=625.2 [M+H]⁺.

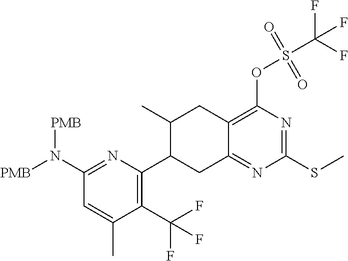

### Step 8: 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl trifluoromethanesulfonate

To a solution of 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-ol (1.5 g, 2.40 mmol) in dichloromethane (100 mL) was added triethylamine (970 mg, 9.60 mmol) and trifluoromethanesulfonic anhydride (2.37 g, 8.40 mmol) at 0° C. under a nitrogen atmosphere. The mixture was stirred at 25° C. for 1 h. The reaction mixture was quenched with a saturated solution of sodium bicarbonate (30 mL) at 0° C. and extracted with dichloromethane (3×100 mL). The combined organic layers were dried over sodium sulphate and concentrated in vacuo affording 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl trifluoromethanesulfonate (1.8 g, crude) as a yellow oil, which was used in the next step without further purification. LCMS Rt=3.664 min, m/z=757.2 [M+H]⁺.

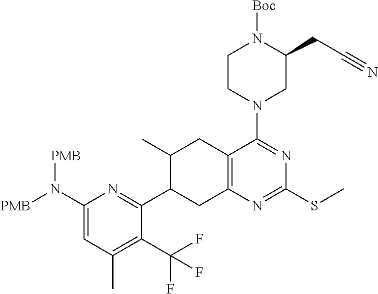

### Step 9: (2S)-tert-butyl 4-(7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl)-2-(cyanomethyl)piperazine-1-carboxylate

To a solution of tert-butyl (2S)-2-(cyanomethyl)piperazine-1-carboxylate (1.21 g, 5.35 mmol) in N,N-dimethylformamide (20 mL) was added N,N-diisopropylethylamine (1.84 g, 14.27 mmol) and 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl trifluoromethanesulfonate (2.7 g, 3.57 mmol) at 0° C. The mixture was stirred at 20° C. for 12 h. The reaction mixture was concentrated in vacuo and purified by column chromatography (silica gel, 100-200 mesh, 0-50% ethyl acetate in petroleum ether) affording (2S)-tert-butyl 4-(7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl)-2-(cyanomethyl)piperazine-1-carboxylate (2.6 g, 87.59%) as a yellow oil. LCMS Rt=0.752 min, m/z=832.4 [M+H]⁺.

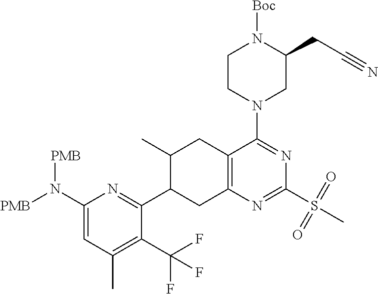

### Step 10: (2S)-tert-butyl 4-(7-(6-(bis(4-methoxybenzyl)amino)-4-methyl-3-(trifluoromethyl)pyridin-2-yl)-6-methyl-2-(methylsulfonyl)-5,6,7,8-tetrahydroquinazolin-4-yl)-2-(cyanomethyl)piperazine-1-carboxylate

To a solution of (2S)-tert-butyl 4-(7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl)-2-(cyanomethyl)piperazine-1-carboxylate (2.5 g, 3.00 mmol) in dichloromethane (300 mL) was added meta-chloroperoxybenzoic acid (1.94 g, 9.01 mmol, 85% purity) at 0° C., and the mixture was stirred for 1 h. The reaction mixture was quenched with saturated sodium sulfite (30 mL) and extracted with dichloromethane (3×50 mL). The combined organic layers were dried over sodium sulphate and concentrated in vacuo. The crude residue was purified by column chromatography (silica gel, 100-200 mesh, 0-50% ethyl acetate in petroleum ether) affording (2S)-tert-butyl 4-(7-(6-(bis(4-methoxybenzyl)amino)-4-methyl-3-(trifluoromethyl)pyridin-2-yl)-6-methyl-2-(methylsulfonyl)-5,6,7,8-tetrahydroquinazolin-4-yl)-2-(cyanomethyl)piperazine-1-carboxylate (1.6 g, 61.63%) as a yellow oil. LCMS Rt=1.027 min, m/z=864.4 [M+H]⁺.

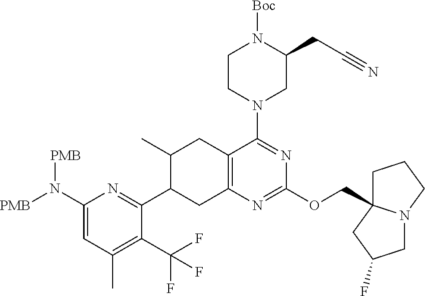

### Step 11: (2S)-tert-butyl 4-(7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-2-(cyanomethyl)piperazine-1-carboxylate

To a solution of (2S)-tert-butyl 4-(7-(6-(bis(4-methoxybenzyl)amino)-4-methyl-3-(trifluoromethyl)pyridin-2-yl)-6-methyl-2-(methylsulfonyl)-5,6,7,8-tetrahydroquinazolin-4-yl)-2-(cyanomethyl)piperazine-1-carboxylate (1.6 g, 1.85 mmol) and ((2R,7aS)-2-fluorohexahydro-1H-pyrrolizin-7a-yl)methanol (442.23 mg, 2.78 mmol) in toluene (40 mL) was added sodium tert-butoxide (533.92 mg, 5.56 mmol) at −30° C., and the mixture was stirred for 10 min. The reaction mixture was quenched with saturated ammonium chloride (30 mL) at 0° C. and extracted with dichloromethane (3×100 mL). The combined organic layers were dried over sodium sulphate and concentrated in vacuo. The remaining residue was purified by column chromatography (silica gel, 100-200 mesh, 0-100% ethyl acetate in petroleum ether) affording (2S)-tert-butyl 4-(7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-2-(cyanomethyl)piperazine-1-carboxylate (1.6 g, 91.61%) as a yellow solid. LCMS Rt=0.960 min, m/z=943.5 [M+H]⁺.

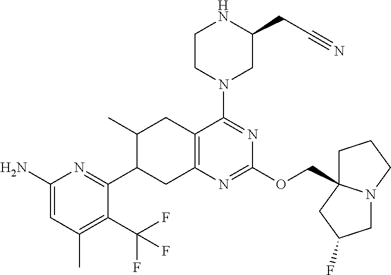 [SMILES: Cc1cc(N)nc(C2Cc3nc(OC[C@@]45CCCN4C[C@H](F)C5)nc(N4CCN[C@@H](CC#N)C4)c3CC2C)c1C(F)(F)F]

### Step 12: 2-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)piperazin-2-yl)acetonitrile

A mixture of (2S)-tert-butyl 4-(7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-2-(cyanomethyl)piperazine-1-carboxylate (800 mg, 848.28 umol) in trifluoroacetic acid (6 mL) was stirred at 80° C. for 0.5 h. The reaction mixture was concentrated in vacuo. The crude product was purified by reverse phase prep-HPLC (column: Phenomenex luna C18 250*50 mm*10 um; mobile phase: [water (TFA)-ACN]; B %: 20%-60%, 10 min) affording 2-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)piperazin-2-yl)acetonitrile (340 mg, 55.92%, trifluoroacetate salt) as a white solid. LCMS Rt=1.461 min, m/z=604.3 [M+H]⁺.

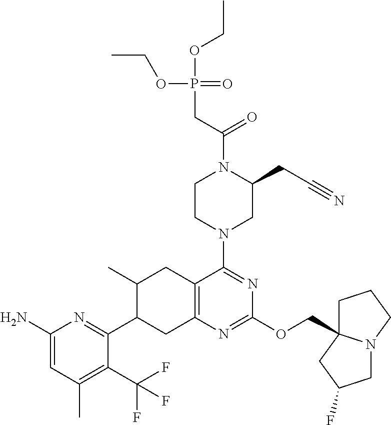 [SMILES: CCOP(=O)(CC(=O)N1CCN(c2nc(OC[C@@]34CCCN3C[C@H](F)C4)nc3c2CC(C)C(c2nc(N)cc(C)c2C(F)(F)F)C3)C[C@@H]1CC#N)OCC]

### Step 13: diethyl (2-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-2-(cyanomethyl)piperazin-1-yl)-2-oxoethyl)phosphonate

To a solution of 2-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)piperazin-2-yl)acetonitrile (133 mg, 185.58 umol, trifluoroacetate salt) and 2-diethoxyphosphorylacetic acid (25.48 mg, 129.90 umol) in dichloromethane (3 mL) was added N,N-diisopropylethylamine (119.92 mg, 927.88 umol) and 2,4,6-tripropyl-1,3,5,2,4,6-trioxatriphosphinane 2,4,6-trioxide (236.19 mg, 371.15 umol, 50% purity in ethyl acetate) at −10° C. The mixture was stirred at −10° C. for 0.5 h and then concentrated in vacuo. The crude product was purified by reverse phase prep-HPLC (column: Phenomenex Luna 80*30 mm*3 um; mobile phase: [water (TFA)-ACN]; B %: 15%-50%, 8 min) affording diethyl (2-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-2-(cyanomethyl)piperazin-1-yl)-2-oxoethyl)phosphonate (100 mg, 60.22%, trifluoroacetate salt) as a yellow oil. LCMS Rt=1.523 min, m/z=781.4 [M+H]⁺.

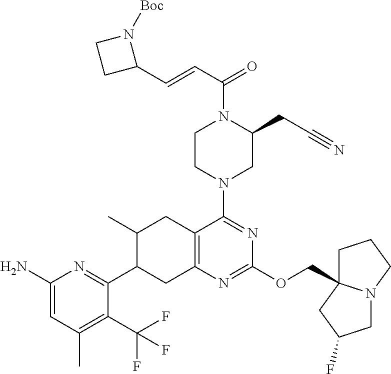 [SMILES: Cc1cc(N)nc(C2Cc3nc(OC[C@@]45CCCN4C[C@H](F)C5)nc(N4CCN(C(=O)/C=C/C5CCN5C(=O)OC(C)(C)C)[C@@H](CC#N)C4)c3CC2C)c1C(F)(F)F]

### Step 14: tert-butyl 2-((E)-3-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-2-(cyanomethyl)piperazin-1-yl)-3-oxoprop-1-en-1-yl)azetidine-1-carboxylate

To a solution of diethyl (2-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-2-(cyanomethyl)piperazin-1-yl)-2-oxoethyl)phosphonate (60 mg, 67.05 umol, trifluoroacetate salt) in acetonitrile (3 mL) was added N,N-diisopropylethylamine (26.00 mg, 201.16 umol), lithium chloride (5.69 mg, 134.11 umol) and tert-butyl 2-formylazetidine-1-carboxylate (24.84 mg, 134.11 umol). The mixture was stirred at 20° C. for 1 h and concentrated in vacuo affording tert-butyl 2-((E)-3-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-2-(cyanomethyl)piperazin-1-yl)-3-oxoprop-1-en-1-yl)azetidine-1-carboxylate (54 mg, crude) as a yellow oil, which was used in the next step without any further purification. LCMS Rt=0.618 min, m/z=812.4 [M+H]⁺.

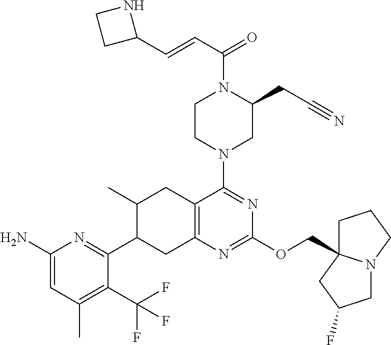 [SMILES: Cc1cc(N)nc(C2Cc3nc(OC[C@@]45CCCN4C[C@H](F)C5)nc(N4CCN(C(=O)/C=C/C5CCN5)[C@@H](CC#N)C4)c3CC2C)c1C(F)(F)F]

### Step 15: 2-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-1-((E)-3-(azetidin-2-yl)acryloyl)piperazin-2-yl)acetonitrile

A mixture of tert-butyl 2-((E)-3-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-2-(cyanomethyl)piperazin-1-yl)-3-oxoprop-1-en-1-yl)azetidine-1-carboxylate (40 mg, 49.27 umol) and trifluoroacetic acid (0.5 mL) in dichloromethane (1.5 mL) was stirred at 20° C. for 0.5 h. The reaction mixture was concentrated in vacuo affording 2-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-1-((E)-3-(azetidin-2-yl)acryloyl)piperazin-2-yl)acetonitrile (40 mg, crude, trifluoroacetate salt) as a yellow oil, which was used in the next step without any further purification. LCMS Rt=0.455 min, m/z=712.4 [M+H]⁺.

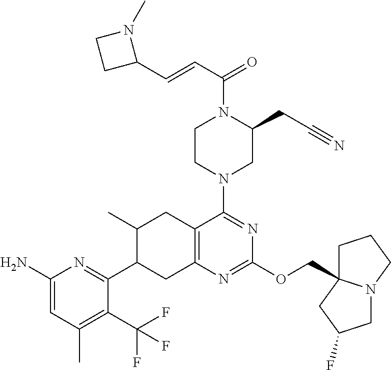 [SMILES: Cc1cc(N)nc(C2Cc3nc(OC[C@@]45CCCN4C[C@H](F)C5)nc(N4CCN(C(=O)/C=C/C5CCN5C)[C@@H](CC#N)C4)c3CC2C)c1C(F)(F)F]

### Step 16: 2-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-1-((E)-3-(1-methylazetidin-2-yl)acryloyl)piperazin-2-yl)acetonitrile

To a solution of 2-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-1-((E)-3-(azetidin-2-yl)acryloyl)piperazin-2-yl)acetonitrile (40 mg, 48.44 umol, trifluoroacetate salt) in methanol (2 mL) was added formaldehyde (3.93 mg, 48.44 umol, 37% purity), acetic acid (290.87 ug, 4.84 umol) and sodium cyanoborohydride (9.13 mg, 145.31 umol). The mixture was stirred at 20° C. for 1 h. The reaction mixture was concentrated in vacuo. The crude product was purified by reverse phase prep-HPLC (column: Waters Xbridge Prep OBD C18 150*40 mm*10 um; mobile phase: [water (NH₄HCO₃)-ACN]; B %: 40%-70%, 8 min) affording 2-((2S)-4-(7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)-1-((E)-3-(1-methylazetidin-2-yl)acryloyl)piperazin-2-yl)acetonitrile (6.56 mg, 18.66%) as a yellow oil: ¹H NMR (400 MHz, Acetonitrile-d3) δ 6.87-6.71 (m, 1H), 6.68-6.48 (m, 1H), 6.28 (s, 1H), 5.38-5.15 (m, 3H), 5.08 (br d, J=5.4 Hz, 0.5H), 4.67-4.48 (m, 0.5H), 4.11-3.78 (m, 5H), 3.62-3.52 (m, 1H), 3.40-3.09 (m, 6H), 3.09-2.93 (m, 4H), 2.92-2.79 (m, 4H), 2.78-2.64 (m, 2H), 2.46-2.36 (m, 4H), 2.27 (s, 3H), 2.17-2.12 (m, 3H), 2.08 (br s, 1H), 2.04-1.99 (m, 1H), 1.92-1.77 (m, 3H), 0.80 (dd, J=2.3, 6.4 Hz, 3H).

LCMS (5% to 95% acetonitrile in water+0.03% ammonium bicarbonate over 6 min); retention time 2.790 min, ESI+found [M+H]=726.4.

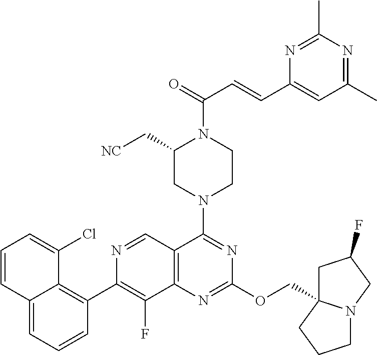 [SMILES: Cc1cc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)nc(C)n1]

### Example 69 (Method 1): 2-((S)-4-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2,6-dimethylpyrimidin-4-yl)acryloyl)piperazin-2-yl)acetonitrile

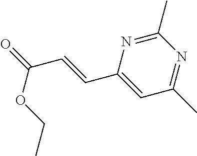

### Step 1: ethyl (E)-3-(2,6-dimethylpyrimidin-4-yl)acrylate

A mixture of 4-chloro-2,6-dimethyl-pyrimidine (4 g, 28.05 mmol), ethyl (E)-3-(4,4,5,5-tetramethyl-1,3,2-dioxaborolan-2-yl)prop-2-enoate (7.61 g, 33.66 mmol), potassium phosphate (17.86 g, 84.16 mmol) and chloro(2-dicyclohexylphosphino-2′,4′,6′-triisopropyl-1,1′-biphenyl)[2-(2′-amino-1,1′-biphenyl)]palladium(II) (2.21 g, 2.81 mmol) in dioxane (60 mL) and water (20 mL) was degassed and purged with nitrogen 3 times. The mixture was stirred at 100° C. for 2 h under a nitrogen atmosphere. The reaction mixture was diluted with water (30 mL) and extracted with ethyl acetate (3×30 mL). The combined organic layers were dried over sodium sulphate and concentrated in vacuo. The residue was purified by column chromatography (silica gel, 100-200 mesh, 0-100% ethyl acetate in petroleum ether) affording ethyl (E)-3-(2,6-dimethylpyrimidin-4-yl)prop-2-enoate (5 g, 86.42%) as a yellow oil: ¹H NMR (400 MHz, Chloroform-d) δ 7.52 (d, J=15.6 Hz, 1H), 7.12-7.02 (m, 2H), 4.29 (q, J=7.1 Hz, 2H), 2.72 (s, 3H), 2.53 (s, 3H), 1.27 (d, J=1.4 Hz, 3H). LCMS Rt=0.564 min, m/z=207.1 [M+H]⁺.

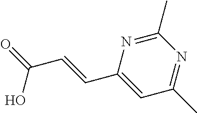

### Step 2: (E)-3-(2,6-dimethylpyrimidin-4-yl)acrylic acid

The hydrolysis reaction was prepared in a similar fashion to Method #1, Step 3. The crude residue was diluted with a (5:1) mixture of petroleum ether and ethyl acetate (20 mL), and the resulting precipitate was filtered affording (E)-3-(2,6-dimethylpyrimidin-4-yl)prop-2-enoic acid (3 g, crude) as a yellow solid, which was used in the next step without further purification. LCMS Rt=0.349 min, m/z=179.1 [M+H]⁺.

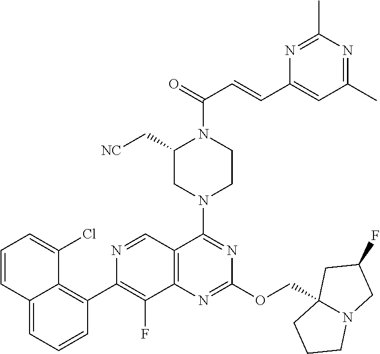 [SMILES: Cc1cc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)nc(C)n1]

### Step 3: 2-((S)-4-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2,6-dimethylpyrimidin-4-yl)acryloyl)piperazin-2-yl)acetonitrile

The amide coupling reaction was prepared in a similar fashion to Method #1, Step 4. The crude product was purified by reverse phase prep-HPLC (column: Waters Xbridge BEH C18 250*50 mm*10 um; mobile phase: [water (NH₄HCO₃)-ACN]; B %: 35%-55%, 10 min) affording 2-((S)-4-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2,6-dimethylpyrimidin-4-yl)acryloyl)piperazin-2-yl)acetonitrile (80.53 mg, 9.92%) as a yellow solid: ¹H NMR (400 MHz, Acetonitrile-d3) δ 9.17-9.11 (m, 1H), 8.18-8.10 (m, 1H), 8.07-7.99 (m, 1H), 7.72-7.59 (m, 4H), 7.56-7.48 (m, 1H), 7.47-7.40 (m, 1H), 7.33-7.19 (m, 1H), 5.41-5.15 (m, 1H), 5.14-4.82 (m, 1H), 4.58-4.44 (m, 2H), 4.28-4.21 (m, 1H), 4.20-4.07 (m, 2H), 3.96-3.70 (m, 2H), 3.22-3.00 (m, 4H), 2.99-2.85 (m, 3H), 2.65-2.56 (m, 3H), 2.49-2.43 (m, 3H), 2.23-2.19 (m, 2H), 1.87-1.73 (m, 4H).

LCMS (5% to 95% acetonitrile in water+0.03% ammonium bicarbonate over 6 min); retention time 2.802 min, ESI+found [M+H]=750.3.

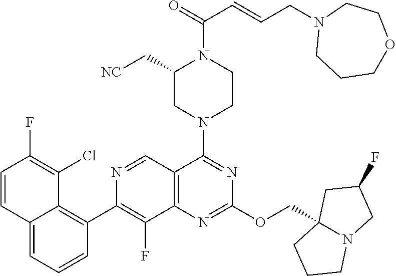 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCCOCC1]

### Example 70 (Method 21): 2-((S)-1-((E)-4-(1,4-oxazepan-4-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

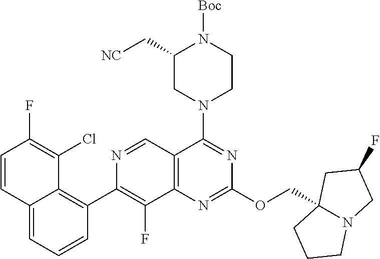 [SMILES: CC(C)(C)OC(=O)N1CCN(c2nc(OC[C@@]34CCCN3C[C@H](F)C4)nc3c(F)c(-c4cccc5ccc(F)c(Cl)c45)ncc23)C[C@@H]1CC#N]

### Step 1: tert-butyl (S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate

The Suzuki reaction was prepared in a similar fashion to Method #16, Step 7. The resulting residue was purified by column chromatography (silica gel, 100-200 mesh, 10% ethyl acetate in petroleum ether) affording tert-butyl (S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (2.6 g, 98.61%) as a yellow oil. LCMS Rt=0.762 min, m/z=708.3 [M+H]⁺.

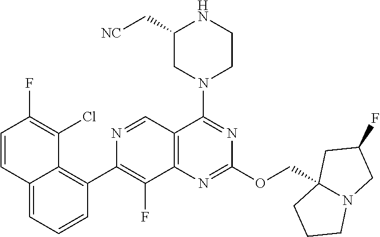 [SMILES: N#CC[C@H]1CN(c2nc(OC[C@@]34CCCN3C[C@H](F)C4)nc3c(F)c(-c4cccc5ccc(F)c(Cl)c45)ncc23)CCN1]

### Step 2: 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

To a solution of tert-butyl (S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (100 mg, 141.21 umol) in dichloromethane (2 mL) was added trifluoroacetic acid (1 mL). The mixture was stirred at 25° C. for 0.5 h. The reaction mixture was concentrated in vacuo affording 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (100 mg, crude, trifluoroacetate salt) as a white solid, which was used in the next step without further purification. LCMS Rt=0.626 min, m/z=608.2 [M+H]⁺.

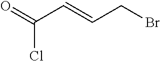

### Step 3: (E)-4-bromobut-2-enoyl chloride

To a solution of (E)-4-bromobut-2-enoic acid (100 mg, 606.12 umol) in dichloromethane (50 mL) was added N,N-dimethylformamide (4.43 mg, 60.61 umol) and oxalyl dichloride (92.32 mg, 727.34 umol) at 0° C. The mixture was stirred at 20° C. for 1 h. The reaction mixture was concentrated in vacuo affording (E)-4-bromobut-2-enoyl chloride (230 mg, crude) as a yellow oil, which was used in the next step without further purification.

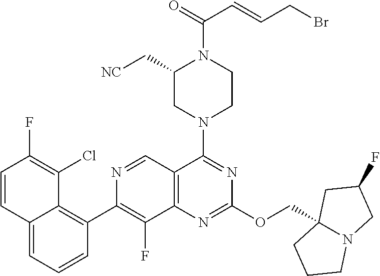 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CBr]

### Step 4: 2-((S)-1-((E)-4-bromobut-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

To a solution of 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (100 mg, 164.46 umol) in tetrahydrofuran (2 mL) and water (0.5 mL) was added sodium hydrogencarbonate (41.45 mg, 493.38 umol) and (E)-4-bromobut-2-enoyl chloride (90.50 mg, 493.38 umol). The mixture was stirred at 0° C. for 1 h affording a solution of 2-((S)-1-((E)-4-bromobut-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl) in acetonitrile as a yellow liquid, which was used in the next step without further purification. LCMS Rt=0.730 min, m/z=754.1 [M+H]⁺.

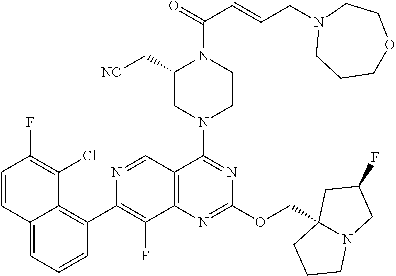 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCCOCC1]

### Step 5: 2-((S)-1-((E)-4-(1,4-oxazepan-4-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

To a solution of 1,4-oxazepane hydrochloride (113.00 mg, 821.16 umol) in tetrahydrofuran (2 mL) was added N,N-diisopropylethylamine (212.25 mg, 1.64 mmol) and 2-((S)-1-((E)-4-bromobut-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (124 mg, 164.23 umol). The mixture was stirred at 0° C. for 1 h. The reaction mixture was concentrated in vacuo and the resulting residue was purified by reverse phase HPLC (column: Waters Xbridge Prep OBD C18 150*40 mm*10 um; mobile phase: [water (NH₄HCO₃)-ACN]; gradient: 30%-60% B over 8 min) affording 2-((S)-1-((E)-4-(1,4-oxazepan-4-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (83.9 mg, 59.08%) as a yellow amorphous solid: ¹H NMR (400 MHz, Acetonitrile-d3) δ 9.17-9.08 (m, 1H), 8.18-8.11 (m, 1H), 8.07 (dd, J=5.7, 8.9 Hz, 1H), 7.71-7.62 (m, 2H), 7.52 (dt, J=1.8, 8.9 Hz, 1H), 6.79 (td, J=5.8, 15.2 Hz, 1H), 6.68-6.45 (m, 1H), 5.36-5.11 (m, 1H), 5.08-4.88 (m, 1H), 4.62-4.30 (m, 3H), 4.27-4.19 (m, 1H), 4.18-4.10 (m, 1H), 4.05-3.87 (m, 1H), 3.86-3.69 (m, 4H), 3.68-3.61 (m, 2H), 3.30 (br d, J=5.1 Hz, 2H), 3.20-3.10 (m, 2H), 3.06 (s, 1H), 2.95-2.82 (m, 3H), 2.74-2.61 (m, 4H), 2.21-2.17 (m, 1H), 2.11-2.02 (m, 2H), 1.92-1.80 (m, 5H).

LCMS (5% to 95% acetonitrile in water+0.03% ammonium bicarbonate over 6 min); retention time 2.771 min, ESI+found [M+H]=775.3.

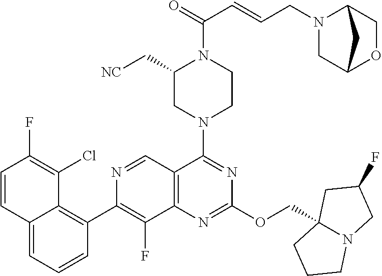 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@@H]2C[C@H]1CO2]

### Example 71 (Method 21): 2-((S)-1-((E)-4-((1S,4S)-2-oxa-5-azabicyclo[2.2.1]heptan-5-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The residue was purified by reverse phase HPLC (column: Waters Xbridge Prep OBD C18 150*40 mm*10 um; mobile phase: [water (NH₄HCO₃)-ACN]; B %: 25%-65%, 8 min) affording 2-((S)-1-((E)-4-((1S,4S)-2-oxa-5-azabicyclo[2.2.1]heptan-5-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (189.92 mg, 13.34%) as a yellow solid: ¹H NMR (400 MHz, Acetonitrile-d3) δ 9.14-9.06 (m, 1H), 8.17-8.12 (m, 1H), 8.07 (dd, J=5.8, 8.6 Hz, 1H), 7.71-7.62 (m, 2H), 7.52 (dt, J=1.7, 8.9 Hz, 1H), 6.78 (td, J=5.2, 14.9 Hz, 1H), 6.56 (br s, 1H), 5.38-5.15 (m, 1H), 5.09-4.68 (m, 1H), 4.60-4.40 (m, 2H), 4.32 (br s, 1H), 4.27-4.18 (m, 1H), 4.15-4.08 (m, 1H), 4.07-3.97 (m, 1H), 3.89 (br d, J=7.5 Hz, 1H), 3.83-3.75 (m, 1H), 3.56 (br d, J=7.3 Hz, 1H), 3.49-3.41 (m, 1H), 3.40-3.27 (m, 2H), 3.18-3.10 (m, 2H), 3.06 (s, 1H), 2.92-2.86 (m, 2H), 2.84-2.75 (m, 1H), 2.59-2.43 (m, 1H), 2.22 (br s, 1H), 2.17-2.10 (m, 2H), 2.09-1.96 (m, 2H), 1.91 (br s, 1H), 1.87 (br dd, J=6.6, 10.8 Hz, 3H), 1.80 (br d, J=9.7 Hz, 1H), 1.64 (br d, J=9.0 Hz, 1H).

LCMS (5% to 95% acetonitrile in water+0.03% ammonium bicarbonate over 6 min); retention time 2.868 min, ESI+found [M+H]=773.3.

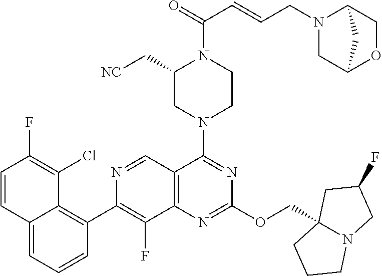 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@H]2C[C@@H]1CO2]

### Example 72 (Method 21): 2-((S)-1-((E)-4-((1R,4R)-2-oxa-5-azabicyclo[2.2.1]heptan-5-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The residue was purified by reverse phase HPLC (column: Waters Xbridge BEH C18 250*50 mm*10 um; mobile phase: [water (NH₄HCO₃)-ACN]; B %: 30%-60%, 10 min) affording 2-((S)-1-((E)-4-((1R,4R)-2-oxa-5-azabicyclo[2.2.1]heptan-5-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (263.75 mg, 24.85%) as a white solid: ¹H NMR (400 MHz, Acetonitrile-d3) δ 9.15-9.08 (m, 1H), 8.16-8.05 (m, 2H), 7.70-7.59 (m, 2H), 7.55-7.40 (m, 1H), 6.85-6.73 (m, 1H), 6.68-6.48 (m, 1H), 5.37-5.16 (m, 1H), 5.07-4.70 (m, 1H), 4.55-4.43 (m, 2H), 4.32 (s, 1H), 4.26-4.20 (m, 1H), 4.16-4.09 (m, 1H), 4.07-3.97 (m, 1H), 3.90 (br d, J=7.4 Hz, 1H), 3.81-3.74 (m, 1H), 3.59-3.53 (m, 1H), 3.47-3.41 (m, 1H), 3.39-3.35 (m, 1H), 3.20-3.12 (m, 2H), 3.09-3.05 (m, 1H), 2.91-2.79 (m, 3H), 2.82 (br d, J=10.4 Hz, 1H), 2.51 (br d, J=10.0 Hz, 1H), 2.24-2.15 (m, 3H), 2.12-2.08 (m, 2H), 2.08-2.00 (m, 1H), 1.92-1.83 (m, 3H), 1.80 (br d, J=10.0 Hz, 1H), 1.64 (br d, J=9.6 Hz, 1H).

LCMS (5% to 95% acetonitrile in water+0.03% ammonium bicarbonate over 6 min); retention time 2.720 min, ESI+found [M+H]=773.3.

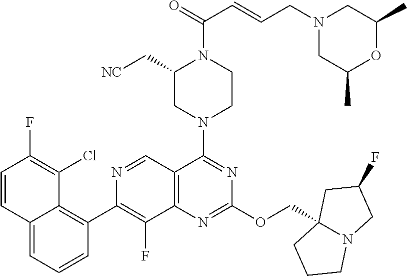 [SMILES: C[C@@H]1CN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C[C@H](C)O1]

### Example 73 (Method 21): 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-((2S,6R)-2,6-dimethylmorpholino)but-2-enoyl)piperazin-2-yl)acetonitrile

The substitution reaction was prepared in a similar fashion to Method #21, Step 5. The residue was purified by reverse phase HPLC (neutral conditions: Waters Xbridge BEH C18 250*50 mm*10 um; mobile phase: [water (NH₄HCO₃)-ACN]; B %: 40%-60%, 10 min) affording 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-((2S,6R)-2,6-dimethylmorpholino)but-2-enoyl)piperazin-2-yl)acetonitrile (356.88 mg, 32.38%) as a yellow amorphous solid: ¹H NMR (400 MHz, Acetonitrile-d3) δ 9.24-9.11 (m, 1H), 8.22-8.15 (m, 1H), 8.11 (dd, J=5.8, 9.1 Hz, 1H), 7.80-7.64 (m, 2H), 7.60-7.40 (m, 1H), 6.87-6.75 (m, 1H), 6.72-6.52 (m, 1H), 5.42-5.21 (m, 1H), 5.12-4.70 (m, 1H), 4.64-4.36 (m, 2H), 4.32-4.23 (m, 1H), 4.21-4.14 (m, 1H), 4.13-3.79 (m, 2H), 3.70-3.48 (m, 3H), 3.24-3.08 (m, 5H), 2.96-2.89 (m, 2H), 2.77 (br d, J=10.4 Hz, 2H), 2.28-2.23 (m, 1H), 2.18-2.02 (m, 3H), 1.91 (br dd, J=6.9, 10.9 Hz, 4H), 1.72 (t, J=10.6 Hz, 2H), 1.11 (d, J=6.4 Hz, 6H).

LCMS (5% to 95% acetonitrile in water+0.03% ammonium bicarbonate over 6 min); retention time 3.037 min, ESI+found [M+H]=789.3.

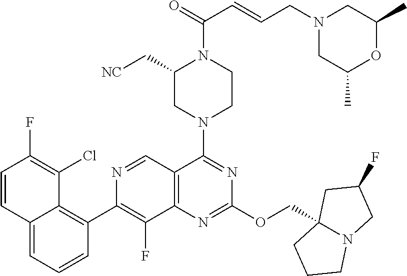 [SMILES: C[C@@H]1CN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C[C@@H](C)O1]

### Example 74 (Method 21): 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-((2R,6R)-2,6-dimethylmorpholino)but-2-enoyl)piperazin-2-yl)acetonitrile

The substitution reaction was prepared in a similar fashion to Method #21, Step 5. The residue was purified by reverse phase HPLC (column: Waters Xbridge BEH C18 250*50 mm*10 um; mobile phase: [water (NH₄HCO₃)-ACN]; B %: 40%-60%, 10 min) affording 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-((2R,6R)-2,6-dimethylmorpholino)but-2-enoyl)piperazin-2-yl)acetonitrile (257.09 mg, 30.67%) as a yellow solid: ¹H NMR (400 MHz, Acetonitrile-d3) δ 9.18-9.09 (m, 1H), 8.19-8.15 (m, 1H), 8.10 (ddd, J=1.1, 5.7, 9.1 Hz, 1H), 7.74-7.66 (m, 2H), 7.55 (dt, J=1.9, 8.9 Hz, 1H), 6.86-6.51 (m, 2H), 5.40-5.21 (m, 1H), 5.09-4.69 (m, 1H), 4.55-4.39 (m, 2H), 4.28-4.23 (m, 1H), 4.18-4.13 (m, 1H), 3.99 (dt, J=3.1, 6.1 Hz, 3H), 3.73 (br s, 3H), 3.23-3.14 (m, 2H), 3.12-3.04 (m, 3H), 2.99-2.85 (m, 3H), 2.48 (br dd, J=2.1, 10.9 Hz, 2H), 2.23-2.19 (m, 2H), 2.14 (br s, 1H), 2.13 (br s, 1H), 2.10-2.06 (m, 1H), 1.93-1.84 (m, 3H), 1.21 (d, J=6.5 Hz, 6H).

LCMS (5% to 95% acetonitrile in water+0.03% ammonium bicarbonate over 6 min); retention time 3.070 min, ESI+found [M+H]=789.3.

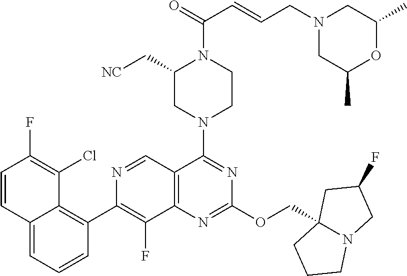 [SMILES: C[C@H]1CN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C[C@H](C)O1]

### Example 75 (Method 21): 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-((2S,6S)-2,6-dimethylmorpholino)but-2-enoyl)piperazin-2-yl)acetonitrile

The substitution reaction was prepared in a similar fashion to Method #21, Step 5. The residue was purified by reverse phase HPLC (neutral conditions, column: Waters Xbridge Prep OBD C18 150*40 mm*10 um; mobile phase: [water (NH₄HCO₃)-ACN]; B %: 35%-65%, 8 min) affording 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-((2S,6S)-2,6-dimethylmorpholino)but-2-enoyl)piperazin-2-yl)acetonitrile (8.4 mg, 15.64%) as a pale yellow amorphous solid: ¹H NMR (400 MHz, Acetonitrile-d3) δ 9.17-9.13 (m, 1H), 8.19-8.14 (m, 1H), 8.09 (dd, J=5.7, 9.1 Hz, 1H), 7.73-7.67 (m, 2H), 7.55 (dt, J=1.8, 8.9 Hz, 1H), 6.84-6.75 (m, 1H), 6.60 (br s, 1H), 5.41-5.22 (m, 1H), 5.08-4.69 (m, 1H), 4.60-4.43 (m, 2H), 4.32-4.25 (m, 1H), 4.23-4.16 (m, 1H), 4.06-3.92 (m, 3H), 3.87-3.46 (m, 3H), 3.28-3.18 (m, 2H), 3.15-3.05 (m, 3H), 2.99-2.87 (m, 3H), 2.47 (br d, J=10.8 Hz, 2H), 2.32-2.21 (m, 4H), 2.13-2.09 (m, 1H), 1.96-1.83 (m, 3H), 1.21 (d, J=6.4 Hz, 6H).

LCMS (5% to 95% acetonitrile in water+0.03% ammonium bicarbonate over 6 min); retention time 3.063 min, ESI+found [M+H]=789.3.

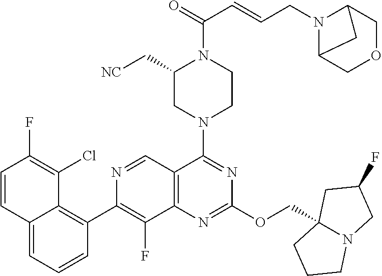 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C2COCC1C2]

### Example 76 (Method 21): 2-((2S)-1-((E)-4-(3-oxa-6-azabicyclo[3.1.1]heptan-6-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The residue was purified by reverse phase HPLC (neutral condition, column: Waters Xbridge Prep OBD C18 150*40 mm*10 um; mobile phase: [water (NH₄HCO₃)-ACN]; B %: 30%-60%, 8 min) affording 2-((2S)-1-((E)-4-(3-oxa-6-azabicyclo[3.1.1]heptan-6-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (8.00 mg, 14.20%) as a yellow amorphous solid: ¹H NMR (400 MHz, Acetonitrile-d3) δ 9.08-9.01 (m, 1H), 8.11-8.04 (m, 1H), 8.03-7.98 (m, 1H), 7.64-7.58 (m, 2H), 7.46 (dt, J=1.8, 8.9 Hz, 1H), 6.82-6.68 (m, 1H), 6.65-6.41 (m, 1H), 5.31-5.10 (m, 1H), 4.99-4.61 (m, 1H), 4.49-4.34 (m, 2H), 4.20-4.13 (m, 1H), 4.11-4.02 (m, 3H), 3.81-3.66 (m, 2H), 3.59 (br d, J=10.8 Hz, 2H), 3.40 (br s, 3H), 3.11-3.05 (m, 2H), 3.00 (s, 1H), 2.88-2.74 (m, 3H), 2.54 (br d, J=6.1 Hz, 1H), 2.14-2.09 (m, 4H), 2.04 (d, J=2.9 Hz, 1H), 2.02-1.97 (m, 1H), 1.86-1.76 (m, 3H), 1.73 (br d, J=8.3 Hz, 1H)).

LCMS (5% to 95% acetonitrile in water+0.03% ammonium bicarbonate over 6 min); retention time 2.874 min, ESI+found [M+H]=773.3

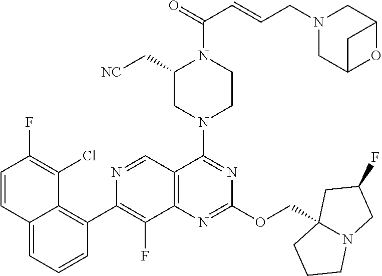 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2CC(C1)O2]

### Example 77 (Method 21): 2-((2S)-1-((E)-4-(6-oxa-3-azabicyclo[3.1.1]heptan-3-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The residue was purified by reverse phase HPLC (column: Waters Xbridge BEH C18 100*30 mm*10 um; mobile phase: [water (NH₄HCO₃)-ACN]; B %: 40%-70%, 8 min) affording 2-((2S)-1-((E)-4-(6-oxa-3-azabicyclo[3.1.1]heptan-3-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (9.2 mg, 17.84%) as a white solid: ¹H NMR (Acetonitrile-d3) δ 9.17-9.12 (m, 1H), 8.20-8.13 (m, 1H), 8.11-8.04 (m, 1H), 7.72-7.66 (m, 2H), 7.57-7.51 (m, 1H), 6.86 (dt, J=14.9, 6.1 Hz, 1H), 6.64 (br s, 1H), 5.39-5.20 (m, 1H), 4.47-4.40 (m, 3H), 4.27-4.22 (m, 1H), 4.18-4.13 (m, 1H), 3.90-3.69 (m, 2H), 3.46-3.40 (m, 2H), 3.19-3.13 (m, 2H), 3.12-3.06 (m, 3H), 2.97-2.90 (m, 3H), 2.75 (br d, J=11.0 Hz, 2H), 2.39-2.19 (m, 4H), 2.17-2.03 (m, 4H), 1.95-1.76 (m, 4H).

LCMS (5% to 95% acetonitrile in water+0.03% ammonium bicarbonate over 6 min); retention time 1.954 min, ESI+found [M+H]=773.3.

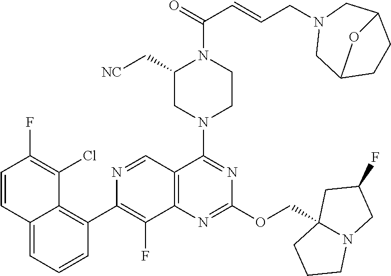 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2CCC(C1)O2]

### Example 78 (Method 21): 2-((2S)-1-((E)-4-(8-oxa-3-azabicyclo[3.2.1]octan-3-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The residue was purified by reverse phase HPLC (column: Waters Xbridge Prep OBD C18 150*40 mm*10 um; mobile phase: [water (NH₄HCO₃)-ACN]; B %: 35%-65%, 8 min) affording 2-((2S)-1-((E)-4-(8-oxa-3-azabicyclo[3.2.1]octan-3-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (173.35 mg, 33.25%) as a white solid: ¹H NMR (400 MHz, Acetonitrile-d3) δ 9.15 (s, 1H), 8.20-8.14 (m, 1H), 8.13-8.07 (m, 1H), 7.75-7.66 (m, 2H), 7.58-7.52 (m, 1H), 6.84-6.50 (m, 2H), 5.40-5.16 (m, 1H), 4.58-4.39 (m, 2H), 4.32-4.06 (m, 5H), 4.00-3.75 (m, 2H), 3.27-3.16 (m, 2H), 3.15-3.09 (m, 3H), 3.01-2.84 (m, 3H), 2.69-2.54 (m, 2H), 2.28 (br d, J=10.8 Hz, 2H), 2.24-2.19 (m, 2H), 2.14 (br s, 2H), 1.93-1.88 (m, 4H), 1.85-1.78 (m, 4H).

LCMS (5% to 95% acetonitrile in water+0.03% ammonium bicarbonate over 6 min); retention time 3.018 min, ESI+found [M+H]=787.3.

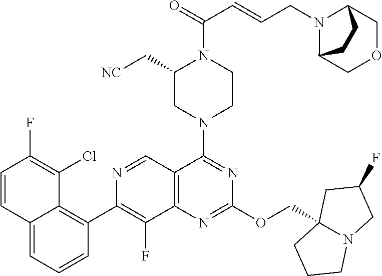

### Example 79 (Method 21): 2-((S)-1-((E)-4-((1R,5S)-3-oxa-8-azabicyclo[3.2.1]octan-8-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The residue was purified by reverse phase HPLC (column: Waters Xbridge Prep OBD C18 150*40 mm*10 um; mobile phase: [water (NH₄HCO₃)-ACN]; B %: 35%-65%, 8 min) affording 2-((S)-1-((E)-4-((1R,5S)-3-oxa-8-azabicyclo[3.2.1]octan-8-yl)but-2-enoyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (308.14 mg, 26.80%) as a pale yellow solid: ¹H NMR (400 MHz, Acetonitrile-d3) δ 9.13-9.09 (m, 1H), 8.16-8.11 (m, 1H), 8.06 (dd, J=5.9, 8.8 Hz, 1H), 7.70-7.63 (m, 2H), 7.51 (dt, J=1.6, 8.9 Hz, 1H), 6.85-6.72 (m, 1H), 6.66-6.52 (m, 1H), 5.36-5.16 (m, 1H), 4.58-4.40 (m, 2H), 4.25-4.19 (m, 1H), 4.16-4.08 (m, 1H), 4.05-3.70 (m, 3H), 3.62 (br d, J=10.0 Hz, 3H), 3.45 (br d, J=9.6 Hz, 2H), 3.18-3.11 (m, 2H), 3.10-3.05 (m, 3H), 3.02 (br s, 2H), 2.91-2.85 (m, 2H), 2.20-2.15 (m, 3H), 2.13-2.08 (m, 1H), 2.08-2.02 (m, 1H), 1.92-1.85 (m, 4H), 1.84-1.76 (m, 3H).

LCMS (5% to 95% acetonitrile in water+0.03% ammonium bicarbonate over 6 min); retention time 2.964 min, ESI+found [M+H]=787.3.

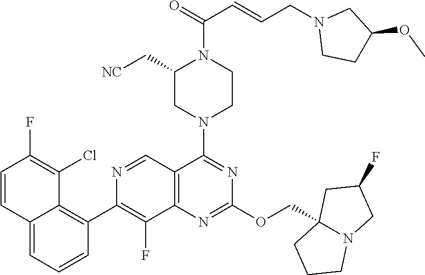 [SMILES: CO[C@H]1CCN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C1]

### Example 80 (Method 21): 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-((S)-3-methoxypyrrolidin-1-yl)but-2-enoyl)piperazin-2-yl)acetonitrile

The substitution reaction was prepared in a similar fashion to Method #21, Step 5. The residue was purified by reverse phase HPLC (column: Waters Xbridge BEH C18 250*50 mm*10 um; mobile phase: [water (NH₄HCO₃)-ACN]; B %: 45%-75%, 10 min) affording 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-((S)-3-methoxypyrrolidin-1-yl)but-2-enoyl)piperazin-2-yl)acetonitrile (97.08 mg, 16.65%) as a yellow solid: ¹H NMR (400 MHz, Acetonitrile-d3) δ 9.33-9.06 (m, 1H), 8.19-8.13 (m, 1H), 8.10 (dd, J=6.3, 8.9 Hz, 1H), 7.81-7.62 (m, 2H), 7.60-7.38 (m, 1H), 6.94-6.76 (m, 1H), 6.68-6.48 (m, 1H), 5.43-5.16 (m, 1H), 5.11-4.70 (m, 1H), 4.65-4.34 (m, 3H), 4.30-4.21 (m, 1H), 4.20-4.13 (m, 1H), 4.09-3.97 (m, 1H), 3.97-3.89 (m, 1H), 3.86-3.53 (m, 3H), 3.37-3.27 (m, 1H), 3.24 (s, 3H), 3.20-3.12 (m, 2H), 3.09 (s, 1H), 2.99-2.82 (m, 3H), 2.77-2.62 (m, 2H), 2.57 (dd, J=3.1, 10.1 Hz, 1H), 2.52-2.42 (m, 1H), 2.14-2.01 (m, 4H), 1.94-1.88 (m, 2H), 1.88-1.70 (m, 2H).

LCMS (5% to 95% acetonitrile in water+0.03% ammonium bicarbonate over 6 min); retention time 2.808 min, ESI+found [M+H]=775.3.

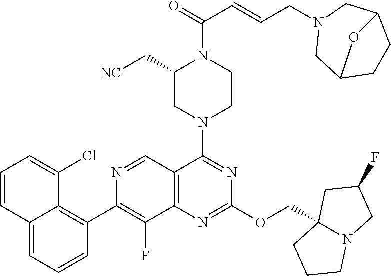 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CCC(C1)O2]

### Example 81 (Method 22): 2-((2S)-1-((E)-4-(8-oxa-3-azabicyclo[3.2.1]octan-3-yl)but-2-enoyl)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile

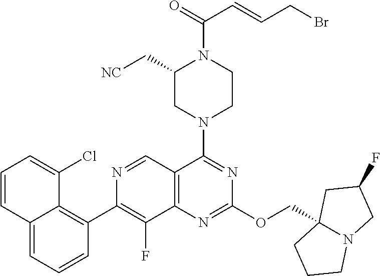 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CBr]

### Step 1: 2-((S)-1-((E)-4-bromobut-2-enoyl)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile

The amide coupling reaction was prepared in a similar fashion to Method #21, Step 4. The reaction was concentrated in vacuo affording 2-((S)-1-((E)-4-bromobut-2-enoyl)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile (619 mg, crude) as a yellow oil, which was used in the next step without further purification. LCMS Rt=0.640 min, m/z=738.2 [M+H]⁺.

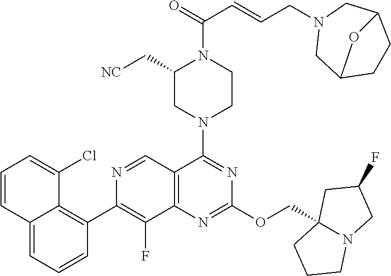 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CCC(C1)O2]

### Step 2: 2-((2S)-1-((E)-4-(8-oxa-3-azabicyclo[3.2.1]octan-3-yl)but-2-enoyl)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile

The substitution reaction was prepared in a similar fashion to Method #21, Step 5. The reaction mixture was purified by reverse phase HPLC (column: Waters Xbridge BEH C18 250*50 mm*10 um; mobile phase: [water (NH₄HCO₃)-ACN]; B %: 40%-60%, 10 min) affording 2-((2S)-1-((E)-4-(8-oxa-3-azabicyclo[3.2.1]octan-3-yl)but-2-enoyl)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile (161 mg, 24.82%) as a yellow amorphous solid: ¹H NMR (400 MHz, Acetonitrile-d3) δ 9.19-9.13 (m, 1H), 8.17 (d, J=7.70 Hz, 1H), 8.06 (d, J=8.07 Hz, 1H), 7.74-7.63 (m, 3H), 7.56 (td, J=7.82, 1.71 Hz, 1H), 6.79 (dt, J=14.52, 5.76 Hz, 1H), 6.66-6.52 (m, 1H), 5.40-5.21 (m, 1H), 5.12-4.96 (m, 1H), 4.62-4.40 (m, 3H), 4.28-4.24 (m, 3H), 4.20-4.14 (m, 1H), 4.08-3.60 (m, 4H), 3.21-3.09 (m, 6H), 2.98-2.89 (m, 3H), 2.64 (br d, J=10.88 Hz, 2H), 2.29 (br d, J=11.00 Hz, 2H), 2.16-2.07 (m, 3H), 1.94-1.87 (m, 3H), 1.86-1.80 (m, 2H).

LCMS (5% to 95% acetonitrile in water+0.03% ammonium bicarbonate over 6 min); retention time 3.014 min, ESI+found [M+H]=769.3.

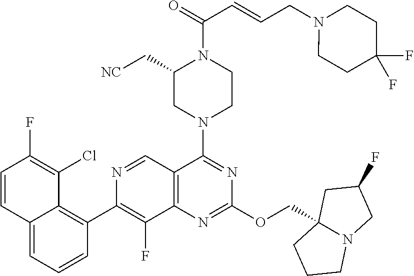 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCC(F)(F)CC1]

### Example 82 (Method 21): 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-(4,4-difluoropiperidin-1-yl)but-2-enoyl)piperazin-2-yl)acetonitrile

The substitution reaction was prepared in a similar fashion to Method #21, Step 5. The residue was purified by reverse phase HPLC (column: Waters Xbridge BEH C18 100*30 mm*10 um; mobile phase: [water (NH₄HCO₃)-ACN]; B %: 40%-70%, 8 min) affording 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-(4,4-difluoropiperidin-1-yl)but-2-enoyl)piperazin-2-yl)acetonitrile (7.93 mg, 37.00%) as a yellow amorphous solid: ¹H NMR (400 MHz, Acetonitrile-d3) δ 9.19-9.08 (m, 1H), 8.19-8.13 (m, 1H), 8.09 (dd, J=5.6, 9.0 Hz, 1H), 7.73-7.66 (m, 2H), 7.55 (dt, J=1.7, 9.0 Hz, 1H), 6.85-6.75 (m, 1H), 6.58 (br d, J=12.9 Hz, 1H), 5.42-5.16 (m, 1H), 5.09-4.71 (m, 1H), 4.61-4.40 (m, 2H), 4.29-4.20 (m, 1H), 4.18-4.10 (m, 1H), 4.09-3.90 (m, 1H), 3.88-3.39 (m, 3H), 3.23 (br d, J=5.4 Hz, 2H), 3.19-3.12 (m, 2H), 3.09 (s, 1H), 2.98-2.84 (m, 3H), 2.58 (br s, 4H), 2.23-2.16 (m, 3H), 2.13 (d, J=2.9 Hz, 1H), 2.10-2.00 (m, 4H), 1.93-1.86 (m, 2H).

LCMS (5% to 95% acetonitrile in water+0.03% ammonium bicarbonate over 6 min); retention time 3.147 min, ESI+found [M+H]=795.3.

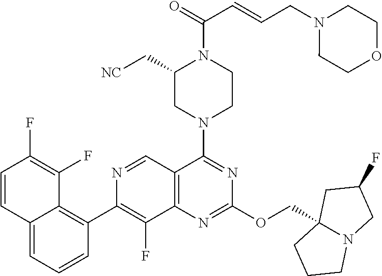 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCOCC1]

### Example 83 (Method 23): 2-((S)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-morpholinobut-2-enoyl)piperazin-2-yl)acetonitrile

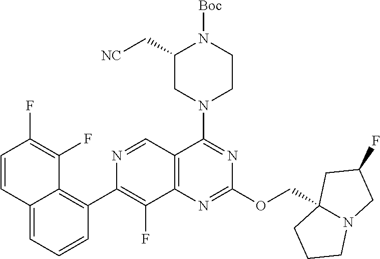 [SMILES: CC(C)(C)OC(=O)N1CCN(c2nc(OC[C@@]34CCCN3C[C@H](F)C4)nc3c(F)c(-c4cccc5ccc(F)c(F)c45)ncc23)C[C@@H]1CC#N]

### Step 1: tert-butyl (S)-2-(cyanomethyl)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazine-1-carboxylate

The Suzuki coupling reaction was prepared in a similar fashion to Method #16, Step 7. The crude product was purified by column chromatography (silica gel, 100-200 mesh, 0-30% ethyl acetate in petroleum ether) affording tert-butyl (S)-2-(cyanomethyl)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazine-1-carboxylate (3 g, 63.02%) as a brown gum. LCMS Rt=0.636 min, m/z=692.3 [M+H]⁺.

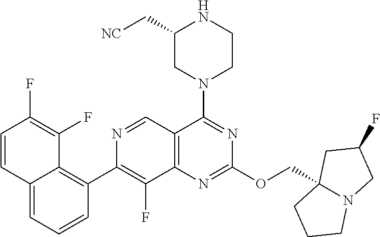 [SMILES: N#CC[C@H]1CN(c2nc(OC[C@@]34CCCN3C[C@H](F)C4)nc3c(F)c(-c4cccc5ccc(F)c(F)c45)ncc23)CCN1]

### Step 2: 2-((S)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The Boc deprotection reaction was prepared in a similar fashion to Method #21, Step 2. The reaction was concentrated in vacuo affording 2-((S)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (2 g, crude, trifluoroacetate salt) as a brown gum, which was used in the next step without further purification. LCMS Rt=0.520 min, m/z=592.2 [M+H]⁺.

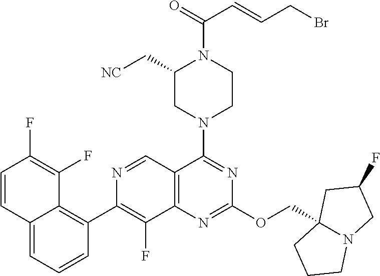 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CBr]

### Step 3: 2-((S)-1-((E)-4-bromobut-2-enoyl)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The acylation reaction was prepared in a similar fashion to Method #21, Step 4. The resulting solution of 2-((S)-1-((E)-4-bromobut-2-enoyl)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl) in acetonitrile (yellow liquid) was used in the next step without further purification. LCMS Rt=0.669 min, m/z=738.2 [M+H]⁺.

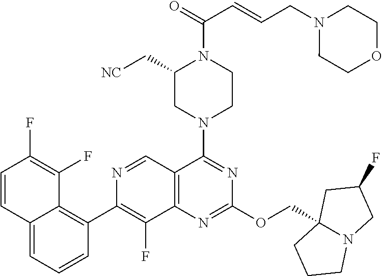 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCOCC1]

### Step 4: 2-((S)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-morpholinobut-2-enoyl)piperazin-2-yl)acetonitrile

The substitution reaction was prepared in a similar fashion to Method #21, Step 5. The residue was purified by reverse phase HPLC (column: Waters Xbridge Prep OBD C18 150*40 mm*10 um; mobile phase: [water (NH₄HCO₃)-ACN]; B %: 20%-50%, 8 min) affording 2-((S)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-morpholinobut-2-enoyl)piperazin-2-yl)acetonitrile (432.7 mg, 21.45%) as a white solid: ¹H NMR (400 MHz, Acetonitrile-d3) δ 8.72 (br s, 1H), 7.69 (br s, 1H), 7.47 (br dd, J=5.1, 7.7 Hz, 1H), 7.27 (br d, J=5.6 Hz, 2H), 7.19-7.05 (m, 1H), 6.46-6.08 (m, 2H), 4.97-4.75 (m, 1H), 4.59 (br s, 1H), 4.13-3.96 (m, 2H), 3.86-3.72 (m, 2H), 3.66-3.30 (m, 3H), 3.22 (br s, 4H), 2.84-2.63 (m, 6H), 2.50 (br s, 3H), 2.01 (br s, 4H), 1.81-1.63 (m, 3H), 1.55-1.44 (m, 3H).

LCMS (5% to 95% acetonitrile in water+0.03% ammonium bicarbonate over 6 min); retention time 2.671 min, ESI+found [M+H]=745.3.

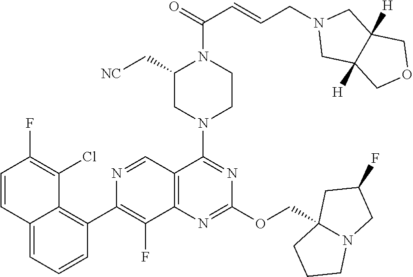 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@H]2COC[C@H]2C1]

### Example 84 (Method 21): 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-((3aR,6aS)-tetrahydro-1H-furo[3,4-c]pyrrol-5(3H)-yl)but-2-enoyl)piperazin-2-yl)acetonitrile

The substitution reaction was prepared in a similar fashion to Method #21, Step 5. The residue was purified by reverse phase HPLC (column: Waters Xbridge BEH C18 100*30 mm*10 um; mobile phase: [water (NH₄HCO₃)-ACN]; B %: 30%-60%, 8 min) affording 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-((3aR,6aS)-tetrahydro-1H-furo[3,4-c]pyrrol-5(3H)-yl)but-2-enoyl)piperazin-2-yl)acetonitrile (21.32 mg, 25.03%) as a yellow oil: ¹H NMR (400 MHz, Acetonitrile-d3) δ 9.16 (br s, 1H), 8.21-8.07 (m, 2H), 7.75-7.67 (m, 2H), 7.56 (dt, J=1.7, 9.0 Hz, 1H), 6.90-6.77 (m, 1H), 6.71-6.50 (m, 1H), 5.41-5.18 (m, 1H), 5.09-4.71 (m, 1H), 4.59-4.43 (m, 2H), 4.29-4.22 (m, 1H), 4.19-4.12 (m, 1H), 4.09-3.92 (m, 1H), 3.88-3.73 (m, 4H), 3.54-3.43 (m, 2H), 3.28-3.15 (m, 4H), 3.10 (s, 1H), 2.96-2.90 (m, 2H), 2.78 (br s, 2H), 2.63-2.55 (m, 2H), 2.45 (br d, J=7.0 Hz, 2H), 2.23-2.17 (m, 3H), 2.14 (br d, J=2.6 Hz, 1H), 2.12-2.05 (m, 1H), 1.96-1.82 (m, 3H).

LCMS (5% to 95% acetonitrile in water+0.03% ammonium bicarbonate over 6 min); retention time 1.009 min, ESI+found [M+H]=787.3.

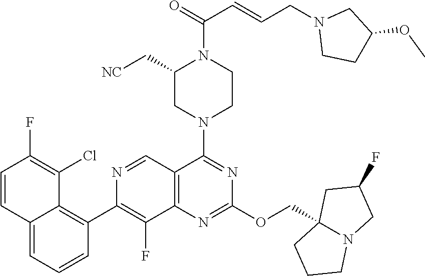 [SMILES: CO[C@@H]1CCN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C1]

### Example 85 (Method 21): 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-((R)-3-methoxypyrrolidin-1-yl)but-2-enoyl)piperazin-2-yl)acetonitrile

The substitution reaction was prepared in a similar fashion to Method #21, Step 5. The residue was purified by reverse phase HPLC (column: Waters Xbridge BEH C18 250*50 mm*10 um; mobile phase: [water (NH₄HCO₃)-ACN]; B %: 40%-60%, 10 min) affording 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-((R)-3-methoxypyrrolidin-1-yl)but-2-enoyl)piperazin-2-yl)acetonitrile (21.32 mg, 25.03%) as a white solid: ¹H NMR (400 MHz, Acetonitrile-d3) δ 9.22-9.07 (m, 1H), 8.18-8.11 (m, 1H), 8.07 (dd, J=6.0, 8.8 Hz, 1H), 7.72-7.63 (m, 2H), 7.52 (dt, J=1.9, 8.9 Hz, 1H), 6.80 (td, J=5.8, 15.3 Hz, 1H), 6.65-6.45 (m, 1H), 5.37-5.16 (m, 1H), 5.09-4.67 (m, 1H), 4.61-4.32 (m, 3H), 4.28-4.19 (m, 1H), 4.17-4.10 (m, 1H), 4.01 (ddd, J=4.7, 7.8, 9.6 Hz, 1H), 3.90 (ddd, J=3.2, 6.7, 10.1 Hz, 2H), 3.83-3.32 (m, 3H), 3.23-3.21 (m, 3H), 3.17-3.12 (m, 2H), 3.07 (s, 1H), 2.98-2.83 (m, 3H), 2.74-2.62 (m, 2H), 2.55 (dd, J=3.2, 10.2 Hz, 1H), 2.49-2.40 (m, 1H), 2.13-2.09 (m, 2H), 2.07-2.00 (m, 2H), 1.92-1.81 (m, 3H), 1.78-1.67 (m, 1H).

LCMS (5% to 95% acetonitrile in water+0.03% ammonium bicarbonate over 6 min); retention time 2.956 min, ESI+found [M+H]=775.3.

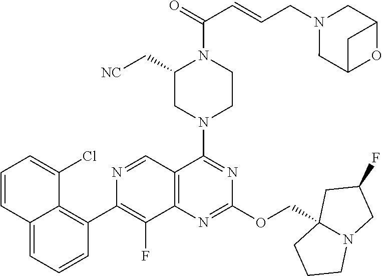 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CC(C1)O2]

### Example 86 (Method 22): 2-((2S)-1-((E)-4-(6-oxa-3-azabicyclo[3.1.1]heptan-3-yl)but-2-enoyl)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile

The substitution reaction was prepared in a similar fashion to Method #21, Step 5. The reaction mixture was purified by reverse phase HPLC (column: Waters Xbridge Prep OBD C18 150*40 mm*10 um; mobile phase: [water (NH₄HCO₃)-ACN]; B %: 30%-60%, 8 min) affording 2-((2S)-1-((E)-4-(6-oxa-3-azabicyclo[3.1.1]heptan-3-yl)but-2-enoyl)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile (426.14 mg, 39.90%) as a yellow oil: ¹H NMR (400 MHz, Chloroform-d) δ 9.07 (s, 1H), 8.02 (d, J=7.5 Hz, 1H), 7.89 (d, J=8.1 Hz, 1H), 7.65-7.52 (m, 3H), 7.47-7.39 (m, 1H), 7.08-6.98 (m, 1H), 6.57-6.44 (m, 1H), 5.38-5.21 (m, 1H), 5.14-4.96 (m, 1H), 4.61-4.42 (m, 4H), 4.38-4.29 (m, 1H), 4.28-4.19 (m, 1H), 4.09-3.76 (m, 3H), 3.45 (br d, J=5.4 Hz, 2H), 3.26 (br d, J=11.8 Hz, 2H), 3.20-3.10 (m, 3H), 3.08-2.95 (m, 3H), 2.90-2.75 (m, 3H), 2.37 (d, J=8.0 Hz, 1H), 2.31-2.25 (m, 1H), 2.23-2.11 (m, 2H), 2.04-1.83 (m, 4H).

LCMS (5% to 95% acetonitrile in water+0.03% ammonium bicarbonate over 6 min); retention time 2.874 min, ESI+found [M+H]=755.3.

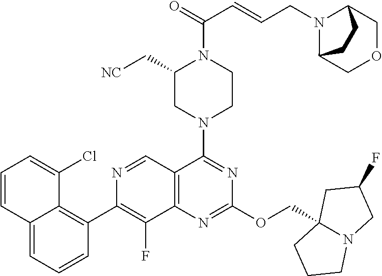

### Example 87 (Method 22): 2-((S)-1-((E)-4-((1R,5S)-3-oxa-8-azabicyclo[3.2.1]octan-8-yl)but-2-enoyl)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile

The substitution reaction was prepared in a similar fashion to Method #21, Step 5. The reaction mixture was purified by reverse phase HPLC (column: Waters Xbridge Prep OBD C18 150*40 mm*10 um; mobile phase: [water (NH₄HCO₃)-ACN]; B %: 25%-65%, 8 min) affording 2-((S)-1-((E)-4-((1R,5S)-3-oxa-8-azabicyclo[3.2.1]octan-8-yl)but-2-enoyl)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile (55.02 mg, 17.53%) as a yellow amorphous solid: ¹H NMR (400 MHz, Chloroform-d) δ 9.14-9.03 (m, 1H), 8.06-7.98 (m, 1H), 7.92-7.85 (m, 1H), 7.66-7.52 (m, 3H), 7.43 (dt, J=2.5, 7.8 Hz, 1H), 7.07-6.94 (m, 1H), 6.65-6.49 (m, 1H), 5.42-5.17 (m, 1H), 5.14-4.68 (m, 1H), 4.63-4.41 (m, 2H), 4.38-4.28 (m, 1H), 4.28-4.18 (m, 1H), 4.16-3.89 (m, 2H), 3.89-3.62 (m, 4H), 3.55 (br d, J=10.1 Hz, 2H), 3.25 (br d, J=9.9 Hz, 2H), 3.22-3.10 (m, 3H), 3.05 (br s, 2H), 2.98 (br dd, J=4.8, 9.2 Hz, 2H), 2.87-2.73 (m, 1H), 2.30-2.24 (m, 1H), 2.23-2.09 (m, 2H), 1.92 (br s, 7H).

LCMS (5% to 95% acetonitrile in water+0.03% ammonium bicarbonate over 6 min); retention time 2.955 min, ESI+found [M+H]=769.3.

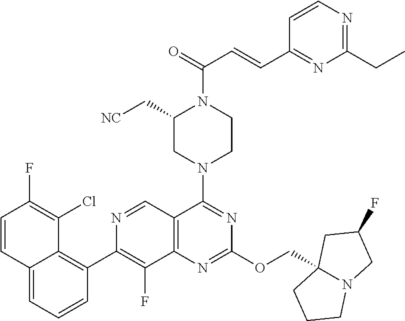 [SMILES: CCc1nccc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1]

### Example 88 (Method 1): 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2-ethylpyrimidin-4-yl)acryloyl)piperazin-2-yl)acetonitrile

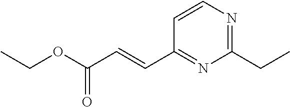

### Step 1: (E)-ethyl 3-(2-ethylpyrimidin-4-yl)acrylate

To a solution of ethyl 2-diethoxyphosphorylacetate (1 g, 4.46 mmol) in acetonitrile (15 mL) was added lithium chloride (945.40 mg, 22.30 mmol) and N,N-diisopropylethylamine (1.73 g, 13.38 mmol), and the mixture was stirred at 20° C. for 10 min. 2-Ethylpyrimidine-4-carboxaldehyde (910.95 mg, 6.69 mmol) was added and stirred at 20° C. for 2 h. Water (20 mL) was added, and the mixture was extracted with ethyl acetate (2×30 mL). The combined organic layers were dried over sodium sulphate and concentrated in vacuo. The residue was purified by column chromatography (silica gel, 100-200 mesh, 0-100% ethyl acetate in petroleum ether) affording (E)-ethyl 3-(2-ethylpyrimidin-4-yl)acrylate (650 mg, 66.84%) as a colorless oil: ¹H NMR (400 MHz, Chloroform-d) δ 8.71 (d, J=5.0 Hz, 1H), 7.56 (d, J=15.8 Hz, 1H), 7.19-7.08 (m, 2H), 4.33-4.25 (m, 2H), 3.01 (q, J=7.5 Hz, 2H), 1.43-1.31 (m, 6H). LCMS Rt=0.665 min, m/z=207.1 [M+H]⁺.

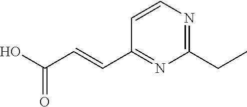

### Step 2: (E)-3-(2-ethylpyrimidin-4-yl)acrylic acid

The hydrolysis reaction was prepared in a similar fashion to Method #1, Step 3. The mixture was filtered and the filter cake was concentrated in vacuo affording (E)-3-(2-ethylpyrimidin-4-yl)prop-2-enoic acid (470 mg, 83.69%) as a white solid: ¹H NMR (400 MHz, Dimethyl sulfoxide-d6) δ 12.94-12.83 (m, 1H), 8.84-8.78 (m, 1H), 7.63-7.59 (m, 1H), 7.56-7.47 (m, 1H), 7.08-6.98 (m, 1H), 2.91 (q, J=7.5 Hz, 2H), 1.28 (t, J=7.6 Hz, 3H). LCMS Rt=0.425 min, m/z=179.1 [M+H]⁺.

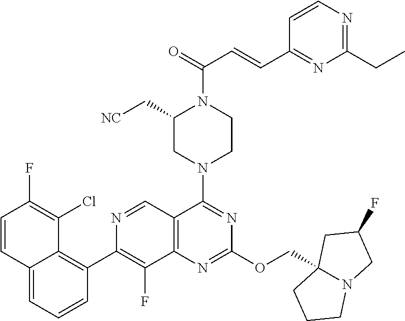 [SMILES: CCc1nccc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1]

### Step 3: 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2-ethylpyrimidin-4-yl)acryloyl)piperazin-2-yl)acetonitrile

The amide coupling reaction was prepared in a similar fashion to Method #1, Step 4. The crude product was purified by reverse phase prep-HPLC (column: Waters Xbridge Prep OBD C18 150*40 mm*10 um; mobile phase: [water (NH₄HCO₃)-ACN]; B %: 35%-65%, 8 min) affording 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2-ethylpyrimidin-4-yl)acryloyl)piperazin-2-yl)acetonitrile (33.75 mg, 13.25%) as a white solid: ¹H NMR (400 MHz, Acetonitrile-d3) δ 9.14 (s, 1H), 8.72 (br d, J=4.3 Hz, 1H), 8.18-8.10 (m, 1H), 8.07 (dd, J=5.7, 8.6 Hz, 1H), 7.86-7.64 (m, 3H), 7.56-7.46 (m, 2H), 7.35 (br d, J=4.3 Hz, 1H), 5.36-5.17 (m, 1H), 5.13-4.81 (m, 1H), 4.62-4.42 (m, 2H), 4.26-4.21 (m, 1H), 4.20-4.05 (m, 2H), 3.99-3.74 (m, 2H), 3.73-3.53 (m, 1H), 3.22-3.11 (m, 2H), 3.11-3.05 (m, 1H), 2.95 (br d, J=7.8 Hz, 3H), 2.93-2.84 (m, 2H), 2.13-2.02 (m, 3H), 1.91-1.76 (m, 3H), 1.41-1.29 (m, 3H).

LCMS (5% to 95% acetonitrile in water+0.03% ammonium bicarbonate over 6 min); retention time 3.071 min, ESI+found [M+H]=768.3.

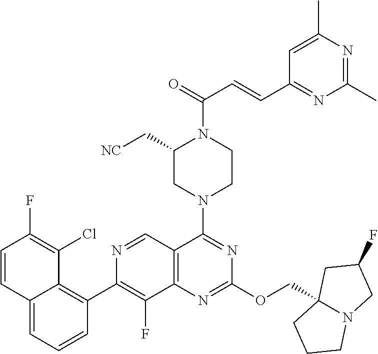 [SMILES: Cc1cc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)nc(C)n1]

### Example 89 (Method 1): 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2,6-dimethylpyrimidin-4-yl)acryloyl)piperazin-2-yl)acetonitrile

The amide coupling reaction was prepared in a similar fashion to Method #1, Step 4. The crude product was purified by reverse phase prep-HPLC (column: Waters Xbridge Prep OBD C18 150*40 mm*10 um; mobile phase: [water (NH₄HCO₃)-ACN]; B %: 35%-65%, 8 min) affording 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2,6-dimethylpyrimidin-4-yl)acryloyl)piperazin-2-yl)acetonitrile (56.29 mg, 28.80%) as a brown solid: ¹H NMR (400 MHz, Acetonitrile-d3) δ 9.16 (s, 1H), 8.26-8.03 (m, 2H), 7.85-7.63 (m, 3H), 7.61-7.37 (m, 2H), 7.26 (br s, 1H), 5.42-5.17 (m, 1H), 5.16-4.80 (m, 1H), 4.66-4.39 (m, 3H), 4.32-4.24 (m, 1H), 4.17 (br d, J=10.3 Hz, 2H), 4.03-3.82 (m, 2H), 3.21-3.14 (m, 2H), 3.10 (s, 1H), 3.04-2.85 (m, 3H), 2.64 (br s, 3H), 2.48 (s, 3H), 2.16-2.03 (m, 3H), 1.90 (br dd, J=6.6, 10.9 Hz, 3H).

LCMS (5% to 95% acetonitrile in water+0.03% ammonium bicarbonate over 6 min); retention time 2.192 min, ESI+found [M+H]=768.3.

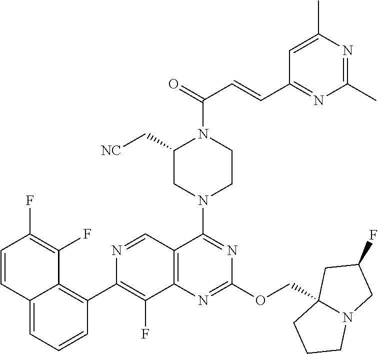 [SMILES: Cc1cc(/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)nc(C)n1]

### Example 90 (Method 1): 2-((S)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2,6-dimethylpyrimidin-4-yl)acryloyl)piperazin-2-yl)acetonitrile

The amide coupling reaction was prepared in a similar fashion to Method #1, Step 4. The crude product was purified by reverse phase prep-HPLC (column: Waters Xbridge Prep OBD C18 150*40 mm*10 um; mobile phase: [water (NH₄HCO₃)-ACN]; B %: 25%-55%, 8 min) affording 2-((S)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2,6-dimethylpyrimidin-4-yl)acryloyl)piperazin-2-yl)acetonitrile (103.12 mg, 26.90%) as a yellow solid: ¹H NMR (400 MHz, Chloroform-d) δ 9.10 (s, 1H), 8.03-7.93 (m, 1H), 7.78-7.68 (m, 2H), 7.68-7.53 (m, 3H), 7.46-7.35 (m, 1H), 7.00 (s, 1H), 5.39-5.20 (m, 1H), 5.19-4.74 (m, 1H), 4.67-4.44 (m, 2H), 4.38-4.31 (m, 1H), 4.24 (br d, J=10.1 Hz, 2H), 4.13-3.96 (m, 1H), 3.94-3.59 (m, 2H), 3.35-3.22 (m, 2H), 3.21-3.15 (m, 1H), 3.11-2.94 (m, 2H), 2.91-2.77 (m, 1H), 2.73 (s, 3H), 2.54 (s, 3H), 2.32-2.23 (m, 1H), 2.22-2.08 (m, 2H), 2.01-1.86 (m, 3H).

LCMS (5% to 95% acetonitrile in water+0.03% ammonium bicarbonate over 6 min); retention time 2.962 min, ESI+found [M+H]=752.3.

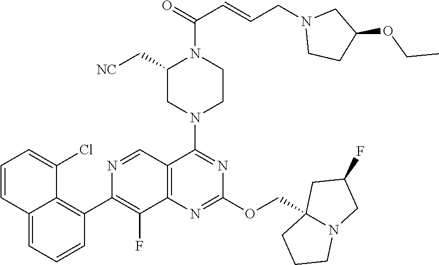 [SMILES: CCO[C@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1]

### Example 91 (Method 22): 2-((S)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)-1-((E)-4-((S)-3-ethoxypyrrolidin-1-yl)but-2-enoyl)piperazin-2-yl)acetonitrile

The substitution reaction was prepared in a similar fashion to Method #21, Step 5. The reaction mixture was purified by reverse phase HPLC (column: Waters Xbridge Prep OBD C18 150*40 mm*10 um; mobile phase: [water (NH₄HCO₃)-ACN]; B %: 30%-60%, 8 min) affording 2-((S)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)-1-((E)-4-((S)-3-ethoxypyrrolidin-1-yl)but-2-enoyl)piperazin-2-yl)acetonitrile (17.55 mg, 16.77%) as a yellow solid: ¹H NMR (400 MHz, Acetonitrile-d3) δ 9.27-9.10 (m, 1H), 8.16 (dd, J=1.1, 8.1 Hz, 1H), 8.05 (d, J=8.1 Hz, 1H), 7.75-7.69 (m, 1H), 7.68-7.61 (m, 2H), 7.55 (dt, J=1.7, 7.8 Hz, 1H), 6.88-6.54 (m, 2H), 5.42-5.21 (m, 1H), 5.10-4.76 (m, 1H), 4.61-4.45 (m, 2H), 4.32-4.26 (m, 1H), 4.24-4.18 (m, 1H), 4.14-3.97 (m, 2H), 3.81 (br s, 3H), 3.48-3.40 (m, 2H), 3.31 (br s, 2H), 3.26-3.19 (m, 2H), 3.15 (br s, 1H), 3.00-2.87 (m, 3H), 2.82-2.71 (m, 2H), 2.55 (br s, 2H), 2.15-2.13 (m, 1H), 2.10 (br s, 1H), 2.08-2.01 (m, 2H), 1.95-1.89 (m, 3H), 1.82-1.75 (m, 1H), 1.15 (t, J=6.9 Hz, 3H).

LCMS (5% to 95% acetonitrile in water+0.03% ammonium bicarbonate over 6 min); retention time 3.039 min, ESI+found [M+H]=771.3.

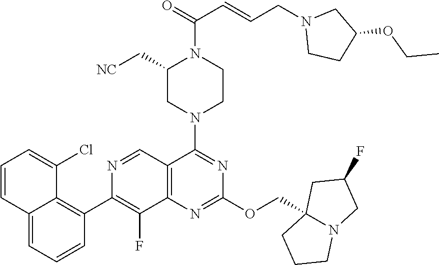 [SMILES: CCO[C@@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1]

### Example 92 (Method 22): 2-((S)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)-1-((E)-4-((R)-3-ethoxypyrrolidin-1-yl)but-2-enoyl)piperazin-2-yl)acetonitrile

The substitution reaction was prepared in a similar fashion to Method #21, Step 5. The reaction mixture was purified by reverse phase HPLC (column: Waters Xbridge Prep OBD C18 150*40 mm*10 um; mobile phase: [water (NH₄HCO₃)-ACN]; B %: 35%-55%, 8 min) affording 2-((S)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)-1-((E)-4-((R)-3-ethoxypyrrolidin-1-yl)but-2-enoyl)piperazin-2-yl)acetonitrile (16.53 mg, 15.80%) as a yellow solid: ¹H NMR (400 MHz, Acetonitrile-d3) δ 9.21-9.10 (m, 1H), 8.17 (d, J=7.6 Hz, 1H), 8.06 (d, J=8.1 Hz, 1H), 7.76-7.70 (m, 1H), 7.69-7.62 (m, 2H), 7.55 (dt, J=1.7, 7.7 Hz, 1H), 6.89-6.76 (m, 1H), 6.48 (br d, J=16.4 Hz, 1H), 5.41-5.20 (m, 1H), 5.13-4.66 (m, 1H), 4.61-4.41 (m, 2H), 4.29-4.22 (m, 1H), 4.21-4.14 (m, 1H), 4.12-3.98 (m, 2H), 3.96-3.50 (m, 3H), 3.48-3.38 (m, 2H), 3.32-3.23 (m, 2H), 3.18 (br d, J=8.4 Hz, 2H), 3.11 (s, 1H), 3.01-2.84 (m, 3H), 2.81-2.63 (m, 2H), 2.61-2.53 (m, 1H), 2.51-2.43 (m, 1H), 2.22 (br s, 1H), 2.15 (br d, J=2.8 Hz, 1H), 2.12-2.03 (m, 2H), 1.95-1.81 (m, 3H), 1.79-1.68 (m, 1H), 1.15 (t, J=7.0 Hz, 3H).

LCMS (5% to 95% acetonitrile in water+0.03% ammonium bicarbonate over 6 min); retention time 3.033 min, ESI+found [M+H]=771.3.

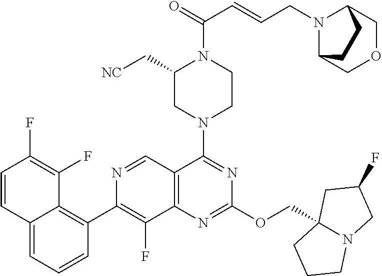

### Example 93 (Method 23): 2-((S)-1-((E)-4-((1R,5S)-3-oxa-8-azabicyclo[3.2.1]octan-8-yl)but-2-enoyl)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The residue was purified by reverse phase HPLC (column: Waters Xbridge Prep OBD C18 150*40 mm*10 um; mobile phase: [water (NH₄HCO₃)-ACN]; B %: 30%-60%, 8 min) affording 2-((S)-1-((E)-4-((1R,5S)-3-oxa-8-azabicyclo[3.2.1]octan-8-yl)but-2-enoyl)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (108.87 mg, 26.08%) as a pale yellow solid: ¹H NMR (400 MHz, Chloroform-d) δ 9.11 (s, 1H), 8.00 (br d, J=7.8 Hz, 1H), 7.78-7.72 (m, 1H), 7.69-7.59 (m, 2H), 7.47-7.38 (m, 1H), 7.07-6.98 (m, 1H), 6.63-6.53 (m, 1H), 5.46-5.19 (m, 1H), 5.07 (br s, 1H), 4.65-4.45 (m, 2H), 4.41-4.22 (m, 2H), 4.15-3.82 (m, 3H), 3.77 (br d, J=10.3 Hz, 2H), 3.60-3.54 (m, 2H), 3.46-3.20 (m, 3H), 3.15 (br d, J=3.9 Hz, 2H), 3.11-2.97 (m, 4H), 2.89-2.75 (m, 1H), 2.36-2.13 (m, 3H), 2.07-1.86 (m, 8H).

LCMS (5% to 95% acetonitrile in water+0.03% ammonium bicarbonate over 6 min); retention time 2.773 min, ESI+found [M+H]=771.3.

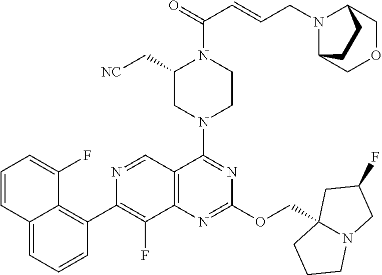

### Example 94 (Method 24): 2-((S)-1-((E)-4-((1R,5S)-3-oxa-8-azabicyclo[3.2.1]octan-8-yl)but-2-enoyl)-4-(8-fluoro-7-(8-fluoronaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

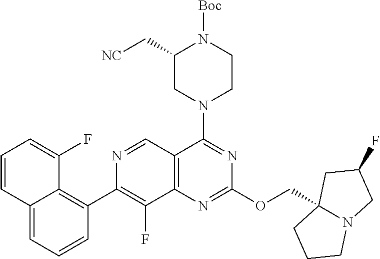 [SMILES: CC(C)(C)OC(=O)N1CCN(c2nc(OC[C@@]34CCCN3C[C@H](F)C4)nc3c(F)c(-c4cccc5cccc(F)c45)ncc23)C[C@@H]1CC#N]

### Step 1: tert-butyl (S)-2-(cyanomethyl)-4-(8-fluoro-7-(8-fluoronaphthalen-1-yl)-2-(((2R,7aS)-2-fluorotetrahydro-1H-pyrrolizin-7a(5H)-yl)methoxy)pyrido[4,3-d]pyrimidin-4-yl)piperazine-1-carboxylate

The Suzuki reaction was prepared in a similar fashion to Method #16, Step 7. The residue was purified by column chromatography (silica gel, 100-200 mesh, 0-100% methanol in dichloromethane) affording tert-butyl (S)-2-(cyanomethyl)-4-(8-fluoro-7-(8-fluoronaphthalen-1-yl)-2-(((2R,7aS)-2-fluorotetrahydro-1H-pyrrolizin-7a(5H)-yl)methoxy)pyrido[4,3-d]pyrimidin-4-yl)piperazine-1-carboxylate (2.2 g, 92.09%) as a yellow oil. LCMS Rt=1.458 min, m/z=674.3 [M+H]⁺.

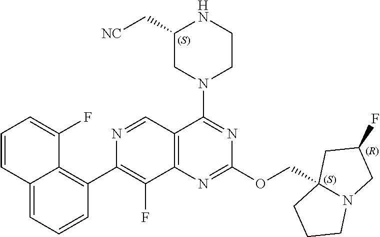

### Step 2: 2-((S)-4-(8-fluoro-7-(8-fluoronaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The Boc deprotection reaction was prepared in a similar fashion to Method #21, Step 2. The reaction mixture was concentrated in vacuo affording 2-((S)-4-(8-fluoro-7-(8-fluoronaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (1 g, crude, trifluoroacetate salt) as a brown oil, which was used in the next step without further purification. LCMS Rt=0.579 min, m/z=574.3 [M+H]⁺.

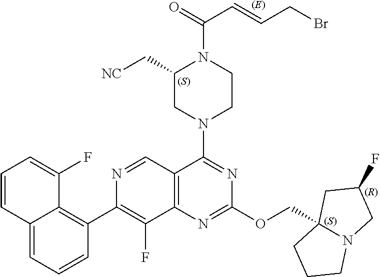

### Step 3: 2-((S)-1-((E)-4-bromobut-2-enoyl)-4-(8-fluoro-7-(8-fluoronaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The acylation reaction was prepared in a similar fashion to Method #21, Step 4. The resulting solution of 2-((S)-1-((E)-4-bromobut-2-enoyl)-4-(8-fluoro-7-(8-fluoronaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl) in acetonitrile (brown liquid) was used in the next step without any further purification. LCMS Rt=0.721 min, m/z=720.2 [M+H]⁺.

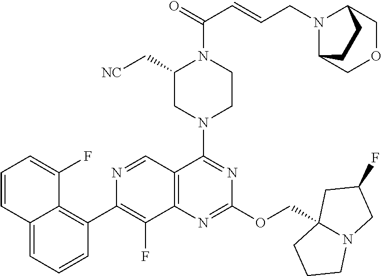

### Step 4: 2-((S)-1-((E)-4-((1R,5S)-3-oxa-8-azabicyclo[3.2.1]octan-8-yl)but-2-enoyl)-4-(8-fluoro-7-(8-fluoronaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The crude product was purified by reverse phase HPLC (column: Waters Xbridge Prep OBD C18 150*40 mm*10 um; mobile phase: [water (NH₄HCO₃)-ACN]; gradient: 30%-60% B over 8 min) affording 2-((S)-1-((E)-4-((1R,5S)-3-oxa-8-azabicyclo[3.2.1]octan-8-yl)but-2-enoyl)-4-(8-fluoro-7-(8-fluoronaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (224.32 mg, 42.12%) as a yellow amorphous solid: ¹H NMR (400 MHz, Acetonitrile-d3) δ 9.14 (d, J=2.5 Hz, 1H), 8.11 (br d, J=8.3 Hz, 1H), 7.87 (d, J=8.3 Hz, 1H), 7.73-7.67 (m, 1H), 7.62 (dd, J=7.1, 11.4 Hz, 1H), 7.54 (dt, J=5.1, 7.9 Hz, 1H), 7.22 (dd, J=7.6, 13.3 Hz, 1H), 6.83-6.55 (m, 2H), 5.35-5.16 (m, 1H), 5.06-4.69 (m, 1H), 4.60-4.34 (m, 3H), 4.24-4.10 (m, 2H), 4.06-3.77 (m, 2H), 3.73-3.58 (m, 3H), 3.45 (br d, J=9.8 Hz, 2H), 3.19-3.11 (m, 2H), 3.11-3.05 (m, 3H), 3.02 (br s, 2H), 2.95-2.83 (m, 3H), 2.24-2.15 (m, 2H), 2.10 (d, J=2.9 Hz, 1H), 2.08-2.01 (m, 1H), 1.92-1.88 (m, 2H), 1.88-1.83 (m, 2H), 1.83-1.76 (m, 2H).

LCMS (5% to 95% acetonitrile in water+0.03% ammonium bicarbonate over 6 min); retention time 2.900 min, ESI+found [M+H]=753.3.

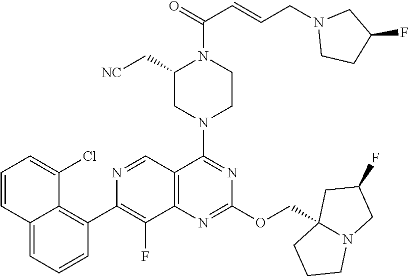 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@H](F)C1]

### Example 95 (Method 22): 2-((S)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)-1-((E)-4-((S)-3-fluoropyrrolidin-1-yl)but-2-enoyl)piperazin-2-yl)acetonitrile

The substitution reaction was prepared in a similar fashion to Method #21, Step 5. The reaction mixture was purified by reverse phase HPLC (column: Waters Xbridge Prep OBD C18 150*40 mm*10 um; mobile phase: [water (NH₄HCO₃)-ACN]; B %: 30%-60%, 8 min) affording 2—((S)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)-1-((E)-4-((S)-3-fluoropyrrolidin-1-yl)but-2-enoyl)piperazin-2-yl)acetonitrile (85.13 mg, 32.38%) as a yellow solid: ¹H NMR (400 MHz, Chloroform-d) δ 9.17-8.83 (m, 1H), 8.02-7.77 (m, 2H), 7.63-7.32 (m, 4H), 7.26-7.16 (m, 1H), 6.53-6.36 (m, 1H), 5.39-5.25 (m, 1H), 5.24-5.12 (m, 1H), 5.06 (br d, J=11.8 Hz, 1H), 4.59-4.32 (m, 3H), 4.31-4.11 (m, 2H), 4.06-3.85 (m, 2H), 3.83-3.63 (m, 2H), 3.37-3.06 (m, 5H), 2.99-2.80 (m, 3H), 2.73 (br d, J=1.3 Hz, 2H), 2.43 (br d, J=6.9 Hz, 1H), 2.28-2.01 (m, 5H), 1.97-1.77 (m, 3H).

LCMS (5% to 95% acetonitrile in water+0.03% ammonium bicarbonate over 6 min); retention time 2.988 min, ESI+found [M+H]=745.3.

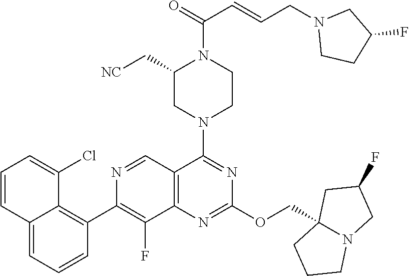 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@@H](F)C1]

### Example 96 (Method 22): 2-((S)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)-1-((E)-4-((R)-3-fluoropyrrolidin-1-yl)but-2-enoyl)piperazin-2-yl)acetonitrile

The substitution reaction was prepared in a similar fashion to Method #21, Step 5. The reaction mixture was purified by reverse phase HPLC (column: Waters Xbridge Prep OBD C18 150*40 mm*10 um; mobile phase: [water (NH₄HCO₃)-ACN]; B %: 35%-65%, 8 min) affording 2-((S)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)-1-((E)-4-((R)-3-fluoropyrrolidin-1-yl)but-2-enoyl)piperazin-2-yl)acetonitrile (91.72 mg, 30.24%) as a white solid: ¹H NMR (400 MHz, Acetonitrile-d3) δ 9.12 (br s, 1H), 8.13 (br d, J=7.6 Hz, 1H), 8.07-7.98 (m, 1H), 7.73-7.58 (m, 3H), 7.56-7.48 (m, 1H), 6.90-6.73 (m, 1H), 6.67-6.46 (m, 1H), 5.39-5.07 (m, 2H), 5.03-4.67 (m, 1H), 4.61-4.37 (m, 3H), 4.25-4.18 (m, 1H), 4.17-4.09 (m, 1H), 4.06-3.95 (m, 1H), 3.90-3.60 (m, 3H), 3.28 (br s, 2H), 3.19-3.11 (m, 2H), 3.06 (br s, 1H), 2.95-2.80 (m, 5H), 2.73-2.60 (m, 1H), 2.41-2.33 (m, 1H), 2.19-2.13 (m, 3H), 2.11 (br s, 1H), 2.05 (br d, J=7.3 Hz, 1H), 1.90-1.83 (m, 2H).

LCMS (5% to 95% acetonitrile in water+0.03% ammonium bicarbonate over 6 min); retention time 2.986 min, ESI+found [M+H]=745.3.

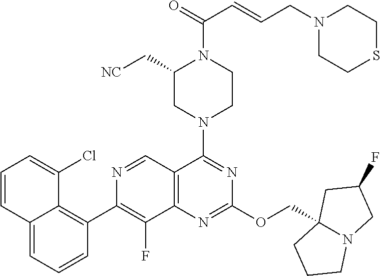 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCSCC1]

### Example 97 (Method 22): 2-((S)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)-1-((E)-4-thiomorpholinobut-2-enoyl)piperazin-2-yl)acetonitrile

The substitution reaction was prepared in a similar fashion to Method #21, Step 5. The reaction mixture was purified by reverse phase HPLC (column: Waters Xbridge Prep OBD C18 150*40 mm*10 um; mobile phase: [water (NH₄HCO₃)-ACN]; B %: 35%-65%, 8 min) affording 2-((S)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)-1-((E)-4-thiomorpholinobut-2-enoyl)piperazin-2-yl)acetonitrile (43.44 mg, 27.67%) as a yellow solid: ¹H NMR (400 MHz, Acetonitrile-d3) δ 9.19-9.10 (m, 1H), 8.16 (dd, J=1.1, 8.1 Hz, 1H), 8.05 (d, J=8.3 Hz, 1H), 7.71 (dd, J=1.8, 8.1 Hz, 1H), 7.68-7.61 (m, 2H), 7.57-7.51 (m, 1H), 6.84-6.50 (m, 2H), 5.39-5.18 (m, 1H), 5.13-4.72 (m, 1H), 4.59-4.40 (m, 2H), 4.28-4.22 (m, 1H), 4.19-4.12 (m, 1H), 4.08-3.88 (m, 1H), 3.86-3.61 (m, 2H), 3.19 (br d, J=5.1 Hz, 3H), 3.16-3.12 (m, 1H), 3.09 (s, 1H), 2.95-2.89 (m, 2H), 2.71 (br s, 3H), 2.70-2.67 (m, 3H), 2.21 (br d, J=4.1 Hz, 1H), 2.15-2.11 (m, 3H), 2.10-2.05 (m, 1H), 1.94-1.83 (m, 4H), 1.80 (td, J=2.4, 4.9 Hz, 1H).

LCMS (5% to 95% acetonitrile in water+0.03% ammonium bicarbonate over 6 min); retention time 3.055 min, ESI+found [M+H]=759.3.

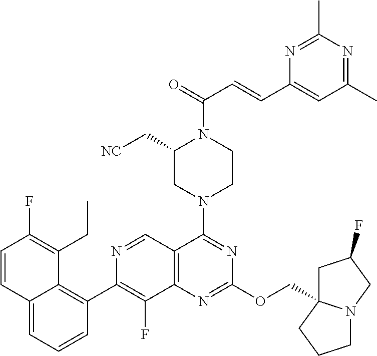 [SMILES: CCc1c(F)ccc2cccc(-c3ncc4c(N5CCN(C(=O)/C=C/c6cc(C)nc(C)n6)[C@@H](CC#N)C5)nc(OC[C@@]56CCCN5C[C@H](F)C6)nc4c3F)c12]

### Example 98 (Method 1): 2-((S)-1-((E)-3-(2,6-dimethylpyrimidin-4-yl)acryloyl)-4-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The amide coupling reaction was prepared in a similar fashion to Method #1, Step 4. The crude product was purified by reverse phase prep-HPLC (column: Welch Xtimate C18 250*70 mm*10 um; mobile phase: [water (NH₄HCO₃)-ACN]; B %: 40%-70%, 20 min) affording 2-((S)-1-((E)-3-(2,6-dimethylpyrimidin-4-yl)acryloyl)-4-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (319 mg, 55.89%) as a white solid: ¹H NMR (400 MHz, Acetonitrile -d3) δ 9.20-9.14 (m, 1H), 8.09 (dd, J=1.3, 8.0 Hz, 1H), 7.97 (dd, J=6.1, 9.0 Hz, 1H), 7.68 (br d, J=14.9 Hz, 1H), 7.62-7.38 (m, 4H), 7.26 (br s, 1H), 5.38-5.21 (m, 1H), 5.15-4.86 (m, 1H), 4.62-4.46 (m, 2H), 4.30-4.25 (m, 1H), 4.17 (br d, J=10.3 Hz, 2H), 4.02-3.64 (m, 3H), 3.22-3.14 (m, 2H), 3.13-3.08 (m, 1H), 3.02-2.88 (m, 3H), 2.64 (br s, 3H), 2.48 (s, 3H), 2.37-2.22 (m, 2H), 2.15-2.04 (m, 3H), 1.95-1.82 (m, 3H), 0.84 (q, J=7.2 Hz, 3H).

LCMS (5% to 95% acetonitrile in water+0.03% ammonium bicarbonate over 6 min); retention time 2.294 min, ESI+found [M+H]=762.3.

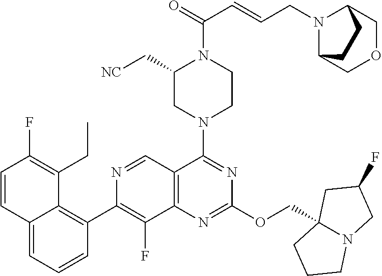

### Example 99 (Method 25): 2-((S)-1-((E)-4-((1R,5S)-3-oxa-8-azabicyclo[3.2.1]octan-8-yl)but-2-enoyl)-4-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

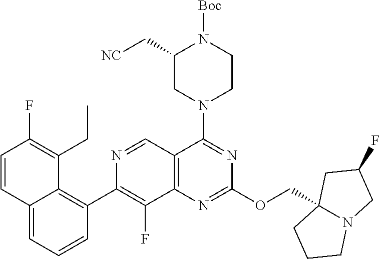 [SMILES: CCc1c(F)ccc2cccc(-c3ncc4c(N5CCN(C(=O)OC(C)(C)C)[C@@H](CC#N)C5)nc(OC[C@@]56CCCN5C[C@H](F)C6)nc4c3F)c12]

### Step 1: tert-butyl (S)-2-(cyanomethyl)-4-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazine-1-carboxylate

The Suzuki reaction was prepared in a similar fashion to Method #16, Step 7. The residue was purified by column chromatography (silica gel, 100-200 mesh, 0-10% methanol in dichloromethane) affording tert-butyl (S)-2-(cyanomethyl)-4-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazine-1-carboxylate (2.4 g, 96.45%) as a yellow oil. LCMS Rt=2.659 min, m/z=702.3 [M+H]⁺.

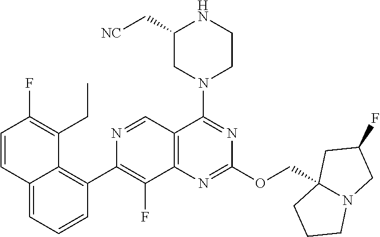 [SMILES: CCc1c(F)ccc2cccc(-c3ncc4c(N5CCN[C@@H](CC#N)C5)nc(OC[C@@]56CCCN5C[C@H](F)C6)nc4c3F)c12]

### Step 2: 2-((S)-4-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The Boc deprotection reaction was prepared in a similar fashion to Method #21, Step 2. The reaction mixture was concentrated in vacuo affording 2-((S)-4-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (400 mg, crude, trifluoroacetate acid) as a brown oil, which was used in the next step without further purification. LCMS Rt=0.661 min, m/z=602.3 [M+H]⁺.

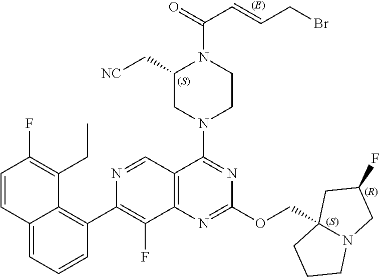

### Step 3: 2-((S)-1-((E)-4-bromobut-2-enoyl)-4-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The acylation reaction was prepared in a similar fashion to Method #21, Step 4. The resulting solution of 2-((S)-1-((E)-4-bromobut-2-enoyl)-4-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl) in acetonitrile (brown liquid) was used in the next step without any further purification. LCMS Rt=0.771 min, m/z=748.2 [M+H]⁺.

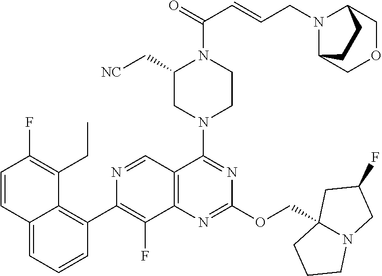

### Step 4: 2-((S)-1-((E)-4-((1R,5S)-3-oxa-8-azabicyclo[3.2.1]octan-8-yl)but-2-enoyl)-4-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The crude product was purified by reverse phase HPLC (column: Waters Xbridge Prep OBD C18 150*40 mm*10 um; mobile phase: [water (NH₄HCO₃)-ACN]; B %: 35%-65%, 8 min) affording 2-((S)-1-((E)-4-((1R,5S)-3-oxa-8-azabicyclo[3.2.1]octan-8-yl)but-2-enoyl)-4-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (40.49 mg, 36.61%) as a yellow amorphous solid: ¹H NMR (400 MHz, Dimethyl sulfoxide-d6) δ 9.22 (s, 1H), 8.14 (d, J=8.1 Hz, 1H), 8.04 (dd, J=6.2, 8.9 Hz, 1H), 7.60 (dt, J=4.4, 7.5 Hz, 1H), 7.54-7.45 (m, 2H), 6.82-6.63 (m, 2H), 5.39-5.18 (m, 1H), 4.99-4.81 (m, 1H), 4.58-4.27 (m, 3H), 4.23-3.99 (m, 3H), 3.92-3.69 (m, 2H), 3.57 (br d, J=9.5 Hz, 2H), 3.42 (br d, J=9.9 Hz, 2H), 3.19-2.96 (m, 9H), 2.88-2.79 (m, 1H), 2.45 (br d, J=4.3 Hz, 1H), 2.31-2.12 (m, 2H), 2.09-1.99 (m, 2H), 1.91-1.77 (m, 5H), 1.74 (br s, 2H), 0.82-0.71 (m, 3H).

LCMS (5% to 95% acetonitrile in water+0.03% ammonium bicarbonate over 6 min); retention time 2.053 min, ESI+found [M+H]=781.4.

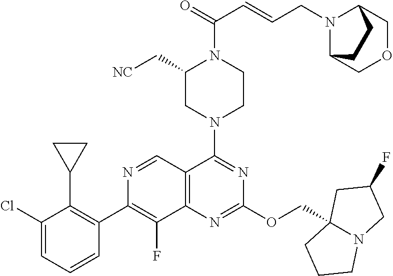

### Example 100(Method 26): 2-((S)-1-((E)-4-((1R,5S)-3-oxa-8-azabicyclo[3.2.1]octan-8-yl)but-2-enoyl)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

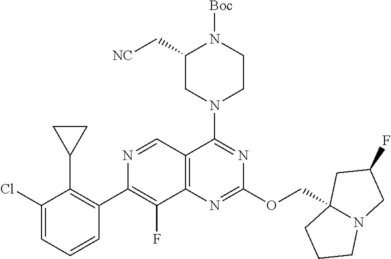 [SMILES: CC(C)(C)OC(=O)N1CCN(c2nc(OC[C@@]34CCCN3C[C@H](F)C4)nc3c(F)c(-c4cccc(Cl)c4C4CC4)ncc23)C[C@@H]1CC#N]

### Step 1: tert-butyl (S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate

The Suzuki reaction was prepared in a similar fashion to Method #16, Step 7. The residue was purified by column chromatography (silica gel, 100-200 mesh, 0-90% ethyl acetate in petroleum ether) affording tert-butyl (S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (1.76 g, 62.99%) as a pale yellow solid. LCMS Rt=0.663 min, m/z=680.3 [M+H]⁺.

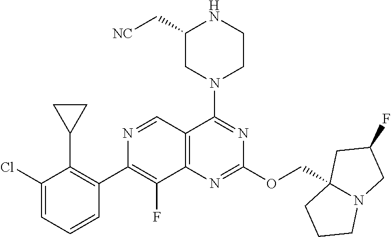 [SMILES: N#CC[C@H]1CN(c2nc(OC[C@@]34CCCN3C[C@H](F)C4)nc3c(F)c(-c4cccc(Cl)c4C4CC4)ncc23)CCN1]

### Step 2: 2-((S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The Boc deprotection reaction was prepared in a similar fashion to Method #21, Step 2. The mixture was concentrated to dryness in vacuo affording 2-((S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (300 mg, crude, trifluoroacetate acid) as a brown oil, which was used in the next step without any further purification. LCMS Rt=0.498 min, m/z=580.2 [M+H]⁺.

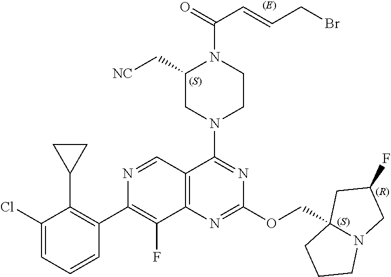

### Step 3: 2-((S)-1-((E)-4-bromobut-2-enoyl)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The acylation reaction was prepared in a similar fashion to Method #21, Step 4. The resulting solution of 2-((S)-1-((E)-4-bromobut-2-enoyl)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl) in acetonitrile (brown liquid) was used in the next step without any further purification. LCMS Rt=0.651 min, m/z=726.2 [M+H]⁺.

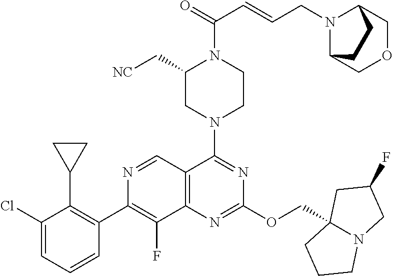

### Step 4: 2-((S)-1-((E)-4-((1R,5S)-3-oxa-8-azabicyclo[3.2.1]octan-8-yl)but-2-enoyl)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The residue was purified by reverse phase HPLC (column: Waters Xbridge Prep OBD C18 150*40 mm*10 um; mobile phase: [water (NH₄HCO₃)-ACN]; B %: 35%-65%, 8 min) affording 2-((S)-1-((E)-4-((1R,5S)-3-oxa-8-azabicyclo[3.2.1]octan-8-yl)but-2-enoyl)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (36.8 mg, 34.99%) as a yellow amorphous solid: ¹H NMR (400 MHz, Acetonitrile-d3) δ 9.02 (s, 1H), 7.44 (dd, J=1.4, 7.7 Hz, 1H), 7.35-7.21 (m, 2H), 6.76-6.43 (m, 2H), 5.26-5.02 (m, 1H), 4.96-4.60 (m, 1H), 4.52-4.22 (m, 3H), 4.16-4.08 (m, 1H), 4.06-3.99 (m, 1H), 3.88-3.58 (m, 3H), 3.52 (br d, J=10.3 Hz, 2H), 3.35 (br d, J=9.8 Hz, 2H), 3.07-3.01 (m, 2H), 3.00-2.95 (m, 3H), 2.92 (br d, J=1.0 Hz, 2H), 2.86-2.74 (m, 3H), 2.08 (br d, J=4.4 Hz, 2H), 2.00 (br d, J=2.5 Hz, 2H), 1.98-1.93 (m, 1H), 1.92-1.86 (m, 2H), 1.76 (br dd, J=4.3, 10.9 Hz, 2H), 1.72-1.68 (m, 2H), 0.70-0.52 (m, 2H), 0.09-0.07 (m, 2H).

LCMS (5% to 95% acetonitrile in water+0.03% ammonium bicarbonate over 6 min); retention time 3.100 min, ESI+found [M+H]=759.3.

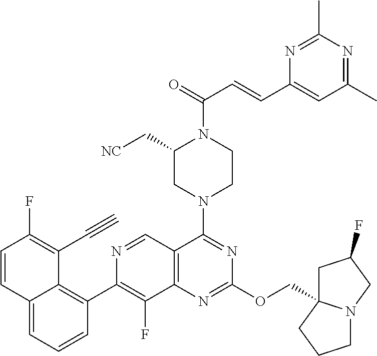 [SMILES: C#Cc1c(F)ccc2cccc(-c3ncc4c(N5CCN(C(=O)/C=C/c6cc(C)nc(C)n6)[C@@H](CC#N)C5)nc(OC[C@@]56CCCN5C[C@H](F)C6)nc4c3F)c12]

### Example 101 (Method 1): 2-((S)-1-((E)-3-(2,6-dimethylpyrimidin-4-yl)acryloyl)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

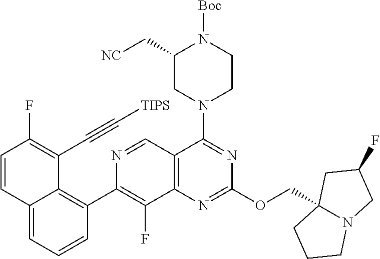

### Step 1: tert-butyl (S)-2-(cyanomethyl)-4-(8-fluoro-7-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)piperazine-1-carboxylate

The Suzuki reaction was prepared in a similar fashion to Method #16, Step 7. The residue was purified by column chromatography (silica gel, 100-200 mesh, 0-100% ethyl acetate in petroleum ether) affording tert-butyl (S)-2-(cyanomethyl)-4-(8-fluoro-7-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)piperazine-1-carboxylate (1.5 mg, 30%) as a brown solid. LCMS Rt=0.865 min, m/z=854.4 [M+H]⁺.

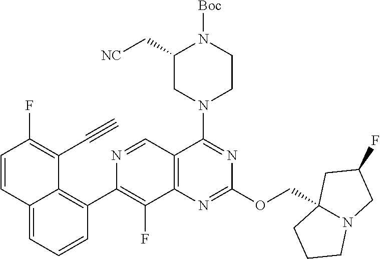 [SMILES: C#Cc1c(F)ccc2cccc(-c3ncc4c(N5CCN(C(=O)OC(C)(C)C)[C@@H](CC#N)C5)nc(OC[C@@]56CCCN5C[C@H](F)C6)nc4c3F)c12]

### Step 2: tert-butyl (S)-2-(cyanomethyl)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazine-1-carboxylate

To a solution of tert-butyl (S)-2-(cyanomethyl)-4-(8-fluoro-7-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)piperazine-1-carboxylate (400 mg, 468.34 umol) in N,N-dimethylformaldehyde (5 mL) was added cesium fluoride (711.42 mg, 4.68 mmol), and the mixture was stirred at 25° C. for 1 h. Water (10 mL) was added, and the mixture was extracted with ethyl acetate (3×20 mL). The combined organic layers were dried over sodium sulphate and concentrated in vacuo. The resulting residue was purified by column chromatography (silica gel, 100-200 mesh, 0-100% ethyl acetate in petroleum ether) affording tert-butyl (S)-2-(cyanomethyl)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazine-1-carboxylate (300 mg, 81.71%) as a yellow gum. LCMS Rt=0.641 min, m/z=698.3 [M+H]⁺.

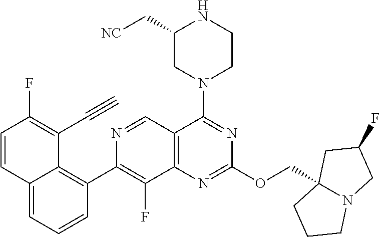 [SMILES: C#Cc1c(F)ccc2cccc(-c3ncc4c(N5CCN[C@@H](CC#N)C5)nc(OC[C@@]56CCCN5C[C@H](F)C6)nc4c3F)c12]

### Step 3: 2-((S)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The deprotection of the Boc group was prepared in a similar fashion to Method #21, Step 2. The reaction mixture was concentrated in vacuo affording 2-((S)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (240 mg, crude, hydrochloride salt) as a yellow gum, which was used in the next step without any further purification. LCMS Rt=0.529 min, m/z=598.3 [M+H]⁺.

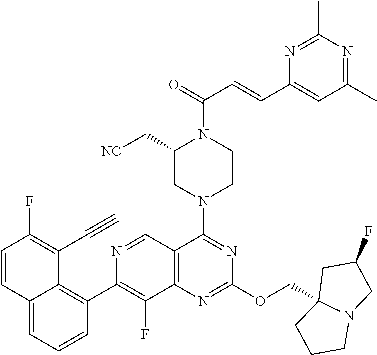 [SMILES: C#Cc1c(F)ccc2cccc(-c3ncc4c(N5CCN(C(=O)/C=C/c6cc(C)nc(C)n6)[C@@H](CC#N)C5)nc(OC[C@@]56CCCN5C[C@H](F)C6)nc4c3F)c12]

### Step 4: 2-((S)-1-((E)-3-(2,6-dimethylpyrimidin-4-yl)acryloyl)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The amide coupling reaction was prepared in a similar fashion to Method #1, Step 4. The crude product was purified by reverse phase prep-HPLC (column: Waters Xbridge Prep OBD C18 150*40 mm*10 um; mobile phase: [water (NH₄HCO₃)-ACN]; B %: 35%-65%, 8 min) affording 2-((S)-1-((E)-3-(2,6-dimethylpyrimidin-4-yl)acryloyl)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (157.91 mg, 52.59%) as a yellow solid: ¹H NMR (400 MHz, Chloroform-d) δ 9.14-9.03 (m, 1H), 8.06-7.90 (m, 2H), 7.76-7.67 (m, 1H), 7.66-7.50 (m, 3H), 7.40-7.31 (m, 1H), 7.04-6.95 (m, 1H), 5.44-5.21 (m, 1H), 5.18-4.86 (m, 1H), 4.61 (dt, J=3.7, 13.9 Hz, 1H), 4.54-4.44 (m, 1H), 4.43-4.19 (m, 3H), 4.18-3.79 (m, 3H), 3.44-3.20 (m, 3H), 3.09-2.80 (m, 4H), 2.73 (br s, 3H), 2.54 (s, 3H), 2.34-2.15 (m, 3H), 1.99 (br s, 3H).

LCMS (5% to 95% acetonitrile in water+0.03% ammonium bicarbonate over 6 min); retention time 2.954 min, ESI+found [M+H]=758.3.

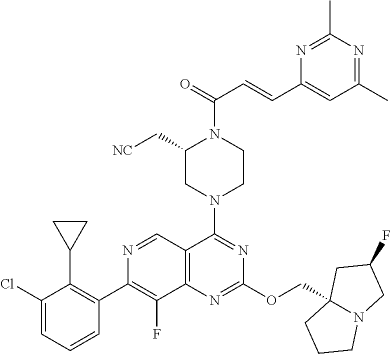 [SMILES: Cc1cc(/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)nc(C)n1]

### Example 102 (Method 1): 2-((S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2,6-dimethylpyrimidin-4-yl)acryloyl)piperazin-2-yl)acetonitrile

The amide coupling reaction was prepared in a similar fashion to Method #1, Step 4. The crude product was purified by reverse phase prep-HPLC (column: Phenomenex C18 80*40 mm*3 um; mobile phase: [water (NH₄HCO₃)-ACN]; B %: 35%-65%, 8 min) affording 2-((S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2,6-dimethylpyrimidin-4-yl)acryloyl)piperazin-2-yl)acetonitrile (153.97 mg, 28.87%) as a yellow solid: ¹H NMR (400 MHz, Chloroform-d) δ 9.13 (s, 1H), 7.78-7.71 (m, 1H), 7.62-7.55 (m, 1H), 7.54-7.47 (m, 1H), 7.42-7.38 (m, 1H), 7.34 (d, J=7.8 Hz, 1H), 7.02 (s, 1H), 5.49-5.22 (m, 1H), 5.14 (br d, J=4.0 Hz, 1H), 4.71-4.47 (m, 2H), 4.43-4.23 (m, 3H), 4.18-4.05 (m, 1H), 4.04-3.84 (m, 2H), 3.49-3.22 (m, 3H), 3.19-2.97 (m, 2H), 2.91-2.80 (m, 1H), 2.75 (br s, 3H), 2.57 (s, 3H), 2.35 (br s, 1H), 2.31-2.17 (m, 2H), 2.14-2.07 (m, 1H), 2.05-1.95 (m, 3H), 0.89-0.66 (m, 2H), 0.18 (br d, J=2.7 Hz, 2H).

LCMS (5% to 95% acetonitrile in water+0.03% ammonium bicarbonate over 6 min); retention time 3.128 min, ESI+found [M+H]=740.3.

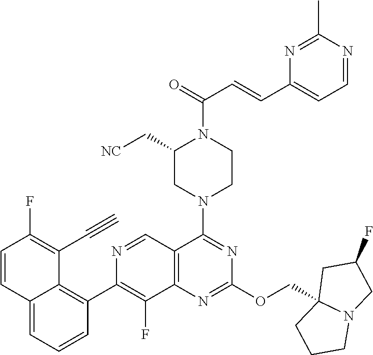 [SMILES: C#Cc1c(F)ccc2cccc(-c3ncc4c(N5CCN(C(=O)/C=C/c6ccnc(C)n6)[C@@H](CC#N)C5)nc(OC[C@@]56CCCN5C[C@H](F)C6)nc4c3F)c12]

### Example 103 (Method 1): 2-((S)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2-methylpyrimidin-4-yl)acryloyl)piperazin-2-yl)acetonitrile

The amide coupling reaction was prepared in a similar fashion to Method #1, Step 4. The crude product was purified by reverse phase prep-HPLC (column: Waters Xbridge BEH C18 250*50 mm*10 um; mobile phase: [water (NH₄HCO₃)-ACN]; B %: 40%-60%, 10 min) affording 2-((S)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2-methylpyrimidin-4-yl)acryloyl)piperazin-2-yl)acetonitrile (164.47 mg, 29.99%) as a yellow solid: ¹H NMR (400 MHz, Acetonitrile-d3) δ 9.14-9.08 (m, 1H), 8.70 (br d, J=4.5 Hz, 1H), 8.16-8.10 (m, 2H), 7.74-7.63 (m, 3H), 7.52-7.41 (m, 2H), 7.37 (br s, 1H), 5.38-5.18 (m, 1H), 5.13-4.88 (m, 1H), 4.48 (br d, J=13.1 Hz, 3H), 4.28-4.21 (m, 1H), 4.15 (br d, J=9.9 Hz, 1H), 4.03-3.88 (m, 1H), 3.85 (br s, 1H), 3.74-3.42 (m, 1H), 3.35-3.26 (m, 1H), 3.16 (br d, J=12.4 Hz, 2H), 3.12-3.07 (m, 1H), 3.05-2.86 (m, 3H), 2.68 (br s, 3H), 2.21 (br d, J=6.0 Hz, 1H), 2.13 (br s, 1H), 2.10-2.04 (m, 1H), 1.95-1.83 (m, 3H).

LCMS (5% to 95% acetonitrile in water+0.03% ammonium bicarbonate over 6 min); retention time 2.904 min, ESI+found [M+H]=744.3.

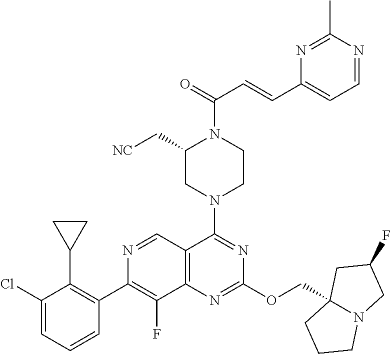 [SMILES: Cc1nccc(/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)n1]

### Example 104 (Method 1): 2-((S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2-methylpyrimidin-4-yl)acryloyl)piperazin-2-yl)acetonitrile

The amide coupling reaction was prepared in a similar fashion to Method #1, Step 4. The crude product was purified by reverse phase prep-HPLC (column: Waters Xbridge Prep OBD C18 150*40 mm*10 um; mobile phase: [water (NH₄HCO₃)-ACN]; B %: 35%-65%, 8 min) affording 2-((S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-3-(2-methylpyrimidin-4-yl)acryloyl)piperazin-2-yl)acetonitrile (85.65 mg, 40.78%) as a yellow solid: ¹H NMR (400 MHz, Chloroform-d) δ 9.10 (s, 1H), 8.72 (d, J=5.0 Hz, 1H), 7.78-7.71 (m, 1H), 7.63-7.56 (m, 1H), 7.50 (dd, J=1.1, 7.8 Hz, 1H), 7.40-7.37 (m, 1H), 7.32 (d, J=7.8 Hz, 1H), 7.14 (d, J=4.9 Hz, 1H), 5.38-5.22 (m, 1H), 5.12 (br d, J=3.1 Hz, 1H), 4.66-4.48 (m, 2H), 4.36-4.31 (m, 1H), 4.25 (br d, J=9.9 Hz, 2H), 4.13-3.70 (m, 3H), 3.26 (br d, J=9.3 Hz, 2H), 3.22-3.15 (m, 1H), 3.09-2.93 (m, 2H), 2.91-2.81 (m, 1H), 2.78 (s, 3H), 2.36-2.24 (m, 1H), 2.23-2.02 (m, 3H), 2.01-1.85 (m, 3H), 0.72 (br d, J=6.1 Hz, 2H), 0.16 (br s, 2H).

LCMS (5% to 95% acetonitrile in water+0.03% ammonium bicarbonate over 6 min); retention time 3.124 min, ESI+found [M+H]=726.3.

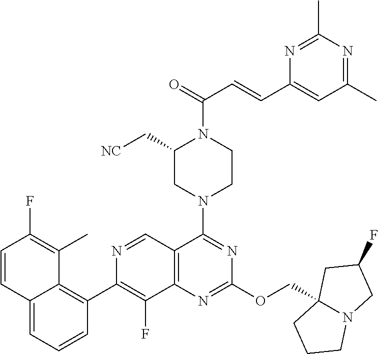 [SMILES: Cc1cc(/C=C/C(=O)N2CCN(c3nc(OC[C@@]45CCCN4C[C@H](F)C5)nc4c(F)c(-c5cccc6ccc(F)c(C)c56)ncc34)C[C@@H]2CC#N)nc(C)n1]

### Example 105 (Method 1): 2-((S)-1-((E)-3-(2,6-dimethylpyrimidin-4-yl)acryloyl)-4-(8-fluoro-7-(7-fluoro-8-methylnaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

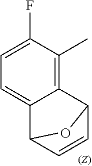

### Step 1: 4-fluoro-3-methyl-11-oxatricyclo[6.2.1.02,7]undeca-2,4,6,9-tetraene

To a solution of 1-bromo-2,4-difluoro-3-methyl-benzene (20 g, 96.61 mmol) and furan (13.15 g, 193.22 mmol) in toluene (300 mL) was added n-butyllithium (2.5 M, 46.37 mL) at −20° C. The mixture was stirred at 20° C. for 12 h under a nitrogen atmosphere. The reaction mixture was quenched with saturated ammonium chloride (900 mL) and extracted with dichloromethane (3×500 mL). The combined organic layers were dried over sodium sulphate and concentrated in vacuo. The residue was purified by column chromatography (silica gel, 100-200 mesh, 0-10% ethyl acetate in petroleum ether) affording 4-fluoro-3-methyl-11-oxatricyclo[6.2.1.02,7]undeca-2,4,6,9-tetraene (30 g, 58.75%) as a yellow oil. LCMS Rt=0.676 min, m/z=177.1 [M+H]⁺.

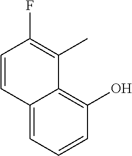

### Step 2: 7-fluoro-8-methyl-naphthalen-1-ol

To a solution of 4-fluoro-3-methyl-11-oxatricyclo[6.2.1.02,7]undeca-2,4,6,9-tetraene (30 g, 170.27 mmol) in ethanol (300 mL) was added hydrochloric acid (12 M, 170.27 mL). The mixture was stirred at 80° C. for 2 h and then concentrated in vacuo. The residue was purified by column chromatography (silica gel, 100-200 mesh, 0-10% ethyl acetate in petroleum ether) affording 7-fluoro-8-methyl-naphthalen-1-ol (16.2 g, 54.00%) as a red solid. LCMS Rt=0.732 min, m/z=177.1 [M+H]⁺.

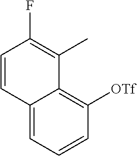 [SMILES: Cc1c(F)ccc2cccc(OS(=O)(=O)C(F)(F)F)c12]

### Step 3: (7-fluoro-8-methyl-1-naphthyl) trifluoromethanesulfonate

To a mixture of 7-fluoro-8-methyl-naphthalen-1-ol (5 g, 28.38 mmol) in dichloromethane (50 mL) was added N,N-diisopropylethylamine (22.01 g, 170.27 mmol) and trifluoromethanesulfonic anhydride (10.41 g, 36.89 mmol). The mixture was stirred at 0° C. for 0.5 h under a nitrogen atmosphere. The reaction mixture was quenched with saturated sodium bicarbonate (50 mL) and extracted with dichloromethane (3×50 mL). The combined organic layers were dried over sodium sulphate and concentrated in vacuo. The residue was purified by column chromatography (silica gel, 100-200 mesh, 0-10% ethyl acetate in petroleum ether) affording (7-fluoro-8-methyl-1-naphthyl) trifluoromethanesulfonate (8 g, 91.45%) as a yellow oil. LCMS Rt=0.951 min, m/z=309.0 [M+H]⁺.

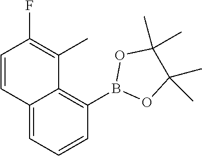 [SMILES: Cc1c(F)ccc2cccc(B3OC(C)(C)C(C)(C)O3)c12]

### Step 4: 2-(7-fluoro-8-methyl-1-naphthyl)-4,4,5,5-tetramethyl-1,3,2-dioxaborolane

A mixture of (7-fluoro-8-methyl-1-naphthyl) trifluoromethanesulfonate (25 g, 81.10 mmol), 4,4,5,5-tetramethyl-2-(4,4,5,5-tetramethyl-1,3,2-dioxaborolan-2-yl)-1,3,2-dioxaborolane (41.19 g, 162.21 mmol), potassium acetate (39.80 g, 405.52 mmol) and [1,1-Bis(diphenylphosphino)ferrocene]dichloropalladium(II) (5.93 g, 8.11 mmol) in dioxane (400 mL) was degassed and purged with nitrogen 3 times. The mixture was stirred at 80° C. for 12 h under a nitrogen atmosphere. The mixture was diluted with water (500 mL) and extracted with ethyl acetate (3×500 mL). The combined organic layers were dried over sodium sulphate and concentrated in vacuo. The residue was purified by column chromatography (silica gel, 100-200 mesh, 0-10% ethyl acetate in petroleum ether) affording 2-(7-fluoro-8-methyl-1-naphthyl)-4,4,5,5-tetramethyl-1,3,2-dioxaborolane (23 g, 99.11%) as a yellow solid. LCMS Rt=0.962 min, m/z=287.2 [M+H]⁺.

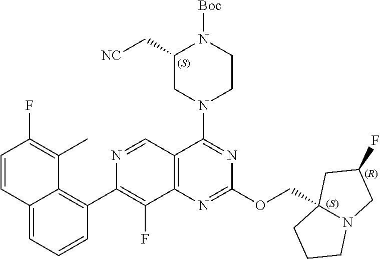

### Step 5: tert-butyl (S)-2-(cyanomethyl)-4-(8-fluoro-7-(7-fluoro-8-methylnaphthalen-1-yl)-2-(((2R,7aS)-2-fluorotetrahydro-1H-pyrrolizin-7a(5H)-yl)methoxy)pyrido[4,3-d]pyrimidin-4-yl)piperazine-1-carboxylate

The Suzuki coupling reaction was prepared in a similar fashion to Method #16, Step 7. The crude product was purified by column chromatography (silica gel, 100-200 mesh, 0-100% ethyl acetate in petroleum ether) affording tert-butyl (S)-2-(cyanomethyl)-4-(8-fluoro-7-(7-fluoro-8-methylnaphthalen-1-yl)-2-(((2R,7aS)-2-fluorotetrahydro-1H-pyrrolizin-7a(5H)-yl)methoxy)pyrido[4,3-d]pyrimidin-4-yl)piperazine-1-carboxylate (650 mg, 26.65%) as a black solid. LCMS Rt=0.805 min, m/z=688.3 [M+H]⁺.

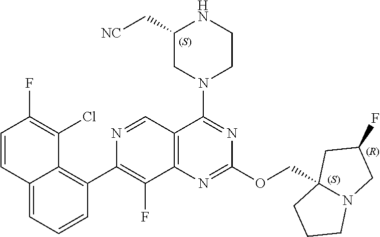

### Step 6: 2-((S)-4-(8-fluoro-7-(7-fluoro-8-methylnaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The Boc deprotection reaction was prepared in a similar fashion to Method #21, Step 2. The reaction mixture was concentrated in vacuo affording 2-((S)-4-(8-fluoro-7-(7-fluoro-8-methylnaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (100 mg, crude, trifluoroacetate salt) as a brown oil, which was used in the next step without further purification. LCMS Rt=0.635 min, m/z=588.3 [M+H]⁺.

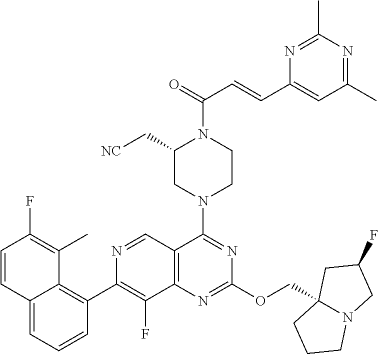 [SMILES: Cc1cc(/C=C/C(=O)N2CCN(c3nc(OC[C@@]45CCCN4C[C@H](F)C5)nc4c(F)c(-c5cccc6ccc(F)c(C)c56)ncc34)C[C@@H]2CC#N)nc(C)n1]

### Step 7: 2-((S)-1-((E)-3-(2,6-dimethylpyrimidin-4-yl)acryloyl)-4-(8-fluoro-7-(7-fluoro-8-methylnaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The amide coupling reaction was prepared in a similar fashion to Method #1, Step 4. The crude product was purified by reverse phase prep-HPLC (column: Waters Xbridge Prep OBD C18 150*40 mm*10 um; mobile phase: [water (NH₄HCO₃)-ACN]; B %: 35%-65%, 8 min) affording 2—((S)-1-((E)-3-(2,6-dimethylpyrimidin-4-yl)acryloyl)-4-(8-fluoro-7-(7-fluoro-8-methylnaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (27.64 mg, 25.89%) as a brown solid: ¹H NMR (400 MHz, Acetonitrile-d3) δ 9.14 (br s, 1H), 8.11-7.98 (m, 1H), 7.92 (dd, J=6.0, 8.9 Hz, 1H), 7.65 (br d, J=14.9 Hz, 1H), 7.60-7.52 (m, 2H), 7.45-7.35 (m, 2H), 7.28-7.17 (m, 1H), 5.40-5.16 (m, 1H), 5.11-4.81 (m, 1H), 4.58-4.41 (m, 2H), 4.27-4.20 (m, 1H), 4.13 (br d, J=10.3 Hz, 2H), 3.84 (br t, J=8.8 Hz, 3H), 3.18-3.11 (m, 2H), 3.06 (s, 1H), 2.99-2.80 (m, 3H), 2.61 (br s, 3H), 2.45 (s, 3H), 2.19 (br s, 1H), 2.10 (br s, 1H), 2.08-2.01 (m, 1H), 1.92-1.79 (m, 6H).

LCMS (5% to 95% acetonitrile in water+0.1% trifluoroacetic acid over 6 min); retention time 3.055 min, ESI+found [M+H]=748.3.

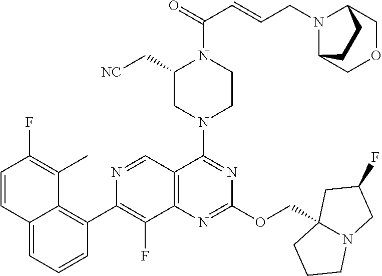

### Example 106 (Method 27): 2-((S)-1-((E)-4-((1R,5S)-3-oxa-8-azabicyclo[3.2.1]octan-8-yl)but-2-enoyl)-4-(8-fluoro-7-(7-fluoro-8-methylnaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

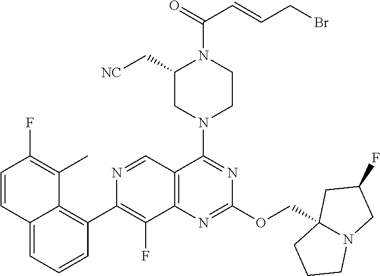 [SMILES: Cc1c(F)ccc2cccc(-c3ncc4c(N5CCN(C(=O)/C=C/CBr)[C@@H](CC#N)C5)nc(OC[C@@]56CCCN5C[C@H](F)C6)nc4c3F)c12]

### Step 1: 2-((S)-1-((E)-4-bromobut-2-enoyl)-4-(8-fluoro-7-(7-fluoro-8-methylnaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The amide coupling reaction was prepared in a similar fashion to Method #21, Step 4. The reaction was concentrated in vacuo affording 2-((S)-1-((E)-4-bromobut-2-enoyl)-4-(8-fluoro-7-(7-fluoro-8-methylnaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (125 mg, crude) as a yellow oil, which was used in the next step without further purification. LCMS Rt=0.766 min, m/z=736.2 [M+H]⁺.

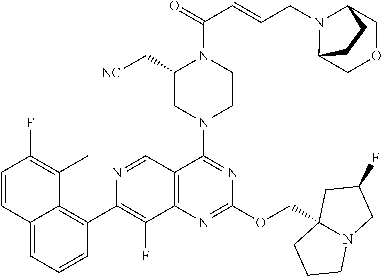

### Step 2: 2-((S)-1-((E)-4-((1R,5S)-3-oxa-8-azabicyclo[3.2.1]octan-8-yl)but-2-enoyl)-4-(8-fluoro-7-(7-fluoro-8-methylnaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The reaction mixture was purified by reverse phase HPLC (column: Waters Xbridge Prep OBD C18 150*40 mm*10 um; mobile phase: [water (NH₄HCO₃)-ACN]; B %: 30%-60%, 8 min) affording 2-((S)-1-((E)-4-((1R,5S)-3-oxa-8-azabicyclo[3.2.1]octan-8-yl)but-2-enoyl)-4-(8-fluoro-7-(7-fluoro-8-methylnaphthalen-1-yl)-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (15.68 mg, 12.02%) as a yellow amorphous solid: ¹H NMR (400 MHz, Acetonitrile-d3) δ 9.14 (br s, 1H), 8.10-7.87 (m, 2H), 7.63-7.50 (m, 2H), 7.47-7.30 (m, 1H), 6.92-6.41 (m, 2H), 5.40-5.15 (m, 1H), 4.57-4.39 (m, 2H), 4.31-4.18 (m, 1H), 4.17-4.08 (m, 1H), 3.90-3.70 (m, 2H), 3.62 (br d, J=9.9 Hz, 3H), 3.45 (br d, J=9.9 Hz, 2H), 3.14 (br d, J=8.3 Hz, 2H), 3.08 (br d, J=6.6 Hz, 3H), 3.02 (br s, 2H), 2.94-2.84 (m, 3H), 2.28-2.18 (m, 3H), 2.13-2.00 (m, 3H), 1.92-1.83 (m, 7H), 1.83-1.76 (m, 2H).

LCMS (5% to 95% acetonitrile in water+0.03% ammonium bicarbonate over 6 min); retention time 3.040 min, ESI+found [M+H]=767.4.

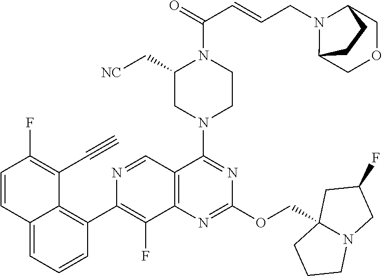

### Example 107 (Method 28): 2-((S)-1-((E)-4-((1R,5S)-3-oxa-8-azabicyclo[3.2.1]octan-8-yl)but-2-enoyl)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The crude product was purified by reverse phase HPLC (column: Waters Xbridge Prep OBD C18 150*40 mm*10 um; mobile phase: [water (NH₄HCO₃)-ACN]; B %: 30%-60%, 8 min) affording 2-((S)-1-((E)-4-((1R,5S)-3-oxa-8-azabicyclo[3.2.1]octan-8-yl)but-2-enoyl)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (17.44 mg, 24.05%) as a yellow amorphous solid: ¹H NMR (400 MHz, Chloroform-d) δ 9.11-9.02 (m, 1H), 8.03-7.92 (m, 2H), 7.68-7.56 (m, 2H), 7.35 (t, J=8.8 Hz, 1H), 7.06-6.95 (m, 1H), 6.64-6.49 (m, 1H), 5.38-5.21 (m, 1H), 5.13-4.88 (m, 1H), 4.61-4.43 (m, 2H), 4.37-4.29 (m, 1H), 4.29-4.20 (m, 1H), 4.19-3.97 (m, 2H), 3.93-3.83 (m, 1H), 3.81-3.70 (m, 3H), 3.59-3.52 (m, 2H), 3.26 (br d, J=9.9 Hz, 2H), 3.20-3.11 (m, 3H), 3.08-2.96 (m, 4H), 2.92-2.76 (m, 2H), 2.31-2.13 (m, 3H), 2.03 (br s, 1H), 1.96-1.89 (m, 6H).

LCMS (5% to 95% acetonitrile in water+0.03% ammonium bicarbonate over 6 min); retention time 2.936 min, ESI+found [M+H]=777.3.

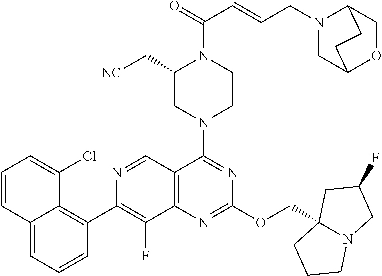 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CCC1CO2]

### Example 108 (Method 22): 2-((2S)-1-((E)-4-(2-oxa-5-azabicyclo[2.2.2]octan-5-yl)but-2-enoyl)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile

The substitution reaction was prepared in a similar fashion to Method #21, Step 5. The reaction mixture was purified by reverse phase HPLC (column: Waters Xbridge Prep OBD C18 150*40 mm*10 um; phase: [water (NH₄HCO₃)-ACN]; B %: 30%-60%, 8 min) affording 2-((2S)-1-((E)-4-(2-oxa-5-azabicyclo[2.2.2]octan-5-yl)but-2-enoyl)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile (151.26 mg, 28.98%) as a white solid: ¹H NMR (400 MHz, Acetonitrile-d3) δ 9.25-9.05 (m, 1H), 8.23-7.96 (m, 2H), 7.71 (dd, J=1.9, 8.1 Hz, 1H), 7.67-7.62 (m, 2H), 7.54 (dt, J=1.7, 7.8 Hz, 1H), 6.85-6.53 (m, 2H), 5.42-5.18 (m, 1H), 5.11-4.66 (m, 1H), 4.61-4.38 (m, 2H), 4.27-4.13 (m, 3H), 4.12-3.47 (m, 6H), 3.41 (br s, 2H), 3.22-3.12 (m, 2H), 3.11-2.85 (m, 6H), 2.64 (br s, 1H), 2.21 (br d, J=4.5 Hz, 1H), 2.13 (d, J=3.0 Hz, 2H), 2.11-1.99 (m, 2H), 1.95-1.84 (m, 3H), 1.79-1.66 (m, 2H).

LCMS (5% to 95% acetonitrile in water+0.03% ammonium bicarbonate over 6 min); retention time 2.919 min, ESI+found [M+H]=769.3.

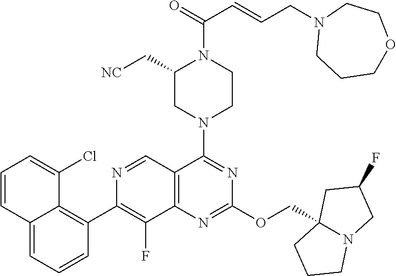 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCCOCC1]

### Example 109 (Method 22): 2-((S)-1-((E)-4-(1,4-oxazepan-4-yl)but-2-enoyl)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile

The substitution reaction was prepared in a similar fashion to Method #21, Step 5. The reaction mixture was purified by reverse phase HPLC (column: Waters Xbridge BEH C18 250*50 mm*10 um; mobile phase: [water (NH₄HCO₃)-ACN]; B %: 35%-65%, 10 min) affording 2-((S)-1-((E)-4-(1,4-oxazepan-4-yl)but-2-enoyl)-4-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile (205.04 mg, 39.31%) as a white solid: ¹H NMR (400 MHz, Acetonitrile -d3) δ 9.29-9.11 (m, 1H), 8.22-8.13 (m, 1H), 8.06 (d, J=8.2 Hz, 1H), 7.72 (dd, J=2.0, 8.1 Hz, 1H), 7.69-7.63 (m, 2H), 7.59-7.52 (m, 1H), 6.90-6.52 (m, 2H), 5.41-5.21 (m, 1H), 5.07-4.44 (m, 3H), 4.30-4.23 (m, 1H), 4.21-4.12 (m, 1H), 4.10-3.73 (m, 5H), 3.73-3.48 (m, 3H), 3.34 (br d, J=5.6 Hz, 2H), 3.24-3.13 (m, 2H), 3.10 (s, 1H), 3.00-2.84 (m, 3H), 2.76-2.66 (m, 4H), 2.28-2.21 (m, 2H), 2.14 (d, J=2.9 Hz, 1H), 2.12-2.04 (m, 1H), 1.96-1.92 (m, 1H), 1.92-1.87 (m, 3H).

LCMS (5% to 95% acetonitrile in water+0.03% ammonium bicarbonate over 6 min); retention time 1.969 min, ESI+found [M+H]=757.3.

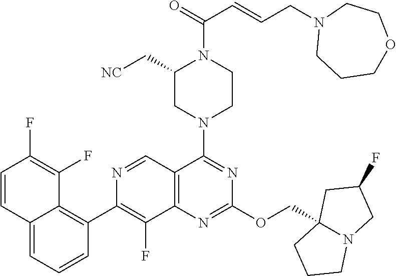 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCCOCC1]

### Example 110 (Method 23): 2-((S)-1-((E)-4-(1,4-oxazepan-4-yl)but-2-enoyl)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The residue was purified by reverse phase HPLC (column: Phenomenex Luna C18 200*40 mm*10 um; mobile phase: [water(FA)-ACN]; B %: 15%-45%, 8 min) affording 2-((S)-1-((E)-4-(1,4-oxazepan-4-yl)but-2-enoyl)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (50.85 mg, 9.26%, formate salt) as a yellow solid: ¹H NMR (400 MHz, Chloroform-d) δ 9.11-9.01 (m, 1H), 8.02-7.95 (m, 1H), 7.77-7.69 (m, 1H), 7.67-7.58 (m, 2H), 7.45-7.36 (m, 1H), 7.08-6.94 (m, 1H), 6.61-6.36 (m, 1H), 5.44-5.21 (m, 1H), 5.12-4.76 (m, 1H), 4.74-4.56 (m, 1H), 4.56-4.41 (m, 2H), 4.41-4.29 (m, 2H), 4.13-4.00 (m, 1H), 3.89-3.80 (m, 3H), 3.78-3.73 (m, 2H), 3.37 (br d, J=5.0 Hz, 4H), 3.32-3.16 (m, 2H), 3.10-2.96 (m, 2H), 2.83 (br s, 1H), 2.79-2.68 (m, 4H), 2.34 (br s, 1H), 2.27 (br s, 1H), 2.21 (br d, J=4.1 Hz, 1H), 2.05-1.97 (m, 3H), 1.97-1.92 (m, 2H).

LCMS (5% to 95% acetonitrile in water+0.03% ammonium bicarbonate over 6 min); retention time 2.916 min, ESI+found [M+H]=759.3.

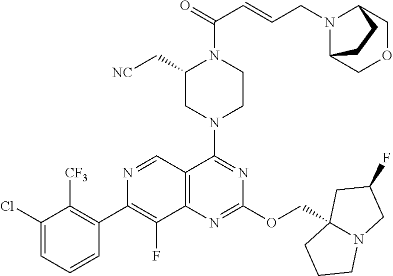

### Example 111 (Method 29): 2-((S)-1-((E)-4-((1R,5S)-3-oxa-8-azabicyclo[3.2.1]octan-8-yl)but-2-enoyl)-4-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

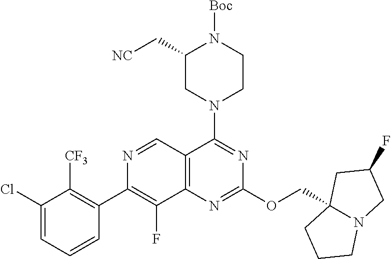 [SMILES: CC(C)(C)OC(=O)N1CCN(c2nc(OC[C@@]34CCCN3C[C@H](F)C4)nc3c(F)c(-c4cccc(Cl)c4C(F)(F)F)ncc23)C[C@@H]1CC#N]

### Step 1: tert-butyl (S)-4-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate

The Suzuki reaction was prepared in a similar fashion to Method #16, Step 7. The residue was purified by column chromatography (silica gel, 100-200 mesh, 0-100% methanol in dichloromethane) affording tert-butyl (S)-4-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (300 mg, 74.67%) as a yellow oil. LCMS Rt=0.798 min, m/z=708.2 [M+H]⁺.

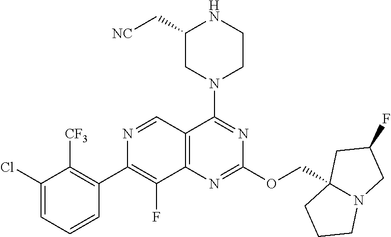 [SMILES: N#CC[C@H]1CN(c2nc(OC[C@@]34CCCN3C[C@H](F)C4)nc3c(F)c(-c4cccc(Cl)c4C(F)(F)F)ncc23)CCN1]

### Step 2: 2-((S)-4-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The Boc deprotection reaction was prepared in a similar fashion to Method #21, Step 2. The reaction mixture was concentrated in vacuo affording 2-((S)-4-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (254 mg, crude, trifluoroacetate acid) as a yellow oil, which was used in the next step without further purification. LCMS Rt=0.637 min, m/z=608.2 [M+H]⁺.

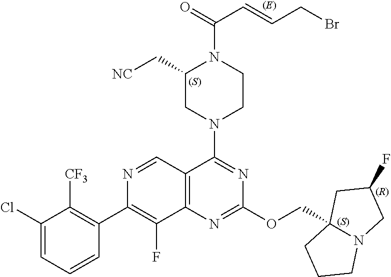

### Step 3: 2-((S)-1-((E)-4-bromobut-2-enoyl)-4-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The acylation reaction was prepared in a similar fashion to Method #21, Step 4. The resulting solution of 2-((S)-1-((E)-4-bromobut-2-enoyl)-4-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl) in acetonitrile (yellow liquid) was used in the next step without further purification.

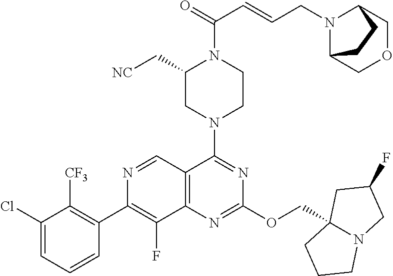

### Step 4: 2-((S)-1-((E)-4-((1R,5S)-3-oxa-8-azabicyclo[3.2.1]octan-8-yl)but-2-enoyl)-4-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The crude product was purified by reverse phase HPLC (column: Waters Xbridge Prep OBD C18 150*40 mm*10 um; mobile phase: [water (NH₄HCO₃)-ACN]; B %: 35%-65%, 8 min) affording 2-((S)-1-((E)-4-((1R,5S)-3-oxa-8-azabicyclo[3.2.1]octan-8-yl)but-2-enoyl)-4-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (74.34 mg, 22.29%) as a yellow amorphous solid: ¹H NMR (400 MHz, Acetonitrile -d3) δ 9.12 (s, 1H), 7.83-7.78 (m, 1H), 7.76-7.70 (m, 1H), 7.48 (br d, J=7.1 Hz, 1H), 6.81 (td, J=5.2, 14.9 Hz, 1H), 6.72-6.57 (m, 1H), 5.43-5.17 (m, 1H), 5.06-4.67 (m, 1H), 4.53-4.38 (m, 2H), 4.30-4.21 (m, 1H), 4.19-4.11 (m, 1H), 4.09-3.96 (m, 1H), 3.79 (br dd, J=3.4, 6.3 Hz, 2H), 3.71-3.54 (m, 3H), 3.48 (br d, J=9.1 Hz, 2H), 3.19-3.14 (m, 2H), 3.13-3.08 (m, 3H), 3.05 (br s, 2H), 2.98-2.86 (m, 3H), 2.13 (d, J=2.9 Hz, 1H), 2.08 (br s, 1H), 1.99-1.95 (m, 4H), 1.94-1.90 (m, 2H), 1.88 (br d, J=6.6 Hz, 1H), 1.83 (br d, J=7.5 Hz, 1H).

LCMS (5% to 95% acetonitrile in water+0.03% ammonium bicarbonate over 6 min); retention time 3.061 min, ESI+found [M+H]=787.3.

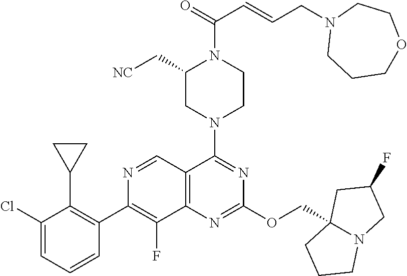 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CCCOCC1]

### Example 112 (Method 26): 2-((S)-1-((E)-4-(1,4-oxazepan-4-yl)but-2-enoyl)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The residue was purified by reverse phase HPLC (column: Waters Xbridge Prep OBD C18 150*40 mm*10 um; mobile phase: [water (NH₄HCO₃)-ACN]; B %: 35%-65%, 8 min) affording 2-((S)-1-((E)-4-(1,4-oxazepan-4-yl)but-2-enoyl)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (31.45 mg, 15.17%) as yellow solid: ¹H NMR (400 MHz, Chloroform -d) δ 9.09 (s, 1H), 7.49 (dd, J=1.1, 7.8 Hz, 1H), 7.40-7.35 (m, 1H), 7.31 (br d, J=7.8 Hz, 1H), 7.01 (td, J=5.5, 15.1 Hz, 1H), 6.48 (br d, J=14.5 Hz, 1H), 5.41-5.20 (m, 1H), 5.11-4.92 (m, 1H), 4.57-4.43 (m, 2H), 4.36-4.25 (m, 2H), 4.17-3.94 (m, 2H), 3.82 (br t, J=6.1 Hz, 3H), 3.77-3.72 (m, 2H), 3.38-3.18 (m, 5H), 3.04-2.95 (m, 2H), 2.81 (br s, 1H), 2.78-2.70 (m, 4H), 2.30 (br s, 1H), 2.25-2.13 (m, 2H), 2.09-1.88 (m, 7H), 0.72 (br s, 2H), 0.22-0.09 (m, 2H).

LCMS (5% to 95% acetonitrile in water+0.03% ammonium bicarbonate over 6 min); retention time 3.069 min, ESI+found [M+H]=746.3.

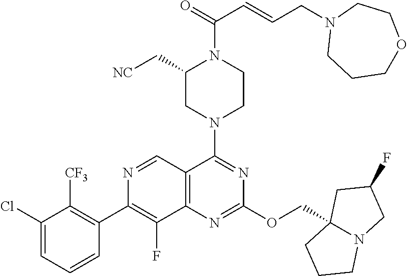 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CCCOCC1]

### Example 113 (Method 29): 2-((S)-1-((E)-4-(1,4-oxazepan-4-yl)but-2-enoyl)-4-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The substitution reaction was prepared in a similar fashion to Method #21, Step 5. The crude product was purified by reverse phase HPLC (column: Waters Xbridge Prep OBD C18 150*40 mm*10 um; mobile phase: [water (NH₄HCO₃)-ACN]; B %: 25%-55%, 8 min) affording 2-((S)-1-((E)-4-(1,4-oxazepan-4-yl)but-2-enoyl)-4-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (25.96 mg, 18.06%) as a yellow solid: ¹H NMR (400 MHz, Chloroform-d) δ 9.04 (s, 1H), 7.70-7.65 (m, 1H), 7.57 (t, J=7.9 Hz, 1H), 7.34 (d, J=7.6 Hz, 1H), 7.07-6.96 (m, 1H), 6.54-6.40 (m, 1H), 5.45-5.23 (m, 1H), 5.02 (br d, J=1.4 Hz, 1H), 4.63-4.33 (m, 4H), 4.14-3.94 (m, 2H), 3.92-3.79 (m, 3H), 3.77-3.74 (m, 2H), 3.36 (br d, J=4.6 Hz, 3H), 3.31-3.19 (m, 2H), 3.04 (br s, 2H), 2.83-2.70 (m, 5H), 2.38-2.15 (m, 3H), 2.08-1.89 (m, 6H).

LCMS (5% to 95% acetonitrile in water+0.03% ammonium bicarbonate over 6 min); retention time 2.982 min, ESI+found [M+H]=775.3.

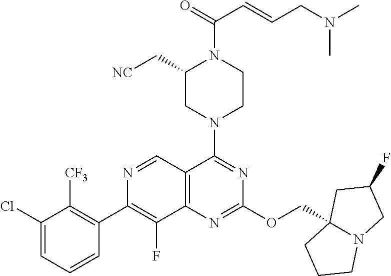 [SMILES: CN(C)C/C=C/C(=O)N1CCN(c2nc(OC[C@@]34CCCN3C[C@H](F)C4)nc3c(F)c(-c4cccc(Cl)c4C(F)(F)F)ncc23)C[C@@H]1CC#N]

### Example 114 (Method 29): 2-((S)-4-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-(dimethylamino)but-2-enoyl)piperazin-2-yl)acetonitrile

The substitution reaction was prepared in a similar fashion to Method #21, Step 5. The residue was purified by reverse phase HPLC (column: Waters Xbridge BEH C18 100*30 mm*10 um; mobile phase: [water (NH₄HCO₃)-ACN]; B %: 35%-65%, 8 min) affording 2-((S)-4-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-(dimethylamino)but-2-enoyl)piperazin-2-yl)acetonitrile (37.07 mg, 24.86%) as a yellow amorphous solid: ¹H NMR (400 MHz, Dimethylsulfoxide-d6) δ 9.18 (s, 1H), 7.96-7.90 (m, 1H), 7.84 (t, J=7.9 Hz, 1H), 7.54 (br d, J=7.6 Hz, 1H), 6.76-6.57 (m, 2H), 5.37-5.19 (m, 1H), 4.96-4.80 (m, 1H), 4.65-4.22 (m, 3H), 4.19-4.13 (m, 1H), 4.13-3.97 (m, 2H), 3.82 (br d, J=9.4 Hz, 2H), 3.71-3.55 (m, 1H), 3.10-2.99 (m, 6H), 2.89-2.78 (m, 1H), 2.17 (s, 6H), 2.13 (br d, J=3.8 Hz, 1H), 2.08-1.96 (m, 2H), 1.89-1.69 (m, 3H).

LCMS (5% to 95% acetonitrile in water+0.03% ammonium bicarbonate over 6 min); retention time 2.785 min, ESI+found [M+H]=719.3.

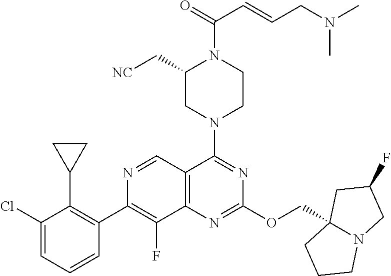 [SMILES: CN(C)C/C=C/C(=O)N1CCN(c2nc(OC[C@@]34CCCN3C[C@H](F)C4)nc3c(F)c(-c4cccc(Cl)c4C4CC4)ncc23)C[C@@H]1CC#N]

### Example 115 (Method 26): 2-((S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-(dimethylamino)but-2-enoyl)piperazin-2-yl)acetonitrile

The substitution reaction was prepared in a similar fashion to Method #21, Step 5. The residue was purified by reverse phase HPLC (column: Waters Xbridge Prep OBD C18 150*40 mm*10 um; mobile phase: [water (NH₄HCO₃)-ACN]; B %: 35%-65%, 8 min) affording 2-((S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-1-((E)-4-(dimethylamino)but-2-enoyl)piperazin-2-yl)acetonitrile (8.2 mg, 5.28%) as an orange solid: ¹H NMR (400 MHz, Chloroform -d) δ 9.09 (s, 1H), 7.52-7.45 (m, 1H), 7.40-7.36 (m, 1H), 7.33-7.28 (m, 1H), 7.05-6.95 (m, 1H), 6.53-6.40 (m, 1H), 5.38-5.18 (m, 1H), 5.12-4.91 (m, 1H), 4.59-4.41 (m, 2H), 4.36-4.30 (m, 1H), 4.26-4.20 (m, 1H), 3.96 (br s, 2H), 3.91-3.69 (m, 2H), 3.29-3.28 (m, 1H), 3.31-3.23 (m, 1H), 3.20-3.10 (m, 3H), 3.03-2.93 (m, 2H), 2.84-2.72 (m, 1H), 2.29 (s, 6H), 2.28-2.25 (m, 1H), 2.23-2.11 (m, 2H), 2.09-2.01 (m, 1H), 2.00-1.84 (m, 3H), 0.80-0.65 (m, 2H), 0.26-0.05 (m, 2H).

LCMS (5% to 95% acetonitrile in water+0.03% ammonium bicarbonate over 6 min); retention time 3.091 min, ESI+found [M+H]=691.3.

### BIOLOGICAL EXAMPLES

### Example 116: Inhibition of KRAS^{G12C} and cRAF Binding

The AlphaScreen technology was used to determine IC_{50S} for compound inhibition of KRAS G12C (present as the Cys-light (C51S, C80L and C118S), truncated version comprising amino acids 1-169) and cRAF interaction. Compounds were diluted in 100% DMSO and each compound concentration was spotted at 200 nl/well onto low volume, white 384 well plates. The KRAS G12C contained a biotin-AviTag and the cRaf, as Ras-binding domain (amino acids 50-131, RBD), was GST-tagged. KRAS G12C was preloaded with the GTP analogue Guanosine 5′-[β,γ-imido]triphosphate (GMPPNP). The KRAS G12C was diluted in 25 mM Hepes, pH 7.4, 150 mM NaCl, 5 mM MgCl₂, 0.01% TritonX-100 and 10 μM GMPPNP and added at 10 ul/well to compound-spotted plates resulting in a DMSO concentration of 2%. Plates were incubated for 2 hours. A mixture of RBD and the AlphaScreen streptavidin donor and glutathione acceptor beads diluted in 25 mM Hepes, pH 7.4, 150 mM NaCl, 5 mM MgCl₂, 0.01% TritonX-100 and 2% DMSO was then added at 10 ul/well and incubated for 60-90 minutes before the samples were read for emission at 570 nm after excitation of the donor beads at 680 nm. All incubations were performed at room temperature. The final top compound concentration was 50 μM with 1:3 titrations for 10-point dose response curves. Final assay conditions were 0.5 nM KRAS G12C, 0.75 nM RBD and 5 μg/ml each of AlphaScreen donor and acceptor beads. IC_{50S} were determined using nonlinear regression fit of [inhibitor] vs. response (4 parameters).

A counter assay was also set up to rule out inhibitors of the AlphaScreen technology itself. Compound plates were incubated for 2 hours as above with buffer only. The AlphaScreen beads were added as above except biotin-AviTag-GST was substituted for the RBD. Samples were read and analyzed as above.

Results for compounds are shown in Table 1, Column 5.

### Example 117: Inhibition of KRAS^{G12C} and PI3Ka Binding

The AlphaScreen technology was used to determine IC_{50S} for compound inhibition of KRAS G12C (present as the Cys-light (C51S, C80L and C118S), truncated version comprising amino acids 1-169) and PI3Ka interaction. Compounds were diluted in 100% DMSO and each compound concentration was spotted at 200 nl/well onto low volume, white 384 well plates. The KRAS G12C contained a biotin-AviTag and the PI3Ka, as Ras-binding domain (amino acids 157-300, RBD), was His-tagged. KRAS G12C was preloaded with the GTP analogue Guanosine 5′-[β,γ-imido]triphosphate (GMPPNP). The KRAS G12C was diluted in 25 mM Hepes, pH 7.4, 150 mM NaCl, 5 mM MgCl₂, 0.01% TritonX-100 and 10 μM GMPPNP and added at 10 ul/well to compound-spotted plates resulting in a DMSO concentration of 2%. Plates were incubated for 2 hours. A mixture of RBD and the AlphaScreen streptavidin donor and nickel chelate acceptor beads diluted in 25 mM Hepes, pH 7.4, 150 mM NaCl, 5 mM MgCl₂, 0.01% TritonX-100 and 2% DMSO was then added at 10 ul/well and incubated for 60-90 minutes before the samples were read for emission at 570 nm after excitation of the donor beads at 680 nm. All incubations were performed at room temperature. The final top compound concentration was 50 μM with 1:3 titrations for 10-point dose response curves. Final assay conditions were 1.5 nM KRAS G12C, 100 nM RBD, 1.25 ug/ml of AlphaScreen donor beads and 10 μg/ml AlphaLISA acceptor beads. IC₅₀ₛ were determined using nonlinear regression fit of [inhibitor] vs. response (4 parameters).

A counter assay was also set up to rule out inhibitors of the AlphaScreen technology itself. Compound plates were incubated for 19-20 hours as above with buffer only. The AlphaScreen beads were added as above except an unrelated biotinylated His-tagged peptide was substituted for the RBD. Samples were read and analyzed as above.

Results for exemplary compounds are shown in Table 1, column 6.

### Example 118: Determination of logD

Preparation of phosphate buffer saturated with 1-octanol: 10 mL of 100 mM phosphate buffer (pH 7.4) was added to 100 mL of 1-octanol. The mixture was shaken vigorously and left to stand at room temperature overnight.

Preparation of 1-octanol saturated with phosphate buffer: 10 mL of 1-octanol was added to 100 mL of 100 mM phosphate buffer (pH 7.4). The mixture was shaken vigorously and left to stand at room temperature overnight.

Procedure for measuring logD: 2 μL of a 10 mM DMSO stock solution of the test compound was aliquoted into 2 separate sample tubes. 149 μL of phosphate buffer saturated with 1-octanol was added into 1^{st} sample tube. 149 μL of 1-octanol saturated with phosphate buffer was added into the 2^{nd} sample tube. The sample tubes were mixed vigorously and shaken for 1 hour at a speed of 800 rpm at room temperature. The sample tubes were subjected to centrifugation at 4000 rpm for 5 minutes at room temperature. Samples of the supernatant were taken from both tubes, diluted with an appropriate LCMS buffer, and analyzed on an LCMS instrument. LogD was calculated according to the following equation.

The data for exemplary compounds is shown in Table 1, column 7.

$$Log {D}_{octanol / buffer} = log ( \frac{Mean Octanol Layer Peak Area Ratio * Octanol Layer Dilution Factor}{Mean Buffer Layer Peak Area Ratio * Buffer Layer Dilution Factor} )$$

### Example 119: Determination of kinetic solubility (pH 7.4)

10 μL of a 10 mM DMSO stock solution of the test compound was aliquoted into the lower chamber of a Whatman Mini-UniPrep vial. 490 μL of a 50 mM phosphate buffer (pH 7.4) was added to the sample solution. The mixture was vortexed for at least 2 minutes. The vial was shaken on a Barnstead shaker at 800 rpm at room temperature for 2 hours. The vial was centrifuged at 4000 rpm for 20 minutes. The Mini-UniPrep vial was compressed to prepare a filtrate, which was injected into an HPLC-UV and LC-MS/MS system. Kinetic solubility was calculated by referencing sample peak areas to a standard calibration curve.

The data for exemplary compounds is shown in Table 1, column 8.

### Example 120: MCF10A (G12C or G12C-A59G)-KRAS cell viability assay

MCF10A (ATCC, cat. CRL-10317) cells are maintained in MEBM (Lonza, cat. CC-3151) with 1% horse serum (Sigma, cat. H1270), MEGM mammary epithelial cell growth medium SingleQuotsKit (Lonza, cat. CC-4146) and 25 ng/ml Cholera toxin (Sigma, cat. C8052). These cells are transduced with either KRAS G12C or G12C/A59G followed by puromycin selection to generate stably expressing cells. For the cell viability assay, 1000 cells of either MCF10A KRAS G12C or MCF10A G12C/A59G are plated in 384-well spheroid microplate (Corning, cat. 3830). The following day, cells are treated with compounds (10 uM top concentration, 3-fold dilution, and 11 doses). 10 uM Tremetinib (MCE, cat. HY-10999/CS-0060) is used as control. The Tecan: HP D300E is used to dispense the compounds. After five days of incubation, celltiter-glo luminescent assay kit (Promega, cat. G7573) is used according to manufacturer's protocol to measure cellular viability using a BioTek plate reader. The data is then imported to and processed in Dotmatics where EC50s were calculated using the Lavenberg-Marquardt 4 parameters fitting procedure, with difference gradients.

### Example 121: Treatment of Human Patients

A human patient suffering from a cancer, (e.g., a KRAS mediated cancer, as disclosed herein) can be administered a therapeutically effective dose of a compound disclosed herein (e.g., a compound of Table 1). The treatment can slow down or halt the growth of a tumor, reduce a tumor volume or mass, or eradicate the tumor in the patient.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

TABLE 1
Exemplary compounds and experimental data

| | | | | Raf- | | | |
|---|---|---|---|---|---|---|---|
| | | | | 1 | PI3K | | Ksol, |
| | | | | RBD | RBD | Log | pH |
| | | Com- | | K- | K- | D, | 7.4 |
| | Stereo- | pound | Exam- | Ras | Ras | pH | (ug/ |
| | chemistry | Nr. | ple | IC50 | IC50 | 7.4 | mL) |
| 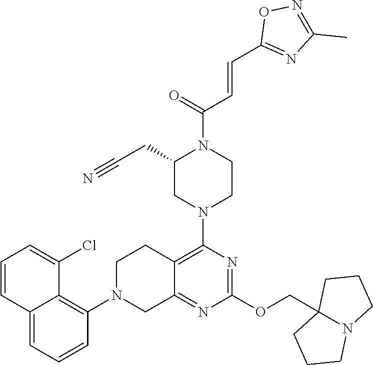 [SMILES: Cc1noc(/C=C/C(=O)N2CCN(c3nc(OCC45CCCN4CCC5)nc4c3CCN(c3cccc5cccc(Cl)c35)C4)C[C@@H]2CC#N)n1] | | 1 | 1 | +++ | ++++ | 2.90 | 111.20 |
| 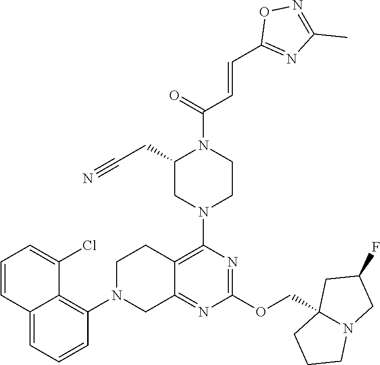 [SMILES: Cc1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1] | | 2 | 2 | +++ | ++++ | 4.42 | |
| 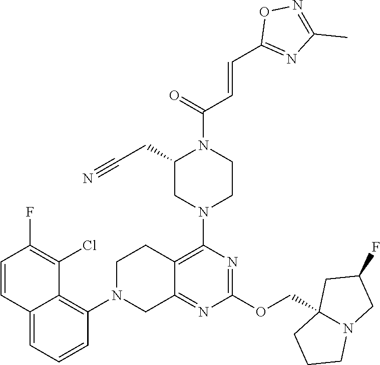 [SMILES: Cc1noc(/C=C/C(=O)N2CCN(c3nc(OC[C@@]45CCCN4C[C@H](F)C5)nc4c3CCN(c3cccc5ccc(F)c(Cl)c35)C4)C[C@@H]2CC#N)n1] | | 3 | 3 | +++ | ++++ | 4.44 | |
| 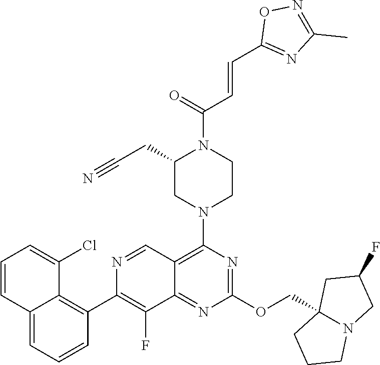 [SMILES: Cc1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1] | | 4 | 4 | +++ | +++ | 3.54 | 3.98 |
| 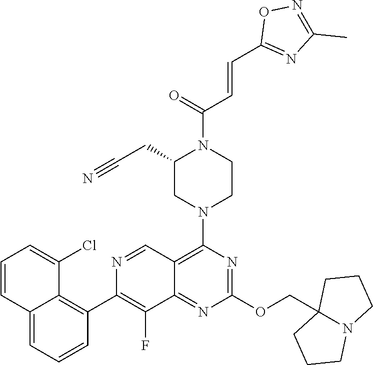 [SMILES: Cc1noc(/C=C/C(=O)N2CCN(c3nc(OCC45CCCN4CCC5)nc4c(F)c(-c5cccc6cccc(Cl)c56)ncc34)C[C@@H]2CC#N)n1] | | 5 | 5 | +++ | | 2.15 | 161.06 |
| 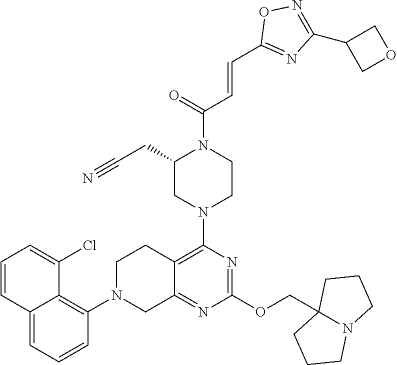 [SMILES: N#CC[C@H]1CN(c2nc(OCC34CCCN3CCC4)nc3c2CCN(c2cccc4cccc(Cl)c24)C3)CCN1C(=O)/C=C/c1nc(C2COC2)no1] | | 6 | 6 | +++ | | 2.72 | 86.37 |
| 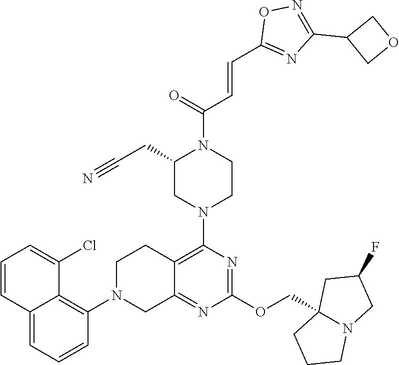 [SMILES: N#CC[C@H]1CN(c2nc(OC[C@@]34CCCN3C[C@H](F)C4)nc3c2CCN(c2cccc4cccc(Cl)c24)C3)CCN1C(=O)/C=C/c1nc(C2COC2)no1] | | 7 | 7 | ++++ | | 2.69 | 0.01 |
| 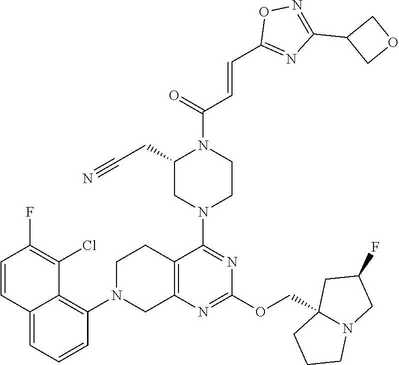 [SMILES: N#CC[C@H]1CN(c2nc(OC[C@@]34CCCN3C[C@H](F)C4)nc3c2CCN(c2cccc4ccc(F)c(Cl)c24)C3)CCN1C(=O)/C=C/c1nc(C2COC2)no1] | | 8 | 8 | ++++ | ++++ | 3.78 | 0.01 |
| 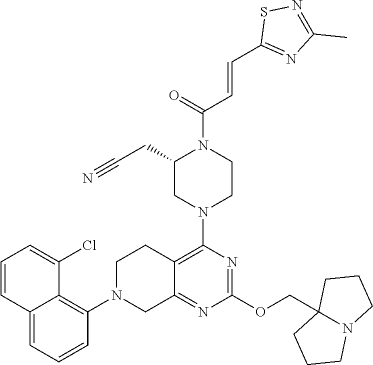 [SMILES: Cc1nsc(/C=C/C(=O)N2CCN(c3nc(OCC45CCCN4CCC5)nc4c3CCN(c3cccc5cccc(Cl)c35)C4)C[C@@H]2CC#N)n1] | | 9 | 9 | ++++ | ++++ | 3.55 | 67.03 |
| 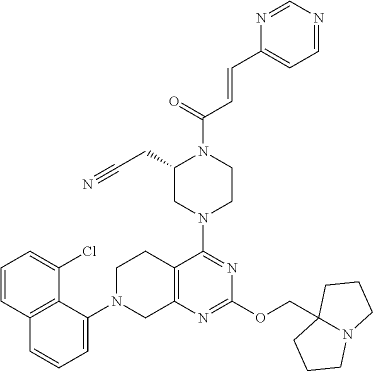 [SMILES: N#CC[C@H]1CN(c2nc(OCC34CCCN3CCC4)nc3c2CCN(c2cccc4cccc(Cl)c24)C3)CCN1C(=O)/C=C/c1ccncn1] | | 10 | 10 | ++++ | ++++ | 2.93 | 99.03 |
| 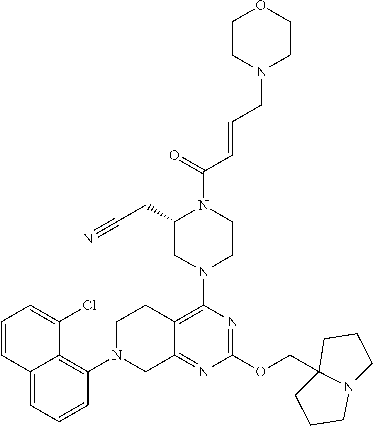 [SMILES: N#CC[C@H]1CN(c2nc(OCC34CCCN3CCC4)nc3c2CCN(c2cccc4cccc(Cl)c24)C3)CCN1C(=O)/C=C/CN1CCOCC1] | | 11 | 11 | ++++ | +++ | 2.36 | 89.48 |
| 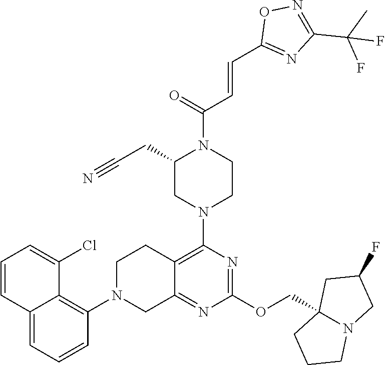 [SMILES: CC(F)(F)c1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1] | | 12 | 12 | ++++ | ++++ | 5.10 | 0.01 |
| 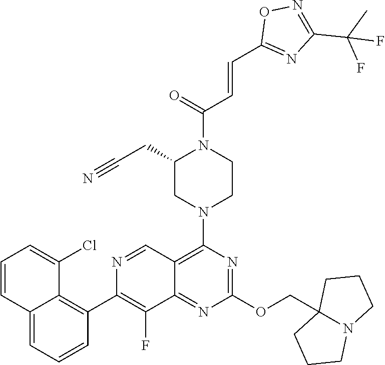 [SMILES: CC(F)(F)c1noc(/C=C/C(=O)N2CCN(c3nc(OCC45CCCN4CCC5)nc4c(F)c(-c5cccc6cccc(Cl)c56)ncc34)C[C@@H]2CC#N)n1] | | 13 | 13 | +++ | +++ | | |
| 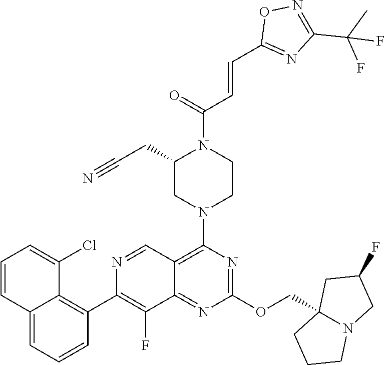 [SMILES: CC(F)(F)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1] | | 14 | 14 | ++++ | ++++ | 4.49 | 0.04 |
| 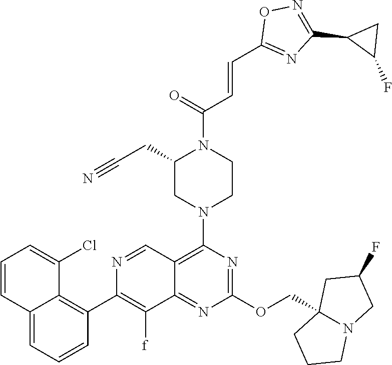 | (abs) piperazine, (and) fluorocyclo- propyl | 15 | 15 | ++ | | 4.24 | 0.01 |
| 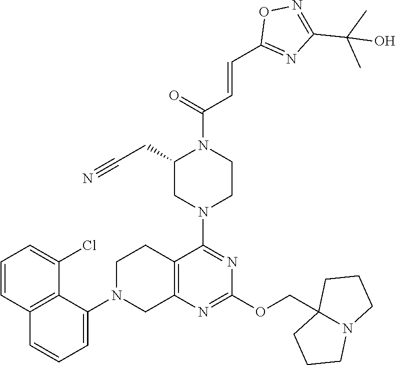 [SMILES: CC(C)(O)c1noc(/C=C/C(=O)N2CCN(c3nc(OCC45CCCN4CCC5)nc4c3CCN(c3cccc5cccc(Cl)c35)C4)C[C@@H]2CC#N)n1] | | 16 | 16 | +++ | | 3.04 | 73.07 |
| 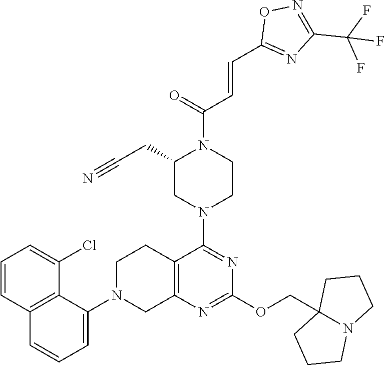 [SMILES: N#CC[C@H]1CN(c2nc(OCC34CCCN3CCC4)nc3c2CCN(c2cccc4cccc(Cl)c24)C3)CCN1C(=O)/C=C/c1nc(C(F)(F)F)no1] | | 17 | 17 | ++++ | ++++ | 4.32 | 0.01 |
| 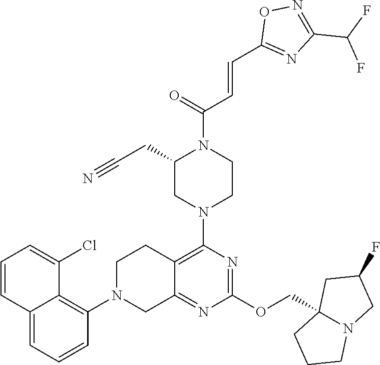 [SMILES: N#CC[C@H]1CN(c2nc(OC[C@@]34CCCN3C[C@H](F)C4)nc3c2CCN(c2cccc4cccc(Cl)c24)C3)CCN1C(=O)/C=C/c1nc(C(F)F)no1] | | 18 | | ++++ | ++++ | 4.78 | 0.01 |
| 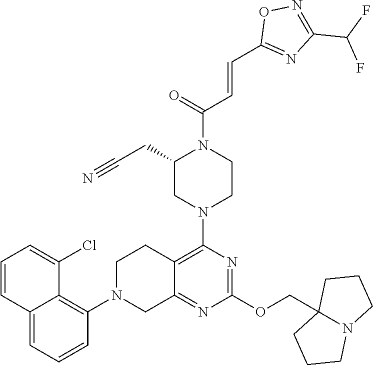 [SMILES: N#CC[C@H]1CN(c2nc(OCC34CCCN3CCC4)nc3c2CCN(c2cccc4cccc(Cl)c24)C3)CCN1C(=O)/C=C/c1nc(C(F)F)no1] | | 19 | | ++++ | ++++ | 3.33 | 24.43 |
| 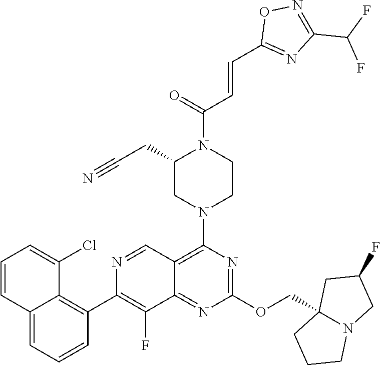 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/c1nc(C(F)F)no1] | | 20 | | ++++ | | 2.61 | 0.28 |
| 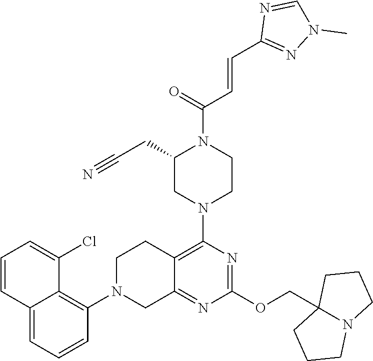 [SMILES: Cn1cnc(/C=C/C(=O)N2CCN(c3nc(OCC45CCCN4CCC5)nc4c3CCN(c3cccc5cccc(Cl)c35)C4)C[C@@H]2CC#N)n1] | | 21 | | + | | 2.48 | 89.81 |
| 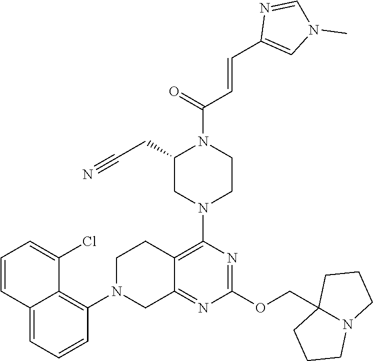 [SMILES: Cn1cnc(/C=C/C(=O)N2CCN(c3nc(OCC45CCCN4CCC5)nc4c3CCN(c3cccc5cccc(Cl)c35)C4)C[C@@H]2CC#N)c1] | | 22 | | + | | 2.92 | 77.71 |
| 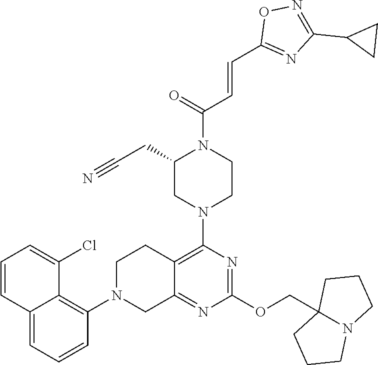 [SMILES: N#CC[C@H]1CN(c2nc(OCC34CCCN3CCC4)nc3c2CCN(c2cccc4cccc(Cl)c24)C3)CCN1C(=O)/C=C/c1nc(C2CC2)no1] | | 23 | | +++ | +++ | 3.38 | 12.48 |
| 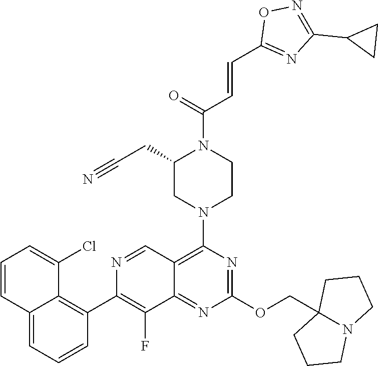 [SMILES: N#CC[C@H]1CN(c2nc(OCC34CCCN3CCC4)nc3c(F)c(-c4cccc5cccc(Cl)c45)ncc23)CCN1C(=O)/C=C/c1nc(C2CC2)no1] | | 24 | | ++ | | 2.71 | 38.47 |
| 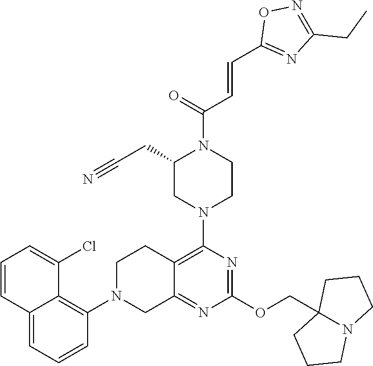 [SMILES: CCc1noc(/C=C/C(=O)N2CCN(c3nc(OCC45CCCN4CCC5)nc4c3CCN(c3cccc5cccc(Cl)c35)C4)C[C@@H]2CC#N)n1] | | 25 | | + | + | 4.90 | 0.01 |
| 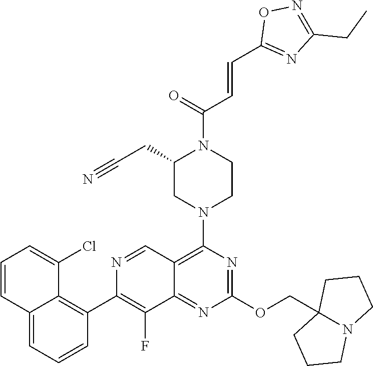 [SMILES: CCc1noc(/C=C/C(=O)N2CCN(c3nc(OCC45CCCN4CCC5)nc4c(F)c(-c5cccc6cccc(Cl)c56)ncc34)C[C@@H]2CC#N)n1] | | 26 | | ++ | ++ | 2.66 | 80.19 |
| 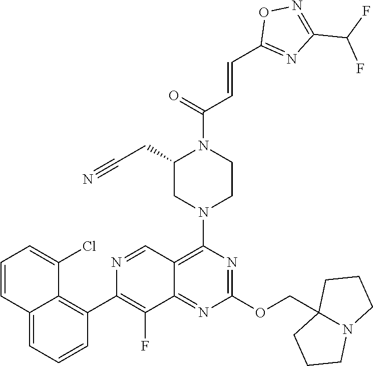 [SMILES: N#CC[C@H]1CN(c2nc(OCC34CCCN3CCC4)nc3c(F)c(-c4cccc5cccc(Cl)c45)ncc23)CCN1C(=O)/C=C/c1nc(C(F)F)no1] | | 27 | | ++++ | | 2.52 | 102.69 |
| 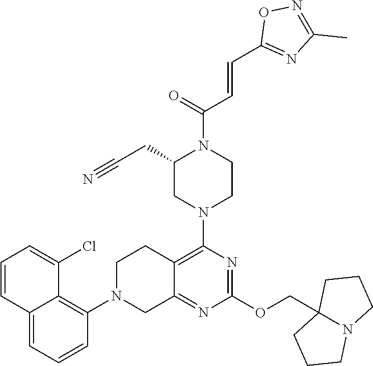 [SMILES: Cc1noc(/C=C/C(=O)N2CCN(c3nc(OCC45CCCN4CCC5)nc4c3CCN(c3cccc5cccc(Cl)c35)C4)C[C@@H]2CC#N)n1] | | 28 | | ++ | | 3.22 | 43.66 |
| 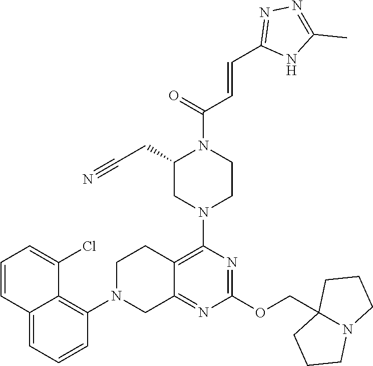 [SMILES: Cc1nnc(/C=C/C(=O)N2CCN(c3nc(OCC45CCCN4CCC5)nc4c3CCN(c3cccc5cccc(Cl)c35)C4)C[C@@H]2CC#N)[nH]1] | | 29 | | + | | 2.84 | 74.65 |
| 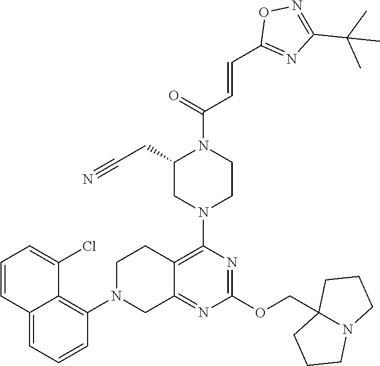 [SMILES: CC(C)(C)c1noc(/C=C/C(=O)N2CCN(c3nc(OCC45CCCN4CCC5)nc4c3CCN(c3cccc5cccc(Cl)c35)C4)C[C@@H]2CC#N)n1] | | 30 | | +++ | +++ | 3.09 | 2.33 |
| 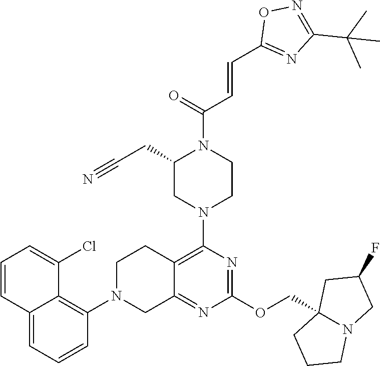 [SMILES: CC(C)(C)c1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1] | | 31 | | +++ | +++ | 4.35 | 0.01 |
| 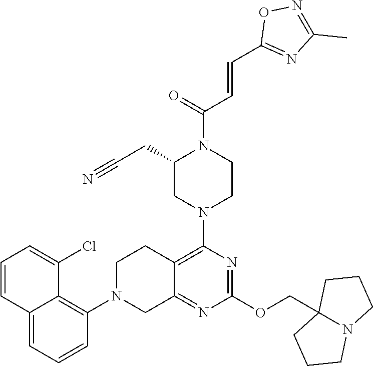 [SMILES: Cc1noc(/C=C/C(=O)N2CCN(c3nc(OCC45CCCN4CCC5)nc4c3CCN(c3cccc5cccc(Cl)c35)C4)C[C@@H]2CC#N)n1] | | 32 | | +++ | | 3.62 | 79.78 |
| 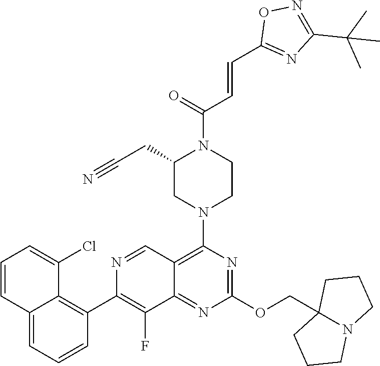 [SMILES: CC(C)(C)c1noc(/C=C/C(=O)N2CCN(c3nc(OCC45CCCN4CCC5)nc4c(F)c(-c5cccc6cccc(Cl)c56)ncc34)C[C@@H]2CC#N)n1] | | 33 | | ++ | | 3.73 | 50.03 |
| 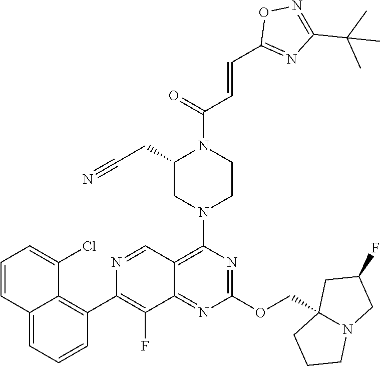 [SMILES: CC(C)(C)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1] | | 34 | | ++ | | 5.11 | 0.01 |
| 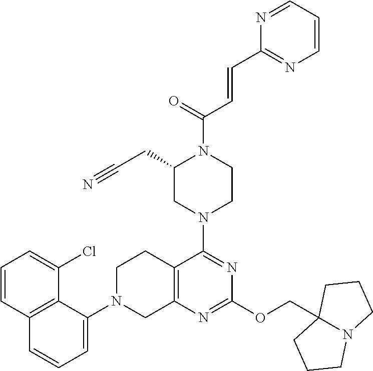 [SMILES: N#CC[C@H]1CN(c2nc(OCC34CCCN3CCC4)nc3c2CCN(c2cccc4cccc(Cl)c24)C3)CCN1C(=O)/C=C/c1ncccn1] | | 35 | | ++ | | 2.77 | 111.43 |
| 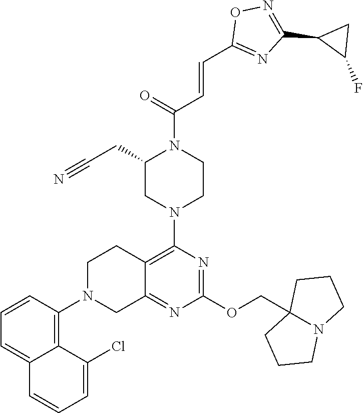 [SMILES: N#CC[C@H]1CN(c2nc(OCC34CCCN3CCC4)nc3c2CCN(c2cccc4cccc(Cl)c24)C3)CCN1C(=O)/C=C/c1nc([C@H]2C[C@@H]2F)no1] | (abs) piperazine, (and) fluorocyclo- propyl | 36 | | +++ | | 3.98 | 2.62 |
| 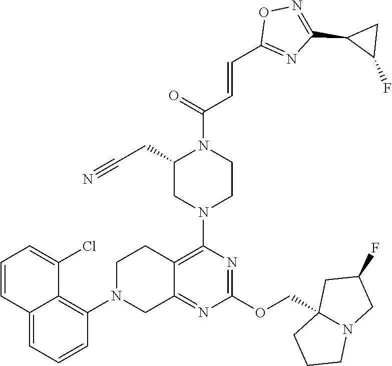 [SMILES: N#CC[C@H]1CN(c2nc(OC[C@@]34CCCN3C[C@H](F)C4)nc3c2CCN(c2cccc4cccc(Cl)c24)C3)CCN1C(=O)/C=C/c1nc([C@H]2C[C@@H]2F)no1] | (abs) piperazine, (and) fluorocyclo- propyl | 37 | | +++ | | 4.85 | 0.01 |
| 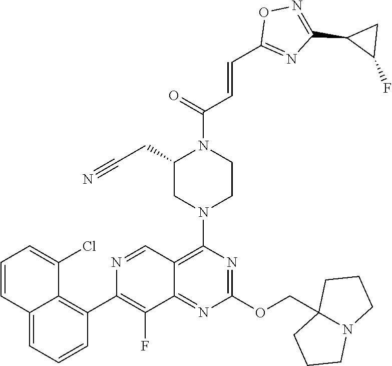 [SMILES: N#CC[C@H]1CN(c2nc(OCC34CCCN3CCC4)nc3c(F)c(-c4cccc5cccc(Cl)c45)ncc23)CCN1C(=O)/C=C/c1nc([C@H]2C[C@@H]2F)no1] | (abs) piperazine, (and) fluorocyclo- propyl | 38 | | ++ | | 2.91 | 53.91 |
| 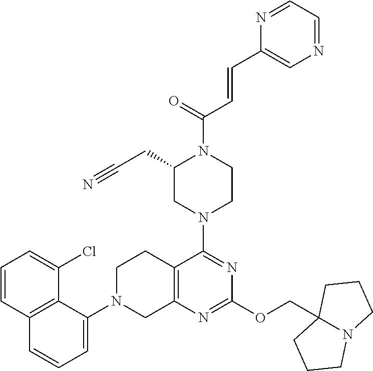 [SMILES: N#CC[C@H]1CN(c2nc(OCC34CCCN3CCC4)nc3c2CCN(c2cccc4cccc(Cl)c24)C3)CCN1C(=O)/C=C/c1cnccn1] | | 39 | | + | | | |
| 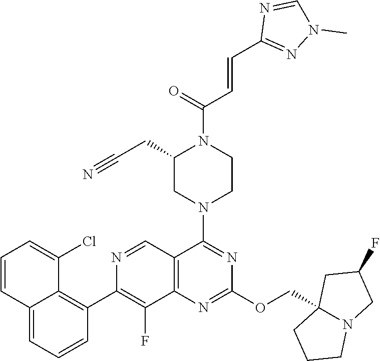 [SMILES: Cn1cnc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1] | | 40 | | + | | | |
| 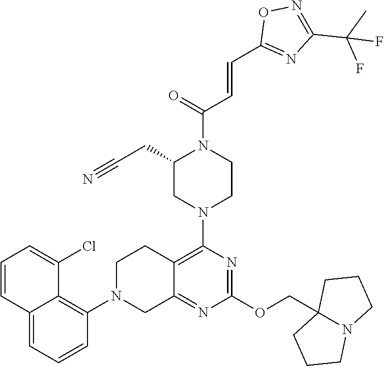 [SMILES: CC(F)(F)c1noc(/C=C/C(=O)N2CCN(c3nc(OCC45CCCN4CCC5)nc4c3CCN(c3cccc5cccc(Cl)c35)C4)C[C@@H]2CC#N)n1] | | 41 | | +++ | | | |
| 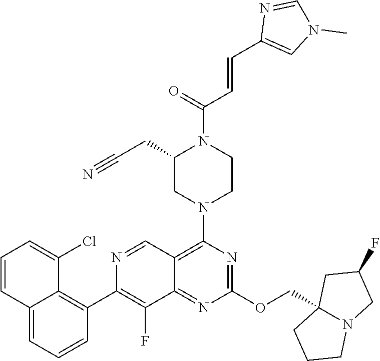 [SMILES: Cn1cnc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)c1] | | 42 | | + | | | |
| 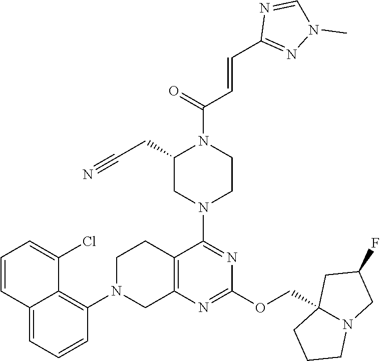 [SMILES: Cn1cnc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1] | | 43 | | + | | | |
| 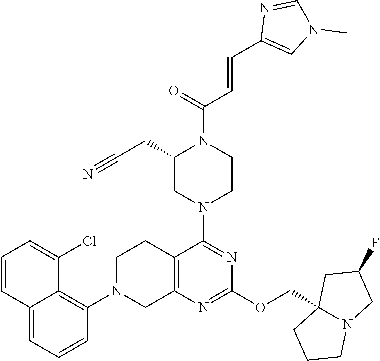 [SMILES: Cn1cnc(/C=C/C(=O)N2CCN(c3nc(OC[C@@]45CCCN4C[C@H](F)C5)nc4c3CCN(c3cccc5cccc(Cl)c35)C4)C[C@@H]2CC#N)c1] | | 44 | | ++ | | | |
| 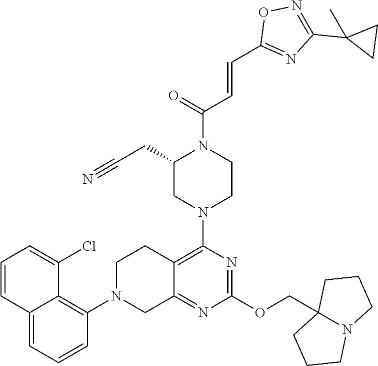 [SMILES: CC1(c2noc(/C=C/C(=O)N3CCN(c4nc(OCC56CCCN5CCC6)nc5c4CCN(c4cccc6cccc(Cl)c46)C5)C[C@@H]3CC#N)n2)CC1] | | 45 | | +++ | | | |
| 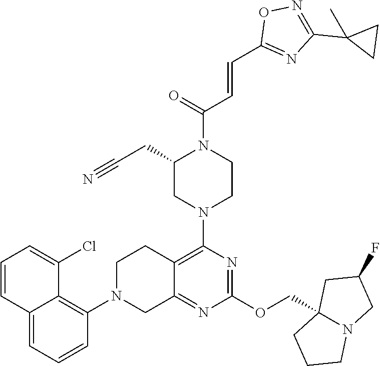 [SMILES: CC1(c2noc(/C=C/C(=O)N3CCN(c4nc(OC[C@@]56CCCN5C[C@H](F)C6)nc5c4CCN(c4cccc6cccc(Cl)c46)C5)C[C@@H]3CC#N)n2)CC1] | | 46 | | +++ | | | |
| 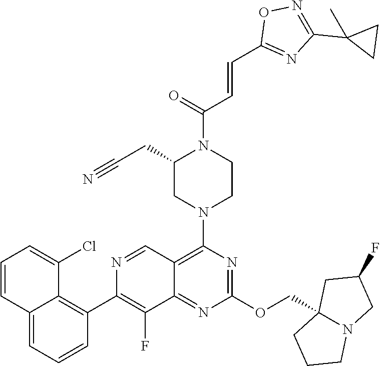 [SMILES: CC1(c2noc(/C=C/C(=O)N3CCN(c4nc(OC[C@@]56CCCN5C[C@H](F)C6)nc5c(F)c(-c6cccc7cccc(Cl)c67)ncc45)C[C@@H]3CC#N)n2)CC1] | | 47 | | ++ | | 4.59 | 0.01 |
| 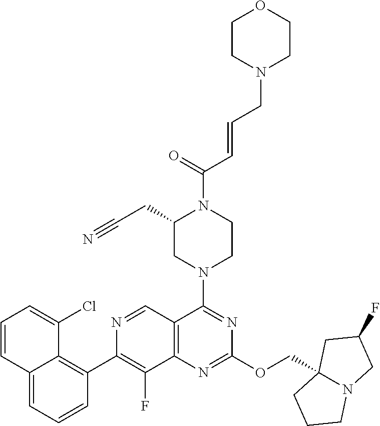 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCOCC1] | | 48 | | +++ | +++ | 2.71 | 109.97 |
| 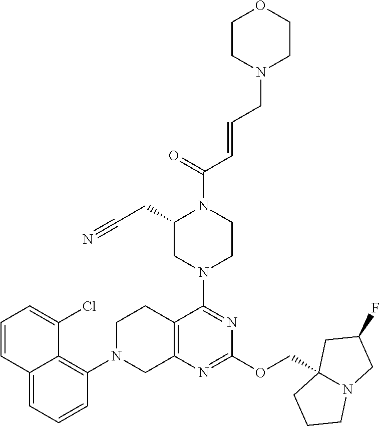 [SMILES: N#CC[C@H]1CN(c2nc(OC[C@@]34CCCN3C[C@H](F)C4)nc3c2CCN(c2cccc4cccc(Cl)c24)C3)CCN1C(=O)/C=C/CN1CCOCC1] | | 49 | | ++++ | ++++ | 3.72 | 45.23 |
| 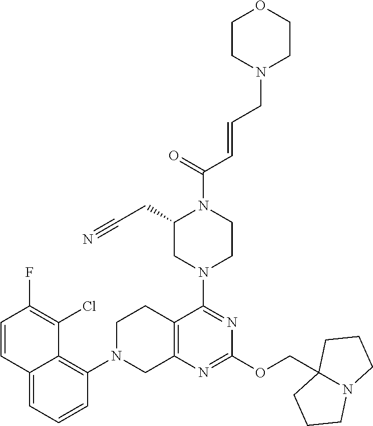 [SMILES: N#CC[C@H]1CN(c2nc(OCC34CCCN3CCC4)nc3c2CCN(c2cccc4ccc(F)c(Cl)c24)C3)CCN1C(=O)/C=C/CN1CCOCC1] | | 50 | | ++++ | ++++ | 2.27 | 124.40 |
| 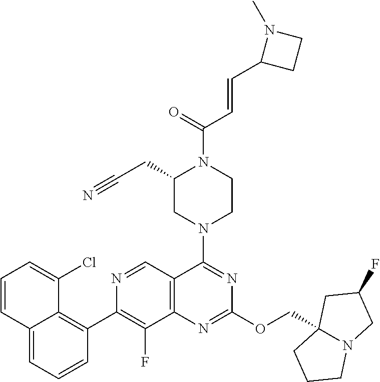 [SMILES: CN1CCC1/C=C/C(=O)N1CCN(c2nc(OC[C@@]34CCCN3C[C@H](F)C4)nc3c(F)c(-c4cccc5cccc(Cl)c45)ncc23)C[C@@H]1CC#N] | | 51 | | +++ | +++ | | |
| 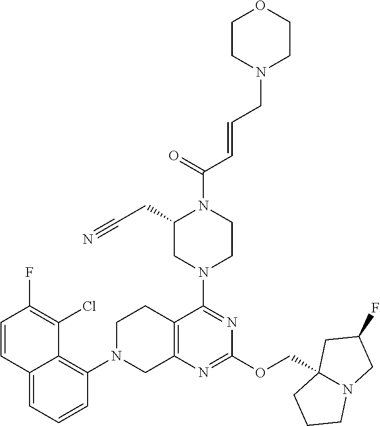 [SMILES: N#CC[C@H]1CN(c2nc(OC[C@@]34CCCN3C[C@H](F)C4)nc3c2CCN(c2cccc4ccc(F)c(Cl)c24)C3)CCN1C(=O)/C=C/CN1CCOCC1] | | 52 | | ++++ | ++++ | 3.80 | 23.79 |
| 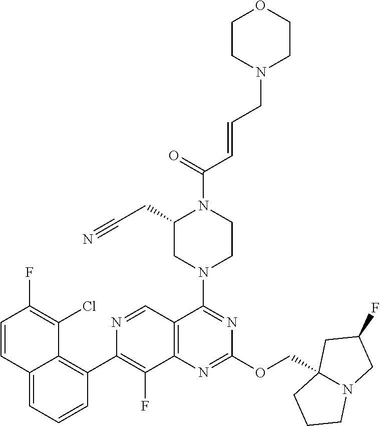 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCOCC1] | | 53 | | ++++ | ++++ | 2.82 | 110.96 |
| 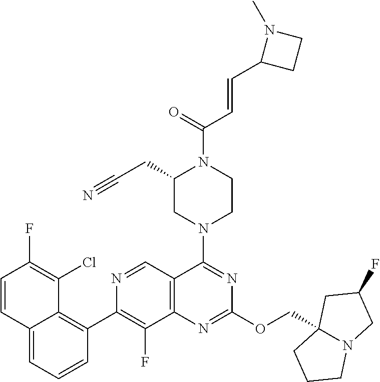 [SMILES: CN1CCC1/C=C/C(=O)N1CCN(c2nc(OC[C@@]34CCCN3C[C@H](F)C4)nc3c(F)c(-c4cccc5ccc(F)c(Cl)c45)ncc23)C[C@@H]1CC#N] | | 54 | | ++++ | ++++ | 2.59 | 135.11 |
| 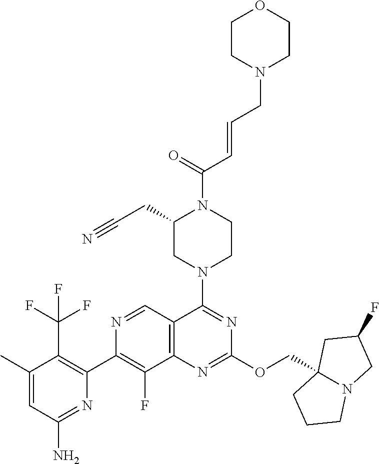 [SMILES: Cc1cc(N)nc(-c2ncc3c(N4CCN(C(=O)/C=C/CN5CCOCC5)[C@@H](CC#N)C4)nc(OC[C@@]45CCCN4C[C@H](F)C5)nc3c2F)c1C(F)(F)F] | | 55 | | +++ | +++ | 1.53 | 128.27 |
| 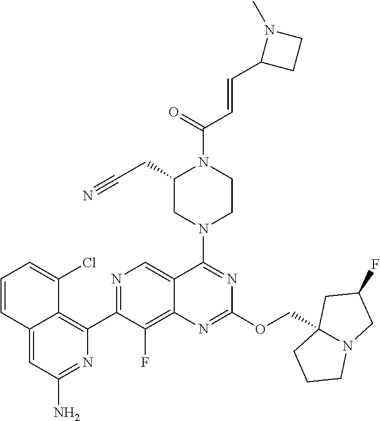 [SMILES: CN1CCC1/C=C/C(=O)N1CCN(c2nc(OC[C@@]34CCCN3C[C@H](F)C4)nc3c(F)c(-c4nc(N)cc5cccc(Cl)c45)ncc23)C[C@@H]1CC#N] | | 56 | * | | | | |
| 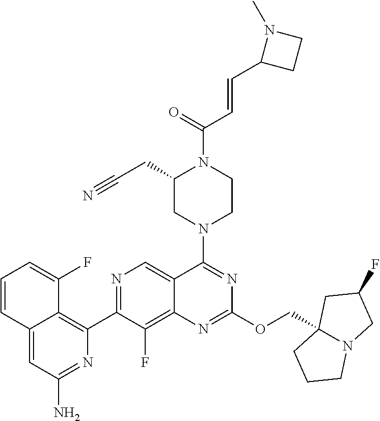 [SMILES: CN1CCC1/C=C/C(=O)N1CCN(c2nc(OC[C@@]34CCCN3C[C@H](F)C4)nc3c(F)c(-c4nc(N)cc5cccc(F)c45)ncc23)C[C@@H]1CC#N] | | 57 | * | | | | |
| 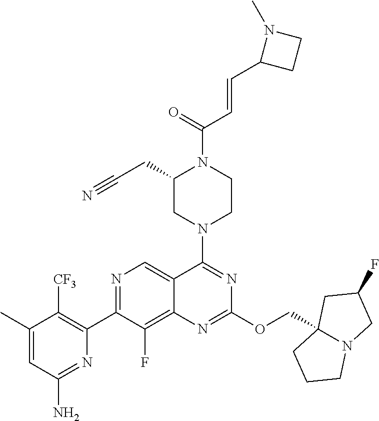 [SMILES: Cc1cc(N)nc(-c2ncc3c(N4CCN(C(=O)/C=C/C5CCN5C)[C@@H](CC#N)C4)nc(OC[C@@]45CCCN4C[C@H](F)C5)nc3c2F)c1C(F)(F)F] | | 58 | * | | | | |
| 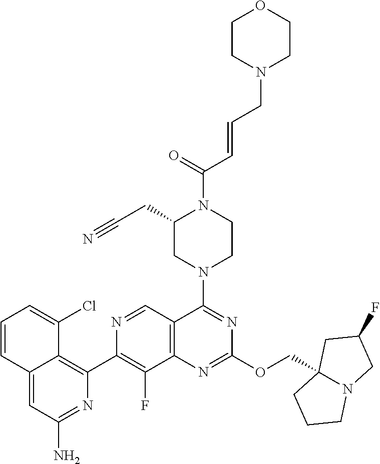 [SMILES: N#CC[C@H]1CN(c2nc(OC[C@@]34CCCN3C[C@H](F)C4)nc3c(F)c(-c4nc(N)cc5cccc(Cl)c45)ncc23)CCN1C(=O)/C=C/CN1CCOCC1] | | 59 | | ++++ | | | |
| 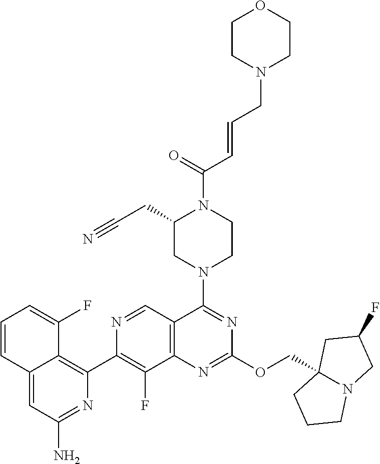 [SMILES: N#CC[C@H]1CN(c2nc(OC[C@@]34CCCN3C[C@H](F)C4)nc3c(F)c(-c4nc(N)cc5cccc(F)c45)ncc23)CCN1C(=O)/C=C/CN1CCOCC1] | | 60 | | ++++ | | 1.08 | 94.76 |
| 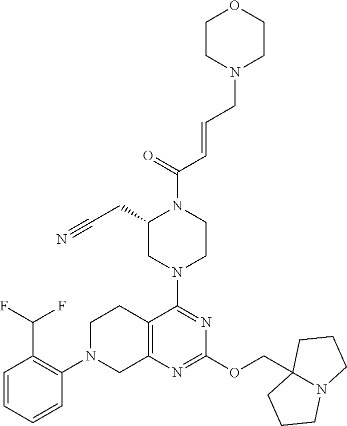 [SMILES: N#CC[C@H]1CN(c2nc(OCC34CCCN3CCC4)nc3c2CCN(c2ccccc2C(F)F)C3)CCN1C(=O)/C=C/CN1CCOCC1] | | 61 | | + | | 1.22 | 88.71 |
| 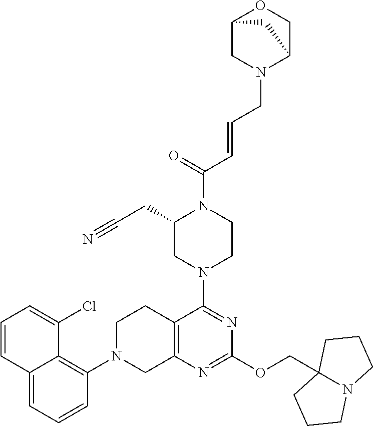 [SMILES: N#CC[C@H]1CN(c2nc(OCC34CCCN3CCC4)nc3c2CCN(c2cccc4cccc(Cl)c24)C3)CCN1C(=O)/C=C/CN1C[C@@H]2C[C@H]1CO2] | | 62 | | ++++ | | 2.06 | 95.06 |
| 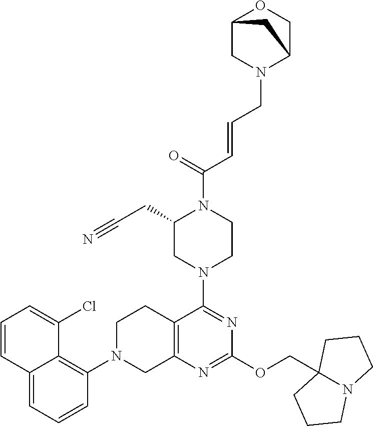 [SMILES: N#CC[C@H]1CN(c2nc(OCC34CCCN3CCC4)nc3c2CCN(c2cccc4cccc(Cl)c24)C3)CCN1C(=O)/C=C/CN1C[C@H]2C[C@@H]1CO2] | | 63 | | ++++ | | | |
| 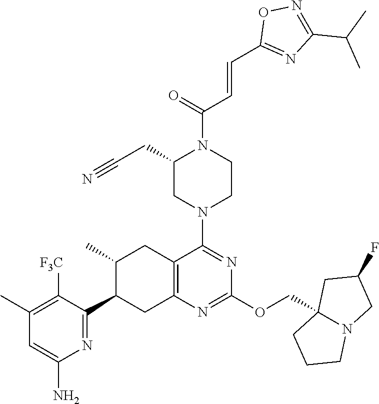 [SMILES: Cc1cc(N)nc([C@@H]2Cc3nc(OC[C@@]45CCCN4C[C@H](F)C5)nc(N4CCN(C(=O)/C=C/c5nc(C(C)C)no5)[C@@H](CC#N)C4)c3C[C@H]2C)c1C(F)(F)F] | (and) cyclohexenyl centers, (abs) piperazine, (abs) pyrrolizine | 66 | 66 | +++ | +++ | 5.02 | 0.08 |
| 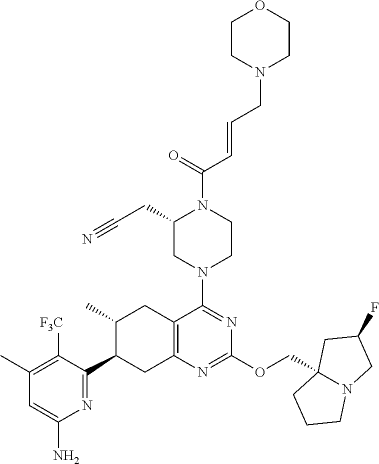 [SMILES: Cc1cc(N)nc([C@@H]2Cc3nc(OC[C@@]45CCCN4C[C@H](F)C5)nc(N4CCN(C(=O)/C=C/CN5CCOCC5)[C@@H](CC#N)C4)c3C[C@H]2C)c1C(F)(F)F] | (and) cyclohexenyl centers, (abs) piperazine, (abs) pyrrolizine | 67 | 67 | +++ | | 2.98 | 78.19 |
| 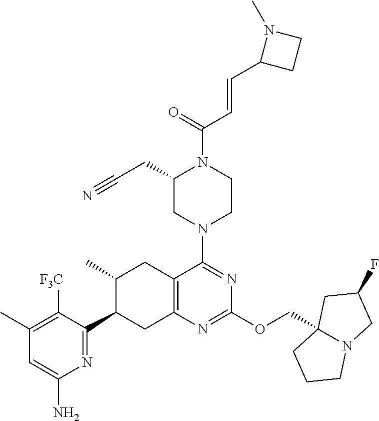 [SMILES: Cc1cc(N)nc([C@@H]2Cc3nc(OC[C@@]45CCCN4C[C@H](F)C5)nc(N4CCN(C(=O)/C=C/C5CCN5C)[C@@H](CC#N)C4)c3C[C@H]2C)c1C(F)(F)F] | (and) cyclohexenyl centers, (abs) piperazine, (abs) pyrrolizine | 68 | 68 | ++++ | ++++ | 3.09 | 101.62 |
| 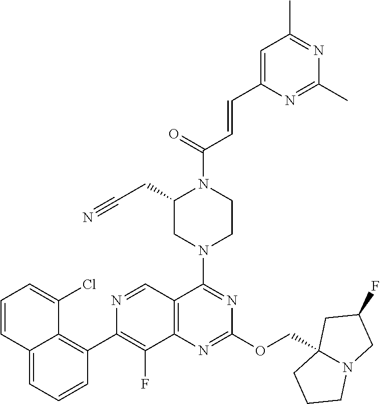 [SMILES: Cc1cc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)nc(C)n1] | | 69 | 69 | ++++ | ++++ | 3.86 | 5.74 |
| 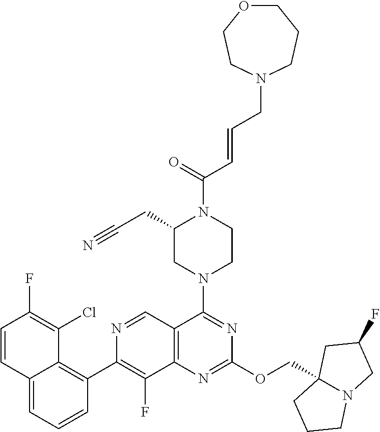 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCCOCC1] | | 70 | 70 | ++++ | ++++ | 3.25 | 123.89 |
| 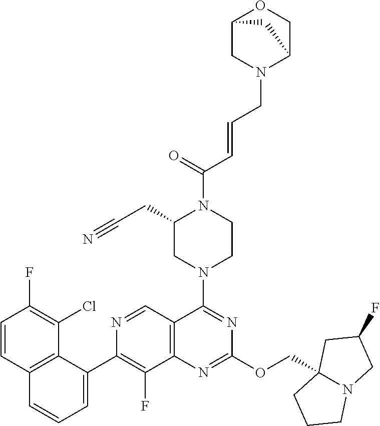 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@@H]2C[C@H]1CO2] | | 71 | 71 | ++++ | ++++ | 2.90 | 88.90 |
| 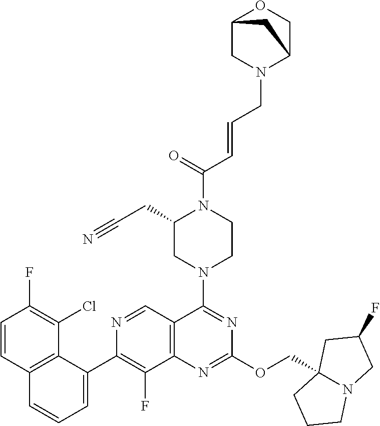 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@H]2C[C@@H]1CO2] | | 72 | 72 | ++++ | ++++ | 3.12 | 143.17 |
| 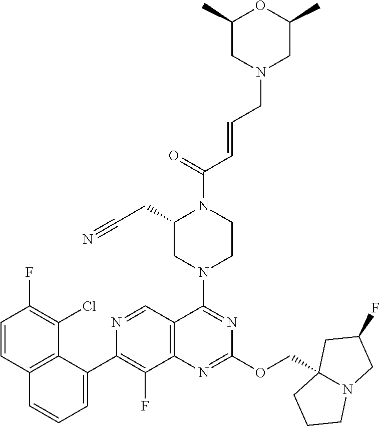 [SMILES: C[C@@H]1CN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C[C@H](C)O1] | | 73 | 73 | ++++ | ++++ | 3.84 | 105.26 |
| 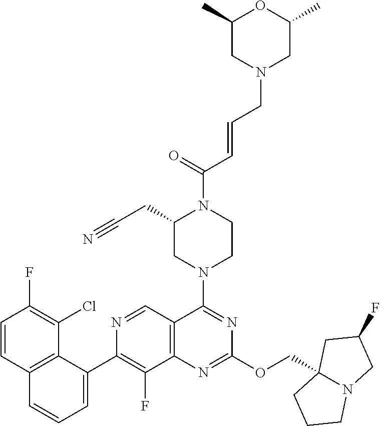 [SMILES: C[C@@H]1CN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C[C@@H](C)O1] | | 74 | 74 | ++++ | ++++ | 4.18 | 76.18 |
| 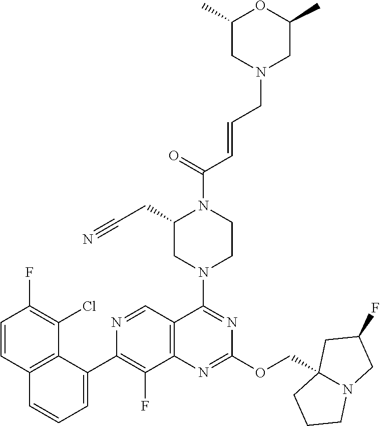 [SMILES: C[C@H]1CN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C[C@H](C)O1] | | 75 | 75 | ++++ | ++++ | 4.06 | 76.88 |
| 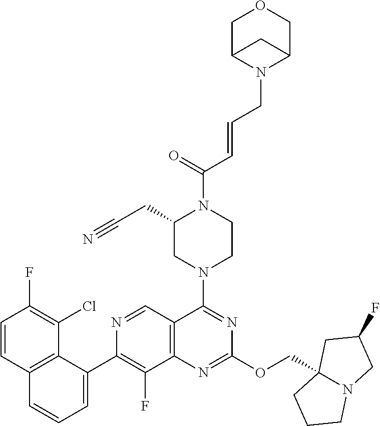 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C2COCC1C2] | | 76 | 76 | ++++ | ++++ | 3.19 | 110.71 |
| 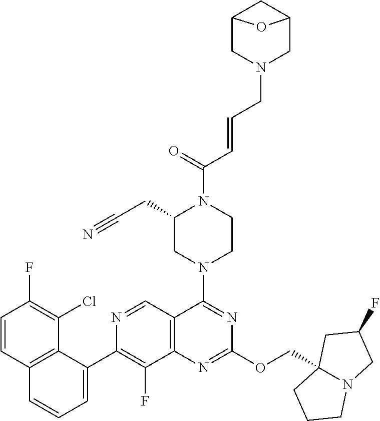 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2CC(C1)O2] | | 77 | 77 | ++++ | ++++ | 2.92 | 116.42 |
| 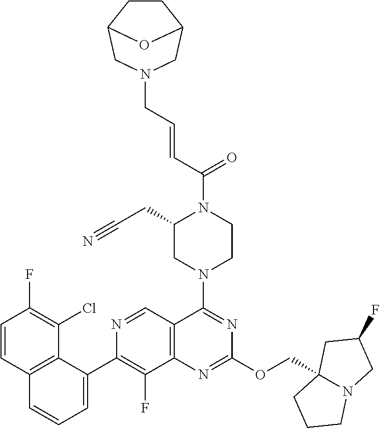 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2CCC(C1)O2] | | 78 | 78 | ++++ | ++++ | 3.64 | 85.00 |
| 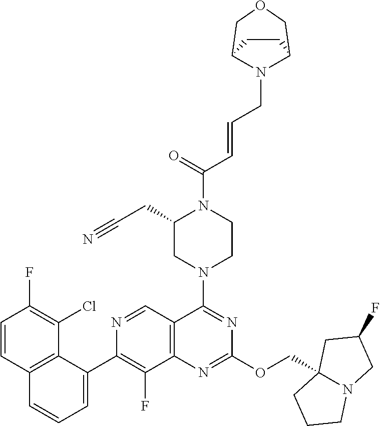 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1[C@@H]2CC[C@H]1COC2] | | 79 | 79 | ++++ | ++++ | 3.53 | 154.82 |
| 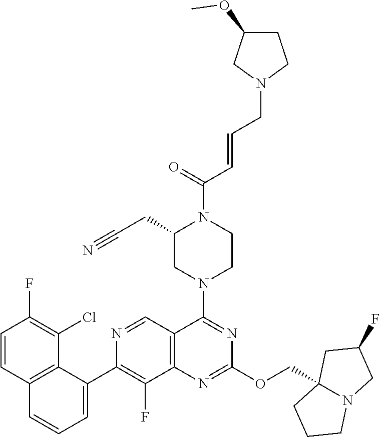 [SMILES: CO[C@H]1CCN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C1] | | 80 | 80 | ++++ | ++++ | 3.20 | 146.44 |
| 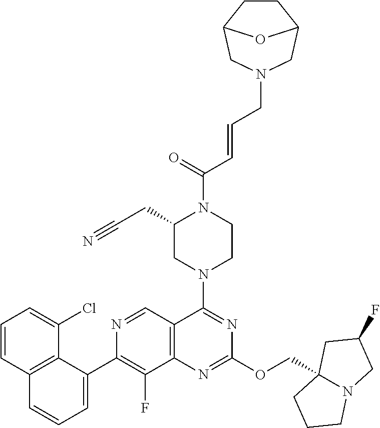 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CCC(C1)O2] | | 81 | 81 | ++++ | ++++ | 3.70 | 74.42 |
| 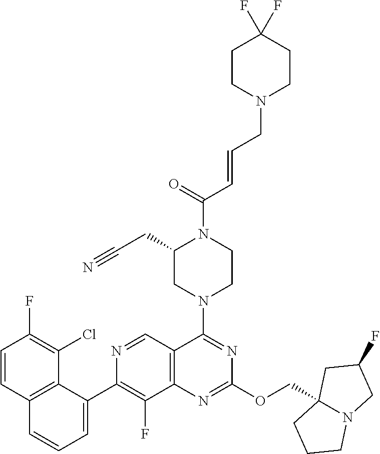 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCC(F)(F)CC1] | | 82 | 82 | ++++ | ++++ | 4.20 | 3.46 |
| 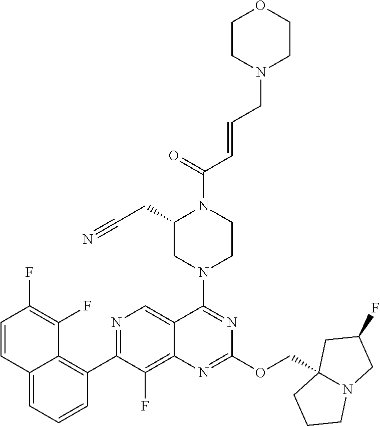 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCOCC1] | | 83 | 83 | ++++ | ++++ | 2.98 | 93.39 |
| 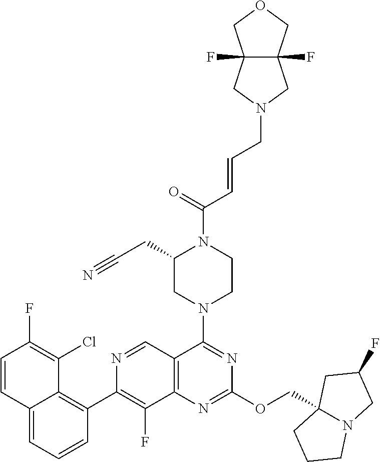 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@]2(F)COC[C@]2(F)C1] | | 84 | 84 | ++++ | ++++ | 2.97 | 81.53 |
| 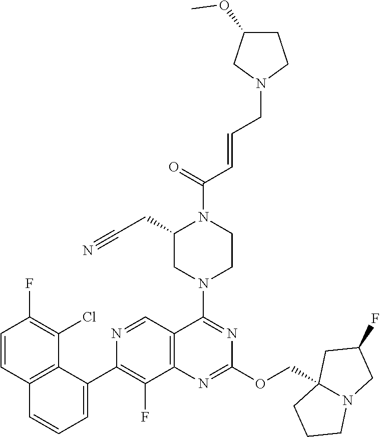 [SMILES: CO[C@@H]1CCN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C1] | | 85 | 85 | ++++ | ++++ | 2.97 | 85.46 |
| 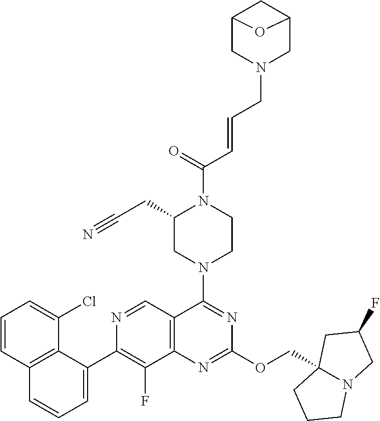 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CC(C1)O2] | | 86 | 86 | ++++ | ++++ | 2.90 | 96.61 |
| 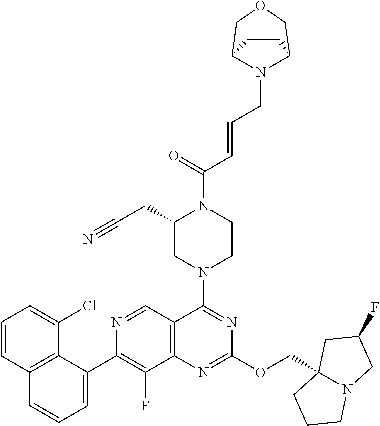 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1[C@@H]2CC[C@H]1COC2] | | 87 | 87 | ++++ | ++++ | 3.27 | 69.81 |
| 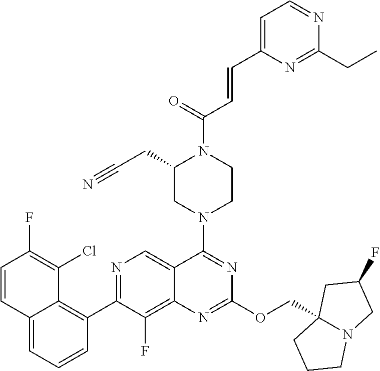 [SMILES: CCc1nccc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1] | | 88 | 88 | ++++ | ++++ | 4.55 | 45.85 |
| 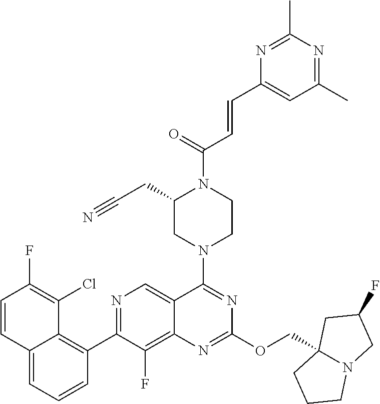 [SMILES: Cc1cc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)nc(C)n1] | | 89 | 89 | ++++ | ++++ | 3.99 | 0.03 |
| 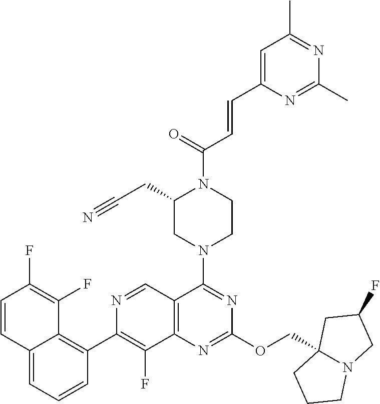 [SMILES: Cc1cc(/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)nc(C)n1] | | 90 | 90 | +++ | ++++ | 3.77 | 5.26 |
| 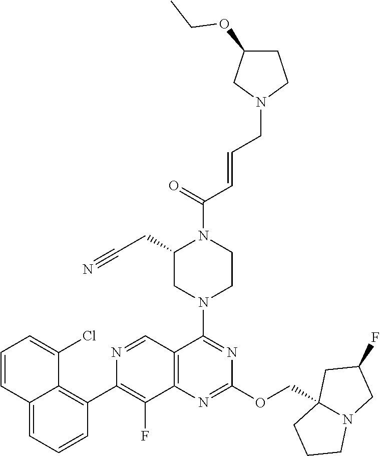 [SMILES: CCO[C@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1] | | 91 | 91 | ++++ | ++++ | 1.83 | 110.08 |
| 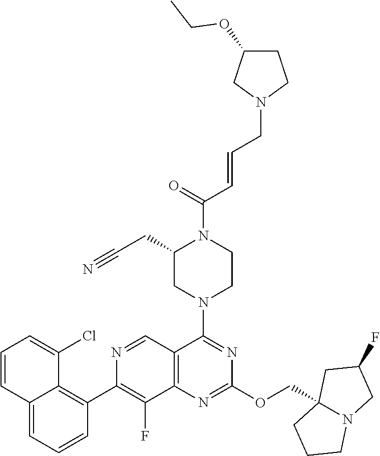 [SMILES: CCO[C@@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1] | | 92 | 92 | ++++ | ++++ | 3.35 | 104.37 |
| 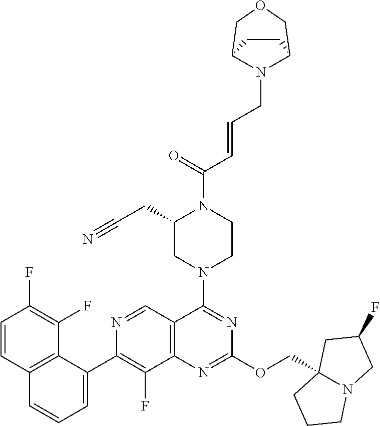 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1[C@@H]2CC[C@H]1COC2] | | 93 | 93 | ++++ | ++++ | 3.07 | 72.42 |
| 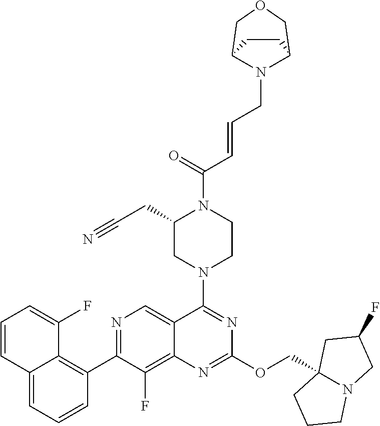 [SMILES: N#CC[C@H]1CN(c2nc(OC[C@@]34CCCN3C[C@H](F)C4)nc3c(F)c(-c4cccc5cccc(F)c45)ncc23)CCN1C(=O)/C=C/CN1[C@@H]2CC[C@H]1COC2] | | 94 | 94 | ++++ | ++++ | 3.11 | 90.24 |
| 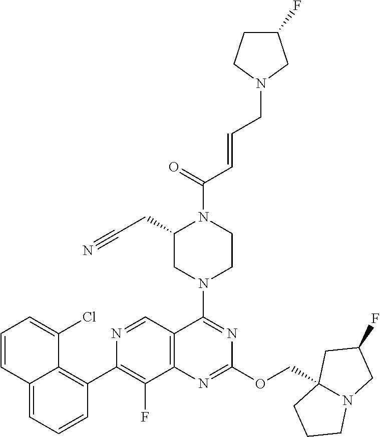 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@H](F)C1] | | 95 | 95 | ++++ | ++++ | 1.63 | 73.57 |
| 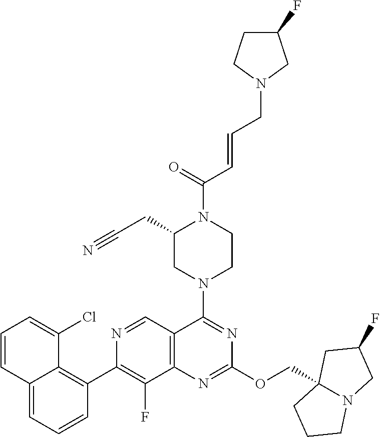 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@@H](F)C1] | | 96 | 96 | ++++ | ++++ | 2.82 | 64.43 |
| 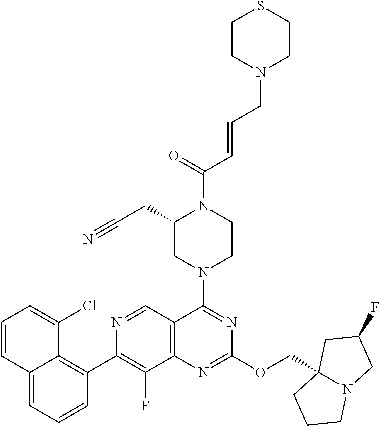 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCSCC1] | | 97 | 97 | ++++ | ++++ | 3.65 | 18.92 |
| 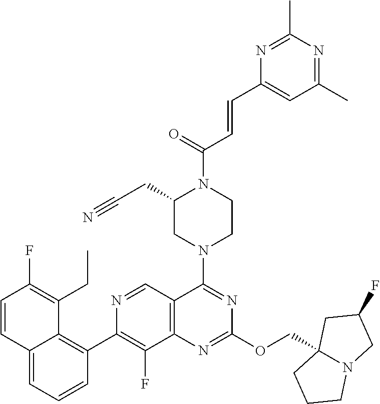 [SMILES: CCc1c(F)ccc2cccc(-c3ncc4c(N5CCN(C(=O)/C=C/c6cc(C)nc(C)n6)[C@@H](CC#N)C5)nc(OC[C@@]56CCCN5C[C@H](F)C6)nc4c3F)c12] | | 98 | 98 | ++++ | | 4.25 | 0.67 |
| 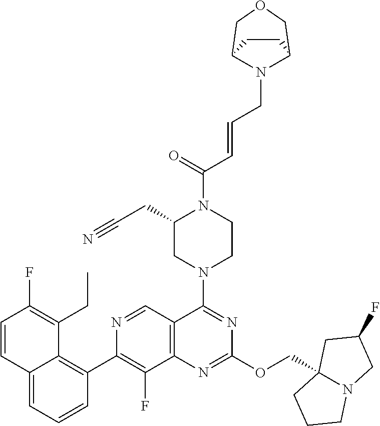 [SMILES: CCc1c(F)ccc2cccc(-c3ncc4c(N5CCN(C(=O)/C=C/CN6[C@@H]7CC[C@H]6COC7)[C@@H](CC#N)C5)nc(OC[C@@]56CCCN5C[C@H](F)C6)nc4c3F)c12] | | 99 | 99 | ++++ | | 3.40 | 77.97 |
| 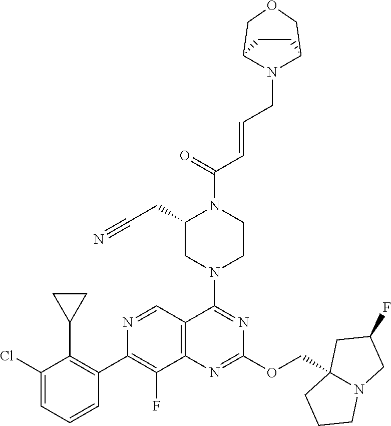 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1[C@@H]2CC[C@H]1COC2] | | 100 | 100 | ++++ | ++++ | 1.36 | 132.38 |
| 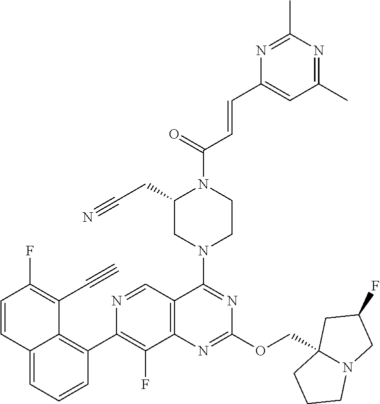 [SMILES: C#Cc1c(F)ccc2cccc(-c3ncc4c(N5CCN(C(=O)/C=C/c6cc(C)nc(C)n6)[C@@H](CC#N)C5)nc(OC[C@@]56CCCN5C[C@H](F)C6)nc4c3F)c12] | | 101 | 101 | ++++ | ++++ | 3.32 | 1.49 |
| 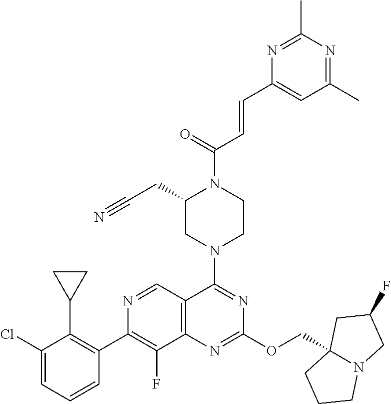 [SMILES: Cc1cc(/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)nc(C)n1] | | 102 | 102 | ++++ | ++++ | 3.98 | 11.97 |
| 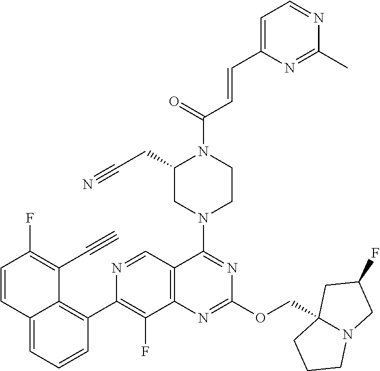 [SMILES: C#Cc1c(F)ccc2cccc(-c3ncc4c(N5CCN(C(=O)/C=C/c6ccnc(C)n6)[C@@H](CC#N)C5)nc(OC[C@@]56CCCN5C[C@H](F)C6)nc4c3F)c12] | | 103 | 103 | ++++ | ++++ | 3.29 | 10.03 |
| 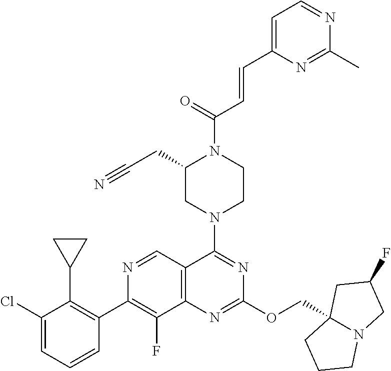 [SMILES: Cc1nccc(/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)n1] | | 104 | 104 | ++++ | ++++ | 3.60 | 15.33 |
| 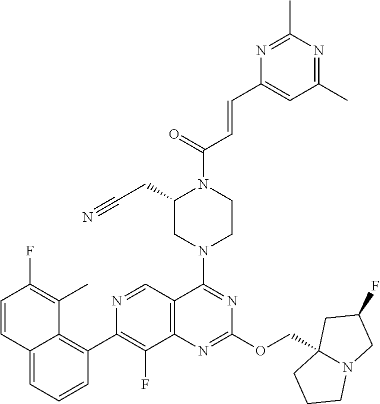 [SMILES: Cc1cc(/C=C/C(=O)N2CCN(c3nc(OC[C@@]45CCCN4C[C@H](F)C5)nc4c(F)c(-c5cccc6ccc(F)c(C)c56)ncc34)C[C@@H]2CC#N)nc(C)n1] | | 105 | 105 | ++++ | | 3.99 | 2.36 |
| 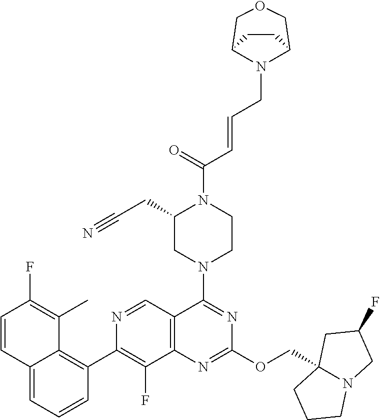 [SMILES: Cc1c(F)ccc2cccc(-c3ncc4c(N5CCN(C(=O)/C=C/CN6[C@@H]7CC[C@H]6COC7)[C@@H](CC#N)C5)nc(OC[C@@]56CCCN5C[C@H](F)C6)nc4c3F)c12] | | 106 | 106 | ++++ | | | |
| 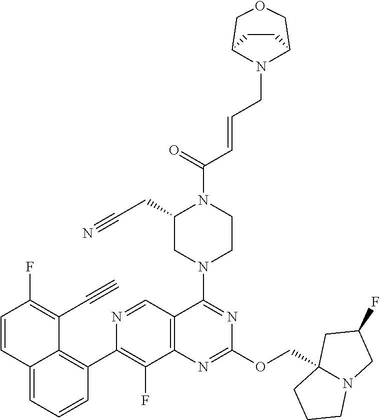 [SMILES: C#Cc1c(F)ccc2cccc(-c3ncc4c(N5CCN(C(=O)/C=C/CN6[C@@H]7CC[C@H]6COC7)[C@@H](CC#N)C5)nc(OC[C@@]56CCCN5C[C@H](F)C6)nc4c3F)c12] | | 107 | 107 | ++++ | | 2.93 | 132.33 |
| 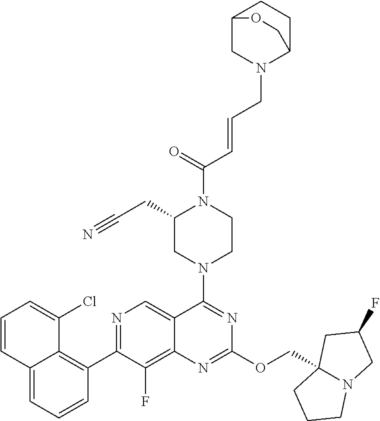 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CCC1CO2] | | 108 | 108 | ++++ | | 3.00 | 87.96 |
| 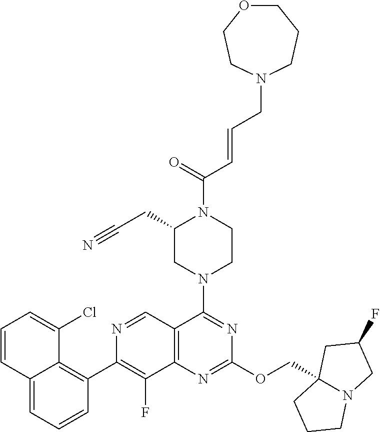 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCCOCC1] | | 109 | 109 | ++++ | | 2.60 | 126.75 |
| 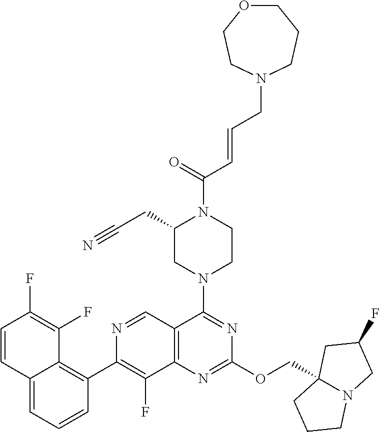 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCCOCC1] formate salt | | 110 | 110 | ++++ | | 2.90 | 78.61 |
| 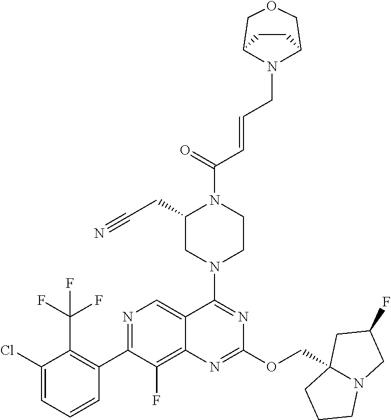 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1[C@@H]2CC[C@H]1COC2] | | 111 | 111 | ++++ | ++++ | 3.23 | 132.84 |
| 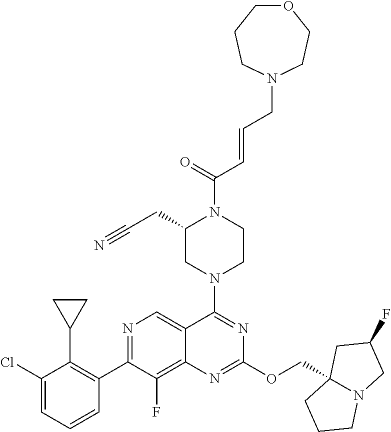 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CCCOCC1] | | 112 | 112 | ++++ | | 3.28 | 52.39 |
| 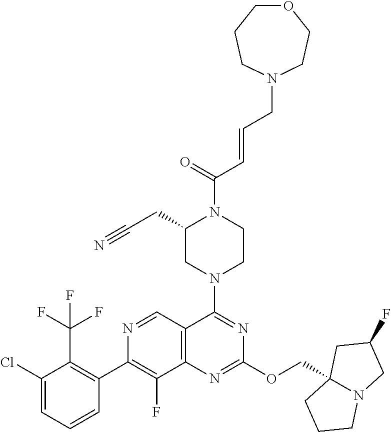 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CCCOCC1] | | 113 | 113 | ++++ | ++++ | 2.84 | 123.66 |
| 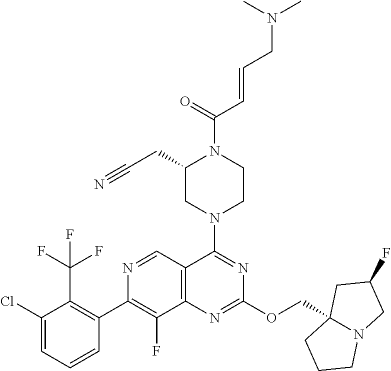 [SMILES: CN(C)C/C=C/C(=O)N1CCN(c2nc(OC[C@@]34CCCN3C[C@H](F)C4)nc3c(F)c(-c4cccc(Cl)c4C(F)(F)F)ncc23)C[C@@H]1CC#N] | | 114 | 114 | ++++ | | 2.35 | 120.52 |
| 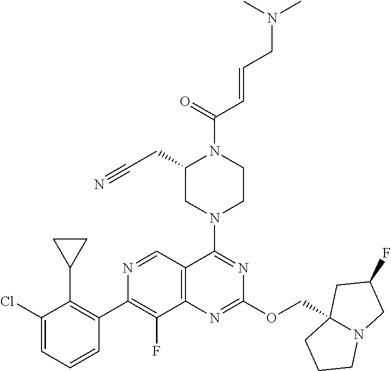 [SMILES: CN(C)C/C=C/C(=O)N1CCN(c2nc(OC[C@@]34CCCN3C[C@H](F)C4)nc3c(F)c(-c4cccc(Cl)c4C4CC4)ncc23)C[C@@H]1CC#N] | | 115 | 115 | +++ | | 2.54 | 114.02 |
| 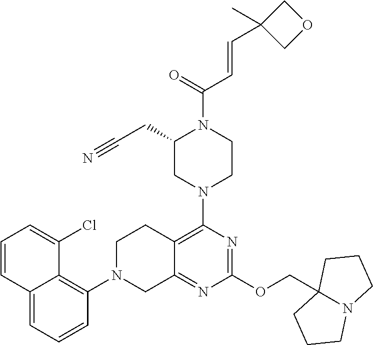 [SMILES: CC1(/C=C/C(=O)N2CCN(c3nc(OCC45CCCN4CCC5)nc4c3CCN(c3cccc5cccc(Cl)c35)C4)C[C@@H]2CC#N)COC1] | | 116 | | + | | | |
| 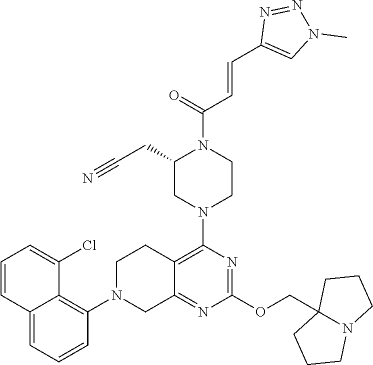 [SMILES: Cn1cc(/C=C/C(=O)N2CCN(c3nc(OCC45CCCN4CCC5)nc4c3CCN(c3cccc5cccc(Cl)c35)C4)C[C@@H]2CC#N)nn1] | | 117 | | + | | 2.65 | 111.94 |
| 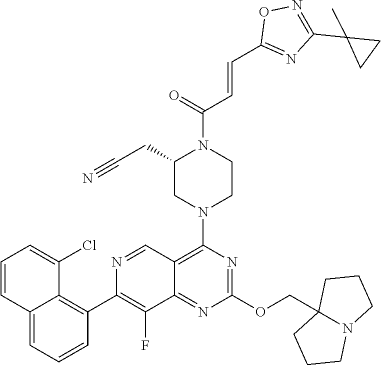 [SMILES: CC1(c2noc(/C=C/C(=O)N3CCN(c4nc(OCC56CCCN5CCC6)nc5c(F)c(-c6cccc7cccc(Cl)c67)ncc45)C[C@@H]3CC#N)n2)CC1] | | 118 | | ++ | | 3.24 | 4.73 |
| 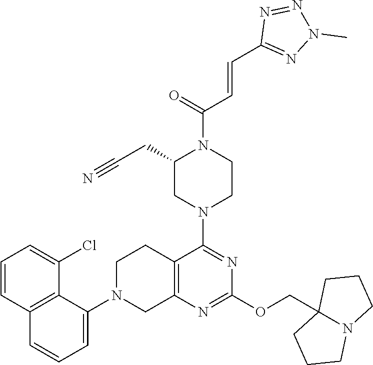 [SMILES: Cn1nnc(/C=C/C(=O)N2CCN(c3nc(OCC45CCCN4CCC5)nc4c3CCN(c3cccc5cccc(Cl)c35)C4)C[C@@H]2CC#N)n1] | | 119 | | + | | | |
| 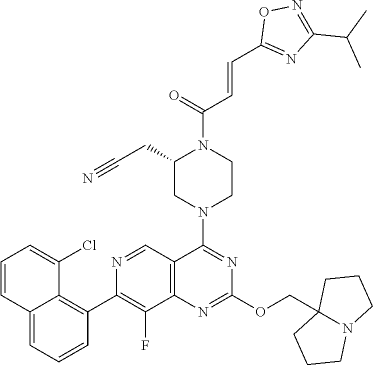 [SMILES: CC(C)c1noc(/C=C/C(=O)N2CCN(c3nc(OCC45CCCN4CCC5)nc4c(F)c(-c5cccc6cccc(Cl)c56)ncc34)C[C@@H]2CC#N)n1] | | 120 | | ++ | | 3.21 | 26.49 |
| 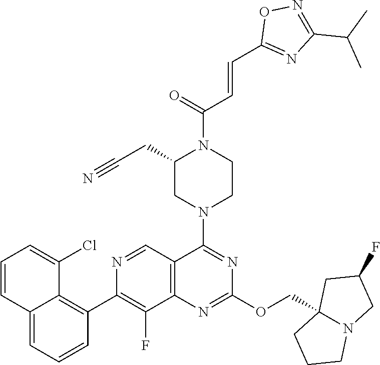 [SMILES: CC(C)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1] | | 121 | | ++ | | 4.28 | 0.01 |
| 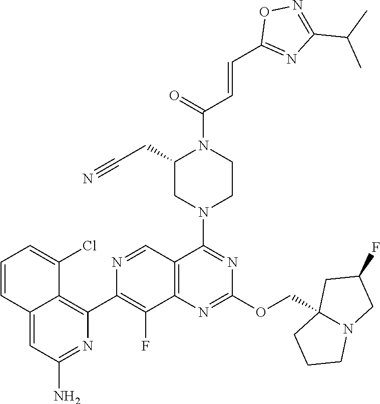 [SMILES: CC(C)c1noc(/C=C/C(=O)N2CCN(c3nc(OC[C@@]45CCCN4C[C@H](F)C5)nc4c(F)c(-c5nc(N)cc6cccc(Cl)c56)ncc34)C[C@@H]2CC#N)n1] | | 122 | | +++ | | 3.43 | 0.44 |
| 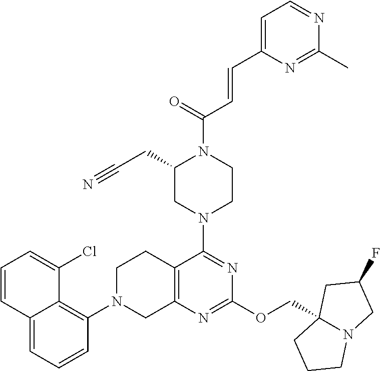 [SMILES: Cc1nccc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1] | | 123 | | ++++ | ++++ | 4.46 | 0.01 |
| 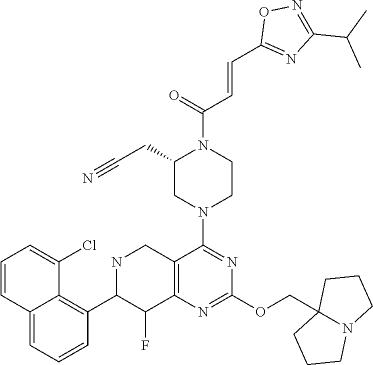 [SMILES: CC(C)c1noc(/C=C/C(=O)N2CCN(c3nc(OCC45CCCN4CCC5)nc4c3CNC(c3cccc5cccc(Cl)c35)C4F)C[C@@H]2CC#N)n1] | | 124 | | +++ | +++ | 4.21 | 0.23 |
| 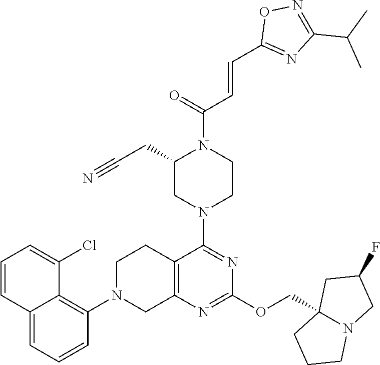 [SMILES: CC(C)c1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1] | | 125 | | +++ | | 3.41 | 0.01 |
| 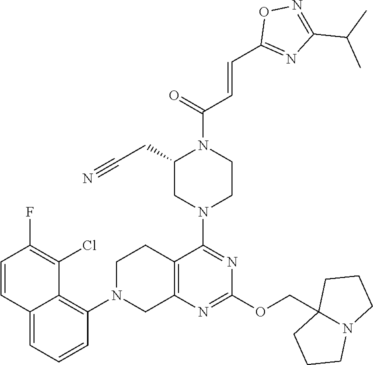 [SMILES: CC(C)c1noc(/C=C/C(=O)N2CCN(c3nc(OCC45CCCN4CCC5)nc4c3CCN(c3cccc5ccc(F)c(Cl)c35)C4)C[C@@H]2CC#N)n1] | | 126 | | +++ | +++ | 4.24 | 0.01 |
| 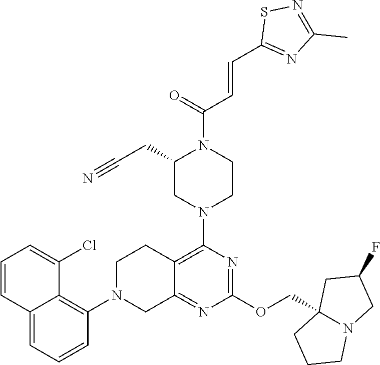 [SMILES: Cc1nsc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1] | | 127 | | ++++ | ++++ | 4.77 | 0.01 |
| 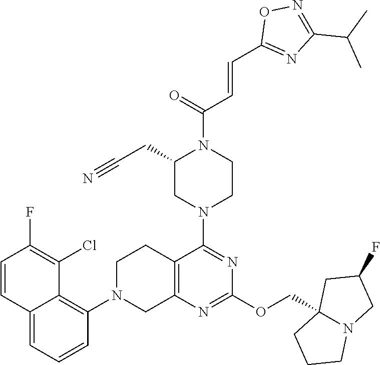 [SMILES: CC(C)c1noc(/C=C/C(=O)N2CCN(c3nc(OC[C@@]45CCCN4C[C@H](F)C5)nc4c3CCN(c3cccc5ccc(F)c(Cl)c35)C4)C[C@@H]2CC#N)n1] | | 128 | | +++ | +++ | 4.56 | 0.01 |
| 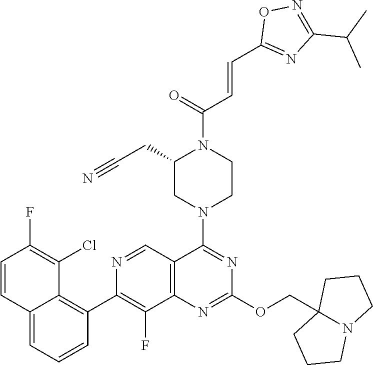 [SMILES: CC(C)c1noc(/C=C/C(=O)N2CCN(c3nc(OCC45CCCN4CCC5)nc4c(F)c(-c5cccc6ccc(F)c(Cl)c56)ncc34)C[C@@H]2CC#N)n1] | | 129 | | ++ | | 3.35 | 46.91 |
| 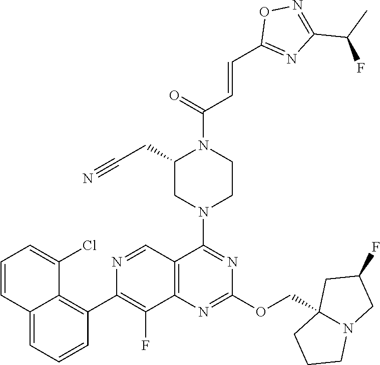 [SMILES: C[C@@H](F)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1] | | 130 | | +++ | | 3.99 | 0.04 |
| 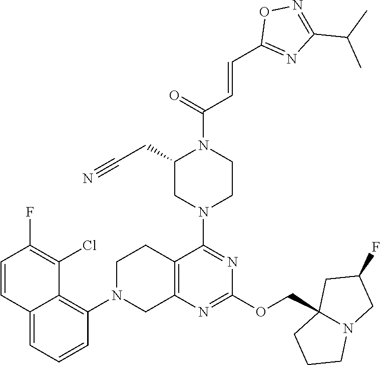 [SMILES: CC(C)c1noc(/C=C/C(=O)N2CCN(c3nc(OC[C@]45CCCN4C[C@H](F)C5)nc4c3CCN(c3cccc5ccc(F)c(Cl)c35)C4)C[C@@H]2CC#N)n1] | | 131 | | +++ | +++ | 4.96 | 0.01 |
| 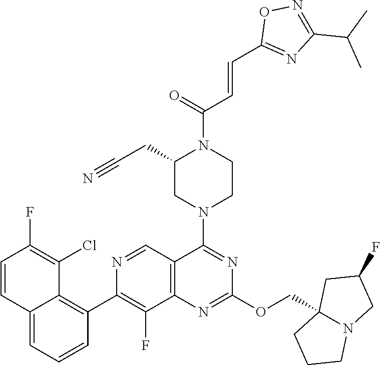 [SMILES: CC(C)c1noc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1] | | 132 | | +++ | | 4.49 | 0.01 |
| 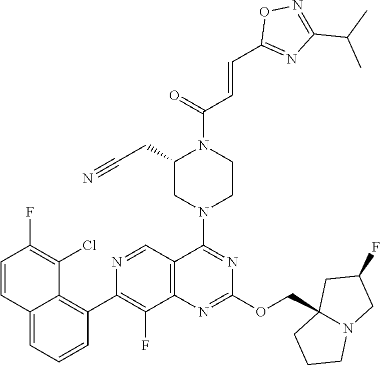 [SMILES: CC(C)c1noc(/C=C/C(=O)N2CCN(c3nc(OC[C@]45CCCN4C[C@H](F)C5)nc4c(F)c(-c5cccc6ccc(F)c(Cl)c56)ncc34)C[C@@H]2CC#N)n1] | | 133 | | +++ | | 4.35 | 0.02 |
| 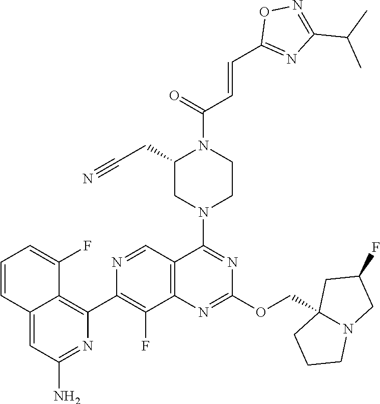 [SMILES: CC(C)c1noc(/C=C/C(=O)N2CCN(c3nc(OC[C@@]45CCCN4C[C@H](F)C5)nc4c(F)c(-c5nc(N)cc6cccc(F)c56)ncc34)C[C@@H]2CC#N)n1] | | 134 | | +++ | | 3.36 | 20.43 |
| 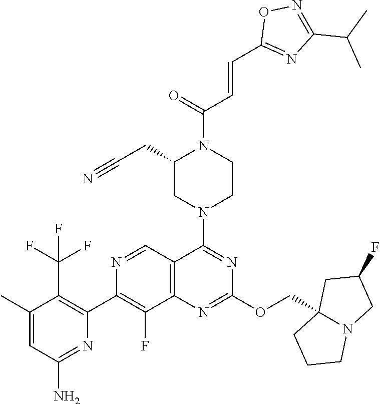 [SMILES: Cc1cc(N)nc(-c2ncc3c(N4CCN(C(=O)/C=C/c5nc(C(C)C)no5)[C@@H](CC#N)C4)nc(OC[C@@]45CCCN4C[C@H](F)C5)nc3c2F)c1C(F)(F)F] | | 135 | | +++ | | 3.42 | 94.59 |
| 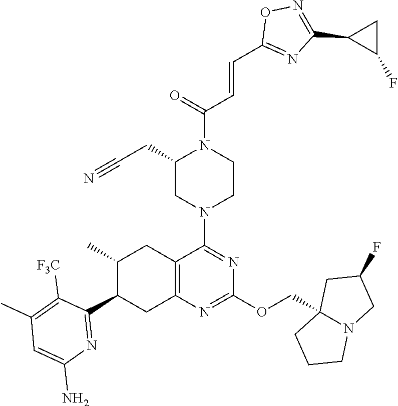 [SMILES: Cc1cc(N)nc([C@@H]2Cc3nc(OC[C@@]45CCCN4C[C@H](F)C5)nc(N4CCN(C(=O)/C=C/c5nc([C@H]6C[C@@H]6F)no5)[C@@H](CC#N)C4)c3C[C@H]2C)c1C(F)(F)F] | (and) cyclohexenyl centers, (and) cyclopropyl centers (abs) piperazine, (abs) pyrrolizine | 136 | | ++++ | ++++ | 4.37 | 0.02 |
| 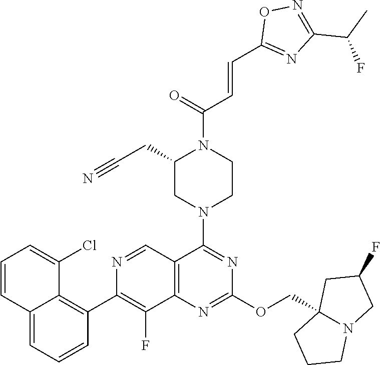 [SMILES: C[C@H](F)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1] | | 137 | | +++ | | 4.07 | 0.06 |
| 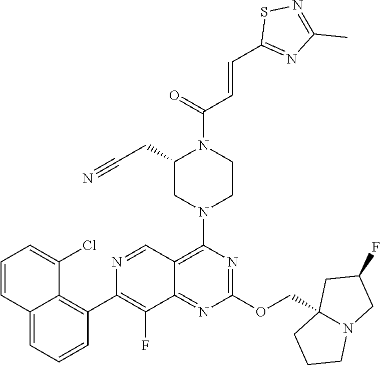 [SMILES: Cc1nsc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1] | | 138 | | ++++ | ++++ | 3.90 | 0.01 |
| 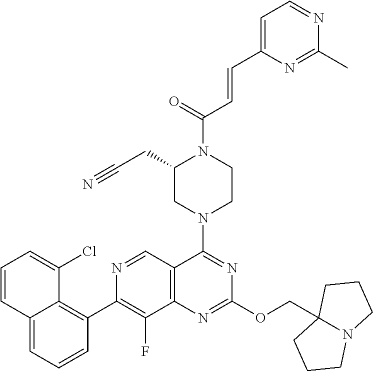 [SMILES: Cc1nccc(/C=C/C(=O)N2CCN(c3nc(OCC45CCCN4CCC5)nc4c(F)c(-c5cccc6cccc(Cl)c56)ncc34)C[C@@H]2CC#N)n1] | | 139 | | +++ | ++++ | 1.93 | 107.48 |
| 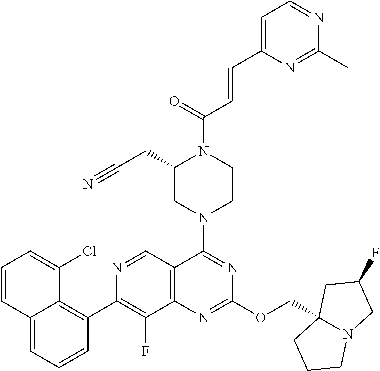 [SMILES: Cc1nccc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1] | | 140 | | ++++ | ++++ | 3.45 | 1.86 |
| 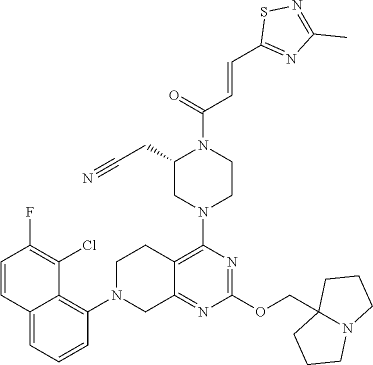 [SMILES: Cc1nsc(/C=C/C(=O)N2CCN(c3nc(OCC45CCCN4CCC5)nc4c3CCN(c3cccc5ccc(F)c(Cl)c35)C4)C[C@@H]2CC#N)n1] | | 141 | | ++++ | ++++ | 3.43 | 84.03 |
| 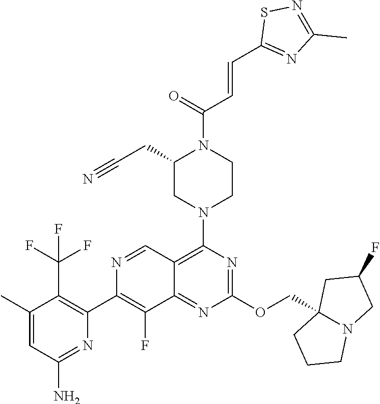 [SMILES: Cc1nsc(/C=C/C(=O)N2CCN(c3nc(OC[C@@]45CCCN4C[C@H](F)C5)nc4c(F)c(-c5nc(N)cc(C)c5C(F)(F)F)ncc34)C[C@@H]2CC#N)n1] | | 142 | | ++++ | | 2.55 | 105.63 |
| 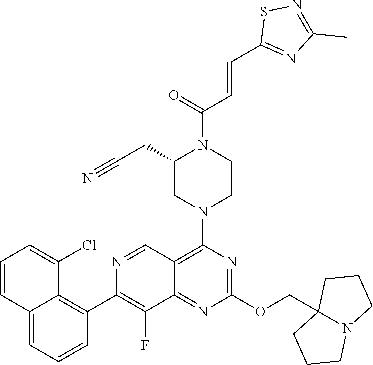 [SMILES: Cc1nsc(/C=C/C(=O)N2CCN(c3nc(OCC45CCCN4CCC5)nc4c(F)c(-c5cccc6cccc(Cl)c56)ncc34)C[C@@H]2CC#N)n1] | | 143 | | ++++ | ++++ | 2.39 | 87.83 |
| 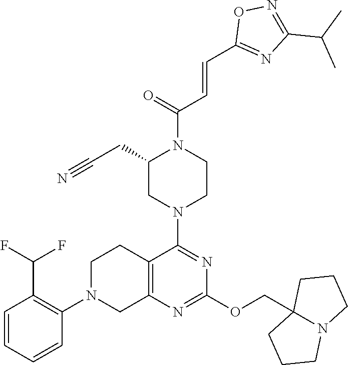 [SMILES: CC(C)c1noc(/C=C/C(=O)N2CCN(c3nc(OCC45CCCN4CCC5)nc4c3CCN(c3ccccc3C(F)F)C4)C[C@@H]2CC#N)n1] | | 144 | | + | | | |
| 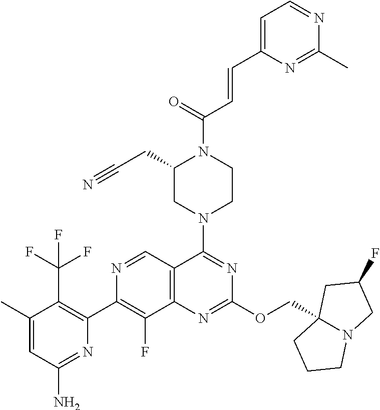 [SMILES: Cc1nccc(/C=C/C(=O)N2CCN(c3nc(OC[C@@]45CCCN4C[C@H](F)C5)nc4c(F)c(-c5nc(N)cc(C)c5C(F)(F)F)ncc34)C[C@@H]2CC#N)n1] | | 145 | | ++++ | | 2.11 | 135.79 |
| 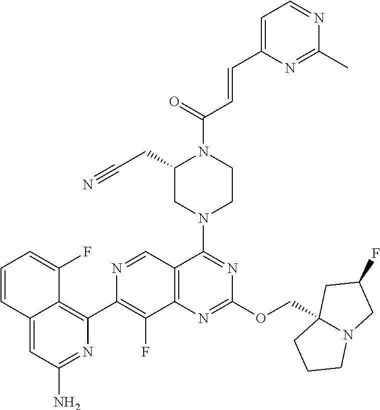 [SMILES: Cc1nccc(/C=C/C(=O)N2CCN(c3nc(OC[C@@]45CCCN4C[C@H](F)C5)nc4c(F)c(-c5nc(N)cc6cccc(F)c56)ncc34)C[C@@H]2CC#N)n1] | | 146 | | ++++ | | 2.14 | 78.14 |
| 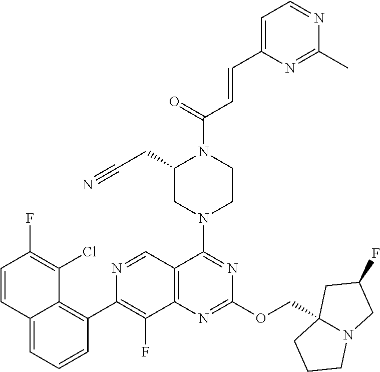 [SMILES: Cc1nccc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1] | | 147 | | ++++ | ++++ | 3.64 | 4.93 |
| 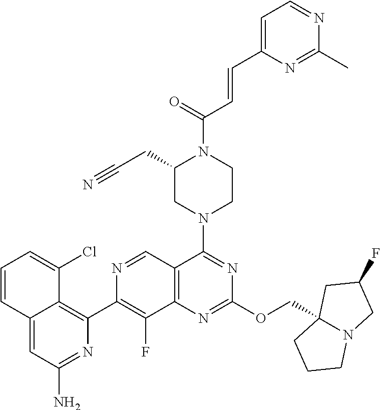 [SMILES: Cc1nccc(/C=C/C(=O)N2CCN(c3nc(OC[C@@]45CCCN4C[C@H](F)C5)nc4c(F)c(-c5nc(N)cc6cccc(Cl)c56)ncc34)C[C@@H]2CC#N)n1] | | 148 | | ++++ | ++++ | 2.25 | 83.85 |
| 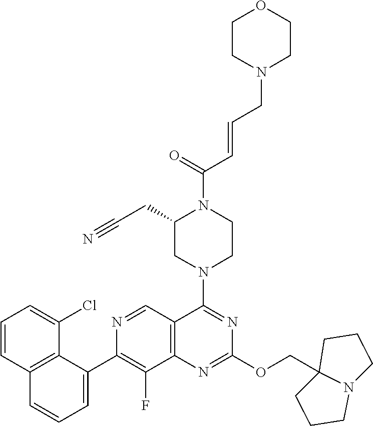 [SMILES: N#CC[C@H]1CN(c2nc(OCC34CCCN3CCC4)nc3c(F)c(-c4cccc5cccc(Cl)c45)ncc23)CCN1C(=O)/C=C/CN1CCOCC1] | | 149 | | +++ | | | 106.44 |
| 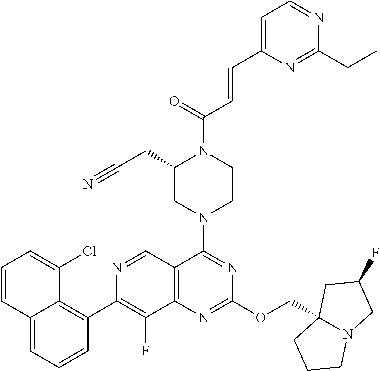 [SMILES: CCc1nccc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1] | | 150 | | ++++ | ++++ | 4.04 | 3.87 |
| 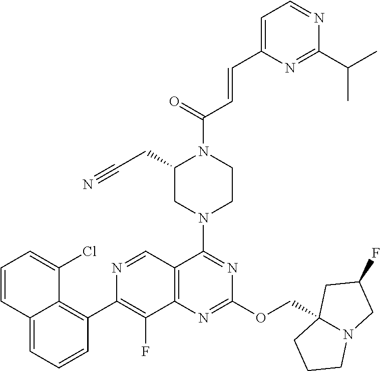 [SMILES: CC(C)c1nccc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1] | | 151 | | ++++ | ++++ | 3.71 | 0.41 |
| 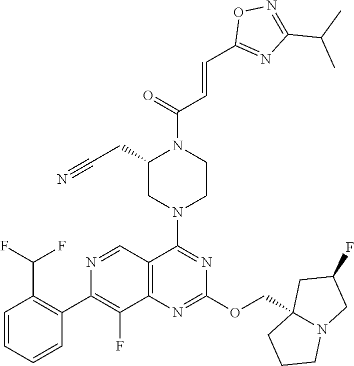 [SMILES: CC(C)c1noc(/C=C/C(=O)N2CCN(c3nc(OC[C@@]45CCCN4C[C@H](F)C5)nc4c(F)c(-c5ccccc5C(F)F)ncc34)C[C@@H]2CC#N)n1] | | 152 | | + | | 2.62 | 125.67 |
| 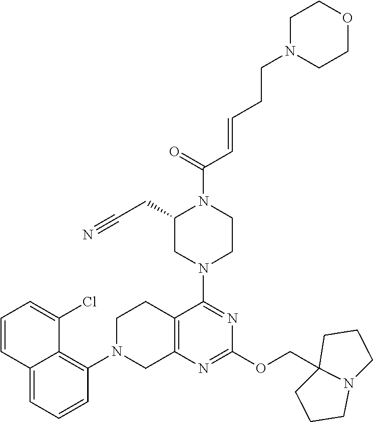 [SMILES: N#CC[C@H]1CN(c2nc(OCC34CCCN3CCC4)nc3c2CCN(c2cccc4cccc(Cl)c24)C3)CCN1C(=O)/C=C/CCN1CCOCC1] | | 153 | | +++ | | | |
| 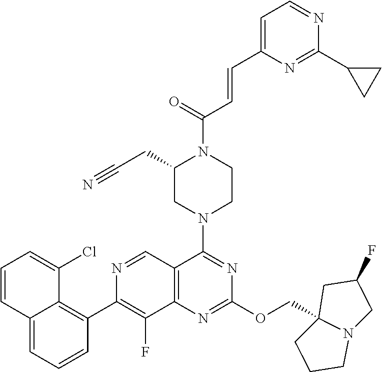 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/c1ccnc(C2CC2)n1] | | 154 | | ++++ | ++++ | 4.36 | 0.01 |
| 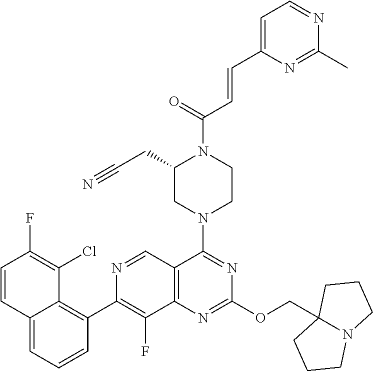 [SMILES: Cc1nccc(/C=C/C(=O)N2CCN(c3nc(OCC45CCCN4CCC5)nc4c(F)c(-c5cccc6ccc(F)c(Cl)c56)ncc34)C[C@@H]2CC#N)n1] | | 155 | | ++++ | | | 89.11 |
| 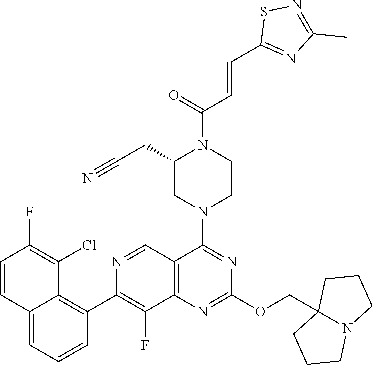 [SMILES: Cc1nsc(/C=C/C(=O)N2CCN(c3nc(OCC45CCCN4CCC5)nc4c(F)c(-c5cccc6ccc(F)c(Cl)c56)ncc34)C[C@@H]2CC#N)n1] | | 156 | | ++++ | ++++ | 2.78 | 84.45 |
| 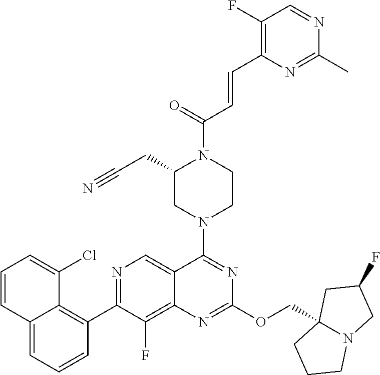 [SMILES: Cc1ncc(F)c(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1] | | 157 | | +++ | | 4.05 | 0.55 |
| 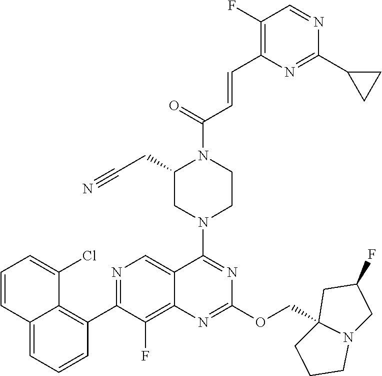 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/c1nc(C2CC2)ncc1F] | | 158 | | ++ | | 4.52 | 0.01 |
| 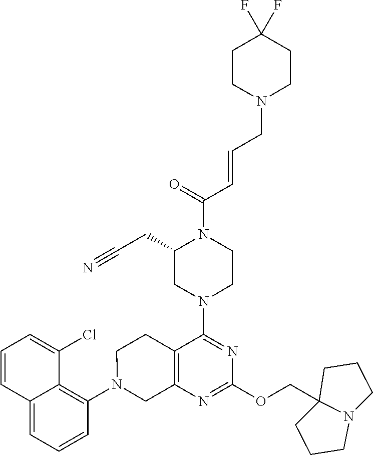 [SMILES: N#CC[C@H]1CN(c2nc(OCC34CCCN3CCC4)nc3c2CCN(c2cccc4cccc(Cl)c24)C3)CCN1C(=O)/C=C/CN1CCC(F)(F)CC1] | | 159 | | ++++ | ++++ | | |
| 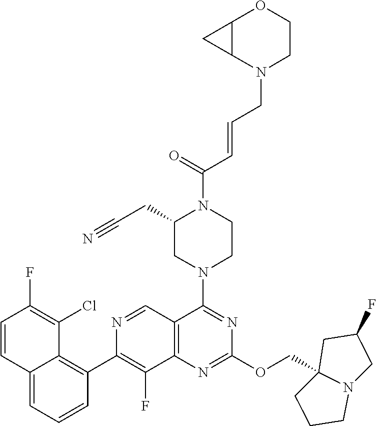 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCOC2CC21] | | 160 | | ++++ | | 3.33 | 86.83 |
| 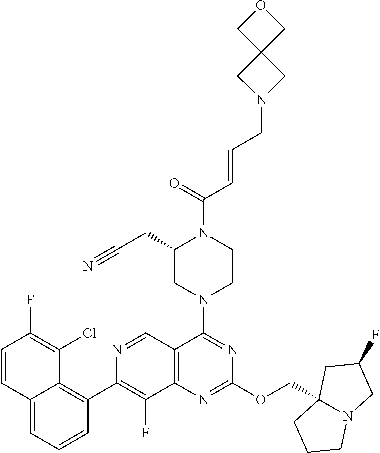 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2(COC2)C1] | | 161 | | ++++ | ++++ | 2.75 | 87.74 |
| 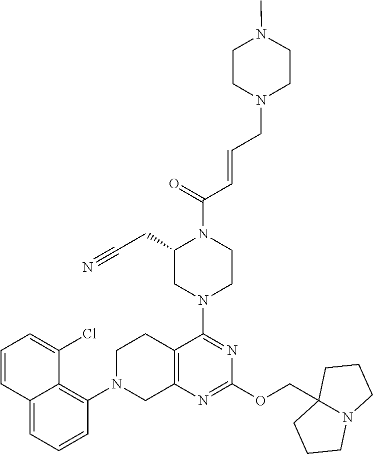 [SMILES: CN1CCN(C/C=C/C(=O)N2CCN(c3nc(OCC45CCCN4CCC5)nc4c3CCN(c3cccc5cccc(Cl)c35)C4)C[C@@H]2CC#N)CC1] | | 162 | | +++ | | 1.97 | 132.67 |
| 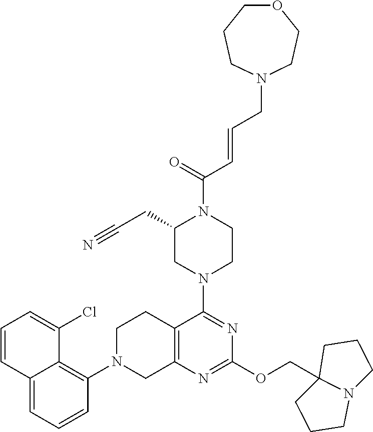 [SMILES: N#CC[C@H]1CN(c2nc(OCC34CCCN3CCC4)nc3c2CCN(c2cccc4cccc(Cl)c24)C3)CCN1C(=O)/C=C/CN1CCCOCC1] | | 163 | | ++++ | | 2.44 | 130.28 |
| 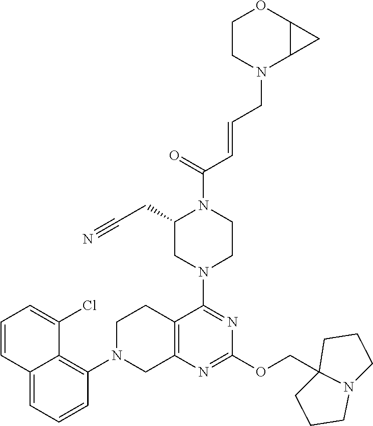 [SMILES: N#CC[C@H]1CN(c2nc(OCC34CCCN3CCC4)nc3c2CCN(c2cccc4cccc(Cl)c24)C3)CCN1C(=O)/C=C/CN1CCOC2CC21] | | 164 | | +++ | | | |
| 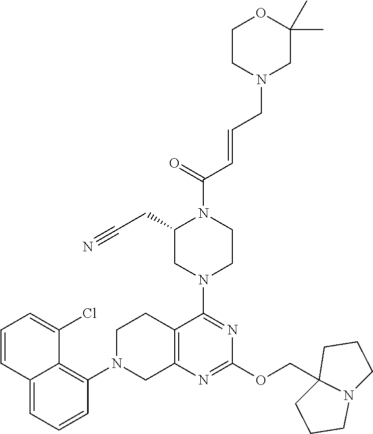 [SMILES: CC1(C)CN(C/C=C/C(=O)N2CCN(c3nc(OCC45CCCN4CCC5)nc4c3CCN(c3cccc5cccc(Cl)c35)C4)C[C@@H]2CC#N)CCO1] | | 165 | | ++++ | ++++ | | |
| 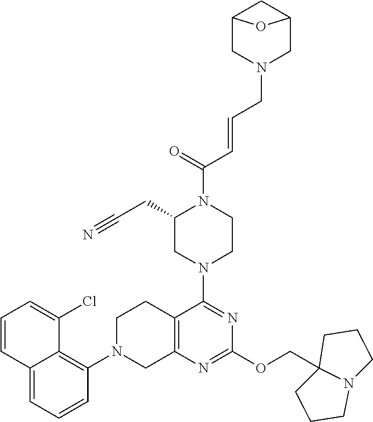 [SMILES: N#CC[C@H]1CN(c2nc(OCC34CCCN3CCC4)nc3c2CCN(c2cccc4cccc(Cl)c24)C3)CCN1C(=O)/C=C/CN1CC2CC(C1)O2] | | 166 | | ++++ | | | |
| 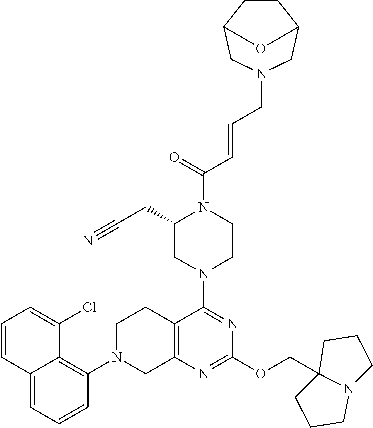 [SMILES: N#CC[C@H]1CN(c2nc(OCC34CCCN3CCC4)nc3c2CCN(c2cccc4cccc(Cl)c24)C3)CCN1C(=O)/C=C/CN1CC2CCC(C1)O2] | | 167 | | ++++ | | 3.13 | 101.30 |
| 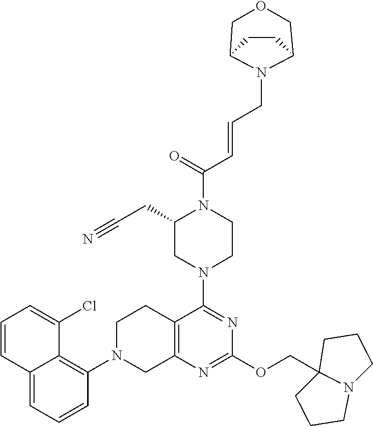 [SMILES: N#CC[C@H]1CN(c2nc(OCC34CCCN3CCC4)nc3c2CCN(c2cccc4cccc(Cl)c24)C3)CCN1C(=O)/C=C/CN1[C@@H]2CC[C@H]1COC2] | | 168 | | ++++ | | 2.59 | 104.47 |
| 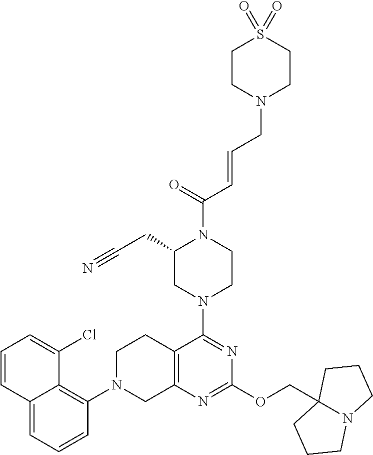 [SMILES: N#CC[C@H]1CN(c2nc(OCC34CCCN3CCC4)nc3c2CCN(c2cccc4cccc(Cl)c24)C3)CCN1C(=O)/C=C/CN1CCS(=O)(=O)CC1] | | 169 | | +++ | | 2.02 | 107.85 |
| 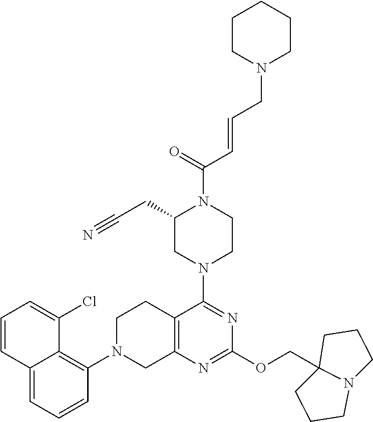 [SMILES: N#CC[C@H]1CN(c2nc(OCC34CCCN3CCC4)nc3c2CCN(c2cccc4cccc(Cl)c24)C3)CCN1C(=O)/C=C/CN1CCCCC1] | | 170 | | ++++ | ++++ | | |
| 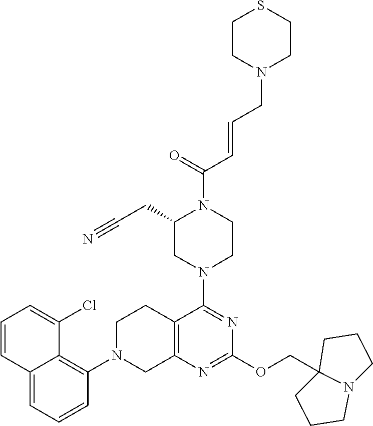 [SMILES: N#CC[C@H]1CN(c2nc(OCC34CCCN3CCC4)nc3c2CCN(c2cccc4cccc(Cl)c24)C3)CCN1C(=O)/C=C/CN1CCSCC1] | | 171 | | ++++ | ++++ | 3.30 | 108.27 |
| 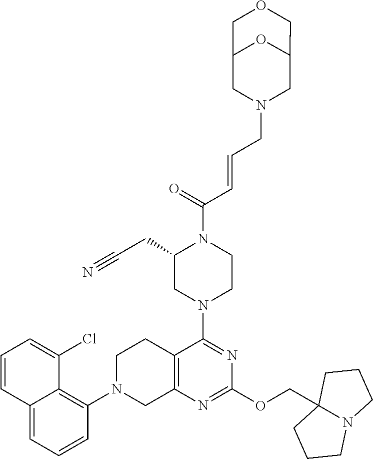 [SMILES: N#CC[C@H]1CN(c2nc(OCC34CCCN3CCC4)nc3c2CCN(c2cccc4cccc(Cl)c24)C3)CCN1C(=O)/C=C/CN1CC2COCC(C1)O2] | | 172 | | ++++ | | 2.10 | 119.37 |
| 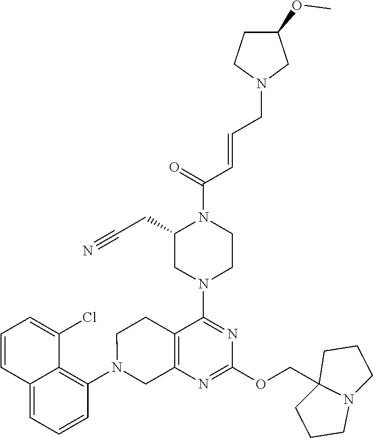 [SMILES: CO[C@@H]1CCN(C/C=C/C(=O)N2CCN(c3nc(OCC45CCCN4CCC5)nc4c3CCN(c3cccc5cccc(Cl)c35)C4)C[C@@H]2CC#N)C1] | | 173 | | ++++ | | 2.31 | 88.86 |
| 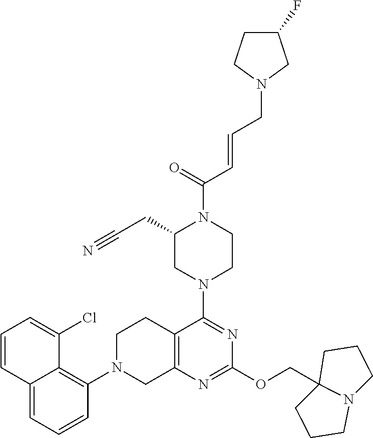 [SMILES: N#CC[C@H]1CN(c2nc(OCC34CCCN3CCC4)nc3c2CCN(c2cccc4cccc(Cl)c24)C3)CCN1C(=O)/C=C/CN1CC[C@H](F)C1] | | 174 | | ++++ | | | |
| 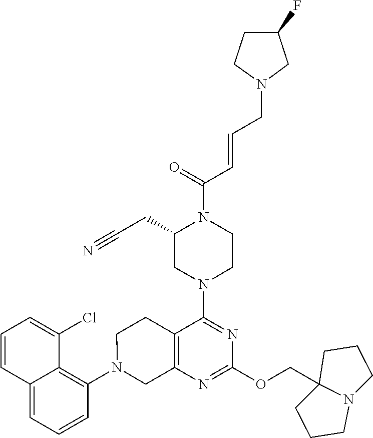 [SMILES: N#CC[C@H]1CN(c2nc(OCC34CCCN3CCC4)nc3c2CCN(c2cccc4cccc(Cl)c24)C3)CCN1C(=O)/C=C/CN1CC[C@@H](F)C1] | | 175 | | ++++ | | | |
| 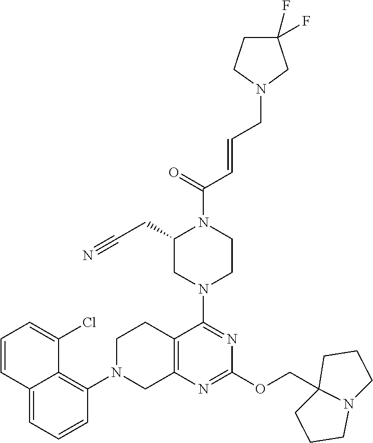 [SMILES: N#CC[C@H]1CN(c2nc(OCC34CCCN3CCC4)nc3c2CCN(c2cccc4cccc(Cl)c24)C3)CCN1C(=O)/C=C/CN1CCC(F)(F)C1] | | 176 | | +++ | | | |
| 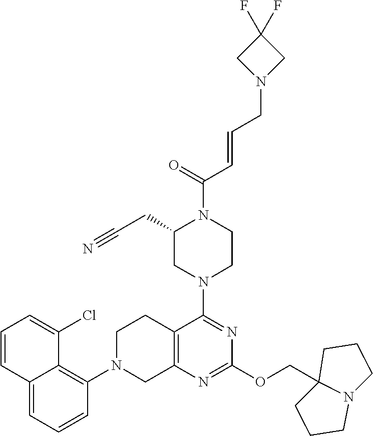 [SMILES: N#CC[C@H]1CN(c2nc(OCC34CCCN3CCC4)nc3c2CCN(c2cccc4cccc(Cl)c24)C3)CCN1C(=O)/C=C/CN1CC(F)(F)C1] | | 177 | | +++ | | | |
| 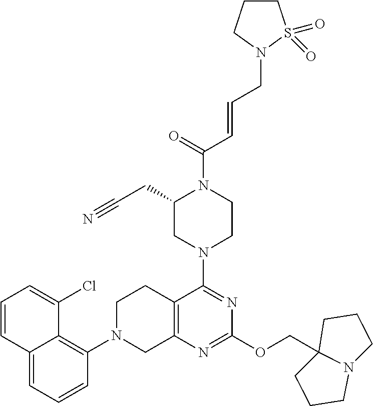 [SMILES: N#CC[C@H]1CN(c2nc(OCC34CCCN3CCC4)nc3c2CCN(c2cccc4cccc(Cl)c24)C3)CCN1C(=O)/C=C/CN1CCCS1(=O)=O] | | 178 | | ++ | | | |
| 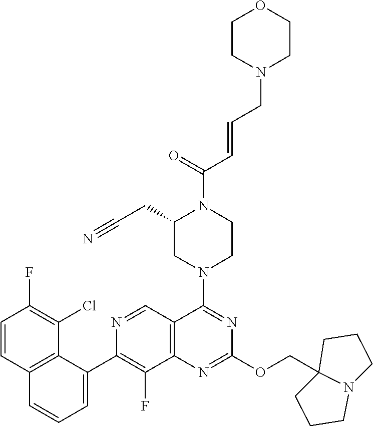 [SMILES: N#CC[C@H]1CN(c2nc(OCC34CCCN3CCC4)nc3c(F)c(-c4cccc5ccc(F)c(Cl)c45)ncc23)CCN1C(=O)/C=C/CN1CCOCC1] | | 179 | | ++++ | | 1.63 | 117.97 |
| 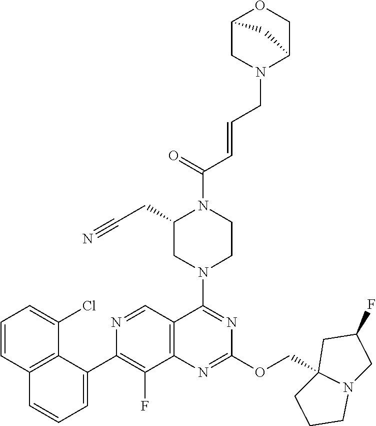 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@@H]2C[C@H]1CO2] | | 180 | | ++++ | ++++ | 2.74 | 91.33 |
| 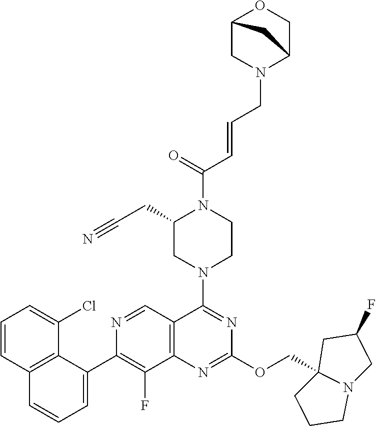 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@H]2C[C@@H]1CO2] | | 181 | | ++++ | ++++ | 2.80 | 99.60 |
| 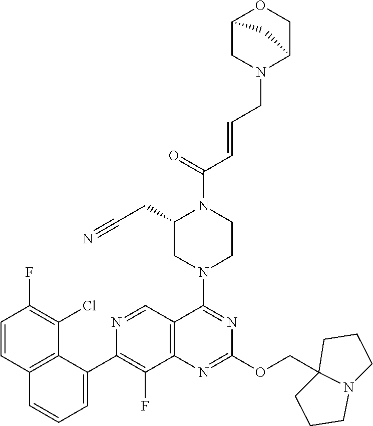 [SMILES: N#CC[C@H]1CN(c2nc(OCC34CCCN3CCC4)nc3c(F)c(-c4cccc5ccc(F)c(Cl)c45)ncc23)CCN1C(=O)/C=C/CN1C[C@@H]2C[C@H]1CO2] | | 182 | | ++++ | | 1.44 | 119.05 |
| 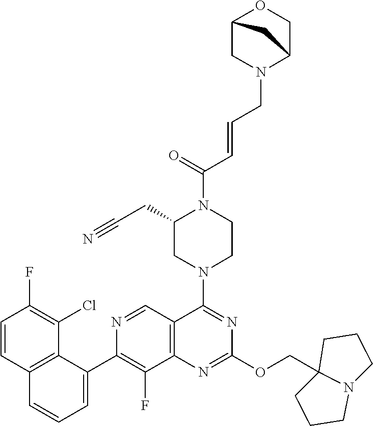 [SMILES: N#CC[C@H]1CN(c2nc(OCC34CCCN3CCC4)nc3c(F)c(-c4cccc5ccc(F)c(Cl)c45)ncc23)CCN1C(=O)/C=C/CN1C[C@H]2C[C@@H]1CO2] | | 183 | | ++++ | | 1.48 | 110.38 |
| 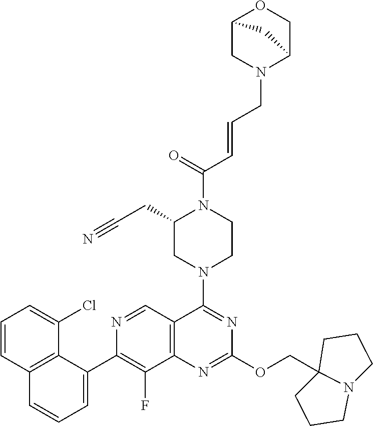 [SMILES: N#CC[C@H]1CN(c2nc(OCC34CCCN3CCC4)nc3c(F)c(-c4cccc5cccc(Cl)c45)ncc23)CCN1C(=O)/C=C/CN1C[C@@H]2C[C@H]1CO2] | | 184 | | ++++ | | 1.36 | 103.63 |
| 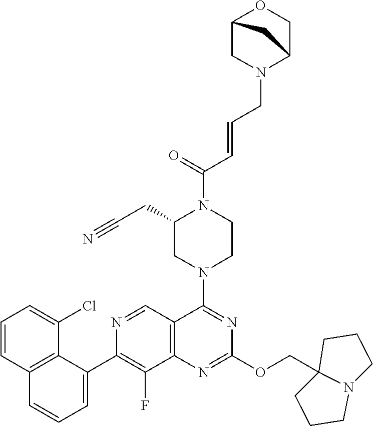 [SMILES: N#CC[C@H]1CN(c2nc(OCC34CCCN3CCC4)nc3c(F)c(-c4cccc5cccc(Cl)c45)ncc23)CCN1C(=O)/C=C/CN1C[C@H]2C[C@@H]1CO2] | | 185 | | ++++ | | 1.45 | 104.23 |
| 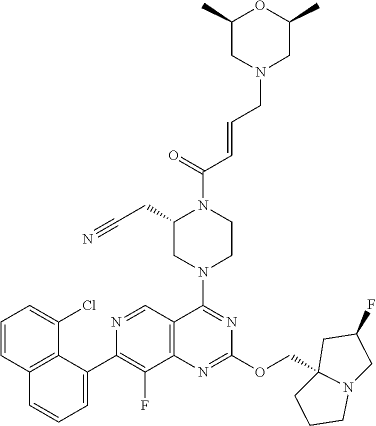 [SMILES: C[C@@H]1CN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C[C@H](C)O1] | | 186 | | +++ | +++ | 3.54 | 89.01 |
| 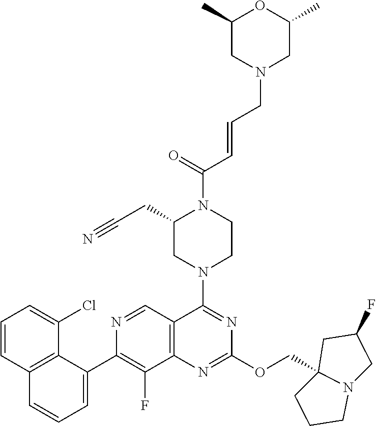 [SMILES: C[C@@H]1CN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C[C@@H](C)O1] | | 187 | | ++++ | ++++ | 3.61 | 83.98 |
| 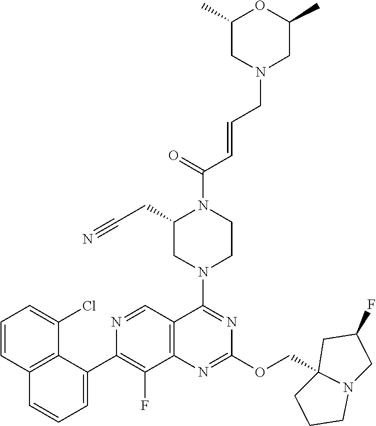 [SMILES: C[C@H]1CN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C[C@H](C)O1] | | 188 | | ++++ | ++++ | 3.86 | 79.10 |
| 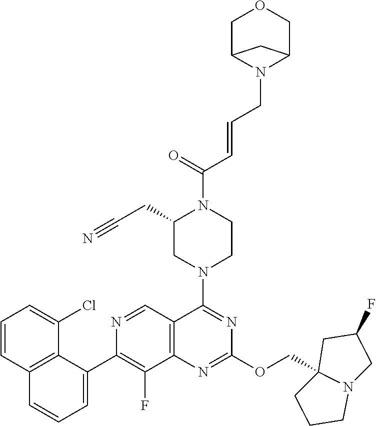 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C2COCC1C2] | | 189 | | ++++ | ++++ | 2.96 | 108.97 |
| 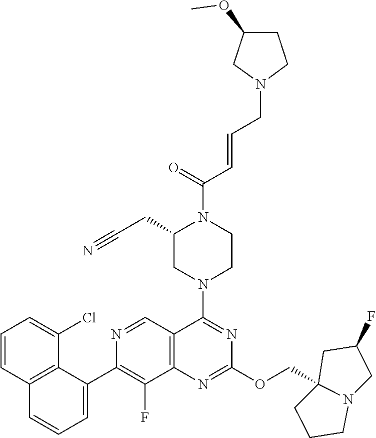 [SMILES: CO[C@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1] | | 190 | | ++++ | ++++ | 2.86 | 94.66 |
| 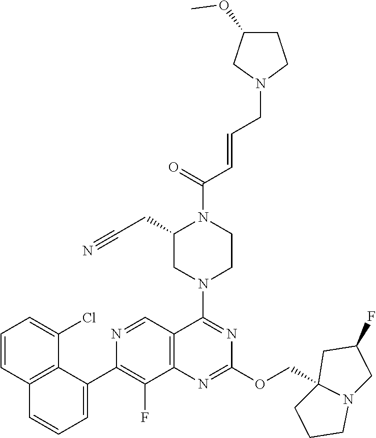 [SMILES: CO[C@@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1] | | 191 | | ++++ | ++++ | 3.18 | 94.89 |
| 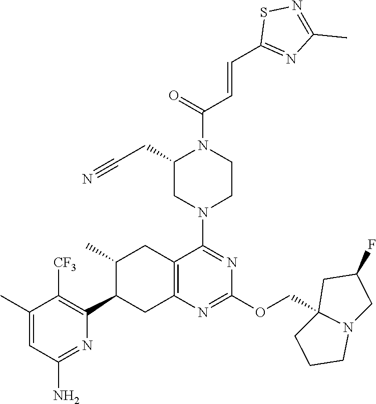 [SMILES: Cc1nsc(/C=C/C(=O)N2CCN(c3nc(OC[C@@]45CCCN4C[C@H](F)C5)nc4c3C[C@@H](C)[C@H](c3nc(N)cc(C)c3C(F)(F)F)C4)C[C@@H]2CC#N)n1] | | 192 | | ++++ | ++++ | 4.69 | 2.01 |
| 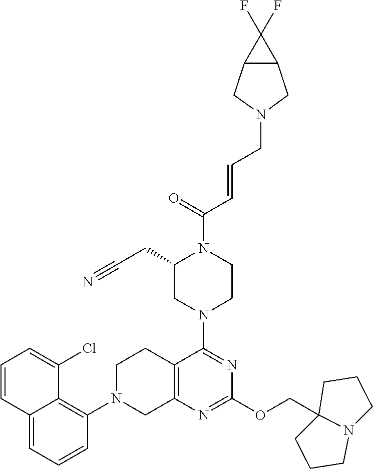 [SMILES: N#CC[C@H]1CN(c2nc(OCC34CCCN3CCC4)nc3c2CCN(c2cccc4cccc(Cl)c24)C3)CCN1C(=O)/C=C/CN1CC2C(C1)C2(F)F] | | 193 | | ++++ | | 3.13 | 97.33 |
| 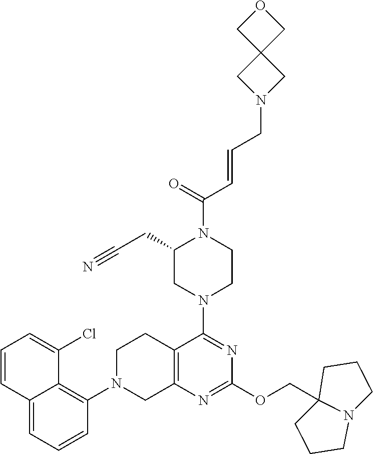 [SMILES: N#CC[C@H]1CN(c2nc(OCC34CCCN3CCC4)nc3c2CCN(c2cccc4cccc(Cl)c24)C3)CCN1C(=O)/C=C/CN1CC2(COC2)C1] | | 194 | | ++++ | | | |
| 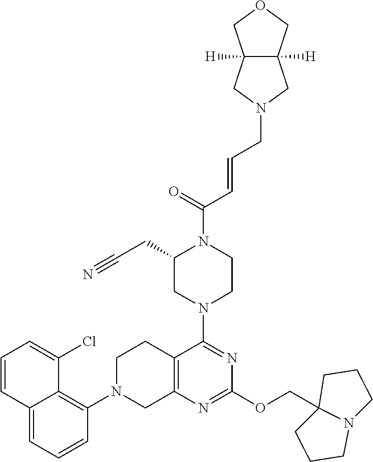 [SMILES: N#CC[C@H]1CN(c2nc(OCC34CCCN3CCC4)nc3c2CCN(c2cccc4cccc(Cl)c24)C3)CCN1C(=O)/C=C/CN1C[C@H]2COC[C@H]2C1] | | 195 | | +++ | | 2.44 | 148.84 |
| 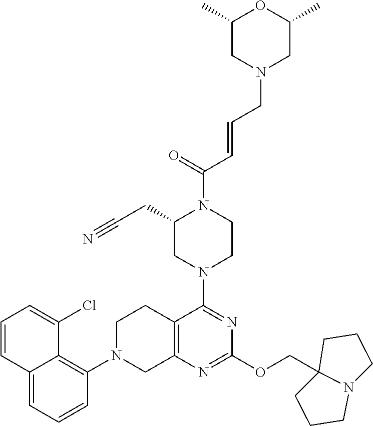 [SMILES: C[C@@H]1CN(C/C=C/C(=O)N2CCN(c3nc(OCC45CCCN4CCC5)nc4c3CCN(c3cccc5cccc(Cl)c35)C4)C[C@@H]2CC#N)C[C@H](C)O1] | | 196 | | +++ | | | |
| 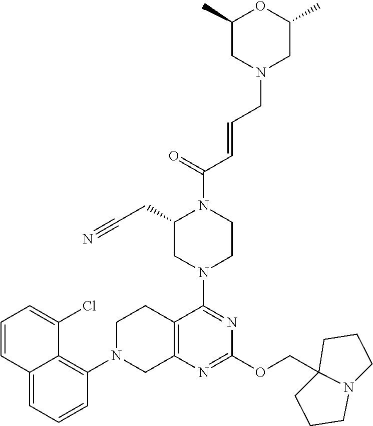 [SMILES: C[C@@H]1CN(C/C=C/C(=O)N2CCN(c3nc(OCC45CCCN4CCC5)nc4c3CCN(c3cccc5cccc(Cl)c35)C4)C[C@@H]2CC#N)C[C@@H](C)O1] | | 197 | | +++ | | | |
| 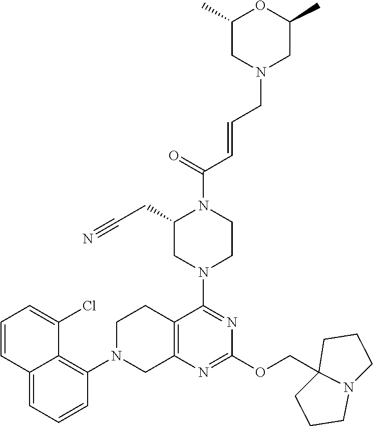 [SMILES: C[C@H]1CN(C/C=C/C(=O)N2CCN(c3nc(OCC45CCCN4CCC5)nc4c3CCN(c3cccc5cccc(Cl)c35)C4)C[C@@H]2CC#N)C[C@H](C)O1] | | 198 | | ++++ | | | |
| 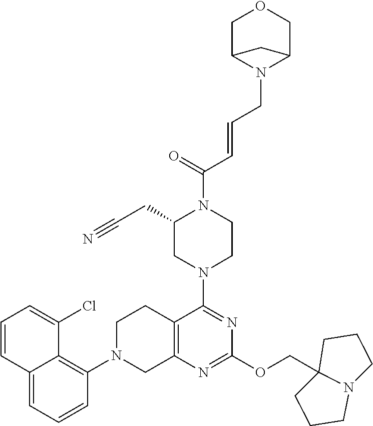 [SMILES: N#CC[C@H]1CN(c2nc(OCC34CCCN3CCC4)nc3c2CCN(c2cccc4cccc(Cl)c24)C3)CCN1C(=O)/C=C/CN1C2COCC1C2] | | 199 | | ++++ | | | |
| 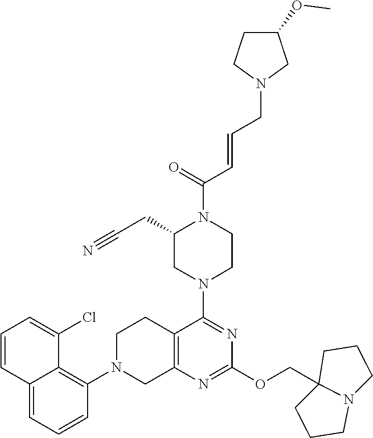 [SMILES: CO[C@H]1CCN(C/C=C/C(=O)N2CCN(c3nc(OCC45CCCN4CCC5)nc4c3CCN(c3cccc5cccc(Cl)c35)C4)C[C@@H]2CC#N)C1] | | 200 | | ++++ | | 2.42 | 137.88 |
| 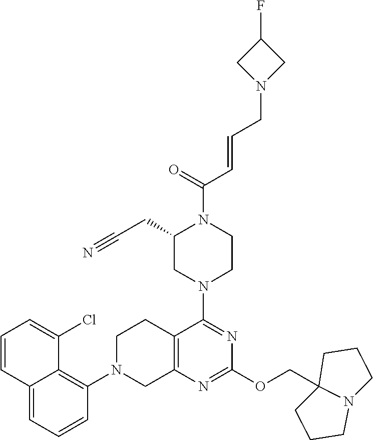 [SMILES: N#CC[C@H]1CN(c2nc(OCC34CCCN3CCC4)nc3c2CCN(c2cccc4cccc(Cl)c24)C3)CCN1C(=O)/C=C/CN1CC(F)C1] | | 201 | | +++ | | 2.76 | 119.25 |
| 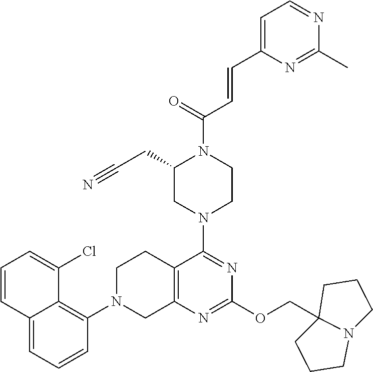 [SMILES: Cc1nccc(/C=C/C(=O)N2CCN(c3nc(OCC45CCCN4CCC5)nc4c3CCN(c3cccc5cccc(Cl)c35)C4)C[C@@H]2CC#N)n1] | | 202 | | ++++ | ++++ | 3.38 | 90.17 |
| 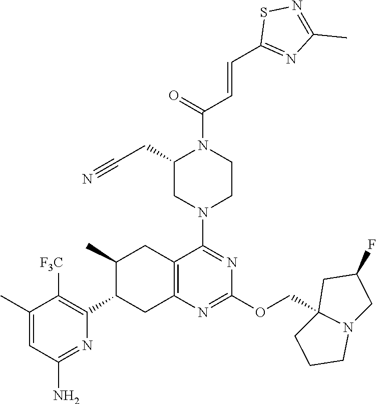 [SMILES: Cc1nsc(/C=C/C(=O)N2CCN(c3nc(OC[C@@]45CCCN4C[C@H](F)C5)nc4c3C[C@H](C)[C@@H](c3nc(N)cc(C)c3C(F)(F)F)C4)C[C@@H]2CC#N)n1] | | 203 | | +++ | | 4.59 | 0.01 |
| 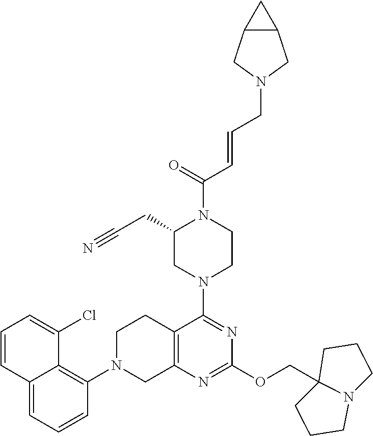 [SMILES: N#CC[C@H]1CN(c2nc(OCC34CCCN3CCC4)nc3c2CCN(c2cccc4cccc(Cl)c24)C3)CCN1C(=O)/C=C/CN1CC2CC2C1] | | 204 | | ++++ | | | |
| 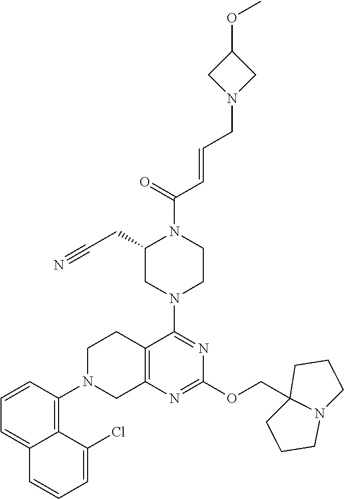 [SMILES: COC1CN(C/C=C/C(=O)N2CCN(c3nc(OCC45CCCN4CCC5)nc4c3CCN(c3cccc5cccc(Cl)c35)C4)C[C@@H]2CC#N)C1] | | 205 | | ++++ | | | |
| 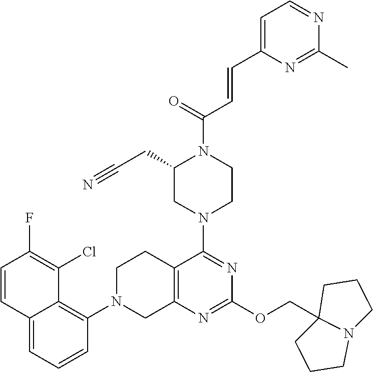 [SMILES: Cc1nccc(/C=C/C(=O)N2CCN(c3nc(OCC45CCCN4CCC5)nc4c3CCN(c3cccc5ccc(F)c(Cl)c35)C4)C[C@@H]2CC#N)n1] | | 206 | | ++ | | | |
| 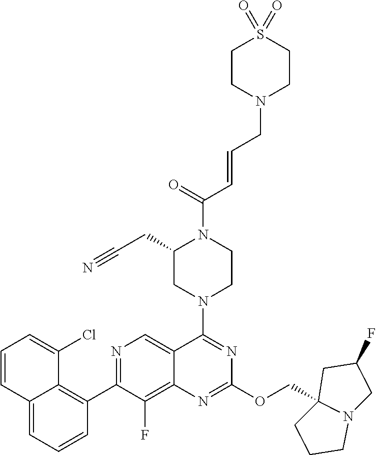 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCS(=O)(=O)CC1] | | 207 | | ++ | | 2.40 | 29.93 |
| 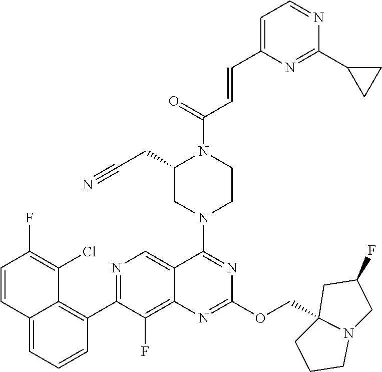 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/c1ccnc(C2CC2)n1] | | 208 | | ++++ | ++++ | 4.20 | 0.01 |
| 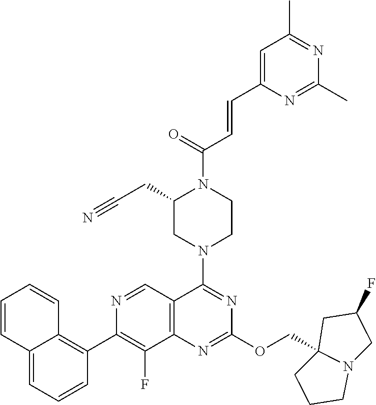 [SMILES: Cc1cc(/C=C/C(=O)N2CCN(c3nc(OC[C@@]45CCCN4C[C@H](F)C5)nc4c(F)c(-c5cccc6ccccc56)ncc34)C[C@@H]2CC#N)nc(C)n1] | | 209 | | ++ | | | |
| 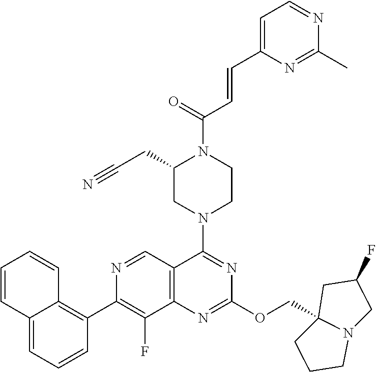 [SMILES: Cc1nccc(/C=C/C(=O)N2CCN(c3nc(OC[C@@]45CCCN4C[C@H](F)C5)nc4c(F)c(-c5cccc6ccccc56)ncc34)C[C@@H]2CC#N)n1] | | 210 | | ++ | | | |
| 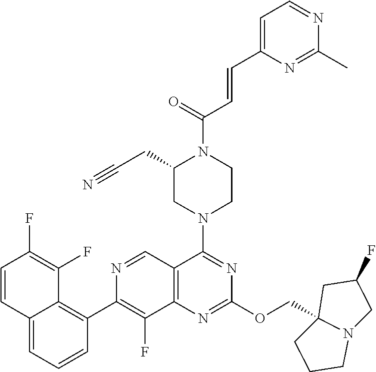 [SMILES: Cc1nccc(/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)n1] | | 211 | | ++++ | ++++ | 3.41 | 11.96 |
| 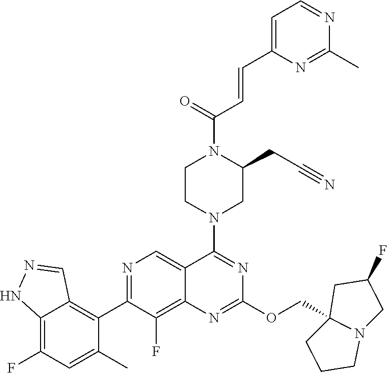 [SMILES: Cc1nccc(/C=C/C(=O)N2CCN(c3nc(OC[C@@]45CCCN4C[C@H](F)C5)nc4c(F)c(-c5c(C)cc(F)c6[nH]ncc56)ncc34)C[C@@H]2CC#N)n1] | | 212 | | ++ | | | |
| 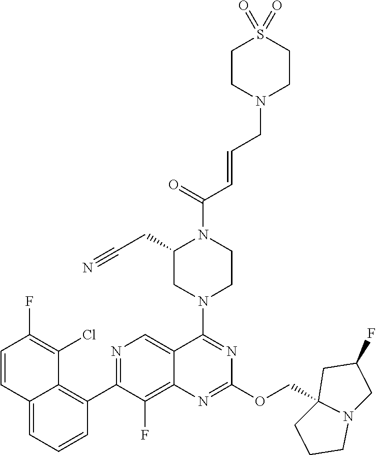 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCS(=O)(=O)CC1] | | 213 | | ++ | | | |
| 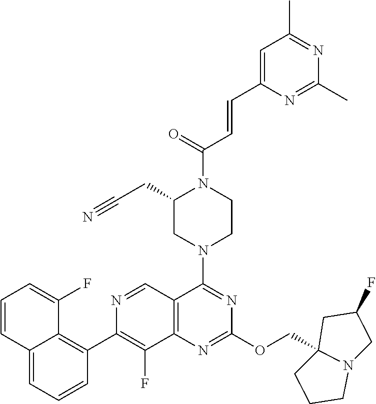 [SMILES: Cc1cc(/C=C/C(=O)N2CCN(c3nc(OC[C@@]45CCCN4C[C@H](F)C5)nc4c(F)c(-c5cccc6cccc(F)c56)ncc34)C[C@@H]2CC#N)nc(C)n1] | | 214 | | +++ | ++++ | 4.02 | 6.14 |
| 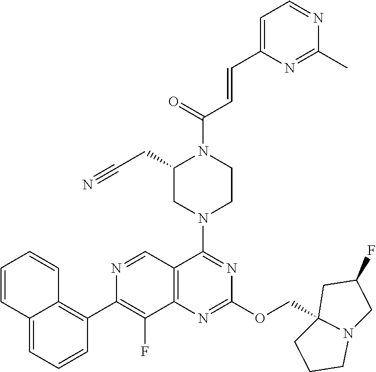 [SMILES: Cc1nccc(/C=C/C(=O)N2CCN(c3nc(OC[C@@]45CCCN4C[C@H](F)C5)nc4c(F)c(-c5cccc6ccccc56)ncc34)C[C@@H]2CC#N)n1] | | 215 | | ++++ | ++++ | 2.16 | 16.54 |
| 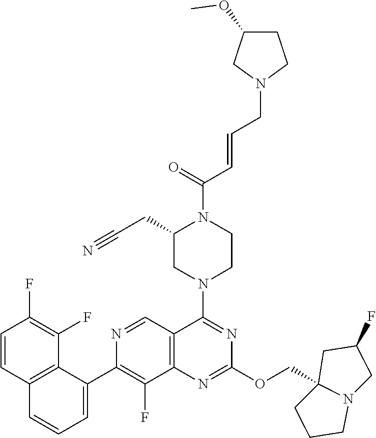 [SMILES: CO[C@@H]1CCN(C/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)C1] | | 216 | | ++++ | ++++ | | |
| 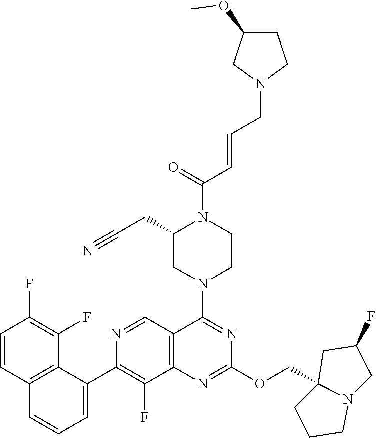 [SMILES: CO[C@H]1CCN(C/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)C1] | | 217 | | ++++ | ++++ | 2.01 | 99.94 |
| 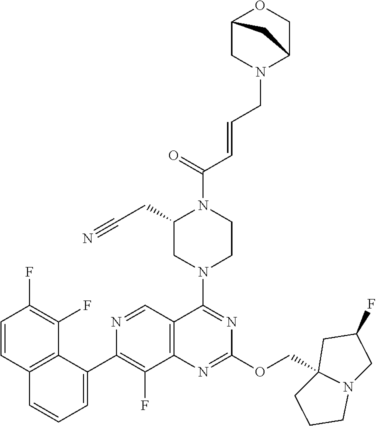 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1C[C@H]2C[C@@H]1CO2] | | 218 | | ++++ | ++++ | 2.54 | 56.42 |
| 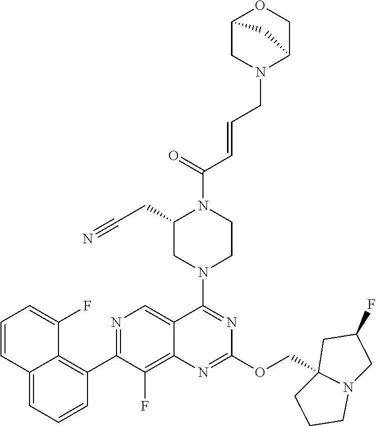 [SMILES: N#CC[C@H]1CN(c2nc(OC[C@@]34CCCN3C[C@H](F)C4)nc3c(F)c(-c4cccc5cccc(F)c45)ncc23)CCN1C(=O)/C=C/CN1C[C@@H]2C[C@H]1CO2] | | 219 | | ++++ | ++++ | 2.50 | 79.41 |
| 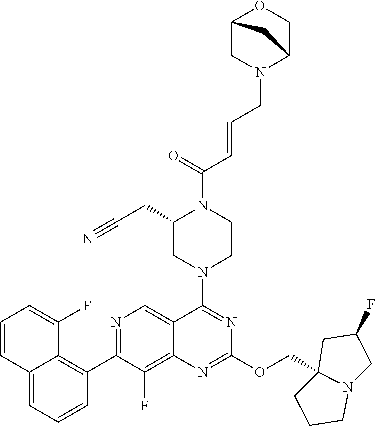 [SMILES: N#CC[C@H]1CN(c2nc(OC[C@@]34CCCN3C[C@H](F)C4)nc3c(F)c(-c4cccc5cccc(F)c45)ncc23)CCN1C(=O)/C=C/CN1C[C@H]2C[C@@H]1CO2] | | 220 | | ++++ | ++++ | 2.43 | 98.45 |
| 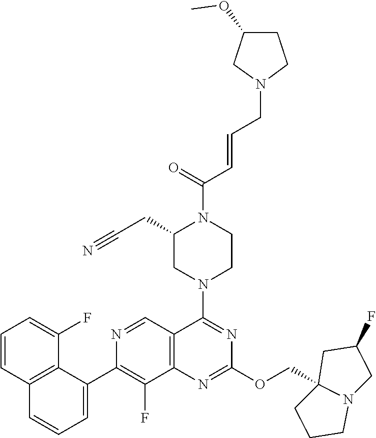 [SMILES: CO[C@@H]1CCN(C/C=C/C(=O)N2CCN(c3nc(OC[C@@]45CCCN4C[C@H](F)C5)nc4c(F)c(-c5cccc6cccc(F)c56)ncc34)C[C@@H]2CC#N)C1] | | 221 | | ++++ | | | |
| 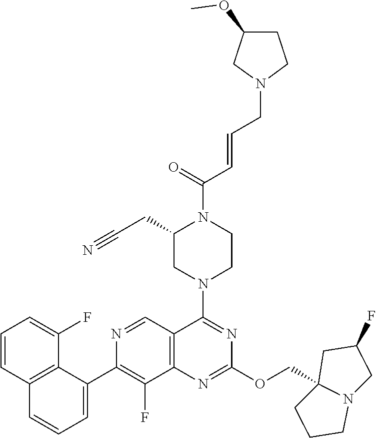 [SMILES: CO[C@H]1CCN(C/C=C/C(=O)N2CCN(c3nc(OC[C@@]45CCCN4C[C@H](F)C5)nc4c(F)c(-c5cccc6cccc(F)c56)ncc34)C[C@@H]2CC#N)C1] | | 222 | | ++++ | ++++ | 2.76 | 91.60 |
| 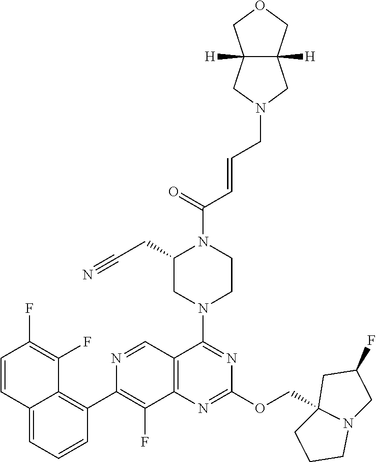 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1C[C@H]2COC[C@H]2C1] | | 223 | | ++++ | ++++ | 3.00 | 118.11 |
| 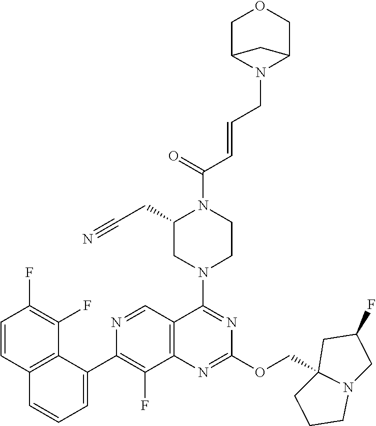 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1C2COCC1C2] | | 224 | | ++++ | ++++ | 2.96 | 84.58 |
| 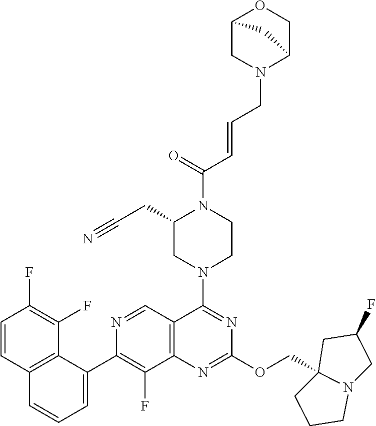 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1C[C@@H]2C[C@H]1CO2] | | 225 | | ++++ | | 1.87 | 134.69 |
| 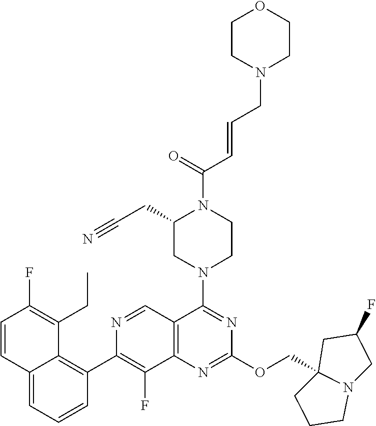 [SMILES: CCc1c(F)ccc2cccc(-c3ncc4c(N5CCN(C(=O)/C=C/CN6CCOCC6)[C@@H](CC#N)C5)nc(OC[C@@]56CCCN5C[C@H](F)C6)nc4c3F)c12] | | 226 | | +++ | ++++ | 2.63 | 103.11 |
| 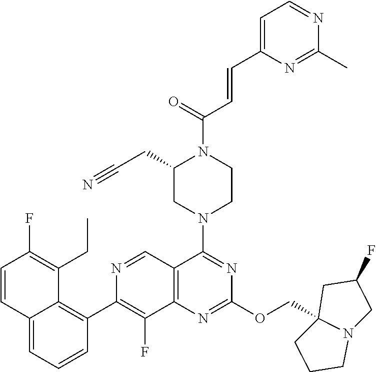 [SMILES: CCc1c(F)ccc2cccc(-c3ncc4c(N5CCN(C(=O)/C=C/c6ccnc(C)n6)[C@@H](CC#N)C5)nc(OC[C@@]56CCCN5C[C@H](F)C6)nc4c3F)c12] | | 227 | | ++++ | ++++ | 3.87 | 5.49 |
| 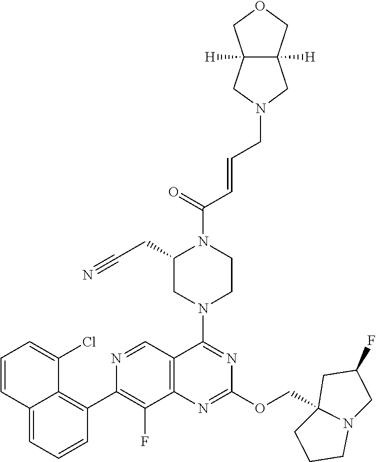 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@H]2COC[C@H]2C1] | | 228 | | ++++ | ++++ | 3.08 | 94.77 |
| 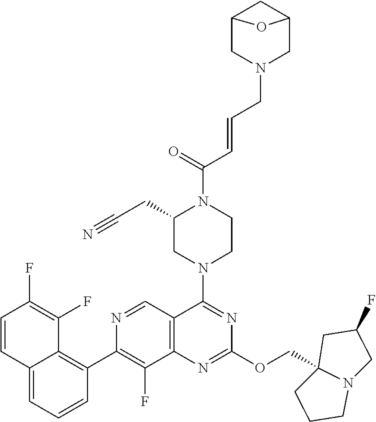 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CC2CC(C1)O2] | | 229 | | ++++ | ++++ | 1.63 | 69.89 |
| 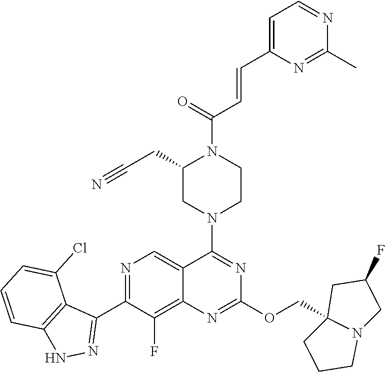 [SMILES: Cc1nccc(/C=C/C(=O)N2CCN(c3nc(OC[C@@]45CCCN4C[C@H](F)C5)nc4c(F)c(-c5n[nH]c6cccc(Cl)c56)ncc34)C[C@@H]2CC#N)n1] | | 230 | | + | | | |
| 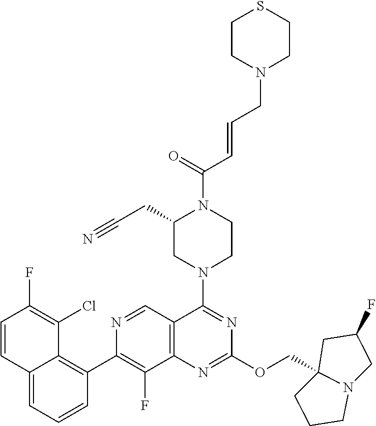 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCSCC1] | | 231 | | ++++ | | 4.07 | 9.38 |
| 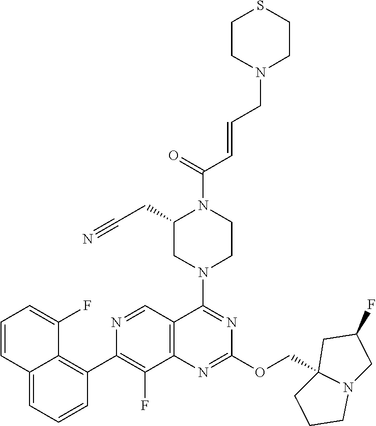 [SMILES: N#CC[C@H]1CN(c2nc(OC[C@@]34CCCN3C[C@H](F)C4)nc3c(F)c(-c4cccc5cccc(F)c45)ncc23)CCN1C(=O)/C=C/CN1CCSCC1] | | 232 | | ++++ | | 3.68 | 34.82 |
| 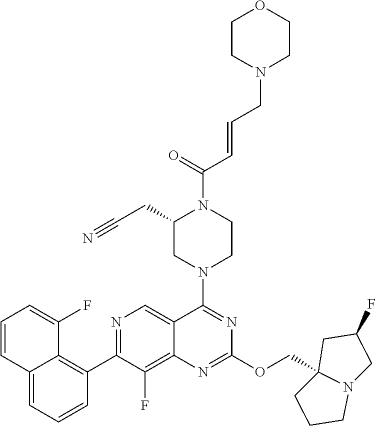 [SMILES: N#CC[C@H]1CN(c2nc(OC[C@@]34CCCN3C[C@H](F)C4)nc3c(F)c(-c4cccc5cccc(F)c45)ncc23)CCN1C(=O)/C=C/CN1CCOCC1] | | 233 | | +++ | | 2.91 | 102.33 |
| 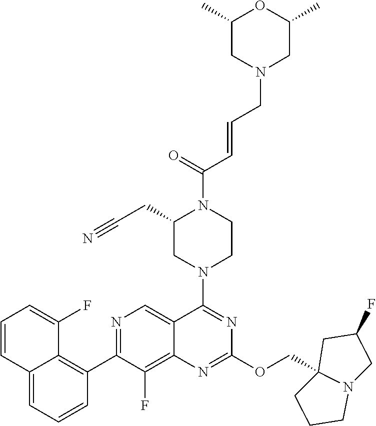 [SMILES: C[C@@H]1CN(C/C=C/C(=O)N2CCN(c3nc(OC[C@@]45CCCN4C[C@H](F)C5)nc4c(F)c(-c5cccc6cccc(F)c56)ncc34)C[C@@H]2CC#N)C[C@H](C)O1] | | 234 | | +++ | | | |
| 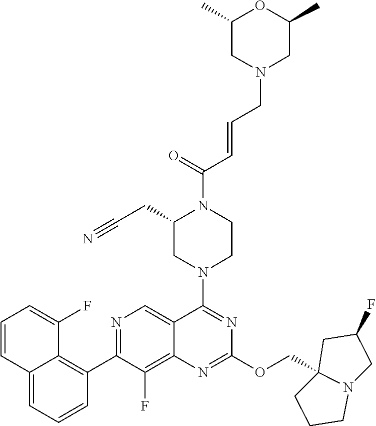 [SMILES: C[C@H]1CN(C/C=C/C(=O)N2CCN(c3nc(OC[C@@]45CCCN4C[C@H](F)C5)nc4c(F)c(-c5cccc6cccc(F)c56)ncc34)C[C@@H]2CC#N)C[C@H](C)O1] | | 235 | | +++ | | 3.75 | 72.54 |
| 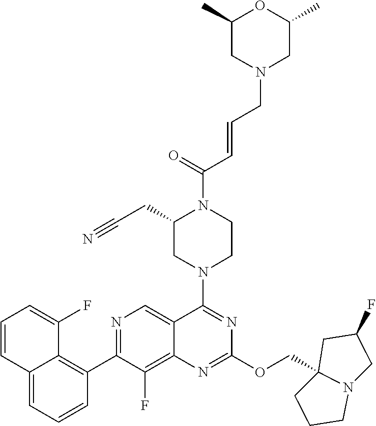 [SMILES: C[C@@H]1CN(C/C=C/C(=O)N2CCN(c3nc(OC[C@@]45CCCN4C[C@H](F)C5)nc4c(F)c(-c5cccc6cccc(F)c56)ncc34)C[C@@H]2CC#N)C[C@@H](C)O1] | | 236 | | ++ | | 3.59 | 88.02 |
| 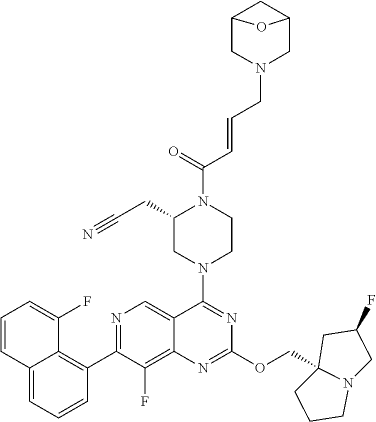 [SMILES: N#CC[C@H]1CN(c2nc(OC[C@@]34CCCN3C[C@H](F)C4)nc3c(F)c(-c4cccc5cccc(F)c45)ncc23)CCN1C(=O)/C=C/CN1CC2CC(C1)O2] | | 237 | | ++++ | | 2.87 | 67.04 |
| 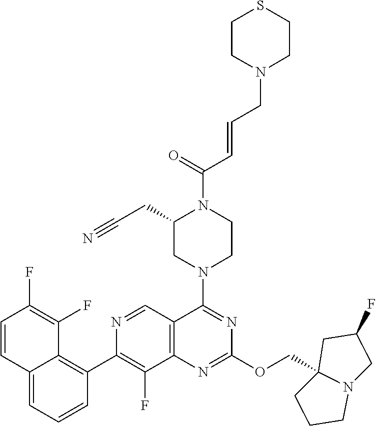 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCSCC1] | | 238 | | ++++ | ++++ | 3.48 | 21.31 |
| 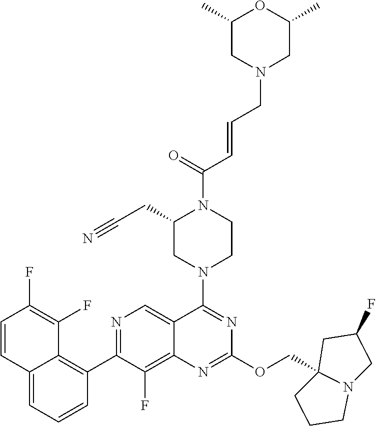 [SMILES: C[C@@H]1CN(C/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)C[C@H](C)O1] | | 239 | | +++ | | 2.53 | 92.61 |
| 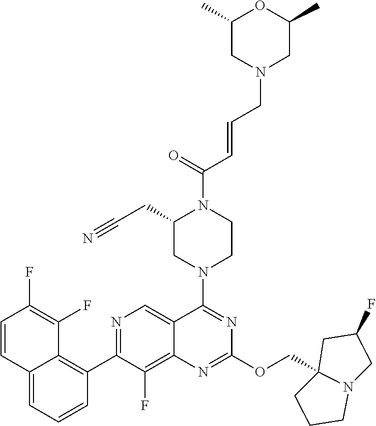 [SMILES: C[C@H]1CN(C/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)C[C@H](C)O1] | | 240 | | +++ | | 3.42 | 49.46 |
| 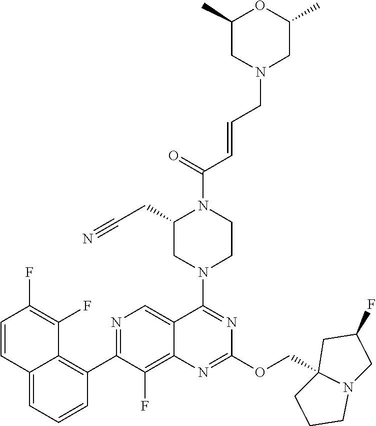 [SMILES: C[C@@H]1CN(C/C=C/C(=O)N2CCN(c3nc(OC[C@@]45CCCN4C[C@H](F)C5)nc4c(F)c(-c5cccc6ccc(F)c(F)c56)ncc34)C[C@@H]2CC#N)C[C@@H](C)O1] | | 241 | | +++ | | 2.94 | 60.07 |
| 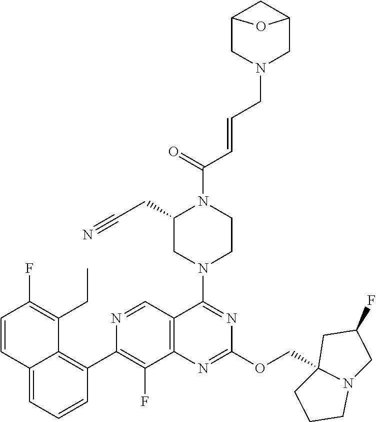 [SMILES: CCc1c(F)ccc2cccc(-c3ncc4c(N5CCN(C(=O)/C=C/CN6CC7CC(C6)O7)[C@@H](CC#N)C5)nc(OC[C@@]56CCCN5C[C@H](F)C6)nc4c3F)c12] | | 242 | | ++++ | | 3.38 | 93.35 |
| 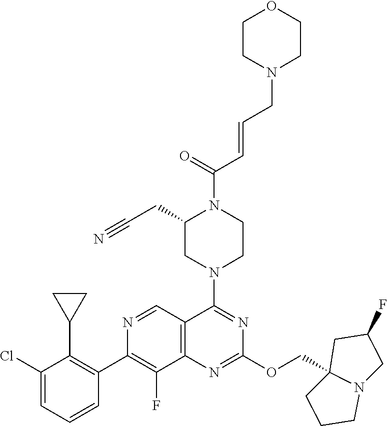 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CCOCC1] | | 243 | | ++++ | ++++ | 3.14 | 105.54 |
| 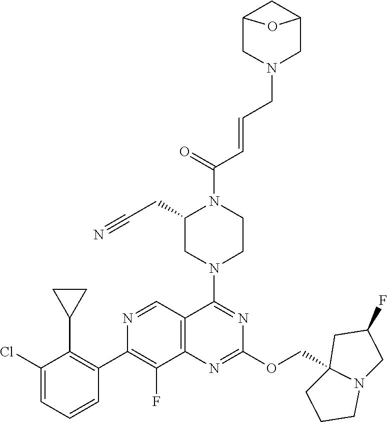 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CC2CC(C1)O2] | | 244 | | ++++ | +++ | 3.13 | 121.25 |
| 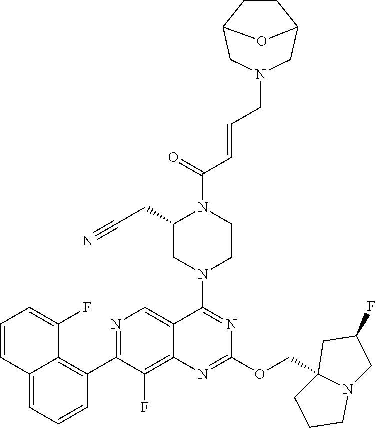 [SMILES: N#CC[C@H]1CN(c2nc(OC[C@@]34CCCN3C[C@H](F)C4)nc3c(F)c(-c4cccc5cccc(F)c45)ncc23)CCN1C(=O)/C=C/CN1CC2CCC(C1)O2] | | 245 | | +++ | | 2.91 | 74.37 |
| 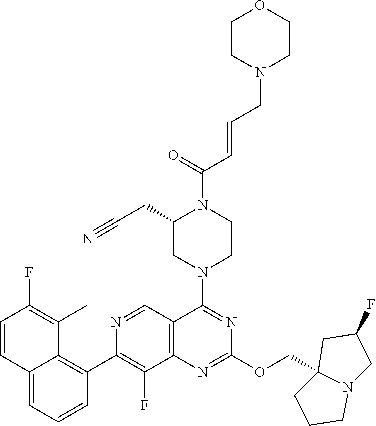 [SMILES: Cc1c(F)ccc2cccc(-c3ncc4c(N5CCN(C(=O)/C=C/CN6CCOCC6)[C@@H](CC#N)C5)nc(OC[C@@]56CCCN5C[C@H](F)C6)nc4c3F)c12] | | 246 | | +++ | | 2.96 | 69.55 |
| 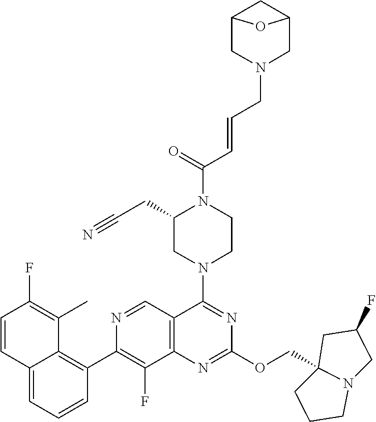 [SMILES: Cc1c(F)ccc2cccc(-c3ncc4c(N5CCN(C(=O)/C=C/CN6CC7CC(C6)O7)[C@@H](CC#N)C5)nc(OC[C@@]56CCCN5C[C@H](F)C6)nc4c3F)c12] | | 247 | | ++++ | | 3.02 | 88.61 |
| 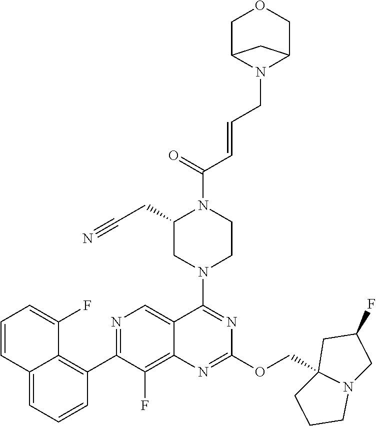 [SMILES: N#CC[C@H]1CN(c2nc(OC[C@@]34CCCN3C[C@H](F)C4)nc3c(F)c(-c4cccc5cccc(F)c45)ncc23)CCN1C(=O)/C=C/CN1C2COCC1C2] | | 248 | | +++ | | | |
| 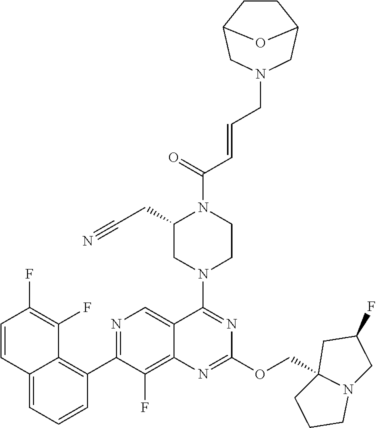 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CC2CCC(C1)O2] | | 249 | | ++++ | | 3.38 | 71.10 |
| 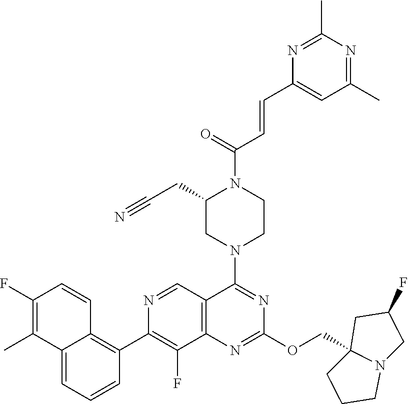 [SMILES: Cc1cc(/C=C/C(=O)N2CCN(c3nc(OC[C@@]45CCCN4C[C@H](F)C5)nc4c(F)c(-c5cccc6c(C)c(F)ccc56)ncc34)C[C@@H]2CC#N)nc(C)n1] | | 250 | | ++ | | | |
| 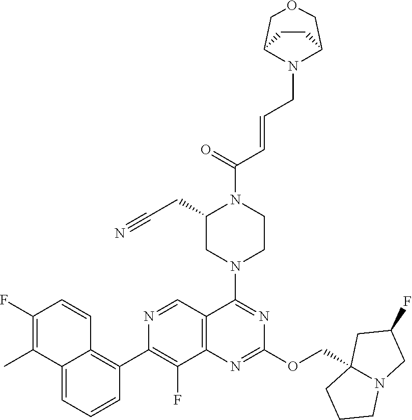 [SMILES: Cc1c(F)ccc2c(-c3ncc4c(N5CCN(C(=O)/C=C/CN6[C@@H]7CC[C@H]6COC7)[C@@H](CC#N)C5)nc(OC[C@@]56CCCN5C[C@H](F)C6)nc4c3F)cccc12] | | 251 | | ++ | | | |
| 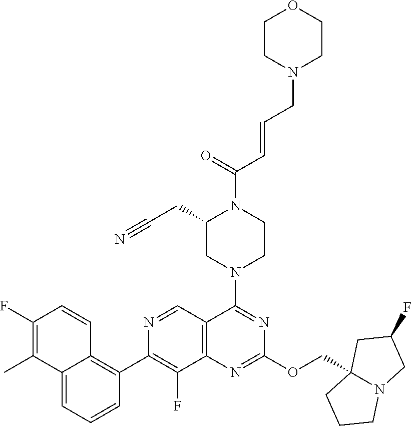 [SMILES: Cc1c(F)ccc2c(-c3ncc4c(N5CCN(C(=O)/C=C/CN6CCOCC6)[C@@H](CC#N)C5)nc(OC[C@@]56CCCN5C[C@H](F)C6)nc4c3F)cccc12] | | 252 | | ++ | | | |
| 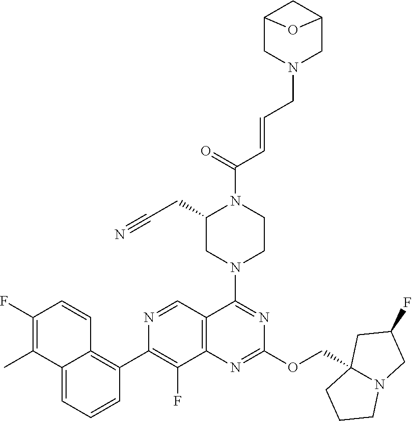 [SMILES: Cc1c(F)ccc2c(-c3ncc4c(N5CCN(C(=O)/C=C/CN6CC7CC(C6)O7)[C@@H](CC#N)C5)nc(OC[C@@]56CCCN5C[C@H](F)C6)nc4c3F)cccc12] | | 253 | | + | | | |
| 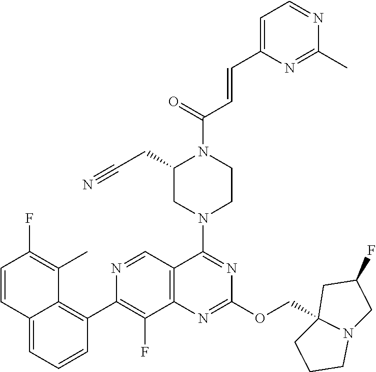 [SMILES: Cc1nccc(/C=C/C(=O)N2CCN(c3nc(OC[C@@]45CCCN4C[C@H](F)C5)nc4c(F)c(-c5cccc6ccc(F)c(C)c56)ncc34)C[C@@H]2CC#N)n1] | | 254 | | ++++ | | 3.79 | 4.99 |
| 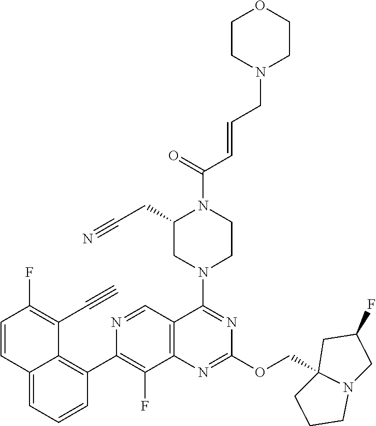 [SMILES: C#Cc1c(F)ccc2cccc(-c3ncc4c(N5CCN(C(=O)/C=C/CN6CCOCC6)[C@@H](CC#N)C5)nc(OC[C@@]56CCCN5C[C@H](F)C6)nc4c3F)c12] | | 255 | | ++++ | ++++ | 2.81 | 118.36 |
| 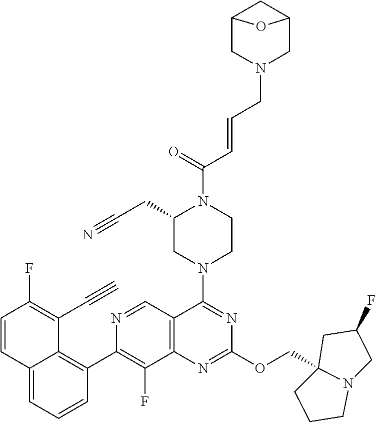 [SMILES: C#Cc1c(F)ccc2cccc(-c3ncc4c(N5CCN(C(=O)/C=C/CN6CC7CC(C6)O7)[C@@H](CC#N)C5)nc(OC[C@@]56CCCN5C[C@H](F)C6)nc4c3F)c12] | | 256 | | ++++ | | 2.71 | 101.27 |
| 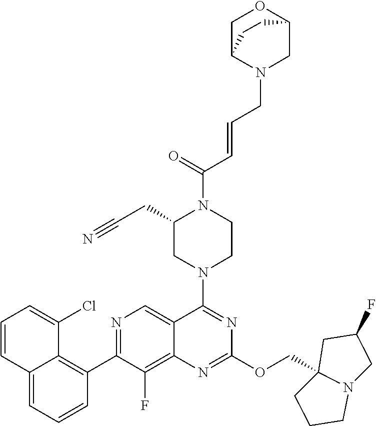 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@H]2CC[C@@H]1CO2] | | 258 | | ++++ | | 2.81 | 168.80 |
| 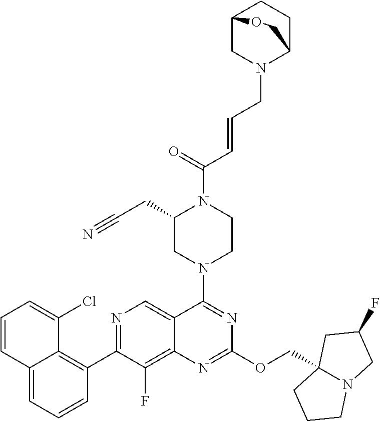 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@@H]2CC[C@H]1CO2] | | 259 | | ++++ | | 2.92 | 147.27 |
| 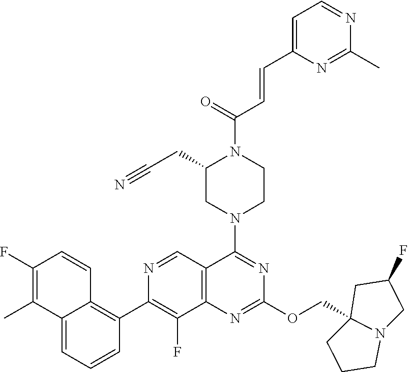 [SMILES: Cc1nccc(/C=C/C(=O)N2CCN(c3nc(OC[C@@]45CCCN4C[C@H](F)C5)nc4c(F)c(-c5cccc6c(C)c(F)ccc56)ncc34)C[C@@H]2CC#N)n1] | | 260 | | + | | | |
| 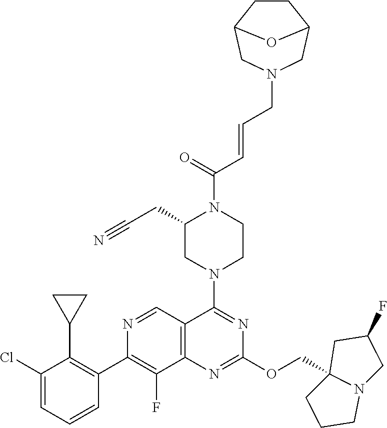 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CC2CCC(C1)O2] | | 261 | | +++ | | 3.14 | 56.70 |
| 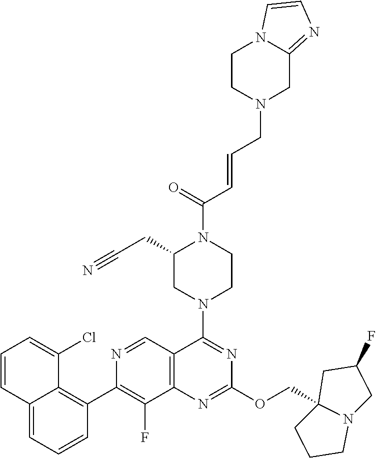 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCn2ccnc2C1] | | 262 | | +++ | | | |
| 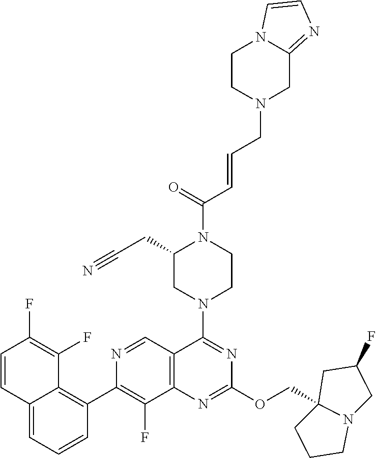 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCn2ccnc2C1] | | 263 | | +++ | | | |
| 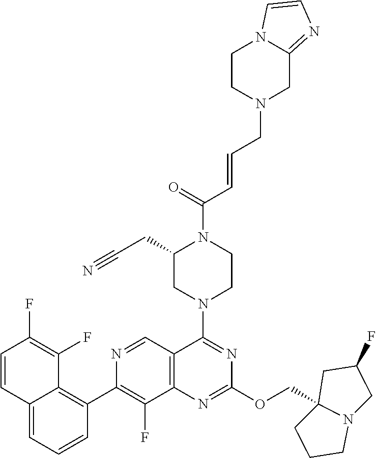 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCn2ccnc2C1] | | 264 | | ++ | | | |
| 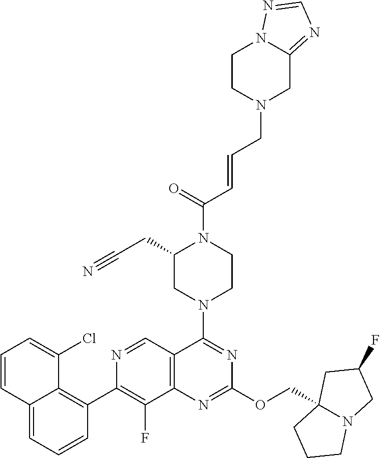 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCn2ncnc2C1] | | 265 | | ++ | | | |
| 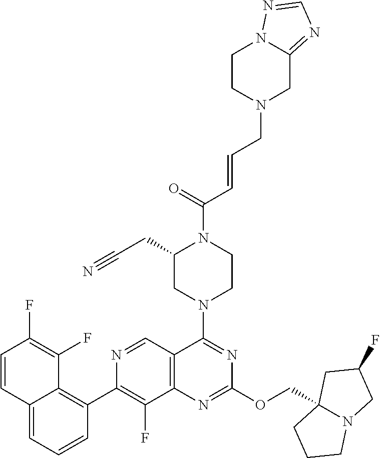 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCn2ncnc2C1] | | 266 | | ++ | | | |
| 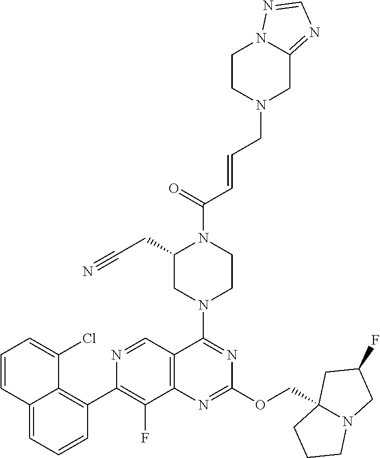 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCn2ncnc2C1] | | 267 | | +++ | | | |
| 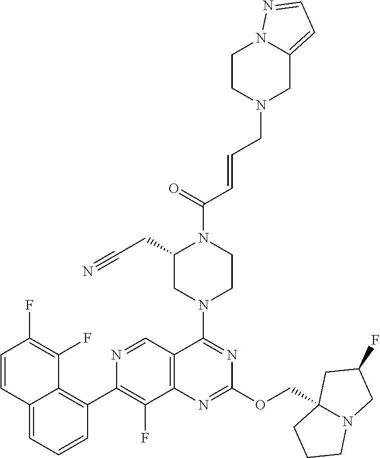 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCn2nccc2C1] | | 268 | | +++ | | | |
| 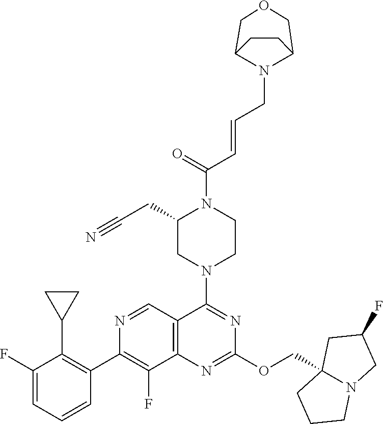 [SMILES: N#CC[C@H]1CN(c2nc(OC[C@@]34CCCN3C[C@H](F)C4)nc3c(F)c(-c4cccc(F)c4C4CC4)ncc23)CCN1C(=O)/C=C/CN1C2CCC1COC2] | | 269 | | ++++ | | | |
| 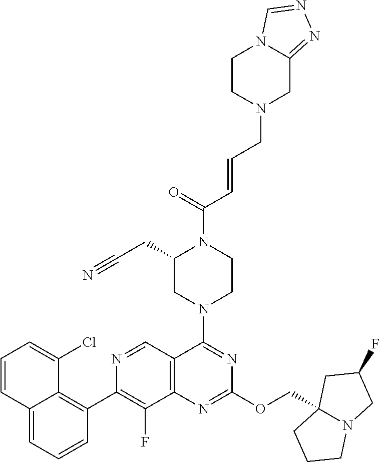 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCn2cnnc2C1] | | 270 | | ++ | | | |
| 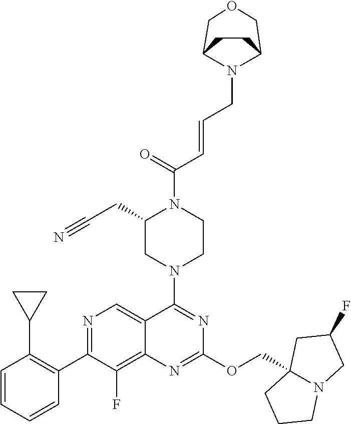 [SMILES: N#CC[C@H]1CN(c2nc(OC[C@@]34CCCN3C[C@H](F)C4)nc3c(F)c(-c4ccccc4C4CC4)ncc23)CCN1C(=O)/C=C/CN1[C@@H]2CC[C@H]1COC2] | | 271 | | +++ | | | |
| 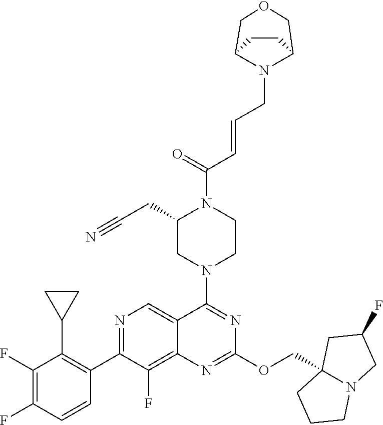 [SMILES: N#CC[C@H]1CN(c2nc(OC[C@@]34CCCN3C[C@H](F)C4)nc3c(F)c(-c4ccc(F)c(F)c4C4CC4)ncc23)CCN1C(=O)/C=C/CN1[C@@H]2CC[C@H]1COC2] | | 272 | | +++ | | | |
| 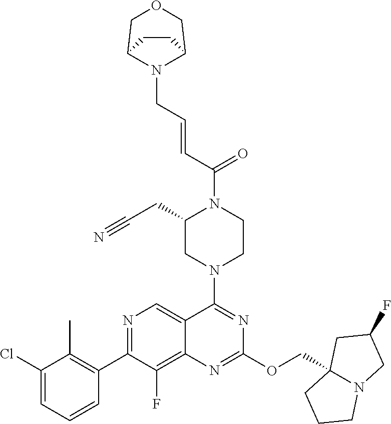 [SMILES: Cc1c(Cl)cccc1-c1ncc2c(N3CCN(C(=O)/C=C/CN4[C@@H]5CC[C@H]4COC5)[C@@H](CC#N)C3)nc(OC[C@@]34CCCN3C[C@H](F)C4)nc2c1F] | | 273 | | ++++ | | 3.10 | 124.04 |
| 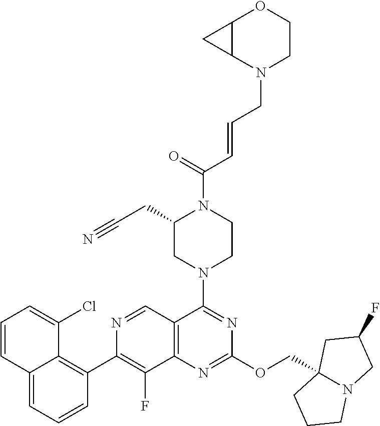 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCOC2CC21] | | 274 | | +++ | | 3.14 | 76.22 |
| 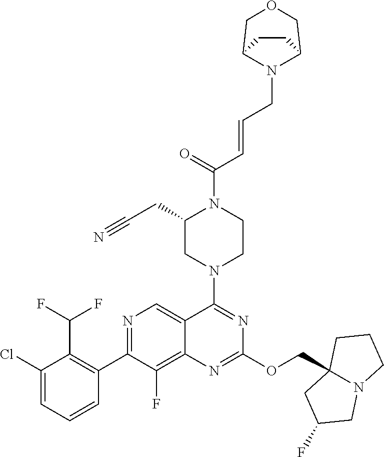 [SMILES: N#CC[C@H]1CN(c2nc(OC[C@@]34CCCN3C[C@H](F)C4)nc3c(F)c(-c4cccc(Cl)c4C(F)F)ncc23)CCN1C(=O)/C=C/CN1[C@@H]2CC[C@H]1COC2] | | 275 | | ++++ | | 2.73 | 105.61 |
| 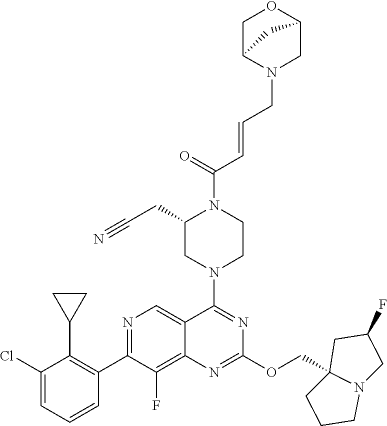 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1C[C@H]2C[C@@H]1CO2] | | 276 | | ++++ | | 3.00 | 107.84 |
| 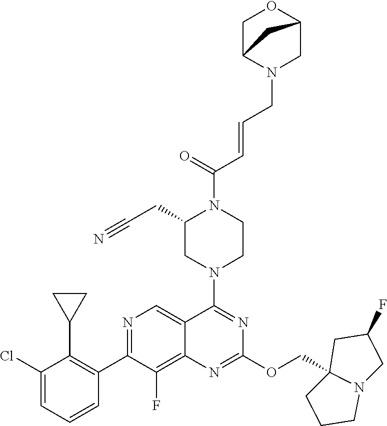 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1C[C@@H]2C[C@H]1CO2] | | 277 | | ++++ | ++++ | 2.85 | 91.01 |
| 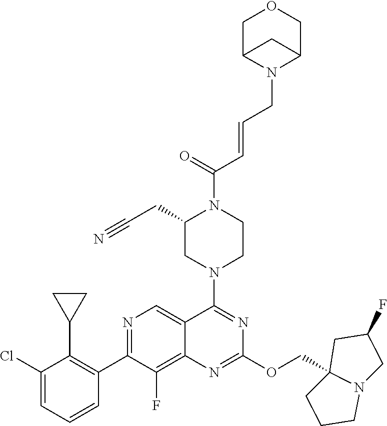 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1C2COCC1C2] | | 278 | | ++++ | | 3.30 | 66.60 |
| 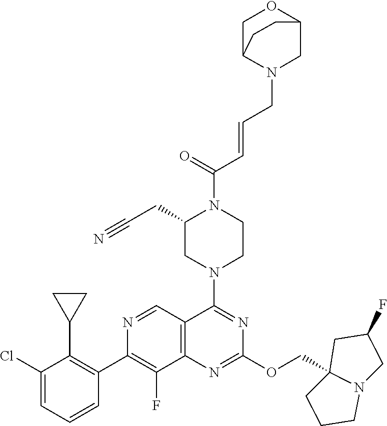 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CC2CCC1CO2] | | 279 | | ++++ | | 3.35 | 54.11 |
| 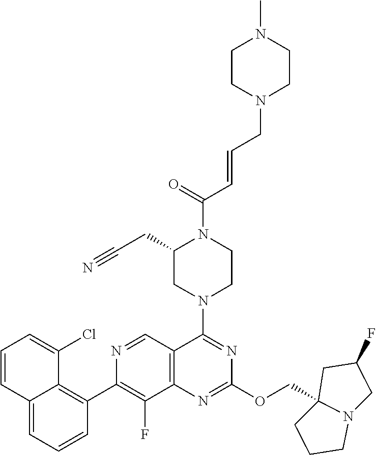 [SMILES: CN1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)CC1] | | 280 | | +++ | | | |
| 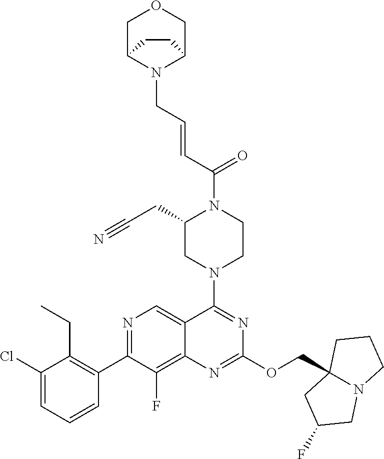 [SMILES: CCc1c(Cl)cccc1-c1ncc2c(N3CCN(C(=O)/C=C/CN4[C@@H]5CC[C@H]4COC5)[C@@H](CC#N)C3)nc(OC[C@@]34CCCN3C[C@H](F)C4)nc2c1F] | | 281 | | +++ | | | |
| 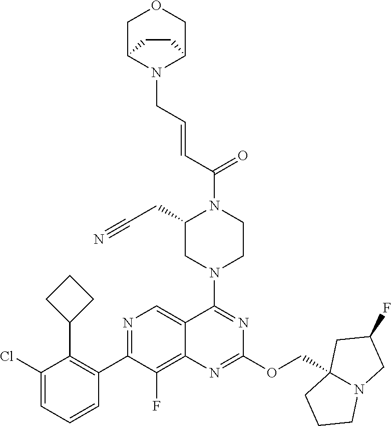 [SMILES: N#CC[C@H]1CN(c2nc(OC[C@@]34CCCN3C[C@H](F)C4)nc3c(F)c(-c4cccc(Cl)c4C4CCC4)ncc23)CCN1C(=O)/C=C/CN1[C@@H]2CC[C@H]1COC2] | | 282 | | +++ | | | |
| 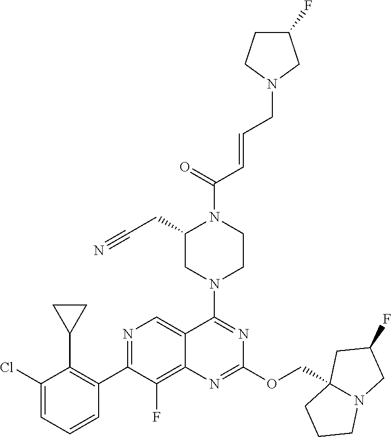 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CC[C@H](F)C1] | | 283 | | ++++ | ++++ | 3.45 | 134.57 |
| 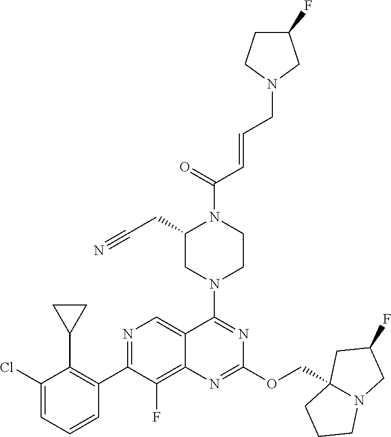 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CC[C@@H](F)C1] | | 284 | | ++++ | | 3.27 | 123.82 |
| 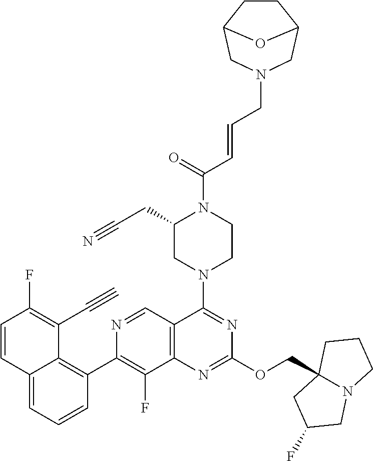 [SMILES: C#Cc1c(F)ccc2cccc(-c3ncc4c(N5CCN(C(=O)/C=C/CN6CC7CCC(C6)O7)[C@@H](CC#N)C5)nc(OC[C@@]56CCCN5C[C@H](F)C6)nc4c3F)c12] | | 285 | | ++++ | | 3.54 | 70.40 |
| 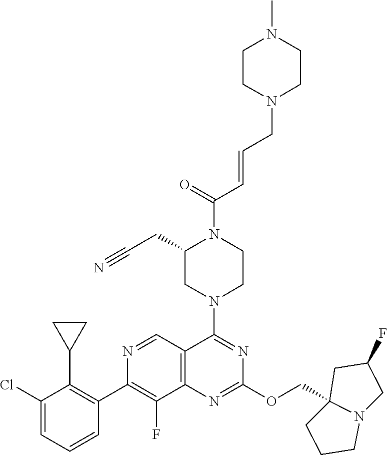 [SMILES: CN1CCN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)CC1] | | 286 | | +++ | | | |
| 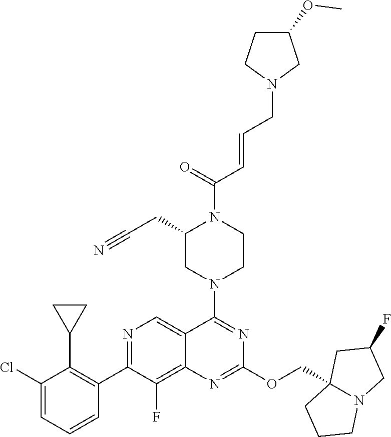 [SMILES: CO[C@H]1CCN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)C1] | | 287 | | ++++ | | 3.23 | 113.70 |
| 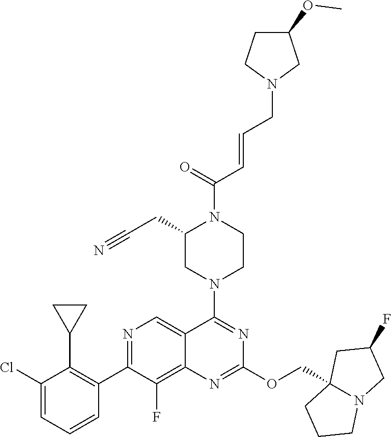 [SMILES: CO[C@@H]1CCN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)C1] | | 288 | | ++++ | | | |
| 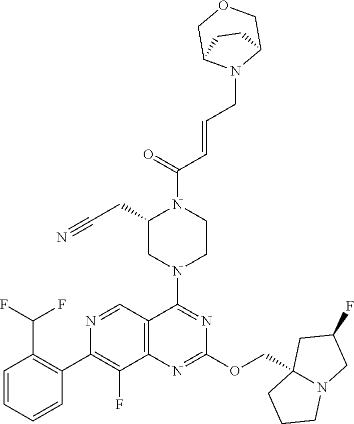 [SMILES: N#CC[C@H]1CN(c2nc(OC[C@@]34CCCN3C[C@H](F)C4)nc3c(F)c(-c4ccccc4C(F)F)ncc23)CCN1C(=O)/C=C/CN1[C@@H]2CC[C@H]1COC2] | | 289 | | +++ | | 2.16 | 139.84 |
| 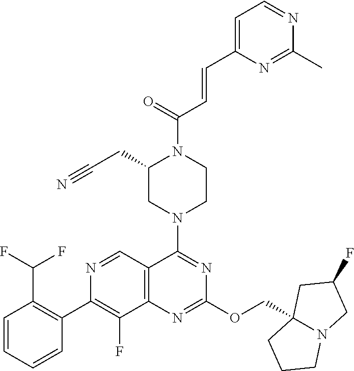 [SMILES: Cc1nccc(/C=C/C(=O)N2CCN(c3nc(OC[C@@]45CCCN4C[C@H](F)C5)nc4c(F)c(-c5ccccc5C(F)F)ncc34)C[C@@H]2CC#N)n1] | | 290 | | + | | 2.71 | 115.05 |
| 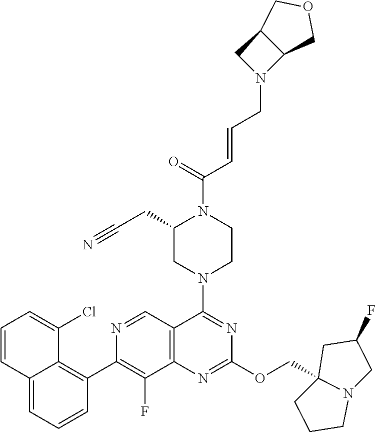 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@H]2COC[C@H]21] | | 291 | | +++ | | | |
| 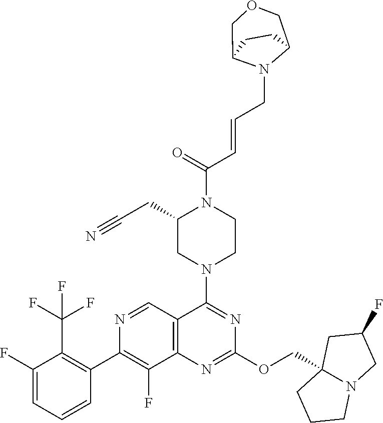 [SMILES: N#CC[C@H]1CN(c2nc(OC[C@@]34CCCN3C[C@H](F)C4)nc3c(F)c(-c4cccc(F)c4C(F)(F)F)ncc23)CCN1C(=O)/C=C/CN1[C@@H]2CC[C@H]1COC2] | | 292 | | ++++ | | 2.54 | 111.91 |
| 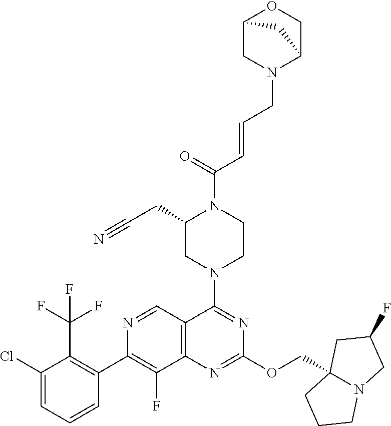 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1C[C@@H]2C[C@H]1CO2] | | 293 | | ++++ | | 2.54 | 96.67 |
| 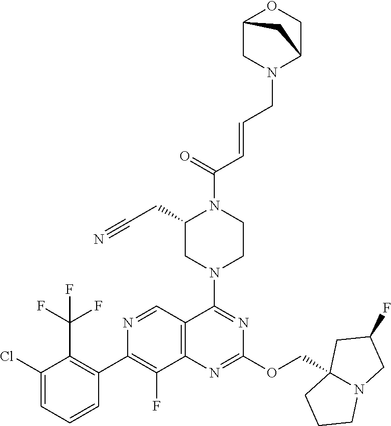 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1C[C@H]2C[C@@H]1CO2] | | 294 | | ++++ | | 2.59 | 127.42 |
| 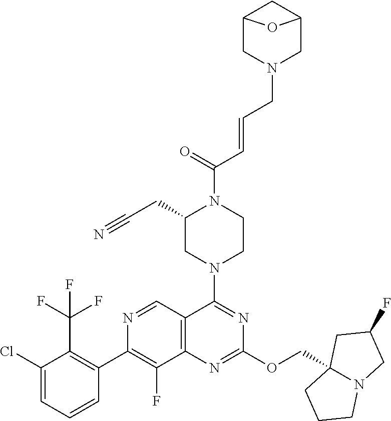 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CC2CC(C1)O2] | | 295 | | ++++ | | 2.75 | 102.36 |
| 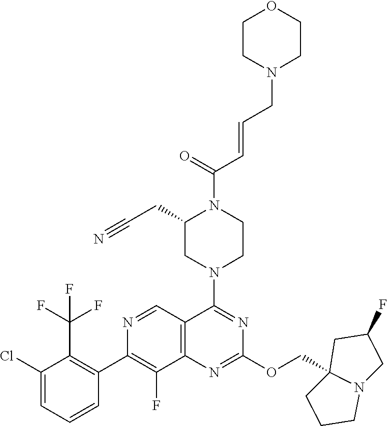 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CCOCC1] | | 296 | | ++++ | | 2.76 | 100.17 |
| 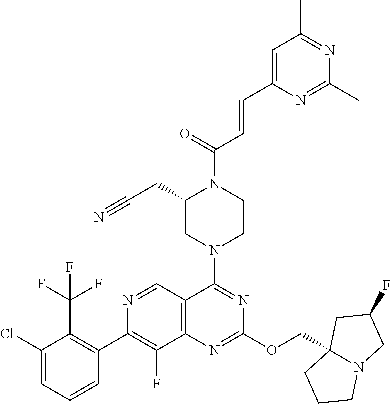 [SMILES: Cc1cc(/C=C/C(=O)N2CCN(c3nc(OC[C@@]45CCCN4C[C@H](F)C5)nc4c(F)c(-c5cccc(Cl)c5C(F)(F)F)ncc34)C[C@@H]2CC#N)nc(C)n1] | | 297 | | +++ | | 3.89 | 29.68 |
| 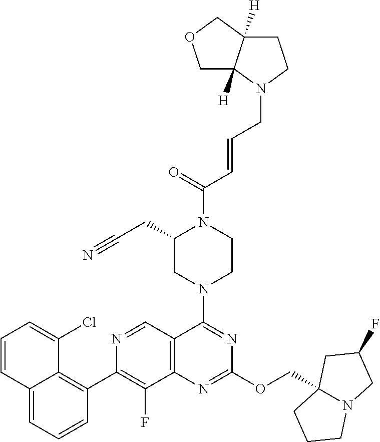 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@@H]2COC[C@H]21] | | 298 | | ++++ | | 2.99 | 117.56 |
| 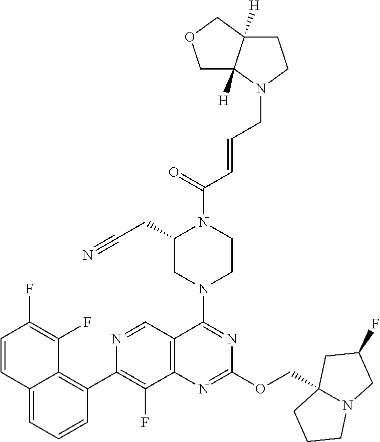 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CC[C@@H]2COC[C@H]21] | | 299 | | ++++ | | 2.95 | 117.58 |
| 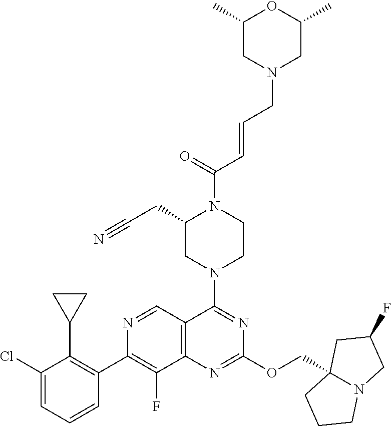 [SMILES: C[C@@H]1CN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)C[C@H](C)O1] | | 300 | | +++ | | 4.05 | 124.49 |
| 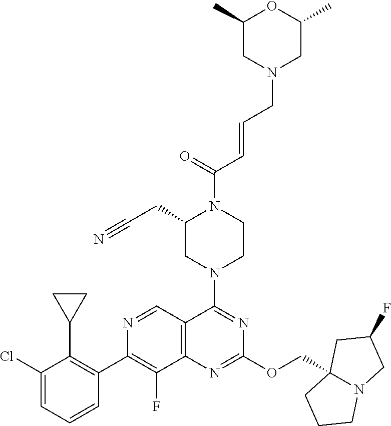 [SMILES: C[C@@H]1CN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)C[C@@H](C)O1] | | 301 | | +++ | | | |
| 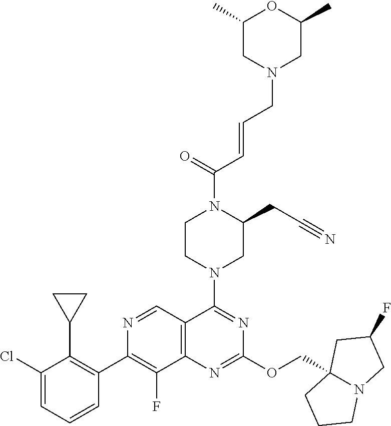 [SMILES: C[C@H]1CN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)C[C@H](C)O1] | | 302 | | ++++ | | 4.21 | 117.74 |
| 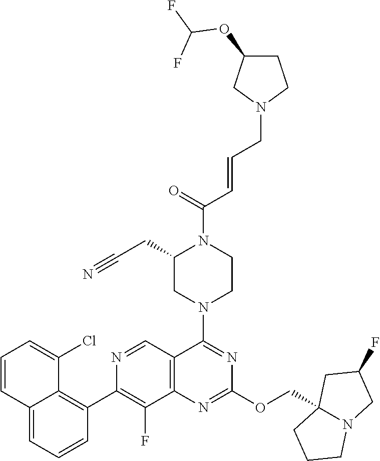 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@H](OC(F)F)C1] | | 303 | | ++++ | | | |
| 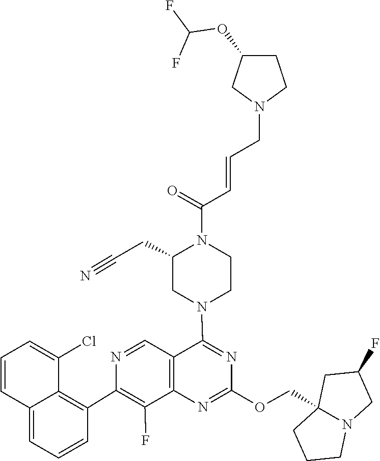 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@@H](OC(F)F)C1] | | 304 | | ++++ | | | |
| 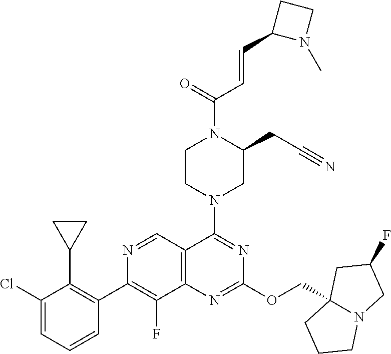 [SMILES: CN1CC[C@@H]1/C=C/C(=O)N1CCN(c2nc(OC[C@@]34CCCN3C[C@H](F)C4)nc3c(F)c(-c4cccc(Cl)c4C4CC4)ncc23)C[C@@H]1CC#N] | | 305 | | +++ | +++ | 2.87 | 130.46 |
| 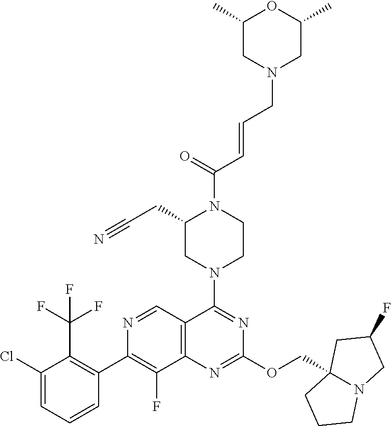 [SMILES: C[C@@H]1CN(C/C=C/C(=O)N2CCN(c3nc(OC[C@@]45CCCN4C[C@H](F)C5)nc4c(F)c(-c5cccc(Cl)c5C(F)(F)F)ncc34)C[C@@H]2CC#N)C[C@H](C)O1] | | 306 | | +++ | | 3.50 | 128.34 |
| 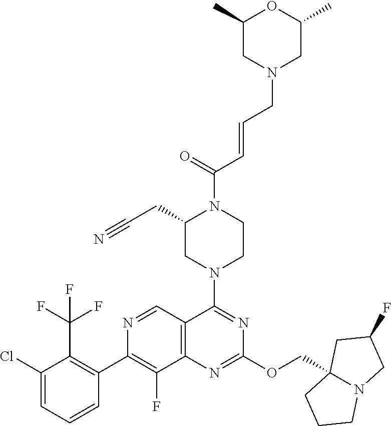 [SMILES: C[C@@H]1CN(C/C=C/C(=O)N2CCN(c3nc(OC[C@@]45CCCN4C[C@H](F)C5)nc4c(F)c(-c5cccc(Cl)c5C(F)(F)F)ncc34)C[C@@H]2CC#N)C[C@@H](C)O1] | | 307 | | ++++ | | 3.68 | 123.75 |
| 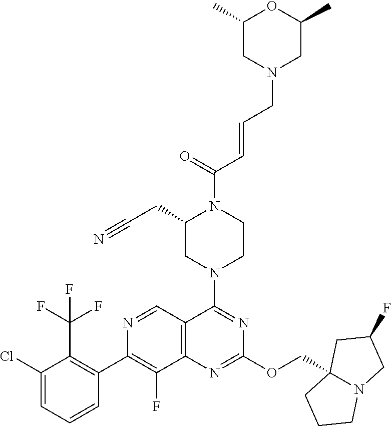 [SMILES: C[C@H]1CN(C/C=C/C(=O)N2CCN(c3nc(OC[C@@]45CCCN4C[C@H](F)C5)nc4c(F)c(-c5cccc(Cl)c5C(F)(F)F)ncc34)C[C@@H]2CC#N)C[C@H](C)O1] | | 308 | | +++ | | 3.75 | 80.55 |
| 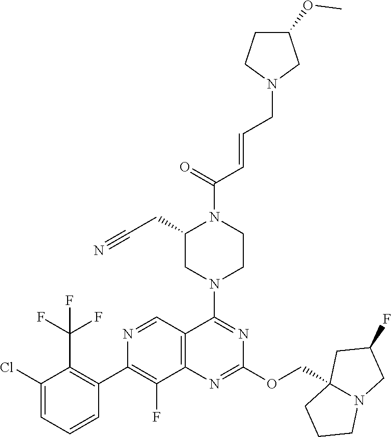 [SMILES: CO[C@H]1CCN(C/C=C/C(=O)N2CCN(c3nc(OC[C@@]45CCCN4C[C@H](F)C5)nc4c(F)c(-c5cccc(Cl)c5C(F)(F)F)ncc34)C[C@@H]2CC#N)C1] | | 309 | | +++ | | 2.79 | 141.39 |
| 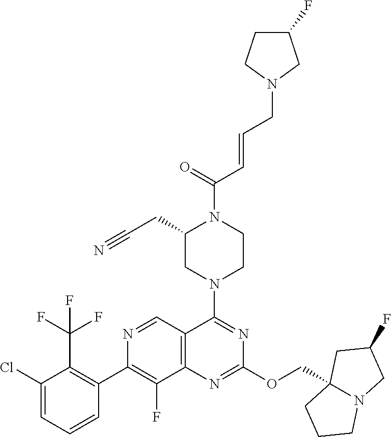 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CC[C@H](F)C1] | | 310 | | +++ | | 3.13 | 123.13 |
| 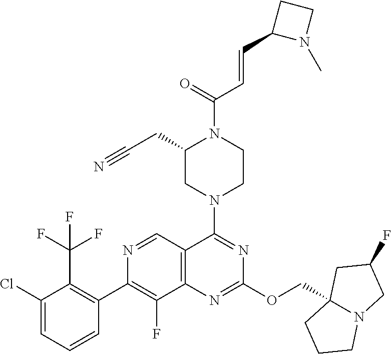 [SMILES: CN1CC[C@@H]1/C=C/C(=O)N1CCN(c2nc(OC[C@@]34CCCN3C[C@H](F)C4)nc3c(F)c(-c4cccc(Cl)c4C(F)(F)F)ncc23)C[C@@H]1CC#N] | | 311 | | ++ | | 2.54 | 127.97 |
| 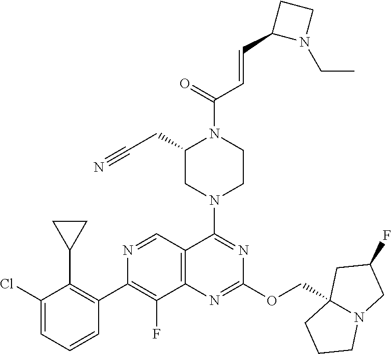 [SMILES: CCN1CC[C@@H]1/C=C/C(=O)N1CCN(c2nc(OC[C@@]34CCCN3C[C@H](F)C4)nc3c(F)c(-c4cccc(Cl)c4C4CC4)ncc23)C[C@@H]1CC#N] | | 312 | | +++ | | | |
| 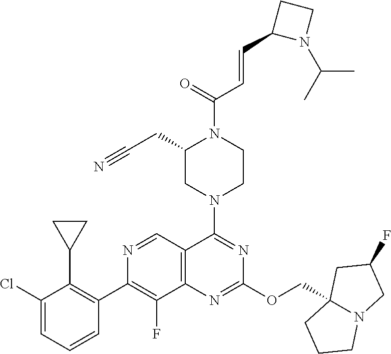 [SMILES: CC(C)N1CC[C@@H]1/C=C/C(=O)N1CCN(c2nc(OC[C@@]34CCCN3C[C@H](F)C4)nc3c(F)c(-c4cccc(Cl)c4C4CC4)ncc23)C[C@@H]1CC#N] | | 313 | | ++ | | | |
| 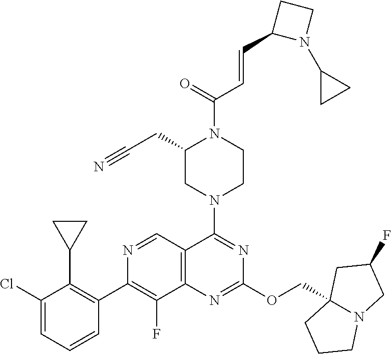 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/[C@H]1CCN1C1CC1] | | 314 | | ++++ | | 4.05 | 32.26 |
| 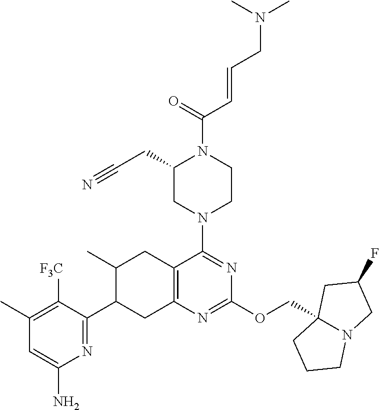 [SMILES: Cc1cc(N)nc(C2Cc3nc(OC[C@@]45CCCN4C[C@H](F)C5)nc(N4CCN(C(=O)/C=C/CN(C)C)[C@@H](CC#N)C4)c3CC2C)c1C(F)(F)F] | | 315 | | ++++ | ++++ | 2.47 | 138.84 |
| 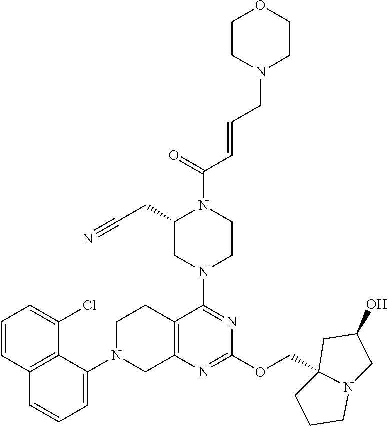 [SMILES: N#CC[C@H]1CN(c2nc(OC[C@@]34CCCN3C[C@H](O)C4)nc3c2CCN(c2cccc4cccc(Cl)c24)C3)CCN1C(=O)/C=C/CN1CCOCC1] | | 316 | | ++++ | | 2.66 | 123.51 |
| 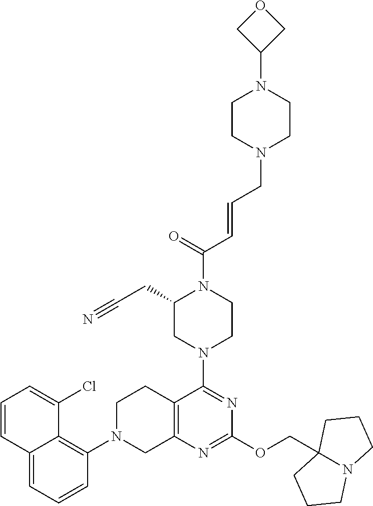 [SMILES: N#CC[C@H]1CN(c2nc(OCC34CCCN3CCC4)nc3c2CCN(c2cccc4cccc(Cl)c24)C3)CCN1C(=O)/C=C/CN1CCN(C2COC2)CC1] | | 317 | | ++++ | | 2.01 | 113.66 |
| 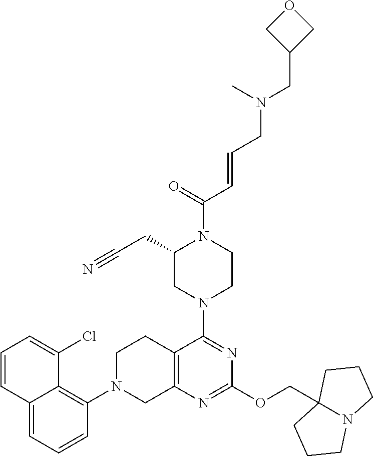 [SMILES: CN(C/C=C/C(=O)N1CCN(c2nc(OCC34CCCN3CCC4)nc3c2CCN(c2cccc4cccc(Cl)c24)C3)C[C@@H]1CC#N)CC1COC1] | | 318 | | ++++ | ++++ | | |
| 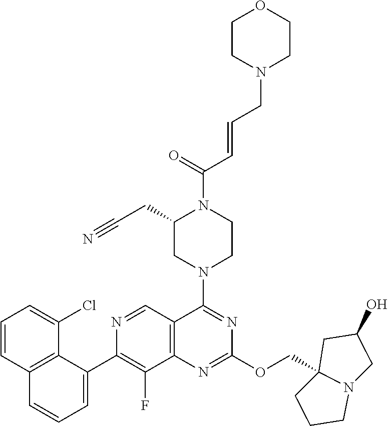 [SMILES: N#CC[C@H]1CN(c2nc(OC[C@@]34CCCN3C[C@H](O)C4)nc3c(F)c(-c4cccc5cccc(Cl)c45)ncc23)CCN1C(=O)/C=C/CN1CCOCC1] | | 319 | | ++++ | | 1.54 | 122.80 |
| 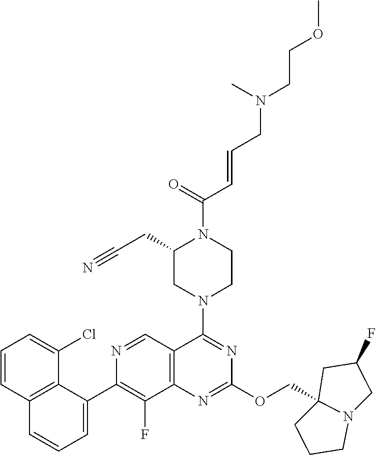 [SMILES: COCCN(C)C/C=C/C(=O)N1CCN(c2nc(OC[C@@]34CCCN3C[C@H](F)C4)nc3c(F)c(-c4cccc5cccc(Cl)c45)ncc23)C[C@@H]1CC#N] | | 320 | | ++++ | ++++ | 3.05 | 103.64 |
| 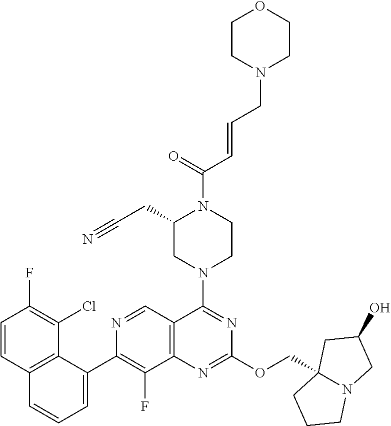 [SMILES: N#CC[C@H]1CN(c2nc(OC[C@@]34CCCN3C[C@H](O)C4)nc3c(F)c(-c4cccc5ccc(F)c(Cl)c45)ncc23)CCN1C(=O)/C=C/CN1CCOCC1] | | 321 | | ++++ | | 1.52 | 162.29 |
| 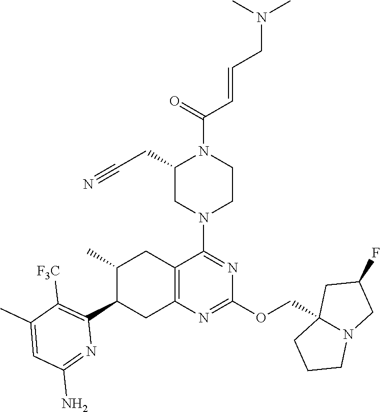 [SMILES: Cc1cc(N)nc([C@@H]2Cc3nc(OC[C@@]45CCCN4C[C@H](F)C5)nc(N4CCN(C(=O)/C=C/CN(C)C)[C@@H](CC#N)C4)c3C[C@H]2C)c1C(F)(F)F] | | 322 | | ++++ | ++++ | 2.79 | 132.54 |
| 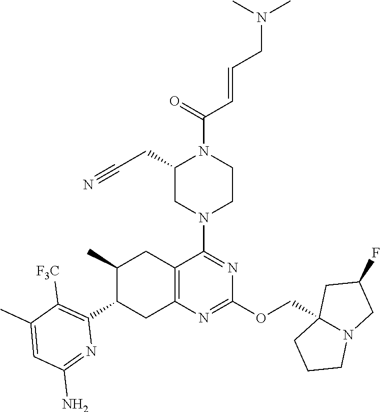 [SMILES: Cc1cc(N)nc([C@H]2Cc3nc(OC[C@@]45CCCN4C[C@H](F)C5)nc(N4CCN(C(=O)/C=C/CN(C)C)[C@@H](CC#N)C4)c3C[C@@H]2C)c1C(F)(F)F] | | 323 | | ++ | | 2.71 | 74.51 |
| 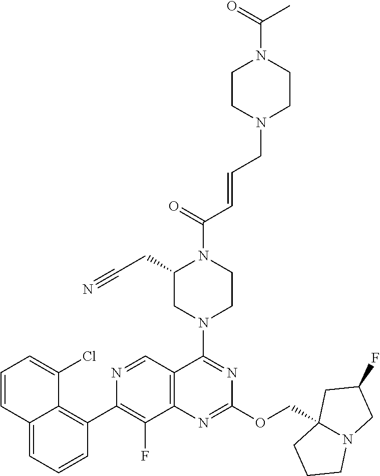 [SMILES: CC(=O)N1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)CC1] | | 324 | | ++++ | | | |
| 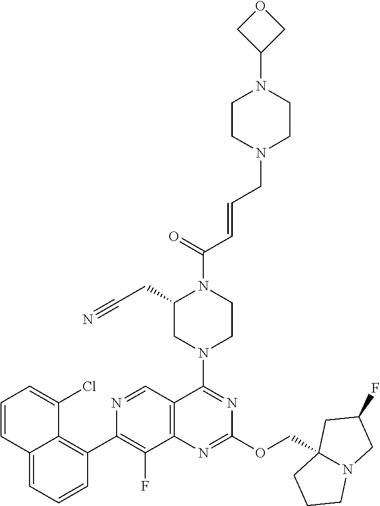 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCN(C2COC2)CC1] | | 325 | | ++++ | | 2.50 | 114.87 |
| 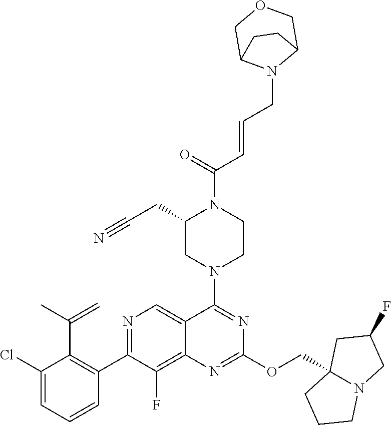 [SMILES: C=C(C)c1c(Cl)cccc1-c1ncc2c(N3CCN(C(=O)/C=C/CN4C5CCC4COC5)[C@@H](CC#N)C3)nc(OC[C@@]34CCCN3C[C@H](F)C4)nc2c1F] | | 326 | | ++++ | | | |

* ++++ is less than 10 nM, +++ is 10 to less than 100 nM, ++ is 100 to less than 500 nM, + is greater or equal to 500 nM

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A compound of Formula (A), Formula (B) or Formula (C) 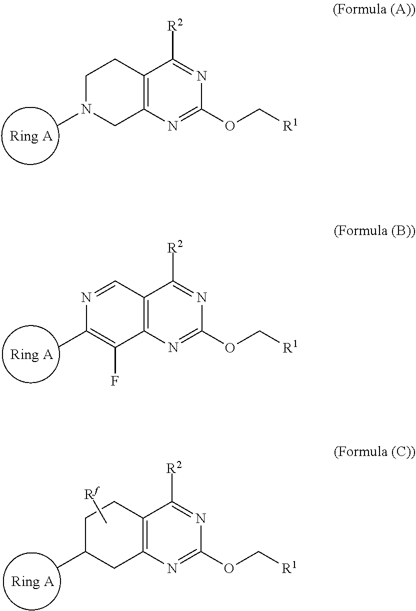
or a salt thereof; and/or an isotopologue thereof, wherein:
Ring A is a 6-10 membered aryl or a 5-10 membered heteroaryl, each substituted with 0, 1, 2 or 3 substituents independently selected from halo, —OH, —NH₂, C₁-C₄ alkyl, C₁-C₄ alkenyl, C₁-C₄ alkoxy, C₁-C₄ haloalkyl, C₁-C₄ haloalkoxy and C₂-C₃ alkynyl;
R¹ is 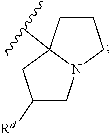
R^{d} is H, —F or —OH;
R² is R^{2c};
R^{2c} is 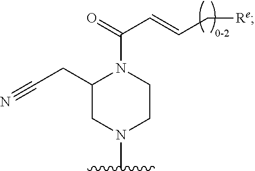
R^{e} is R^{e1}, R^{e2} or R^{e3};
R^{e1} is a 4-10 membered heterocycle which is substituted with 0, 1, 2, 3 or 4 substituents independently selected from halo, hydroxy, C₁-C₄ alkyl, C₁-C₆ aminoalkyl, 4-6 membered heterocycle, —C(O)C₁-C₆ alkyl, C₁-C₆ alkoxy, C₁-C₆ alkoxyalkoxy, C₁-C₄ haloalkyl, C₁-C₄ haloalkoxy and C₂-C₃ alkynyl;
R^{e2} is a 5-6 membered heteroaryl wherein the attachment point is a carbon atom group and wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl;
R^{e3} is —NR⁵R⁶, wherein each R⁵ and R⁶ is independently selected from C₁-C₄ alkyl, C₁-C₆ alkoxy and —CH₂-(4-6 membered heterocycle); and
R^{f} is selected from the group consisting of H, halo, C₁-C₄ alkyl, C₁-C₄ alkoxy, C₁-C₄ haloalkyl and C₁-C₄ haloalkoxy.

2. The compound of claim 1, wherein R^{2c} is selected from the group consisting of:

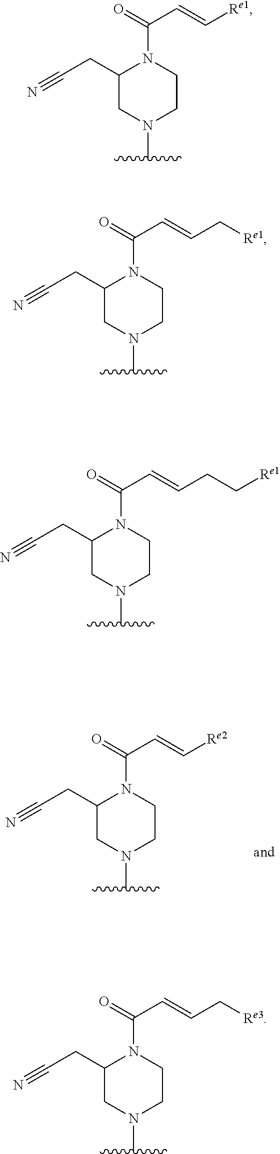

3. The compound of claim 1, wherein R^{2c} is selected from the group consisting of:

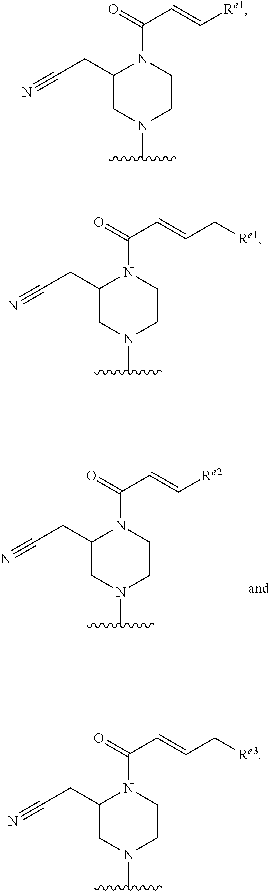

4. The compound of any one of claims 1 to 3, wherein the compound is of Formula (A).

5. The compound of any one of claims 1 to 3, wherein the compound is of Formula (B).

6. The compound of any one of claims 1 to 3, wherein the compound is of Formula (C).

7. The compound of any one of claims 1 to 6, wherein Ring A is selected from the group consisting of naphthalenyl, phenyl, isoquinolinyl, pyridinyl and 1-H-indazolyl, each substituted with 0, 1, 2 or 3 substituents independently selected from halo, —OH, —NH₂, C₁-C₄ alkyl, C₁-C₄ alkoxy, C₁-C₄ alkenyl, C₁-C₄ haloalkyl, C₁-C₄ haloalkoxy and C₂-C₃ alkynyl.

8. The compound of claim 7, wherein the naphthalenyl, phenyl, isoquinolinyl, pyridinyl and 1-H-indazolyl are independently substituted with 0, 1, 2 or 3 substituents independently selected from halo, —NH₂, C₁-C₄ acyclic alkyl, C₃-C₄ cycloalkyl, C₁-C₄ alkenyl, C₁-C₄ haloalkyl and C₂-C₃ alkynyl.

9. The compound of claim 7, wherein the naphthalenyl, phenyl, isoquinolinyl, pyridinyl and 1-H-indazolyl are independently substituted with 0, 1, 2 or 3 substituents independently selected from —F, —Cl, —OH, —NH₂, -Me, -Et, —Pr, -ⁱPr, cyclopropyl, cyclobutyl, vinyl, prop-1-en-2-yl, —CHF₂, —CH₂F, —CF₃, —OMe, -OEt, —OCHF₂, —OCF₃ and ethynyl.

10. The compound of claim 7, wherein the naphthalenyl, phenyl, isoquinolinyl, pyridinyl and 1-H-indazolyl are independently substituted with 0, 1, 2 or 3 substituents independently selected from —F, —Cl, —NH₂, -Me, -Et, cyclopropyl, cyclobutyl, prop-1-en-2-yl, —CHF₂, —CF₃, and ethynyl.

11. The compound of any one of claims 1 to 6, wherein Ring A is selected from the group consisting of 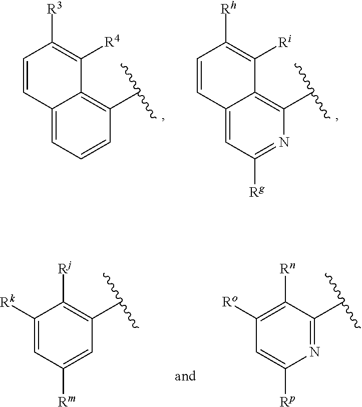
wherein:
each R³, R⁴, R^{h}, Rⁱ, R^{j}, R^{k}, Rⁿ and R^{o} is independently selected from the group consisting of hydrogen, halo, C₁-C₄ alkyl, C₁-C₄ alkoxy, C₁-C₄ haloalkyl, C₁-C₄ haloalkoxy, C₁-C₄ alkenyl and C₂-C₃ alkynyl; and
each R^{g}, R^{m} and R^{p} is independently selected from the group consisting of hydrogen, halo, —NH₂, C₁-C₄ alkyl, C₁-C₄ alkoxy, C₁-C₄ haloalkyl, C₁-C₄ haloalkoxy and C₂-C₃ alkynyl.

12. The compound of claim 11, wherein Ring A is:

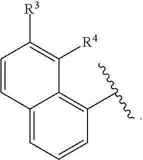

13. The compound of claim 11, wherein Ring A is:

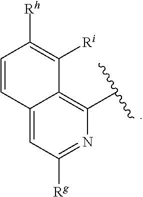

14. The compound of claim 11, wherein Ring A is:

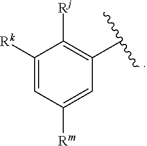

15. The compound of claim 11, wherein Ring A is:

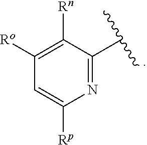

16. The compound of any one of claims 1 to 6, wherein Ring A is selected from the group consisting of:

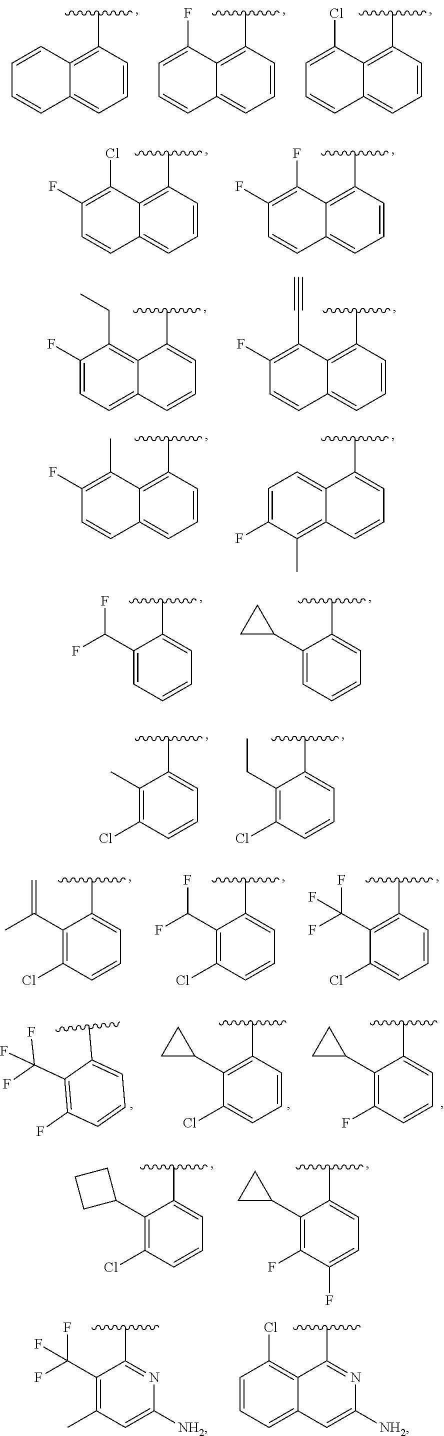

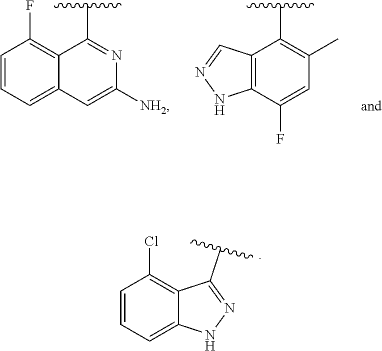 [SMILES: *c1nc(N)cc2cccc(F)c12 ; *c1c(C)cc(F)c2[nH]ncc12 ; "and" ; *c1n[nH]c2cccc(Cl)c12]

17. The compound of any one of claims 1 to 6, wherein Ring A is selected from the group consisting of:

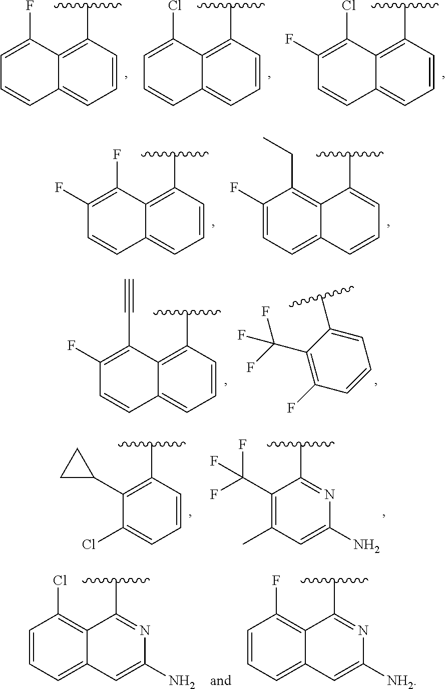

18. The compound of any one of claims 1 to 6, wherein Ring A is naphthalenyl substituted with 0, 1, 2 or 3 substituents independently selected from halo, hydroxy, C₁-C₄ alkyl, C₁-C₄ alkoxy, C₁-C₄ haloalkyl, C₁-C₄ haloalkoxy and C₂-C₃ alkynyl.

19. The compound of claim 18, wherein the naphthalenyl is substituted with 0, 1 or 2 substituents independently selected from halo, C₁-C₄ acyclic alkyl and C₂-C₃ alkynyl.

20. The compound of claim 18, wherein the naphthalenyl is substituted with 0, 1 or 2 substituents independently selected from —F, —Cl, -Me, -Et and ethynyl.

21. The compound of claim 18, wherein the naphthalenyl is substituted with 1 or 2 instances of —Cl.

22. The compound of any one of claims 18 to 21, wherein the naphthalenyl is naphthalen-1-yl.

23. The compound of any one of claims 1 to 6, wherein Ring A is selected from the group consisting of:

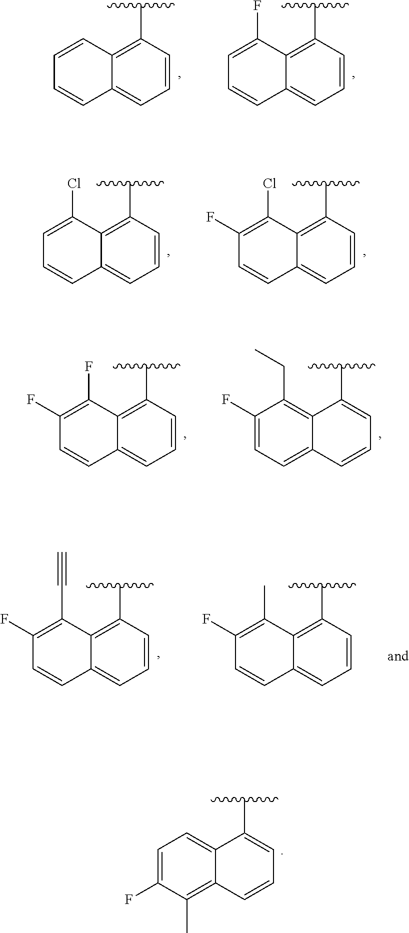

24. The compound of any one of claims to 6, wherein Ring A is selected from the group consisting of:

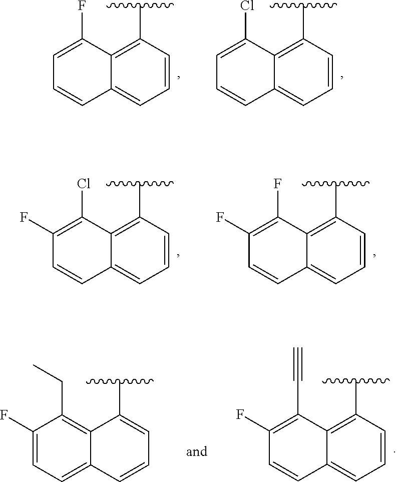 [SMILES: *c1cccc2cccc(F)c12 ; *c1cccc2cccc(Cl)c12 ; *c1cccc2ccc(F)c(Cl)c12 ; *c1cccc2ccc(F)c(F)c12 ; *c1cccc2ccc(F)c(CC)c12 ; *c1cccc2ccc(F)c(C#C)c12]

25. The compound of claim 1, wherein the compound is of Formula I or Formula II: 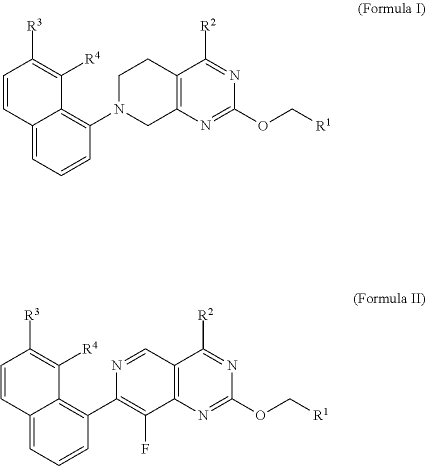
or a salt thereof, and/or an isotopologue thereof, wherein:
R³ is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl; and
R⁴ is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl.

26. The compound of claim 25, wherein the compound is of Formula I.

27. The compound of claim 25, wherein the compound is of Formula II.

28. The compound of any one of claims 11, 12 and 25 to 27, wherein R³ is selected from the group consisting of hydrogen and —F.

29. The compound of any one of claims 11, 12 and 25 to 28, wherein R⁴ is selected from the group consisting of hydrogen, —F, —Cl, -Et and ethynyl.

30. The compound of any one of claims 11, 12 and 25 to 28, wherein R⁴ is selected from the group consisting of —F and —Cl.

31. The compound of claim 1, wherein Ring A is isoquinolinyl substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl.

32. The compound of claim 31, wherein the isoquinolinyl is substituted with 1 or 2 substituents independently selected from —F, —C₁ and —NH₂.

33. The compound of claim 31 or 32, wherein the isoquinolinyl is isoquinolinyl-1-yl.

34. The compound of any one of claims 1 to 6, wherein Ring A is selected from the group consisting of

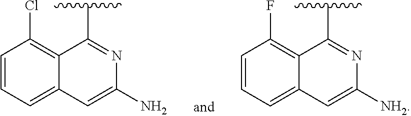 [SMILES: *c1nc(N)cc2cccc(Cl)c12 ; *c1nc(N)cc2cccc(F)c12]

35. The compound of claim 1, wherein the compound is of Formula III or Formula IV: 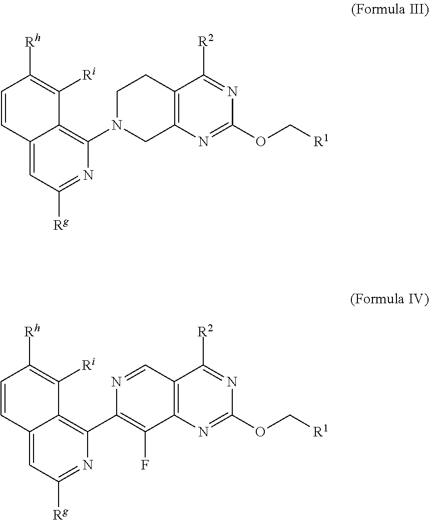
or a salt thereof, and/or an isotopologue thereof, wherein:
R^{h} is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl;
Rⁱ is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl; and
R^{g} is selected from the group consisting of hydrogen, halo, —NH₂, C₁-C₄ alkyl, C₁-C₄ alkoxy, C₁-C₄ haloalkyl, C₁-C₄ haloalkoxy and C₂-C₃ alkynyl.

36. The compound of claim 35, wherein the compound is of Formula III.

37. The compound of claim 35, wherein the compound is of Formula IV.

38. The compound of any one of claims 11, 13 and 35 to 37, wherein R^{h} is selected from the group consisting of hydrogen and —F.

39. The compound of any one of claims 11, 13 and 35 to 37, wherein R^{h} is hydrogen.

40. The compound of any one of claims 11, 13 and 35 to 39, wherein Rⁱ is selected from the group consisting of —F and —Cl.

41. The compound of any one of claims 11, 13 and 35 to 40, wherein R^{g} is —NH₂.

42. The compound of any one of claims 1 to 6, wherein Ring A is phenyl substituted with 0, 1, 2 or 3 substituents independently selected from halo, —OH, —NH₂, C₁-C₄ alkyl, C₁-C₄ alkenyl, C₁-C₄ alkoxy, C₁-C₄ haloalkyl, C₁-C₄ haloalkoxy and C₂-C₃ alkynyl.

43. The compound of claim 42, wherein the phenyl is substituted with 1, 2 or 3 substituents independently selected from —F, —Cl, -Me, -Et, cyclopropyl, cyclobutyl, prop-1-en-2-yl, —CHF₂ and —CF₃.

44. The compound of any one of claims 1 to 6, wherein Ring A is selected from the group consisting of

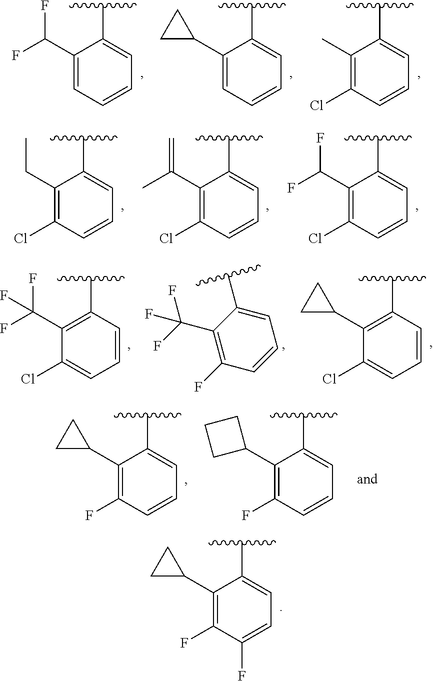

45. The compound of any one of claims 1 to 6, wherein Ring A is selected from the group consisting of

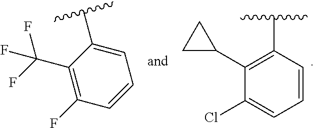 [SMILES: *c1cccc(F)c1C(F)(F)F ; *c1cccc(Cl)c1C1CC1]

46. The compound of claim 1, wherein the compound is of Formula V or Formula VI: 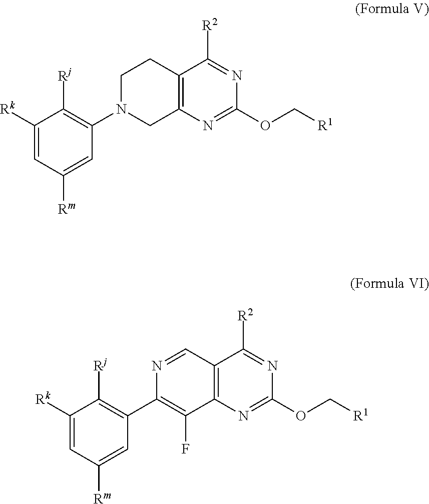
or a salt thereof, and/or an isotopologue thereof, wherein:
R^{j} is selected from the group consisting of hydrogen, halo, C₁-C₄ alkyl, C₁-C₄ alkoxy, C₁-C₄ haloalkyl, C₁-C₄ alkenyl, C₁-C₄ haloalkoxy and C₂-C₃ alkynyl;
R^{k} is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl;
R^{m} is selected from the group consisting of hydrogen, halo, —NH₂, C₁-C₄ alkyl, C₁-C₄ alkoxy, C₁-C₄ haloalkyl, C₁-C₄ haloalkoxy and C₂-C₃ alkynyl.

47. The compound of claim 46, wherein the compound is of Formula V.

48. The compound of claim 46, wherein the compound is of Formula VI.

49. The compound of any one of claims 11, 14 and 46 to 48, wherein R^{j} is selected from the group consisting of C₁-C₄ acyclic alkyl, C₃-C₄ cycloalkyl, C₁-C₄ alkenyl and C₁-C₄ haloalkyl.

50. The compound of any one of claims 11, 14 and 46 to 48, wherein R^{j} is selected from the group consisting of C₃-C₄ cycloalkyl and C₁-C₄ haloalkyl.

51. The compound of any one of claims 11, 14 and 46 to 48, wherein R^{j} is selected from the group consisting of -Me, -Et, cyclopropyl, cyclobutyl, prop-1-en-2-yl, —CHF₂ and —CF₃.

52. The compound of any one of claims 11, 14 and 46 to 48, wherein R^{j} is selected from the group consisting of cyclopropyl and —CF₃.

53. The compound of any one of claims 11, 14 and 46 to 52, wherein R^{k} is selected from the group consisting of hydrogen and halo.

54. The compound of any one of claims 11, 14 and 46 to 52, wherein R^{k} is selected from the group consisting of hydrogen, —F and —Cl.

55. The compound of any one of claims 11, 14 and 46 to 54, wherein R^{k} is hydrogen.

56. The compound of any one of claims 1 to 6, wherein Ring A is pyridinyl substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl.

57. The compound of claim 56, wherein the pyridinyl is substituted with 1, 2 or 3 substituents independently selected from —NH₂, -Me and —CF₃.

58. The compound of any one of claims 1 to 6, wherein Ring A is

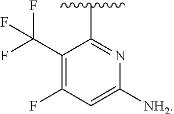 [SMILES: *c1nc(N)cc(F)c1C(F)(F)F]

59. The compound of any one of claims 1 to 6, wherein the compound is of Formula VII, Formula VIII or Formula IX: 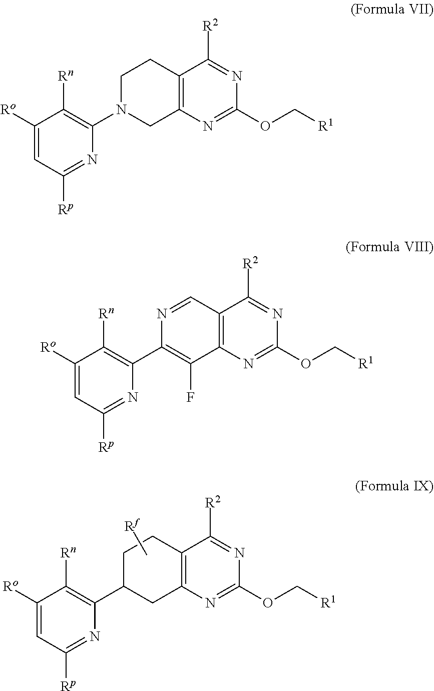
or a salt thereof, and/or an isotopologue thereof, wherein:
Rⁿ is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl;
R^{o} is selected from the group consisting of hydrogen, halo, C₁-C₄ alkyl, C₁-C₄ alkoxy, C₁-C₄ haloalkyl, C₁-C₄ haloalkoxy and C₂-C₃ alkynyl;
R^{p} is selected from the group consisting of hydrogen, halo, —NH₂, C₁-C₄ alkyl, C₁-C₄ alkoxy, C₁-C₄ haloalkyl, C₁-C₄ haloalkoxy and C₂-C₃ alkynyl.

60. The compound of claim 59, wherein the compound is of Formula VII.

61. The compound of claim 59, wherein the compound is of Formula VIII.

62. The compound of claim 59, wherein the compound is of Formula IX.

63. The compound of claim 59, wherein the compound is of Formula IXa: 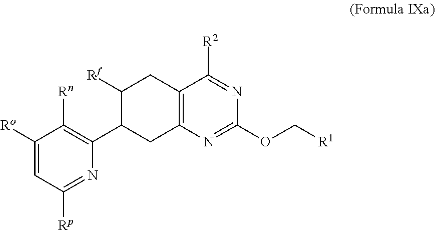
or a salt thereof, and/or an isotopologue thereof.

64. The compound of any one of claims 11, 15 and 59 to 63, wherein Rⁿ is trifluoromethyl.

65. The compound of any one of claims 11, 15 and 59 to 64, wherein R^{o} is methyl or fluoro.

66. The compound of any one of claims 11, 15 and 59 to 65, wherein R^{p} is —NH₂.

67. The compound of any one of claims 1, 6-22, 31-34, 42-45 and 56 to 66, wherein R^{f} is methyl.

68. The compound of any one of claims 1 to 6, wherein Ring A is 1-H-indazolyl substituted with 0, 1, 2 or 3 substituents independently selected from halo, —OH, —NH₂, C₁-C₄ alkyl, C₁-C₄ alkoxy, C₁-C₄ haloalkyl, C₁-C₄ haloalkoxy and C₂-C₃ alkynyl.

69. The compound of claim 68, wherein Ring A is selected from

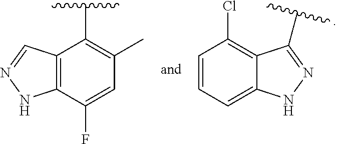 [SMILES: *c1c(C)cc(F)c2[nH]ncc12 ; *c1n[nH]c2cccc(Cl)c12]

70. The compound of any one of claims 1 to 69, wherein R^{d} is selected from the group consisting of H and —F.

71. The compound of any one of claims 1 to 69, wherein R^{d} is F.

72. The compound of any one of claims 1 to 71, wherein R^{2c} is selected from the group consisting of

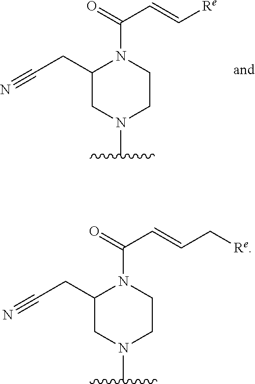

73. The compound of any one of claims 1 to 72, wherein R^{e} is R^{e1}.

74. The compound of any one of claims 1 to 73, wherein R^{e1} is a 4-7 membered monocyclic heterocycle containing a nitrogen or an oxygen atom as the only heteroatom, wherein the monocyclic heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

75. The compound of claim 74, wherein the monocyclic heterocycle of R is substituted with 0 or 1 instances of C₁-C₄ alkyl.

76. The compound of claim 74, wherein the monocyclic heterocycle of R^{e1} is substituted with 0 or 1 instances of -Me, -ⁱPr or cyclopropyl.

77. The compound of claim 74, wherein R^{e1} is selected from the group consisting of azetidinyl and oxetanyl, each substituted with 0 or 1 substituents independently selected from -Me, -ⁱPr or cyclopropyl.

78. The compound of any one of claims 74-77, wherein the attachment point for the monocyclic heterocycle is on a carbon atom.

79. The compound of claim 74, wherein R^{e} is selected from the group consisting of:

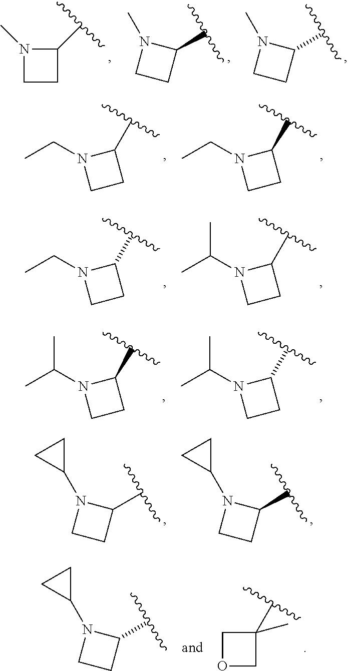

80. The compound of any one of claims 74 to 79, wherein R^{2c} is

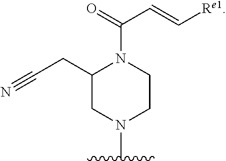

81. The compound of any one of claims 1 to 73, wherein R^{e1} is a 4-10 membered heterocycle containing a nitrogen atom and one or two additional heteroatoms selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

82. The compound of any one of claims 1 to 73, wherein R^{e1} is a 4-10 membered heterocycle containing a nitrogen atom and one or two additional heteroatoms selected from oxygen and sulfur, including sulfur dioxide, selected from the group consisting of a 4-8 member monocyclic heterocycle, a 6-10 member fused bicyclic heterocycle, a 6-10 member bridged heterocycle and a 6-10 member spiro heterocycle, each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

83. The compound of any one of claims 1 to 73, wherein R is selected from azetidine, pyrrolidine, isothiazolidine 1,1-dioxide, 3-azabicyclo[3.1.0]hexane, piperidine, piperazine, 2-oxabicyclo[4.1.0]heptane, 3-oxa-6-azabicyclo[3.2.0]heptane, hexahydro-1H-furo[3,4-c]pyrrole, hexahydro-1H-furo[3,4-b]pyrrole, 2-azabicyclo[2.1.1]hexane, morpholine, 2-oxa-5-azabicyclo[4.1.0]heptane, 1, 4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 7-oxa-2-azabicyclo[3.3.1]nonane, 8-oxa-3-azabicyclo[3.2.1]octane, 9-oxa-3-azabicyclo[3.3.1]nonane, 9-oxa-3-azabicyclo[3.3.1]nonane, 2-oxa-6-azaspiro[3.3]heptane, 3-oxa-6-azabicyclo[3.1.1]heptane, 6-oxa-3-azabicyclo[3.1.1]heptane, 2-oxa-5-azabicyclo[2.2.2]octane, 5,6,7,8-tetrahydro-[1,2,4]triazolo[4,3-a]pyrazine, 5,6,7,8-tetrahydro-[1,2,4]triazolo[1,5-a]pyrazine, 4,5,6,7-tetrahydropyrazolo[1,5-a]pyrazine, 5,6,7,8-tetrahydroimidazo[1,2-a]pyrazine, thiomorpholine, thiomorpholine 1,1-dioxide, 4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

84. The compound of any one of claims 1 to 73, wherein R is selected from azetidine, pyrrolidine, isothiazolidine 1,1-dioxide, 3-azabicyclo[3.1.0]hexane, piperidine, piperazine, 2-oxabicyclo[4.1.0]heptane, 3-oxa-6-azabicyclo[3.2.0]heptane, hexahydro-1H-furo[3,4-c]pyrrole, hexahydro-1H-furo[3,4-b]pyrrole, morpholine, 2-oxa-5-azabicyclo[4.1.0]heptane, 1, 4-oxazepane, 2-oxa-5-azabicyclo[2.2.1]heptane, 3,9-dioxa-7-azabicyclo[3.3.1]nonane, 3-oxa-8-azabicyclo[3.2.1]octane, 8-oxa-3-azabicyclo[3.2.1]octane, 2-oxa-6-azaspiro[3.3]heptane, 3-oxa-6-azabicyclo[3.1.1]heptane, 6-oxa-3-azabicyclo[3.1.1]heptane, 2-oxa-5-azabicyclo[2.2.2]octane, 5,6,7,8-tetrahydro-[1,2,4]triazolo[4,3-a]pyrazine, 5,6,7,8-tetrahydro-[1,2,4]triazolo[1,5-a]pyrazine, 4,5,6,7-tetrahydropyrazolo[1,5-a]pyrazine, 5,6,7,8-tetrahydroimidazo[1,2-a]pyrazine, thiomorpholine and thiomorpholine 1,1-dioxide, each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

85. The compound of any one of claims 1 to 73, wherein R is selected from azetidine, pyrrolidine, piperidine, hexahydro-1H-furo[3,4-c]pyrrole, morpholine, 1, 4-oxazepane, 2-oxa-5-azabicyclo[2.2.1]heptane, 3-oxa-8-azabicyclo[3.2.1]octane, 8-oxa-3-azabicyclo[3.2.1]octane, 3-oxa-6-azabicyclo[3.1.1]heptane, 6-oxa-3-azabicyclo[3.1.1]heptane, 2-oxa-5-azabicyclo[2.2.2]octane and thiomorpholine, each substituted with 0, 1 or 2 substituents independently selected from —F, -Me, —OMe and -OEt.

86. The compound of any one of claims 1 to 73, wherein R is selected from hexahydro-1H-furo[3,4-c]pyrrole, morpholine, 1, 4-oxazepane, 2-oxa-5-azabicyclo[2.2.1]heptane, 3-oxa-8-azabicyclo[3.2.1]octane, 8-oxa-3-azabicyclo[3.2.1]octane, 3-oxa-6-azabicyclo[3.1.1]heptane, 6-oxa-3-azabicyclo[3.1.1]heptane, 2-oxa-5-azabicyclo[2.2.2]octane and thiomorpholine, each unsubstituted.

87. The compound of any one of claims 81 to 86, wherein the attachment point for R^{e} is the nitrogen atom of the heterocycle.

88. The compound of any one of claims 1 to 73, wherein the R^{e} is selected from the group consisting of: 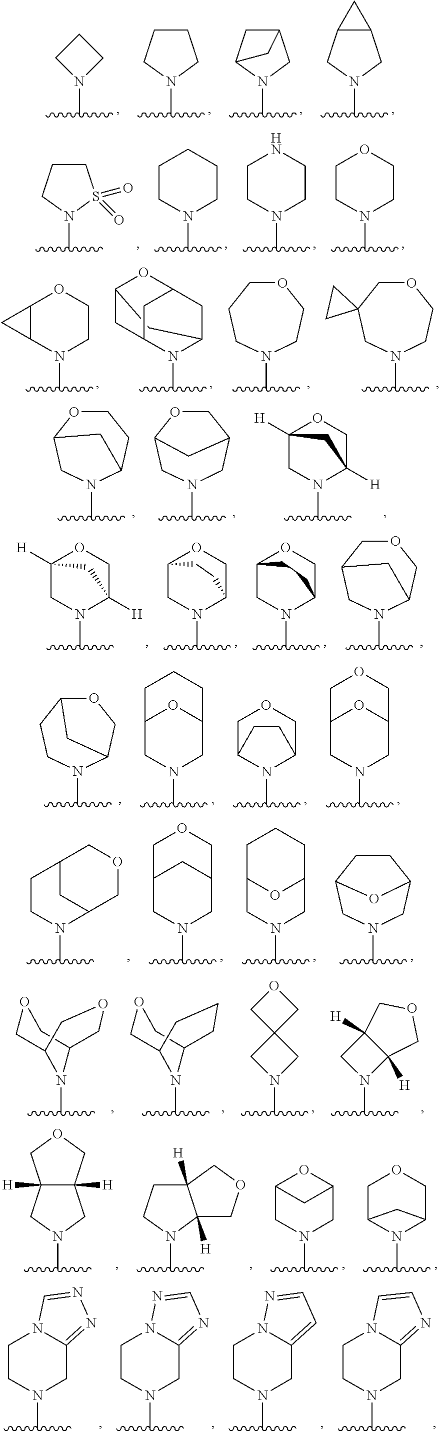 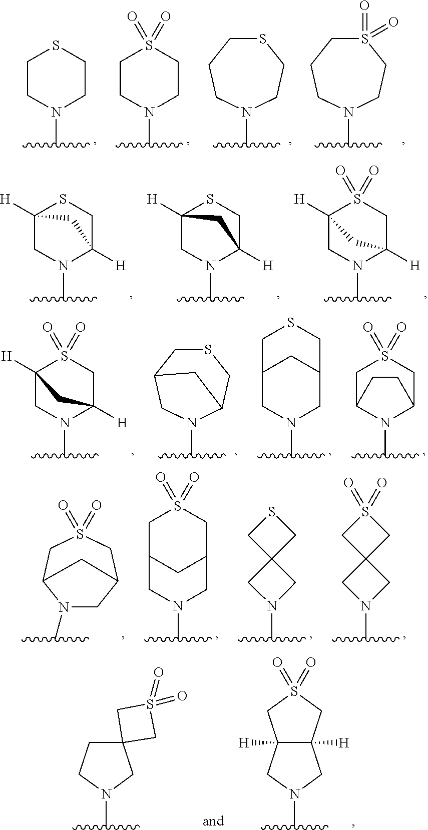
each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

89. The compound of of any one of claims 1 to 73, wherein the R is selected from the group consisting of: 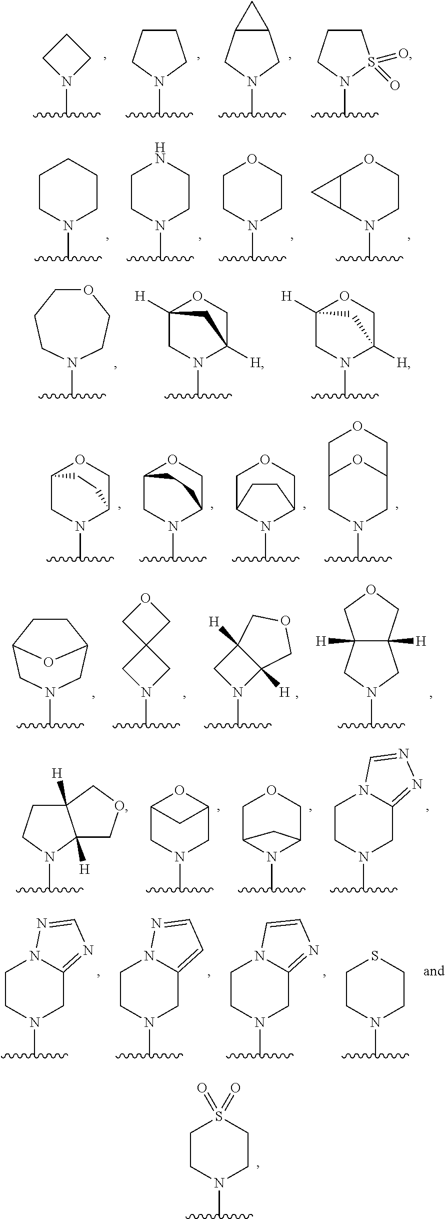
each substituted with 0, 1, 2, 3 or 4 substituents independently selected from halo, hydroxy, C₁-C₄ alkyl, 4-6 membered heterocycle, —C(O)C₁-C₆ alkyl, C₁-C₆ aminoalkyl, C₁-C₆ alkoxy, C₁-C₆ alkoxyalkoxy, C₁-C₄ haloalkyl, C₁-C₄ haloalkoxy and C₂-C₃ alkynyl.

90. The compound of any one of claims 1 to 73, wherein R^{e1} is selected from 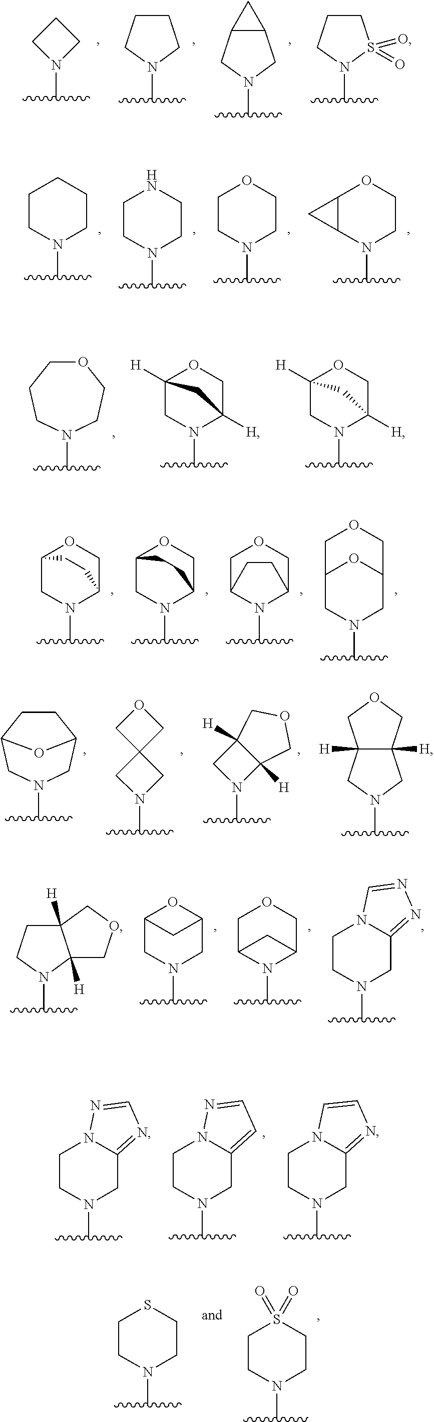
each substituted with 0, 1 or 2 substituents independently selected from —F, -Me, —OMe, -OEt, —OCHF₂, —C(═O)Me and oxetanyl.

91. The compound of any one of claims 81 to 90, wherein the 4-10 membered heterocycle of R^{e1} is unsubstituted.

92. The compound of any one of claims 1 to 73, wherein R is selected from, 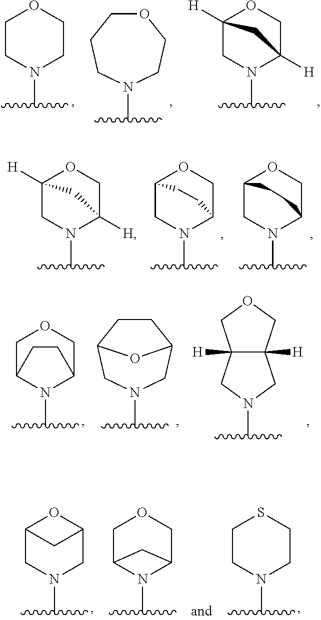
each unsubstituted.

93. The compound of any one of claims 1 to 73, wherein R is selected from the group consisting of:

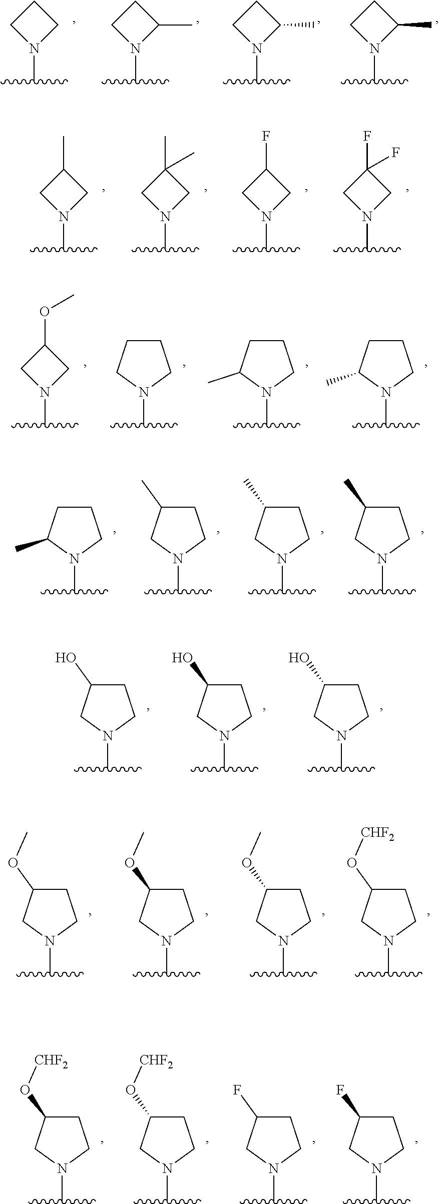

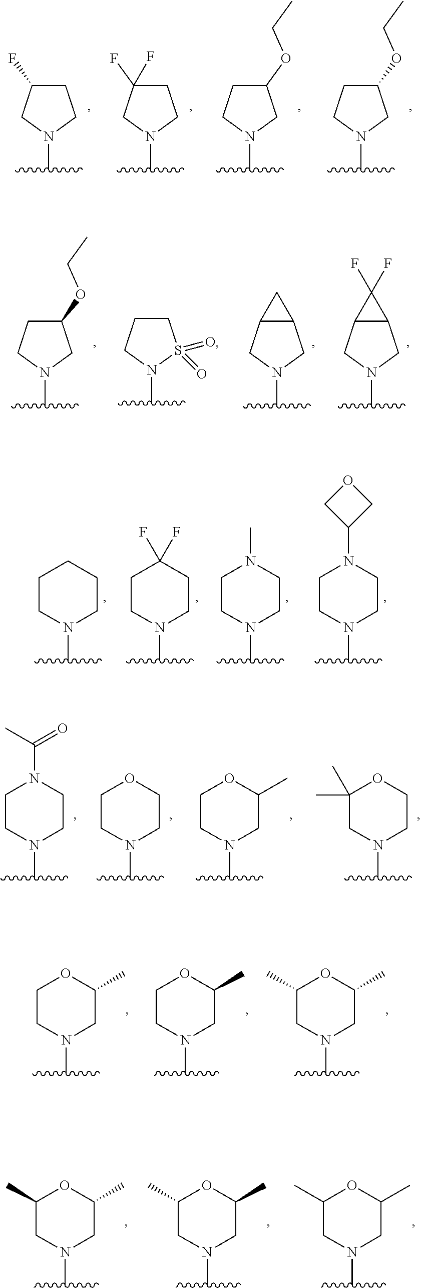

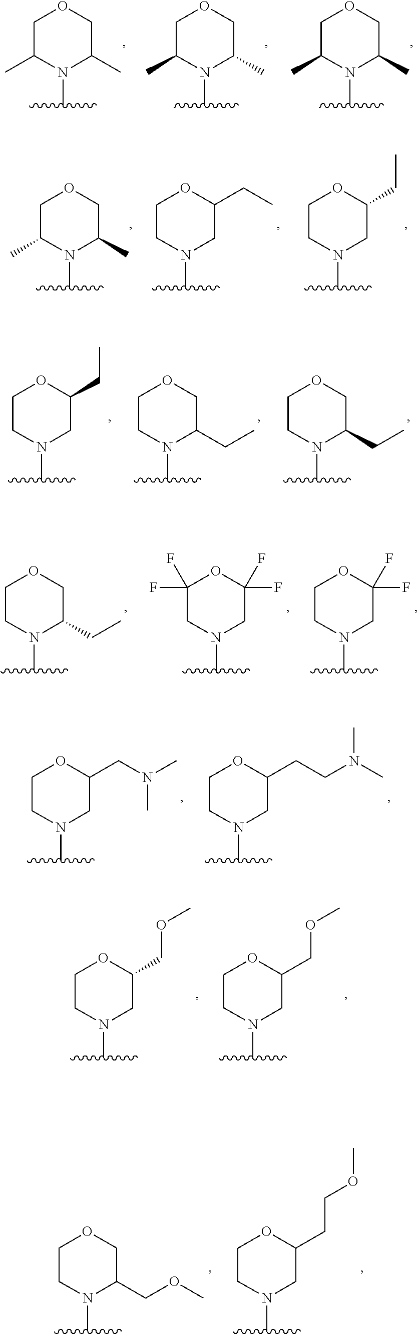

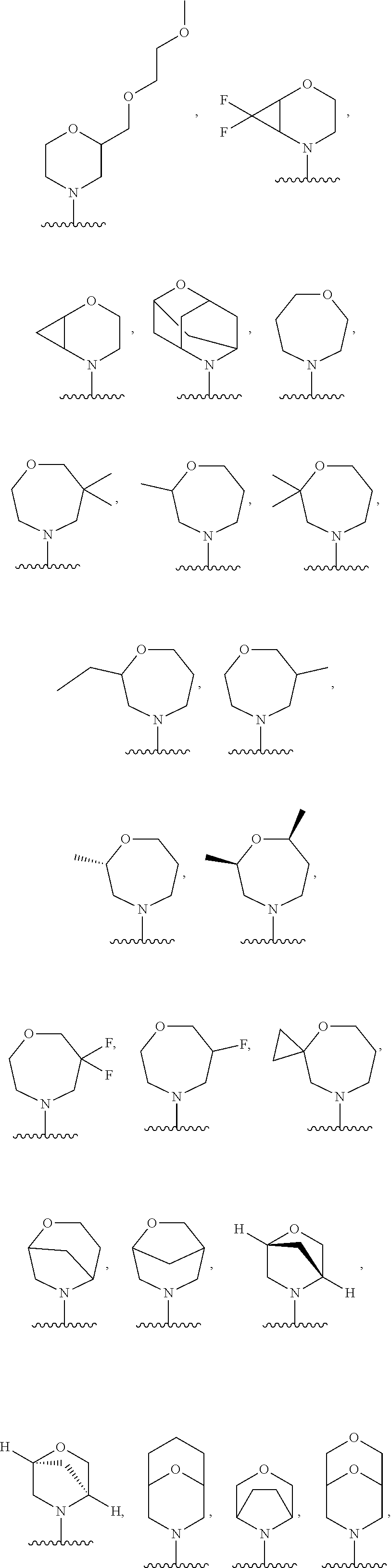

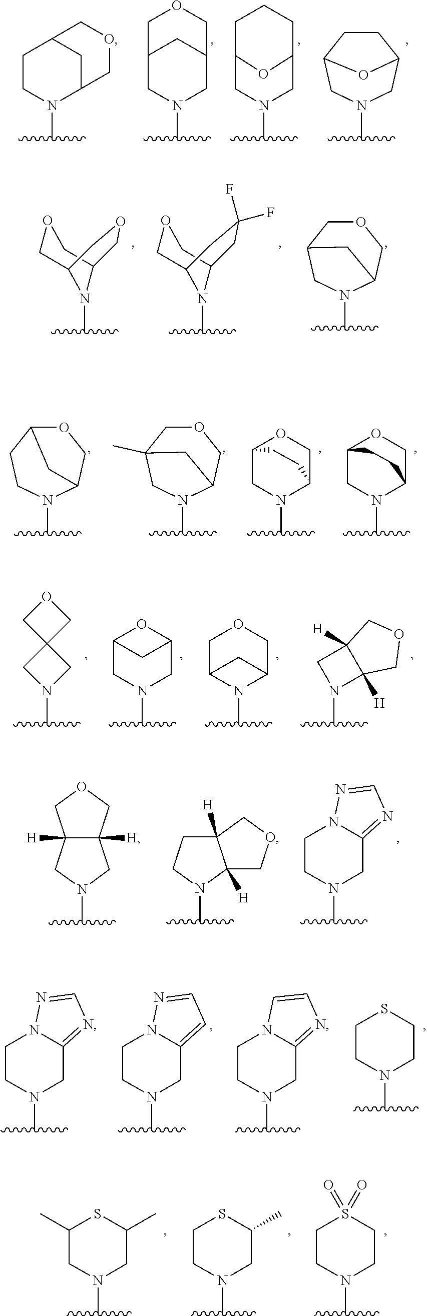

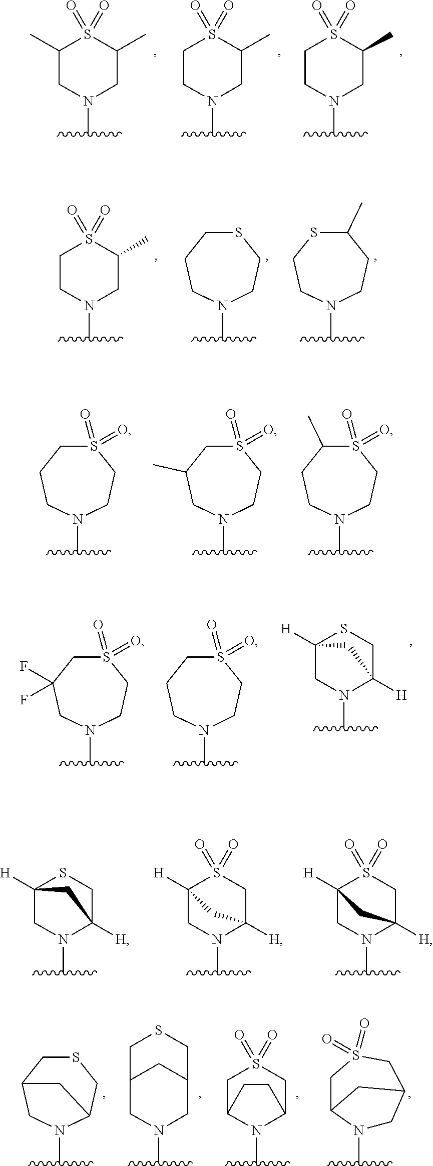

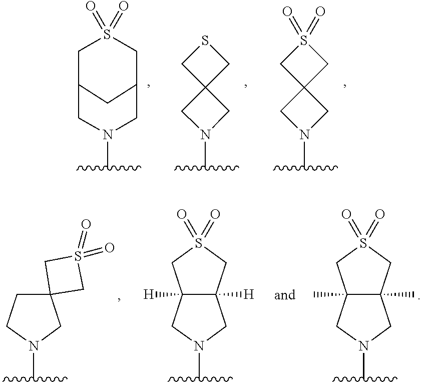

94. The compound of any one of claims 1 to 73, wherein R^{e1} is selected from the group consisting of:

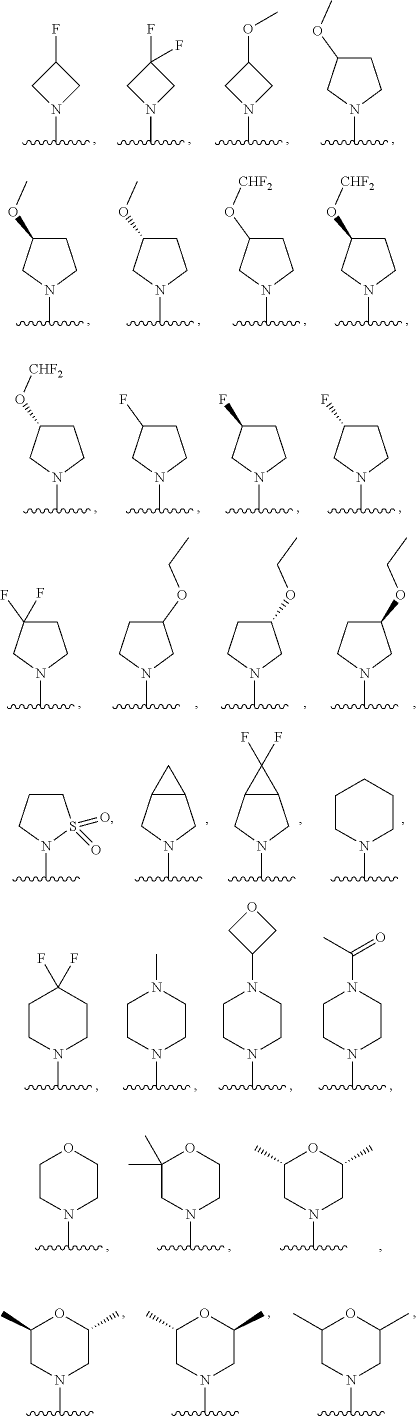

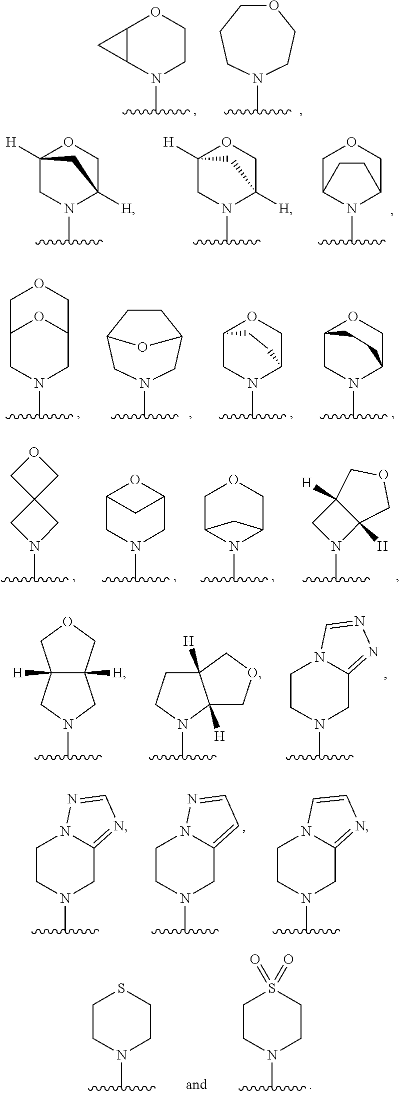

95. The compound ofany one of claims 1 to 73, wherein R^{e1} is selected from the group consisting of:

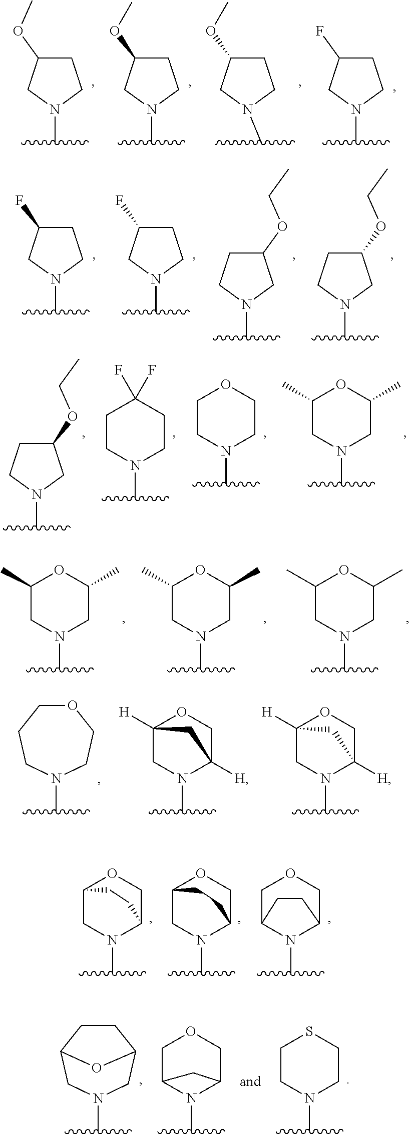

96. The compound of any one of claims 81 to 95, wherein R^{2c} is

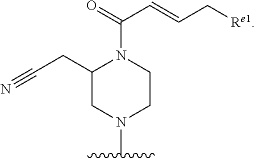

97. The compound of any one of claims 1 to 72, 74 to 79 and 81 to 95 wherein R^{e} is R^{e2}.

98. The compound of any one of claims 1 to 72, 74 to 79, 81 to 95 and 97, wherein R^{e2} is a 5-6 membered heteroaryl group containing at least one nitrogen atom, wherein the attachment point for the heteroaryl group is a carbon atom group and wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

99. The compound of any one of claims 1 to 72, 74 to 79, 81 to 95 and 97, wherein R^{e2} is selected from the group consisting of pyrimidinyl, pyrazinyl, oxazolyl, 1,2,4-oxadiazolyl, 1,3,4-oxadiazolyl, 1H-1,2,4-triazolyl, imidazolyl, 4H-1,2,4-triazolyl, 1H-1,2,4-triazolyl, 2-H-tetrazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

100. The compound of any one of claims 1 to 72, 74 to 79, 81 to 95 and 97, wherein R^{e2} is selected from the group consisting of pyrimidinyl, pyrazinyl, oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

101. The compound of any one of claims 1 to 72, 74 to 79, 81 to 95 and 97, wherein R^{e2} is selected from the group consisting of pyrimidinyl and 1,2,4-oxadiazolyl each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

102. The compound of any one of claims 1 to 72, 74 to 79, 81 to 95 and 97, wherein R^{e2} is selected from the group consisting of 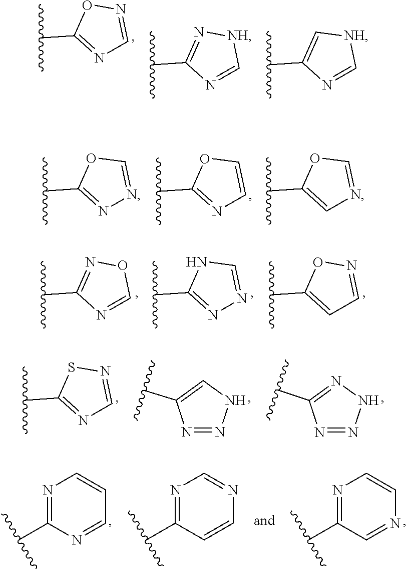
each substituted with 0, 1 or 2 substituents independently selected from halo, hydroxy, C₁-C₄ acyclic alkyl, C₁-C₄ hydroxyalkyl, C₁-C₆ alkoxy, C₁-C₄ haloalkyl, C₁-C₄ haloalkoxy, C₃-C₆ heterocyclyl and C₃-C₆ cycloalkyl optionally substituted with one or two instances of halo or methyl.

103. The compound of any one of claims 1 to 72, 74 to 79, 81 to 95 and 97, wherein R^{e2} is selected from the group consisting of 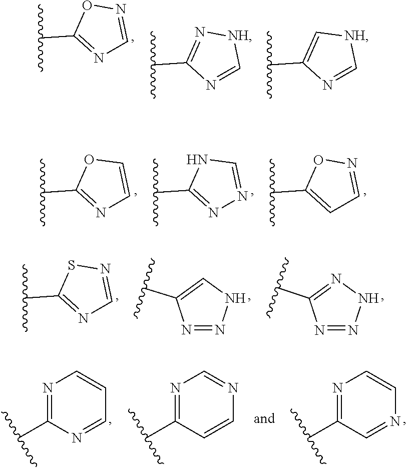
each substituted with 0, 1 or 2 substituents independently selected from —F, -Me, -Et, -ⁱPr, -tBu, —C(OH)(CH₃)₂, —CHF₂, —CF₂CH₃, —CH(F)CH₃, —CF₃, oxetanyl, cyclopropyl, 1-Me-cyclopropyl and 2-F-cyclopropyl.

104. The compound of any one of claims 1 to 72, 74 to 79, 81 to 95 and 97, wherein R^{e2} is selected from the group consisting of 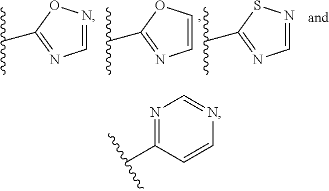
each substituted with 0, 1 or 2 substituents independently selected from —F, -Me, -Et, -ⁱPr, -tBu, —C(OH)(CH₃)₂, —CHF₂, —CF₂CH₃, —CH(F)CH₃, —CF₃, oxetanyl, cyclopropyl, 1-Me-cyclopropyl and 2-F-cyclopropyl.

105. The compound of any one of claims 1 to 72, 74 to 79, 81 to 95 and 97, wherein R^{e2} is selected from the group consisting of 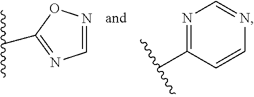 [SMILES: *c1ncno1 ; *c1ccncn1]
each substituted with 0, 1 or 2 substituents independently selected from —F, -Me, -Et, -ⁱPr, -tBu, —C(OH)(CH₃)₂, —CHF₂, —CF₂CH₃, —CH(F)CH₃, —CF₃, oxetanyl, cyclopropyl, 1-Me-cyclopropyl and 2-F-cyclopropyl.

106. The compound of any one of claims 1 to 72, 74 to 79, 81 to 95 and 97, wherein R^{e2} is selected from the group consisting of:

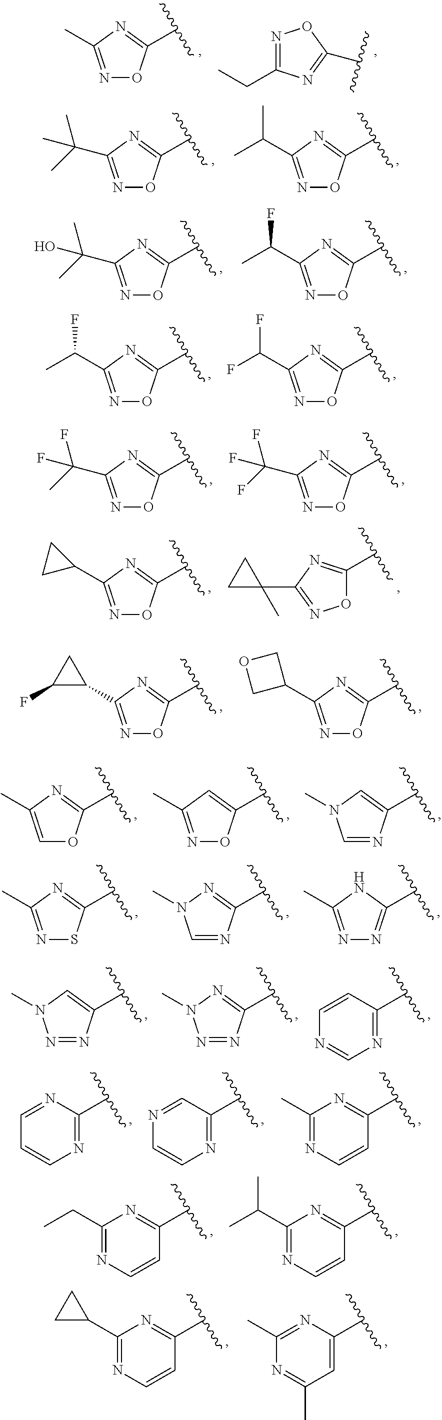

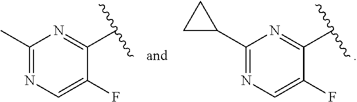

107. The compound of any one of claims 1 to 72, 74 to 79, 81 to 95 and 97, wherein R^{e2} is selected from the group consisting of:

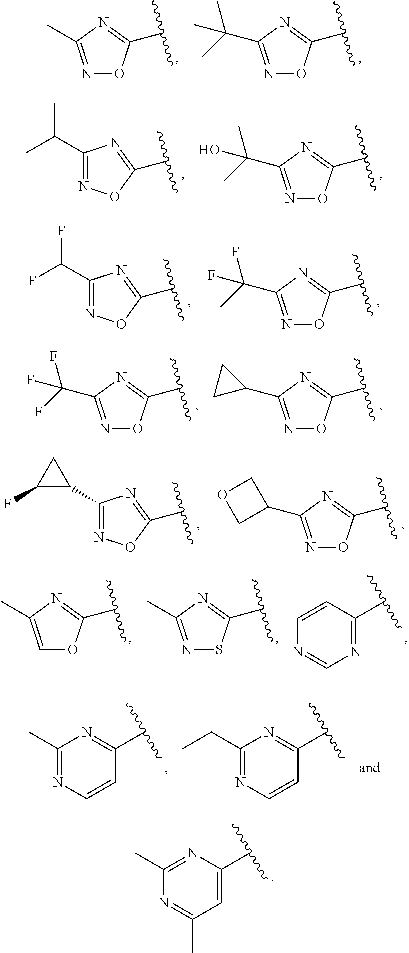

108. The compound of any one of claims 97 to 107, wherein R^{2c} is

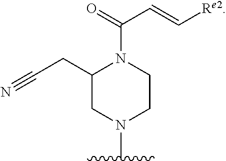

109. The compound of any one of claims 1 to 72, 74 to 79, 81 to 95 and 98-107, wherein R^{e} is R^{e3}.

110. The compound of any one of claims 1 to 72, 74 to 79, 81 to 95, 98 to 107 and 109, wherein
R^{e3} is selected from

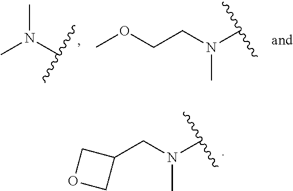

111. The compound of claim 109 or 110 wherein R^{2c} is

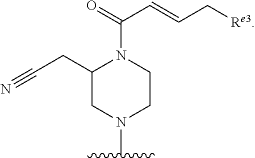

112. The compound of any one of claims 1 to 111, wherein the stereochemistry of the methylene nitrile group of R^{2c} is (S).

113. The compound of any one of claims 1 to 112, wherein R¹ is

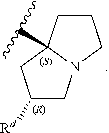

114. The compound of any one of claims 1 to 113, wherein the compound is selected from the group consisting of: 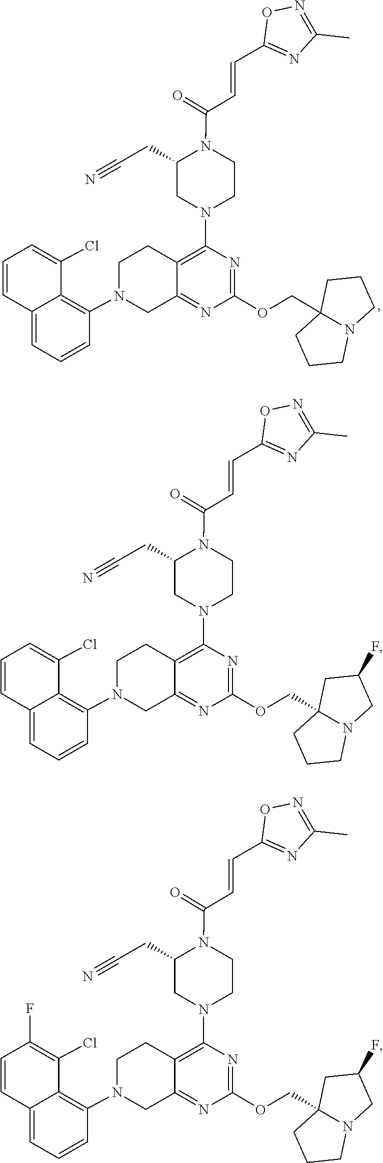 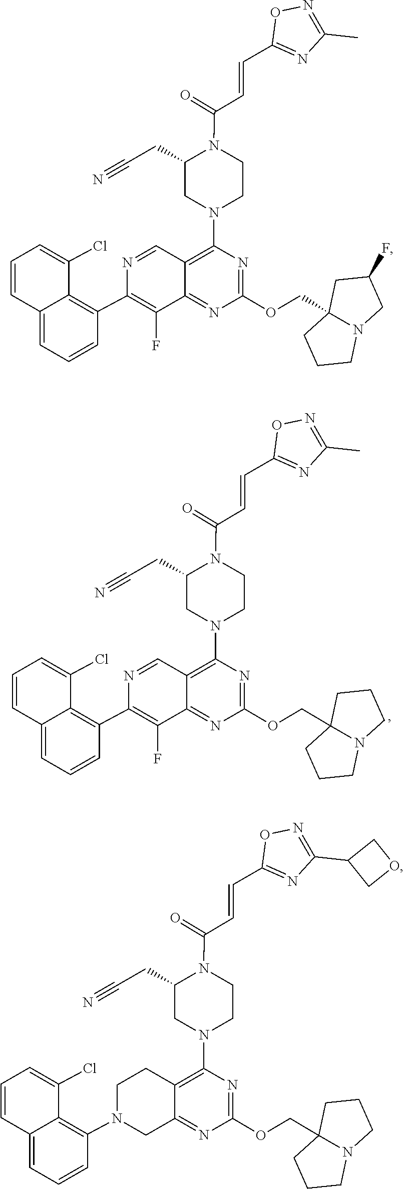 [SMILES: Cc1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; Cc1noc(/C=C/C(=O)N2CCN(c3nc(OCC45CCCN4CCC5)nc4c(F)c(-c5cccc6cccc(Cl)c56)ncc34)C[C@@H]2CC#N)n1 ; N#CC[C@H]1CN(c2nc(OCC34CCCN3CCC4)nc3c2CCN(c2cccc4cccc(Cl)c24)C3)CCN1C(=O)/C=C/c1nc(C2COC2)no1] 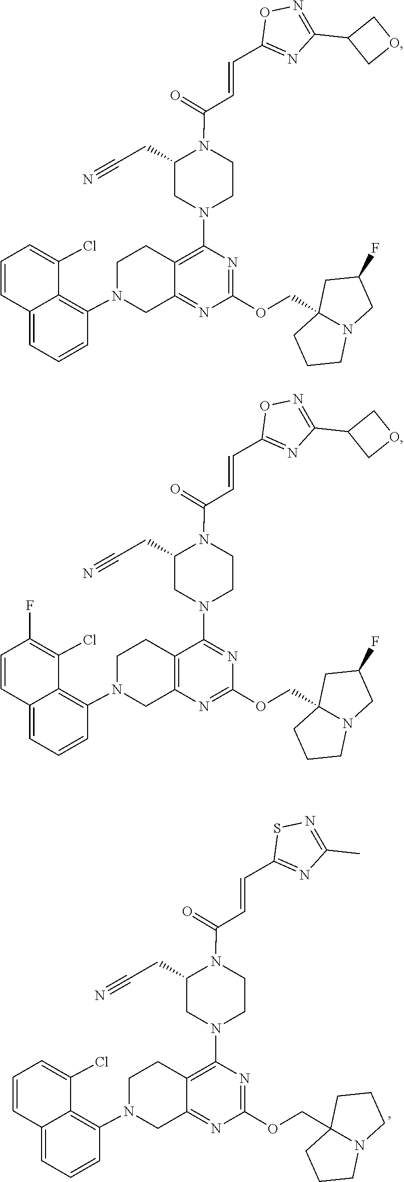 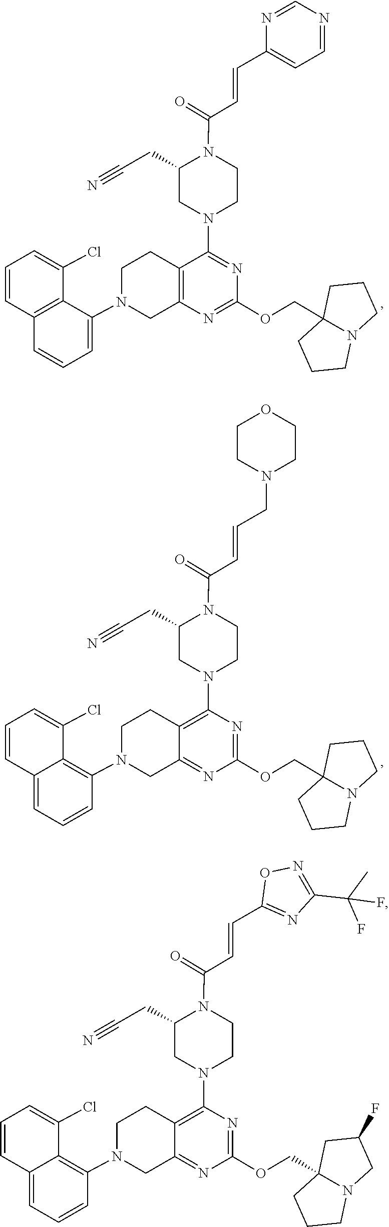 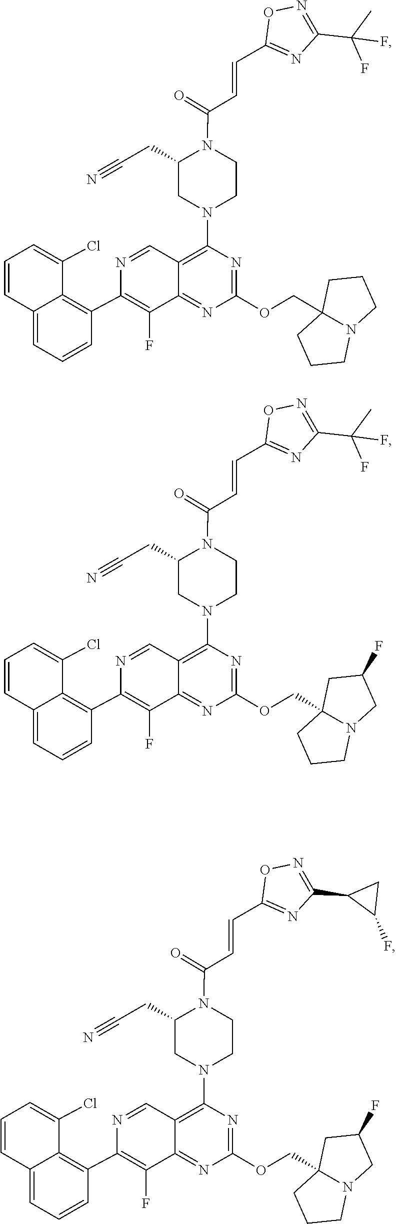 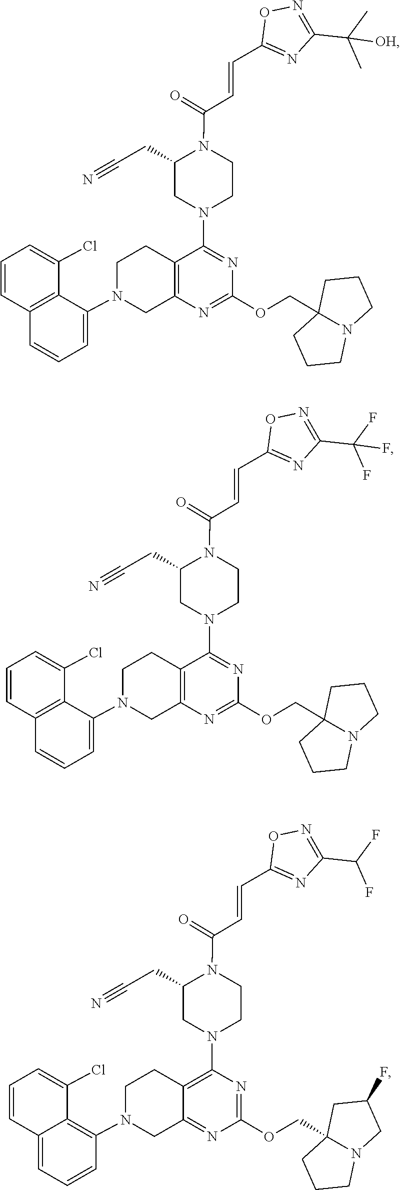 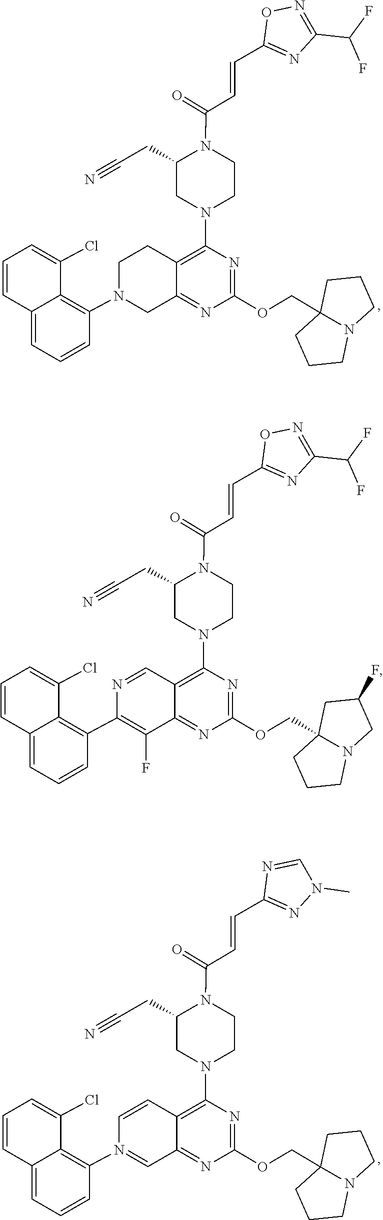 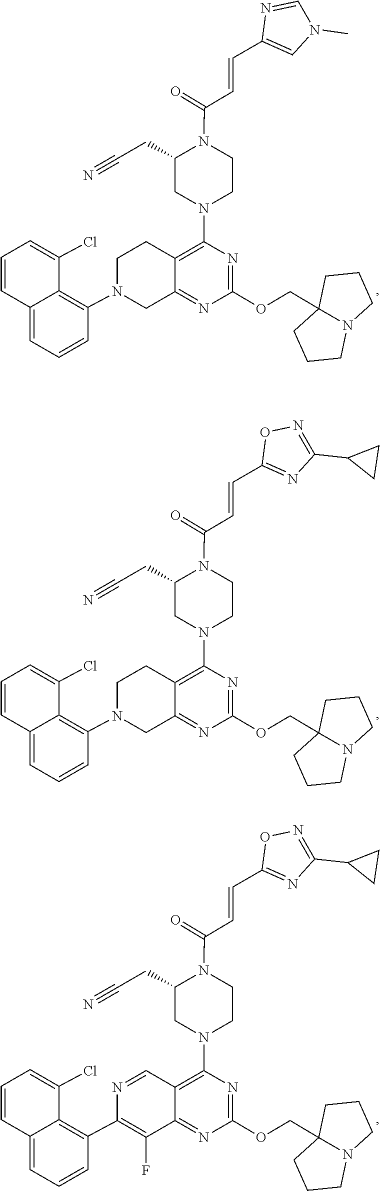 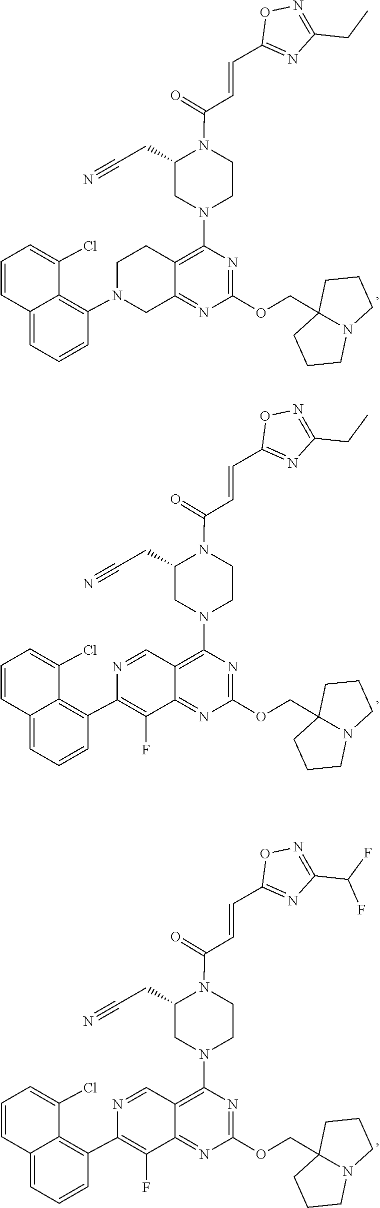 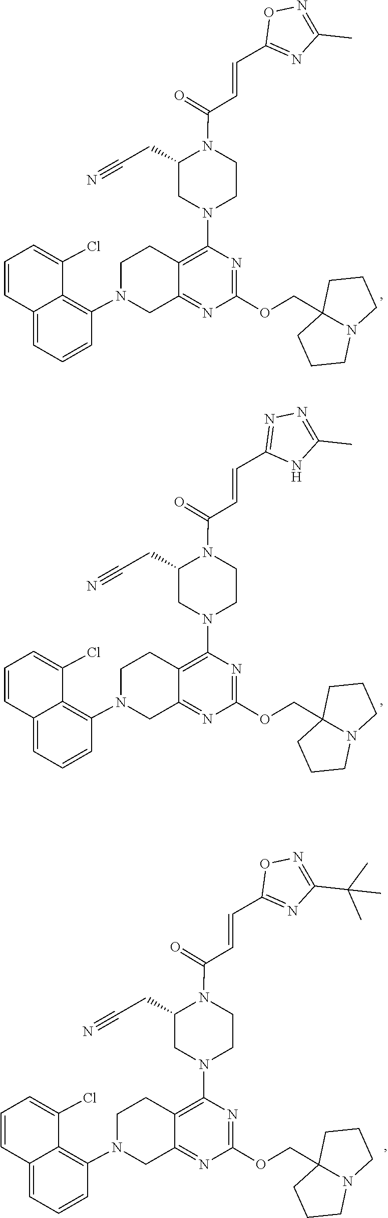 [SMILES: Cc1noc(/C=C/C(=O)N2CCN(c3nc(OCC45CCCN4CCC5)nc4c3CCN(c3cccc5cccc(Cl)c35)C4)C[C@@H]2CC#N)n1 ; Cc1nnc(/C=C/C(=O)N2CCN(c3nc(OCC45CCCN4CCC5)nc4c3CCN(c3cccc5cccc(Cl)c35)C4)C[C@@H]2CC#N)[nH]1 ; CC(C)(C)c1noc(/C=C/C(=O)N2CCN(c3nc(OCC45CCCN4CCC5)nc4c3CCN(c3cccc5cccc(Cl)c35)C4)C[C@@H]2CC#N)n1] 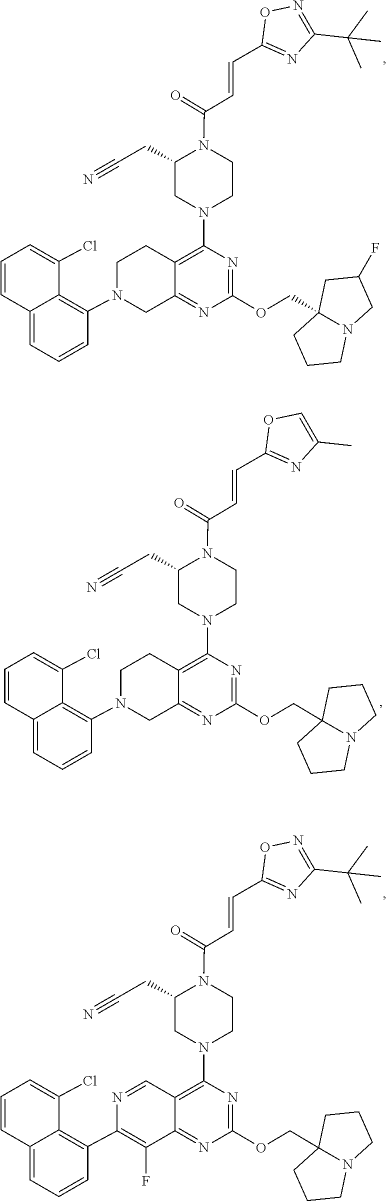 [SMILES: CC(C)(C)c1noc(/C=C/C(=O)N2CCN(c3nc(OC[C@@]45CCCN4CC(F)C5)nc4c3CCN(c3cccc5cccc(Cl)c35)C4)C[C@@H]2CC#N)n1 ; Cc1coc(/C=C/C(=O)N2CCN(c3nc(OCC45CCCN4CCC5)nc4c3CCN(c3cccc5cccc(Cl)c35)C4)C[C@@H]2CC#N)n1 ; CC(C)(C)c1noc(/C=C/C(=O)N2CCN(c3nc(OCC45CCCN4CCC5)nc4c(F)c(-c5cccc6cccc(Cl)c56)ncc34)C[C@@H]2CC#N)n1] 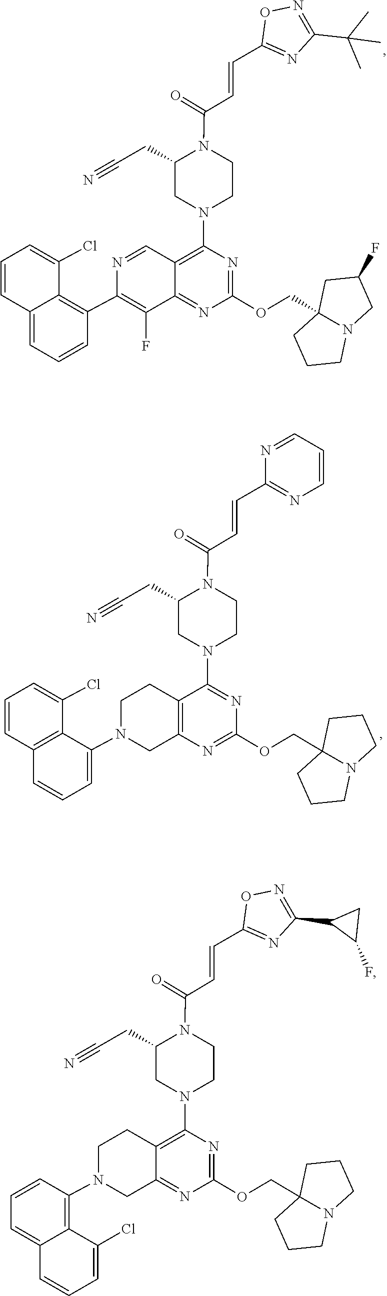 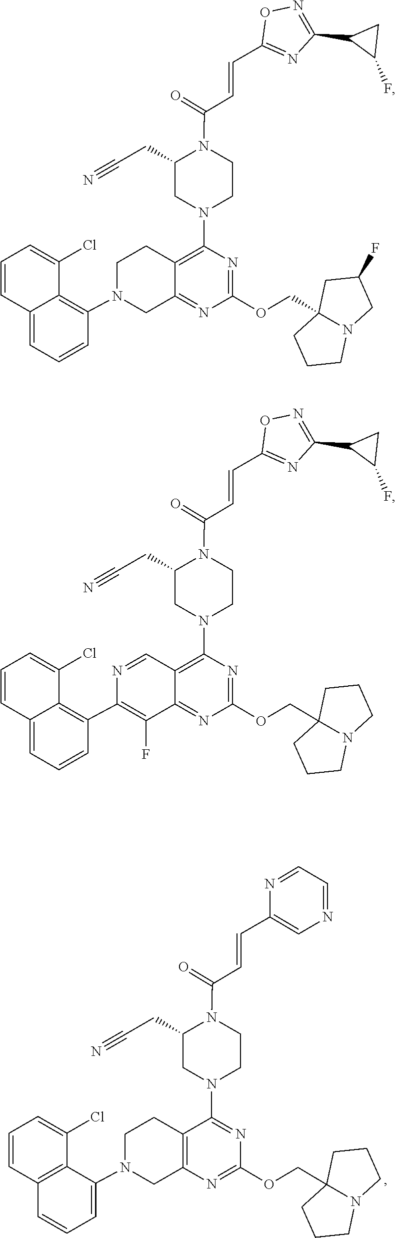 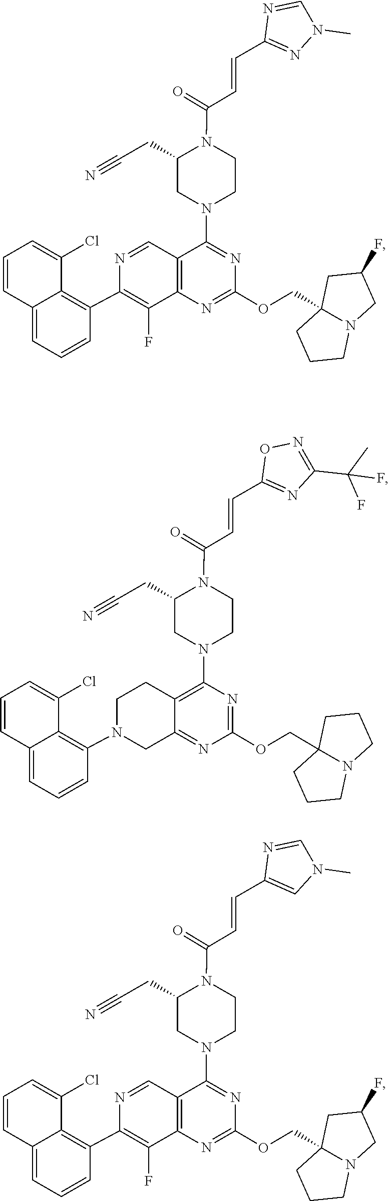 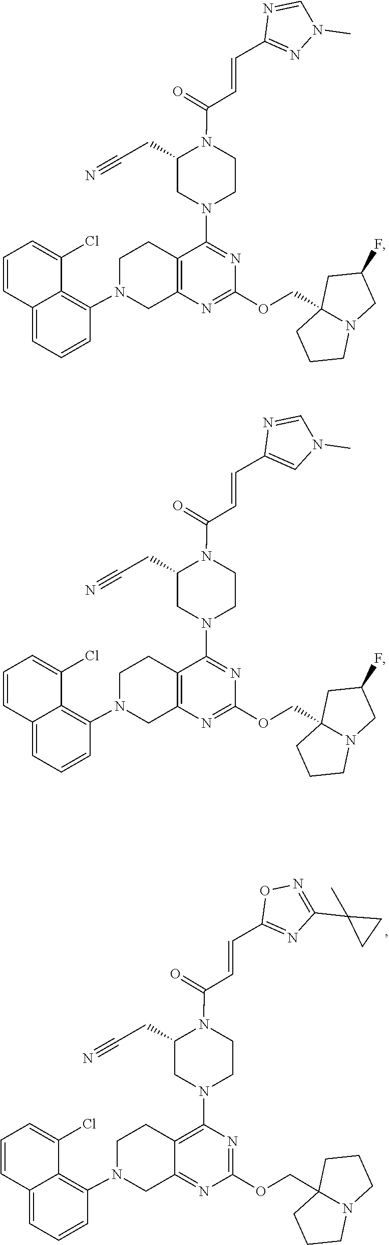 [SMILES: Cn1cnc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1 ; Cn1cnc(/C=C/C(=O)N2CCN(c3nc(OC[C@@]45CCCN4C[C@H](F)C5)nc4c3CCN(c3cccc5cccc(Cl)c35)C4)C[C@@H]2CC#N)c1 ; CC1(c2noc(/C=C/C(=O)N3CCN(c4nc(OCC56CCCN5CCC6)nc5c4CCN(c4cccc6cccc(Cl)c46)C5)C[C@@H]3CC#N)n2)CC1] 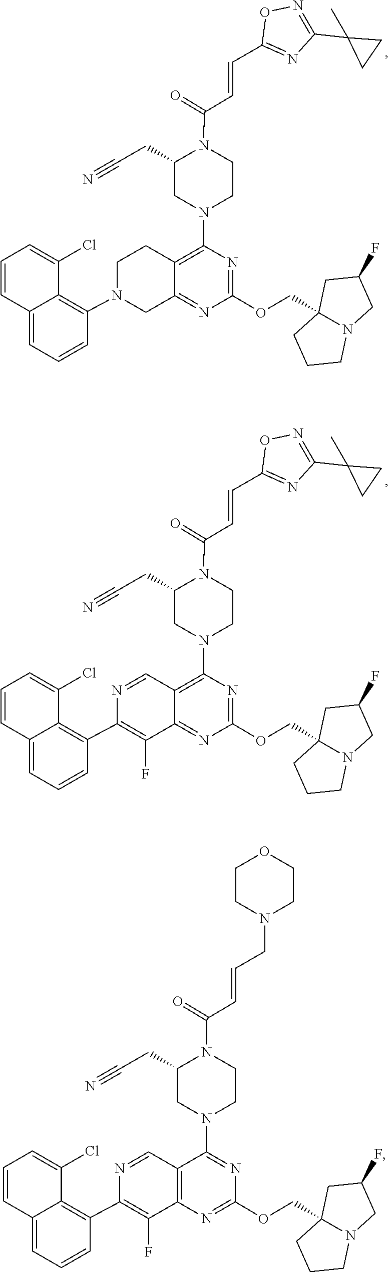 [SMILES: CC1(c2noc(/C=C/C(=O)N3CCN(c4nc(OC[C@@]56CCCN5C[C@H](F)C6)nc5c4CCN(c4cccc6cccc(Cl)c46)C5)C[C@@H]3CC#N)n2)CC1 ; CC1(c2noc(/C=C/C(=O)N3CCN(c4nc(OC[C@@]56CCCN5C[C@H](F)C6)nc5c(F)c(-c6cccc7cccc(Cl)c67)ncc45)C[C@@H]3CC#N)n2)CC1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCOCC1] 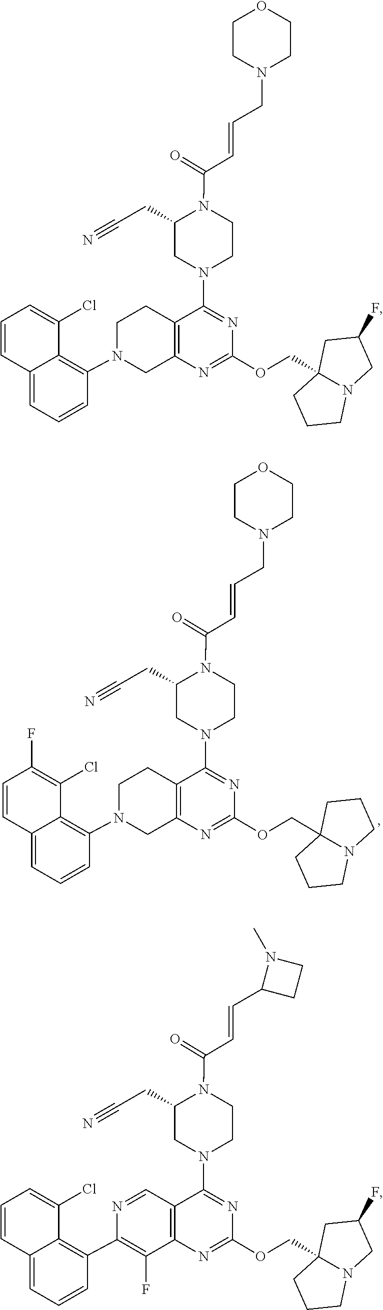 [SMILES: N#CC[C@H]1CN(c2nc(OC[C@@]34CCCN3C[C@H](F)C4)nc3c2CCN(c2cccc4cccc(Cl)c24)C3)CCN1C(=O)/C=C/CN1CCOCC1 ; N#CC[C@H]1CN(c2nc(OCC34CCCN3CCC4)nc3c2CCN(c2cccc4ccc(F)c(Cl)c24)C3)CCN1C(=O)/C=C/CN1CCOCC1 ; CN1CCC1/C=C/C(=O)N1CCN(c2nc(OC[C@@]34CCCN3C[C@H](F)C4)nc3c(F)c(-c4cccc5cccc(Cl)c45)ncc23)C[C@@H]1CC#N] 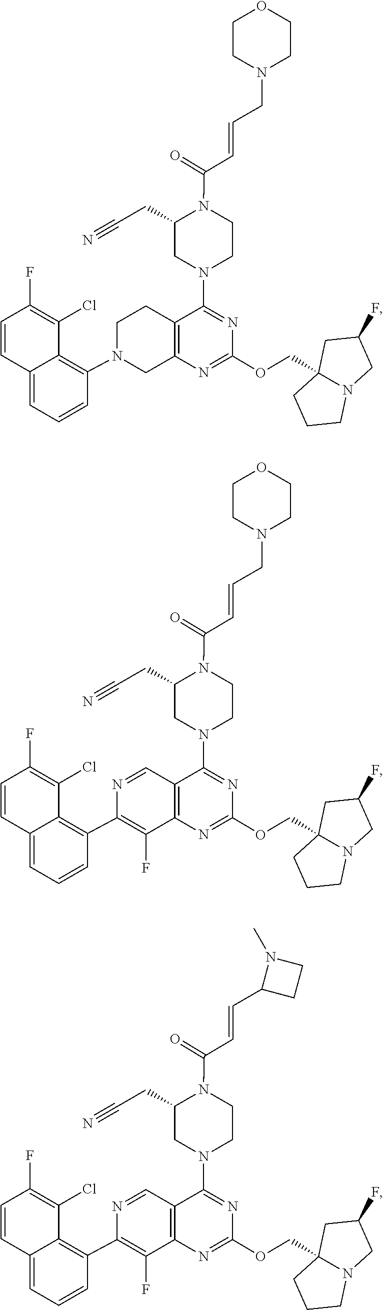 [SMILES: N#CC[C@H]1CN(c2nc(OC[C@@]34CCCN3C[C@H](F)C4)nc3c2CCN(c2cccc4ccc(F)c(Cl)c24)C3)CCN1C(=O)/C=C/CN1CCOCC1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCOCC1 ; CN1CCC1/C=C/C(=O)N1CCN(c2nc(OC[C@@]34CCCN3C[C@H](F)C4)nc3c(F)c(-c4cccc5ccc(F)c(Cl)c45)ncc23)C[C@@H]1CC#N] 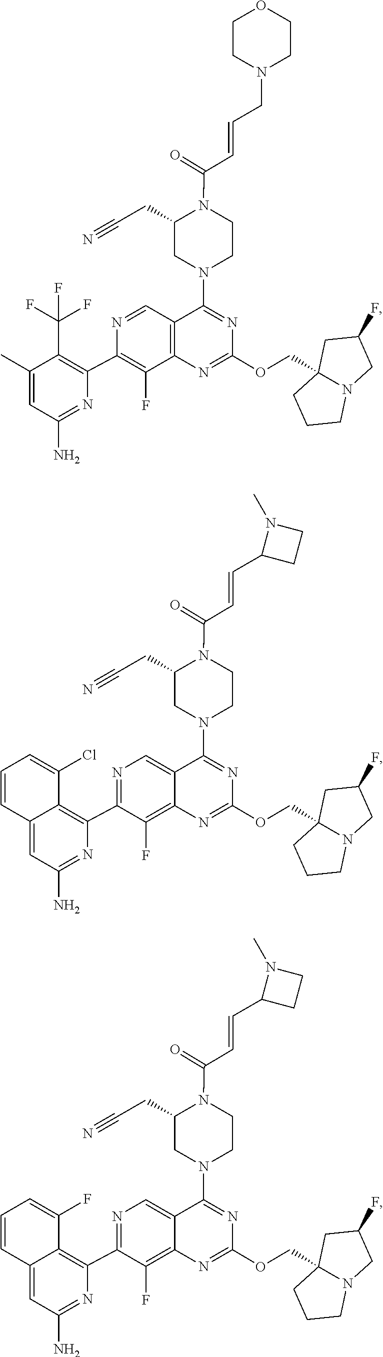 [SMILES: Cc1cc(N)nc(-c2ncc3c(N4CCN(C(=O)/C=C/CN5CCOCC5)[C@@H](CC#N)C4)nc(OC[C@@]45CCCN4C[C@H](F)C5)nc3c2F)c1C(F)(F)F ; CN1CCC1/C=C/C(=O)N1CCN(c2nc(OC[C@@]34CCCN3C[C@H](F)C4)nc3c(F)c(-c4nc(N)cc5cccc(Cl)c45)ncc23)C[C@@H]1CC#N ; CN1CCC1/C=C/C(=O)N1CCN(c2nc(OC[C@@]34CCCN3C[C@H](F)C4)nc3c(F)c(-c4nc(N)cc5cccc(F)c45)ncc23)C[C@@H]1CC#N] 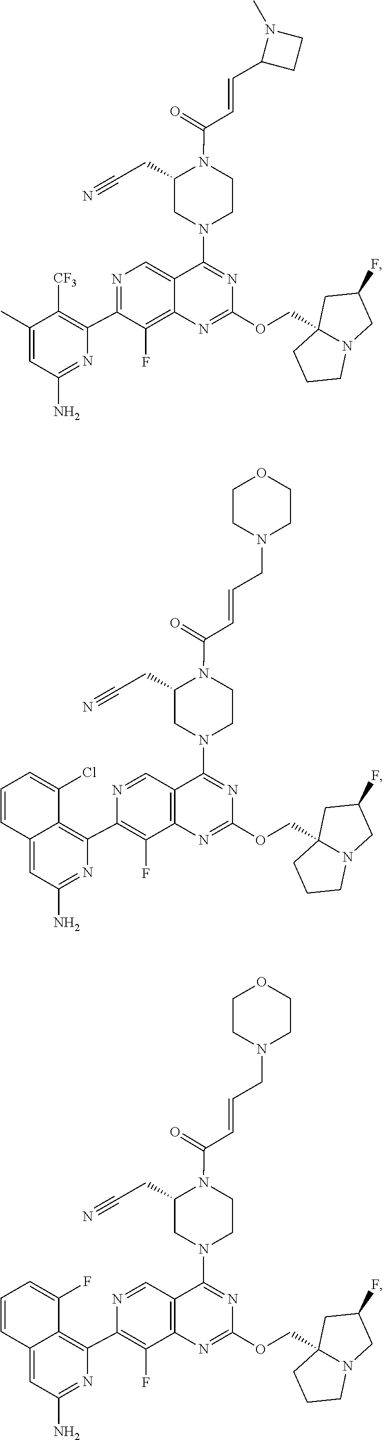 [SMILES: Cc1cc(N)nc(-c2ncc3c(N4CCN(C(=O)/C=C/C5CCN5C)[C@@H](CC#N)C4)nc(OC[C@@]45CCCN4C[C@H](F)C5)nc3c2F)c1C(F)(F)F ; N#CC[C@H]1CN(c2nc(OC[C@@]34CCCN3C[C@H](F)C4)nc3c(F)c(-c4nc(N)cc5cccc(Cl)c45)ncc23)CCN1C(=O)/C=C/CN1CCOCC1 ; N#CC[C@H]1CN(c2nc(OC[C@@]34CCCN3C[C@H](F)C4)nc3c(F)c(-c4nc(N)cc5cccc(F)c45)ncc23)CCN1C(=O)/C=C/CN1CCOCC1] 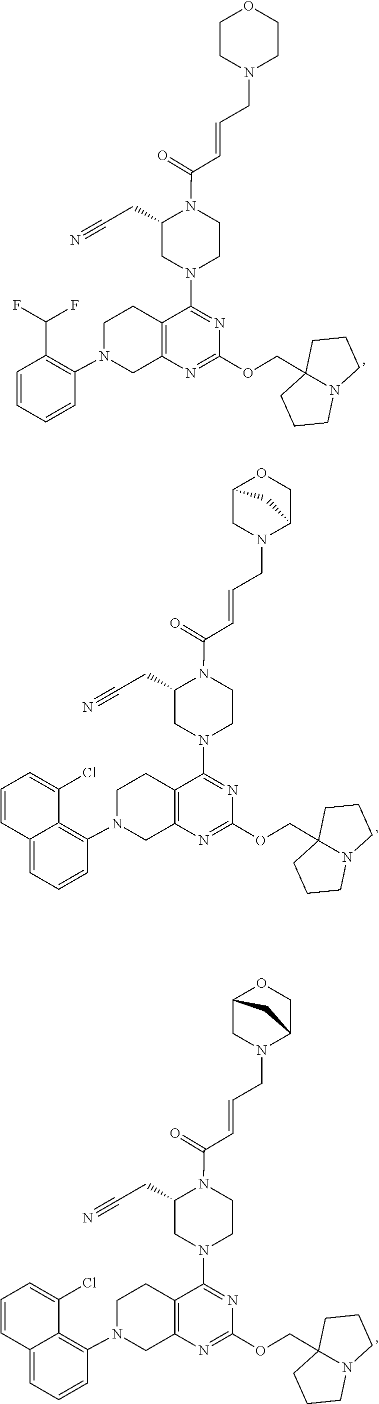 [SMILES: N#CC[C@H]1CN(c2nc(OCC34CCCN3CCC4)nc3c2CCN(c2ccccc2C(F)F)C3)CCN1C(=O)/C=C/CN1CCOCC1 ; N#CC[C@H]1CN(c2nc(OCC34CCCN3CCC4)nc3c2CCN(c2cccc4cccc(Cl)c24)C3)CCN1C(=O)/C=C/CN1C[C@@H]2C[C@H]1CO2 ; N#CC[C@H]1CN(c2nc(OCC34CCCN3CCC4)nc3c2CCN(c2cccc4cccc(Cl)c24)C3)CCN1C(=O)/C=C/CN1C[C@H]2C[C@@H]1CO2] 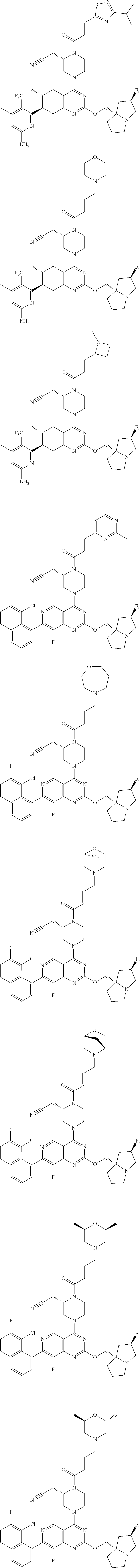 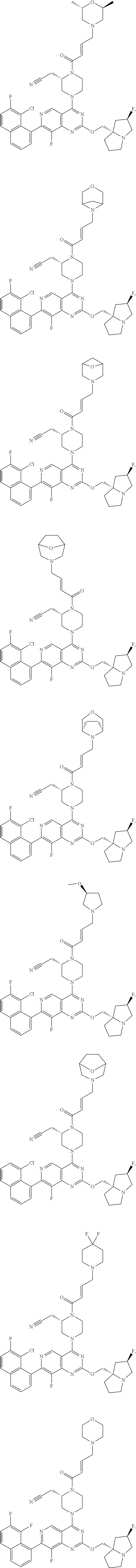 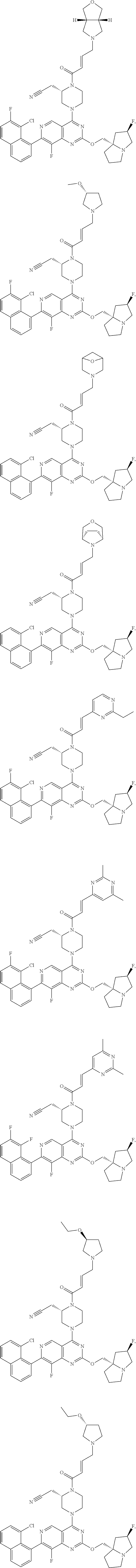 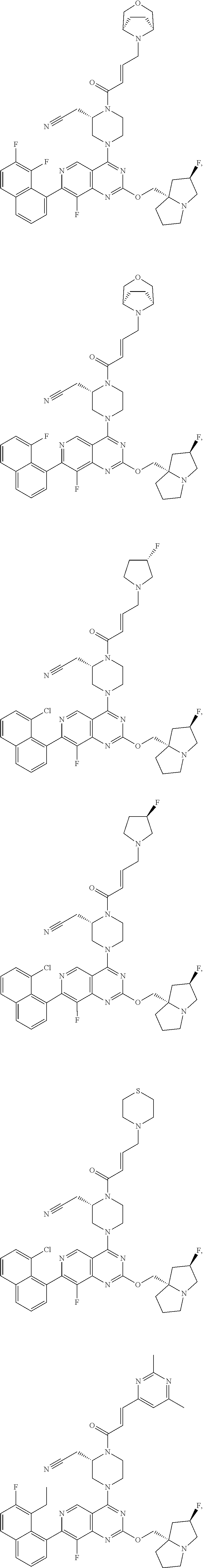 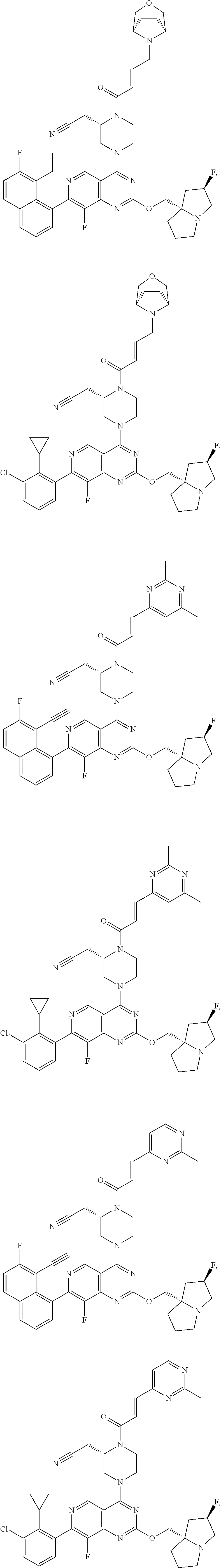 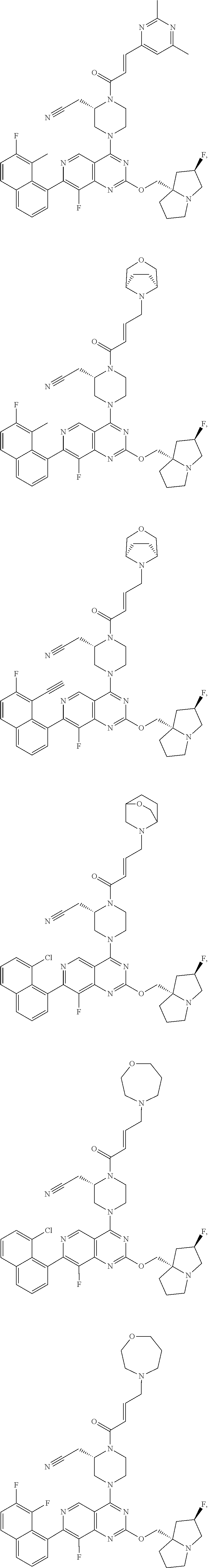 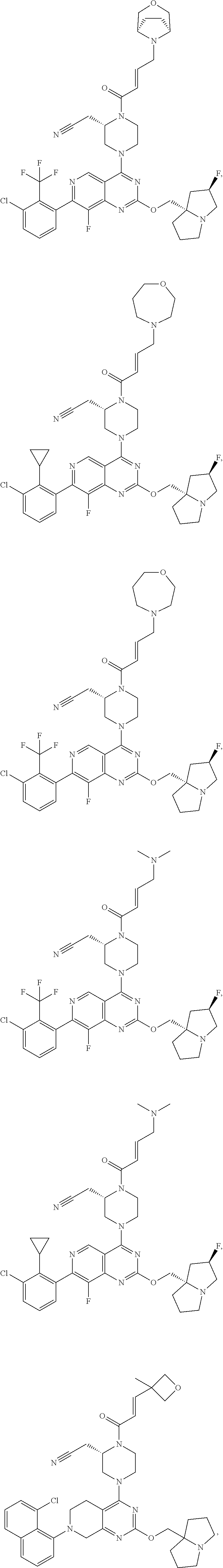 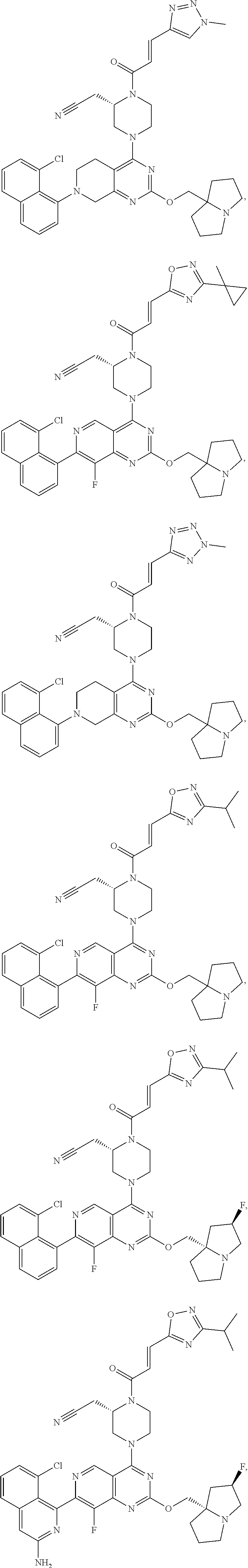 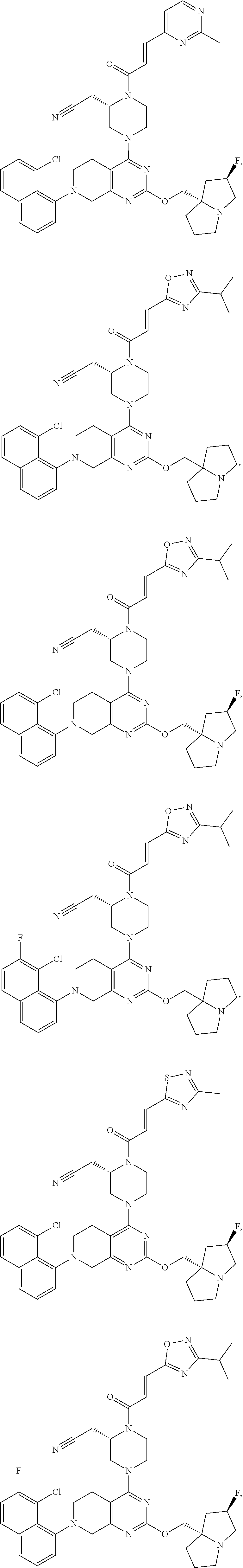 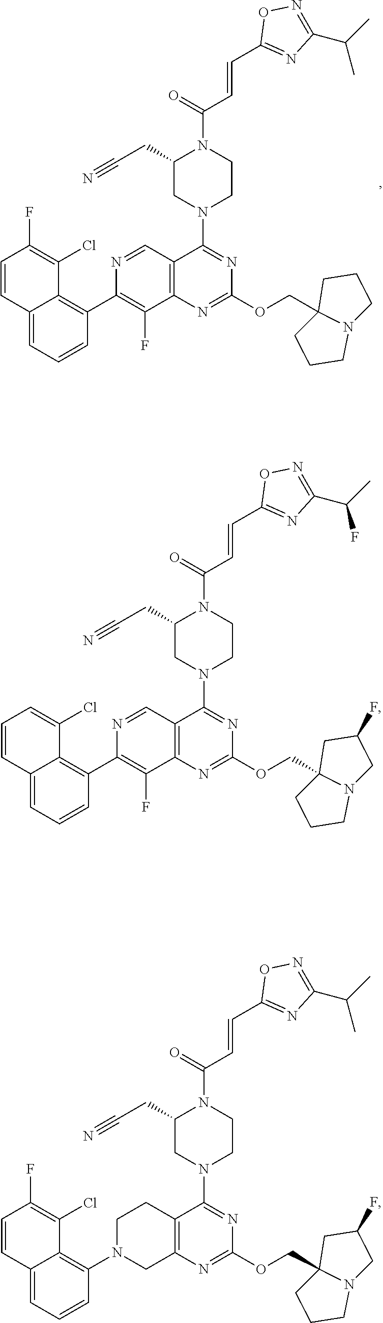 [SMILES: CC(C)c1noc(/C=C/C(=O)N2CCN(c3nc(OCC45CCCN4CCC5)nc4c(F)c(-c5cccc6ccc(F)c(Cl)c56)ncc34)C[C@@H]2CC#N)n1 ; C[C@@H](F)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; CC(C)c1noc(/C=C/C(=O)N2CCN(c3nc(OC[C@]45CCCN4C[C@H](F)C5)nc4c3CCN(c3cccc5ccc(F)c(Cl)c35)C4)C[C@@H]2CC#N)n1] 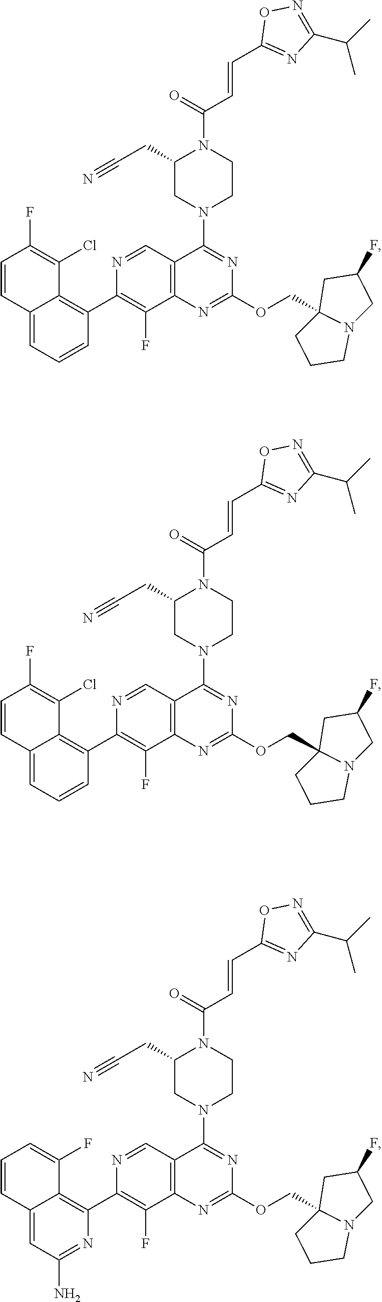 [SMILES: CC(C)c1noc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1 ; CC(C)c1noc(/C=C/C(=O)N2CCN(c3nc(OC[C@]45CCCN4C[C@H](F)C5)nc4c(F)c(-c5cccc6ccc(F)c(Cl)c56)ncc34)C[C@@H]2CC#N)n1 ; CC(C)c1noc(/C=C/C(=O)N2CCN(c3nc(OC[C@@]45CCCN4C[C@H](F)C5)nc4c(F)c(-c5nc(N)cc6cccc(F)c56)ncc34)C[C@@H]2CC#N)n1] 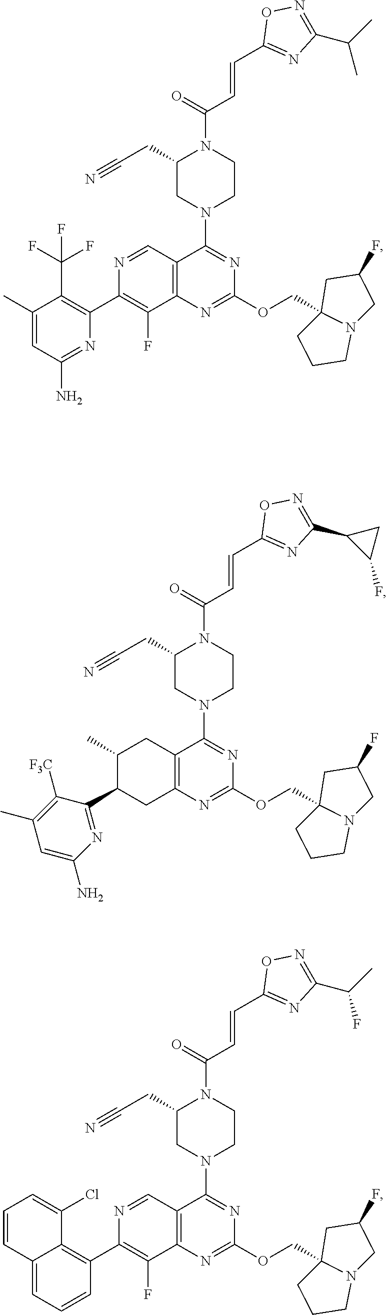 [SMILES: Cc1cc(N)nc(-c2ncc3c(N4CCN(C(=O)/C=C/c5nc(C(C)C)no5)[C@@H](CC#N)C4)nc(OC[C@@]45CCCN4C[C@H](F)C5)nc3c2F)c1C(F)(F)F ; Cc1cc(N)nc([C@@H]2Cc3nc(OC[C@@]45CCCN4C[C@H](F)C5)nc(N4CCN(C(=O)/C=C/c5nc([C@H]6C[C@@H]6F)no5)[C@@H](CC#N)C4)c3C[C@H]2C)c1C(F)(F)F ; C[C@H](F)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1] 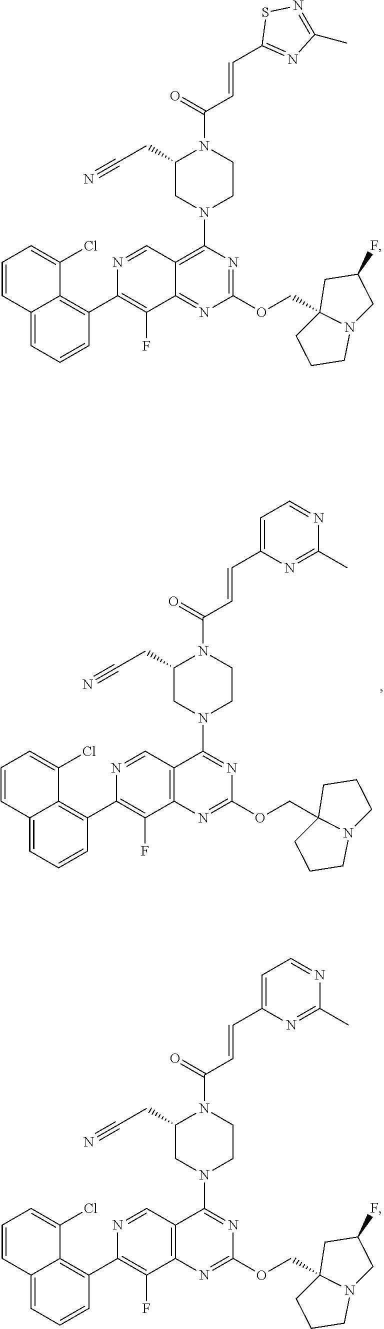 [SMILES: Cc1nsc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; Cc1nccc(/C=C/C(=O)N2CCN(c3nc(OCC45CCCN4CCC5)nc4c(F)c(-c5cccc6cccc(Cl)c56)ncc34)C[C@@H]2CC#N)n1 ; Cc1nccc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1] 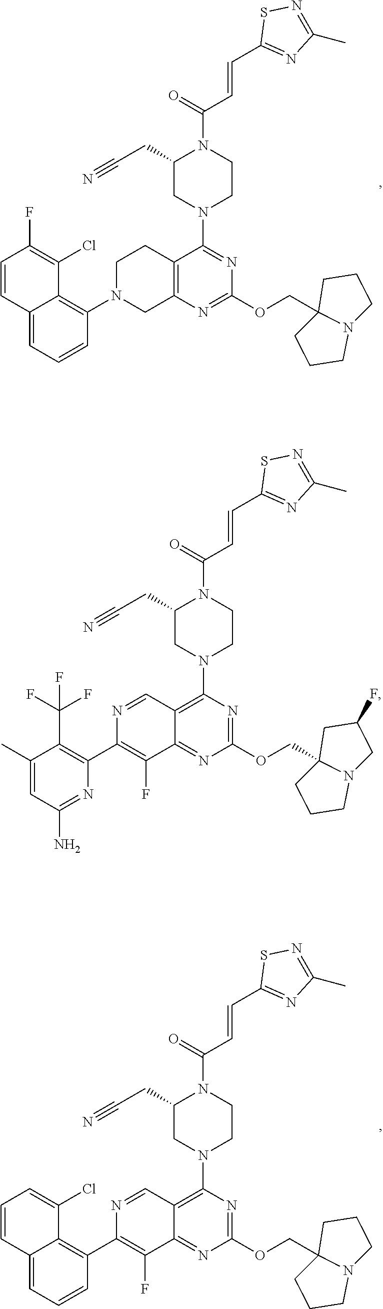 [SMILES: Cc1nsc(/C=C/C(=O)N2CCN(c3nc(OCC45CCCN4CCC5)nc4c3CCN(c3cccc5ccc(F)c(Cl)c35)C4)C[C@@H]2CC#N)n1 ; Cc1nsc(/C=C/C(=O)N2CCN(c3nc(OC[C@@]45CCCN4C[C@H](F)C5)nc4c(F)c(-c5nc(N)cc(C)c5C(F)(F)F)ncc34)C[C@@H]2CC#N)n1 ; Cc1nsc(/C=C/C(=O)N2CCN(c3nc(OCC45CCCN4CCC5)nc4c(F)c(-c5cccc6cccc(Cl)c56)ncc34)C[C@@H]2CC#N)n1] 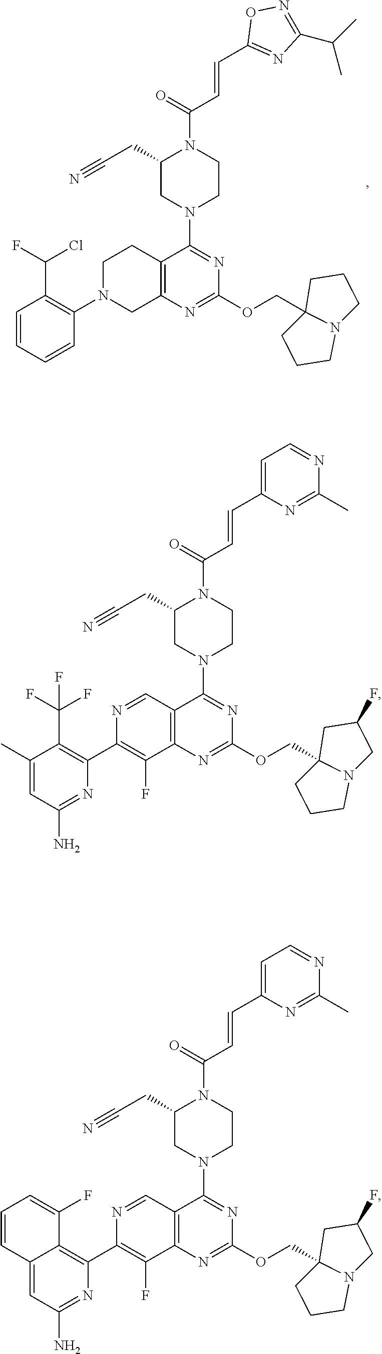 [SMILES: CC(C)c1noc(/C=C/C(=O)N2CCN(c3nc(OCC45CCCN4CCC5)nc4c3CCN(c3ccccc3C(F)Cl)C4)C[C@@H]2CC#N)n1 ; Cc1nccc(/C=C/C(=O)N2CCN(c3nc(OC[C@@]45CCCN4C[C@H](F)C5)nc4c(F)c(-c5nc(N)cc(C)c5C(F)(F)F)ncc34)C[C@@H]2CC#N)n1 ; Cc1nccc(/C=C/C(=O)N2CCN(c3nc(OC[C@@]45CCCN4C[C@H](F)C5)nc4c(F)c(-c5nc(N)cc6cccc(F)c56)ncc34)C[C@@H]2CC#N)n1] 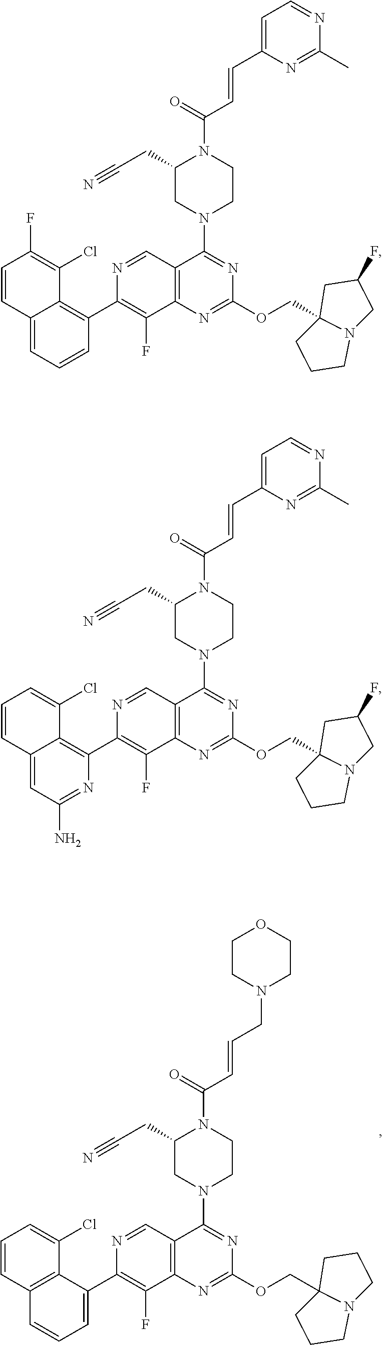 [SMILES: Cc1nccc(/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1 ; Cc1nccc(/C=C/C(=O)N2CCN(c3nc(OC[C@@]45CCCN4C[C@H](F)C5)nc4c(F)c(-c5nc(N)cc6cccc(Cl)c56)ncc34)C[C@@H]2CC#N)n1 ; N#CC[C@H]1CN(c2nc(OCC34CCCN3CCC4)nc3c(F)c(-c4cccc5cccc(Cl)c45)ncc23)CCN1C(=O)/C=C/CN1CCOCC1] 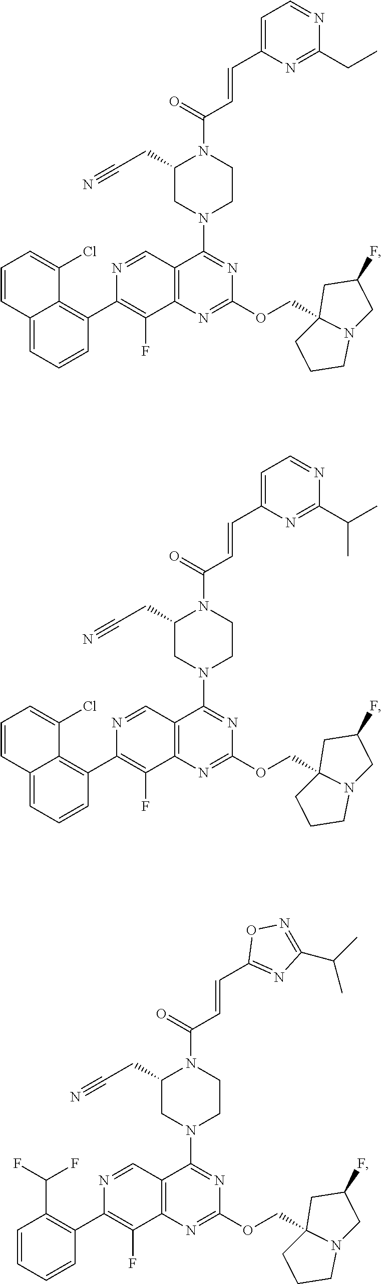 [SMILES: CCc1nccc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; CC(C)c1nccc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; CC(C)c1noc(/C=C/C(=O)N2CCN(c3nc(OC[C@@]45CCCN4C[C@H](F)C5)nc4c(F)c(-c5ccccc5C(F)F)ncc34)C[C@@H]2CC#N)n1] 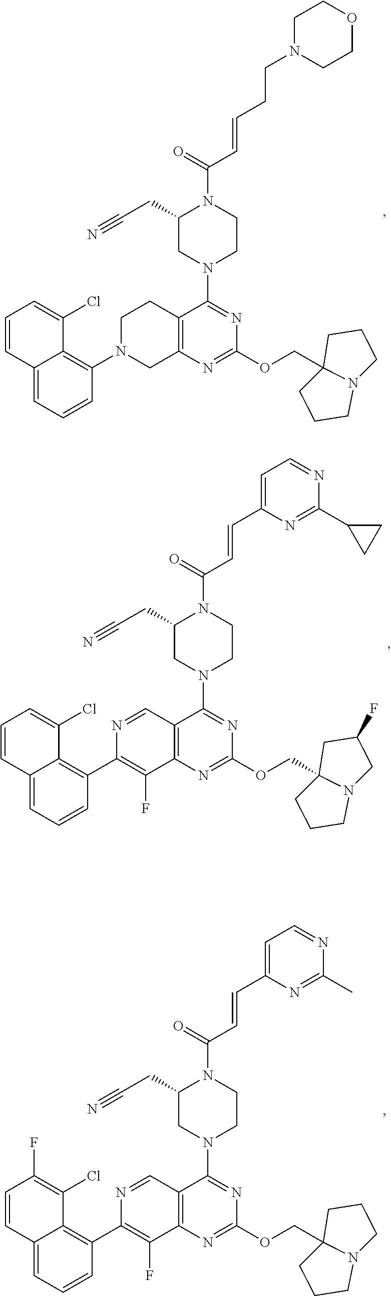 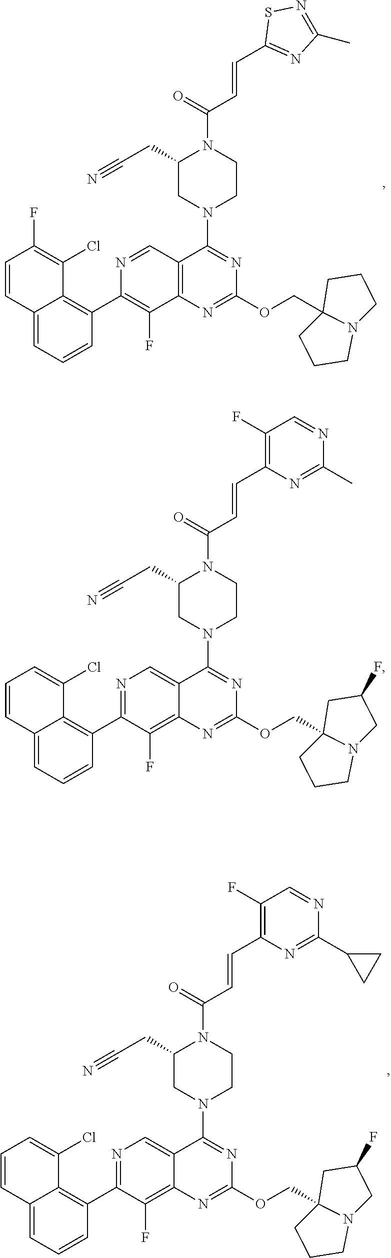 [SMILES: Cc1nsc(/C=C/C(=O)N2CCN(c3nc(OCC45CCCN4CCC5)nc4c(F)c(-c5cccc6ccc(F)c(Cl)c56)ncc34)C[C@@H]2CC#N)n1 ; Cc1ncc(F)c(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/c1nc(C2CC2)ncc1F] 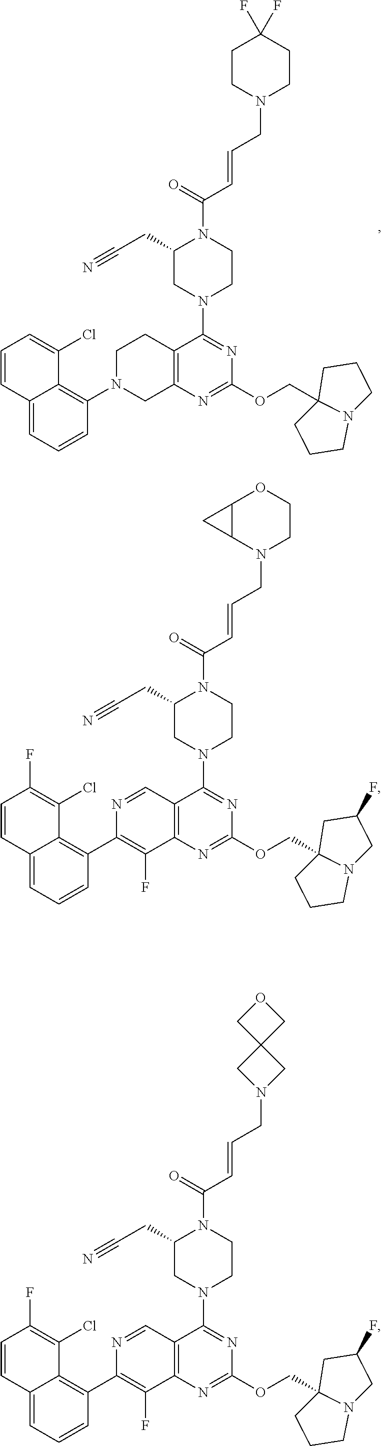 [SMILES: N#CC[C@H]1CN(c2nc(OCC34CCCN3CCC4)nc3c2CCN(c2cccc4cccc(Cl)c24)C3)CCN1C(=O)/C=C/CN1CCC(F)(F)CC1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCOC2CC21 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2(COC2)C1] 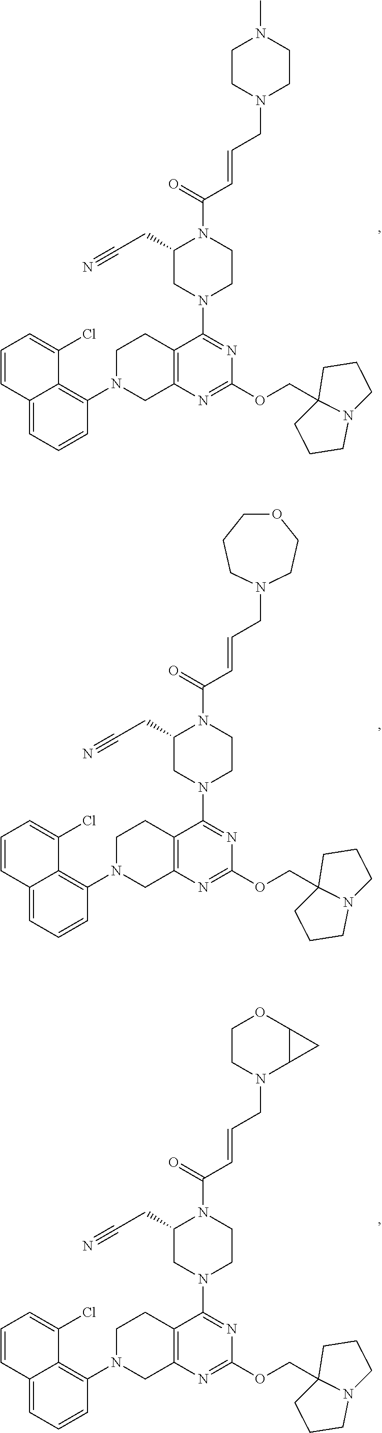 [SMILES: CN1CCN(C/C=C/C(=O)N2CCN(c3nc(OCC45CCCN4CCC5)nc4c3CCN(c3cccc5cccc(Cl)c35)C4)C[C@@H]2CC#N)CC1 ; N#CC[C@H]1CN(c2nc(OCC34CCCN3CCC4)nc3c2CCN(c2cccc4cccc(Cl)c24)C3)CCN1C(=O)/C=C/CN1CCCOCC1 ; N#CC[C@H]1CN(c2nc(OCC34CCCN3CCC4)nc3c2CCN(c2cccc4cccc(Cl)c24)C3)CCN1C(=O)/C=C/CN1CCOC2CC21] 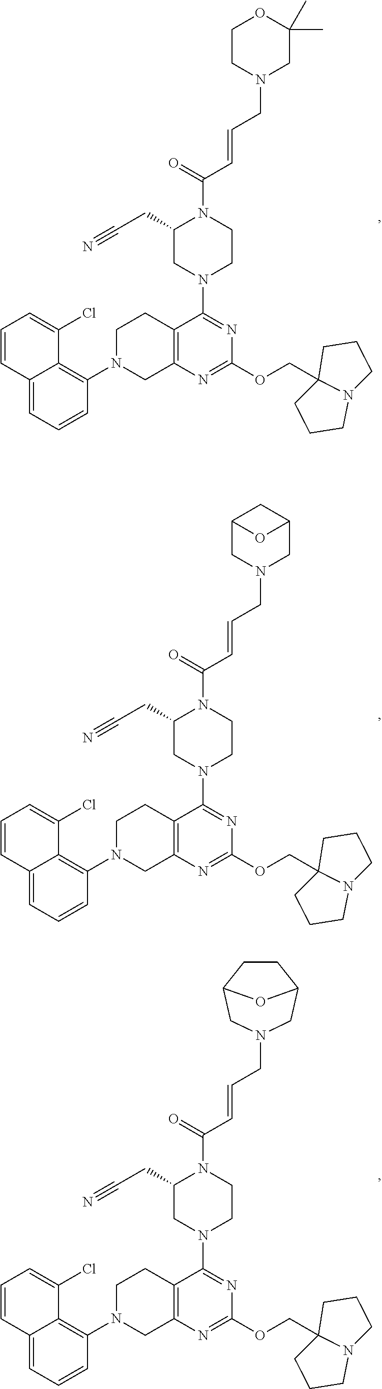 [SMILES: CC1(C)CN(C/C=C/C(=O)N2CCN(c3nc(OCC45CCCN4CCC5)nc4c3CCN(c3cccc5cccc(Cl)c35)C4)C[C@@H]2CC#N)CCO1 ; N#CC[C@H]1CN(c2nc(OCC34CCCN3CCC4)nc3c2CCN(c2cccc4cccc(Cl)c24)C3)CCN1C(=O)/C=C/CN1CC2CC(C1)O2 ; N#CC[C@H]1CN(c2nc(OCC34CCCN3CCC4)nc3c2CCN(c2cccc4cccc(Cl)c24)C3)CCN1C(=O)/C=C/CN1CC2CCC(C1)O2] 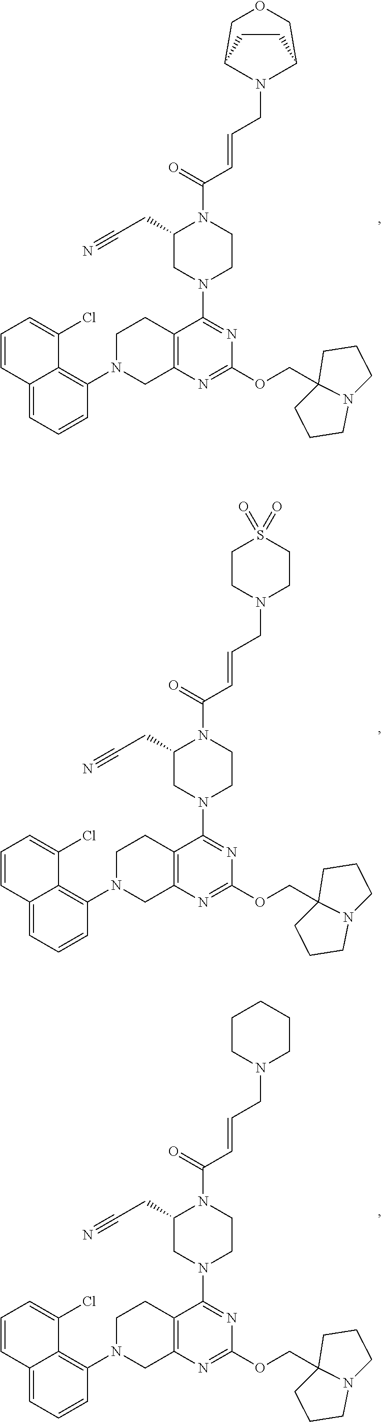 [SMILES: N#CC[C@H]1CN(c2nc(OCC34CCCN3CCC4)nc3c2CCN(c2cccc4cccc(Cl)c24)C3)CCN1C(=O)/C=C/CN1[C@@H]2CC[C@H]1COC2 ; N#CC[C@H]1CN(c2nc(OCC34CCCN3CCC4)nc3c2CCN(c2cccc4cccc(Cl)c24)C3)CCN1C(=O)/C=C/CN1CCS(=O)(=O)CC1 ; N#CC[C@H]1CN(c2nc(OCC34CCCN3CCC4)nc3c2CCN(c2cccc4cccc(Cl)c24)C3)CCN1C(=O)/C=C/CN1CCCCC1] 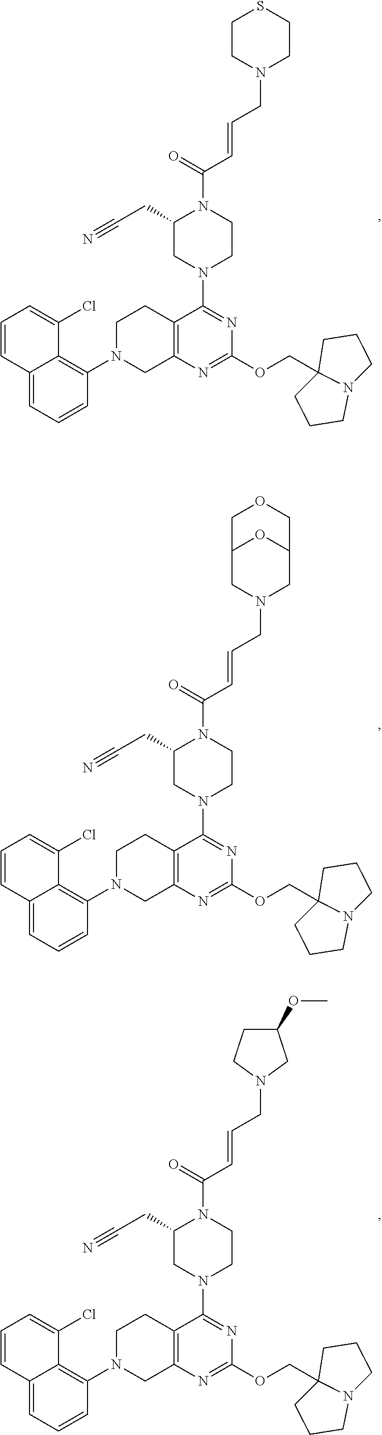 [SMILES: N#CC[C@H]1CN(c2nc(OCC34CCCN3CCC4)nc3c2CCN(c2cccc4cccc(Cl)c24)C3)CCN1C(=O)/C=C/CN1CCSCC1 ; N#CC[C@H]1CN(c2nc(OCC34CCCN3CCC4)nc3c2CCN(c2cccc4cccc(Cl)c24)C3)CCN1C(=O)/C=C/CN1CC2COCC(C1)O2 ; CO[C@@H]1CCN(C/C=C/C(=O)N2CCN(c3nc(OCC45CCCN4CCC5)nc4c3CCN(c3cccc5cccc(Cl)c35)C4)C[C@@H]2CC#N)C1] 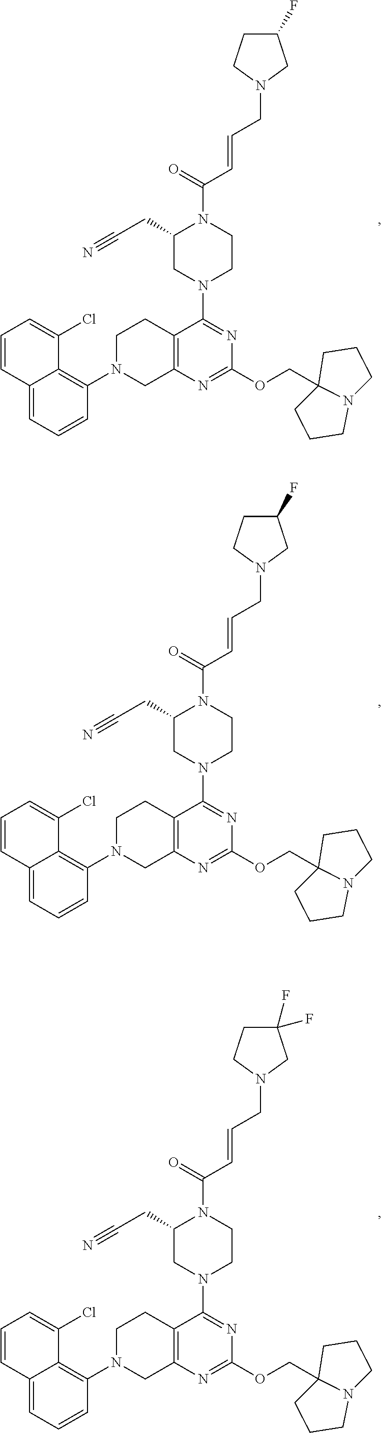 [SMILES: N#CC[C@H]1CN(c2nc(OCC34CCCN3CCC4)nc3c2CCN(c2cccc4cccc(Cl)c24)C3)CCN1C(=O)/C=C/CN1CC[C@H](F)C1 ; N#CC[C@H]1CN(c2nc(OCC34CCCN3CCC4)nc3c2CCN(c2cccc4cccc(Cl)c24)C3)CCN1C(=O)/C=C/CN1CC[C@@H](F)C1 ; N#CC[C@H]1CN(c2nc(OCC34CCCN3CCC4)nc3c2CCN(c2cccc4cccc(Cl)c24)C3)CCN1C(=O)/C=C/CN1CCC(F)(F)C1] 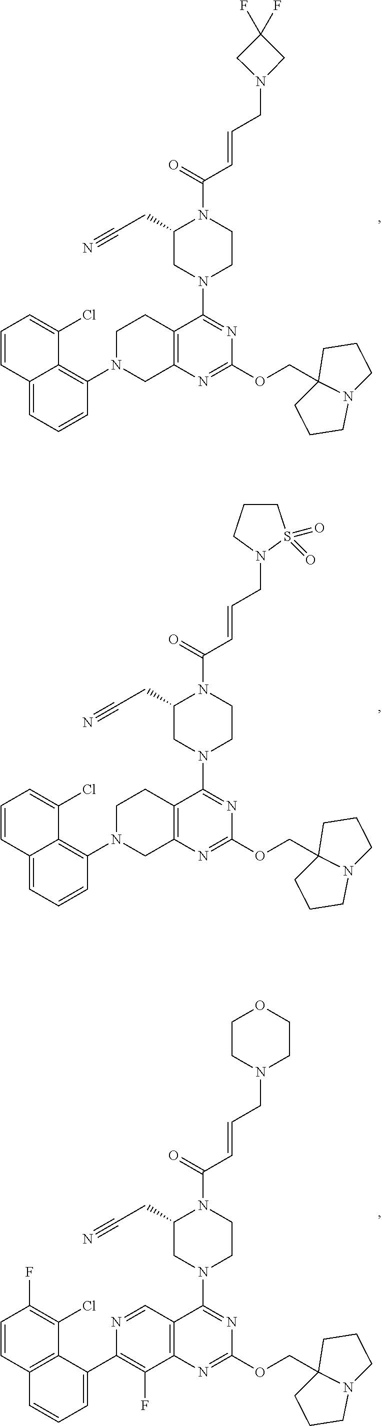 [SMILES: N#CC[C@H]1CN(c2nc(OCC34CCCN3CCC4)nc3c2CCN(c2cccc4cccc(Cl)c24)C3)CCN1C(=O)/C=C/CN1CC(F)(F)C1 ; N#CC[C@H]1CN(c2nc(OCC34CCCN3CCC4)nc3c2CCN(c2cccc4cccc(Cl)c24)C3)CCN1C(=O)/C=C/CN1CCCS1(=O)=O ; N#CC[C@H]1CN(c2nc(OCC34CCCN3CCC4)nc3c(F)c(-c4cccc5ccc(F)c(Cl)c45)ncc23)CCN1C(=O)/C=C/CN1CCOCC1] 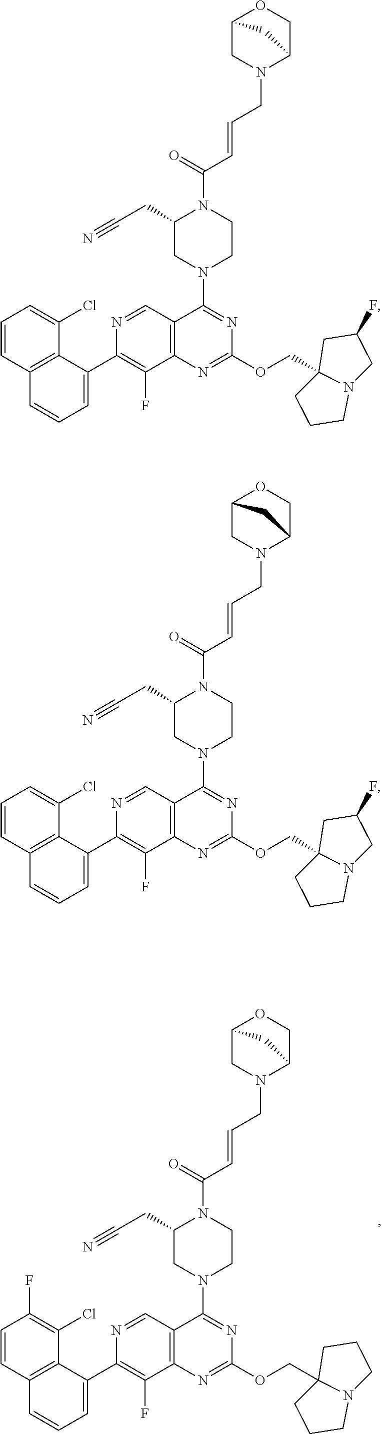 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@@H]2C[C@H]1CO2 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C[C@H]2C[C@@H]1CO2 ; N#CC[C@H]1CN(c2nc(OCC34CCCN3CCC4)nc3c(F)c(-c4cccc5ccc(F)c(Cl)c45)ncc23)CCN1C(=O)/C=C/CN1C[C@@H]2C[C@H]1CO2] 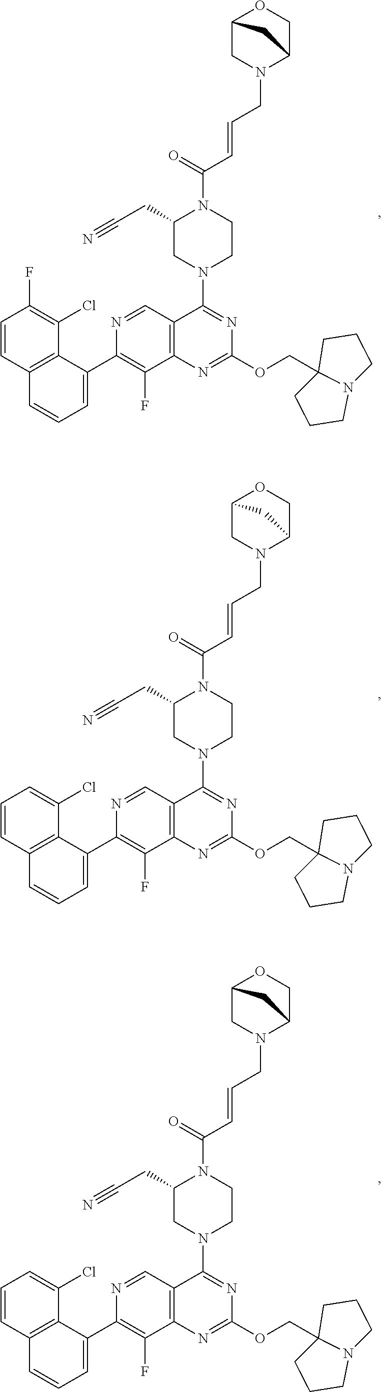 [SMILES: N#CC[C@H]1CN(c2nc(OCC34CCCN3CCC4)nc3c(F)c(-c4cccc5ccc(F)c(Cl)c45)ncc23)CCN1C(=O)/C=C/CN1C[C@H]2C[C@@H]1CO2 ; N#CC[C@H]1CN(c2nc(OCC34CCCN3CCC4)nc3c(F)c(-c4cccc5cccc(Cl)c45)ncc23)CCN1C(=O)/C=C/CN1C[C@@H]2C[C@H]1CO2 ; N#CC[C@H]1CN(c2nc(OCC34CCCN3CCC4)nc3c(F)c(-c4cccc5cccc(Cl)c45)ncc23)CCN1C(=O)/C=C/CN1C[C@H]2C[C@@H]1CO2] 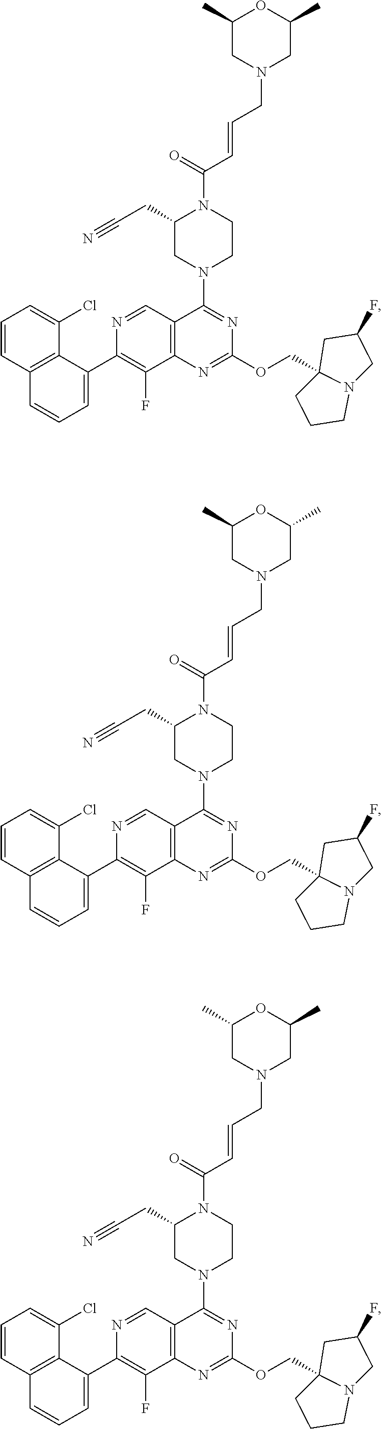 [SMILES: C[C@@H]1CN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C[C@H](C)O1 ; C[C@@H]1CN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C[C@@H](C)O1 ; C[C@H]1CN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C[C@H](C)O1] 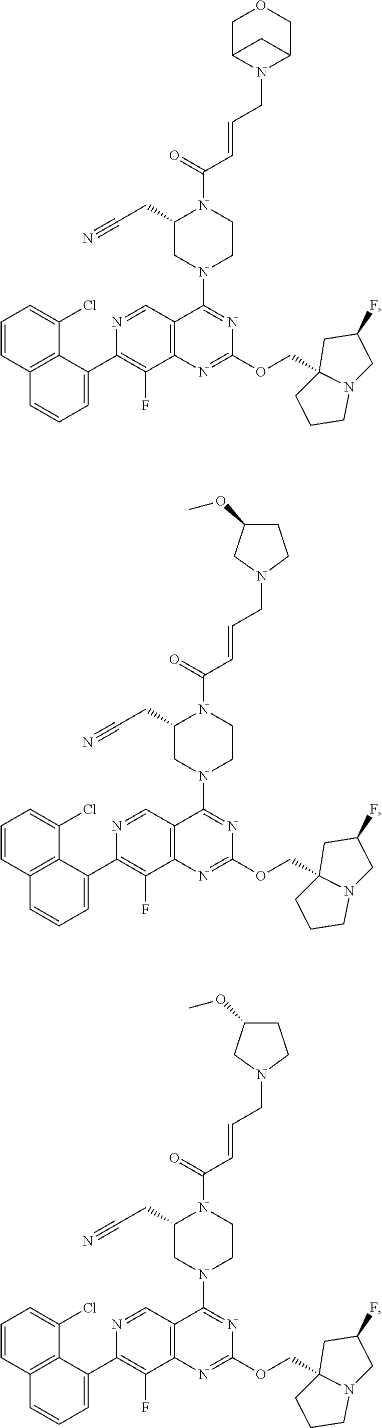 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1C2COCC1C2 ; CO[C@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1 ; CO[C@@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1] 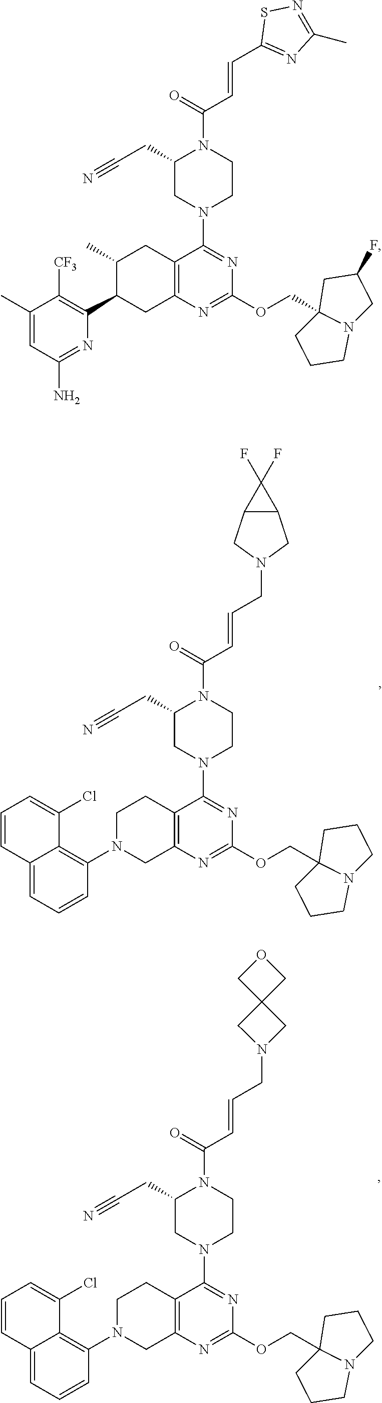 [SMILES: Cc1nsc(/C=C/C(=O)N2CCN(c3nc(OC[C@@]45CCCN4C[C@H](F)C5)nc4c3C[C@@H](C)[C@H](c3nc(N)cc(C)c3C(F)(F)F)C4)C[C@@H]2CC#N)n1 ; N#CC[C@H]1CN(c2nc(OCC34CCCN3CCC4)nc3c2CCN(c2cccc4cccc(Cl)c24)C3)CCN1C(=O)/C=C/CN1CC2C(C1)C2(F)F ; N#CC[C@H]1CN(c2nc(OCC34CCCN3CCC4)nc3c2CCN(c2cccc4cccc(Cl)c24)C3)CCN1C(=O)/C=C/CN1CC2(COC2)C1] 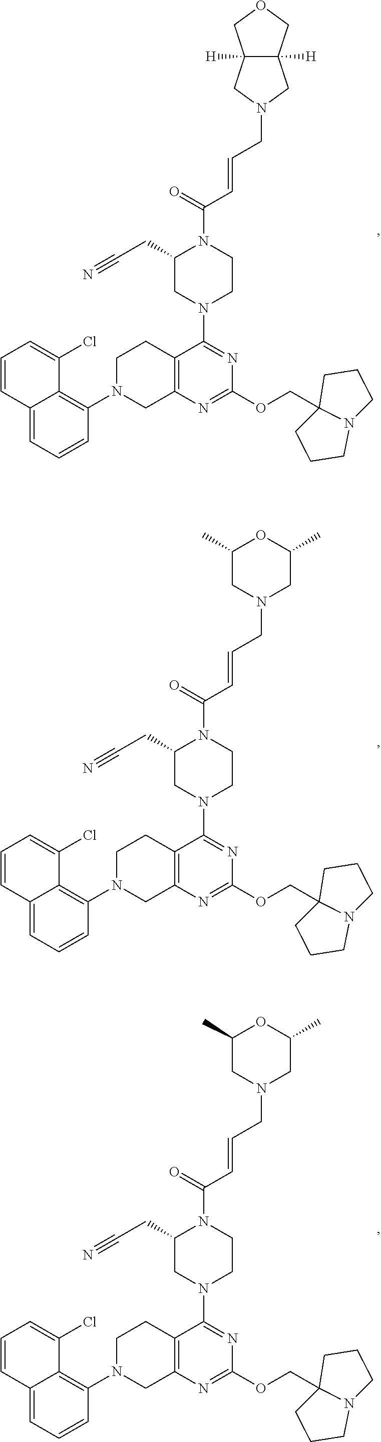 [SMILES: N#CC[C@H]1CN(c2nc(OCC34CCCN3CCC4)nc3c2CCN(c2cccc4cccc(Cl)c24)C3)CCN1C(=O)/C=C/CN1C[C@H]2COC[C@H]2C1 ; C[C@@H]1CN(C/C=C/C(=O)N2CCN(c3nc(OCC45CCCN4CCC5)nc4c3CCN(c3cccc5cccc(Cl)c35)C4)C[C@@H]2CC#N)C[C@H](C)O1 ; C[C@@H]1CN(C/C=C/C(=O)N2CCN(c3nc(OCC45CCCN4CCC5)nc4c3CCN(c3cccc5cccc(Cl)c35)C4)C[C@@H]2CC#N)C[C@@H](C)O1] 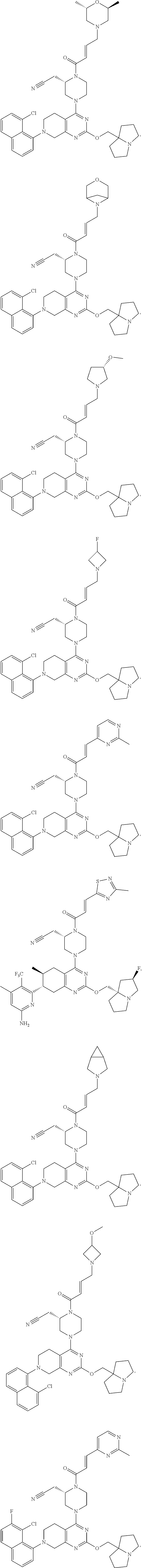 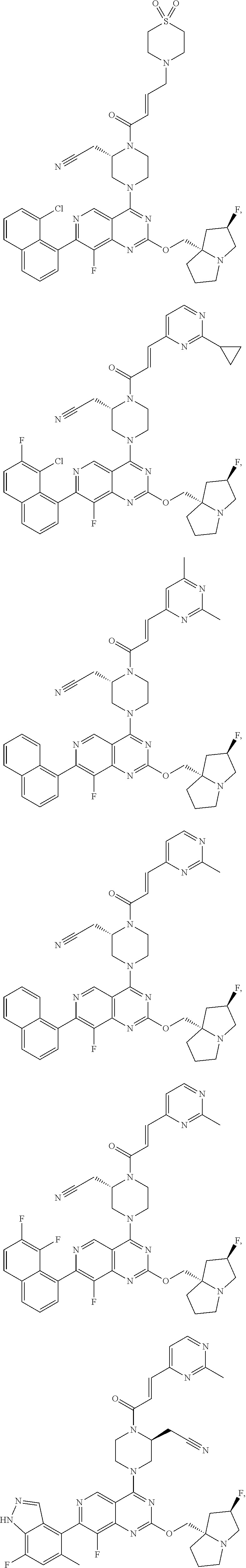 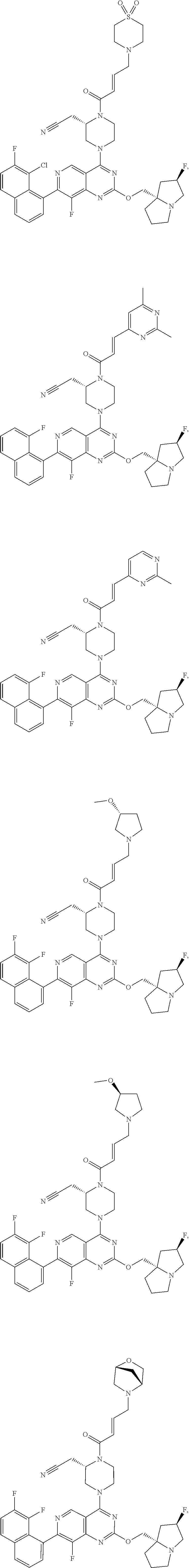 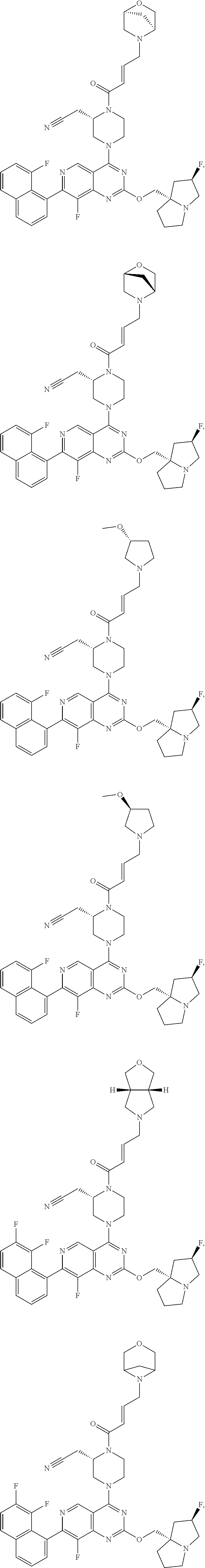 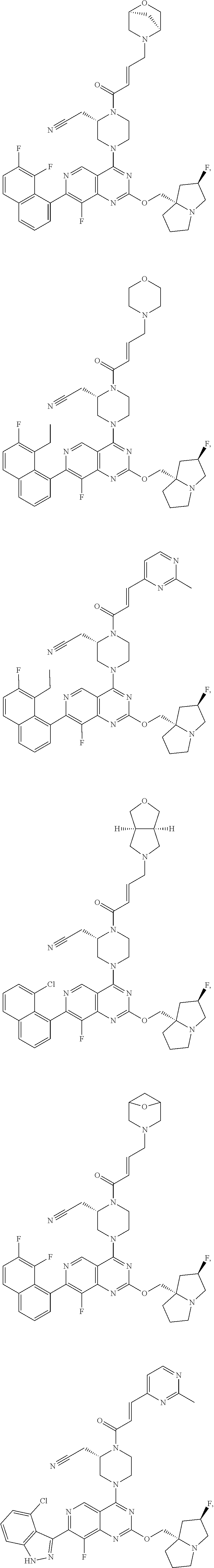 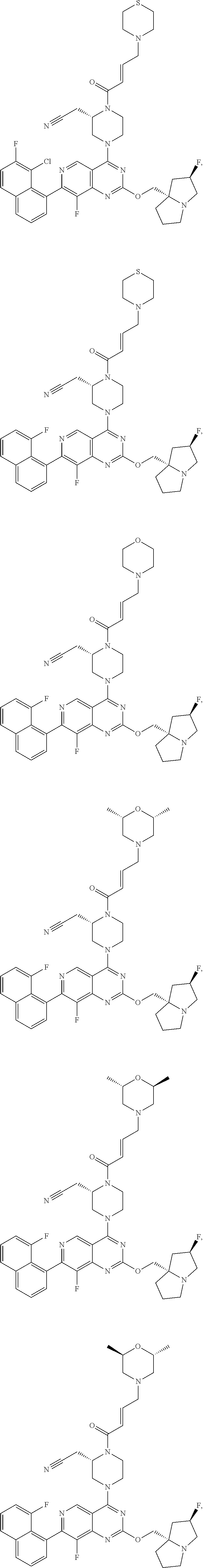 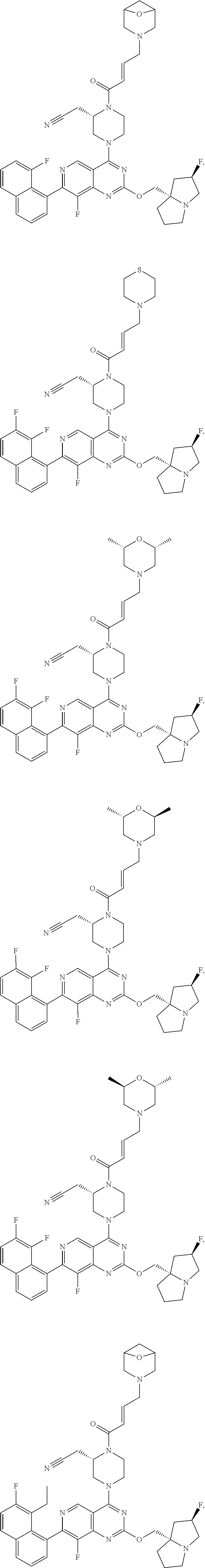 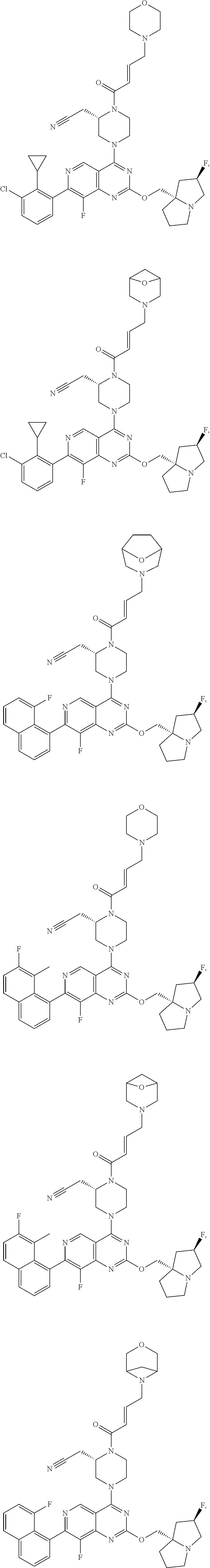 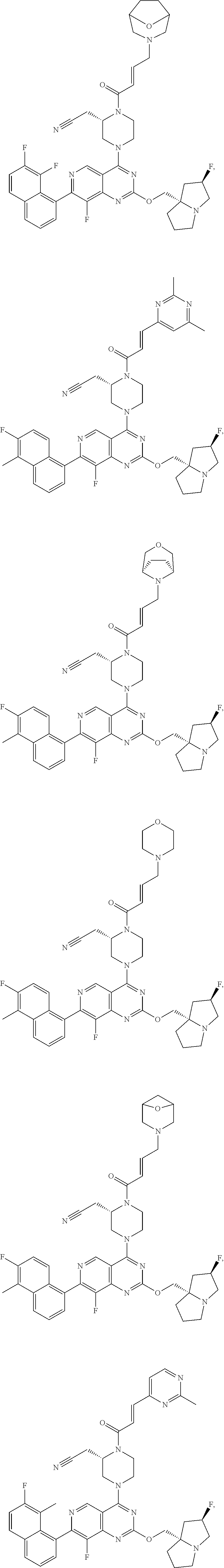 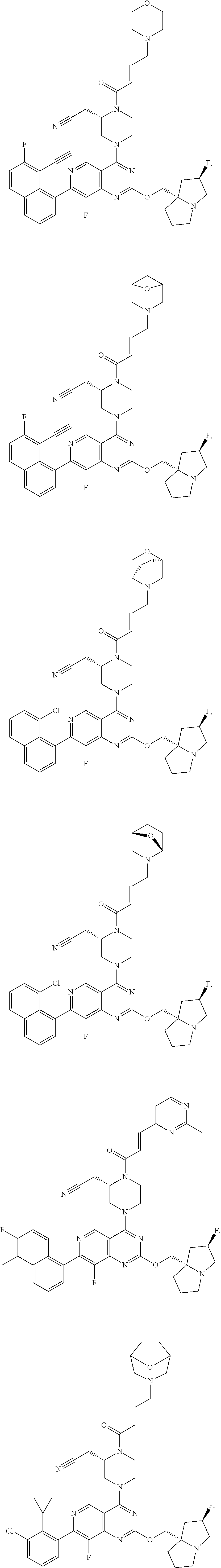 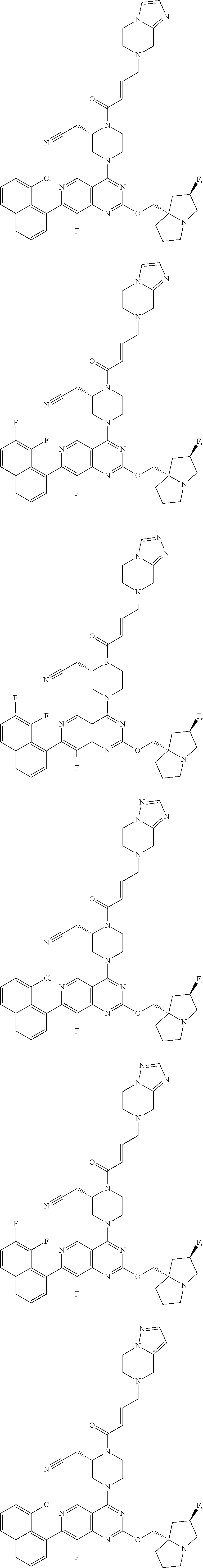 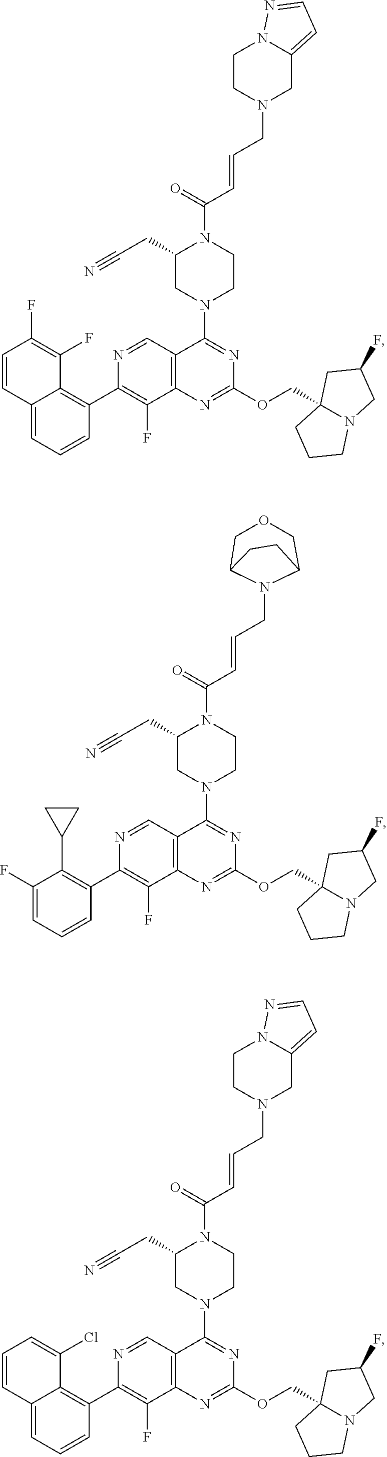 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCn2nccc2C1 ; N#CC[C@H]1CN(c2nc(OC[C@@]34CCCN3C[C@H](F)C4)nc3c(F)c(-c4cccc(F)c4C4CC4)ncc23)CCN1C(=O)/C=C/CN1C2CCC1COC2 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCn2nccc2C1] 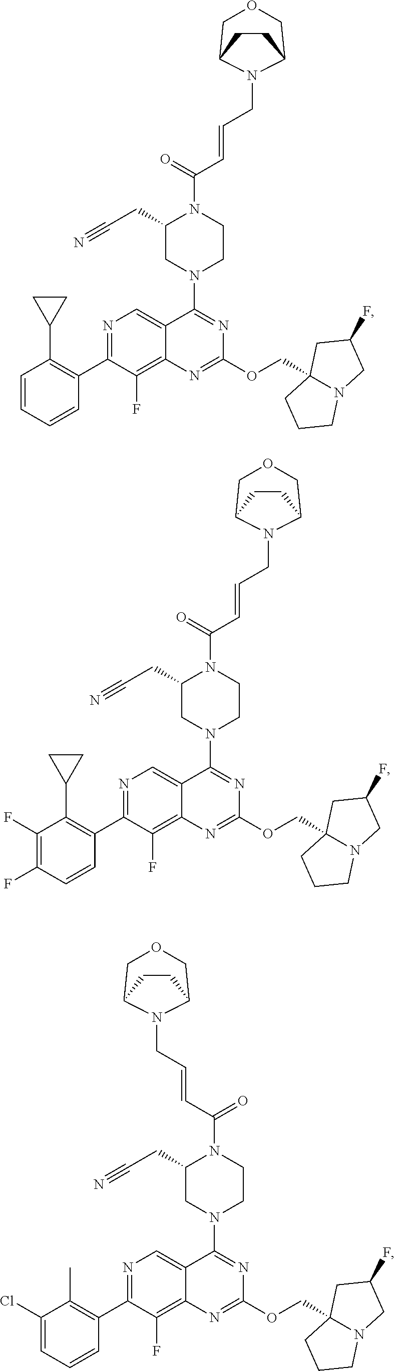 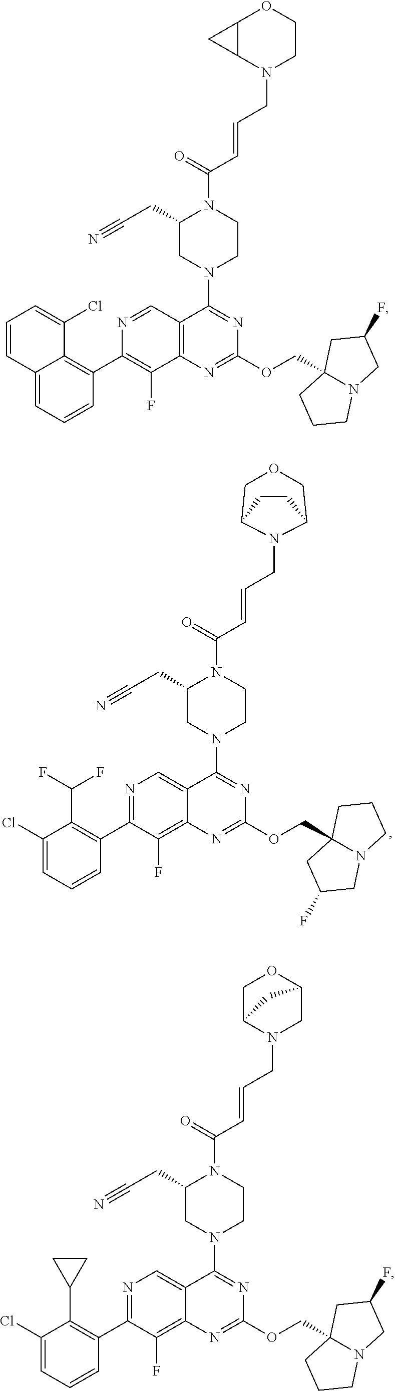 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCOC2CC21 ; N#CC[C@H]1CN(c2nc(OC[C@@]34CCCN3C[C@H](F)C4)nc3c(F)c(-c4cccc(Cl)c4C(F)F)ncc23)CCN1C(=O)/C=C/CN1[C@@H]2CC[C@H]1COC2 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1C[C@H]2C[C@@H]1CO2] 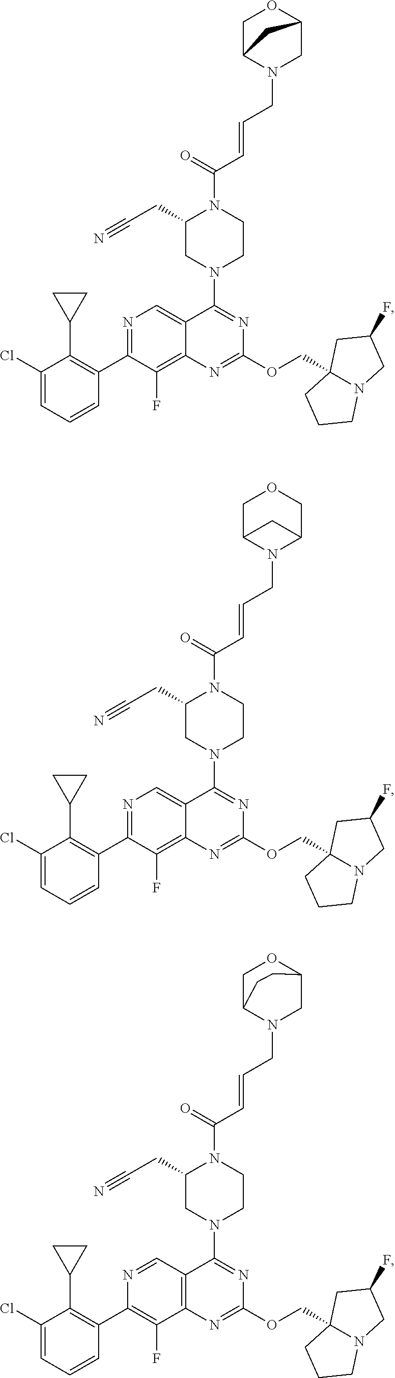 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1C[C@@H]2C[C@H]1CO2 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1C2COCC1C2 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CC2CCC1CO2] 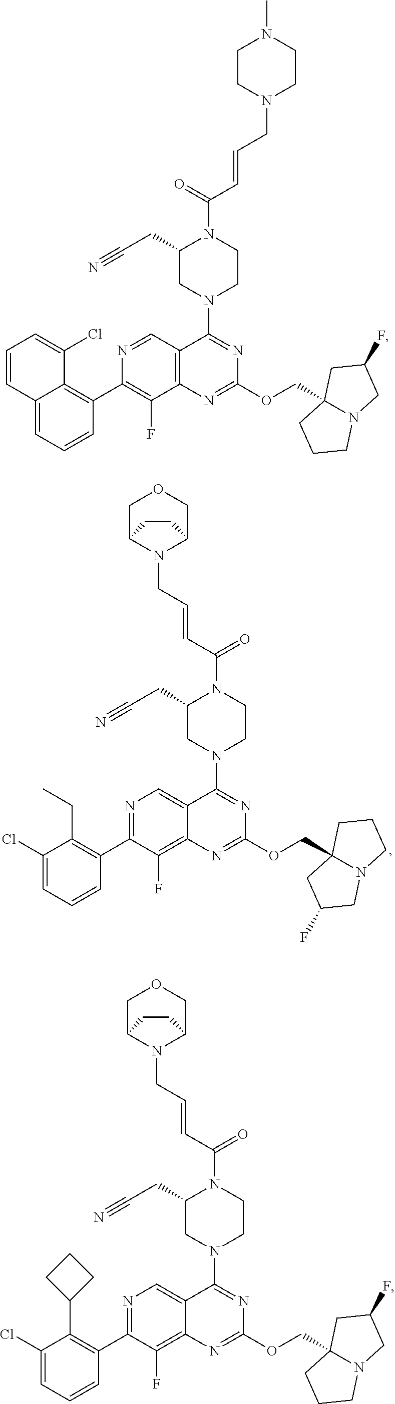 [SMILES: CN1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)CC1 ; CCc1c(Cl)cccc1-c1ncc2c(N3CCN(C(=O)/C=C/CN4[C@@H]5CC[C@H]4COC5)[C@@H](CC#N)C3)nc(OC[C@@]34CCCN3C[C@H](F)C4)nc2c1F ; N#CC[C@H]1CN(c2nc(OC[C@@]34CCCN3C[C@H](F)C4)nc3c(F)c(-c4cccc(Cl)c4C4CCC4)ncc23)CCN1C(=O)/C=C/CN1[C@@H]2CC[C@H]1COC2] 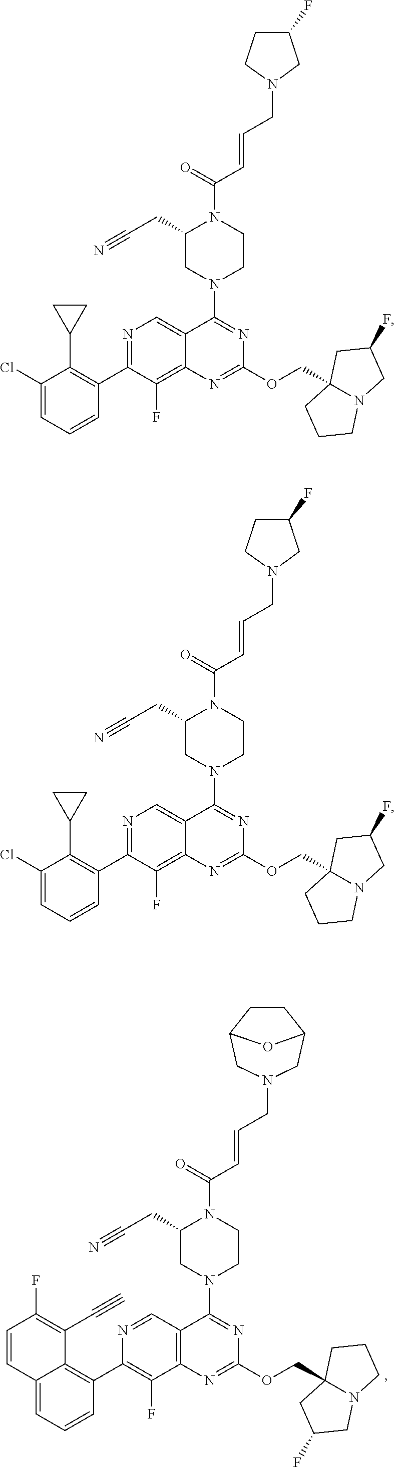 [SMILES: N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CC[C@H](F)C1 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CC[C@@H](F)C1 ; C#Cc1c(F)ccc2cccc(-c3ncc4c(N5CCN(C(=O)/C=C/CN6CC7CCC(C6)O7)[C@@H](CC#N)C5)nc(OC[C@@]56CCCN5C[C@H](F)C6)nc4c3F)c12] 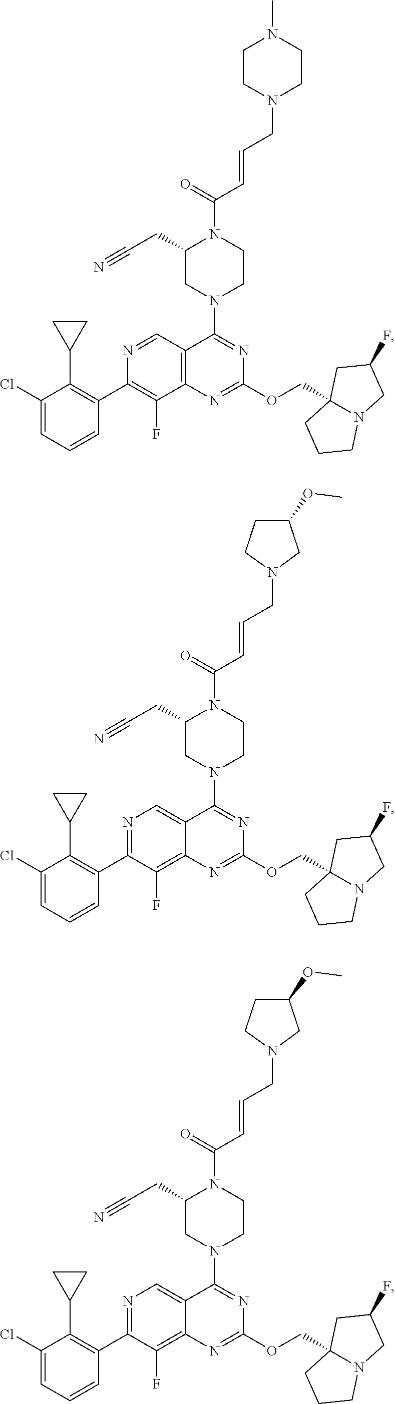 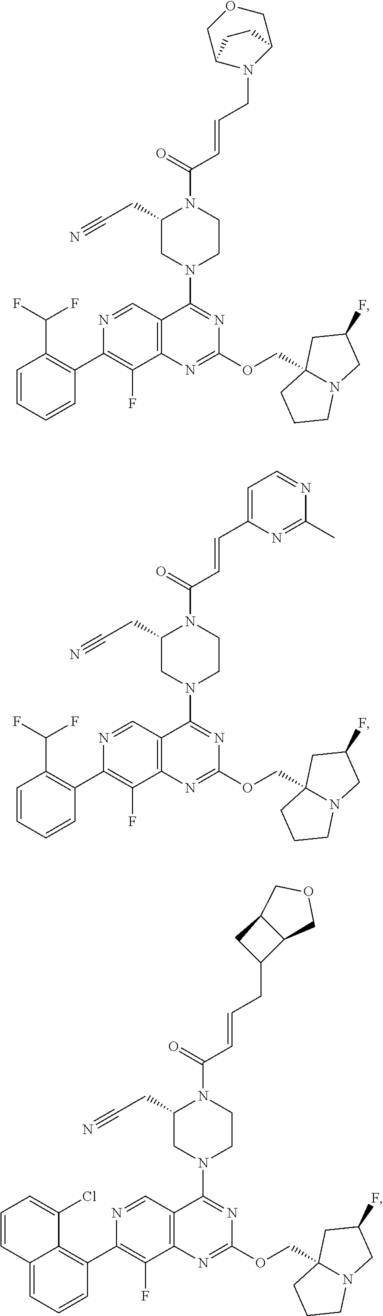 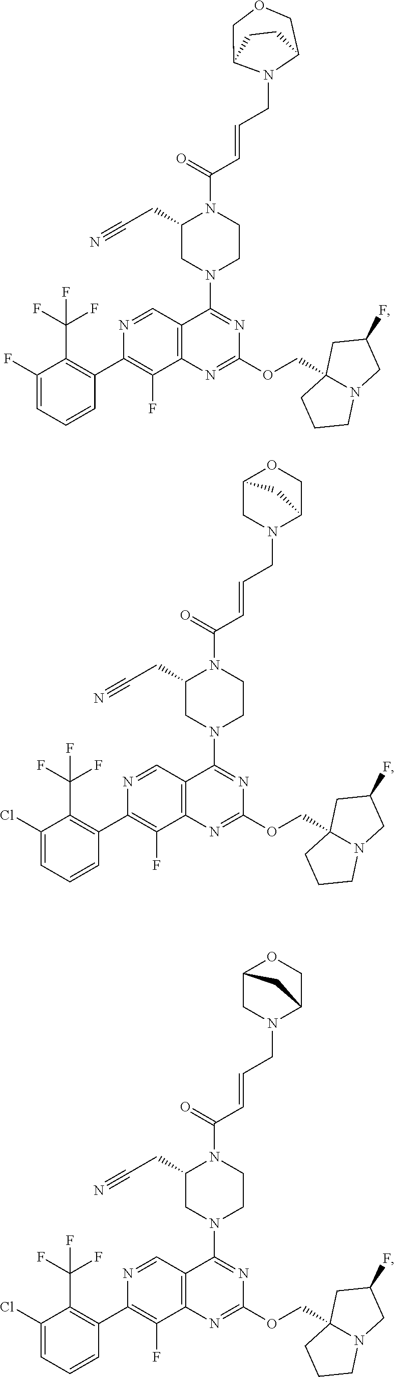 [SMILES: N#CC[C@H]1CN(c2nc(OC[C@@]34CCCN3C[C@H](F)C4)nc3c(F)c(-c4cccc(F)c4C(F)(F)F)ncc23)CCN1C(=O)/C=C/CN1[C@@H]2CC[C@H]1COC2 ; N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1C[C@@H]2C[C@H]1CO2 ; N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1C[C@H]2C[C@@H]1CO2] 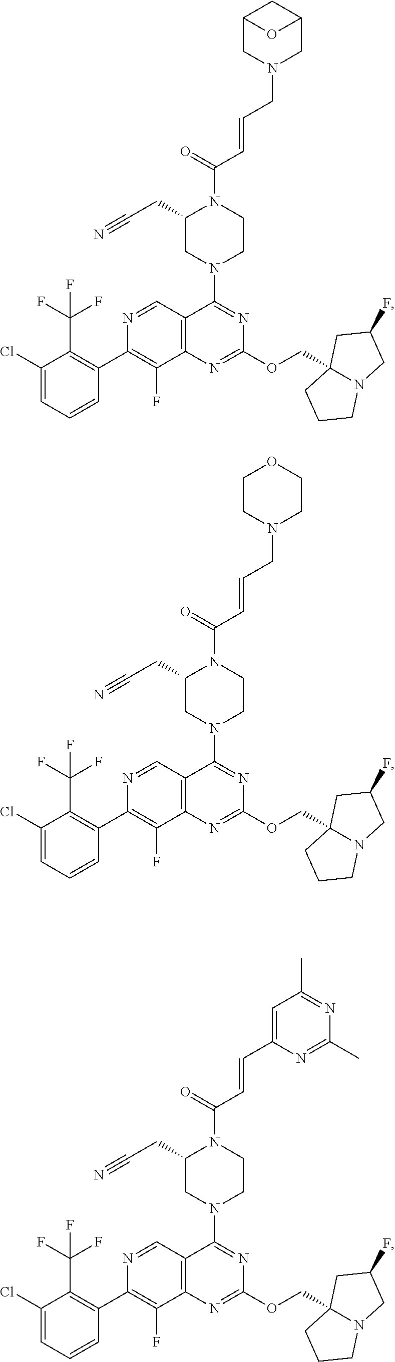 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CC2CC(C1)O2 ; N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CCOCC1 ; Cc1cc(/C=C/C(=O)N2CCN(c3nc(OC[C@@]45CCCN4C[C@H](F)C5)nc4c(F)c(-c5cccc(Cl)c5C(F)(F)F)ncc34)C[C@@H]2CC#N)nc(C)n1] 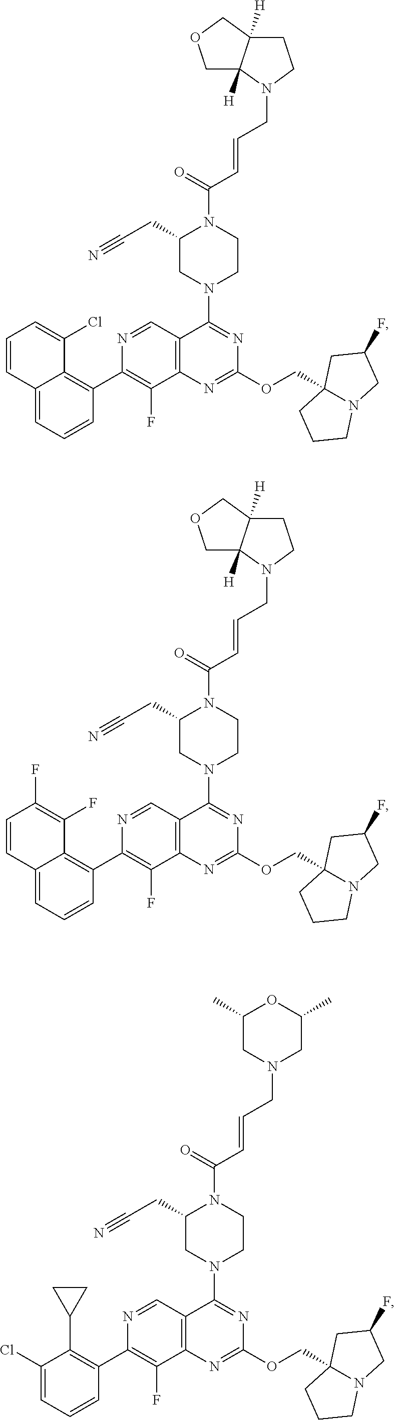 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC[C@@H]2COC[C@H]21 ; N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CC[C@@H]2COC[C@H]21 ; C[C@@H]1CN(C/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)C[C@H](C)O1] 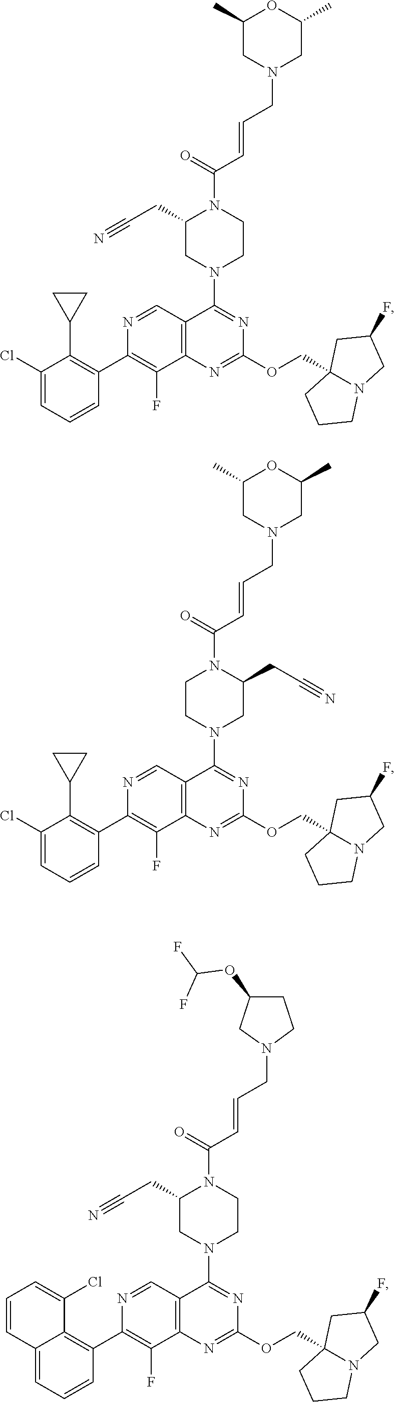 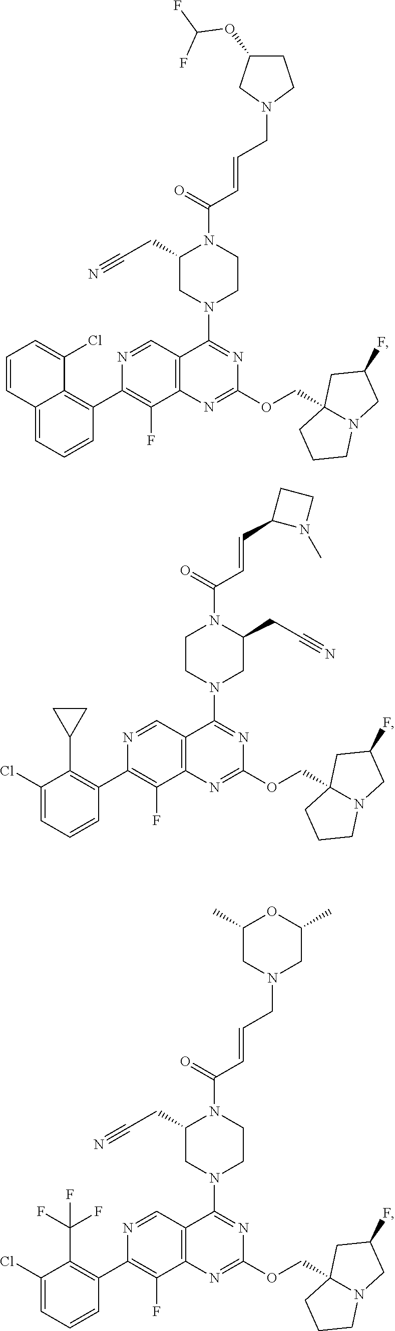 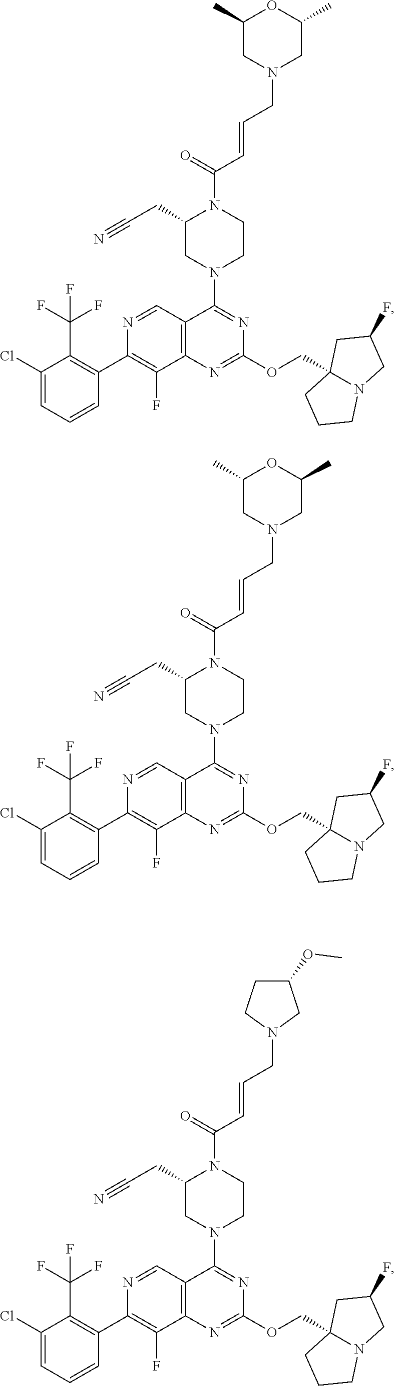 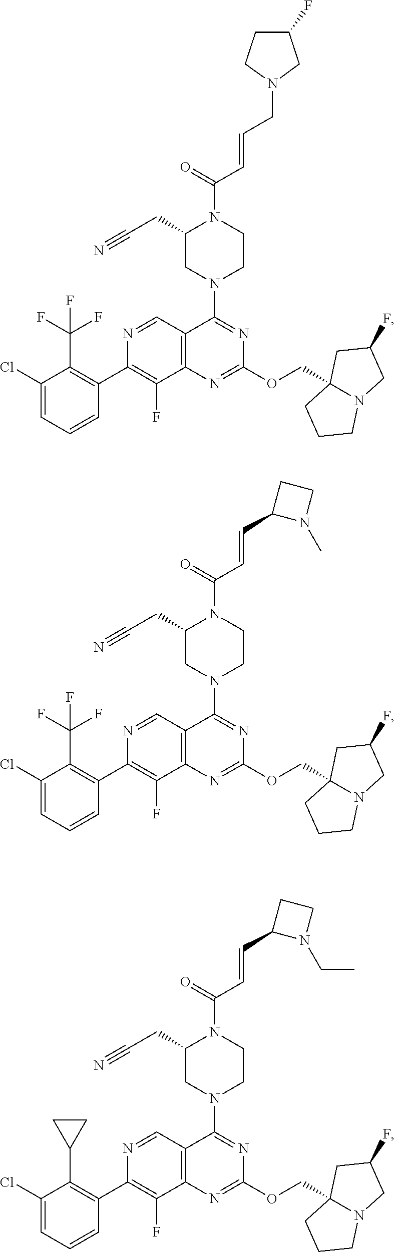 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CC[C@H](F)C1 ; CN1CC[C@@H]1/C=C/C(=O)N1CCN(c2nc(OC[C@@]34CCCN3C[C@H](F)C4)nc3c(F)c(-c4cccc(Cl)c4C(F)(F)F)ncc23)C[C@@H]1CC#N ; CCN1CC[C@@H]1/C=C/C(=O)N1CCN(c2nc(OC[C@@]34CCCN3C[C@H](F)C4)nc3c(F)c(-c4cccc(Cl)c4C4CC4)ncc23)C[C@@H]1CC#N] 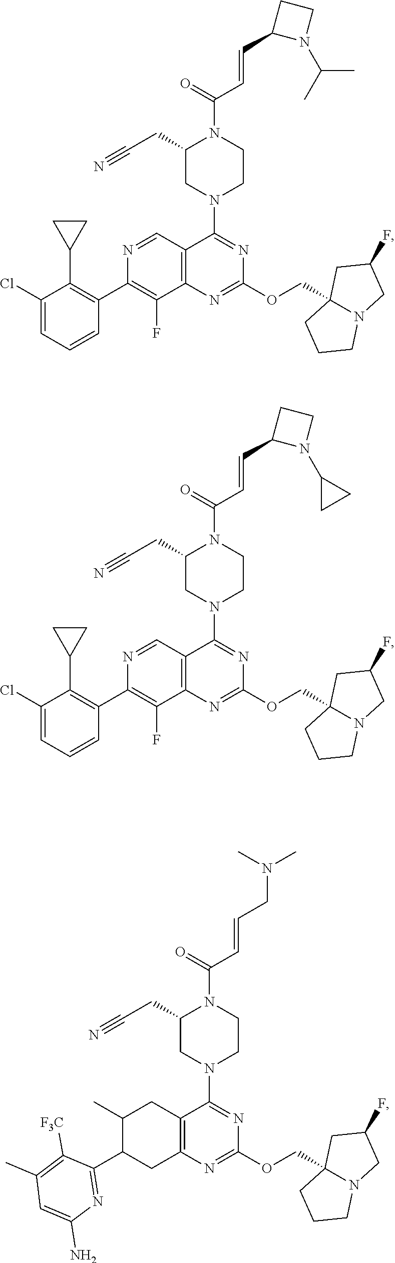 [SMILES: CC(C)N1CC[C@@H]1/C=C/C(=O)N1CCN(c2nc(OC[C@@]34CCCN3C[C@H](F)C4)nc3c(F)c(-c4cccc(Cl)c4C4CC4)ncc23)C[C@@H]1CC#N ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/[C@H]1CCN1C1CC1 ; Cc1cc(N)nc(C2Cc3nc(OC[C@@]45CCCN4C[C@H](F)C5)nc(N4CCN(C(=O)/C=C/CN(C)C)[C@@H](CC#N)C4)c3CC2C)c1C(F)(F)F] 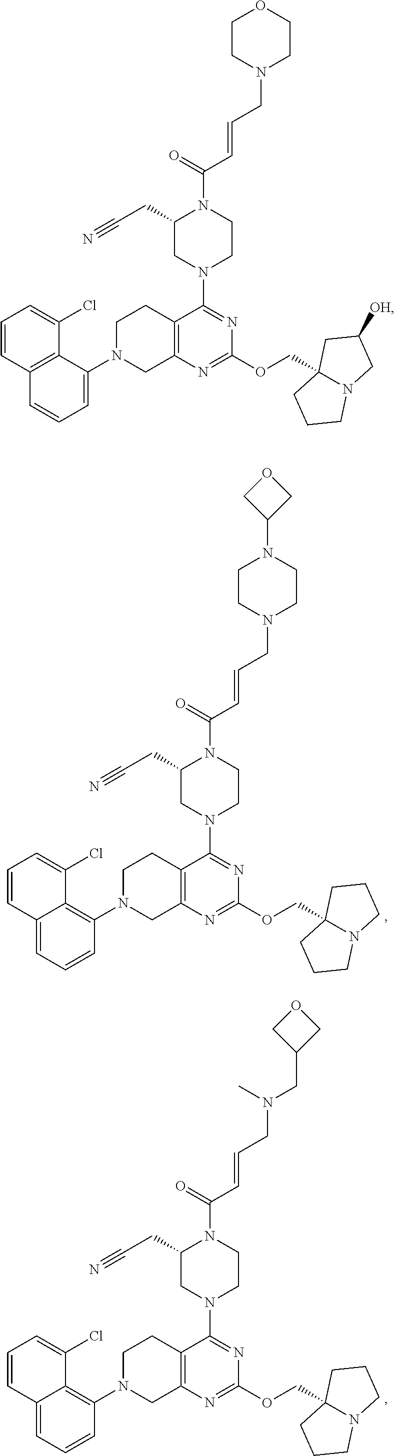 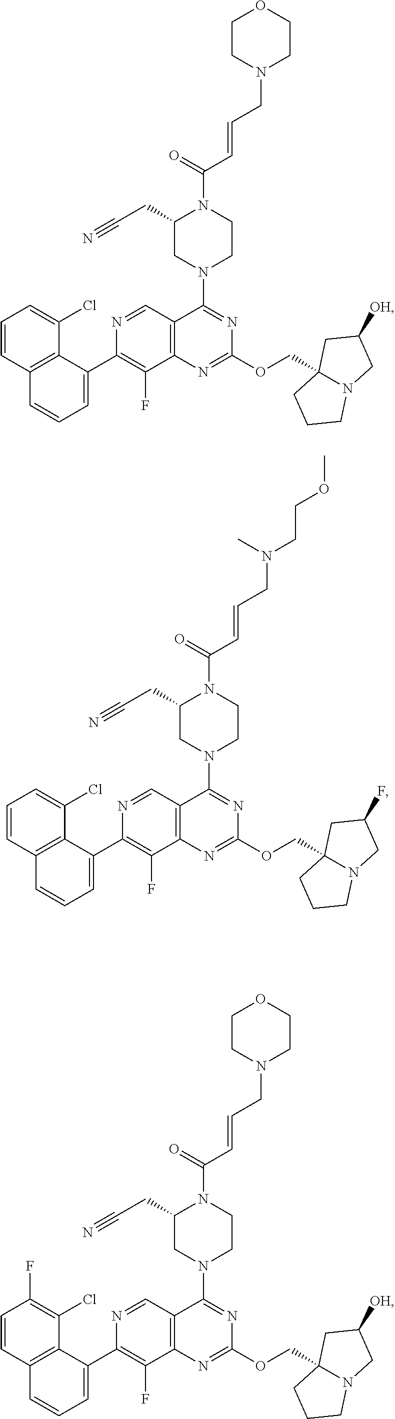 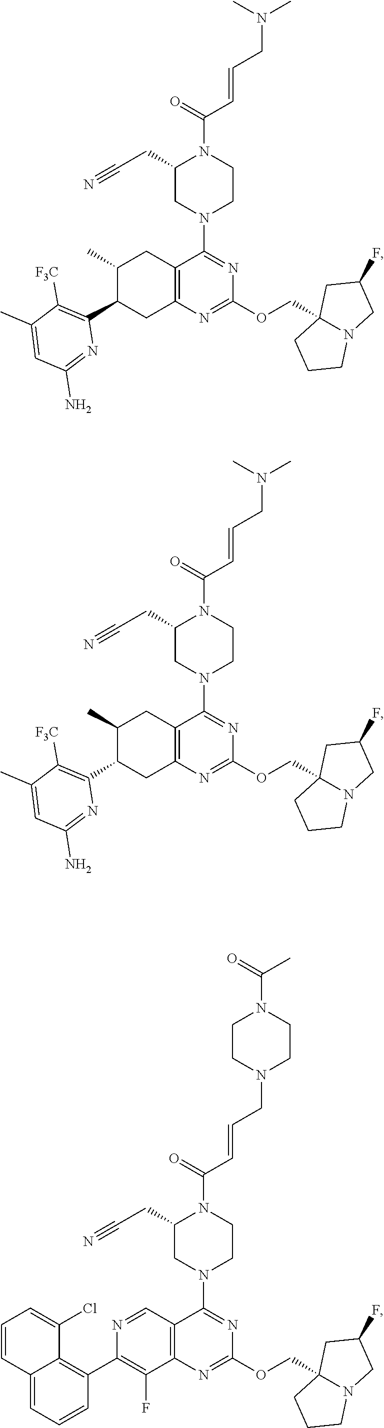 [SMILES: Cc1cc(N)nc([C@@H]2Cc3nc(OC[C@@]45CCCN4C[C@H](F)C5)nc(N4CCN(C(=O)/C=C/CN(C)C)[C@@H](CC#N)C4)c3C[C@H]2C)c1C(F)(F)F ; Cc1cc(N)nc([C@H]2Cc3nc(OC[C@@]45CCCN4C[C@H](F)C5)nc(N4CCN(C(=O)/C=C/CN(C)C)[C@@H](CC#N)C4)c3C[C@@H]2C)c1C(F)(F)F ; CC(=O)N1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)CC1] 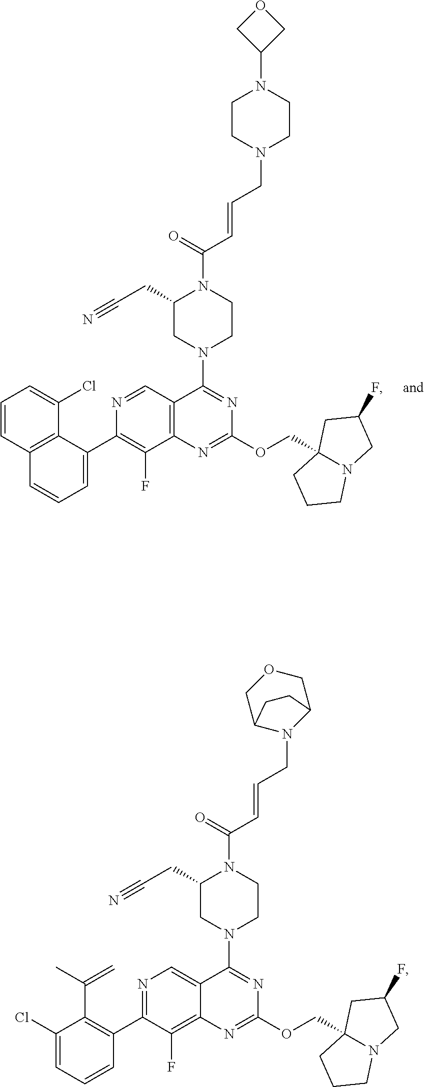 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCN(C2COC2)CC1 ; C=C(C)c1c(Cl)cccc1-c1ncc2c(N3CCN(C(=O)/C=C/CN4C5CCC4COC5)[C@@H](CC#N)C3)nc(OC[C@@]34CCCN3C[C@H](F)C4)nc2c1F]
or a salt thereof, and/or an isotopologue thereof.

115. The compound of any one of claims 1 to 113, wherein the compound is selected from the group consisting of: 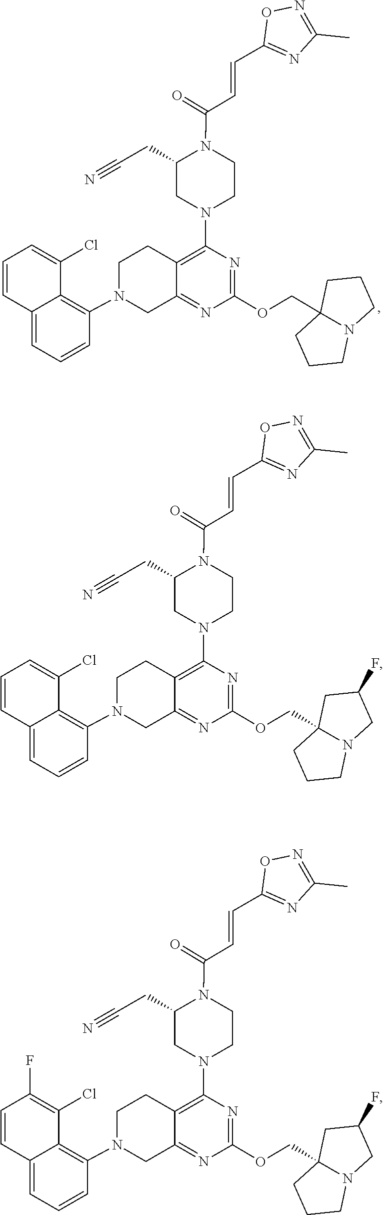 [SMILES: Cc1noc(/C=C/C(=O)N2CCN(c3nc(OCC45CCCN4CCC5)nc4c3CCN(c3cccc5cccc(Cl)c35)C4)C[C@@H]2CC#N)n1 ; Cc1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1 ; Cc1noc(/C=C/C(=O)N2CCN(c3nc(OC[C@@]45CCCN4C[C@H](F)C5)nc4c3CCN(c3cccc5ccc(F)c(Cl)c35)C4)C[C@@H]2CC#N)n1] 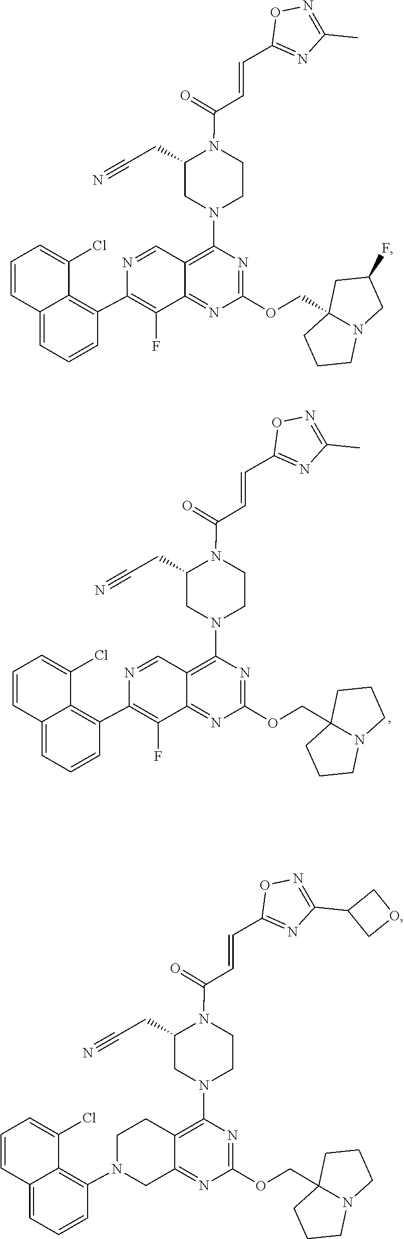 [SMILES: Cc1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; Cc1noc(/C=C/C(=O)N2CCN(c3nc(OCC45CCCN4CCC5)nc4c(F)c(-c5cccc6cccc(Cl)c56)ncc34)C[C@@H]2CC#N)n1 ; N#CC[C@H]1CN(c2nc(OCC34CCCN3CCC4)nc3c2CCN(c2cccc4cccc(Cl)c24)C3)CCN1C(=O)/C=C/c1nc(C2COC2)no1] 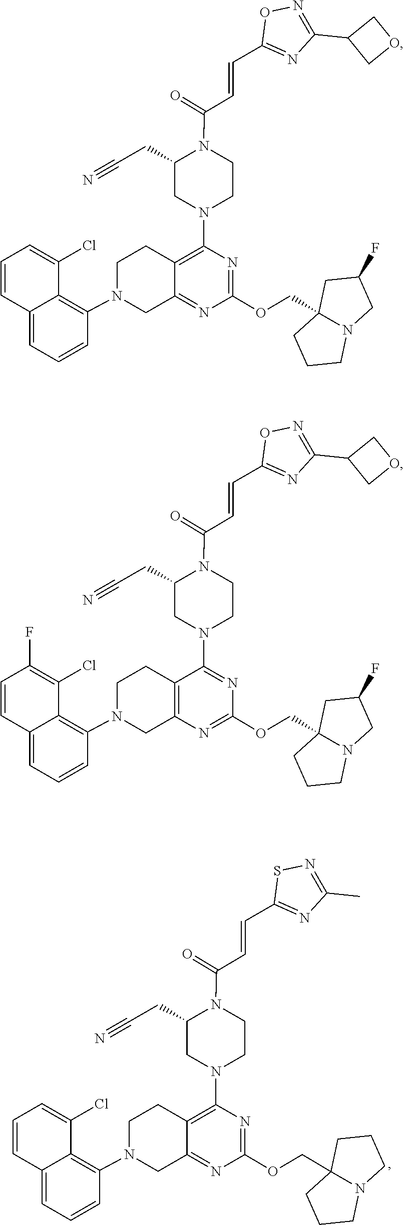 [SMILES: N#CC[C@H]1CN(c2nc(OC[C@@]34CCCN3C[C@H](F)C4)nc3c2CCN(c2cccc4cccc(Cl)c24)C3)CCN1C(=O)/C=C/c1nc(C2COC2)no1 ; N#CC[C@H]1CN(c2nc(OC[C@@]34CCCN3C[C@H](F)C4)nc3c2CCN(c2cccc4ccc(F)c(Cl)c24)C3)CCN1C(=O)/C=C/c1nc(C2COC2)no1 ; Cc1nsc(/C=C/C(=O)N2CCN(c3nc(OCC45CCCN4CCC5)nc4c3CCN(c3cccc5cccc(Cl)c35)C4)C[C@@H]2CC#N)n1] 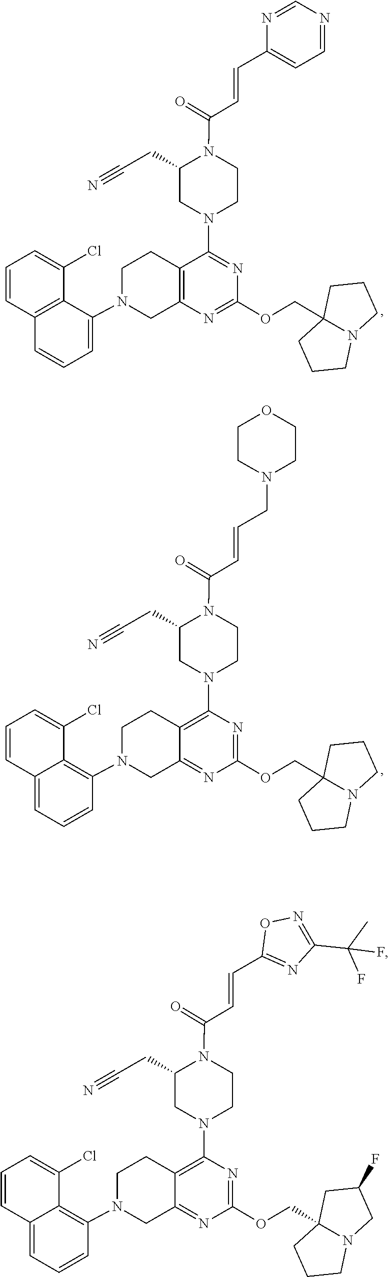 [SMILES: N#CC[C@H]1CN(c2nc(OCC34CCCN3CCC4)nc3c2CCN(c2cccc4cccc(Cl)c24)C3)CCN1C(=O)/C=C/c1ccncn1 ; N#CC[C@H]1CN(c2nc(OCC34CCCN3CCC4)nc3c2CCN(c2cccc4cccc(Cl)c24)C3)CCN1C(=O)/C=C/CN1CCOCC1 ; CC(F)(F)c1noc(/C=C/C(=O)N2CCN(c3nc(OC[C@@]45CCCN4C[C@H](F)C5)nc4c3CCN(c3cccc5cccc(Cl)c35)C4)C[C@@H]2CC#N)n1] 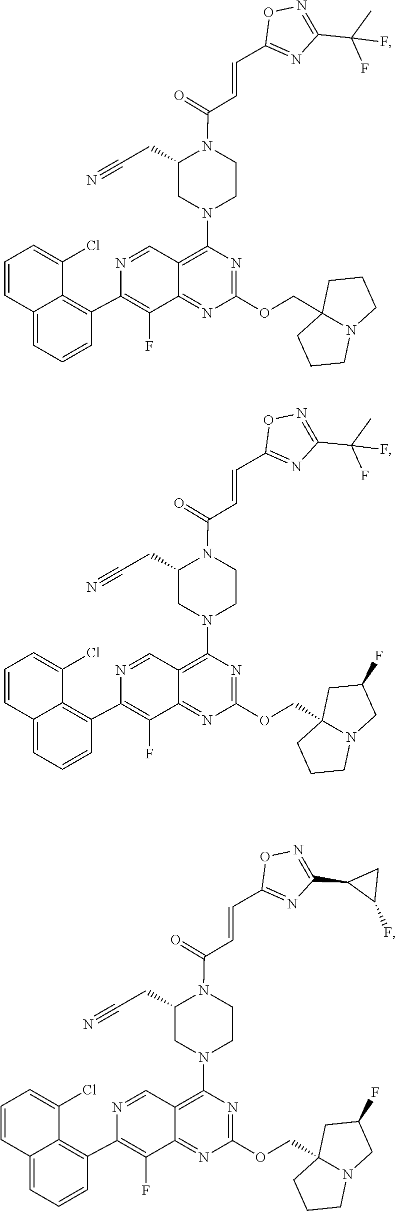 [SMILES: CC(F)(F)c1noc(/C=C/C(=O)N2CCN(c3nc(OCC45CCCN4CCC5)nc4c(F)c(-c5cccc6cccc(Cl)c56)ncc34)C[C@@H]2CC#N)n1 ; CC(F)(F)c1noc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)n1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/c1nc([C@H]2C[C@@H]2F)no1] 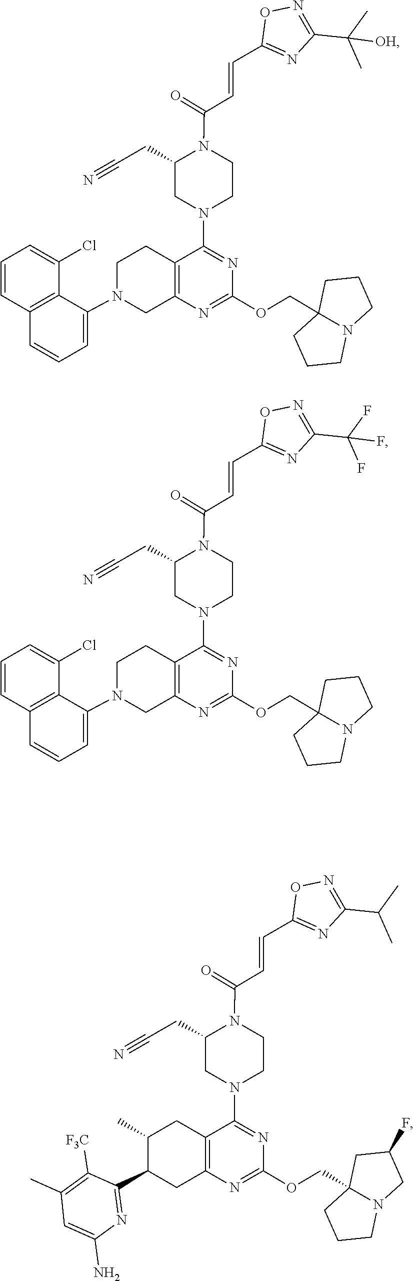 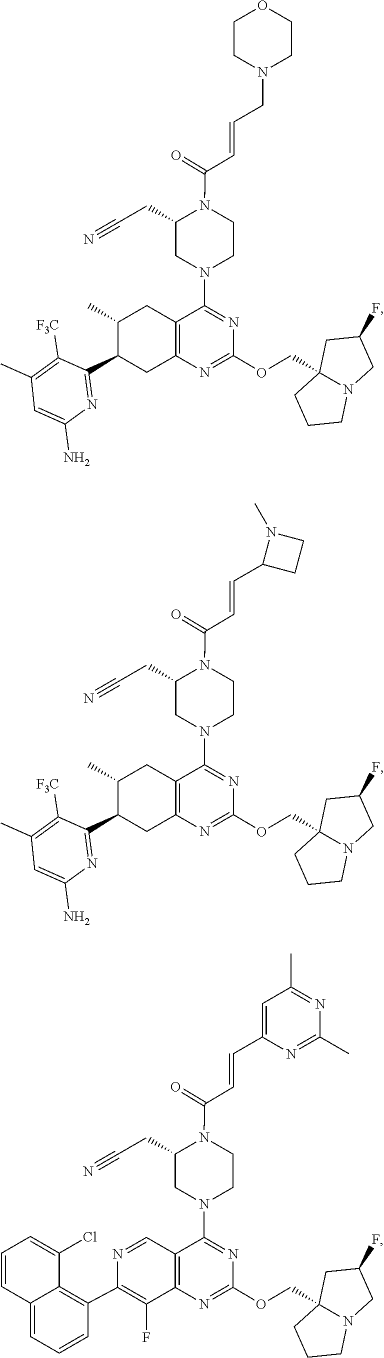 [SMILES: Cc1cc(N)nc([C@@H]2Cc3nc(OC[C@@]45CCCN4C[C@H](F)C5)nc(N4CCN(C(=O)/C=C/CN5CCOCC5)[C@@H](CC#N)C4)c3C[C@H]2C)c1C(F)(F)F ; Cc1cc(N)nc([C@@H]2Cc3nc(OC[C@@]45CCCN4C[C@H](F)C5)nc(N4CCN(C(=O)/C=C/C5CCN5C)[C@@H](CC#N)C4)c3C[C@H]2C)c1C(F)(F)F ; Cc1cc(/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)nc(C)n1] 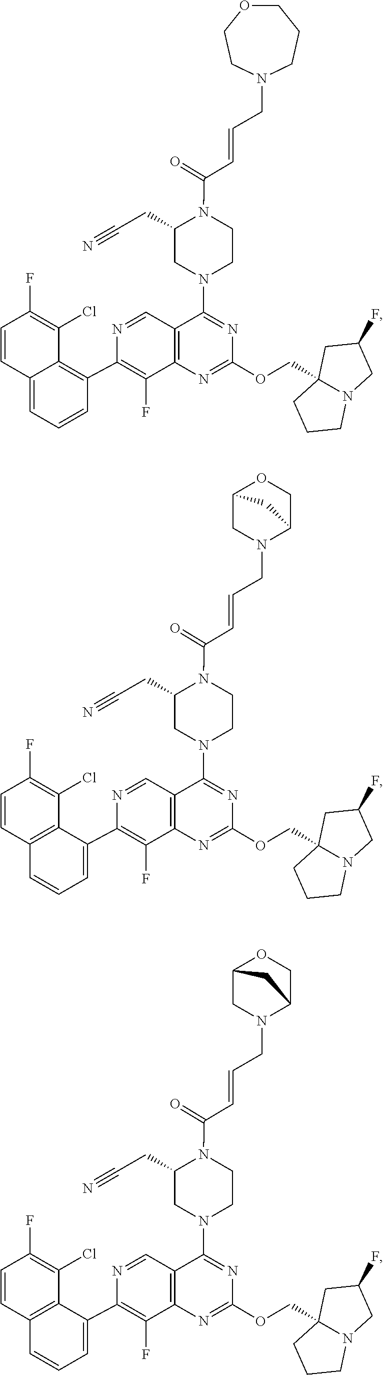 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCCOCC1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@@H]2C[C@H]1CO2 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@H]2C[C@@H]1CO2] 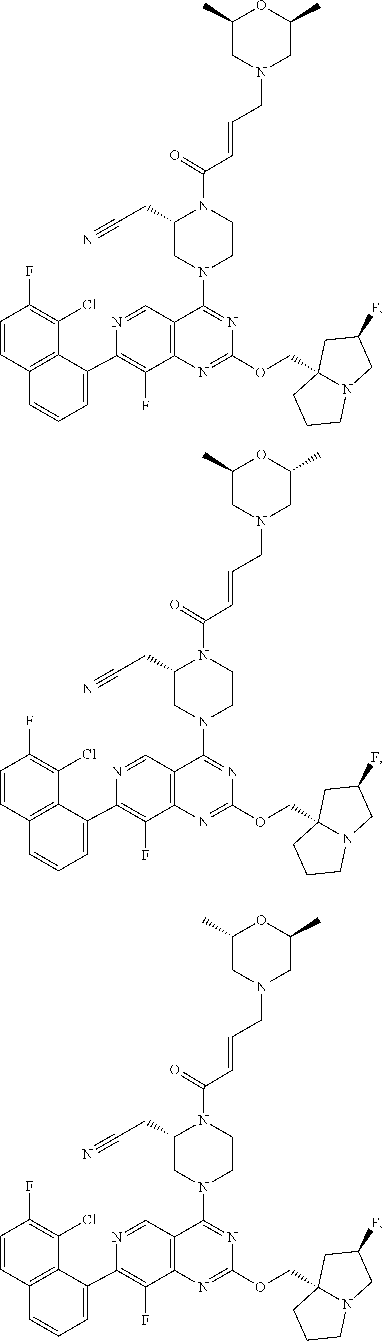 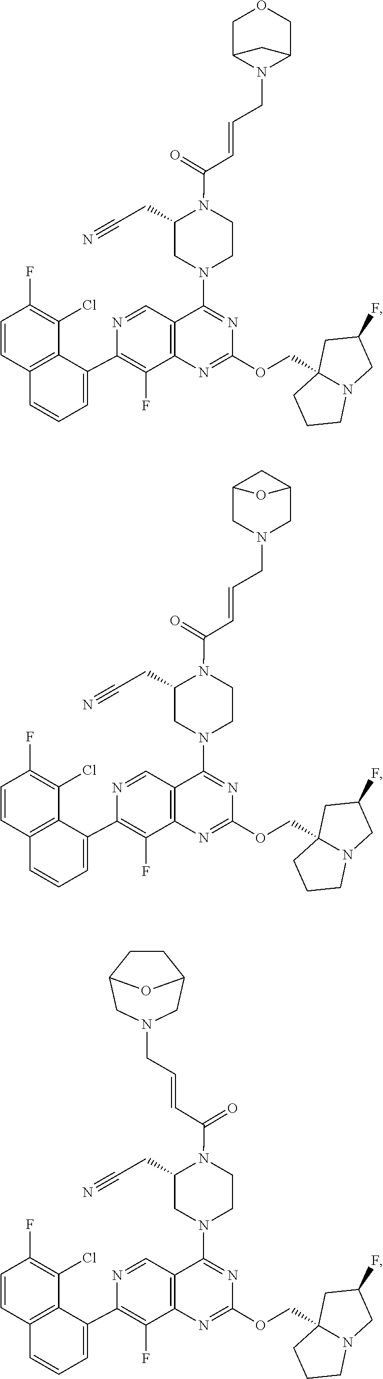 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C2COCC1C2 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2CC(C1)O2 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CC2CCC(C1)O2] 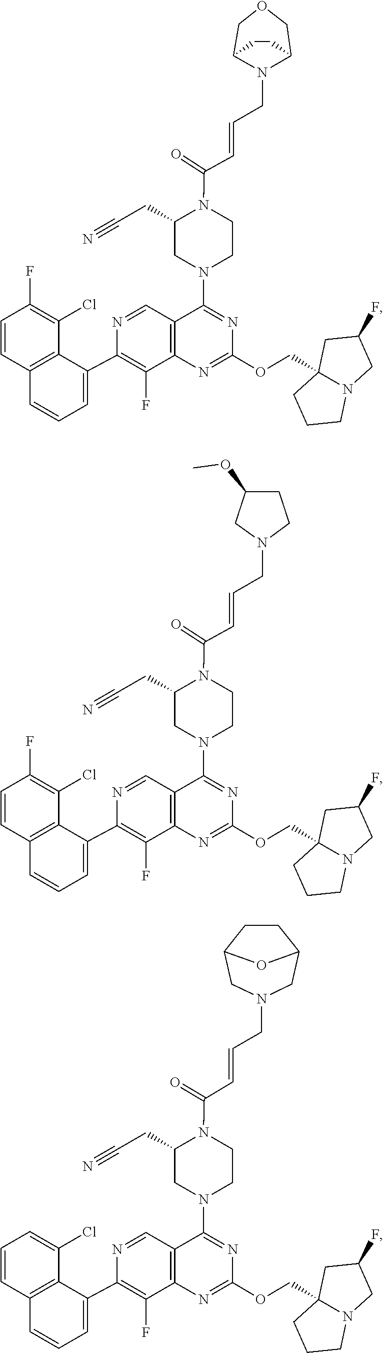 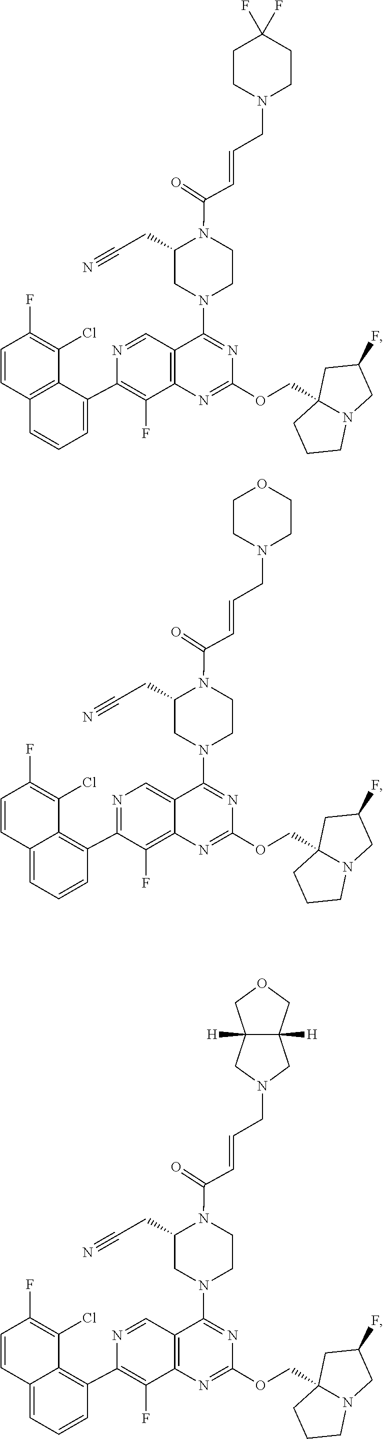 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCC(F)(F)CC1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1CCOCC1 ; N#CC[C@H]1CN(c2nc(OC[C@@]34CCCN3C[C@H](F)C4)nc3c(F)c(-c4cccc5ccc(F)c(Cl)c45)ncc23)CCN1C(=O)/C=C/CN1C[C@H]2COC[C@H]2C1] 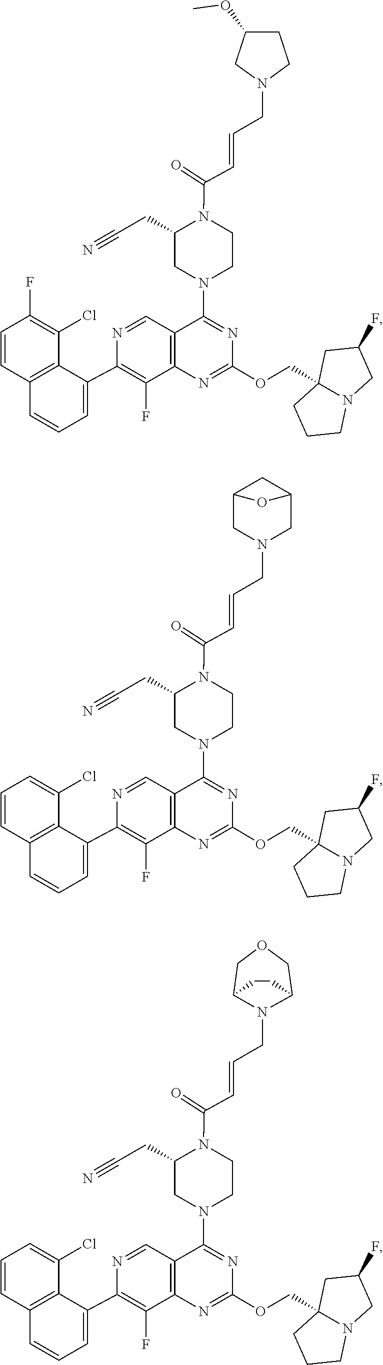 [SMILES: CO[C@@H]1CCN(C/C=C/C(=O)N2CCN(c3nc(OC[C@@]45CCCN4C[C@H](F)C5)nc4c(F)c(-c5cccc6ccc(F)c(Cl)c56)ncc34)C[C@@H]2CC#N)C1 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CC(C1)O2 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1[C@@H]2CC[C@H]1COC2] 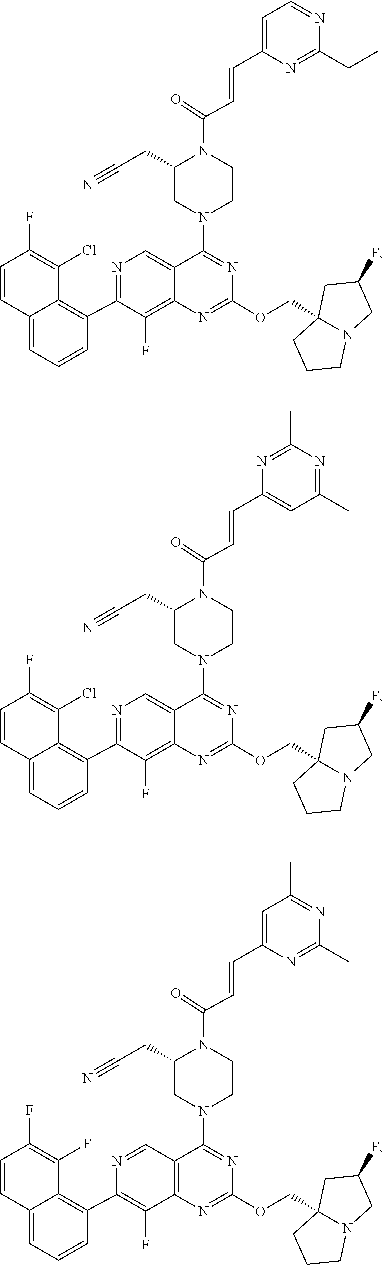 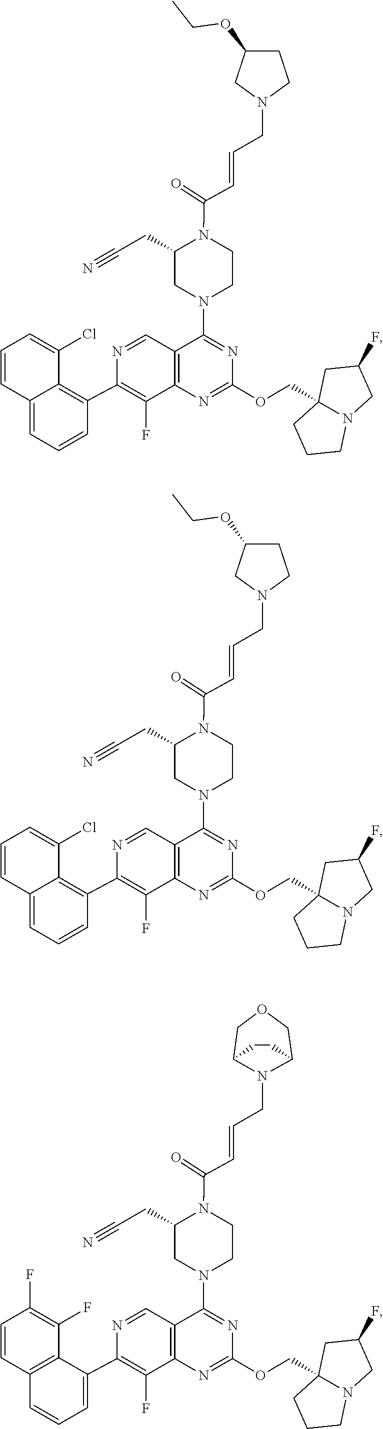 [SMILES: CCO[C@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1 ; CCO[C@@H]1CCN(C/C=C/C(=O)N2CCN(c3nc(OC[C@@]45CCCN4C[C@H](F)C5)nc4c(F)c(-c5cccc6cccc(Cl)c56)ncc34)C[C@@H]2CC#N)C1 ; N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1[C@@H]2CC[C@H]1COC2] 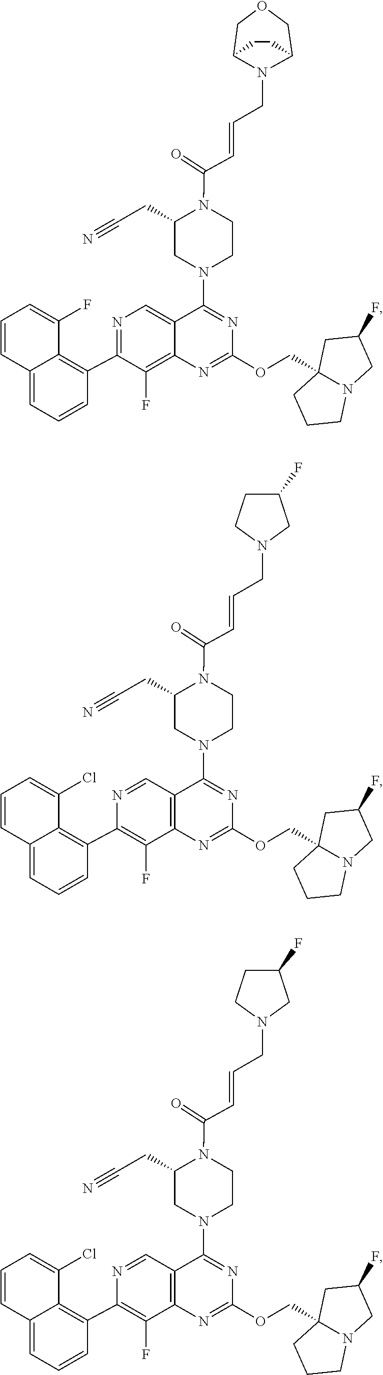 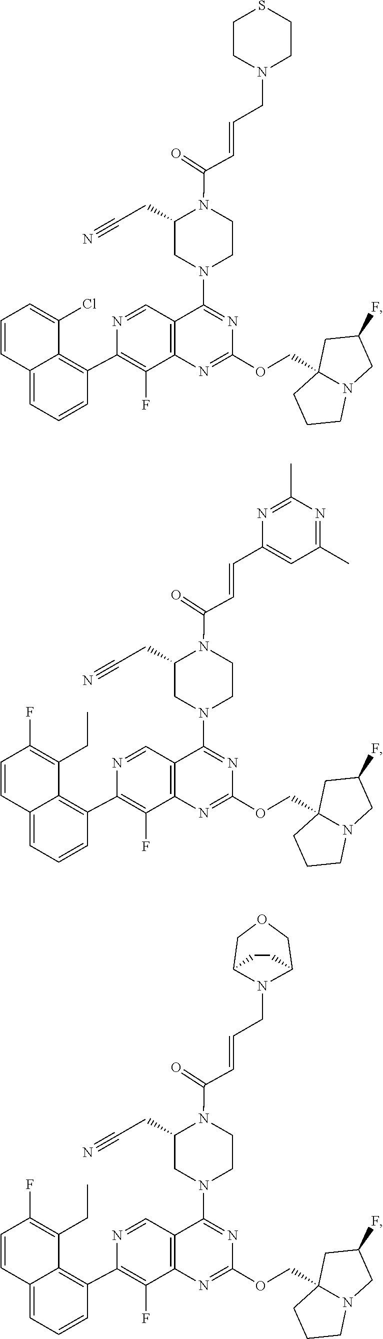 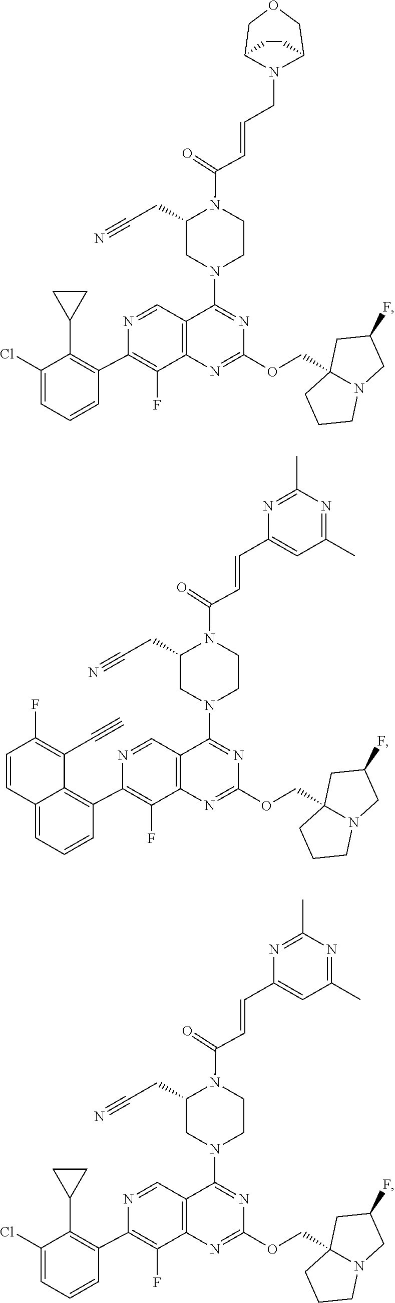 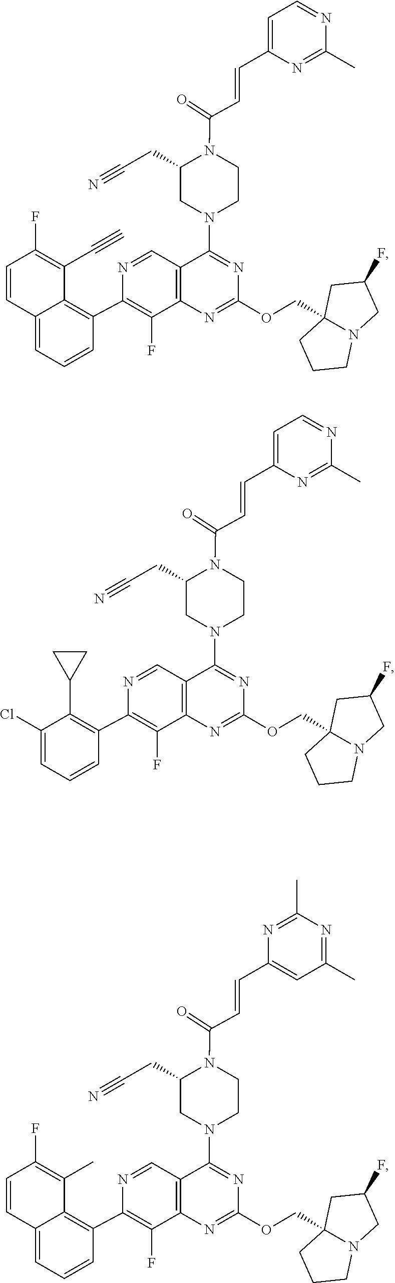 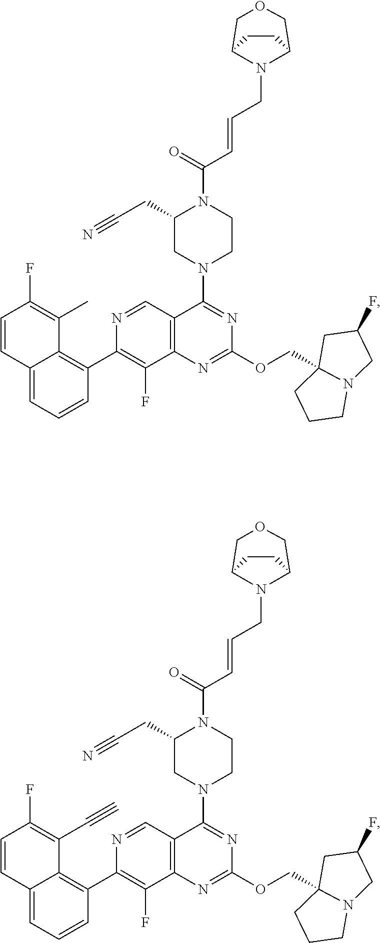 [SMILES: Cc1c(F)ccc2cccc(-c3ncc4c(N5CCN(C(=O)/C=C/CN6[C@@H]7CC[C@H]6COC7)[C@@H](CC#N)C5)nc(OC[C@@]56CCCN5C[C@H](F)C6)nc4c3F)c12 ; C#Cc1c(F)ccc2cccc(-c3ncc4c(N5CCN(C(=O)/C=C/CN6[C@@H]7CC[C@H]6COC7)[C@@H](CC#N)C5)nc(OC[C@@]56CCCN5C[C@H](F)C6)nc4c3F)c12] 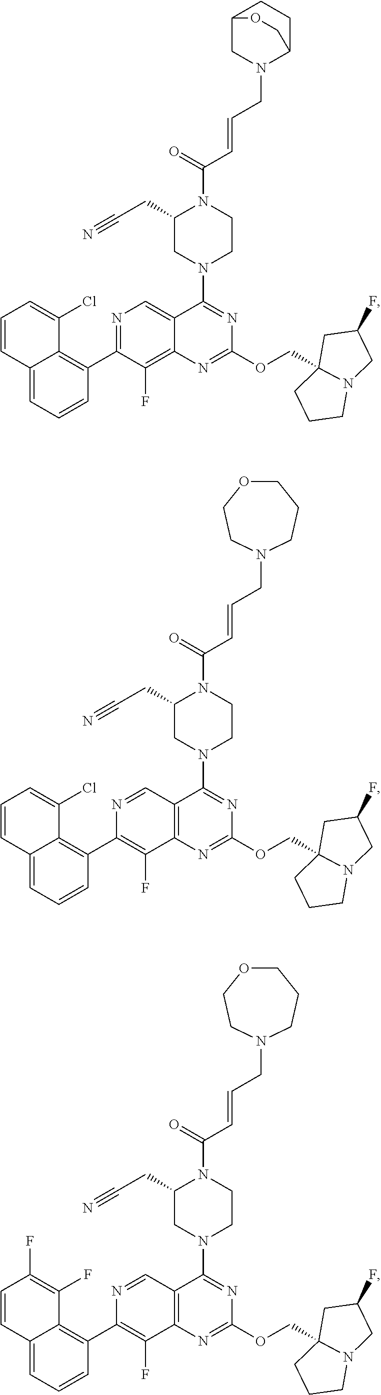 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CC2CCC1CO2 ; N#CC[C@H]1CN(c2nc(OC[C@@]34CCCN3C[C@H](F)C4)nc3c(F)c(-c4cccc5cccc(Cl)c45)ncc23)CCN1C(=O)/C=C/CN1CCCOCC1 ; N#CC[C@H]1CN(c2nc(OC[C@@]34CCCN3C[C@H](F)C4)nc3c(F)c(-c4cccc5ccc(F)c(F)c45)ncc23)CCN1C(=O)/C=C/CN1CCCOCC1] 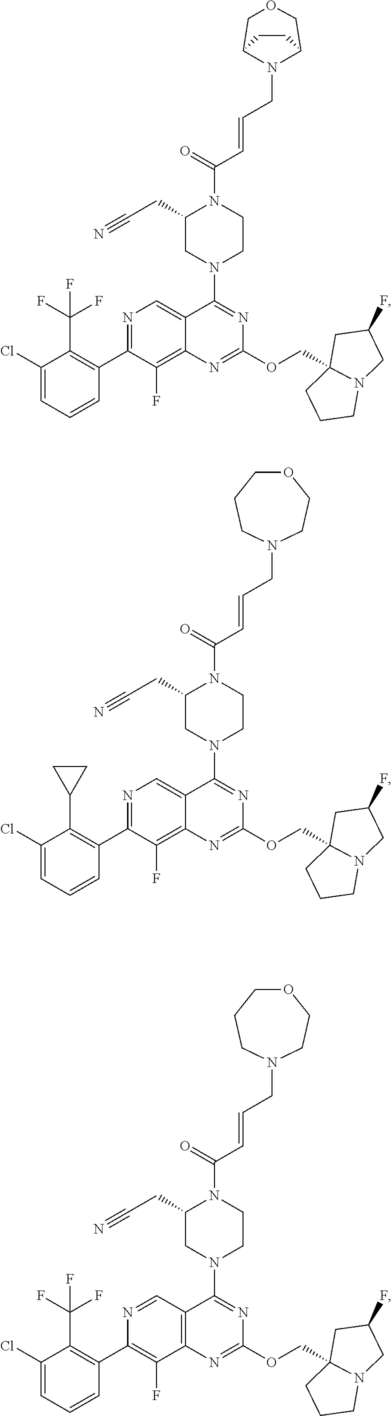 [SMILES: N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1[C@@H]2CC[C@H]1COC2 ; N#CC[C@H]1CN(c2nc(OC[C@@]34CCCN3C[C@H](F)C4)nc3c(F)c(-c4cccc(Cl)c4C4CC4)ncc23)CCN1C(=O)/C=C/CN1CCCOCC1 ; N#CC[C@H]1CN(c2nc(OC[C@@]34CCCN3C[C@H](F)C4)nc3c(F)c(-c4cccc(Cl)c4C(F)(F)F)ncc23)CCN1C(=O)/C=C/CN1CCCOCC1] 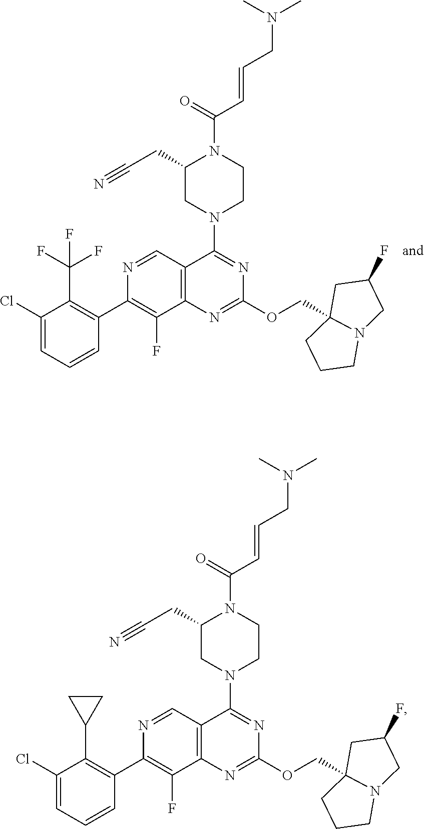 [SMILES: CN(C)C/C=C/C(=O)N1CCN(c2nc(OC[C@@]34CCCN3C[C@H](F)C4)nc3c(F)c(-c4cccc(Cl)c4C(F)(F)F)ncc23)C[C@@H]1CC#N ; CN(C)C/C=C/C(=O)N1CCN(c2nc(OC[C@@]34CCCN3C[C@H](F)C4)nc3c(F)c(-c4cccc(Cl)c4C4CC4)ncc23)C[C@@H]1CC#N]
or a salt thereof, and/or an isotopologue thereof.

116. The compound of any one of claims 1 to 115, wherein the compound is not a salt.

117. The compound of any one of claims 1 to 115, wherein the compound is a salt.

118. The compound of claim 117, wherein the salt is a pharmaceutically acceptable salt.

119. A pharmaceutical formulation comprising the compound of any one of claims 1 to 118, wherein when the compound is a salt, the salt is a pharmaceutically acceptable salt, and a pharmaceutically acceptable carrier.

120. A compound of any one of claims 1 to 118 or a pharmaceutical formulation according to claim 119, for use in treating or suppressing cancer, wherein when the compound is a salt, the salt is a pharmaceutically acceptable salt.

121. The compound or pharmaceutical composition for use of claim 120, wherein the cancer is selected from the group consisting of: lung, colorectal, pancreatic, bile duct, thyroid, gall bladder, uterine, mesothelioma, cervical, and bladder cancers.

122. The compound or pharmaceutical composition for use of claim 120, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.

123. The compound or pharmaceutical composition for use of any one of claims 120-122, wherein the cancer is a KRAS G12C mediated cancer.

124. The compound or pharmaceutical composition for use of any one of claims 120-122, wherein the subject has been diagnosed as having a KRAS G12C mediated cancer.

125. The compound or pharmaceutical composition for use of any one of claims 120-124, wherein the compound or pharmaceutical composition is configured for administration with a therapeutically effective amount of an additional chemotherapeutic agent.

126. The compound or pharmaceutical composition for use of any one of claims 120-125, wherein the compound or pharmaceutical composition is configured for administration in a therapeutically effective amount.
